(19) United States
(12) Patent Application Publication
(10) Pub. No.: US 2015/0104543 A1
(43) Pub. Date: Apr. 16, 2015

(54) ORGANIC ACID PRODUCTION BY FUNGAL CELLS

(71) Applicant: DSM IP ASSETS B.V., TE HEERLEN (NL)

(72) Inventors: Aaron Adriaan WINKLER, The Hague (NL); Abraham Frederik DE HULSTER, Pijnacker (NL); Johannes Pieter VAN DIJKEN, Leidschendam (NL); Jacobus Thomas PRONK, Schipluiden (NL); Joshua TRUEHEART, Concord, MA (US); Kevin T. MADDEN, Arlington, MA (US); Carlos Gancedo RODRIGUEZ, Majadahonda (ES); Carmen-Lisset Flores MAURIZ, Madrid (ES); Antonius Jeroen Adriaan VAN MARIS, Delft (NL); Jacob C. HARRISON, W. Newton, MA (US)

(21) Appl. No.: 14/496,057

(22) Filed: Sep. 25, 2014

Related U.S. Application Data
(63) Continuation of application No. 12/600,559, filed on Oct. 12, 2010, now abandoned.
(60) Provisional application No. 60/939,034, filed on May 18, 2007.

(51) Int. Cl. C12N 15/52; A23L 1/28; C12P 7/46

(52) U.S. Cl. CPC C12N 15/52; C12P 7/46; A23L 1/28; A23V 2002/00
USPC 426/61; 435/254.11; 435/145

(57) ABSTRACT

Improved systems for the biological production of organic acids are described.

### CROSS REFERENCE TO RELATED APPLICATIONS

This application is a continuation of U.S. Ser. No. 12/600,559, filed Oct. 12, 2010, which is a 371 National Phase application of PCT/US2008/064103, filed May 19, 2008, which claims the benefit of U.S. Provisional Patent Application Ser. No. 60/939,034, filed May 18, 2007, the contents of which are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Oct. 5, 2010, is named 23842US1.txt and is 788,707 bytes in size.

### BACKGROUND

Dicarboxylic acids are organic compounds that include two carboxylic acid groups. Such compounds find utility in a variety of commercial settings including, for example, in areas relating to food additives, polymer plasticizers, solvents, lubricants, engineered plastics, epoxy curing agents, adhesive and powder coatings, corrosion inhibitors, cosmetics, pharmaceuticals, electrolytes, etc.

Carboxylic acid groups, including those in dicarboxylic acids, are readily convertible into their ester forms. Such carboxylic acid esters are commonly employed in a variety of settings. For example, lower chain esters are often used as flavouring base materials, plasticizers, solvent carriers and/or coupling agents. Higher chain compounds are commonly used as components in metalworking fluids, surfactants, lubricants, detergents, oiling agents, emulsifiers, wetting agents textile treatments and emollients.

Carboxylic acid esters are also used as intermediates for the manufacture of a variety of target compounds. A wide range of physical properties (e.g., viscosities, specific gravities, vapor pressures, boiling points, etc.) can be achieved with different esters of the same carboxylic acid. It is therefore desirable to develop production systems for dicarboxylic acid compounds and/or their esters and/or anhydrides.

Many dicarboxylic acids, and particularly malic, fumaric, succinic, and tartaric acids, can be produced either by chemical synthesis or by fermentation. Currently, commercial scale production is typically performed by chemical synthesis or by extraction from biological sources (e.g. grape skins). Among other things, such chemical synthesis processes can generate large amounts of harmful wastes. There remains a need for the development of improved systems for producing organic acids such as dicarboxylic acids. There is a particular need for the development of biological systems for achieving such production.

### SUMMARY

The present disclosure provides improved systems for the biological production of organic acids (e.g., dicarboxylic acids). For example, the present disclosure provides systems for the biological production of an organic acid selected from the group consisting of fumaric, malic acid, succinic acid, tartaric acid, and combinations thereof.

Described herein is a recombinant fungal cell having a genetic modification that decreases pyruvate decarboxylase (PDC) activity, wherein the recombinant fungal cell, when cultured under conditions that produce a C4 dicarboxylic acid, produces more of at least one C4 dicarboxylic acid than an otherwise identical fungal cell not having the genetic modification. In some cases, the recombinant fungal cell has a genetic modification that increases malate dehydrogenase (MDH) activity.

Also described herein is a recombinant fungal cell having a genetic modification that increases malate dehydrogenase (MDH) activity, wherein the recombinant fungal cell, when cultured under conditions that produce a C4 dicarboxylic acid, produces more of at least one C4 dicarboxylic acid than an otherwise identical fungal cell not having the genetic modification.

Also described is a recombinant fungal cell having a genetic modification such that the recombinant fungal cell can be cultured to produce at least 0.5 mole of at least one C4 dicarboxylic acid/liter of culture from a feedstock containing a carbon substrate that must be assimilated through at least a portion of the glycolytic pathway.

In some cases, the recombinant fungal cell has a modification selected from the group consisting of a modification that: a) increases anaplerotic activity; b) increases or decreases organic acid transport activity; c) increases or decreases glucose sensing and regulatory polypeptide activity; d) increases or decreases hexose transporter (HXT) activity; and e) increases or decreases C4 dicarboxylic acid biosynthetic activity.

In some cases, the recombinant fungal cell has a modification selected from the group consisting of a modification that: a) increases anaplerotic activity; b) decreases PDC activity; c) increases or decreases organic acid transport activity; d) increases or decreases glucose sensing and regulatory polypeptide activity; e) increases or decreases hexose transporter (HXT) activity; and f) increases or decreases C4 dicarboxylic biosynthetic activity.

In some cases, the recombinant fungal cell has a further modification selected from the group consisting of a modification that: a) increases anaplerotic activity; b) increases or decreases organic acid transport activity; c) increases or decreases glucose sensing and regulatory polypeptide activity; d) increases or decreases hexose transporter (HXT) activity; and e) increases or decreases C4 dicarboxylic biosynthetic activity.

In some cases, the recombinant fungal cell has a modification selected from the group consisting of a modification that: a) increases anaplerotic activity; b) decreases PDC activity; c) increases or decreases organic acid transport activity; d) increases or decreases glucose sensing and regulatory polypeptide activity; e) increases or decreases hexose transporter (HXT) activity; and f) increases or decreases C4 dicarboxylic biosynthetic activity.

Also described is a recombinant fungal cell having a genetic modification that increases pyruvate carboxylase (PYC) activity or a genetic modification that increases malate dehydrogenase (MDH) activity, and at least one modification selected from the group consisting of a modification that: a) increases anaplerotic activity; b) decreases pyruvate decarboxylate (PDC) activity; c) increases or decreases organic acid transport activity; d) increases or decreases glucose sensing and regulatory polypeptide activity; e) increases or decreases hexose transporter (HXT) activity; and f) increases or decreases C4 dicarboxylic acid biosynthetic activity.

Also described is a recombinant fungal cell having a genetic modification selected from the group consisting of a modification that: a) increases anaplerotic activity; b) decreases PDC activity; c) increases or decreases organic acid transport activity; d) increases or decreases glucose sensing and regulatory polypeptide activity; e) increases or decreases hexose transporter (HXT) activity; and f) increases or decreases C4 dicarboxylic acid biosynthetic activity; and wherein said fungal cell can be cultured to produce at least 0.5 mole of C4 dicarboxylic acid per liter from a feedstock containing a carbon substrate that must be assimilated through at least a portion of the glycolytic pathway.

In some cases, the modification to increase anaplerotic activity comprises at least one modification selected from the group consisting of a modification that: a) increases pyruvate carboxylase (PYC) activity; b) increases phosphoenolpyruvate carboxylase (PPC) activity; c) increases or decreases phosphoenolpyruvate carboxykinase (PCK) activity; d) increases or decreases pyruvate kinase (PYK) activity; e) increases biotin protein ligase (BPL) activity; 0 increases biotin transport protein (VHT) activity; g) increases or decreases bicarbonate transport activity; and h) increases carbonic anhydrase activity.

In some cases, the modification to increase or decrease C4 dicarboxylic acid biosynthetic activity comprises at least one modification selected from the group consisting of a modification that: a) increases malate dehydrogenase (MDH) activity; b) increases or decreases fumarase activity; c) increases or decreases fumarate reductase activity; d) increases or decreases malate synthase activity; e) increases or decreases malic enzyme activity; 0 increases or decreases isocitrate lyase activity; g) increases or decreases ATP-citrate lyase activity; and h) increases or decreases succinate dehydrogenase activity.

In various cases: the at least one modification comprises a genetic modification that increases PYC activity; the genetic modification is the addition of a gene encoding a PYC polypeptide; the genetic modification is a genetic modification that increases the transcription or translation of a gene encoding a PYC polypeptide; the at least one genetic modification increases activity by increasing expression of the PYC polypeptide to a level above that at which it is expressed in an otherwise identical fungus that lacks the at least one genetic modification; the PYC polypeptide is active in the cytosol; the PYC polypeptide is heterologous to the fungus; the PYC polypeptide has an amino acid sequence identical to that of a PYC polypeptide from an organism of the Saccharomyces genus; the PYC polypeptide has an amino acid sequence identical to that of a Saccharomyces cerevisiae PYC polypeptide; the PYC polypeptide has at least 75% identity to SEQ ID NO:1 (PYC2); the PYC polypeptide has at least 95% identity to SEQ ID NO:1 (PYC2); the PYC polypeptide has at least 75% identity to SEQ ID NO:61 (PYC1); the PYC polypeptide has at least 95% identity to SEQ ID NO:61 (PYC1); the PYC polypeptide has the amino sequence of a PYC2-ext polypeptide; the PYC polypeptide has at least 75% identity to SEQ ID NO:65 (PYC2-ext); the PYC polypeptide has at least 95% identity to SEQ ID NO:65 (PYC2-ext); the PYC polypeptide has the amino acid sequence of a Y. lipolytica PYC1 polypeptide; the PYC polypeptide has at least 75% identity to SEQ ID NO:67 (Y. lipolytica PYC1); the PYC polypeptide has at least 95% identity to SEQ ID NO:67 (Y. lipolytica PYC1); the PYC polypeptide has the amino acid sequence of an A. niger pycA polypeptide; the PYC polypeptide has at least 75% identity to SEQ ID NO:69 (A. niger pycA); the PYC polypeptide has at least 95% identity to SEQ ID NO:69 (A. niger pycA); the PYC polypeptide has an amino acid sequence identical to that of a Nocardia sp. JS614 pycA polypeptide; the PYC polypeptide has at least 75% identity to SEQ ID NO:71 (Nocardia sp. JS614 pycA); the PYC polypeptide has at least 95% identity to SEQ ID NO:71 (Nocardia sp. JS614 pycA); the PYC polypeptide has the amino acid sequence of a Methanothermobacter thermautotrophicus str. Delta H pycA polypeptide; the PYC polypeptide has at least 75% identity to SEQ ID NO:73 (Methanothermobacter thermautotrophicus str. Delta H pycA); the PYC polypeptide has at least 95% identity to SEQ ID NO:73 (Methanothermobacter thermautotrophicus str. Delta H pycA); the PYC polypeptide has the amino acid sequence of a Methanothermobacter thermautotrophicus str. Delta H pycB polypeptide; the PYC polypeptide has at least 75% identity to SEQ ID NO:75 (Methanothermobacter thermautotrophicus str. Delta H pycB); the PYC polypeptide has at least 95% identity to SEQ ID NO:75 (Methanothermobacter thermautotrophicus str. Delta H pycB); the PYC polypeptide has the amino acid sequence of a PYC polypeptide in FIG. 33; the PYC polypeptide has at least 75% identity to a PYC polypeptide in FIG. 33; the PYC polypeptide has at least 95% identity to a PYC polypeptide in FIG. 33.

In some cases, the at least one modification comprises a genetic modification that increases the activity of a phosphoenol pyruvate carboxylase (PPC) polypeptide as compared with its activity in an otherwise identical fungus lacking the modification; the genetic modification increases activity of the PPC by increasing its expression; the genetic modification is the addition of a gene encoding a PPC polypeptide; the genetic modification is the genetic modification that increases the transcription of a gene encoding a PPC polypeptide or increases translation of a gene encoding a PPC polypeptide; the fungus contains a modification to decrease sensitivity of the PPC polypeptide to inhibition by one more of malate, aspartate, and oxaloacetate; the PPC polypeptide is heterologous to the fungus; the PPC polypeptide has an amino acid sequence identical to that of a PPC polypeptide from an organism of the Escherichia genus; the PPC polypeptide has the amino acid sequence of an Escherichia coli PPC polypeptide the PPC polypeptide has at least 75% identity to SEQ ID NO:7 (E. coli PPC); the PPC polypeptide has at least 95% identity to SEQ ID NO:7 (E. coli PPC); the PPC polypeptide has an amino acid sequence identical to that of an Escherichia coli mut5-K620S Ppc polypeptide; the PPC polypeptide has at least 75% identity to SEQ ID NO:51 (Escherichia coli mut5-K620S Ppc); PPC polypeptide has at least 95% identity to SEQ ID NO:51 (Escherichia coli mut5-K620S Ppc); the PPC polypeptide has an amino acid sequence identical to that of an Escherichia coli mut10-K773G Ppc polypeptide; the PPC polypeptide has at least 75% identity to SEQ ID NO:53 (Escherichia coli mut10-K773G Ppc); the PPC polypeptide has at least 95% identity to SEQ ID NO:53 (Escherichia coli mut10-K773G Ppc); the PPC polypeptide has an amino acid sequence identical to that of an Erwinia carotovora Ppc polypeptide; the PPC polypeptide has at least 75% identity to SEQ ID NO:55 (Erwinia carotovora Ppc); the PPC polypeptide has at least 95% identity to SEQ ID NO:55 (Erwinia carotovora Ppc); the PPC polypeptide has an amino acid sequence identical to that of a (Thermo)synechococcus vulcanus Ppc polypeptide; the PPC polypeptide has at least 75% identity to SEQ ID NO:57 ((Thermo)synechococcus vulcanus Ppc); the PPC polypeptide has at least 95% identity to SEQ ID NO:57 ((Thermo)synechococcus vulcanus Ppc); the PPC polypeptide has an amino acid sequence identical to that of a Corynebacterium glutamicum Ppc polypeptide; the PPC polypeptide has at least 75% identity to SEQ ID NO:59 (Corynebacterium glutamicum Ppc); the PPC polypeptide has at least 95% identity to SEQ ID NO:59 (Corynebacterium glutamicum Ppc); the PPC polypeptide has the amino acid sequence of a PPC polypeptide in FIG. 32; the PPC polypeptide has at least 75% identity to a PPC polypeptide in FIG. 32; the PPC polypeptide has at least 95% identity to a PPC polypeptide in FIG. 32; the PPC polypeptide has at least 98% identity to a PPC polypeptide FIG. 32.

In some cases, the at least one modification comprises a genetic modification that increases or decreases the activity of a phosphoenol pyruvate carboxykinase (PCK) polypeptide as compared with its activity in an otherwise identical fungus lacking the modification; the genetic modification increases or decreases activity of the PCK polypeptide by increasing or decreasing its expression to a level above or below that at which it is expressed in an otherwise identical fungus that lacks the at least one genetic modification; the genetic modification that increases expression is the addition of a gene encoding a PCK polypeptide; the genetic modification that increases expression is a genetic modification that increases the transcription or translation of a gene encoding a PCK polypeptide; the genetic modification that decreases expression is the deletion of all or part of a gene encoding a PCK polypeptide or the disruption of a gene encoding a PCK polypeptide; the PCK polypeptide is heterologous to the fungus; the PCK polypeptide has the amino acid sequence of an Erwinia carotovora Pck polypeptide; the PCK polypeptide has at least 75% identity to SEQ ID NO:157 (Erwinia carotovora pckA); the PCK polypeptide has at least 95% identity to SEQ ID NO:157 (Erwinia carotovora pckA); the PCK polypeptide has the amino acid sequence of an Actinobacillus pleuropneumoniae Pck polypeptide; the PCK polypeptide has at least 75% identity to SEQ ID NO:159 (Actinobacillus pleuropneumoniae pckA); the PCK polypeptide has at least 95% identity to SEQ ID NO:159 (Actinobacillus pleuropneumoniae pckA); the PCK polypeptide has the amino acid sequence of an Actinobacillus succinogenes Pck polypeptide; the PCK polypeptide has at least 75% identity to SEQ ID NO:161 (Actinobacillus succinogenes pckA); the PCK polypeptide has at least 95% identity to SEQ ID NO:161 (Actinobacillus succinogenes pckA); the PCK polypeptide has the amino acid sequence of a Saccharomyces cerevisiae Pck polypeptide; the PCK polypeptide has at least 75% identity to SEQ ID NO:163 (Saccharomyces cerevisiae Pck1); the PCK polypeptide has at least 95% identity to SEQ ID NO:163 (Saccharomyces cerevisiae Pck1); the PCK polypeptide has an amino acid sequence identical to a PCK polypeptide in FIG. 36; the PCK polypeptide has at least 75% identity to a PCK polypeptide in FIG. 36; the PCK polypeptide has at least 95% identity to a PCK polypeptide in FIG. 36; the at least one modification comprises a genetic modification that increases BPL (biotin protein ligase) activity; the at least one genetic modification increases activity by increasing expression of a BPL polypeptide to a level above that at which it is expressed in an otherwise identical fungus that lacks the at least one genetic modification; the genetic modification that increases expression is the addition of a gene encoding a BPL polypeptide; the genetic modification that increases expression is a genetic modification that increases the transcription or translation of a gene encoding a BPL polypeptide; the BPL polypeptide is heterologous to the fungus; the BPL polypeptide has an amino acid sequence identical to that of a BPL polypeptide from an organism of the Saccharomyces genus the BPL polypeptide has an amino acid sequence identical to that of a Saccharomyces cerevisiae BPL polypeptide; the BPL polypeptide has at least 75% identity to SEQ ID NO:95 (S. cerevisiae BPL1); the BPL polypeptide has at least 95% identity to SEQ ID NO:95 (S. cerevisiae BPL1); the BPL polypeptide has an amino acid sequence identical to a BPL polypeptide in FIG. 46; the BPL polypeptide has at least 75% identity to a BPL polypeptide in FIG. 46; the BPL polypeptide has at least 95% identity to a BPL polypeptide in FIG. 46.

In some cases, the at least one modification comprises a genetic modification that increases VHT activity; the at least one genetic modification increases activity by increasing expression of a VHT polypeptide to a level above that at which it is expressed in an otherwise identical fungus that lacks the at least one genetic modification; the genetic modification that increases expression is the addition of a gene encoding a VHT polypeptide; the genetic modification that increases expression is a genetic modification that increases the transcription or translation of a gene encoding a VHT polypeptide; the VHT polypeptide is heterologous to the fungus; the VHT polypeptide has an amino acid sequence identical to that of a VHT polypeptide from an organism of the Saccharomyces genus; the VHT polypeptide has an amino acid sequence identical to that of a Saccharomyces cerevisiae VHT polypeptide; the VHT polypeptide has at least 75% identity to SEQ ID NO:97 (S. cerevisiae VHT1); the VHT polypeptide has at least 95% identity to SEQ ID NO:97 (S. cerevisiae VHT1); the VHT polypeptide has an amino acid sequence identical to a VHT polypeptide in FIG. 48; the VHT polypeptide has at least 75% identity to a VHT polypeptide in FIG. 48; wherein the VHT polypeptide has at least 95% identity to a VHT polypeptide in FIG. 48; the at least one modification comprises a genetic modification that increases or decreases bicarbonate transport activity.

In some cases, the at least one genetic modification increases or decreases bicarbonate transport activity by increasing or decreasing expression of a bicarbonate transport polypeptide to a level above or below that at which it is expressed in an otherwise identical fungus that lacks the at least one genetic modification; the genetic modification that increases expression is the addition of a gene encoding a bicarbonate transport polypeptide; the genetic modification that increases expression is a genetic modification that increases the transcription or translation of a gene encoding a bicarbonate transport polypeptide; the genetic modification that decreases expression is the deletion of all or part of a gene encoding a bicarbonate transport polypeptide or the disruption of a gene encoding a bicarbonate transport polypeptide; the bicarbonate transport polypeptide is heterologous to the fungus; the bicarbonate transport polypeptide has an amino acid sequence identical to that of a bicarbonate transport polypeptide from an organism of the Saccharomyces genus; the bicarbonate transport polypeptide has an amino acid sequence identical to that of a Saccharomyces cerevisiae bicarbonate transport polypeptide; the bicarbonate transport polypeptide has at least 75% identity to SEQ ID NO:89 (S. cerevisiae YNL275w); the bicarbonate transport polypeptide has at least 95% identity to SEQ ID NO:89 (S. cerevisiae YNL275w); the bicarbonate transport polypeptide has an amino acid sequence identical to SEQ ID NO:91 (H. sapiens SLC4A1); the bicarbonate transport polypeptide has at least 75% identity to SEQ ID NO:91 (H. sapiens SLC4A1); the bicarbonate transport polypeptide has at least 95% identity to SEQ ID NO:91 (H. sapiens SLC4A1); the bicarbonate transport polypeptide has an amino acid sequence identical to SEQ ID NO:93 (Oryctolagus cuniculus SLC4A9): the bicarbonate transport polypeptide has at least 75% identity to SEQ ID NO:93 (Oryctolagus cuniculus SLC4A9); the bicarbonate transport polypeptide has at least 95% identity to SEQ ID NO:93 (Oryctolagus cuniculus SLC4A9); the bicarbonate transport polypeptide has an amino acid sequence identical to a bicarbonate transport polypeptide in FIG. 39; the bicarbonate transport polypeptide has at least 75% identity to a bicarbonate transport polypeptide in FIG. 39; the bicarbonate transport polypeptide has at least 95% identity to a bicarbonate transport polypeptide in FIG. 39.

In some cases, the at least one modification comprises a genetic modification that increases carbonic anhydrase activity; the at least one genetic modification increases carbonic anhydrase activity by increasing expression of the carbonic anhydrase polypeptide to a level above that at which it is expressed in an otherwise identical fungus that lacks the at least one genetic modification; the genetic modification that increases expression is the addition of a gene encoding a carbonic anhydrase polypeptide; the genetic modification that increases expression is a genetic modification that increases the transcription or translation of a gene encoding a carbonic anhydrase polypeptide; the carbonic anhydrase polypeptide is heterologous to the fungus; the carbonic anhydrase polypeptide has an amino acid sequence identical to that of a carbonic anhydrase polypeptide from an organism of the Saccharomyces genus; the carbonic anhydrase polypeptide has an amino acid sequence identical to that of a Saccharomyces cerevisiae carbonic anhydrase polypeptide; the carbonic anhydrase polypeptide has at least 75% identity to SEQ ID NO:99 (S. cerevisiae NCE103); the carbonic anhydrase polypeptide has at least 95% identity to SEQ ID NO:99 (S. cerevisiae NCE103); the carbonic anhydrase polypeptide has an amino acid sequence identical to a carbonic anhydrase polypeptide in FIG. 40; the carbonic anhydrase polypeptide has at least 75% identity to a carbonic anhydrase polypeptide in FIG. 40; the carbonic anhydrase polypeptide has at least 95% identity to a carbonic anhydrase polypeptide in FIG. 40.

In some cases, the at least one modification comprises a genetic modification that increases MDH activity; the genetic modification increases activity by increasing expression of the MDH; the genetic modification that increases expression is the addition of a gene encoding a MDH polypeptide; the genetic modification that increases expression is a genetic modification that increases the transcription or translation of a gene encoding a MDH polypeptide; the MDH polypeptide is active in the cytosol; the MDH polypeptide is targeted to the cytosol of the fungal cell by modification of its coding region; the fungal cell contains a modification that decreases sensitivity of the MDH polypeptide to inhibition in the presence of glucose; the fungal cell has at least 2-fold the MDH polypeptide activity in the presence of glucose, when compared to an otherwise identical parental strain lacking the modification that decreases sensitivity of the MDH polypeptide to inhibition in the presence of glucose; the MDH polypeptide is heterologous to the fungal cell; the MDH polypeptide has an amino acid sequence identical to that of an MDH polypeptide from an organism of the Saccharomyces genus; the MDH polypeptide has an amino acid sequence identical to that of a Saccharomyces cerevisiae MDH polypeptide; the MDH polypeptide is selected from the group consisting of: MDH1, MDH2, or MDH3 and combinations thereof; the MDH polypeptide has an amino acid sequence identical to that of an S. cerevisiae MDH1 polypeptide; the MDH1 polypeptide has at least 75% identity to SEQ ID NO:9 (S.c. MDH1); the MDH1 polypeptide has at least 95% identity to SEQ ID NO:9 (S.c. MDH1); the MDH polypeptide has an amino acid sequence identical to that of an S. cerevisiae MDH2 polypeptide; the MDH2 polypeptide has at least 75% identity to SEQ ID NO:11 (S.c. MDH2); the MDH2 polypeptide has at least 95% identity to SEQ ID NO:11 (S.c. MDH2); the MDH polypeptide has an amino acid sequence identical to that of an S. cerevisiae MDH3 polypeptide; the MDH3 polypeptide has at least 75% identity to SEQ ID NO:15 (S.c. MDH3); the MDH3 polypeptide has at least 95% identity to SEQ ID NO:15 (S.c. MDH3); the MDH polypeptide has an amino acid sequence identical to that of a MDH2 P2S polypeptide; the MDH polypeptide has at least 75% identity to SEQ ID NO:13 (MDH2 P2S); the MDH polypeptide has at least 95% identity to SEQ ID NO:13 (MDH2 P2S); the MDH polypeptide has an amino acid sequence identical to that of an Actinobacillus succinogenes MDH polypeptide; the MDH polypeptide has at least 75% identity to SEQ ID NO:19 (Actinobacillus succinogenes MDH); the MDH polypeptide has at least 95% identity to SEQ ID NO:19 (Actinobacillus succinogenes MDH); the MDH polypeptide has an amino acid sequence identical to that of a Yarrowia lipolytica MDH polypeptide; the MDH polypeptide has at least 75% identity to SEQ ID NO:21 (Yarrowia lipolytica MDH); the MDH polypeptide has at least 95% identity to SEQ ID NO:21 (Yarrowia lipolytica MDH); the MDH polypeptide has an amino acid sequence identical to that of an Aspergillus niger MDH polypeptide; the MDH polypeptide has at least 75% identity to SEQ ID NO:23 (Aspergillus niger MDH); the MDH polypeptide has at least 95% identity to SEQ ID NO:23 (Aspergillus niger MDH); the MDH polypeptide has an amino acid sequence identical to that of an MDH polypeptide in FIG. 34; the MDH polypeptide has at least 75% identity to a MDH polypeptide in FIG. 34; the MDH polypeptide has at least 95% identity to a MDH polypeptide in FIG. 34; the MDH polypeptide is MDH3ΔSKL.

In some cases, the at least one modification comprises a genetic modification that increases or decreases fumarase activity; the at least one genetic modification increases activity by increasing or decreasing expression of a fumarase polypeptide to a level above or below that at which it is expressed in an otherwise identical fungus that lacks the at least one genetic modification; the genetic modification that increases expression is the addition of a gene encoding a fumarase polypeptide; the genetic modification that increases expression is a genetic modification that increases the transcription or translation of a gene encoding a fumarase polypeptide; the genetic modification that decreases expression is the deletion of all or part of a gene encoding a fumarase polypeptide or the disruption of a gene encoding a fumarase polypeptide; the fumarase polypeptide is heterologous to the fungus; the fumarase polypeptide has an amino acid sequence identical to that of a fumarase polypeptide from an organism of the Saccharomyces genus; the fumarase polypeptide has an amino acid sequence identical to that of a Saccharomyces cerevisiae fumarase polypeptide; the fumarase polypeptide has at least 75% identity to SEQ ID NO:101 (S. cerevisiae FUM1); the fumarase polypeptide has at least 95% identity to SEQ ID NO:101 (S. cerevisiae FUM1); the fumarase polypeptide has an amino acid sequence identical to a fumarase polypeptide in FIG. 43; the fumarase polypeptide has at least 75% identity to a fumarase polypeptide in FIG. 43; the fumarase polypeptide has at least 95% identity to a fumarase polypeptide in FIG. 43.

In some cases, the at least one modification comprises a genetic modification that increases or decreases fumarate reductase activity; the at least one genetic modification increases activity by increasing or decreasing expression of the fumarate reductase polypeptide to a level above or below that at which it is expressed in an otherwise identical fungus that lacks the at least one genetic modification; the genetic modification that increases expression is the addition of a gene encoding a fumarate reductase polypeptide; the genetic modification that increases expression is a genetic modification that increases the transcription or translation of a gene encoding a fumarate reductase polypeptide; the genetic modification that decreases expression is the deletion of all or part of a gene encoding a fumarase reductase polypeptide or the disruption of a gene encoding a fumarate reductase polypeptide; the fumarate reductase polypeptide is heterologous to the fungus; the fumarate reductase polypeptide has an amino acid sequence identical to that of a fumarate reductase polypeptide from an organism of the Saccharomyces genus; the fumarate reductase polypeptide has an amino acid sequence identical to that of a Saccharomyces cerevisiae fumarate reductase polypeptide; the fumarate reductase polypeptide has at least 75% identity to SEQ ID NO:103 (S. cerevisiae OSM1); the fumarate reductase polypeptide has at least 95% identity to SEQ ID NO:103 (S. cerevisiae OSM1); the fumarate reductase polypeptide has at least 75% identity to SEQ ID NO:105 (S. cerevisiae FRDS1); the fumarate reductase polypeptide has at least 95% identity to SEQ ID NO:105 (S. cerevisiae FRDS1); the fumarate reductase polypeptide has an amino acid sequence identical to a fumarate reductase polypeptide in FIG. 42; the fumarate reductase polypeptide has at least 75% identity to a fumarate reductase polypeptide in FIG. 42; the fumarate reductase polypeptide has at least 95% identity to a fumarate reductase polypeptide in FIG. 42.

In some cases, the at least one modification comprises a genetic modification that increases or decreases malate synthase activity; the at least one genetic modification increases activity by increasing or decreasing expression of a malate synthase polypeptide to a level above or below that at which it is expressed in an otherwise identical fungus that lacks the at least one genetic modification the genetic modification that increases expression is the addition of a gene encoding a malate synthase polypeptide; the genetic modification that increases expression is a genetic modification that increases the transcription or translation of a gene encoding a malate synthase polypeptide; the genetic modification that decreases expression is the deletion of all or part of a gene encoding a malate synthase polypeptide or the disruption of a gene encoding a malate synthase polypeptide; the malate synthase polypeptide is heterologous to the fungus; the malate synthase polypeptide has an amino acid sequence identical to that of a malate synthase polypeptide from an organism of the Saccharomyces genus; the malate synthase polypeptide has an amino acid sequence identical to that of a Saccharomyces cerevisiae malate synthase polypeptide; the malate synthase polypeptide has at least 75% identity to SEQ ID NO:151 (S. cerevisiae MLS 1); the malate synthase polypeptide has at least 95% identity to SEQ ID NO:151 (S. cerevisiae MLS 1); the malate synthase polypeptide has an amino acid sequence identical to that of a Saccharomyces cerevisiae DAL7 polypeptide; the malate synthase polypeptide has at least 75% identity to SEQ ID NO:153 (S. cerevisiae DAL7); the malate synthase polypeptide has at least 95% identity to SEQ ID NO:153 (S. cerevisiae DAL7); the malate synthase polypeptide has an amino acid sequence identical to a malate synthase polypeptide in FIG. 37; the malate synthase polypeptide has at least 75% identity to a malate synthase polypeptide in FIG. 37; the malate synthase polypeptide has at least 95% identity to a malate synthase polypeptide in FIG. 37.

In some cases, the at least one modification comprises a genetic modification that increases or decreases malic enzyme activity; the at least one genetic modification increases activity by increasing or decreasing expression of a malic enzyme polypeptide to a level above or below that at which it is expressed in an otherwise identical fungus that lacks the at least one genetic modification; the genetic modification that increases expression is the addition of a gene encoding a malic enzyme polypeptide; the genetic modification that increases expression is a genetic modification that increases the transcription or translation of a gene encoding a malic enzyme polypeptide; the genetic modification that decreases expression is the deletion of all or part of a gene encoding a malic enzyme polypeptide or the disruption of a gene encoding a malic enzyme polypeptide; the malic enzyme polypeptide is heterologous to the fungus; the malic enzyme polypeptide has an amino acid sequence identical to that of a malic enzyme polypeptide from an organism of the Saccharomyces genus; the malic enzyme polypeptide has an amino acid sequence identical to that of a Saccharomyces cerevisiae malic enzyme polypeptide; the malic enzyme polypeptide has at least 75% identity to SEQ ID NO:155 (S. cerevisiae MAE1); the malic enzyme polypeptide has at least 95% identity to SEQ ID NO:155 (S. cerevisiae MAE1); the malic enzyme polypeptide has an amino acid sequence identical to a malic enzyme polypeptide in FIG. 38; the malic enzyme polypeptide has at least 75% identity to a malic enzyme polypeptide in FIG. 38; the malic enzyme polypeptide has at least 95% identity to a malic enzyme polypeptide in FIG. 38.

In some cases, the at least one modification comprises a genetic modification that increases or decreases isocitrate lyase activity; the at least one genetic modification increases activity by increasing or decreasing expression of a isocitrate lyase polypeptide to a level above or below that at which it is expressed in an otherwise identical fungus that lacks the at least one genetic modification; the genetic modification that increases expression is the addition of a gene encoding an isocitrate lyase polypeptide; the genetic modification that increases expression is a genetic modification that increases the transcription or translation of a gene encoding an isocitrate lyase polypeptide; the genetic modification that decreases expression is the deletion of all or part of a gene encoding an isocitrate lyase polypeptide or the disruption of a gene encoding an isocitrate lyase polypeptide; the isocitrate lyase polypeptide is heterologous to the fungus; the isocitrate lyase polypeptide has an amino acid sequence identical to that of an isocitrate lyase polypeptide from an organism of the Saccharomyces genus; the isocitrate lyase polypeptide has an amino acid sequence identical to that of a Saccharomyces cerevisiae isocitrate lyase polypeptide; the isocitrate lyase polypeptide has at least 75% identity to SEQ ID NO:149 (S. cerevisiae ICL1); the isocitrate lyase polypeptide has at least 95% identity to SEQ ID NO:149 (S. cerevisiae ICL1); the isocitrate lyase polypeptide has an amino acid sequence identical to an isocitrate lyase polypeptide in FIG. 49; the isocitrate lyase polypeptide has at least 75% identity to an isocitrate lyase polypeptide in FIG. 49; the isocitrate lyase polypeptide has at least 95% identity to an isocitrate lyase polypeptide in FIG. 49.

In some cases, the at least one modification comprises a genetic modification that increases or decreases ATP-citrate lyase activity; the at least one genetic modification increases activity by increasing or decreasing expression of an ATP-citrate lyase polypeptide to a level above or below that at which it is expressed in an otherwise identical fungus that lacks the at least one genetic modification; the genetic modification that increases expression is the addition of a gene encoding an ATP-citrate lyase polypeptide; the genetic modification that increases expression is a genetic modification that increases the transcription or translation of a gene encoding an ATP-citrate lyase polypeptide; the genetic modification that decreases expression is the deletion of all or part of a gene encoding an ATP-citrate lyase polypeptide or the disruption of a gene encoding an ATP-citrate lyase polypeptide; the ATP-citrate lyase polypeptide is heterologous to the fungus; the ATP-citrate lyase polypeptide has an amino acid sequence identical to that of an ATP-citrate lyase polypeptide from an organism of the Saccharomyces genus; the ATP-citrate lyase polypeptide has an amino acid sequence identical to SEQ ID NO:85 (Y. lipolytica subunit 1 (XP_{—}504787)); the ATP-citrate lyase polypeptide has at least 75% identity to SEQ ID NO:85 (Y. lipolytica subunit 1 (XP_{—}504787)); the ATP-citrate lyase polypeptide has at least 95% identity to SEQ ID NO:85 (Y. lipolytica subunit 1 (XP_{—}504787)); the ATP-citrate lyase polypeptide has an amino acid sequence identical to SEQ ID NO:85 (Y. lipolytica subunit 1 (XP_{—}503231)): the ATP-citrate lyase polypeptide has at least 75% identity to SEQ ID NO:87 (Y. lipolytica subunit 2 (XP_{—}503231)); the ATP-citrate lyase polypeptide has at least 95% identity to SEQ ID NO:87 (Y. lipolytica subunit 2 (XP_{—}503231)); the ATP-citrate lyase polypeptide has an amino acid sequence identical to an ATP-citrate lyase polypeptide in FIG. 41a or FIG. 41b1 the ATP-citrate lyase polypeptide has at least 75% identity to an ATP-citrate lyase polypeptide in FIG. 41a or FIG. 41b; the ATP-citrate lyase polypeptide has at least 95% identity to an ATP-citrate lyase polypeptide in FIG. 41a or FIG. 41b.

In some cases, the at least one modification comprises a genetic modification that increases or decreases succinate dehydrogenase activity; the at least one genetic modification increases activity by increasing or decreasing expression of a succinate dehydrogenase polypeptide to a level above or below that at which it is expressed in an otherwise identical fungus that lacks the at least one genetic modification; the genetic modification that increases expression is the addition of a gene encoding a succinate dehydrogenase polypeptide; the genetic modification that increases expression is a genetic modification that increases the transcription or translation of a gene encoding a succinate dehydrogenase polypeptide; the genetic modification that decreases expression is the deletion of all or part of a gene encoding a succinate dehydrogenase polypeptide or the disruption of a gene encoding a succinate dehydrogenase polypeptide; the succinate dehydrogenase polypeptide is heterologous to the fungus; the succinate dehydrogenase polypeptide has an amino acid sequence identical to that of a succinate dehydrogenase polypeptide from an organism of the Saccharomyces genus; the succinate dehydrogenase polypeptide has at least 75% identity to SEQ ID NO:169 (S. cerevisiae SDH1); the succinate dehydrogenase polypeptide has at least 95% identity to SEQ ID NO:169 (S. cerevisiae SDH1); the succinate dehydrogenase polypeptide has at least 75% identity to SEQ ID NO:171 (S. cerevisiae SDH2); the succinate dehydrogenase polypeptide has at least 95% identity to SEQ ID NO:171 (S. cerevisiae SDH2); the succinate dehydrogenase polypeptide has at least 75% identity to SEQ ID NO:173 (S. cerevisiae SDH3); the succinate dehydrogenase polypeptide has at least 95% identity to SEQ ID NO:173 (S. cerevisiae SDH3); the succinate dehydrogenase polypeptide has at least 75% identity to SEQ ID NO:175 (S. cerevisiae SDH4); the succinate dehydrogenase polypeptide has at least 95% identity to SEQ ID NO:175 (S. cerevisiae SDH4); the succinate dehydrogenase polypeptide has an amino acid sequence identical to a succinate dehydrogenase polypeptide in FIG. 47; the succinate dehydrogenase polypeptide has at least 75% identity to a succinate dehydrogenase polypeptide in FIG. 47; the succinate dehydrogenase polypeptide has at least 95% identity to a succinate dehydrogenase polypeptide in FIG. 47.

In some cases, the at least one modification comprises a genetic modification that increases or decreases pyruvate kinase activity; the at least one genetic modification increases activity by increasing or decreasing expression of a pyruvate kinase polypeptide to a level above or below that at which it is expressed in an otherwise identical fungus that lacks the at least one genetic modification; the genetic modification that increases expression is the addition of a gene encoding a pyruvate kinase polypeptide; the genetic modification that increases expression is a genetic modification that increases the transcription or translation of a gene encoding a pyruvate kinase polypeptide; the genetic modification that decreases expression is the deletion of all or part of a gene encoding a pyruvate kinase polypeptide or the disruption of a gene encoding a pyruvate kinase polypeptide; the pyruvate kinase polypeptide is heterologous to the fungus; the pyruvate kinase polypeptide has an amino acid sequence identical to that of a pyruvate kinase polypeptide from an organism of the Saccharomyces genus; the pyruvate kinase polypeptide has at least 75% identity to SEQ ID NO:165 (S. cerevisiae PYK1); the pyruvate kinase polypeptide has at least 95% identity to SEQ ID NO:165 (S. cerevisiae PYK1); the pyruvate kinase polypeptide has at least 75% identity to SEQ ID NO:167 (S. cerevisiae PYK2); the pyruvate kinase polypeptide has at least 95% identity to SEQ ID NO:167 (S. cerevisiae PYK2); the pyruvate kinase polypeptide has an amino acid sequence identical to a pyruvate kinase polypeptide in FIG. 45; the pyruvate kinase polypeptide has at least 75% identity to a pyruvate kinase polypeptide in FIG. 45; the pyruvate kinase polypeptide has at least 95% identity to a pyruvate kinase polypeptide in FIG. 45.

In some cases, the modification to decrease pyruvate decarboxylase (PDC) activity comprises at least one modification selected from the group consisting of a modification to decrease PDC1, PDC2, PDC5, or PDC6 activity; the modification to decrease PDC polypeptide activity comprises modifications to decrease each of PDC1, PDC5, and PDC6 activities; the modification to decrease PDC activity comprises modifications to decrease each of PDC1 and PDC5 activities; the genetic modification decreases activity by decreasing expression of a PDC polypeptide; the genetic modification that decreases expression is the deletion of all or part of a gene encoding a PDC polypeptide or the disruption of a gene encoding a PDC polypeptide; the PDC polypeptide is heterologous to the fungal cell; the PDC polypeptide has an amino acid sequence identical to that of a PDC polypeptide from an organism of the Saccharomyces genus; the PDC polypeptide has an amino acid sequence identical to that of a Saccharomyces cerevisiae PDC polypeptide; the PDC polypeptide is selected from the group consisting of: PDC1, PDC2, PDC5 or PDC6, and combinations thereof; the PDC polypeptide has an amino acid sequence identical to that of a S. cerevisiae PDC1 polypeptide; the PDC polypeptide has at least 75% identity to SEQ ID NO:77 (S.c. PDC1); the PDC polypeptide has at least 95% identity to SEQ ID NO:77 (S.c. PDC1); the PDC polypeptide has an amino acid sequence identical to that of a S. cerevisiae PDC2 polypeptide; the PDC polypeptide has at least 75% identity to SEQ ID NO:83 (S.c. PDC2); the PDC polypeptide has at least 95% identity to SEQ ID NO:83 (S.c. PDC2); the PDC polypeptide has an amino acid sequence identical to that of a S. cerevisiae PDC5 polypeptide; the PDC polypeptide has at least 75% identity to SEQ ID NO:79 (S.c. PDC5); the PDC polypeptide has at least 95% identity to SEQ ID NO:79 (S.c. PDC5); the PDC polypeptide has an amino acid sequence identical to that of a S. cerevisiae PDC6 polypeptide; the PDC polypeptide has at least 75% identity to SEQ ID NO:81 (S.c. PDC6); the PDC polypeptide has at least 95% identity to SEQ ID NO:81 (S.c. PDC6); the PDC polypeptide has an amino acid sequence identical to a PDC polypeptide in FIG. 31; the PDC polypeptide has at least 75% identity to a PDC polypeptide in FIG. 31; the PDC polypeptide has at least 95% identity to a PDC polypeptide in FIG. 31.

In some cases, the modification to increase or decrease organic acid transport activity comprises at least one modification selected from the group consisting of a modification to increase or decrease any of S. pombe Mae1, S. cerevisiae JEN1, K. lactis JEN1, K. lactis JEN2, S. cereale ALMT1, B. napus ALMT1, M. musculus NaDC1, Streptococcus bovis malP, A. thaliana AttDT, R. norvegicus NaDC3, H. sapiens Mct1, H. sapiens Mct2 organic acid transport polypeptide activity or increase or decrease A. oryzae organic acid transporter activity. The genetic modification increases or decreases organic acid transport activity by increasing or decreasing expression of an organic acid transport polypeptide; the genetic modification that increases expression is the addition of a gene encoding an organic acid transport polypeptide; the genetic modification that increases expression is a genetic modification that increases the transcription or translation of a gene encoding an organic acid transport polypeptide; the genetic modification that decreases expression is the deletion of all or part of a gene encoding an organic acid transport polypeptide or the disruption of a gene encoding an organic acid transport polypeptide; the organic acid transport polypeptide is heterologous to the fungal cell; the organic acid transport polypeptide has an amino acid sequence identical to that of S. pombe Mae1; the organic acid transport polypeptide has at least 75% identity to SEQ ID NO:43 (S. pombe Mae1); the organic acid transport polypeptide has at least 95% identity to SEQ ID NO:43 (S. pombe Mae1); the organic acid transport polypeptide has an amino acid sequence identical to that of K. lactis JEN1; the organic acid transport polypeptide has at least 75% identity to SEQ ID NO:25 (K. lactis JEN1); the organic acid transport polypeptide has at least 95% identity to SEQ ID NO:25 (K. lactis JEN1); the organic acid transport polypeptide has an amino acid sequence identical to that of S. cerevisiae JEN1; the organic acid transport polypeptide has at least 75% identity to SEQ ID NO:29 (S. cerevisiae JEN1); the organic acid transport polypeptide has at least 95% identity to SEQ ID NO:29 (S. cerevisiae JEN1); the organic acid transport polypeptide has an amino acid sequence identical to that of K. lactis JEN2; the organic acid transport polypeptide has at least 75% identity to SEQ ID NO:27 (K. lactis JEN2); the organic acid transport polypeptide has at least 95% identity to SEQ ID NO:27 (K. lactis JEN2); the organic acid transport polypeptide has an amino acid sequence identical to that of S. cereale ALMT1; the organic acid transport polypeptide has at least 75% identity to SEQ ID NO:47 (S. cereale ALMT1); the organic acid transport polypeptide has at least 95% identity to SEQ ID NO:47 (S. cereale ALMT1); the organic acid transport polypeptide has an amino acid sequence identical to that of B. napus ALMT1; the organic acid transport polypeptide has at least 75% identity to SEQ ID NO:45 (B. napus ALMT1); the organic acid transport polypeptide has at least 95% identity to SEQ ID NO:45 (B. napus ALMT1); the organic acid transport polypeptide has an amino acid sequence identical to that of M. musculus NaDC1; the organic acid transport polypeptide has at least 75% identity to SEQ ID NO:31 (M. musculus NaDC1); the organic acid transport polypeptide has at least 95% identity to SEQ ID NO:31 (M. musculus NaDC1); the organic acid transport polypeptide has an amino acid sequence identical to that of Streptococcus bovis malP; the organic acid transport polypeptide has at least 75% identity to SEQ ID NO:33 (Streptococcus bovis malP); the organic acid transport polypeptide has at least 95% identity to SEQ ID NO:33 (Streptococcus bovis malP); the organic acid transport polypeptide has an amino acid sequence identical to that of A. thaliana AttDT; the organic acid transport polypeptide has at least 75% identity to SEQ ID NO:35 (A. thaliana AttDT); the organic acid transport polypeptide has at least 95% identity to SEQ ID NO:35 (A. thaliana AttDT); the organic acid transport polypeptide has an amino acid sequence identical to that of R. norvegicus NaDC3; the organic acid transport polypeptide has at least 75% identity to SEQ ID NO:37 (R. norvegicus NaDC3); the organic acid transport polypeptide has at least 95% identity to SEQ ID NO:37 (R. norvegicus NaDC3); the organic acid transport polypeptide has an amino acid sequence identical to that of H. sapiens Mct1; the organic acid transport polypeptide has at least 75% identity to SEQ ID NO:39 (H. sapiens Mct1); the organic acid transport polypeptide has at least 95% identity to SEQ ID NO:39 (H. sapiens Mct1); the organic acid transport polypeptide has an amino acid sequence identical to that of H. sapiens Mct2; the organic acid transport polypeptide has at least 75% identity to SEQ ID NO:41 (H. sapiens Mct2); the organic acid transport polypeptide has at least 95% identity to SEQ ID NO:41 (H. sapiens Mct2); the organic acid transport polypeptide has an amino acid sequence identical to an organic acid transport polypeptide in FIG. 35; the organic acid transport polypeptide has at least 75% identity to an organic acid transport polypeptide in FIG. 35; the organic acid transport polypeptide has at least 95% identity to an organic acid transport polypeptide in FIG. 35; the organic acid transporter is identical to SEQ ID NO______ (A. oryzae organic acid transporter); the organic acid transport polypeptide has at least 75% identity to SEQ ID NO______ (A. oryzae organic acid transporter); the organic acid transport polypeptide has at least 95% identity to SEQ ID NO______ (A. oryzae organic acid transporter); the organic acid transporter is identical to an organic acid transporter polypeptide in FIG. 62; the organic acid transport polypeptide has at least 75% identity to an organic acid transport polypeptide in FIG. 62; the organic acid transport polypeptide has at least 95% identity to an organic acid transport polypeptide in FIG. 62. A. oryzae

In some cases, the modification to increase or decrease glucose sensing and regulatory polypeptide activity comprises at least one modification selected from the group consisting of modifications to increase or decrease SNF1, MIG1, MIG2, HXK2, RGT1, SNF3, RGT2, STD1, MTH1, GRR1, YCK1, HXK1, and GLK1 polypeptide activity; the genetic modification increases or decreases glucose sensing and regulatory polypeptide activity by increasing or decreasing expression of a glucose sensing and regulatory polypeptide; the genetic modification that increases expression is the addition of a gene encoding a glucose sensing and regulatory polypeptide; the genetic modification that increases expression is a genetic modification that increases the transcription or translation of a gene encoding a glucose sensing and regulatory polypeptide; the genetic modification that decreases expression is the deletion of all or part of a gene encoding a glucose sensing and regulatory polypeptide or the disruption of a gene encoding a glucose sensing and regulatory polypeptide; the glucose sensing and regulatory polypeptide is heterologous to the fungal cell; the glucose sensing and regulatory polypeptide has an amino acid sequence identical to that of SNF1; the glucose sensing and regulatory polypeptide has at least 75% identity to SEQ ID NO:107 (S. cerevisiae SNF1); the glucose sensing and regulatory polypeptide has at least 95% identity to SEQ ID NO: 107 (S. cerevisiae SNF1); the glucose sensing and regulatory polypeptide has an amino acid sequence identical to that of MIG1; the glucose sensing and regulatory polypeptide has at least 75% identity to SEQ ID NO:109 (S. cerevisiae MIG1); the glucose sensing and regulatory polypeptide has at least 95% identity to SEQ ID NO:109 (S. cerevisiae MIG1); the glucose sensing and regulatory polypeptide has an amino acid sequence identical to that of MIG2; the glucose sensing and regulatory polypeptide has at least 75% identity to SEQ ID NO:111 (S. cerevisiae MIG2); the glucose sensing and regulatory polypeptide has at least 95% identity to SEQ ID NO:111 (S. cerevisiae MIG2); the glucose sensing and regulatory polypeptide has an amino acid sequence identical to that of HXK2; the glucose sensing and regulatory polypeptide has at least 75% identity to SEQ ID NO:113 (S. cerevisiae HXK2); the glucose sensing and regulatory polypeptide has at least 95% identity to SEQ ID NO:113 (S. cerevisiae HXK2); the glucose sensing and regulatory polypeptide has an amino acid sequence identical to that of RGT1; the glucose sensing and regulatory polypeptide has at least 75% identity to SEQ ID NO:115 (S. cerevisiae RGT1); the glucose sensing and regulatory polypeptide has at least 95% identity to SEQ ID NO:115 (S. cerevisiae RGT1); the glucose sensing and regulatory polypeptide has an amino acid sequence identical to that of SNF3; the glucose sensing and regulatory polypeptide has at least 75% identity to SEQ ID NO:117 (S. cerevisiae SNF3); the glucose sensing and regulatory polypeptide has at least 95% identity to SEQ ID NO:117 (S. cerevisiae SNF3); the glucose sensing and regulatory polypeptide has an amino acid sequence identical to that of RGT2; the glucose sensing and regulatory polypeptide has at least 75% identity to SEQ ID NO:119 (S. cerevisiae RGT2); the glucose sensing and regulatory polypeptide has at least 95% identity to SEQ ID NO:119 (S. cerevisiae RGT2); the glucose sensing and regulatory polypeptide has an amino acid sequence identical to that of STD1; the glucose sensing and regulatory polypeptide has at least 75% identity to SEQ ID NO:121 (S. cerevisiae STD1); the glucose sensing and regulatory polypeptide has at least 95% identity to SEQ ID NO:121 (S. cerevisiae STD1); the glucose sensing and regulatory polypeptide has an amino acid sequence identical to that of MTH1; the glucose sensing and regulatory polypeptide has at least 75% identity to SEQ ID NO:145 (S. cerevisiae MTH1); the glucose sensing and regulatory polypeptide has at least 95% identity to SEQ ID NO:145 (S. cerevisiae MTH1); the glucose sensing and regulatory polypeptide has an amino acid sequence identical to that of MTH1ΔTAM; the glucose sensing and regulatory polypeptide has at least 75% identity to SEQ ID NO:147 (S. cerevisiae MTH1ΔTAM); the glucose sensing and regulatory polypeptide has at least 95% identity to SEQ ID NO:147 (S. cerevisiae MTH1Δ TAM); the glucose sensing and regulatory polypeptide has an amino acid sequence identical to that of GRR1; the glucose sensing and regulatory polypeptide has at least 75% identity to SEQ ID NO:123 (S. cerevisiae GRR1); the glucose sensing and regulatory polypeptide has at least 95% identity to SEQ ID NO:123 (S. cerevisiae GRR1); the glucose sensing and regulatory polypeptide has an amino acid sequence identical to that of YCK1; the glucose sensing and regulatory polypeptide has at least 75% identity to SEQ ID NO:125 (S. cerevisiae YCK1); the glucose sensing and regulatory polypeptide has at least 95% identity to SEQ ID NO:125 (S. cerevisiae YCK1); the glucose sensing and regulatory polypeptide has an amino acid sequence identical to that of HXK1; the glucose sensing and regulatory polypeptide has at least 75% identity to SEQ ID NO:127 (S. cerevisiae HXK1); the glucose sensing and regulatory polypeptide has at least 95% identity to SEQ ID NO:127 (S. cerevisiae HXK1); the glucose sensing and regulatory polypeptide has an amino acid sequence identical to that of GLK1; the glucose sensing and regulatory polypeptide has at least 75% identity to SEQ ID NO:129 (S. cerevisiae GLK1); the glucose sensing and regulatory polypeptide has at least 95% identity to SEQ ID NO:129 (S. cerevisiae GLK1); the glucose sensing and regulatory polypeptide has an amino acid sequence identical to a glucose sensing and regulatory polypeptide in any of FIGS. 50-61; the glucose sensing and regulatory polypeptide has at least 75% identity to a glucose sensing and regulatory polypeptide in FIGS. 50-61; the glucose sensing and regulatory polypeptide has at least 95% identity to a glucose sensing and regulatory polypeptide in FIGS. 50-61.

In some cases, the modification to increase hexose transporter (HXT) activity comprises at least one modification selected from the group consisting of modifications to increase or decrease HXT1, HXT2, HXT3, HXT3, HXT4, HXT5, HXT6, or HXT7 polypeptide activity; the genetic modification increases or decreases HXT activity by increasing or decreasing expression of a HXT polypeptide; the genetic modification that increases expression is the addition of a gene encoding a HXT polypeptide; the genetic modification that increases expression is a genetic modification that increases the transcription or translation of a gene encoding a HXT polypeptide; the genetic modification that decreases expression is the deletion of all or part of a gene encoding a HXT polypeptide or the disruption of a gene encoding HXT polypeptide; the HXT polypeptide is heterologous to the fungal cell; the HXT polypeptide has an amino acid sequence identical to that of S. cerevisiae HXT1; the HXT polypeptide has at least 75% identity to SEQ ID NO:131 (S. cerevisiae HXT1); the HXT polypeptide has at least 95% identity to SEQ ID NO:131 (S. cerevisiae HXT1); the HXT polypeptide has an amino acid sequence identical to that of S. cerevisiae HXT2; the HXT polypeptide has at least 75% identity to SEQ ID NO:133 (S. cerevisiae HXT2); the HXT polypeptide has at least 95% identity to SEQ ID NO:133 (S. cerevisiae HXT2); the HXT polypeptide has an amino acid sequence identical to that of S. cerevisiae HXT3; the HXT polypeptide has at least 75% identity to SEQ ID NO:135 (S. cerevisiae HXT3); the HXT polypeptide has at least 95% identity to SEQ ID NO:135 (S. cerevisiae HXT3); the HXT polypeptide has an amino acid sequence identical to that of S. cerevisiae HXT4; the HXT polypeptide has at least 75% identity to SEQ ID NO:137 (S. cerevisiae HXT4); the HXT polypeptide has at least 95% identity to SEQ ID NO:137 (S. cerevisiae HXT4); the HXT polypeptide has an amino acid sequence identical to that of S. cerevisiae HXT5; the HXT polypeptide has at least 75% identity to SEQ ID NO:139 (S. cerevisiae HXT5); the HXT polypeptide has at least 95% identity to SEQ ID NO:139 (S. cerevisiae HXT5); the HXT polypeptide has an amino acid sequence identical to that of S. cerevisiae HXT6);: HXT polypeptide has at least 75% identity to SEQ ID NO:141 (S. cerevisiae HXT6); the HXT polypeptide has at least 95% identity to SEQ ID NO:141 (S. cerevisiae HXT6); the HXT polypeptide has an amino acid sequence identical to that of S. cerevisiae HXT7; the HXT polypeptide has at least 75% identity to SEQ ID NO:143 (S. cerevisiae HXT7); the HXT polypeptide has at least 95% identity to SEQ ID NO:143 (S. cerevisiae HXT7); the HXT polypeptide has an amino acid sequence identical to a hexose transporter (HXT) polypeptide in FIG. 44; the HXT polypeptide has at least 75% identity to a hexose transporter (HXT) polypeptide in FIG. 44; the HXT polypeptide has at least 95% identity to a hexose transporter (HXT) polypeptide in FIG. 44.

In some cases, the recombinant fungal cell comprises more than one modification selected from the group consisting of modifications to: a) increase anaplerotic activity; b) decrease PDC activity; c) increase or decrease organic acid transport activity; d) increase or decrease glucose sensing and regulatory polypeptide activity; e) increase hexose transporter (HXT) activity; and f) increase or decrease C4 dicarboxylic acid biosynthetic activity.

In some cases, the more than one modification are selected from the group consisting of modifications to: a) increase anaplerotic activity; b) decrease PDC activity; c) increase organic acid transport activity; d) increase glucose sensing and regulatory polypeptide activity; and e) increase C4 dicarboxylic acid biosynthetic activity.

In some cases, the modification to increases anaplerotic activity is one or more modification selected from the group of modifications to increase PYC, PPC, or PCK activity; the modification to increases C4 dicarboxylic acid biosynthetic activity is one or more modification selected from the group of modifications to increase MDH, fumarase, or fumarate reductase activity; the modification to decrease PDC activity is one or more modifications selected from the group of modifications to decrease the activity of one or more of PDC1, PDC5, or PDC6 polypeptides; and the modification to increase organic acid transport activity is one or more modification selected from the group of modifications to increase the activity of one or more of S. pombe Mae1, S. cereale ALMT1, B. napus ALMT1 polypeptides, and an A. oryzae organic acid transporter.

In some cases, the more than one modifications are selected from the group consisting of modifications to: a) increase PYC1 or PYC2 activity; b) increase MDH2 or MDH3 activity; d) decrease each of PDC1, PDC5, and PDC6 activities; d) increase S. pombe MAE1 (malic acid transporter) activity; e) increase A. oryzae organic acid transporter activity; and f) increase or decrease MTH1 activity.

In some cases the modifications include modification to increase PDC1 activity and/or increase PDC6 activity and decrease pdc5 activity. Thus, a desirable strain can lack an active pdc1 gene and harbor one or both of a heterologous PDC6 gene and PDC5 genes.

In some cases, the more than one modifications are selected from the group consisting of modifications to: a) increase PYC1 or PYC2 activity; b) increase MDH2 or MDH3 activity; c) decrease each of PDC1 and PDC5 activities; d) increase S. pombe MAE1 (malic acid transporter) activity; e) increase an A. oryzae organic acid transporter. and f) increase or decrease MTH1 activity.

In some cases, the more than one modifications are selected from the group consisting of modifications to: a) increase PYC1 or PYC2 activity; b) increase MDH2 or MDH3 activity; c) decrease each of PDC1, PDC5, and PDC6 activities; d) increase fumarase activity; and e) increase or decrease MTH1 activity.

In some cases, the more than one modifications are selected from the group consisting of modifications to: a) increase PYC1 or PYC2 activity; b) increase MDH2 or MDH3 activity; c) decrease each of PDC1 and PDC5 activities; d) increase fumarase activity; and e) increase or decrease MTH1 activity.

In some cases, the more than one modifications are selected from the group consisting of modifications to: a) increase PYC1 or PYC2 activity; b) increase MDH2 or MDH3 activity; c) decrease each of PDC1, PDC5, and PDC6 activities; d) increase fumarase activity; e) increase fumarate reductase activity; and f) increase or decrease MTH1 activity.

In some cases, the more than one modifications are selected from the group consisting of modifications to: a) increase PYC1 or PYC2 activity; b) increase MDH2 or MDH3 activity; c) decrease each of PDC1 and PDC5 activities; d) increase fumarase activity; e) increase fumarate reductase activity; and f) increase or decrease MTH1 activity.

In some cases, the modification to increase fumarase activity is comprised of a modification to increase FUM1 polypeptide activity; and the modification to increase fumarate reductase activity is selected from the group consisting of modifications to increase OSM1 or FRDS1 polypeptide activity.

In various cases, the fungal cell comprises more than two modifications selected from the group consisting of modifications to: a) increase anaplerotic activity; b) decrease PDC activity; c) increase or decrease organic acid transport activity; d) increase or decrease glucose sensing and regulatory polypeptide activity; e) increase HXT activity; and f) increase or decrease C4 dicarboxylic acid biosynthetic activity.

In some cases: the fungal cell is of a genus selected from the group consisting of Saccharomyces, Zygosaccharomyces, Yarrowia, Kluyveromyces, Aspergillus, or Pichia spp; the fungal cell is Saccharomyces cerevisiae; the Saccharomyces cerevisiae is TAM, Lp4f, m850, RWB837, or derivatives thereof; the fungal cell is Saccharomyces bayanus, Saccharomyces cerevisiae var bayanus, or Saccharomyces boulardii; the fungal cell is Kluyveromyces lactis; the fungal cell is Aspergillus niger; the fungal cell is Yarrowia lipolytica.

Also disclosed is a method of producing a C4-dicarboxylic acid, comprising: culturing a recombinant fungal cell described herein under conditions that achieve C4-dicarboxylic acid production.

In various cases: the method further includes isolating a produced C4-dicarboxylic acid; the C4-dicarboxylic acid is selected from the group consisting of malic acid, fumaric acid, tartaric acid, and succinic acid; the step of culturing under conditions that achieve C4-dicarboxylic acid production comprises culturing at a pH within the range of 1.5 to 7; the pH is lower than 5.0; pH is lower than 4.5; the pH is lower than 4.0; the pH is lower than 3.5; the pH is lower than 3.0; the pH is lower than 2.5; the pH is lower than 2.0; the step of culturing under conditions that achieve C4-dicarboxylic acid production comprises culturing under conditions and for a time sufficient for C4-dicarboxylic acid to accumulate to a level within the range of 10 to 200 g/L; the C4-dicarboxylic acid is selected from the group consisting of malic acid, fumaric acid, tartaric acid, and succinic acid; the C4-dicarboxylic acid accumulates to greater than 30 g/L (greater than 50 g/L; greater than 75 g/L; greater than 100 g/L; greater than 125 g/L; or greater than 150 g/L). In other cases the pH is allowed to decrease by at least 1, at least 2, at least 3 pH units during culturing. Thus, the pH can decrease below 5, below 4 or below 3 during culturing after starting at a higher pH.

In various cases: the step of culturing under conditions that achieve C4-dicarboxylic acid production comprises culturing under conditions and for a time sufficient for C4-dicarboxylic acid to accumulate to a level within a range of about 0.3 moles of C4-dicarboxylic acid per mole of substrate to about 1.75 moles of C4-dicarboxylic acid per mole of substrate; the C4-dicarboxylic acid is selected from the group consisting of malic acid, fumaric acid, tartaric acid, and succinic acid; the C4-dicarboxylic acid accumulates to greater than about 0.3 moles of C4-dicarboxylic acid per mole of substrate; the C4-dicarboxylic acid accumulates to greater than about 0.5 moles of C4-dicarboxylic acid per mole of substrate; the C4-dicarboxylic acid accumulates to greater than about 0.75 moles of C4-dicarboxylic acid per mole of substrate; the C4-dicarboxylic acid accumulates to greater than about 1.0 moles of C4-dicarboxylic acid per mole of substrate; the C4-dicarboxylic acid accumulates to greater than about 1.25 moles of C4-dicarboxylic acid per mole of substrate; the C4-dicarboxylic acid accumulates to greater than about 1.5 moles of C4-dicarboxylic acid per mole of substrate; the C4-dicarboxylic acid accumulates to greater than about 1.75 moles of C4-dicarboxylic acid per mole of substrate; the substrate is glucose; the step of culturing under conditions that achieve C4-dicarboxylic acid production comprises culturing in a medium comprising a carbon source; the carbon source is one or more carbon sources selected from the group consisting of glucose, glycerol, sucrose, fructose, maltose, lactose, galactose, hydrolyzed starch, corn syrup, high fructose corn syrup, and hydrolyzed lignocelluloses; the carbon source is glucose; the medium further comprises a carbon dioxide source; the carbon dioxide source comprises calcium carbonate or carbon dioxide gas; the carbon dioxide source is calcium carbonate; the carbon dioxide source is carbon dioxide gas.

Also disclosed is a method of preparing a food or feed additive containing a C4-dicarboxylic acid, the method comprising steps of: a) cultivating the recombinant fungal cell described herein under conditions that allow production of the C4-dicarboxylic acid; b) isolating the C4-dicarboxylic acid; and c) combining the isolated C4-dicarboxylic acid with one or more other food or feed additive components. In various cases: the C4-dicarboxylic acid is selected from the group consisting of malic acid, fumaric acid, tartaric acid, and succinic acid.

Also disclosed is a method of preparing a cosmetic containing a C4-dicarboxylic acid, the method comprising steps of: a) cultivating a recombinant fungal cell described herein under conditions that allow production of the C4-dicarboxylic acid; b) isolating the C4-dicarboxylic acid; and c) combining the C4-dicarboxylic acid with one or more cosmetic components. In various cases, the C4-dicarboxylic acid is selected from the group consisting of malic acid, fumaric acid, tartaric acid, and succinic acid.

Also described is a method of preparing an industrial chemical containing a C4-dicarboxylic acid, the method comprising steps of: a) cultivating the recombinant fungal cell described herein under conditions that allow production of the C4-dicarboxylic acid; b) isolating the C4-dicarboxylic acid; and c) combining the isolated C4-dicarboxylic acid with one or more industrial chemical components. In various cases, the C4-dicarboxylic acid is selected from the group consisting of malic acid, fumaric acid, tartaric acid, and succinic acid.

Also disclosed is a method of preparing a biodegradable polymer containing a C4-dicarboxylic acid, the method comprising steps of: a) cultivating the recombinant fungal cell described here under conditions that allow production of the C4-dicarboxylic acid; b) isolating the C4-dicarboxylic acid; and c) combining the isolated C4-dicarboxylic acid with one or more biodegradable polymer components. In various cases, the C4-dicarboxylic acid is selected from the group consisting of malic acid, fumaric acid, tartaric acid, and succinic acid.

Also disclosed is a method of preparing a C4-dicarboxylic acid derivative, the method comprising steps of: a) cultivating the recombinant fungal cell described herein under conditions that allow production of a C4-dicarboxylic acid; b) isolating the C4-dicarboxylic acid; and c) converting the isolated C4-dicarboxylic acid into a C4-dicarboxylic acid derivative. In various cases: the C4-dicarboxylic acid is chosen from one or more of malic acid, fumaric acid, tartaric acid, and succinic acid; the C-4 dicarboxylic acid derivative is chosen from one or more of: tetrahydrofuran (THF), butane diol (e.g. 1,4-butanediol), γ-butyrolactone, pyrrolidinones (e.g. N-methyl-2-Pyrrolidone), esters, diamines, 4,4-Bionelle, hydroxybutyric acid, dibasic ester (DBE), succindiamide, 1,4-diaminobutane, succinonitrile, maleic anhydride, a hydroxybutyrolactone derivative, a hydroxysuccinate derivative and an unsaturated succinate derivative; the converting comprises one or more of physical treatments, fermentation, biocatalysis, and chemical transformation; the converting comprises one or more physical treatments; the converting comprises fermentation; the converting comprises one or more chemical transformations; the converting comprises one or more biocatalysis.

The fermenatation methods can include liquid fermentation and solid state fermentation (Krishna 2005 Crit Rev Biotechnol 25:1)

### BRIEF DESCRIPTION OF THE DRAWINGS

FIG. 1 shows glucose and pyruvate concentrations as a function of culture time as described in Example 1.

FIG. 2 shows malate, glycerol, and succinate concentrations as a function of culture time as described in Example 1.

FIG. 3 is a map of plasmid p426GPDMDH3, as described in Example 1.

FIG. 4 is a map of plasmid pRS2, as described in Example 1.

FIG. 5 is a map of plasmid pRS2ΔMDH3, as described in Example 1.

FIG. 6 is a map of plasmid YEplac112 SpMAE1, as described in Example 1.

FIG. 7 shows the biomass, the consumption of glucose, and the production of pyruvate in Batch A, Example 2.

FIG. 8 shows the production of malate, glycerol, and succinate in Batch A, Example 2.

FIG. 9 shows the biomass, the consumption of glucose, and the production of pyruvate in Batch B, Example 2.

FIG. 10 shows the production of malate, glycerol, and succinate in Batch B, Example 2.

FIG. 11 shows the biomass, the consumption of glucose, and the production of pyruvate in Batch C, Example 2.

FIG. 12 shows the production of malate, glycerol, and succinate in Batch C, Example 2.

FIG. 13 is a table with enzyme activities in yeast strains expressing E. coli ppc.

FIG. 14 is a table with enzyme activities in yeast strains overexpressing MDH2 and E. coli ppc.

FIG. 15 is a table with various metabolite levels of yeast strains expressing E. coli ppc and grown in shake flasks with 2% or 10% glucose as carbon source.

FIG. 16 is a table with various metabolite levels of yeast strains overexpressing MDH2 and E. coli ppc and grown in shake flasks with 2% or 10% glucose as carbon source.

FIG. 17 is a table with intracellular malate concentrations of yeast strains overexpressing MDH2 and E. coli ppc when grown on mineral medium with 2% glucose.

FIG. 18 is a table of malate dehydrogenase activities of wild-type yeast in the presence and absence of MDH1ΔL and MDH3ΔSKL plasmids.

FIG. 19 is a table with various metabolite levels of yeast strains with one or more genetic modifications.

FIG. 20 is a table with various enzyme activities of yeast in the presence and absence of MDH1ΔL and MDH3ΔSKL plasmids.

FIG. 21 is a table with various metabolite levels of yeast strains with one or more genetic modifications.

FIG. 22 shows the effect of various inhibitors on wild-type E. coli PEP carboxylase activity.

FIG. 23 shows the effect of various inhibitors on mutant E. coli PEP carboxylase activity.

FIG. 24 is a table with various metabolite levels of yeast strains with one or more genetic modifications.

FIG. 25 shows fermentation results from PDC6/pdc6 and pdc6/pdc6 diploid strains.

FIG. 26 shows fermentation results from PDC6 and pdc6 haploid strains.

FIG. 27 shows fermentation results from strains expressing a Mdh2 (P2S) variant protein.

FIGS. 28a-f depict organic acids (malic acid, fumaric acid, succinic acid and tartaric acid) and representative pathways for the production of such organic acid.

FIGS. 29-61 are tables referenced throughout the description. Each reference and information designated by each of the Genbank Accession and GI numbers are hereby incorporated by reference in their entirety. The entries in the tables are organized for convenient reference and the order is not intended to reflect preferences for certain nucleotide or amino acid sequences.

FIG. 29 is a table with amino acid sequences of exemplary proteins for organic acid production in fungal cells.

FIG. 30 is a table with nucleotide sequences encoding exemplary proteins for organic acid production in fungal cells.

FIG. 31 is a table of exemplary pyruvate decarboxylase polypeptides for organic acid production in fungal cells.

FIG. 32 is a table of exemplary phosphoenolpyruvate carboxylase polypeptides for organic acid production in fungal cells.

FIG. 33 is a table of exemplary pyruvate carboxylase polypeptides for organic acid production in fungal cells.

FIG. 34 is a table of exemplary malate dehydrogenase polypeptides for organic acid production in fungal cells.

FIG. 35 is a table of exemplary organic acid transporter polypeptides for organic acid production in fungal cells.

FIG. 36 is a table of exemplary phospoenolpyruvate carboxykinase polypeptides for organic acid production in fungal cells.

FIG. 37 is a table of exemplary malate synthase polypeptides for organic acid production in fungal cells.

FIG. 38 is a table of exemplary malic enzyme polypeptides for organic acid production in fungal cells.

FIG. 39 is a table of exemplary bicarbonate transporter polypeptides for organic acid production in fungal cells.

FIG. 40 is a table of exemplary carbonic anhydrase polypeptides for organic acid production in fungal cells.

FIG. 41a is a table of exemplary ATP citrate lyase subunit 1 polypeptides for organic acid production in fungal cells.

FIG. 41b is a table of exemplary ATP citrate lyase subunit 2 polypeptides for organic acid production in fungal cells.

FIG. 42 is a table of exemplary fumarate reductase polypeptides for organic acid production in fungal cells.

FIG. 43 is a table of exemplary fumarase polypeptides for organic acid production in fungal cells.

FIG. 44 is a table of exemplary hexose transporter polypeptides for organic acid production in fungal cells.

FIG. 45 is a table of exemplary pyruvate kinase polypeptides for organic acid production in fungal cells.

FIG. 46 is a table of exemplary biotin protein ligase polypeptides for organic acid production in fungal cells.

FIG. 47 is a table of exemplary succinate dehydrogenase polypeptides for organic acid production in fungal cells.

FIG. 48 is a table of exemplary vitamin H transporter polypeptides for organic acid production in fungal cells.

FIG. 49 is a table of exemplary isocitrate lyase polypeptides for organic acid production in fungal cells.

FIG. 50 is a table of exemplary glucose sensing and regulatory (Hexokinase) polypeptides for organic acid production in fungal cells.

FIG. 51 is a table of exemplary glucose sensing and regulatory (STD1) polypeptides for organic acid production in fungal cells.

FIG. 52 is a table of exemplary glucose sensing and regulatory (MIG1) polypeptides for organic acid production in fungal cells.

FIG. 53 is a table of exemplary glucose sensing and regulatory (MIG2) polypeptides for organic acid production in fungal cells.

FIG. 54 is a table of exemplary glucose sensing and regulatory (GLK1) polypeptides for organic acid production in fungal cells.

FIG. 55 is a table of exemplary glucose sensing and regulatory (SNF1) polypeptides for organic acid production in fungal cells.

FIG. 56 is a table of exemplary glucose sensing and regulatory (SNF3) polypeptides for organic acid production in fungal cells.

FIG. 57 is a table of exemplary glucose sensing and regulatory (YCK1) polypeptides for organic acid production in fungal cells.

FIG. 58 is a table of exemplary glucose sensing and regulatory (GRR1) polypeptides for organic acid production in fungal cells.

FIG. 59 is a table of exemplary glucose sensing and regulatory (MTH1) polypeptides for organic acid production in fungal cells.

FIG. 60 is a table of exemplary glucose sensing and regulatory (RGT1) polypeptides for organic acid production in fungal cells.

FIG. 61 is a table of exemplary glucose sensing and regulatory (RGT2) polypeptides for organic acid production in fungal cells.

FIG. 62 is a table of exemplary organic acid transporters for organic acid production in fungal cells.

### DEFINITIONS

Accumulation: As used herein, “accumulation” of an organic acid above background levels refers to accumulation to detectable levels. In some cases, “accumulation” refers to accumulation above a pre-determined level (e.g., above a level achieved under otherwise identical conditions with a fungal cell that has not been modified as described herein). In other cases, “accumulation” refers to titer of an organic acid, i.e. grams per liter of one or more organic acids in the broth of a cultured fungal cell. Any available assay, including those explicitly set forth herein, may be used to detect and/or quantify organic acid accumulation.

Amplification: The term “amplification” refers to increasing the number of copies of a desired nucleic acid molecule in a cell. Typically, amplification results in an increased level of activity of polypeptide (e.g., an enzyme) encoded by the nucleic acid molecule, and/or to an increased level of activity of the encoded polypeptide in a desirable location (e.g., in the cytosol).

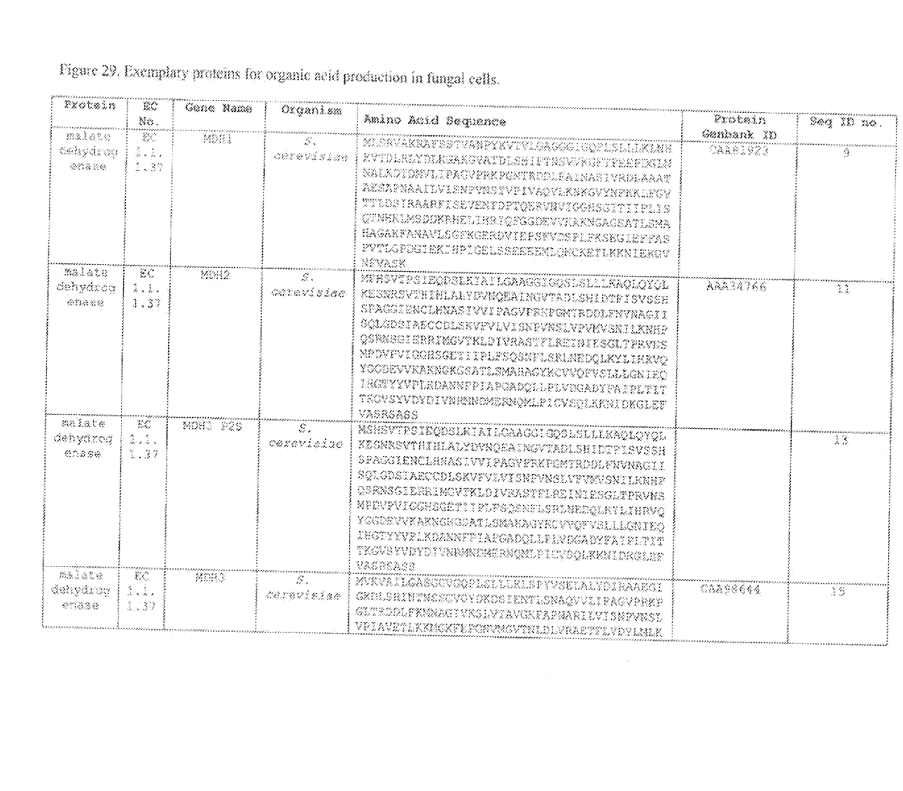

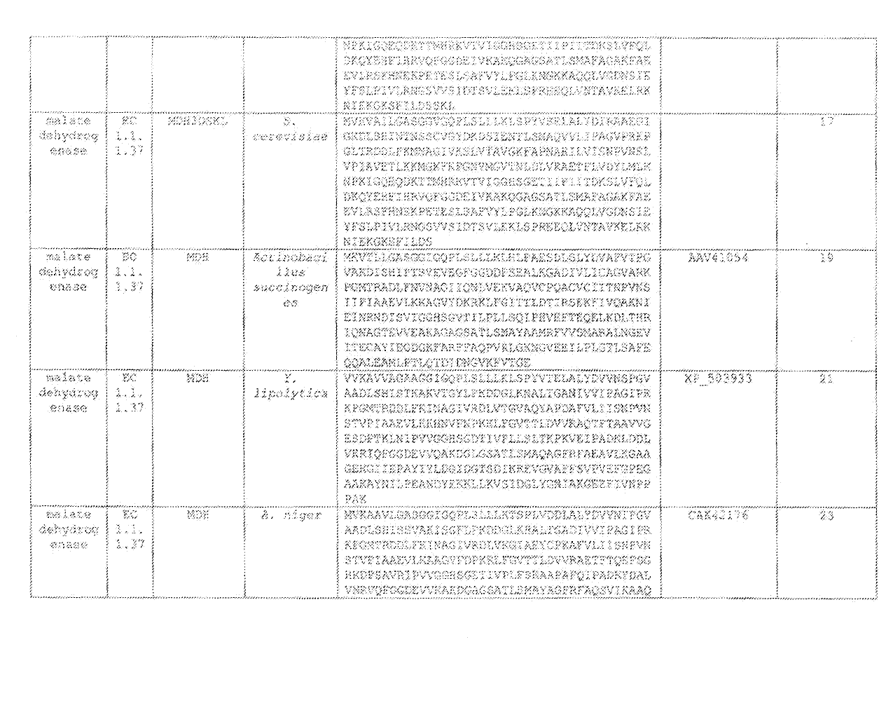

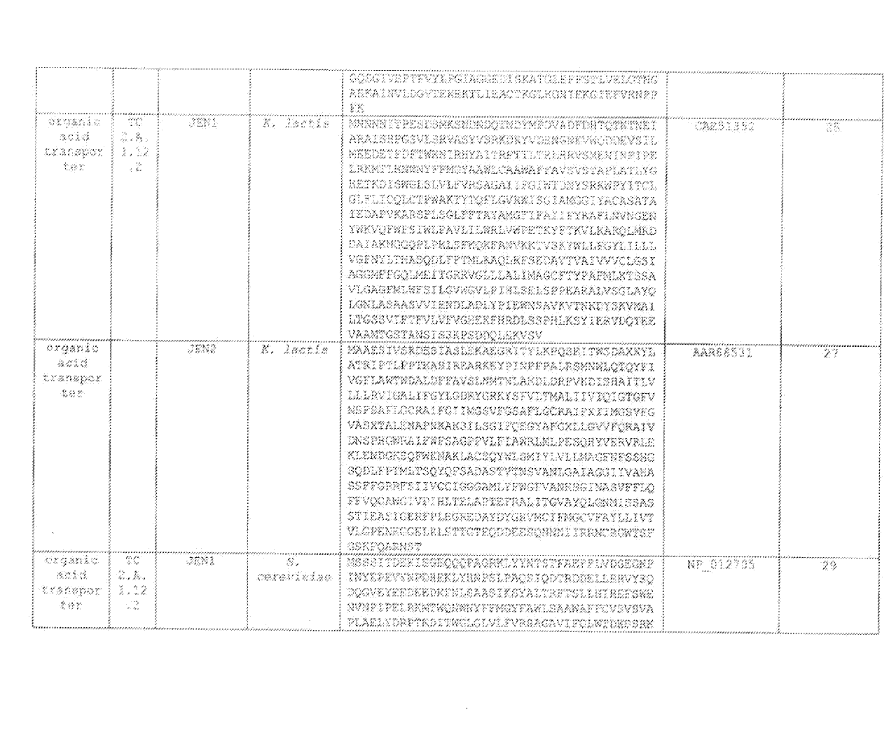

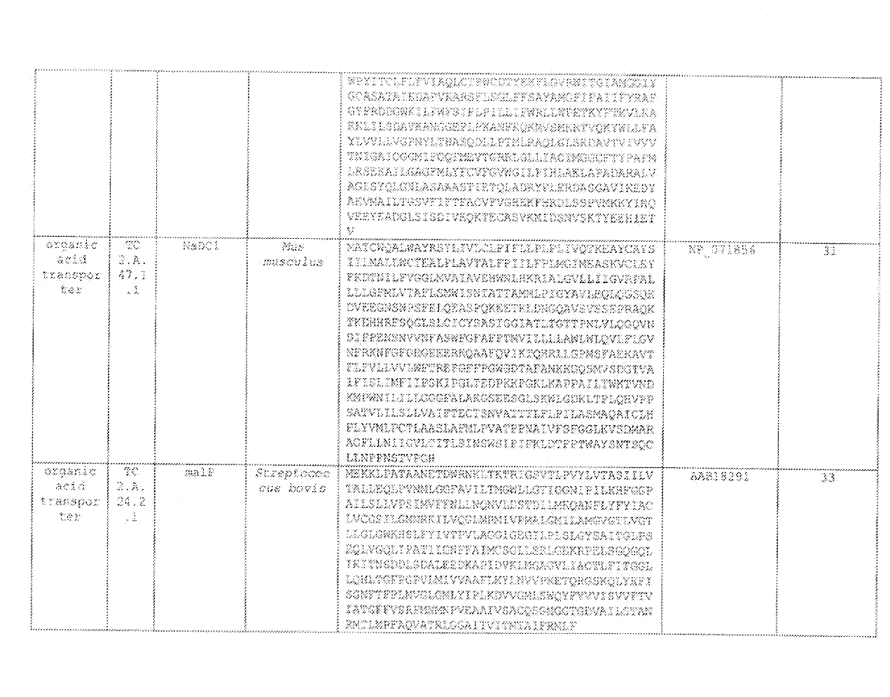

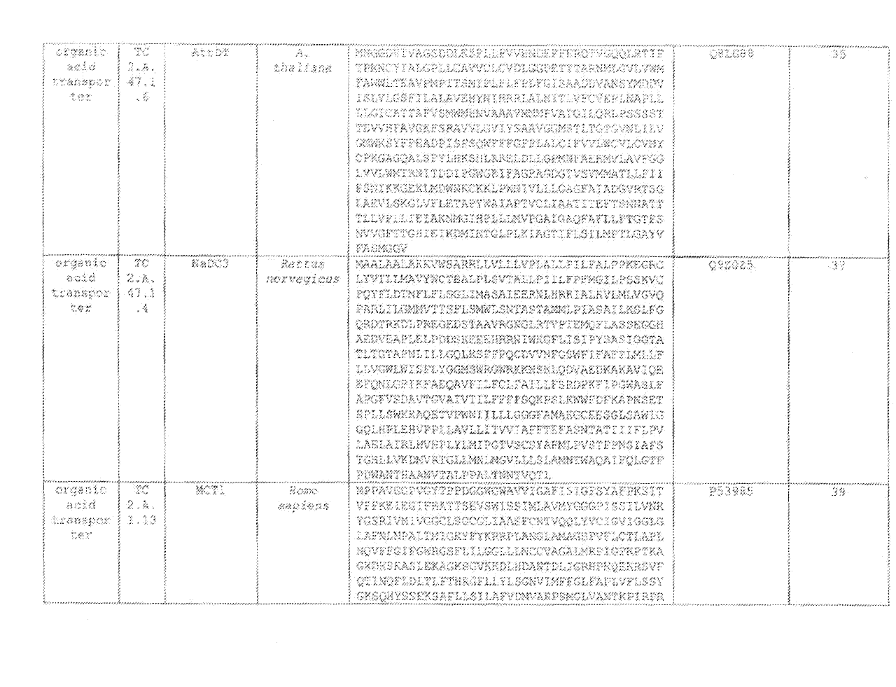

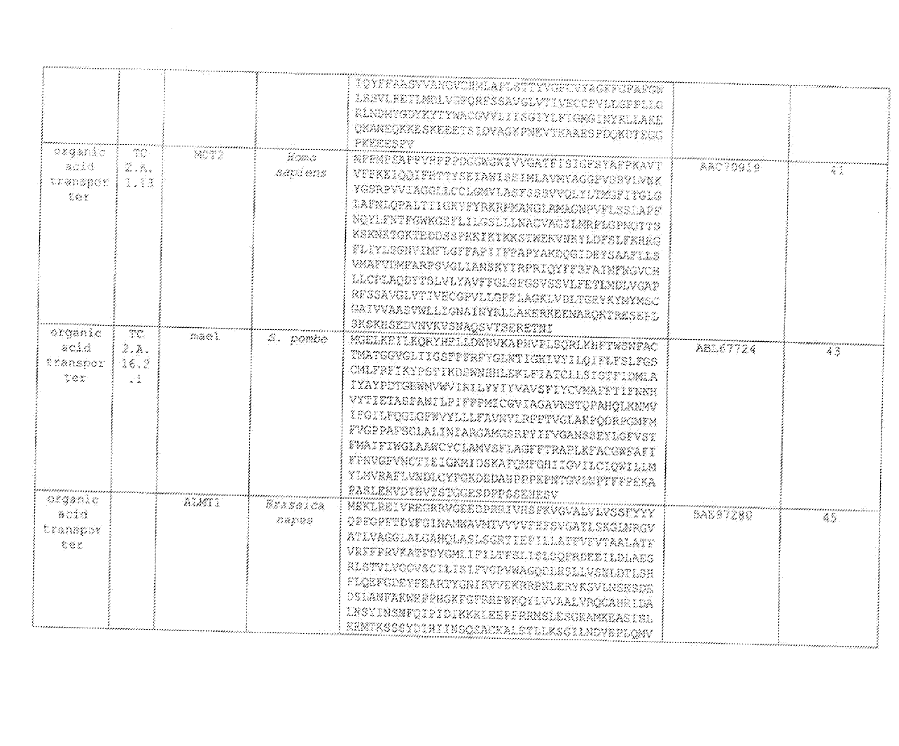

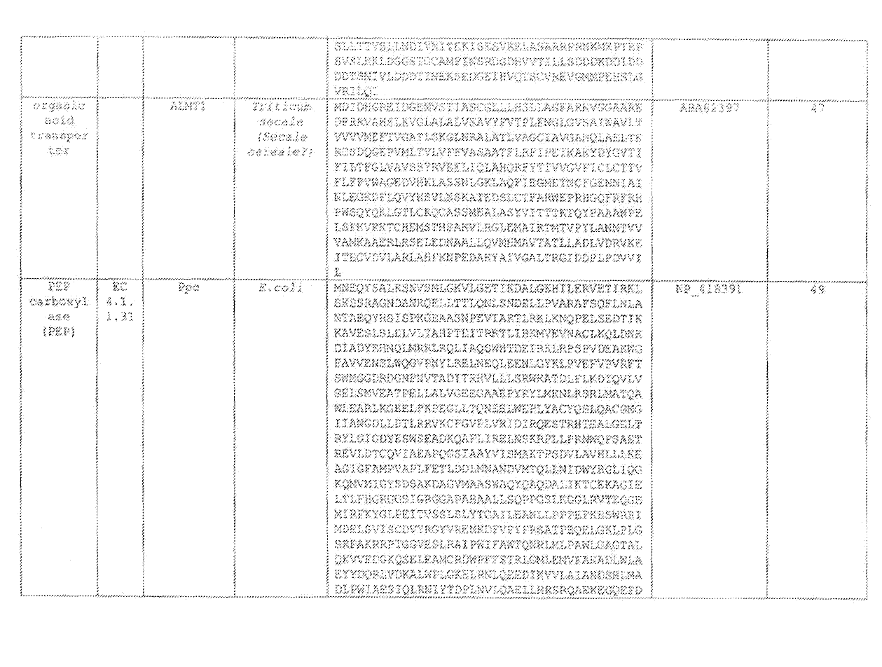

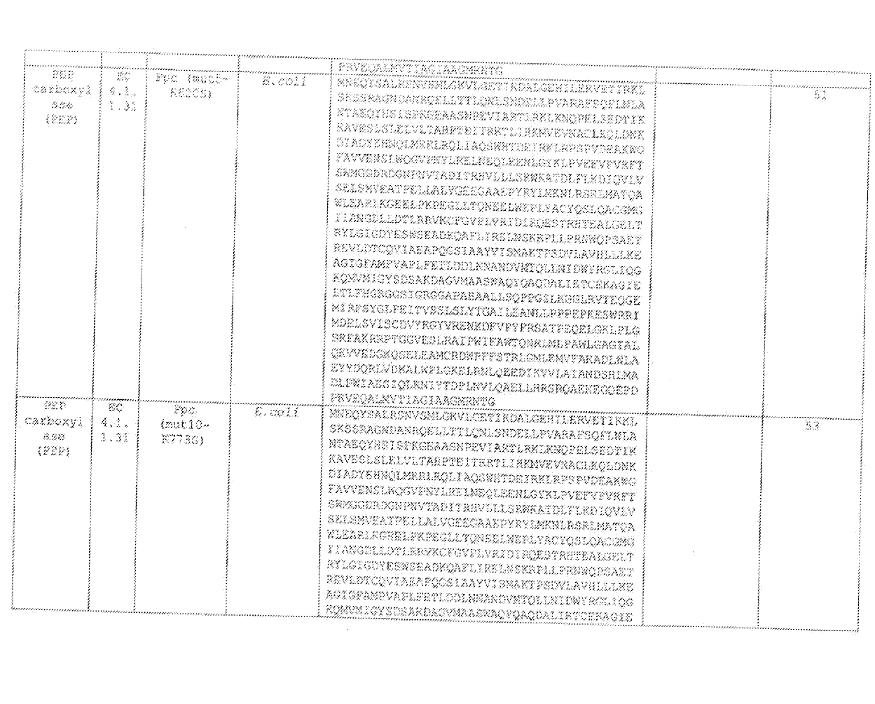

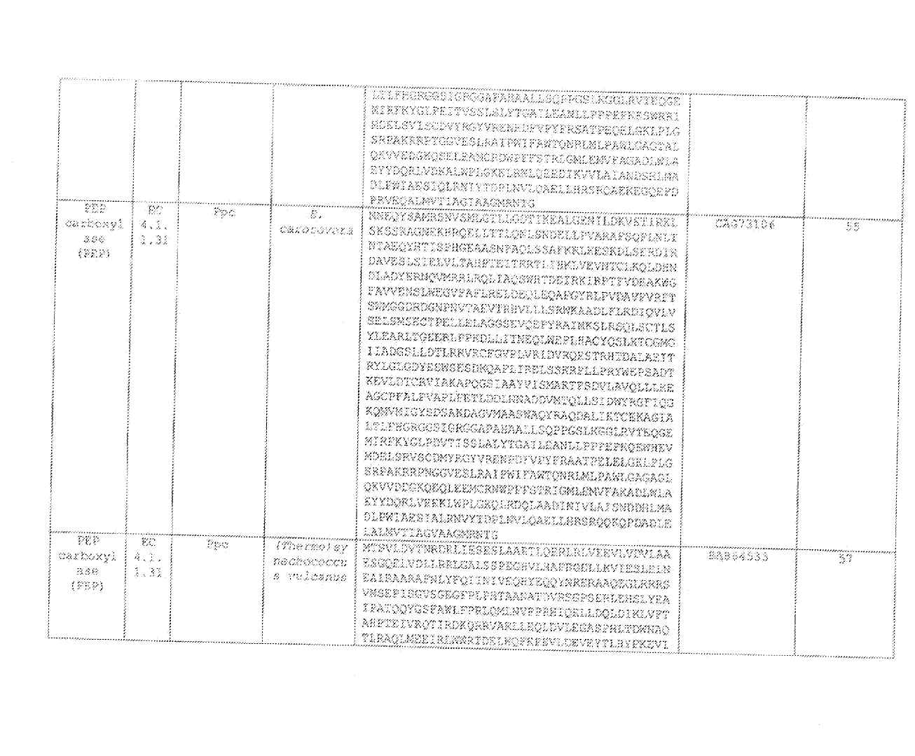

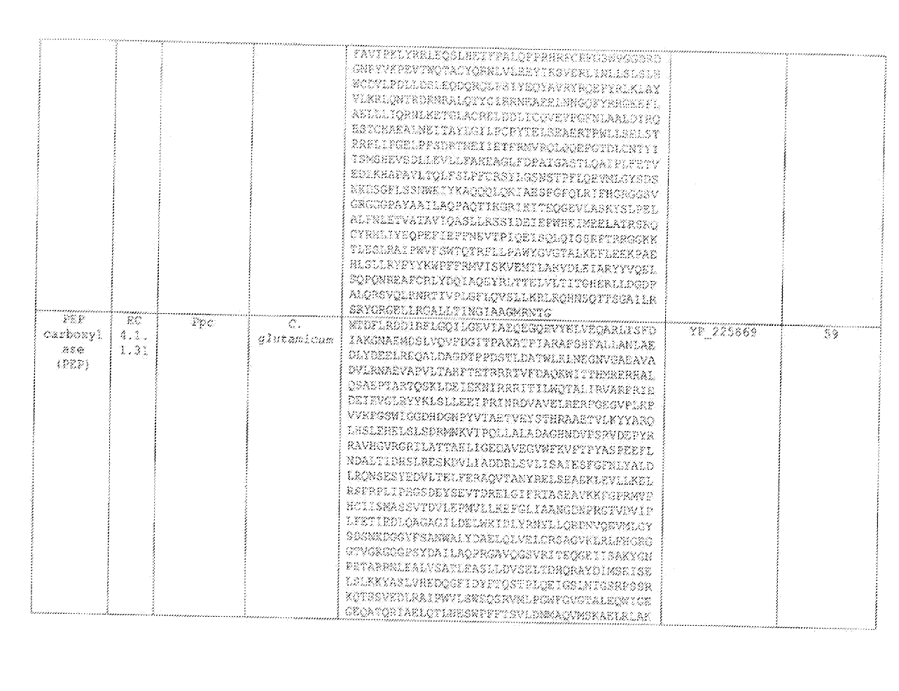

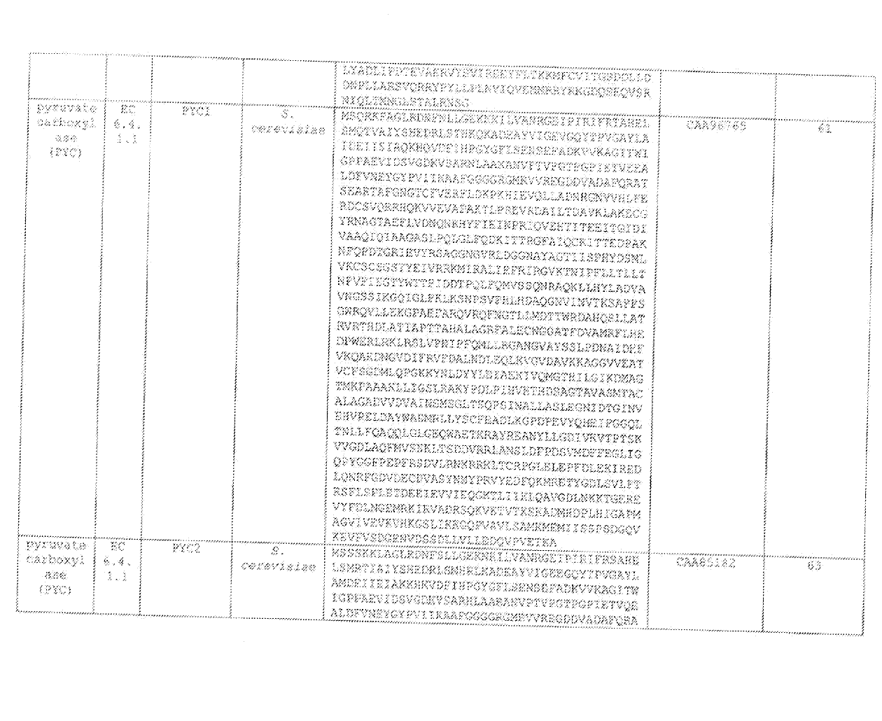

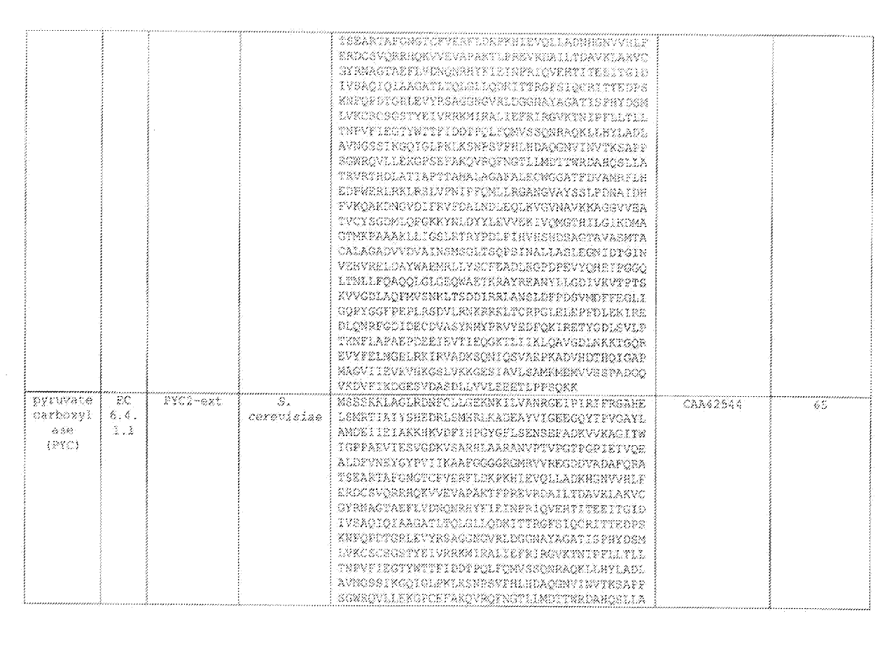

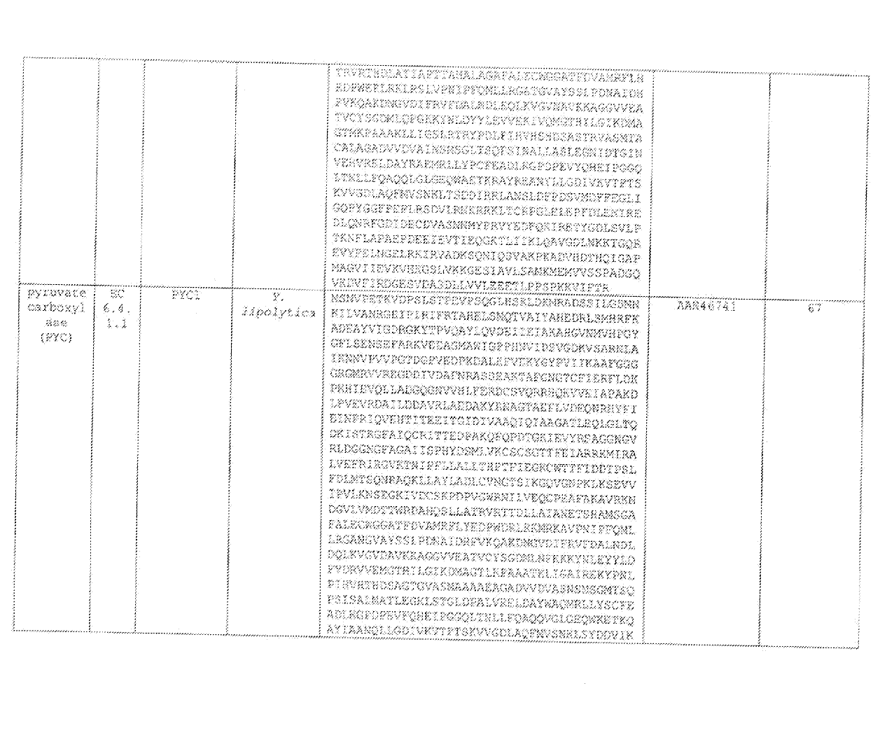

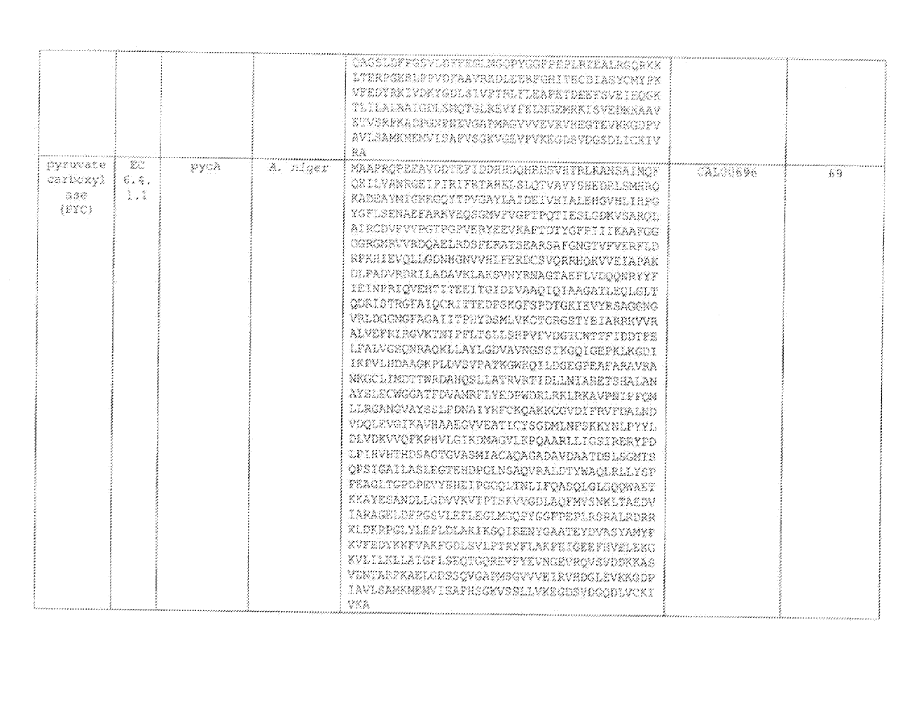

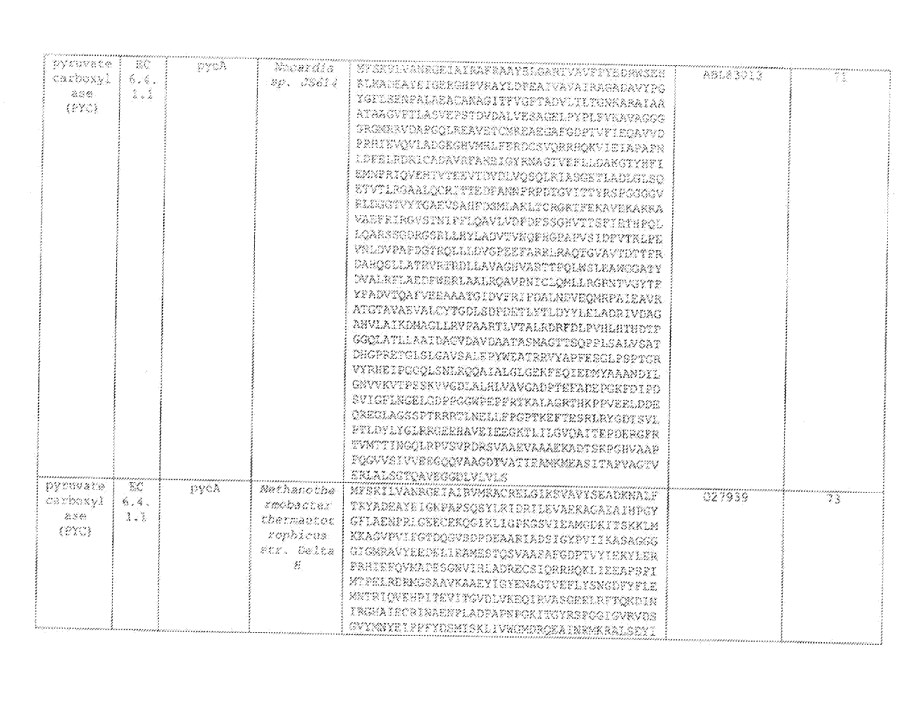

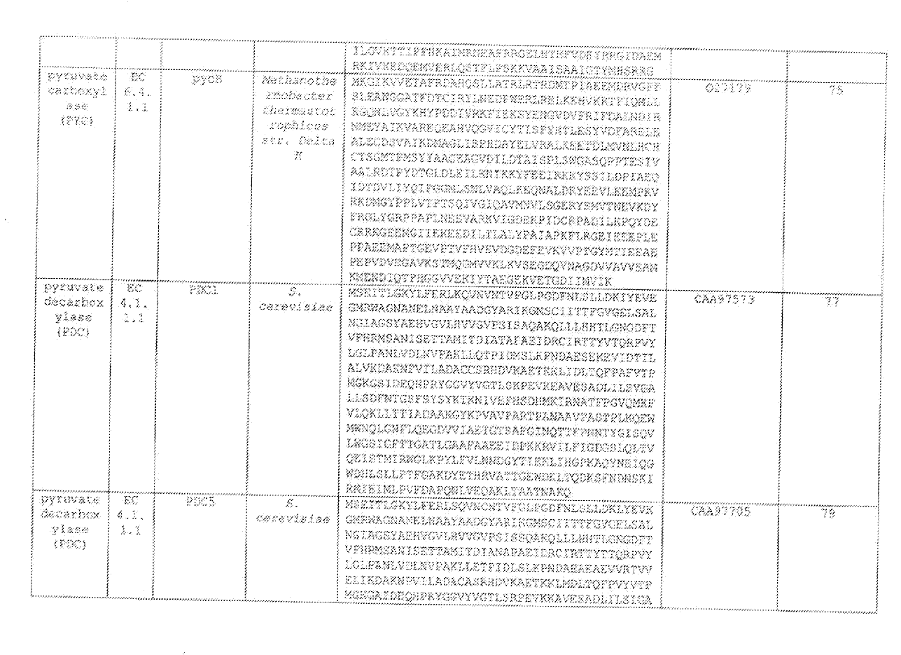

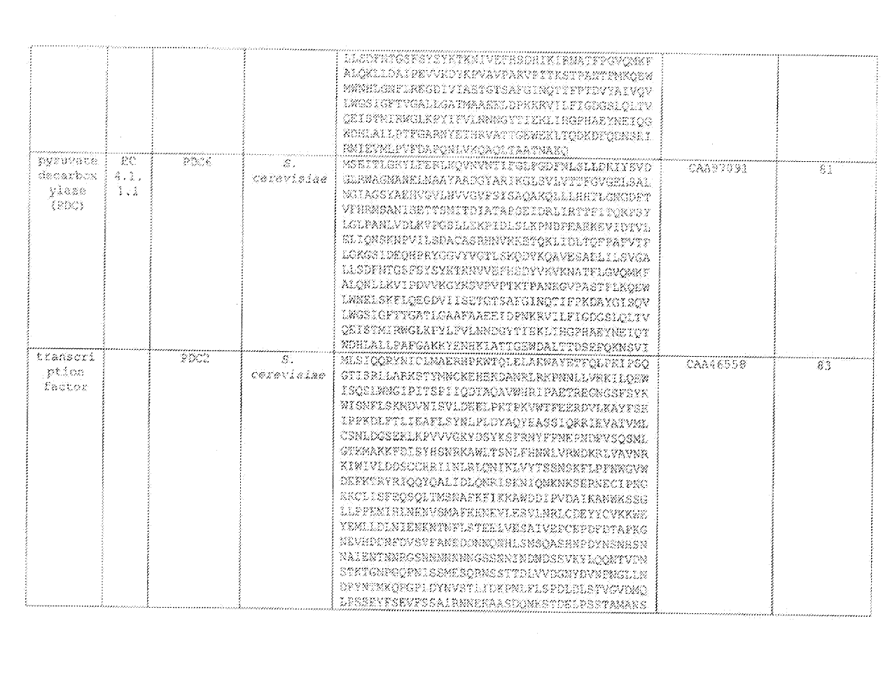

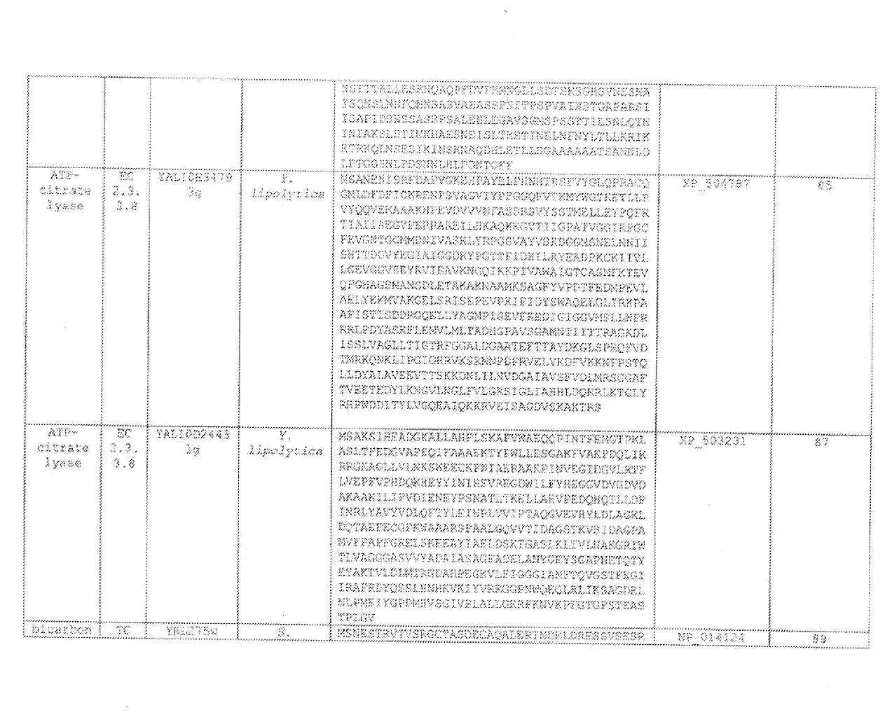

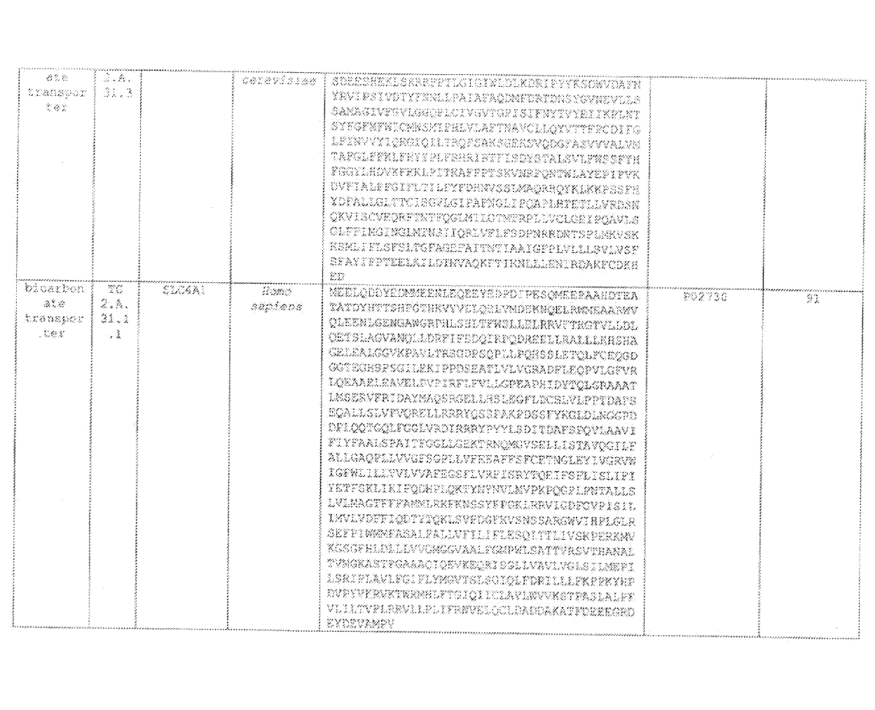

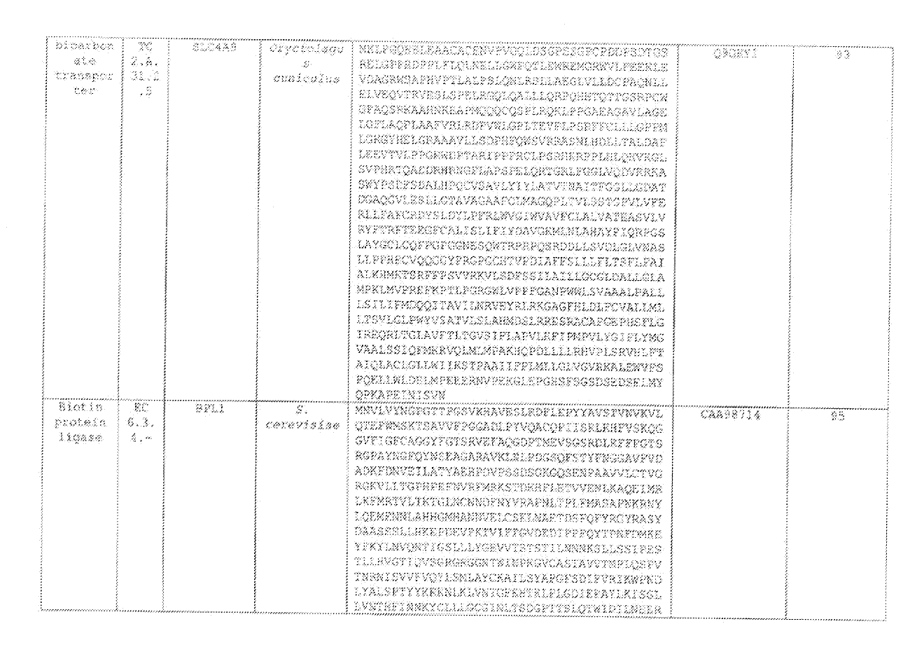

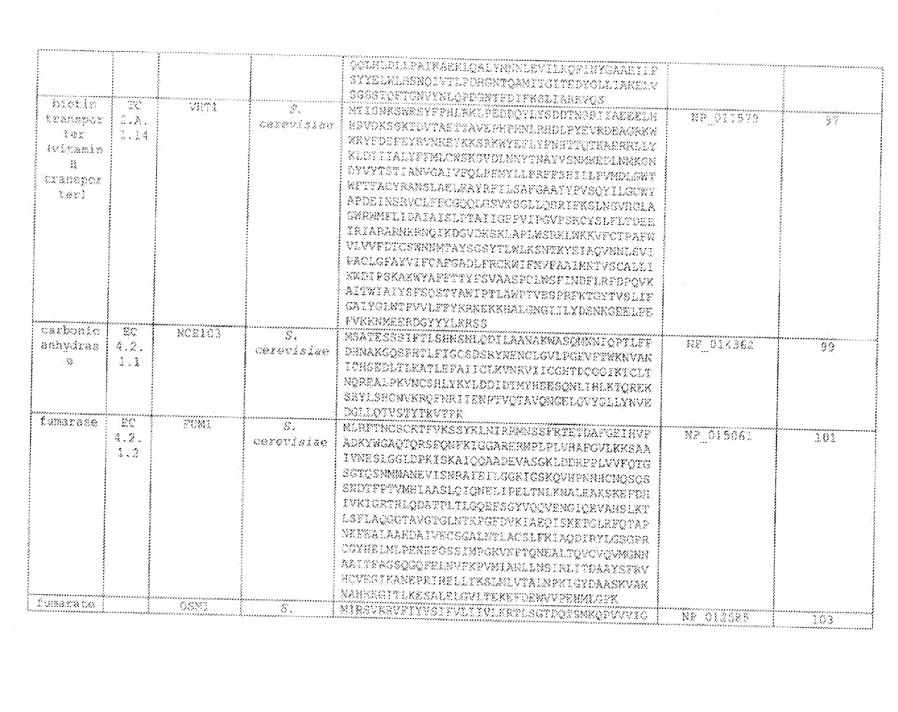

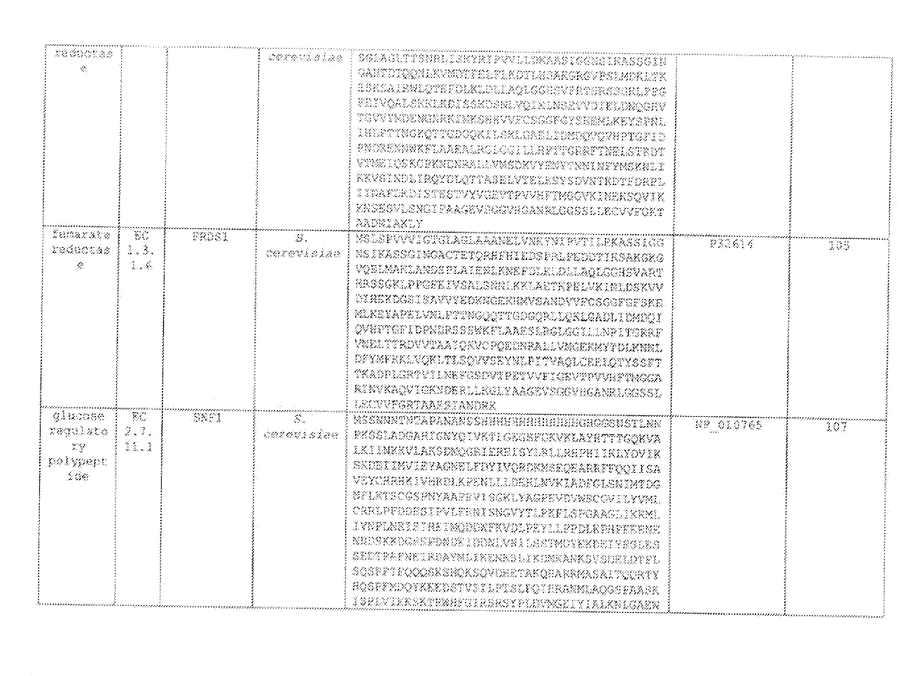

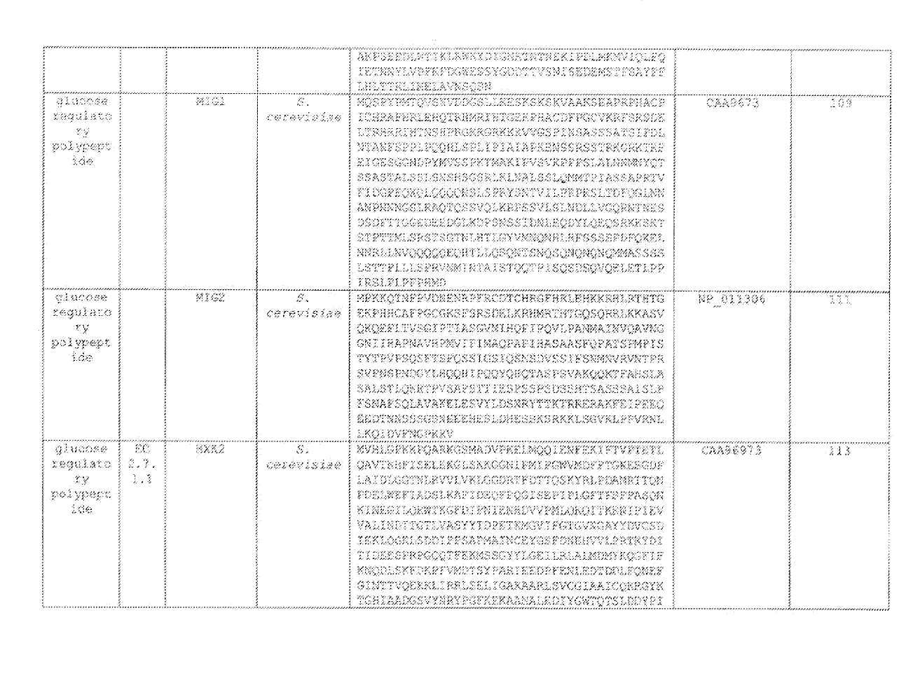

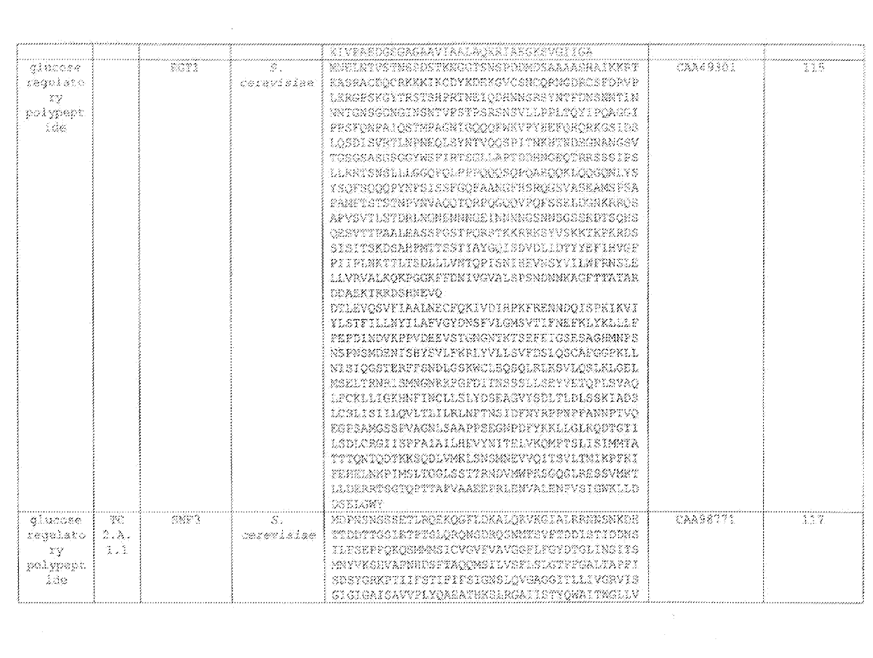

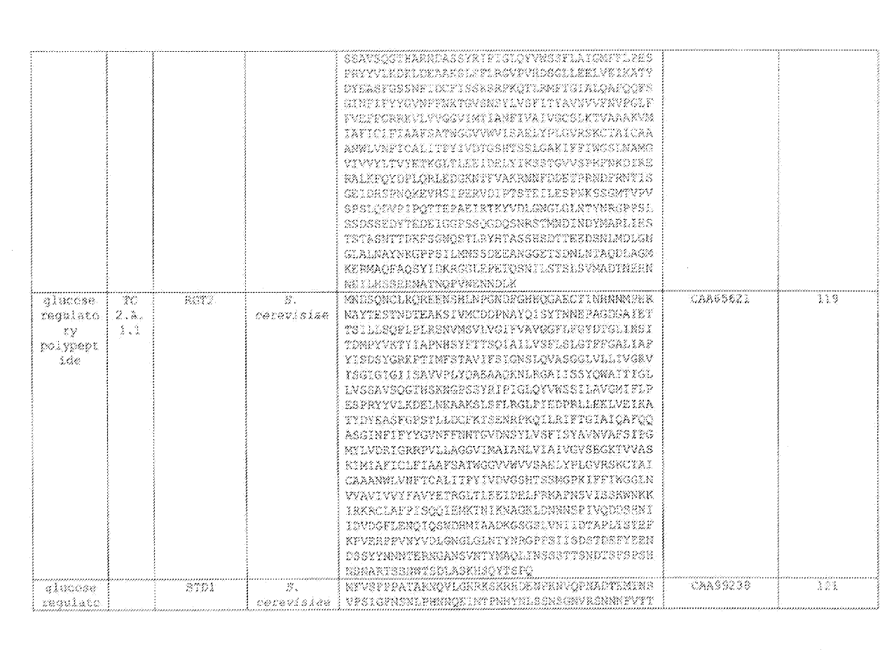

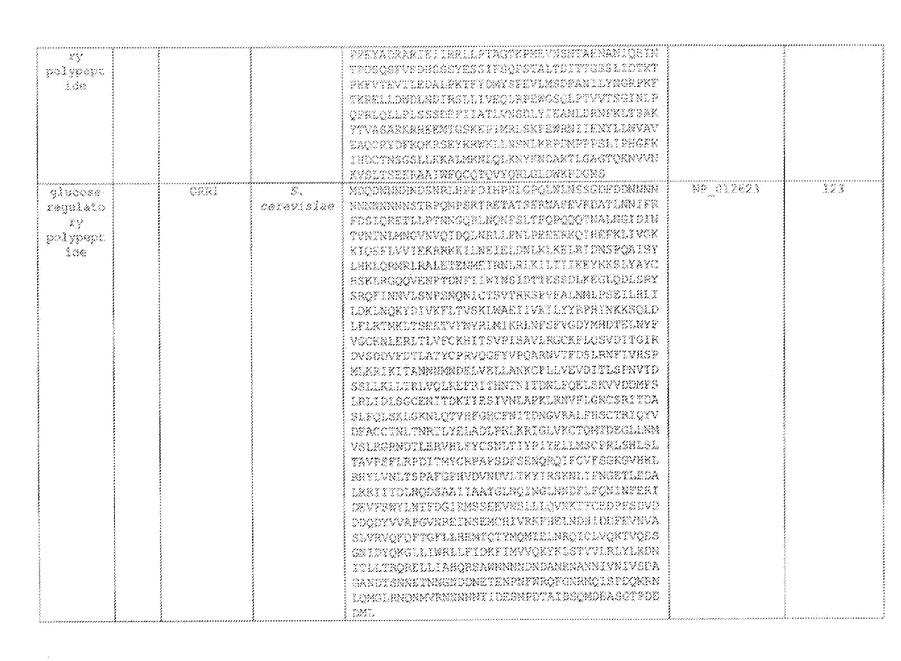

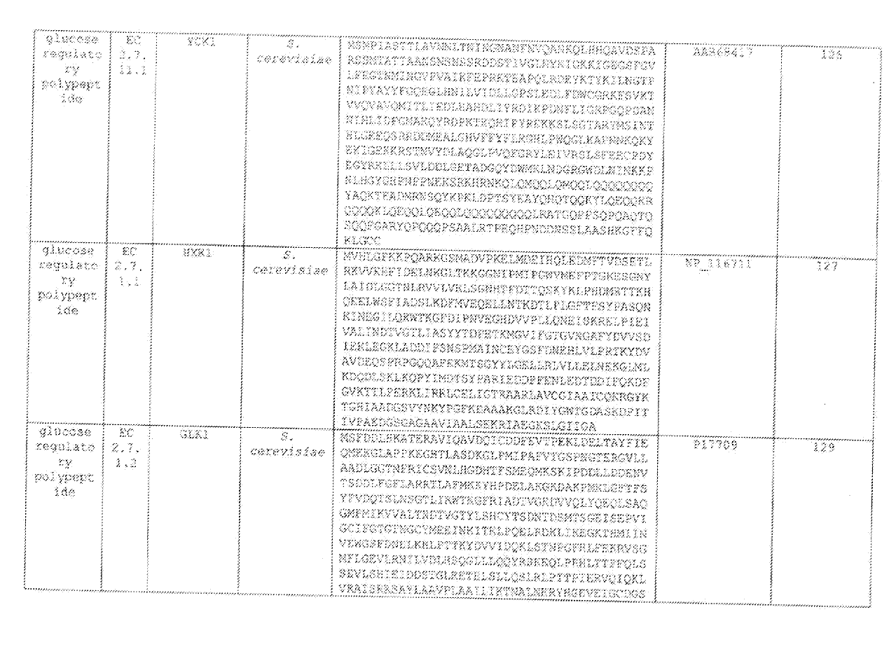

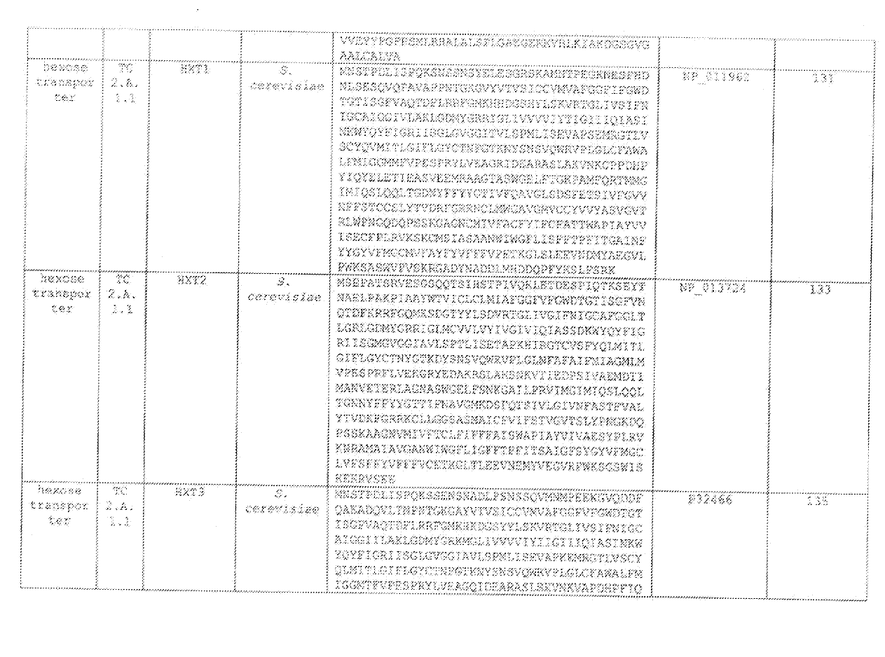

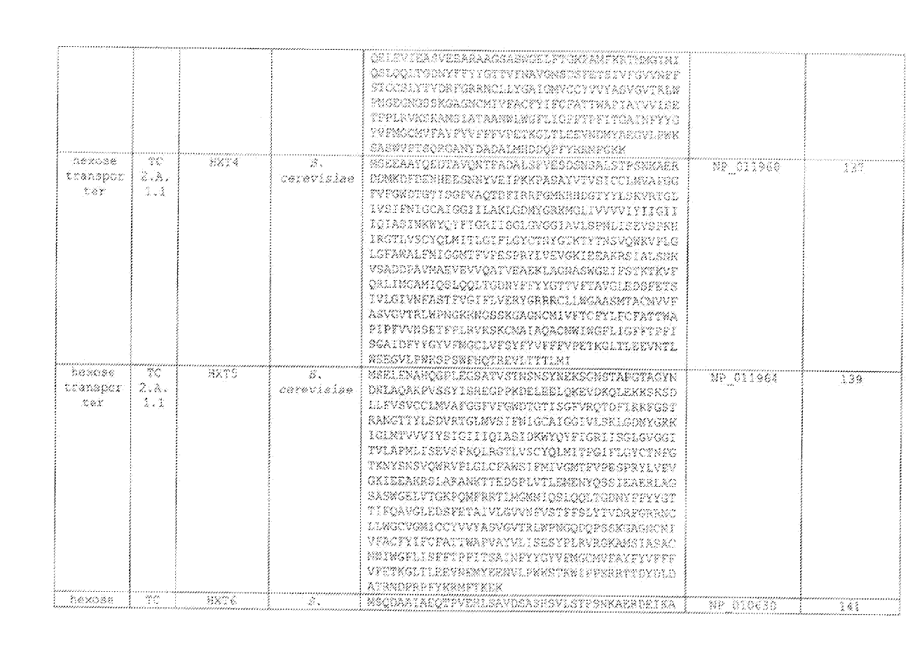

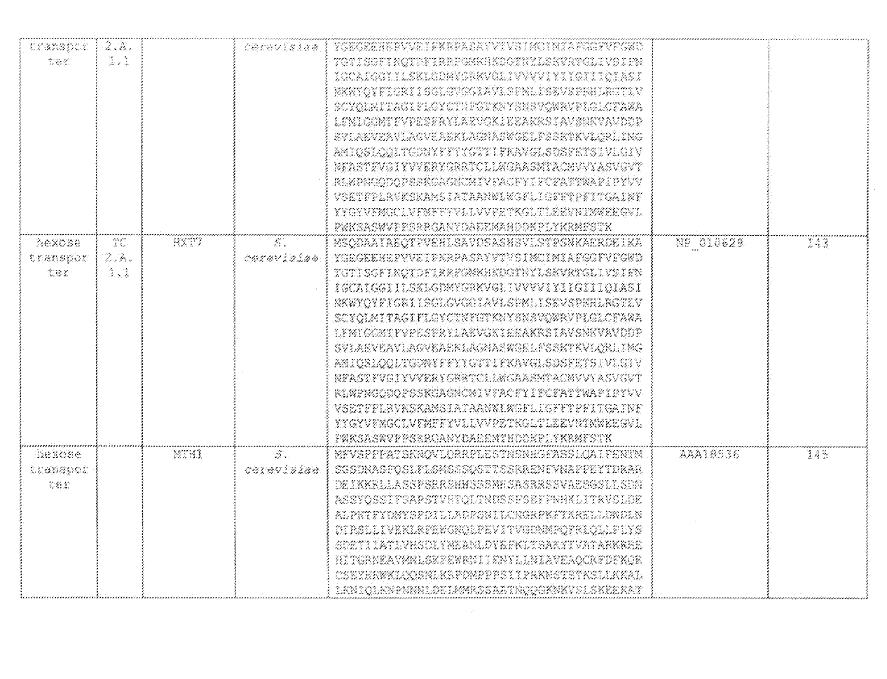

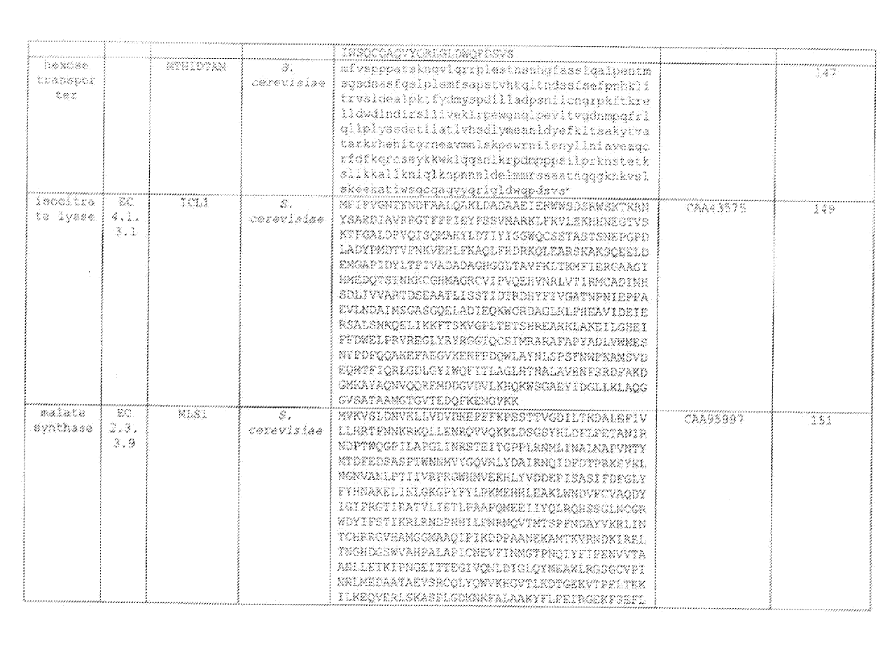

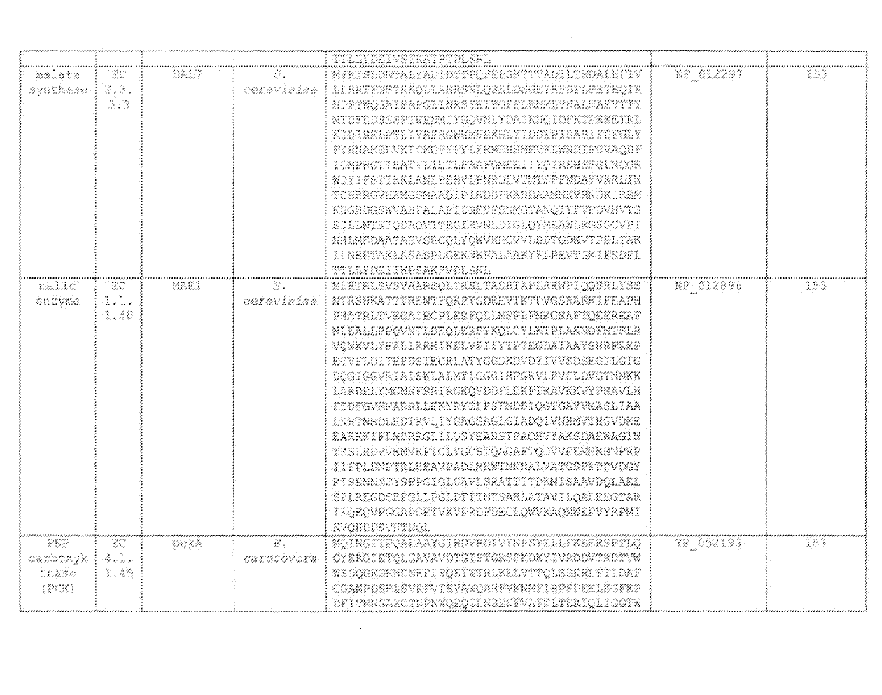

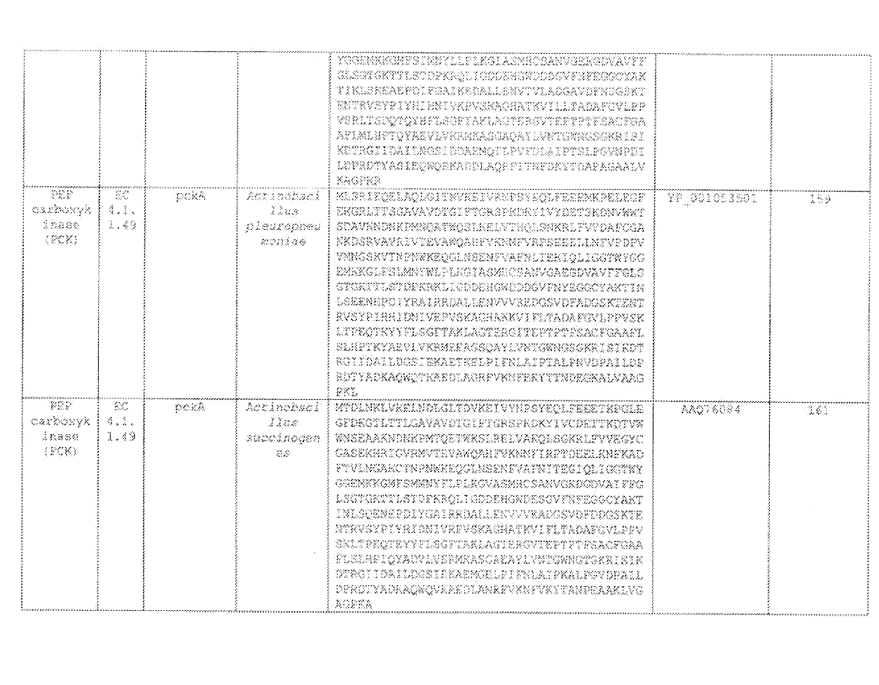

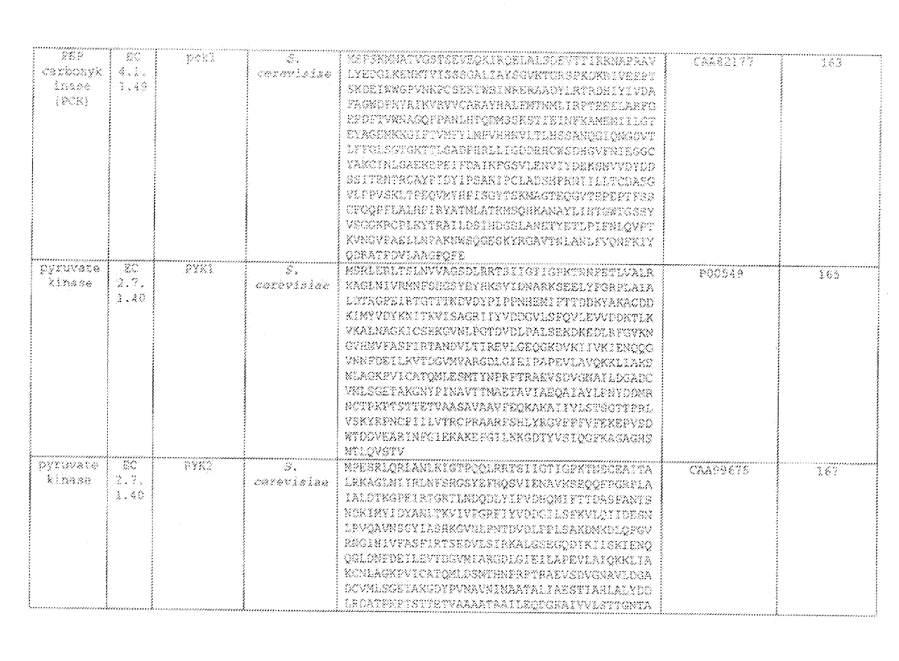

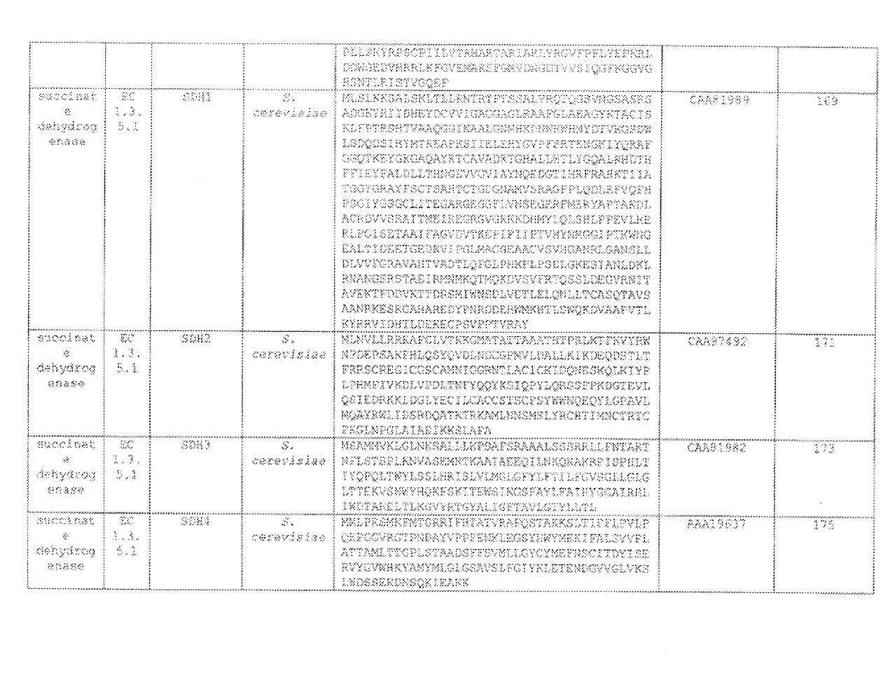

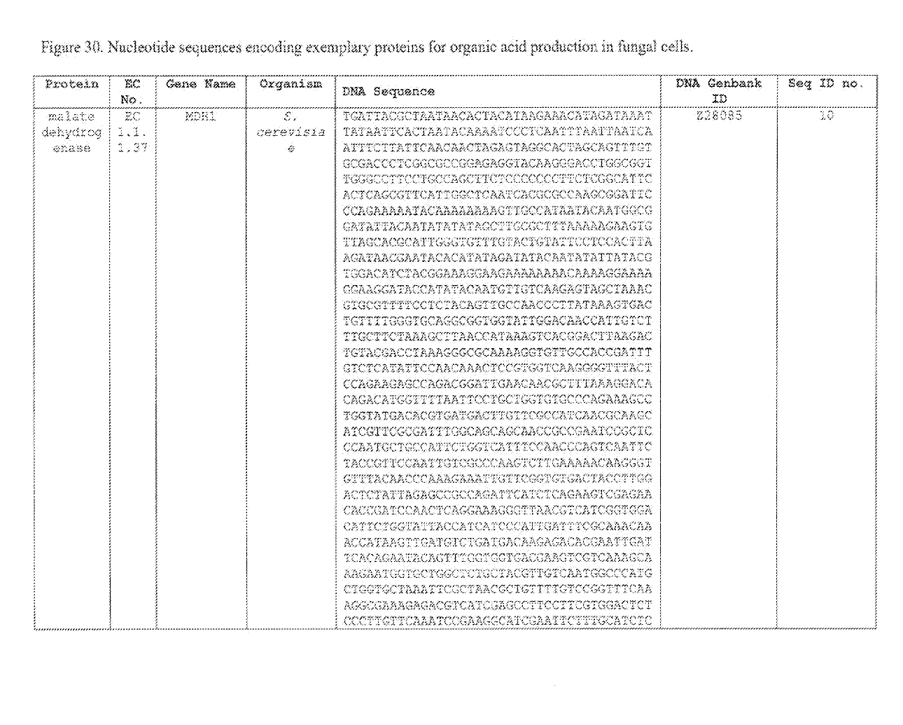

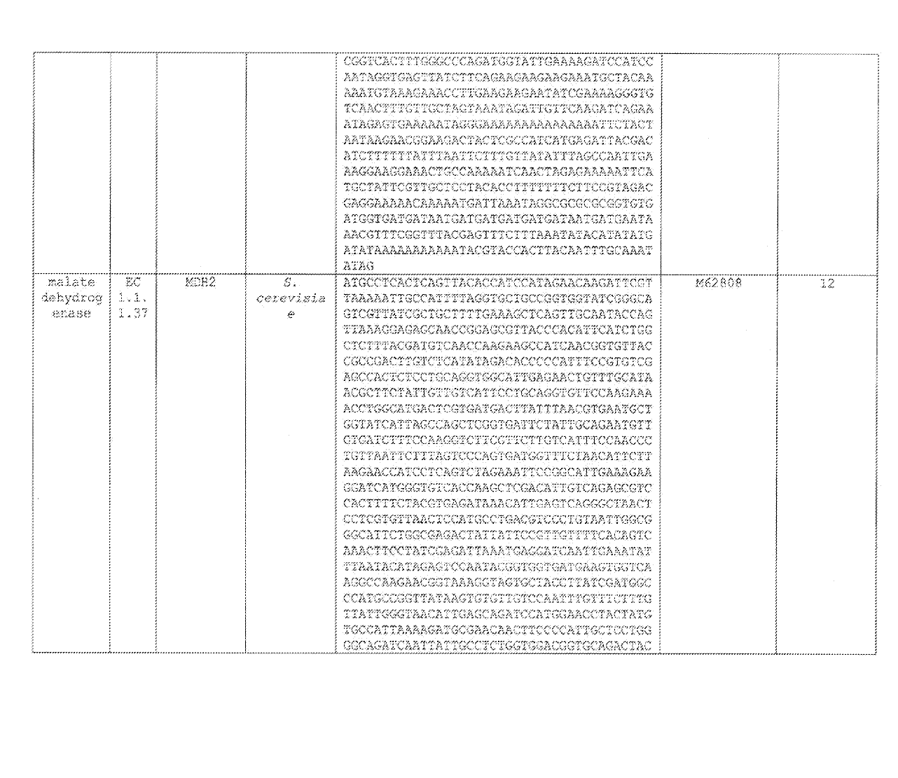

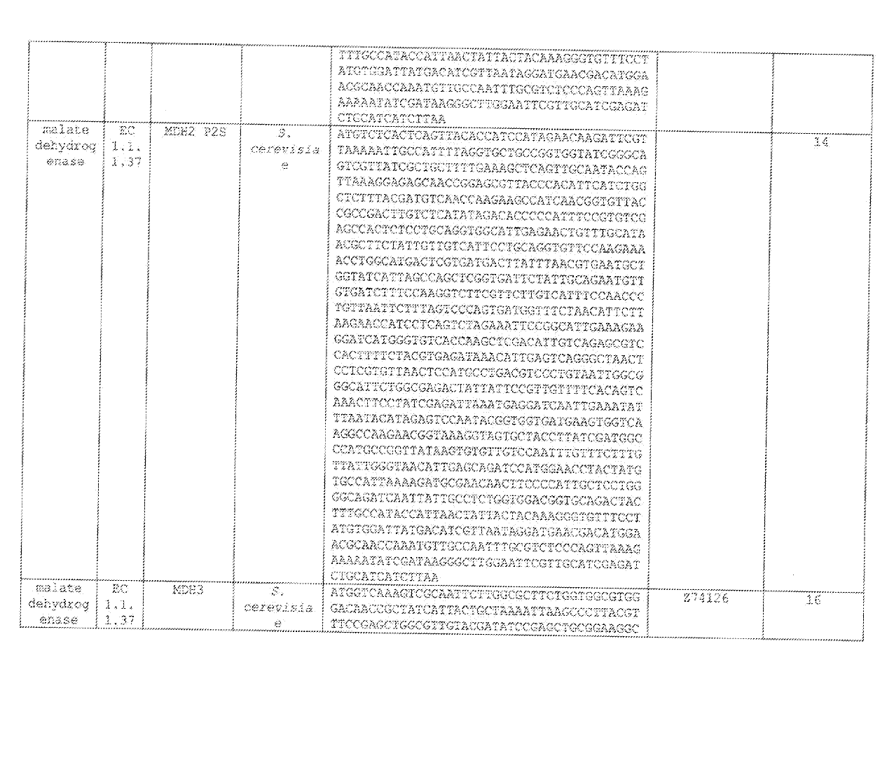

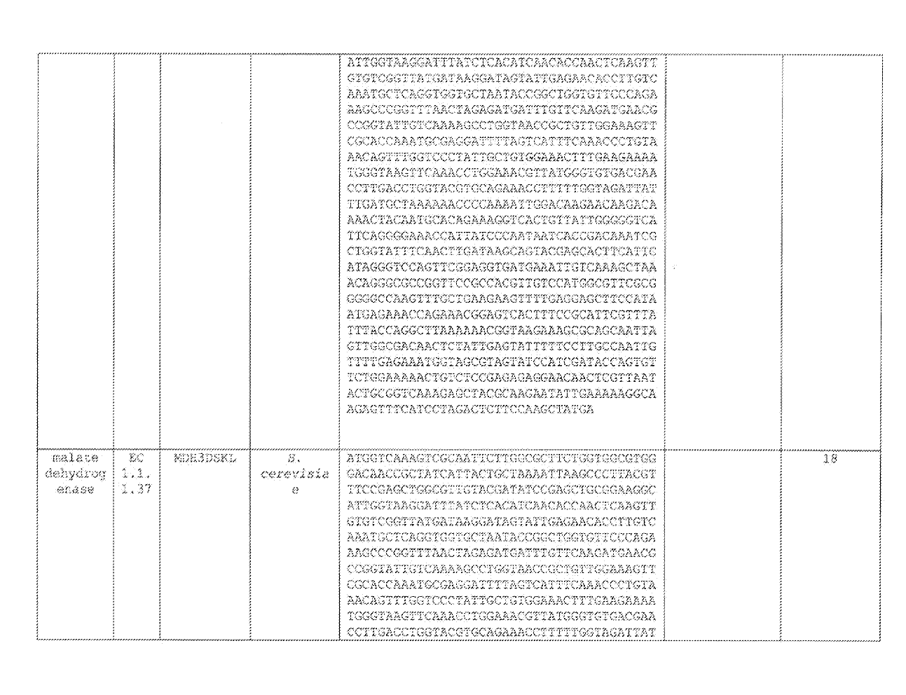

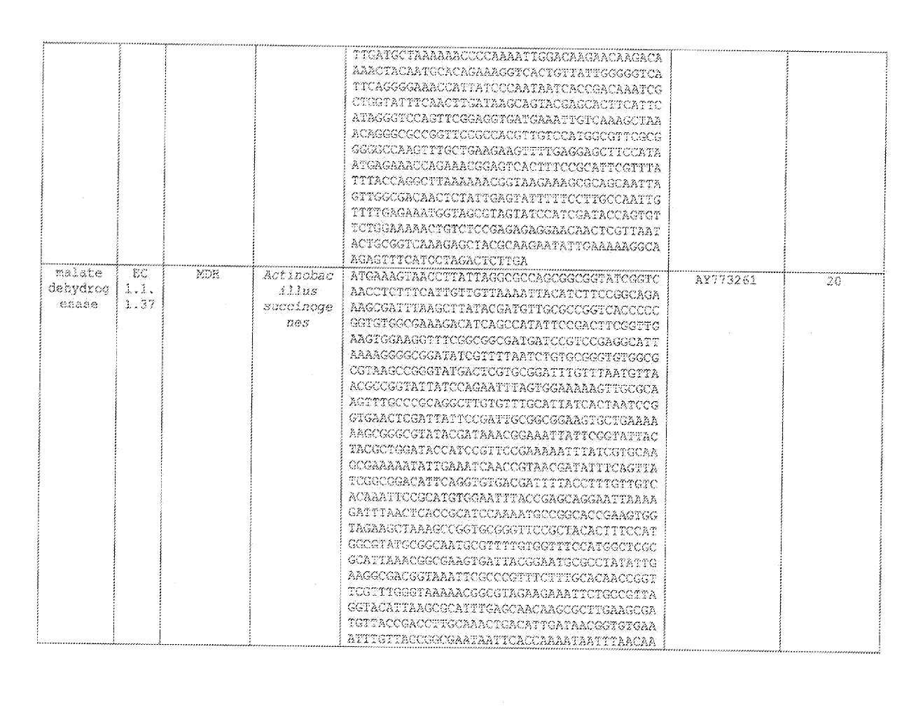

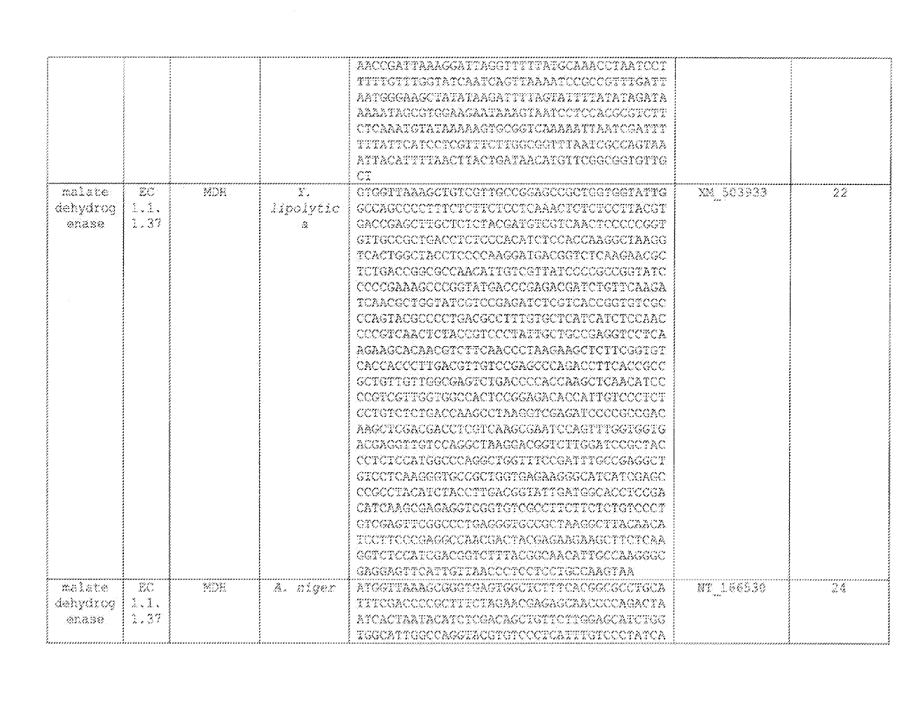

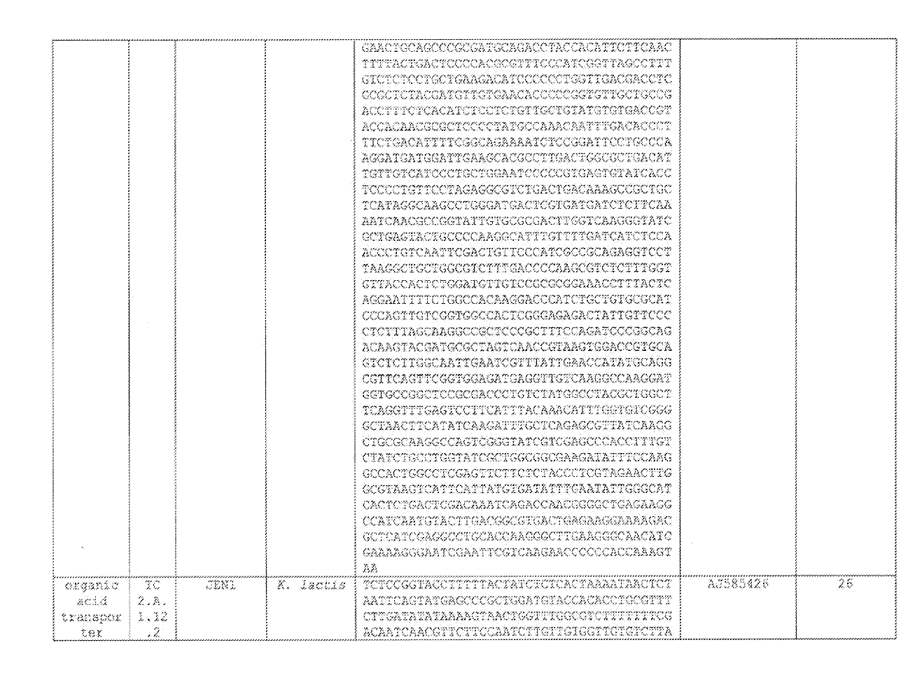

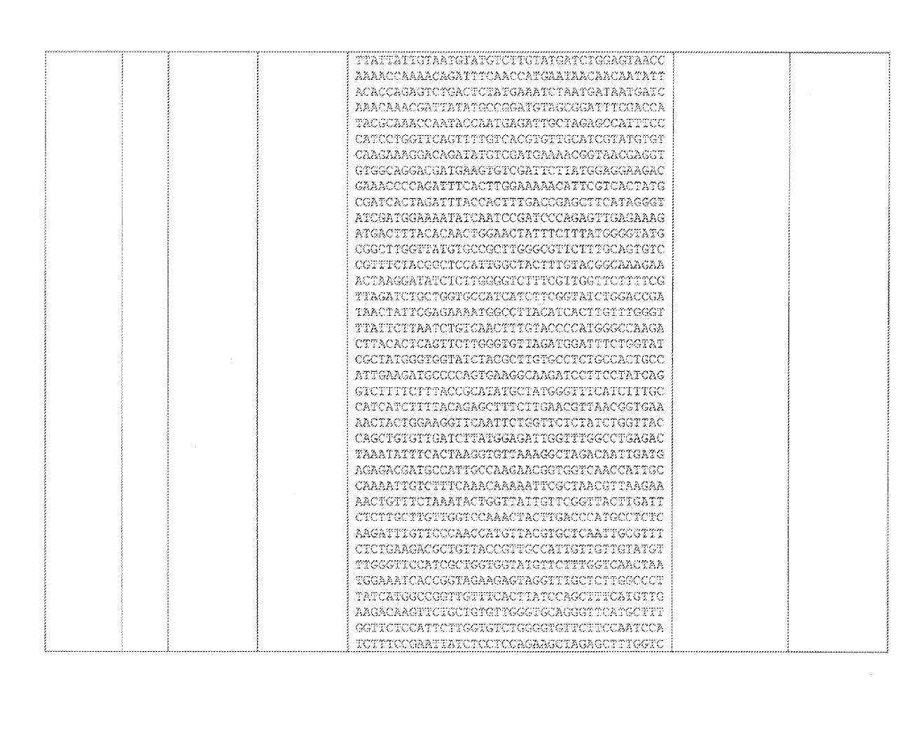

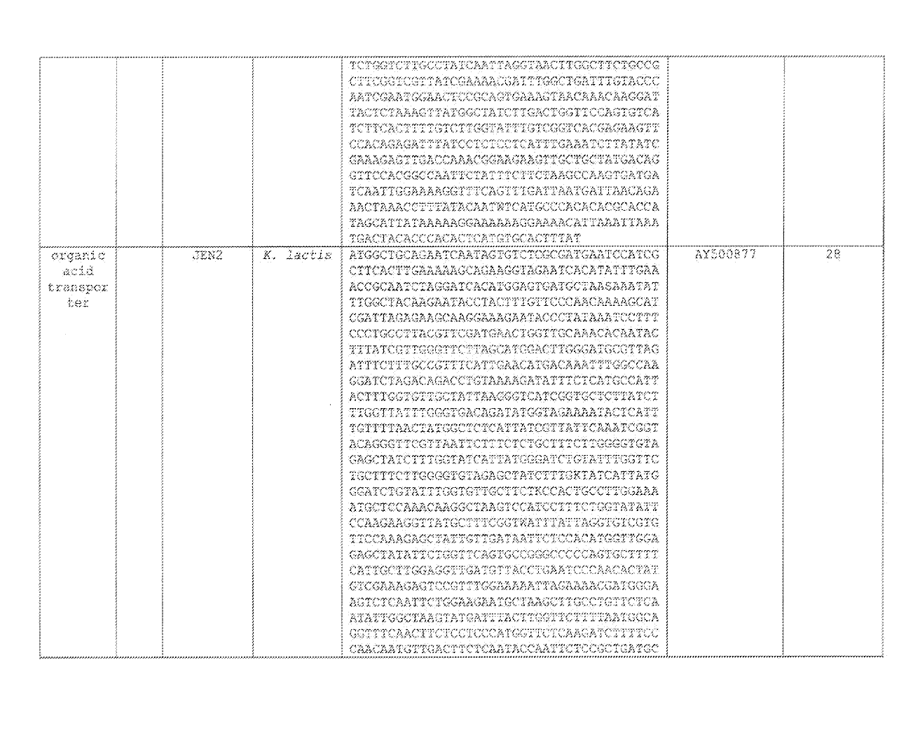

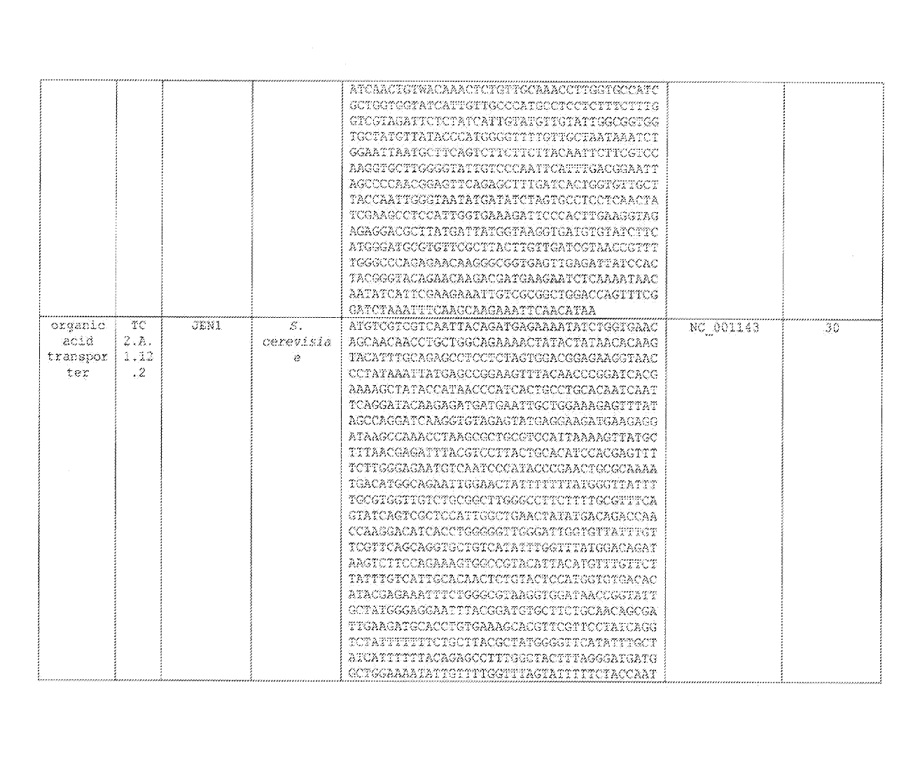

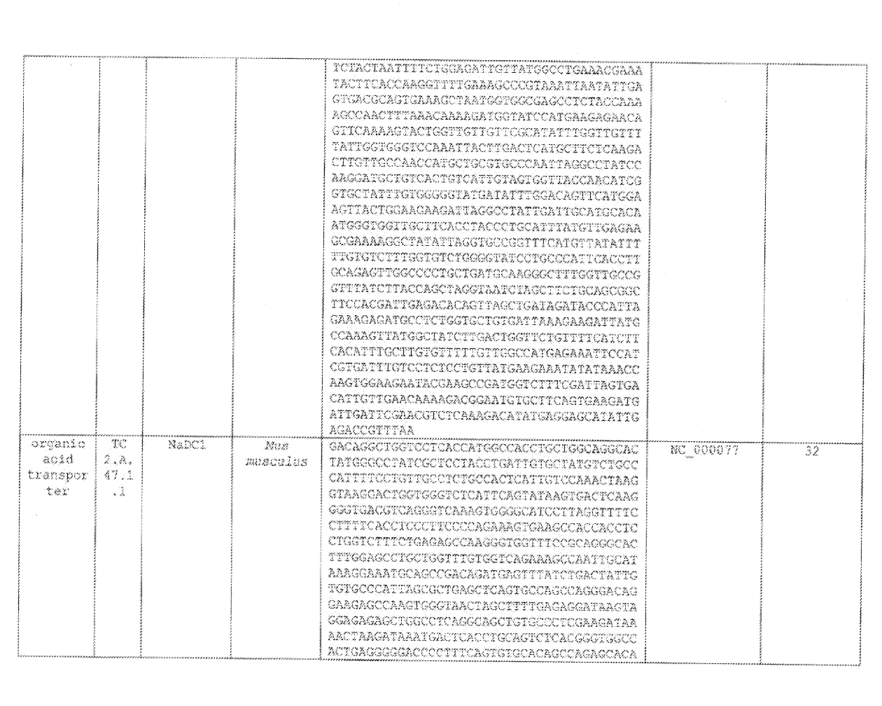

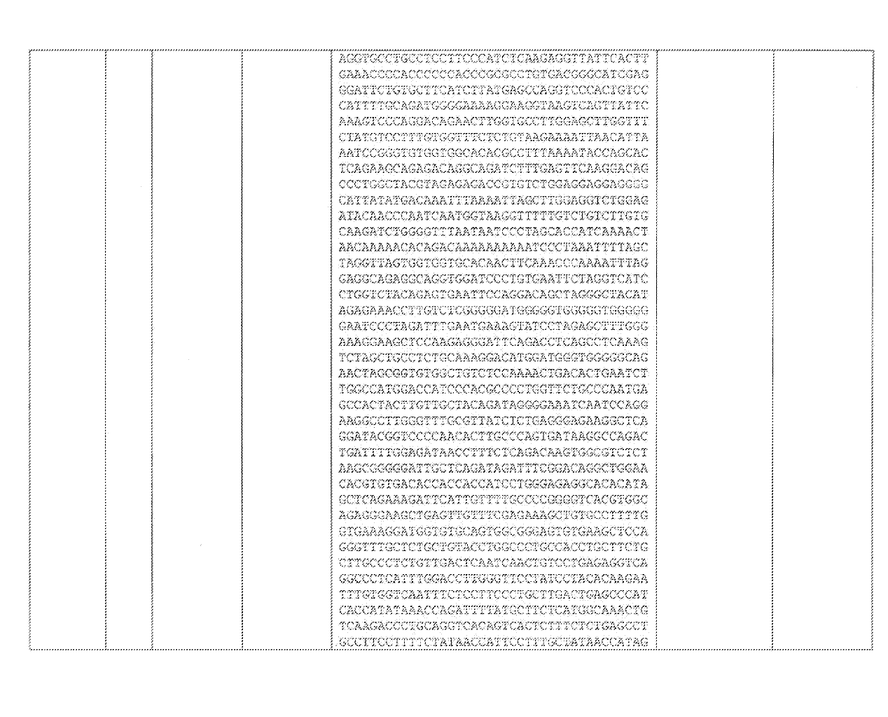

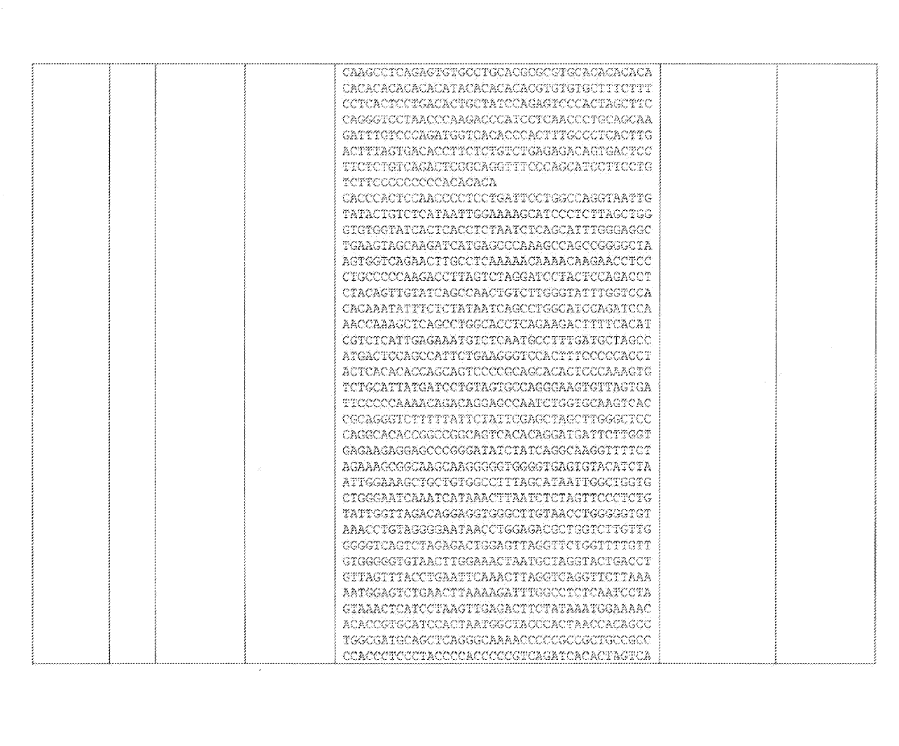

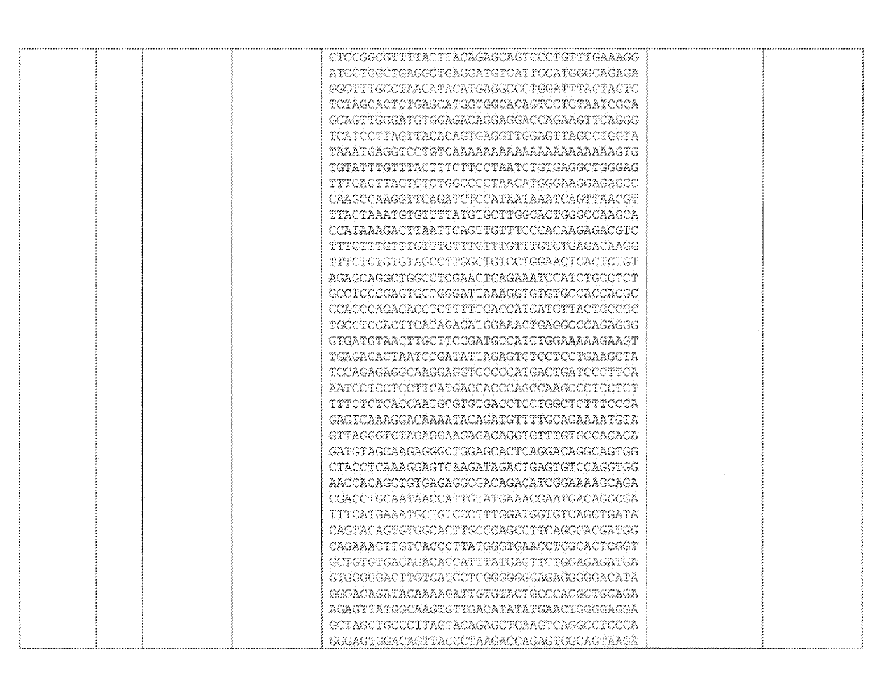

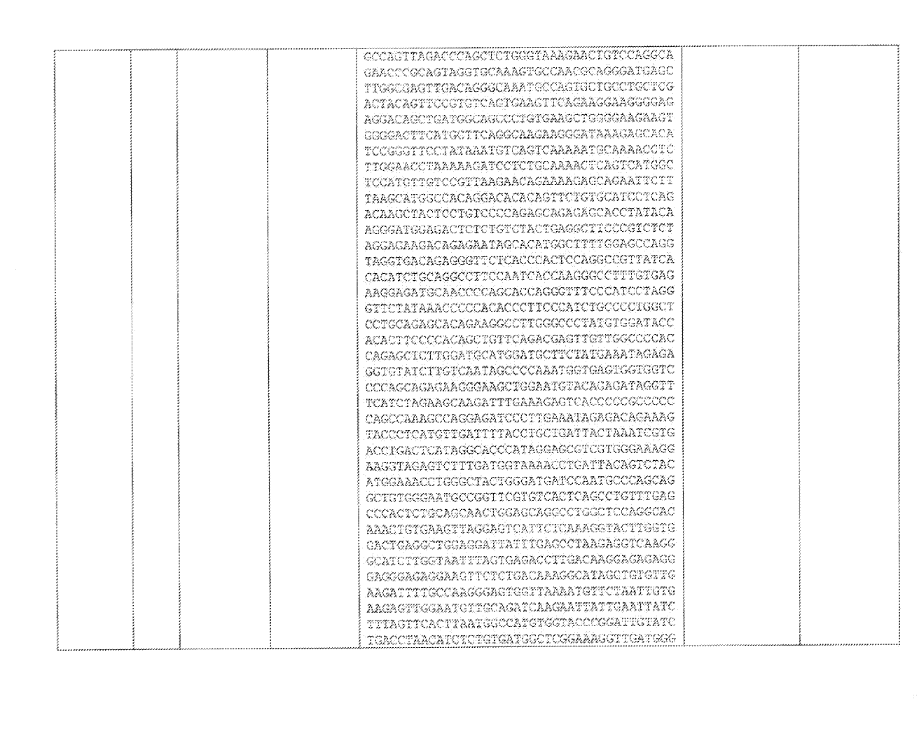

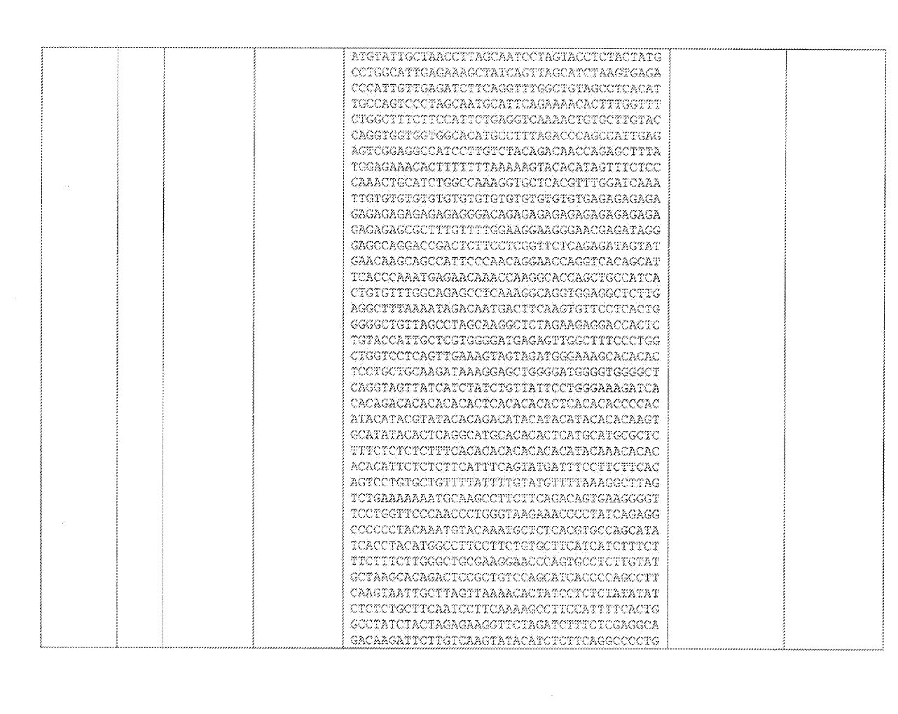

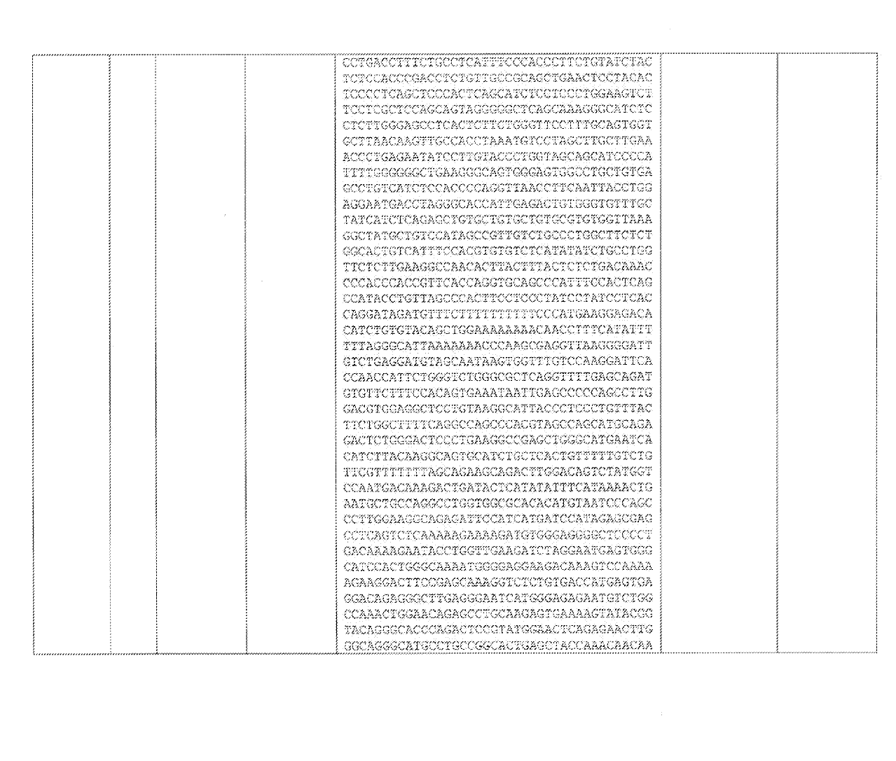

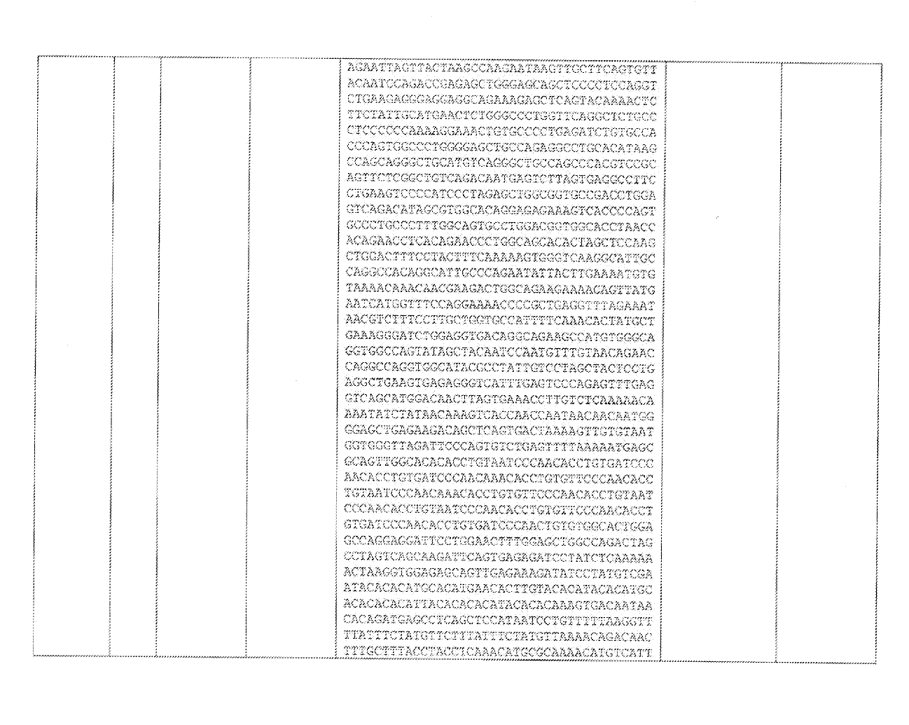

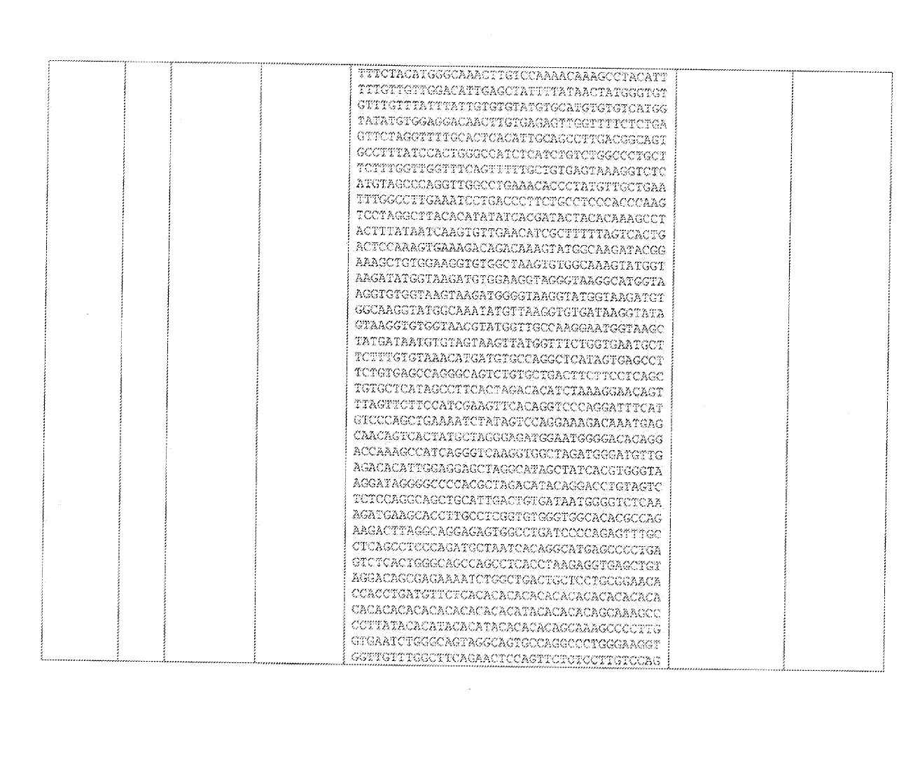

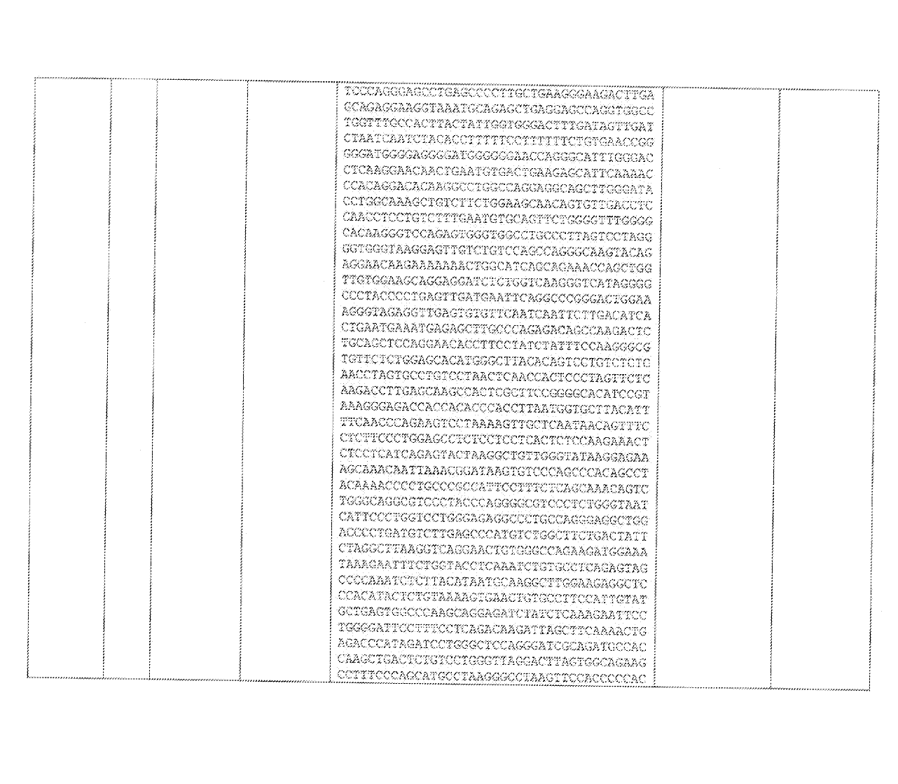

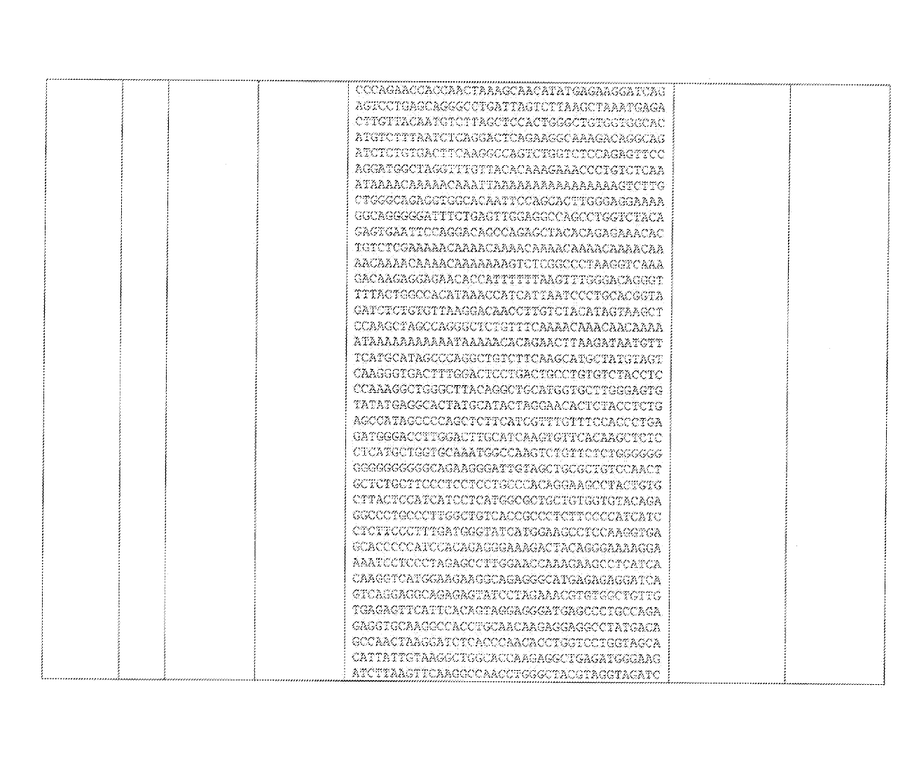

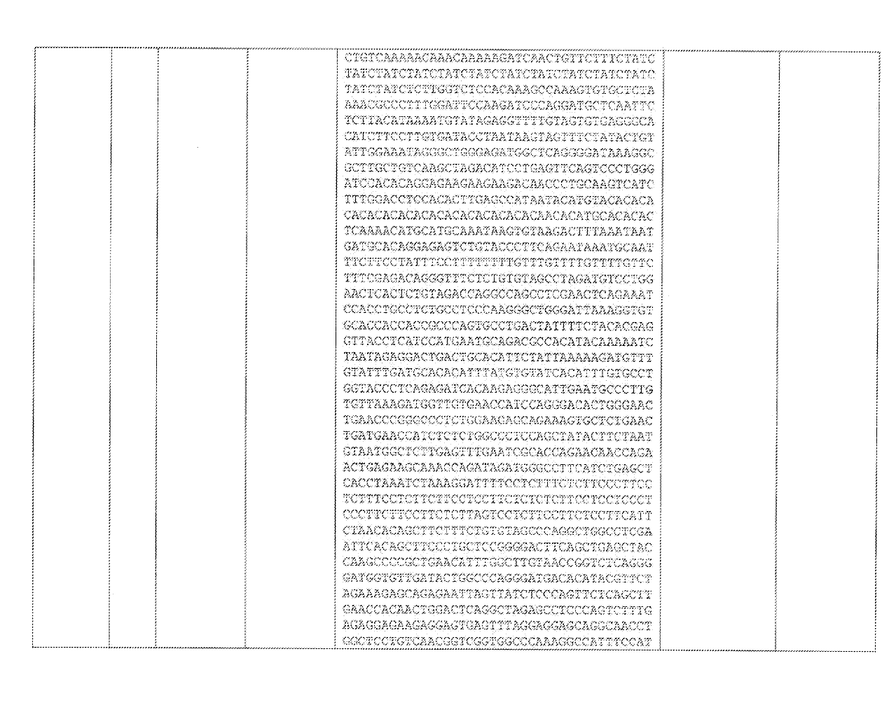

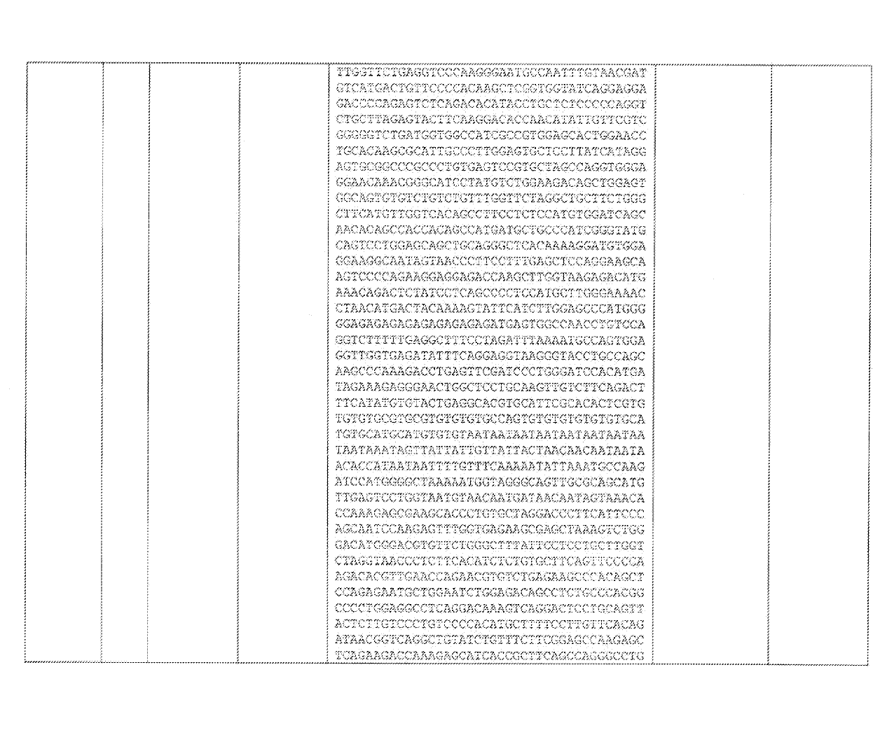

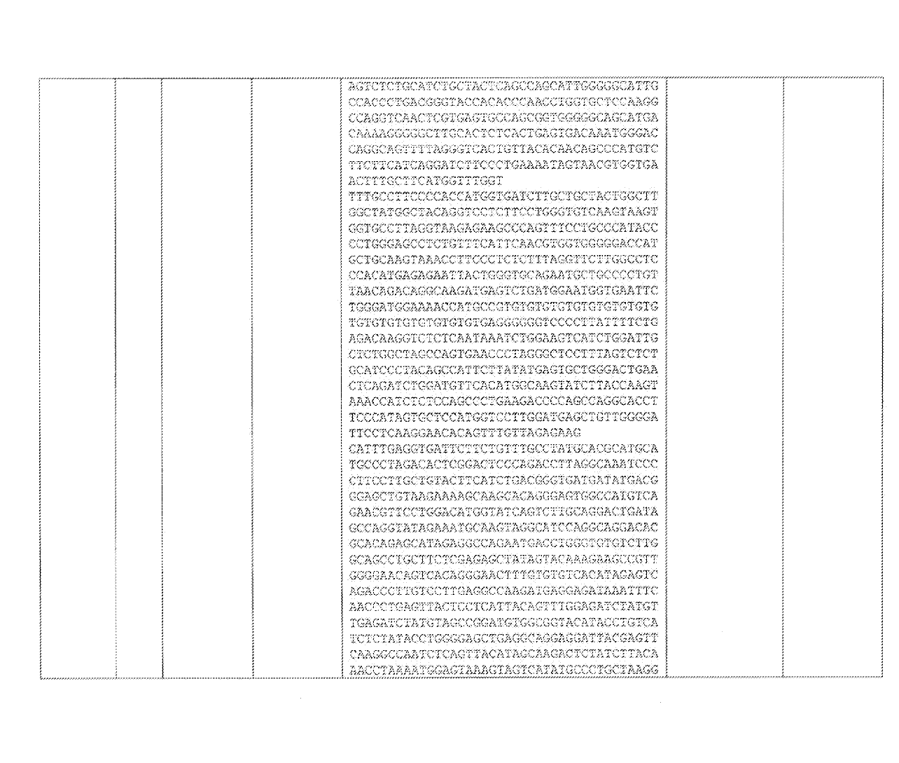

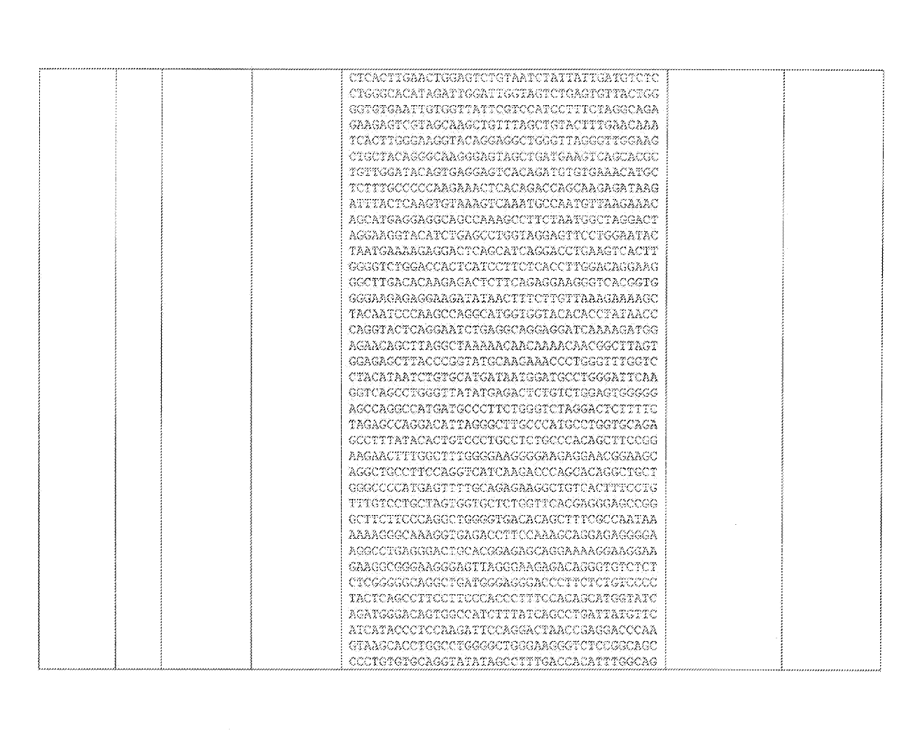

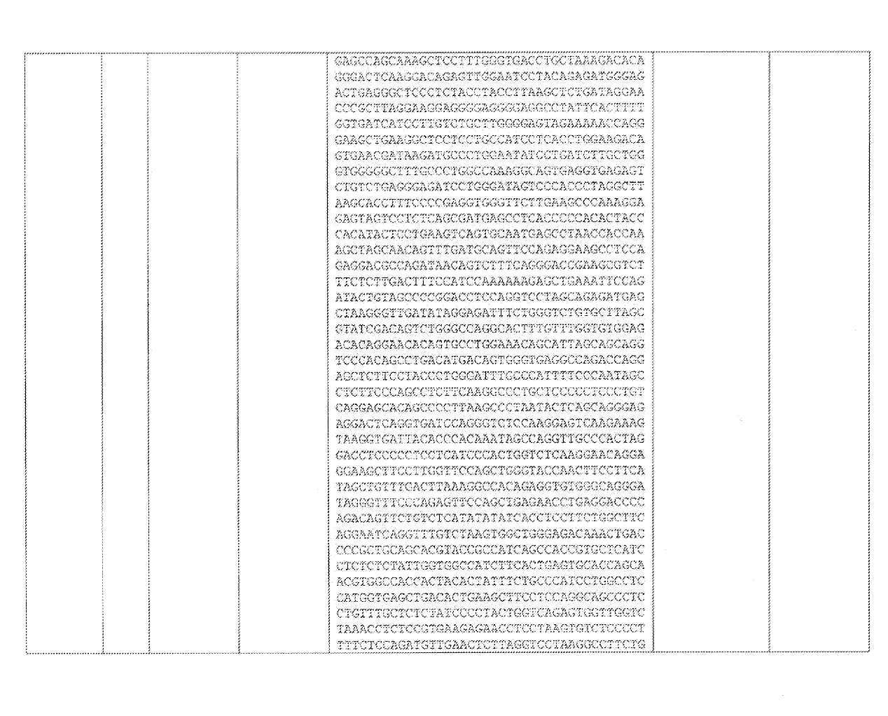

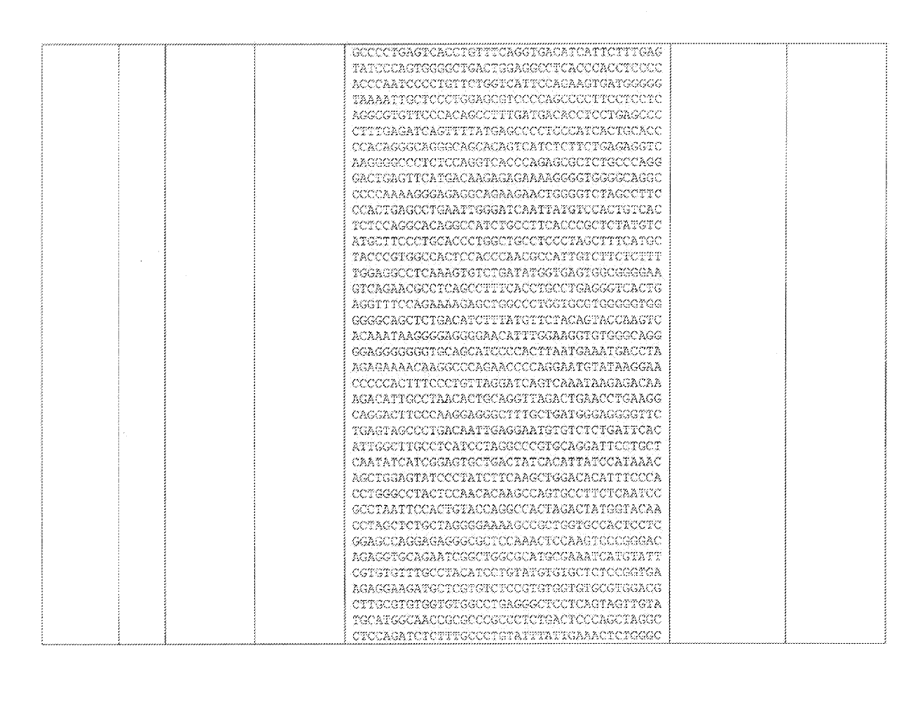

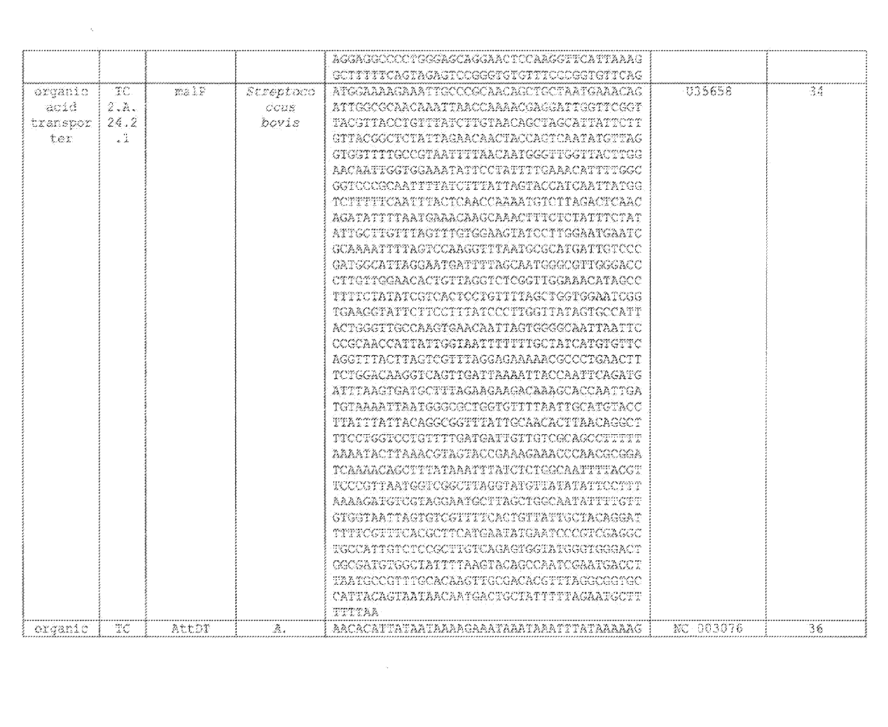

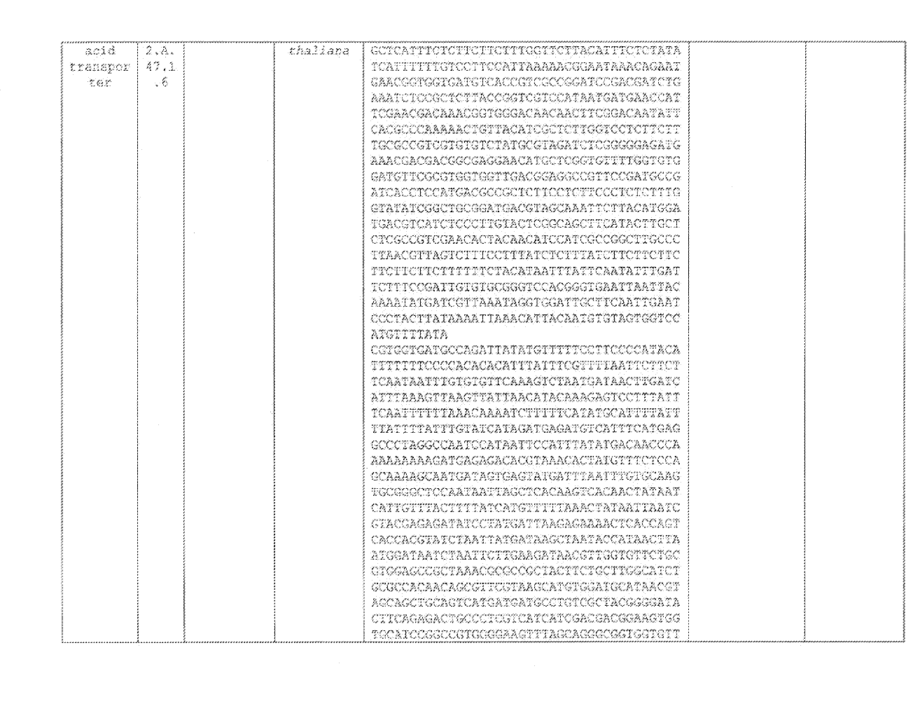

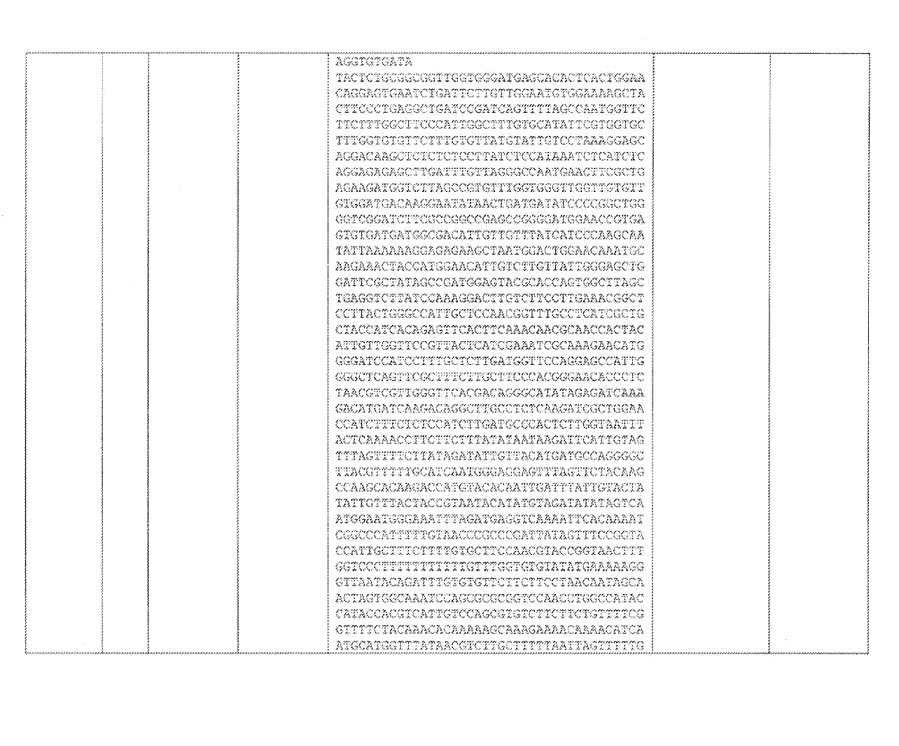

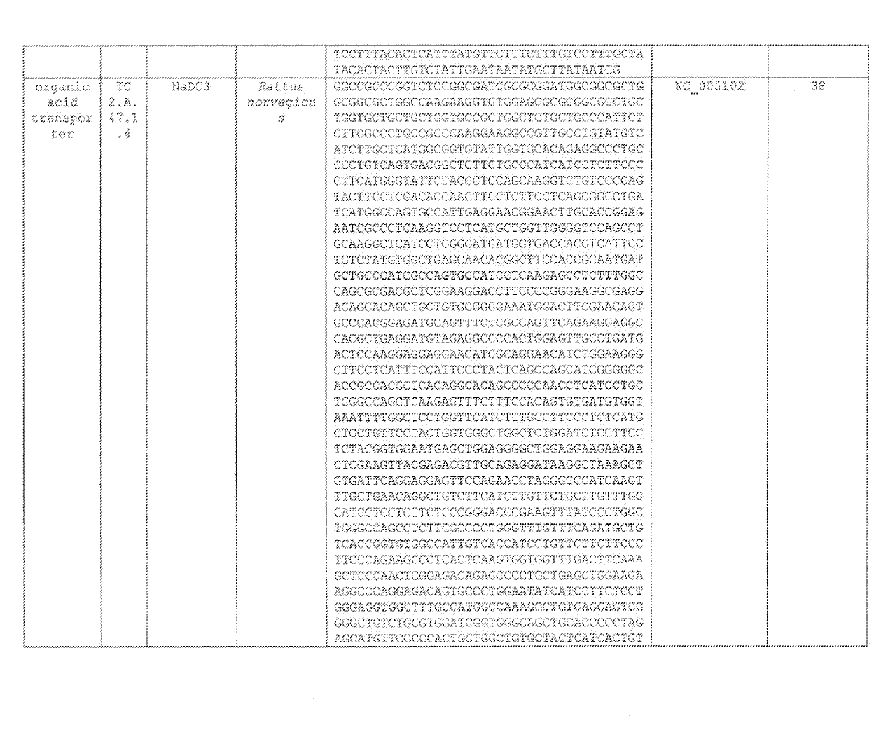

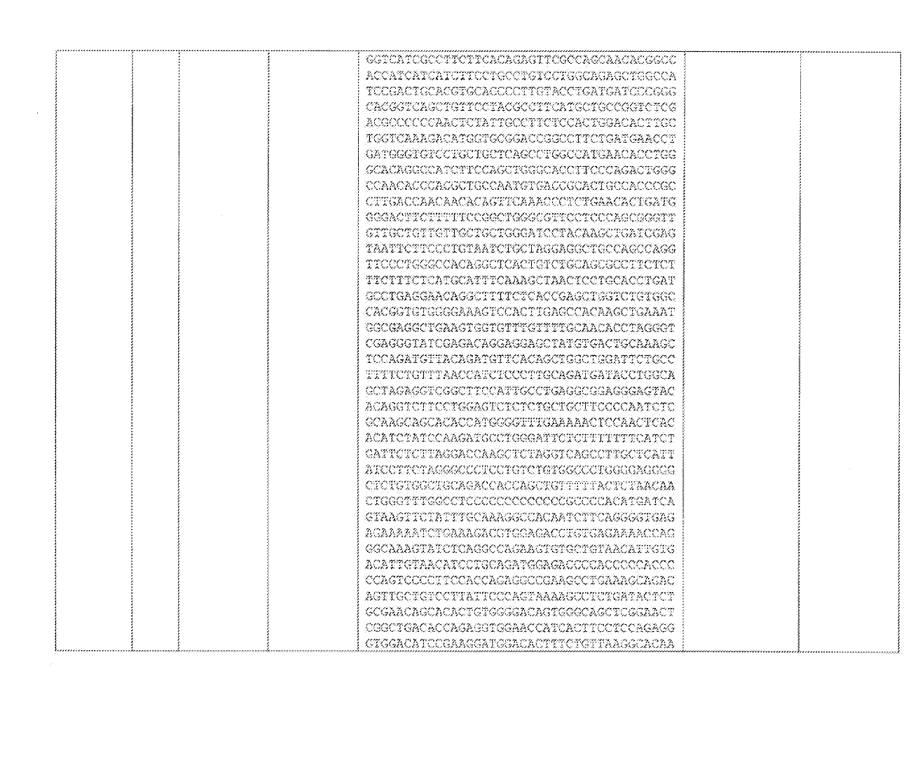

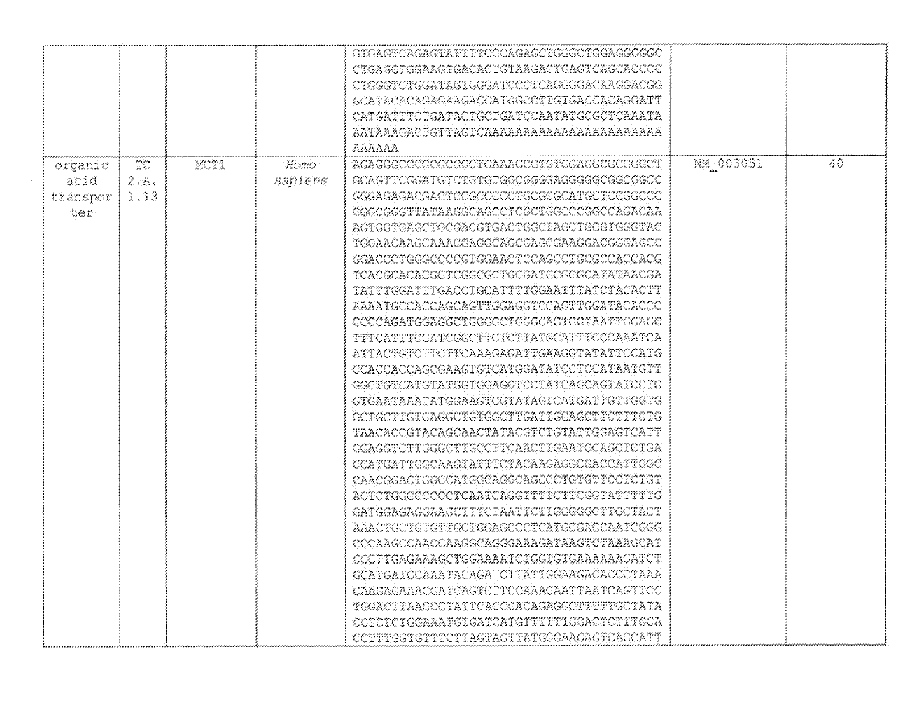

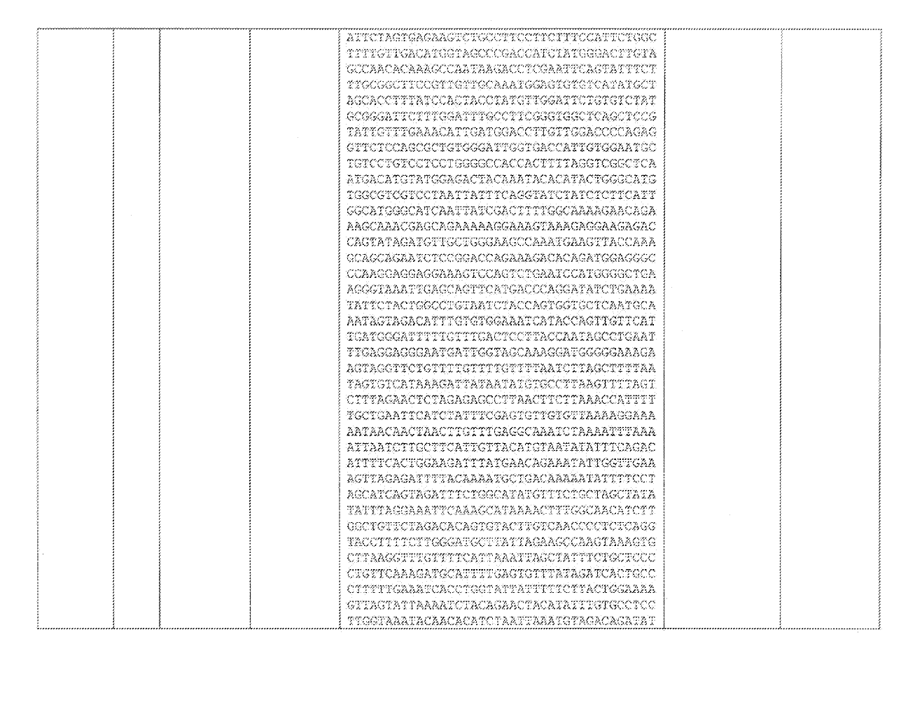

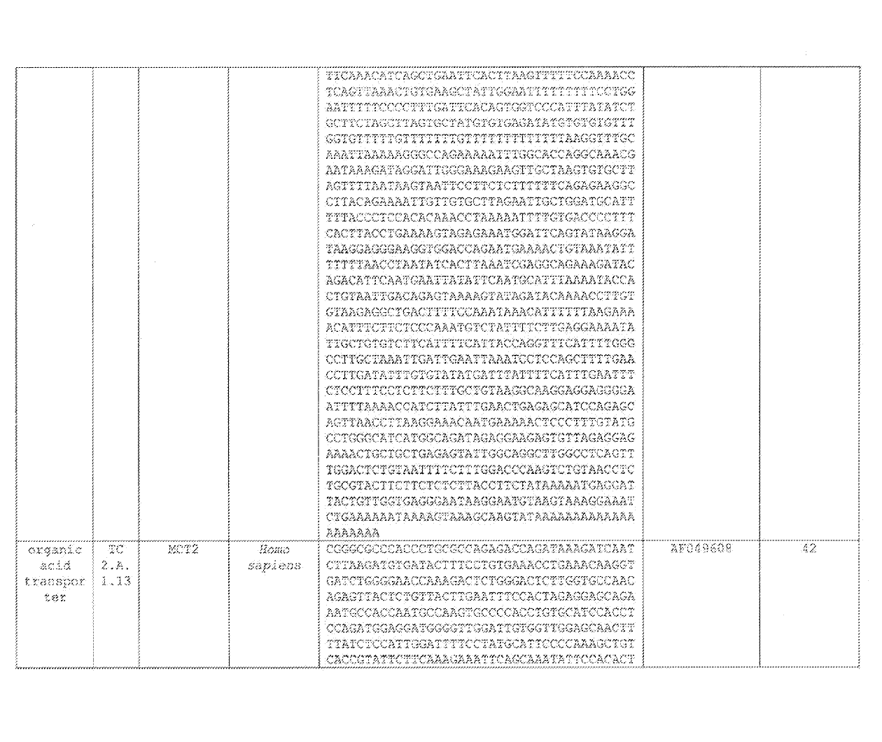

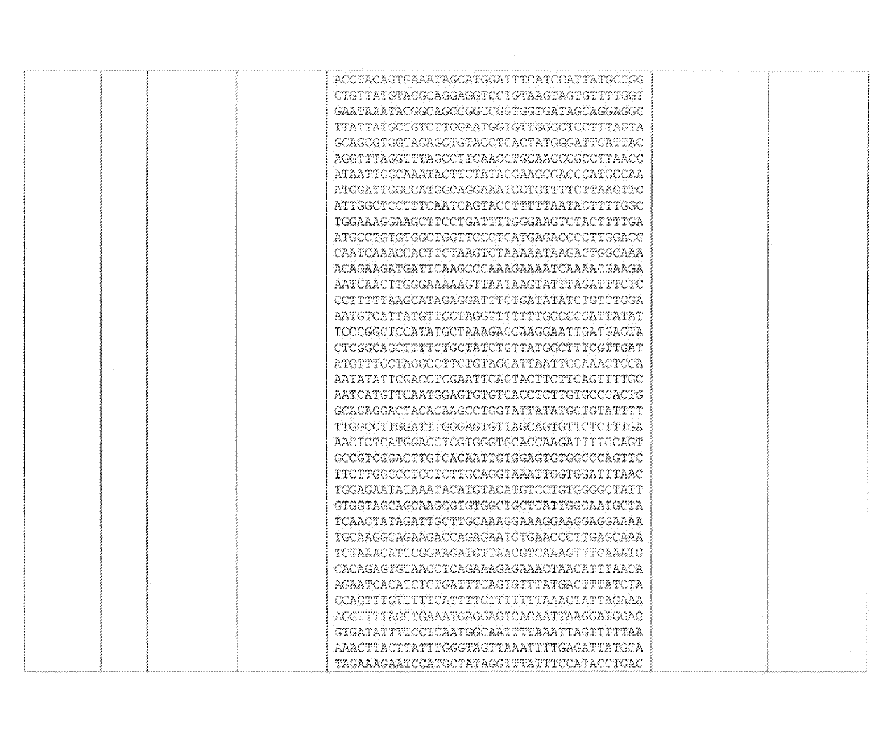

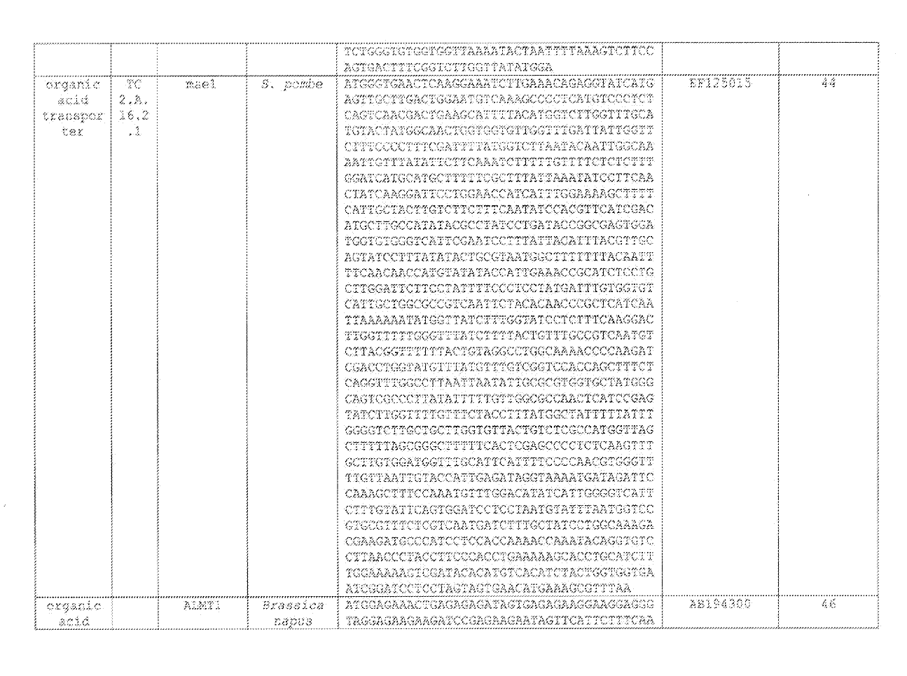

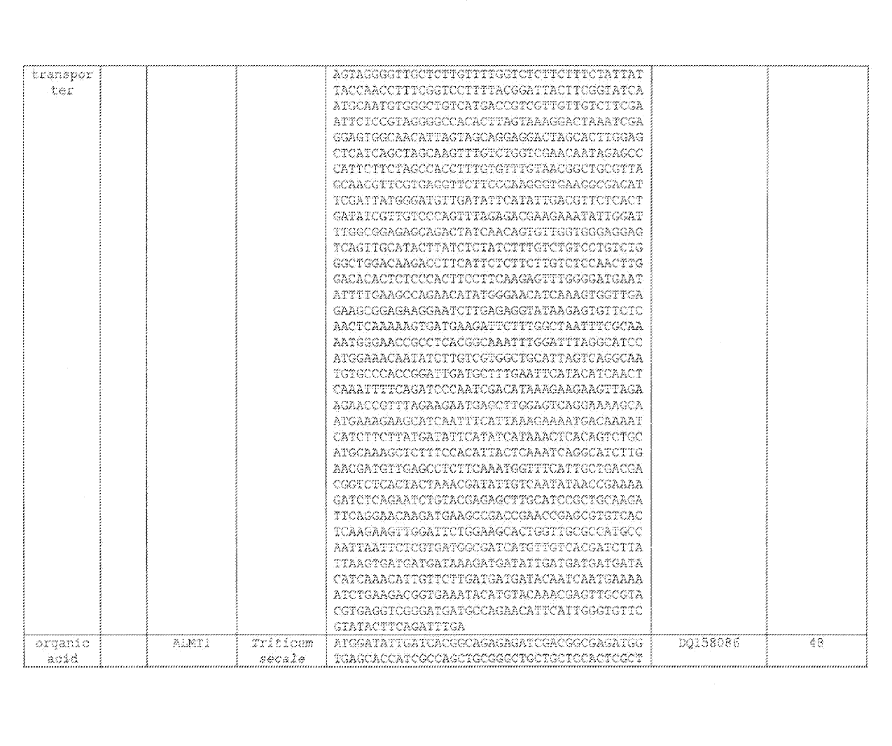

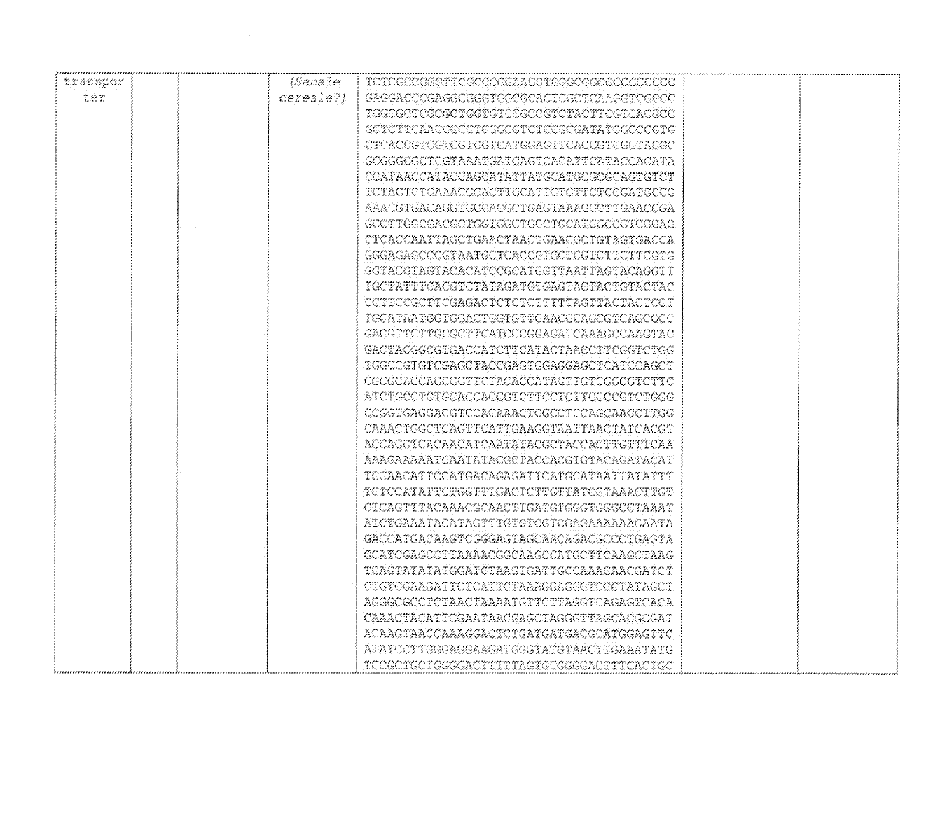

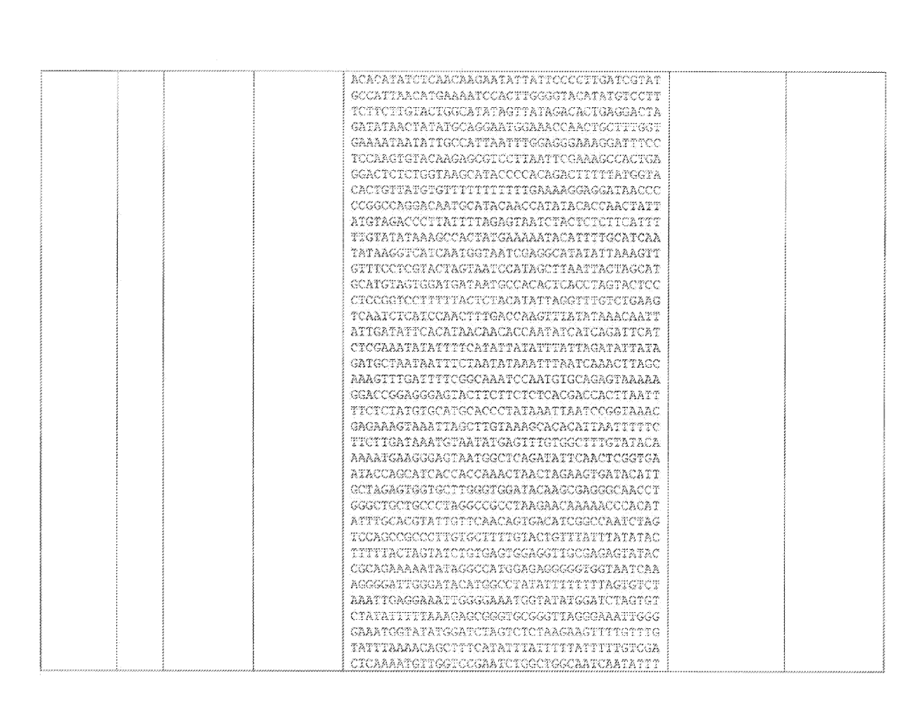

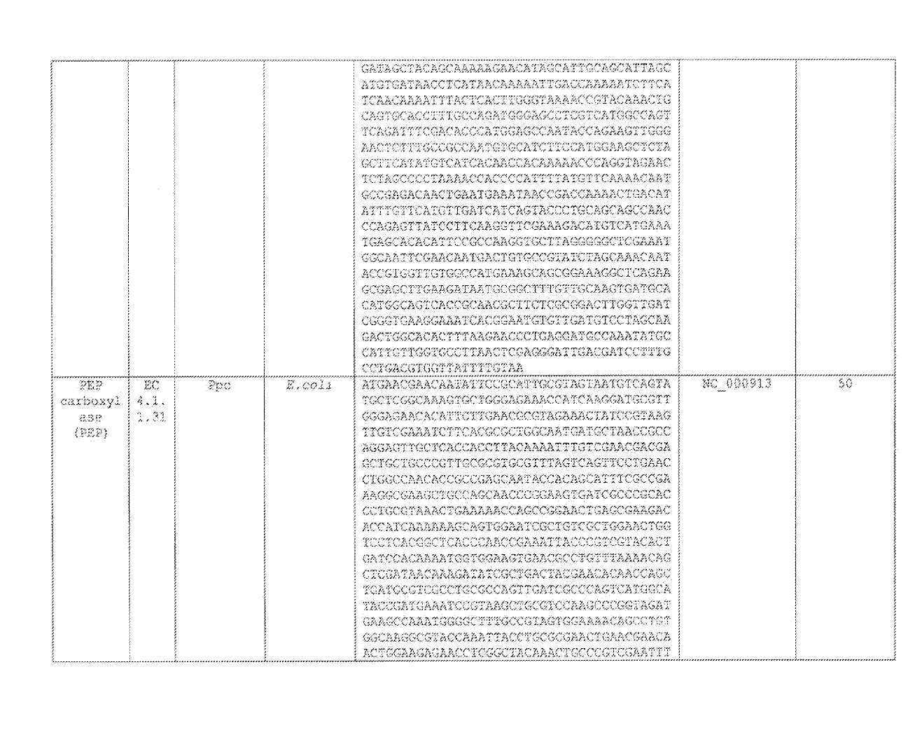

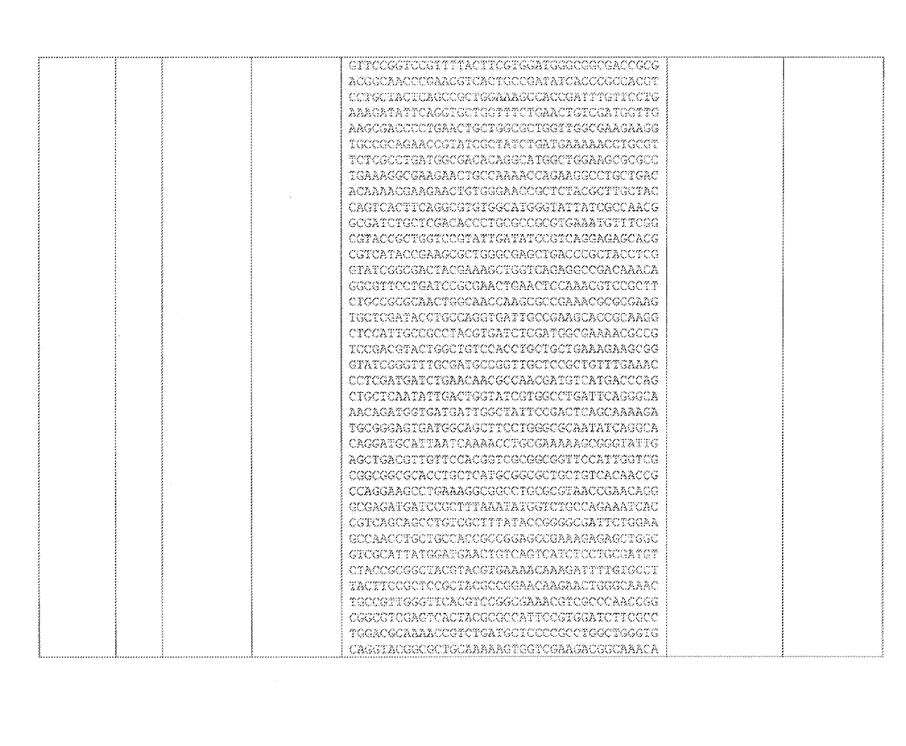

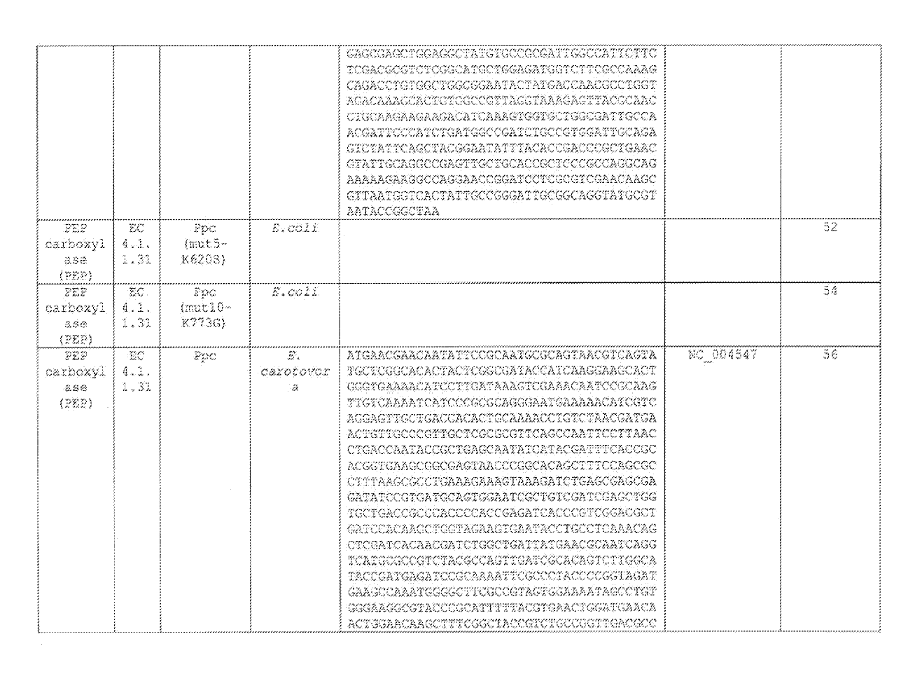

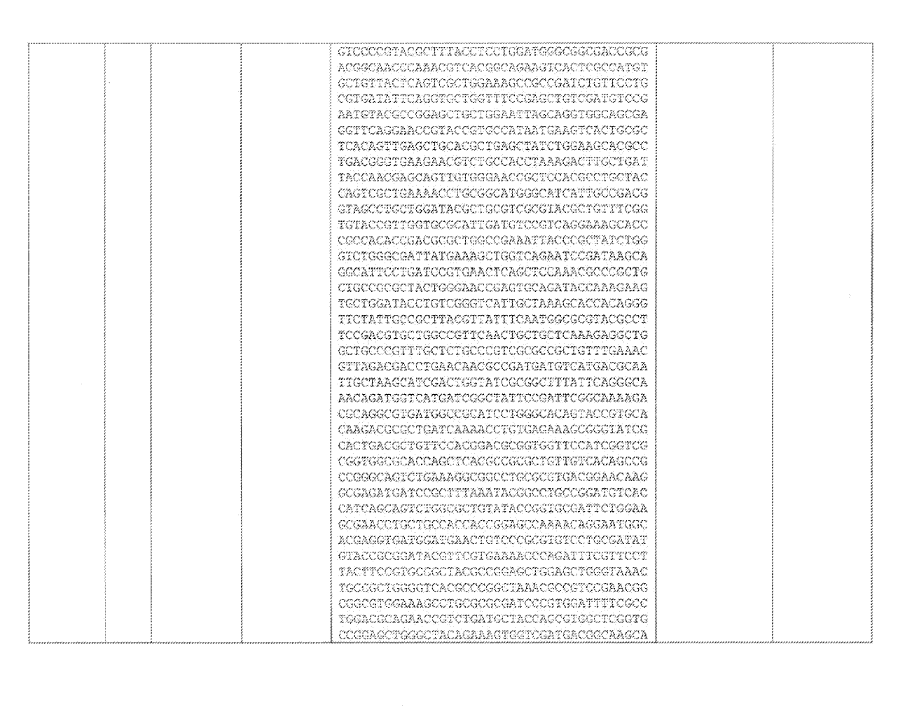

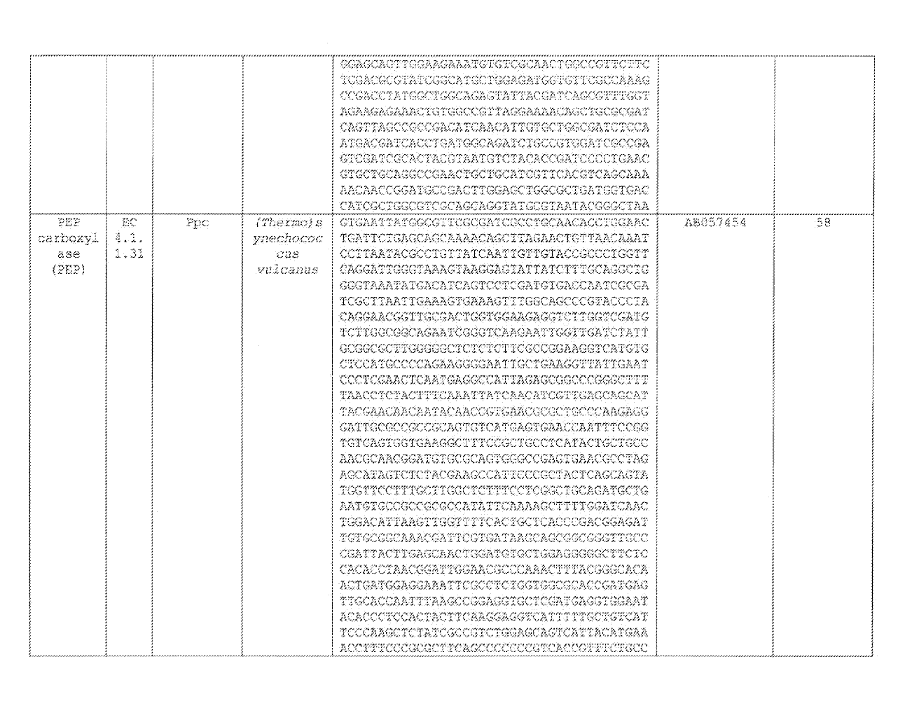

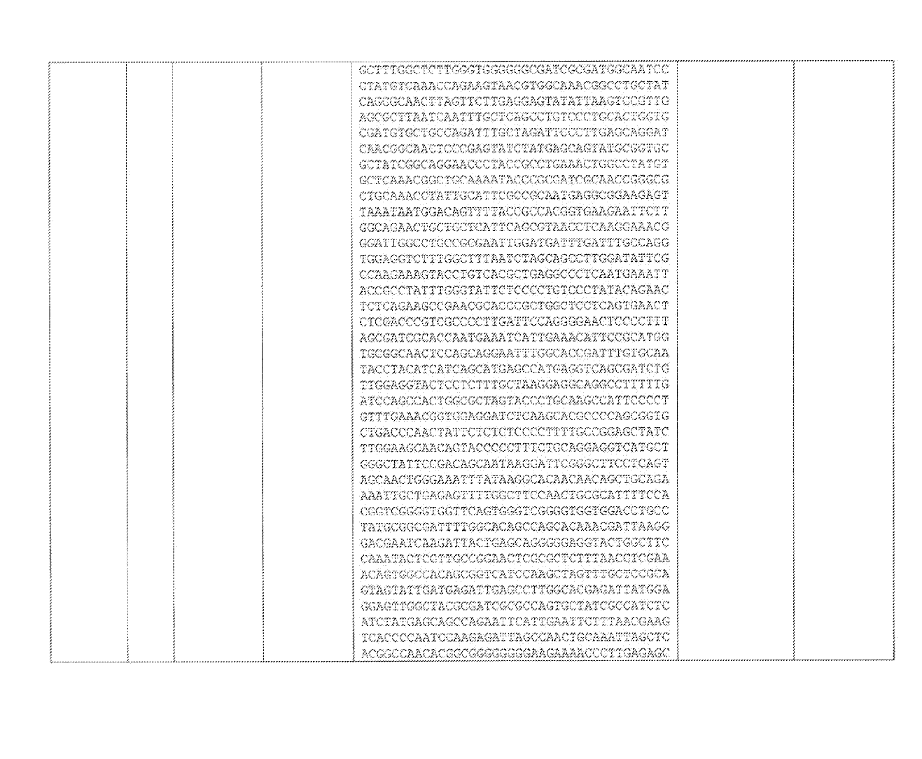

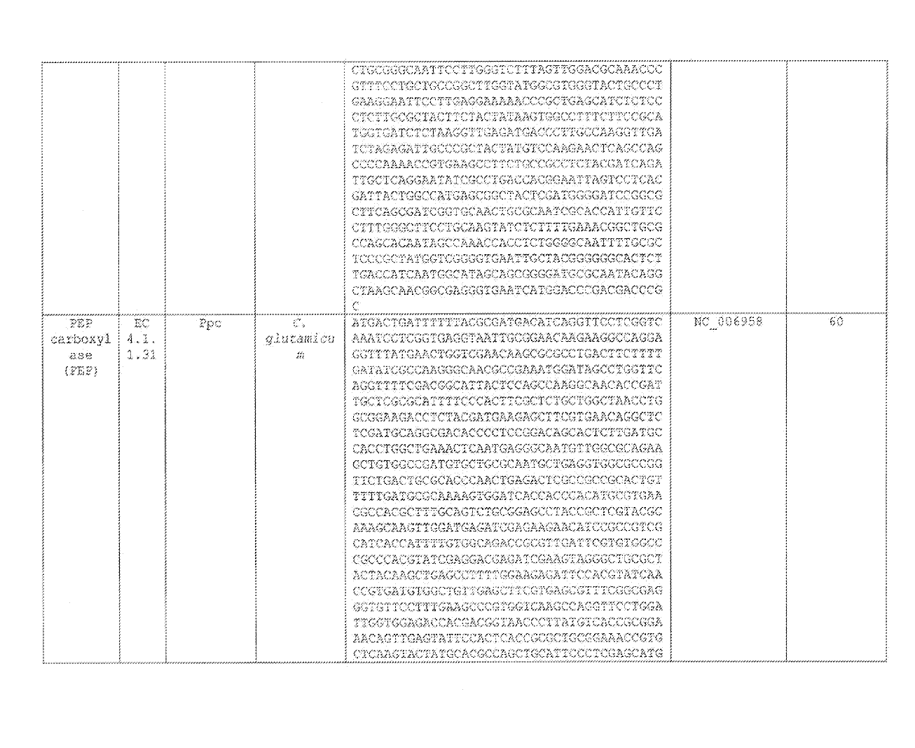

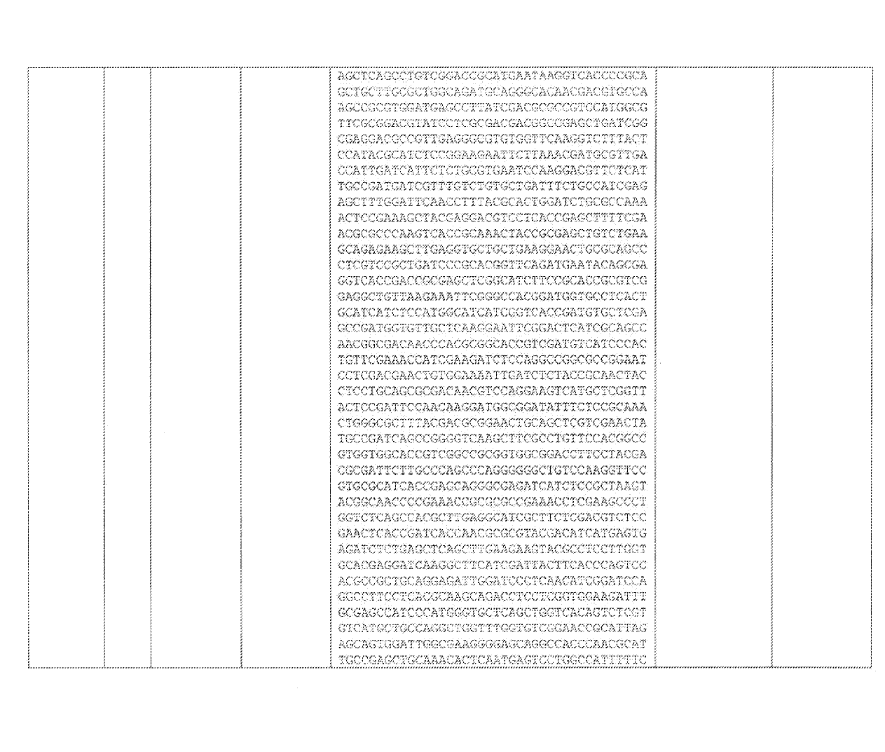

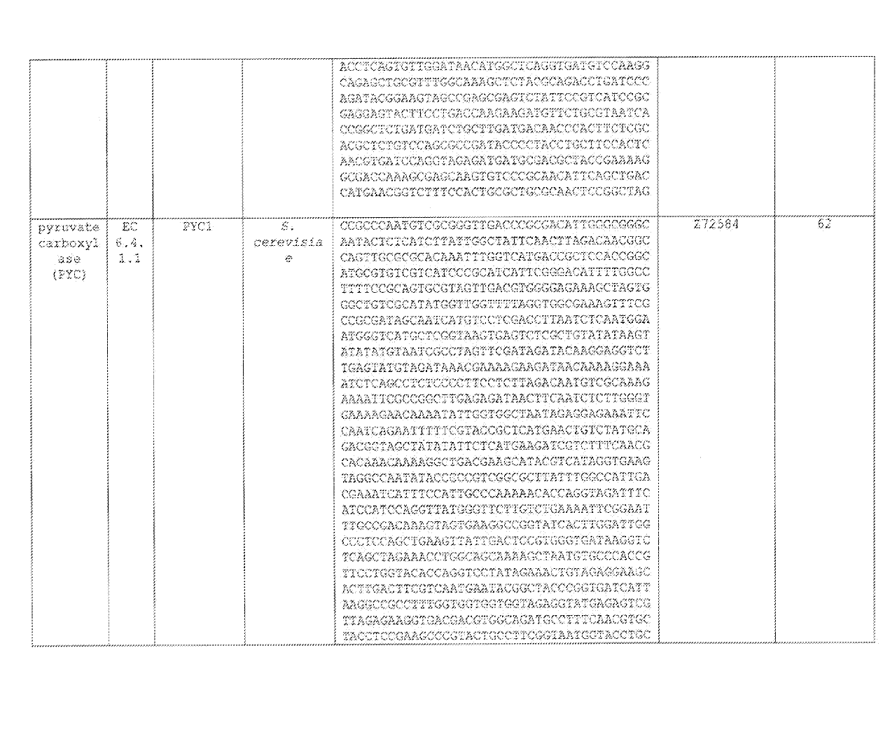

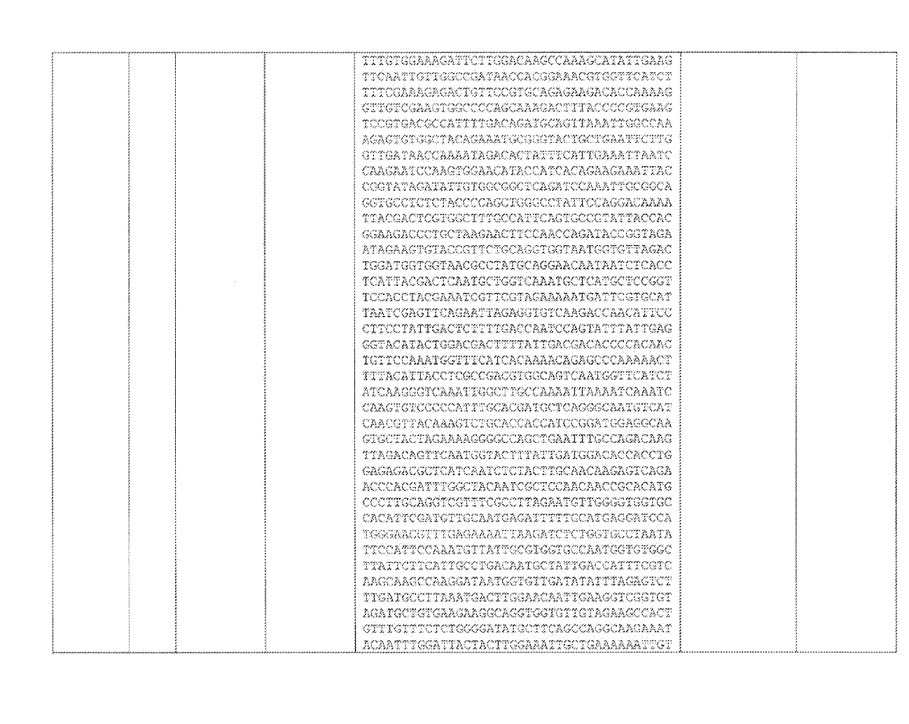

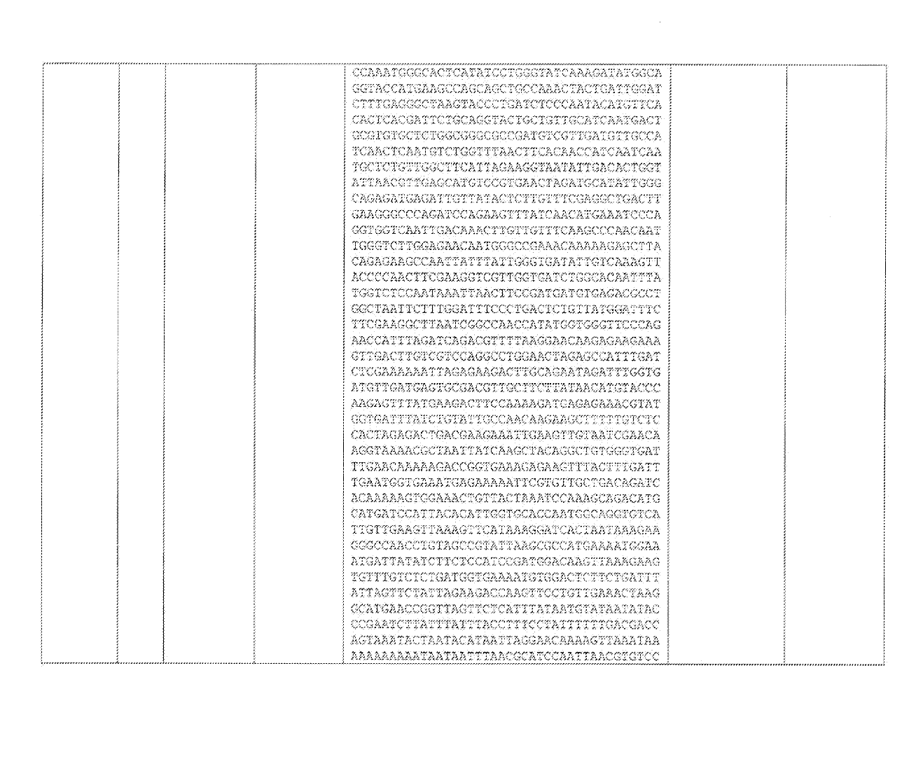

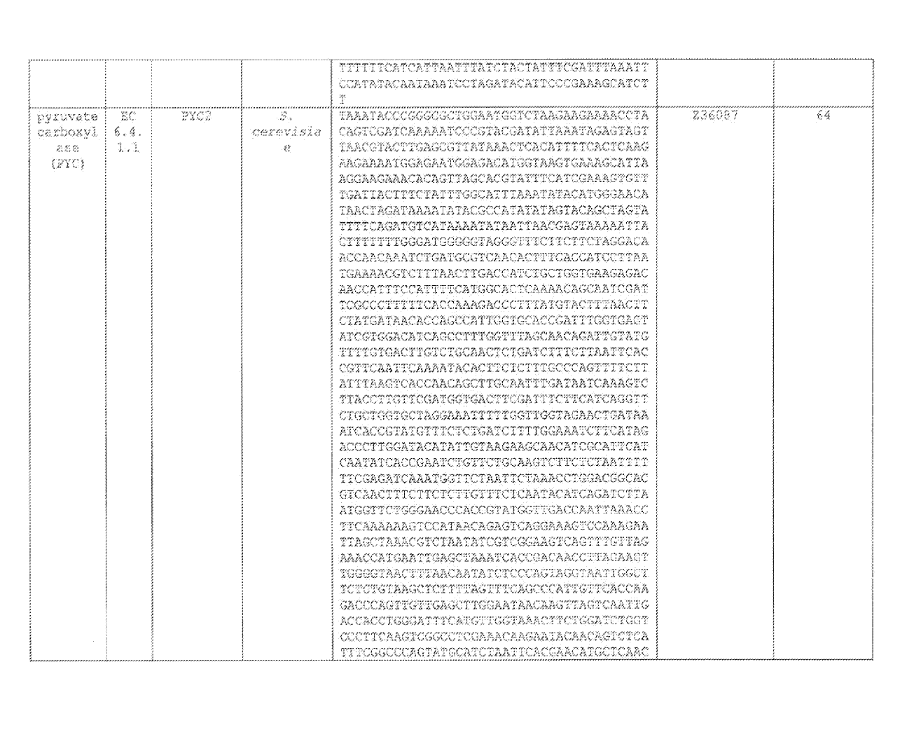

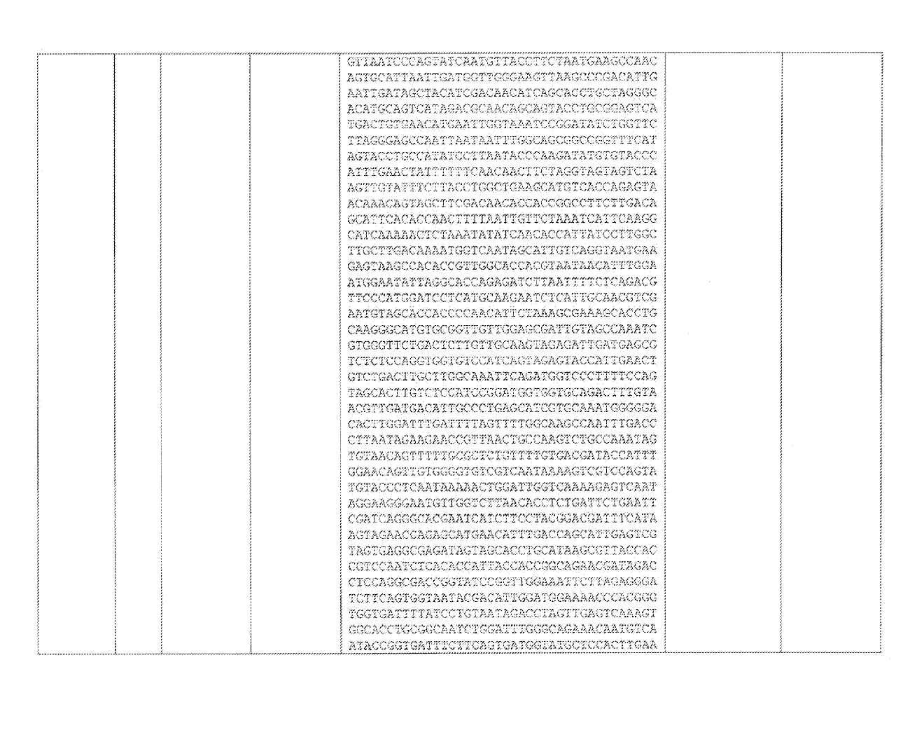

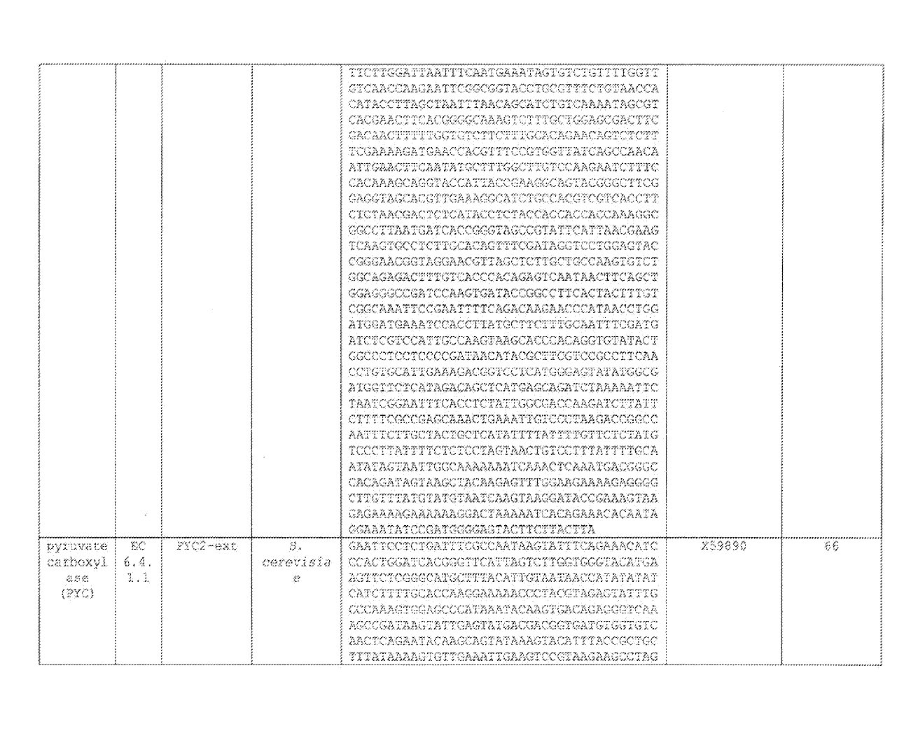

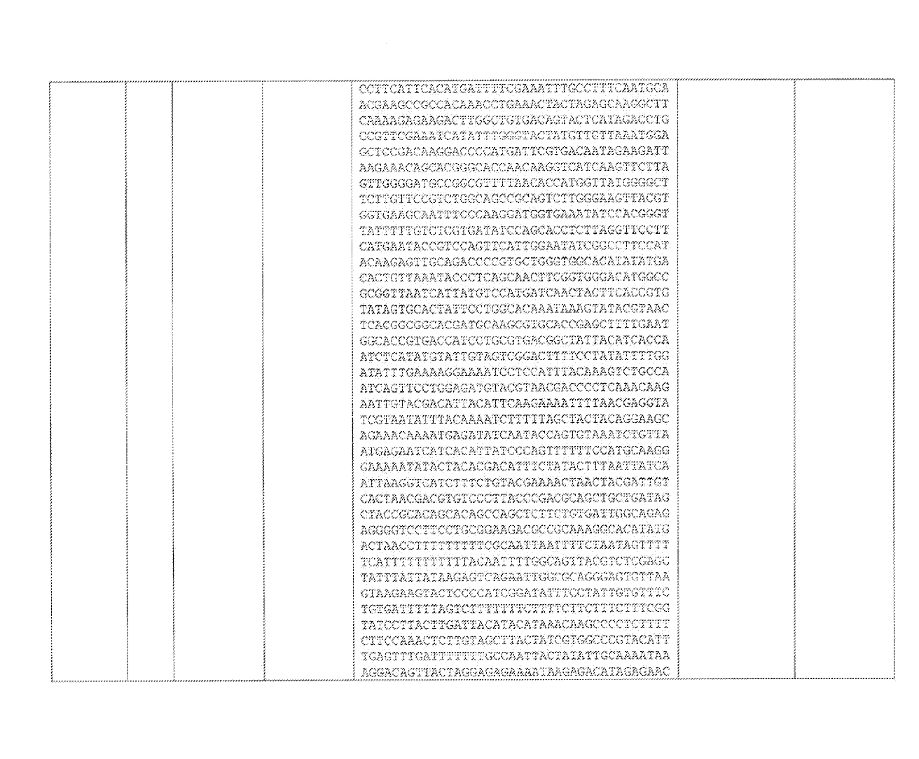

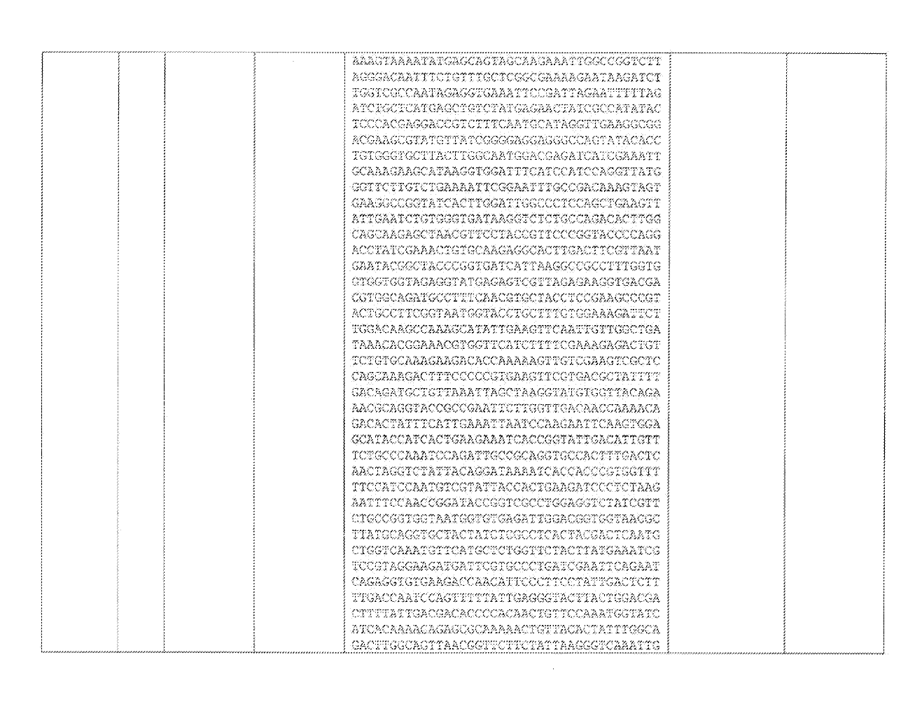

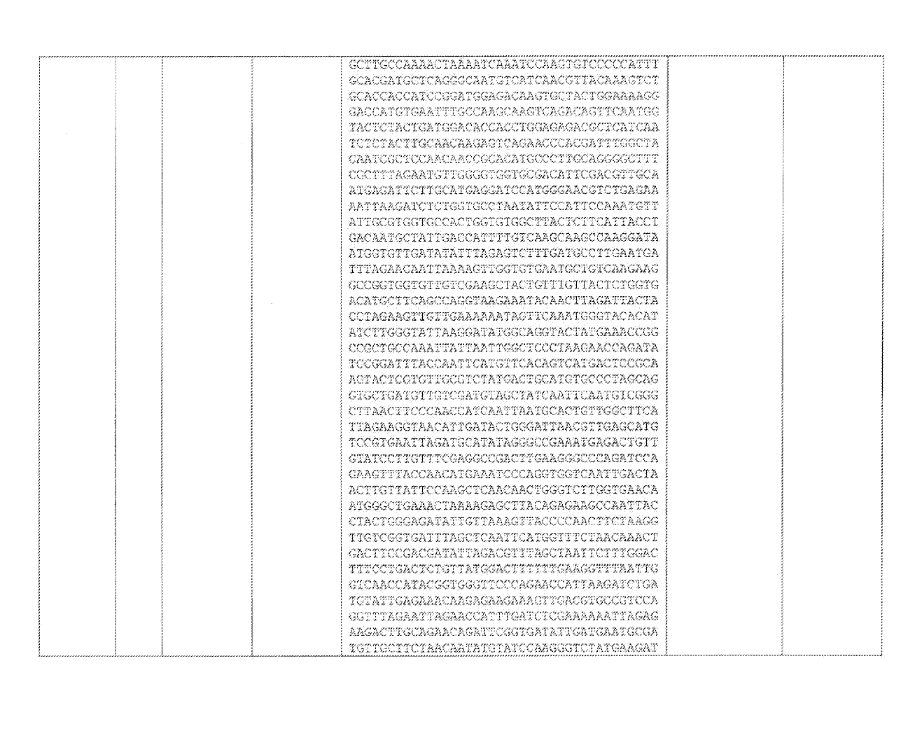

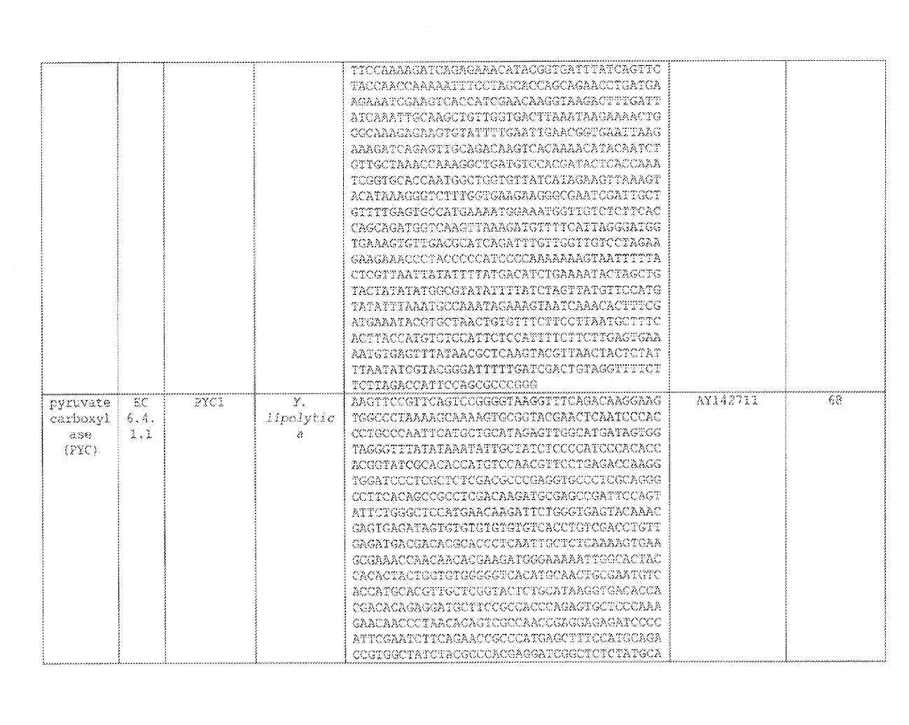

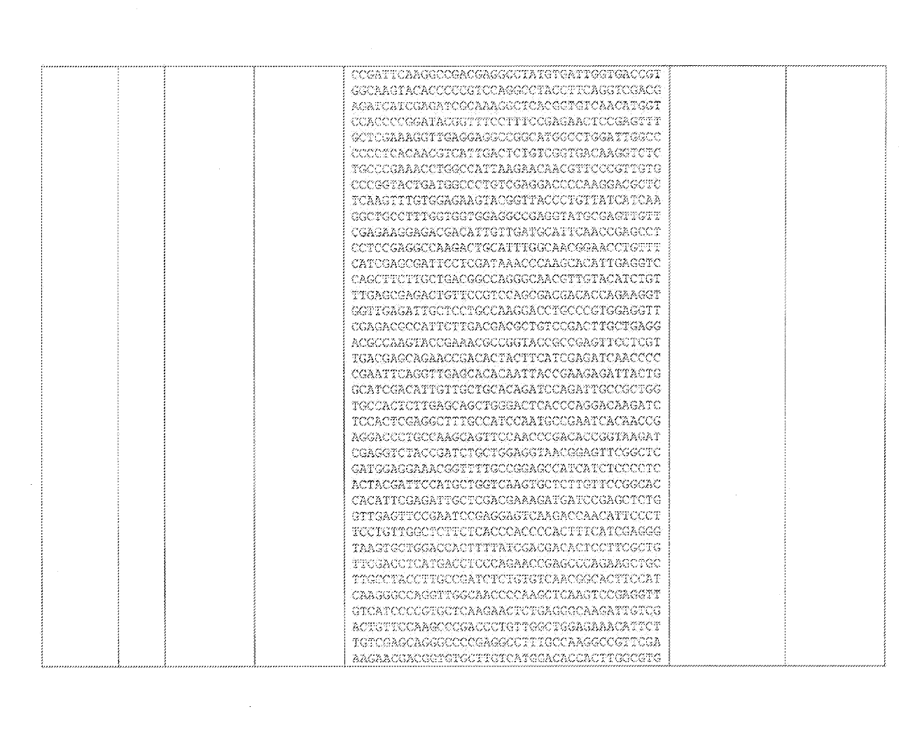

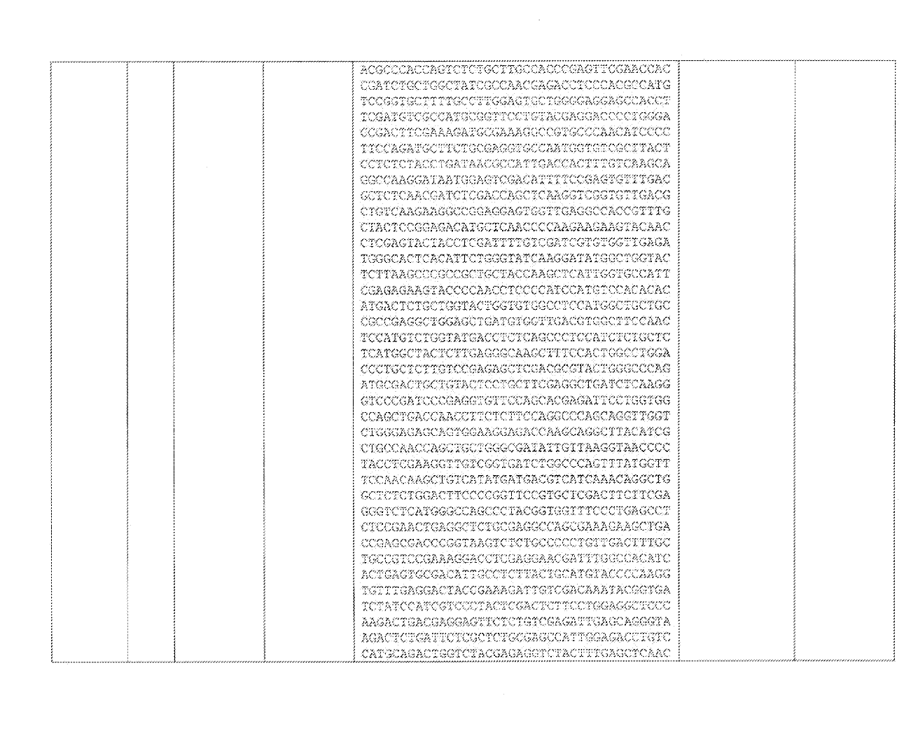

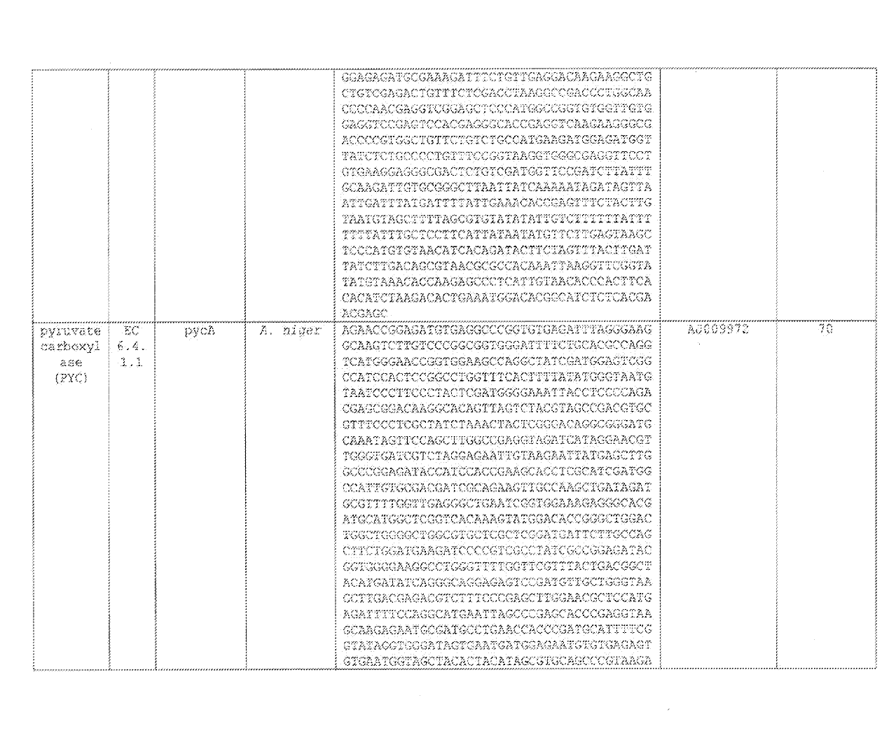

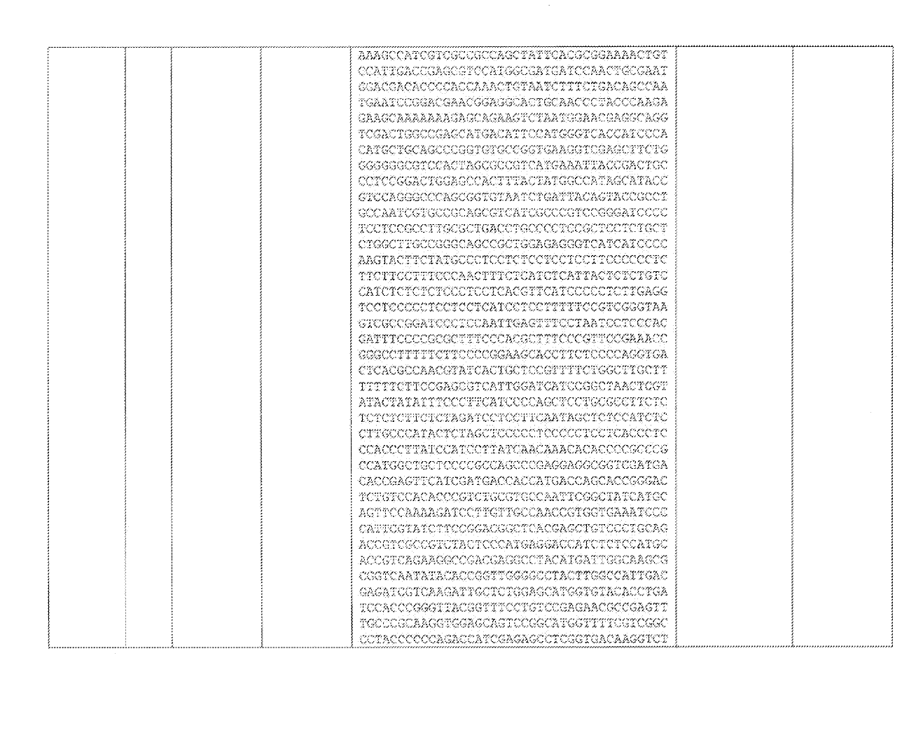

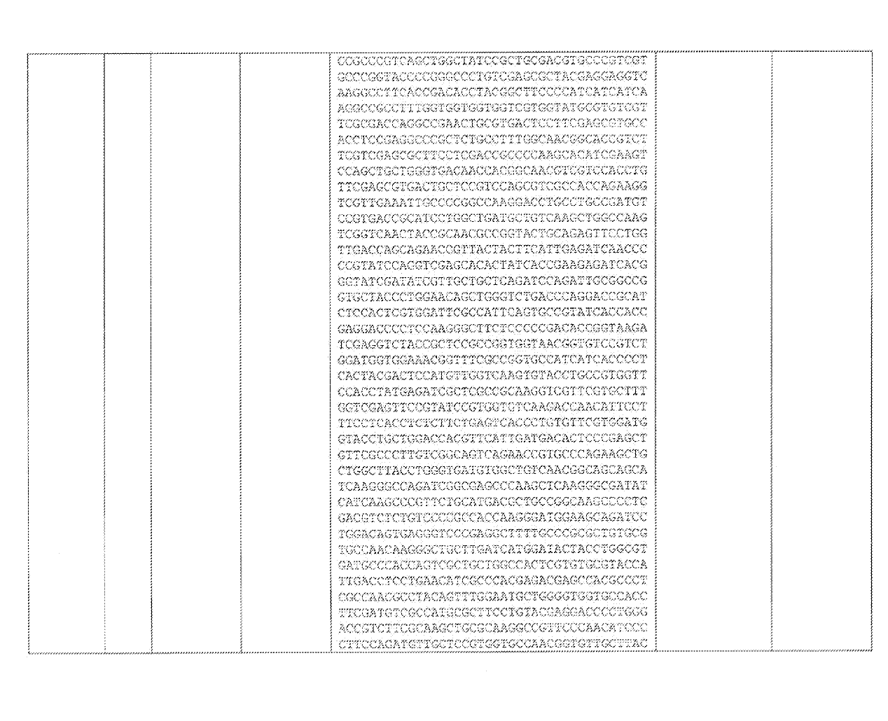

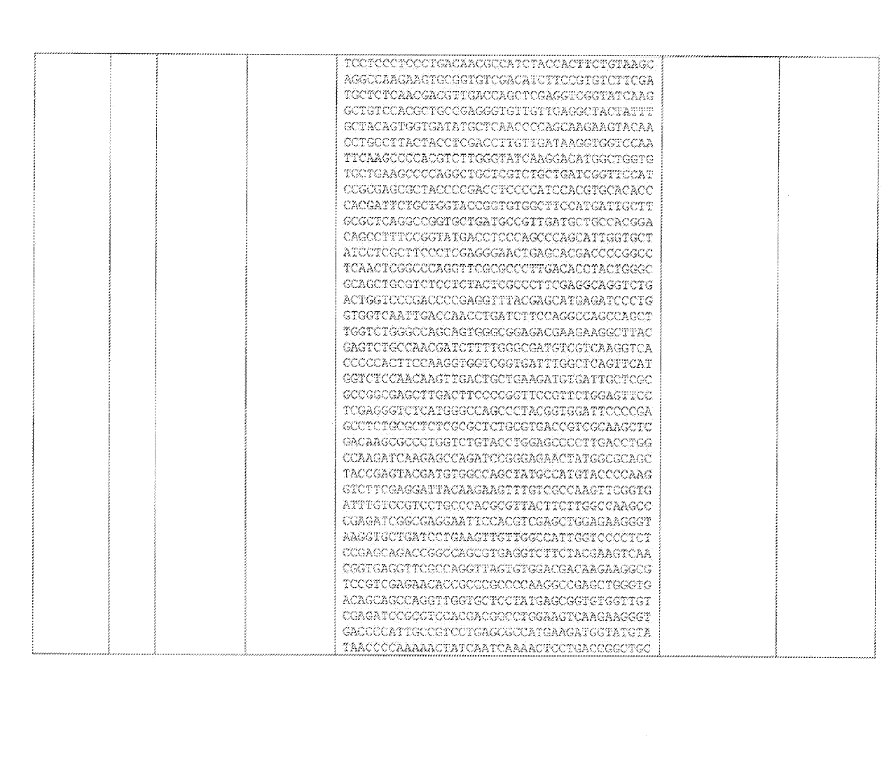

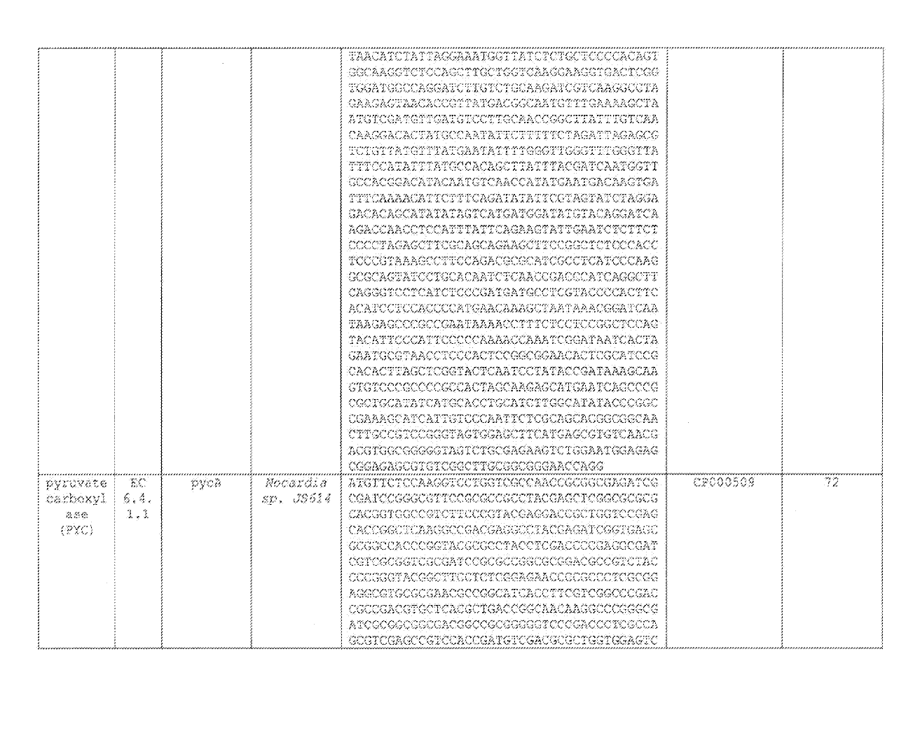

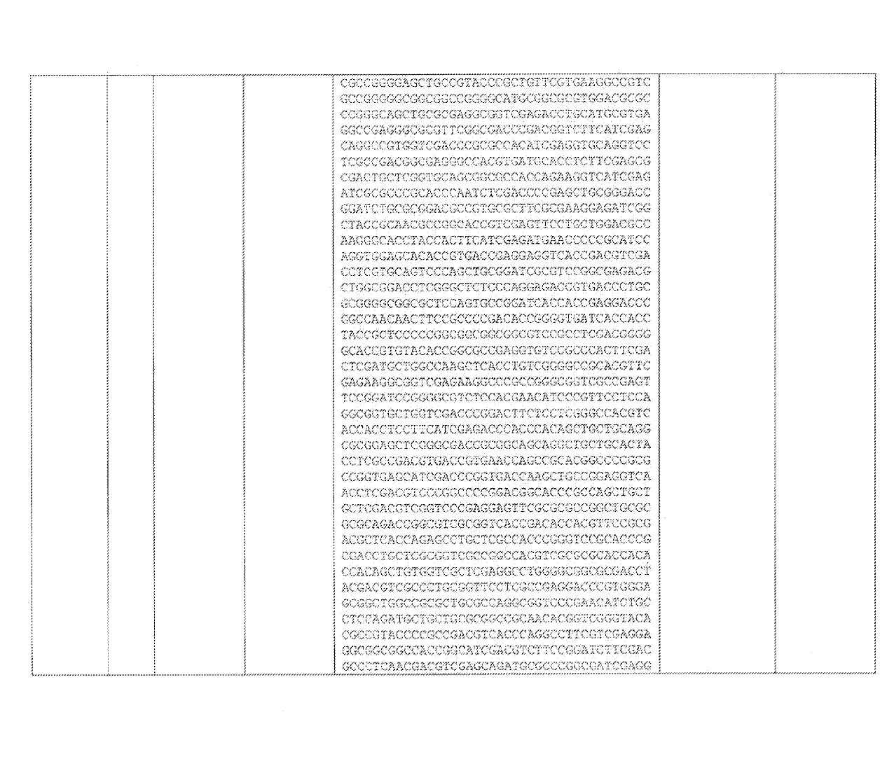

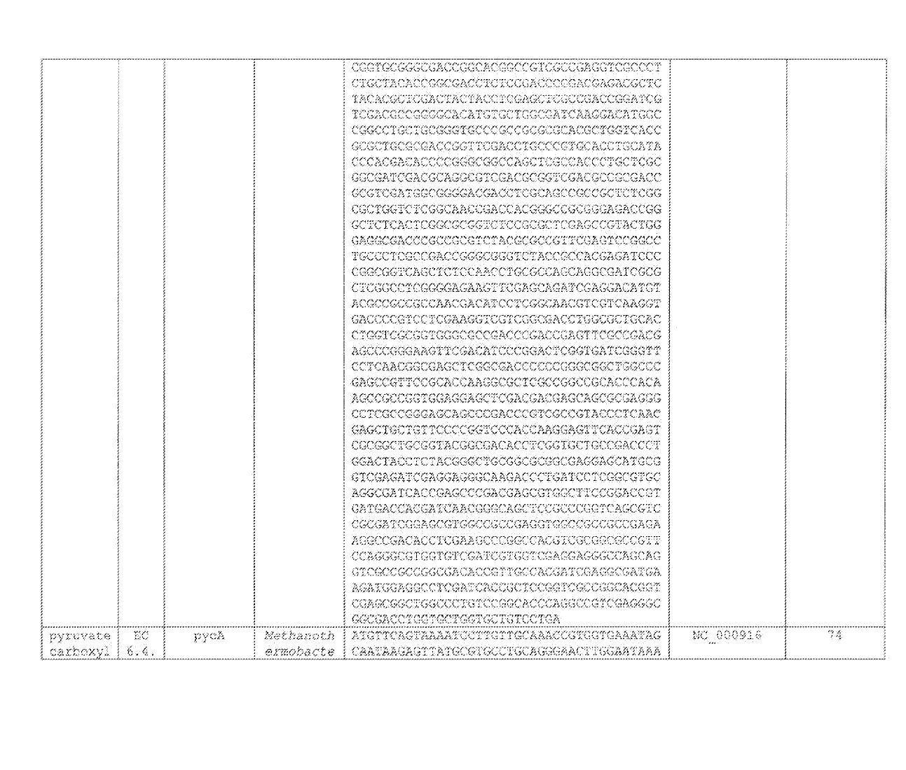

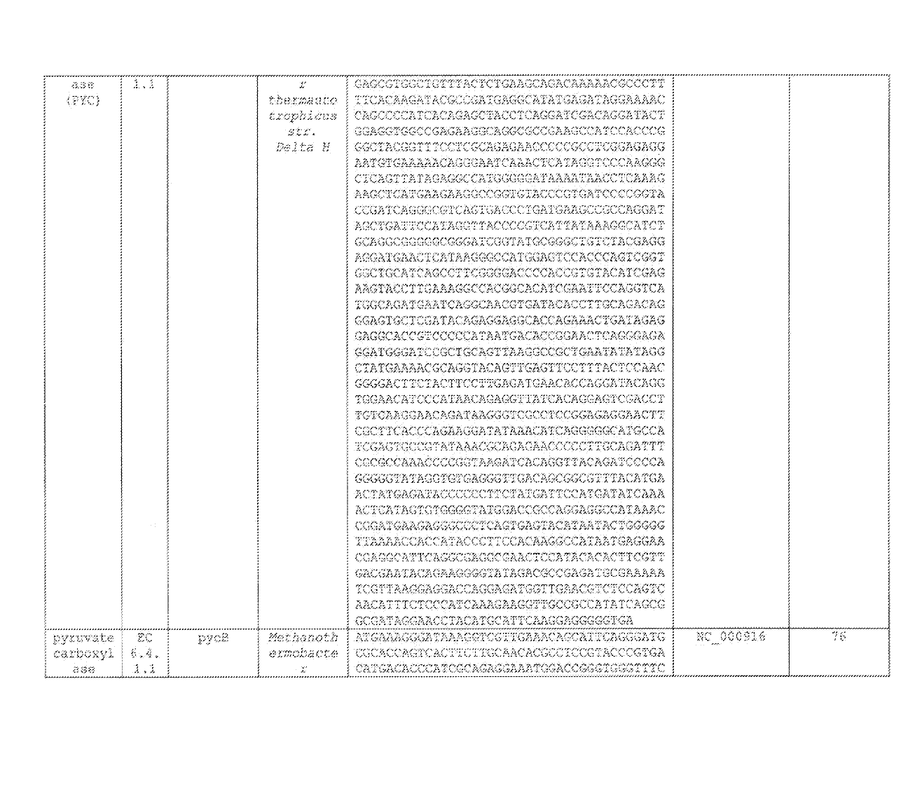

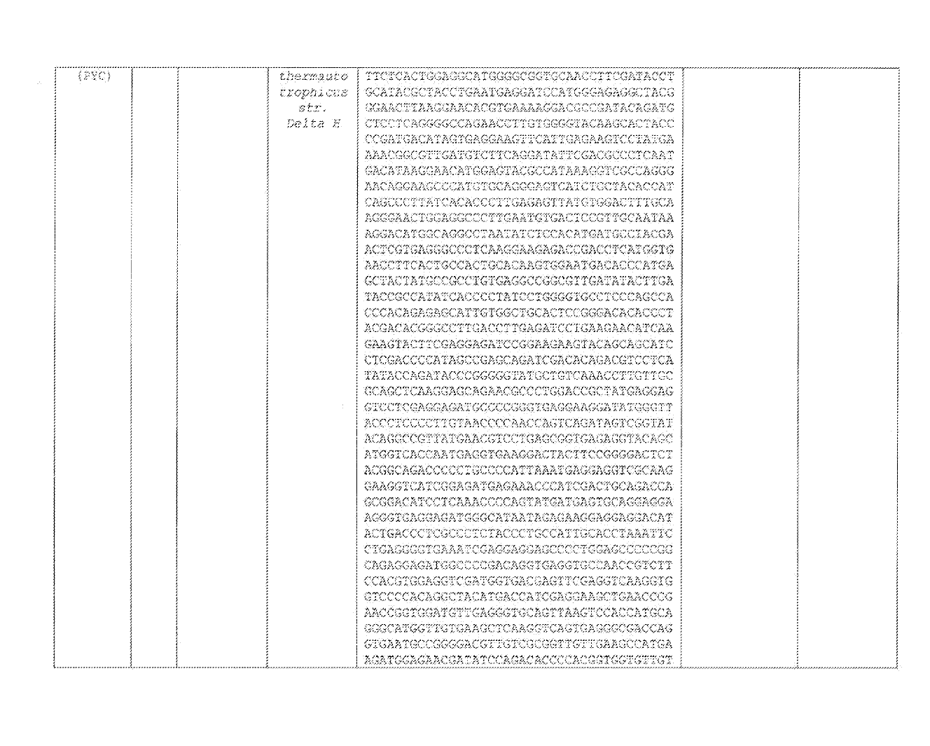

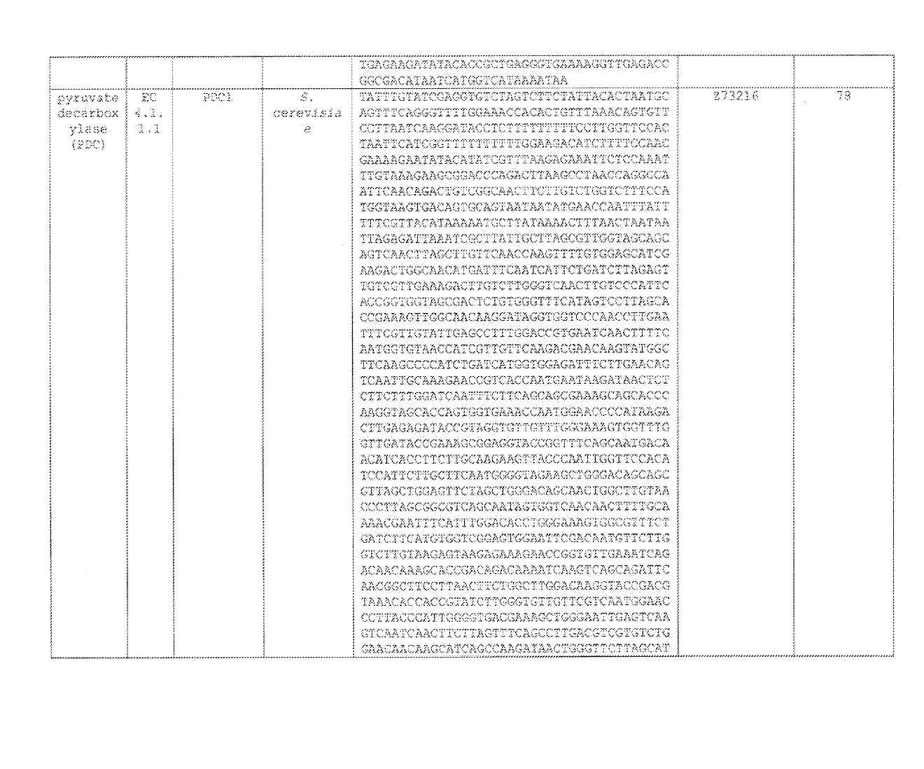

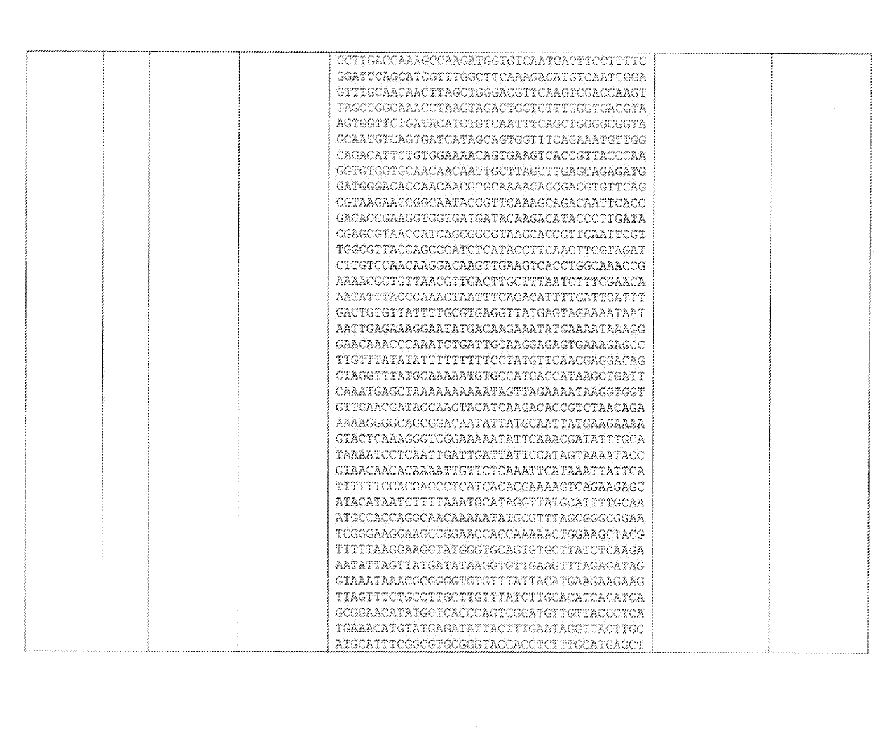

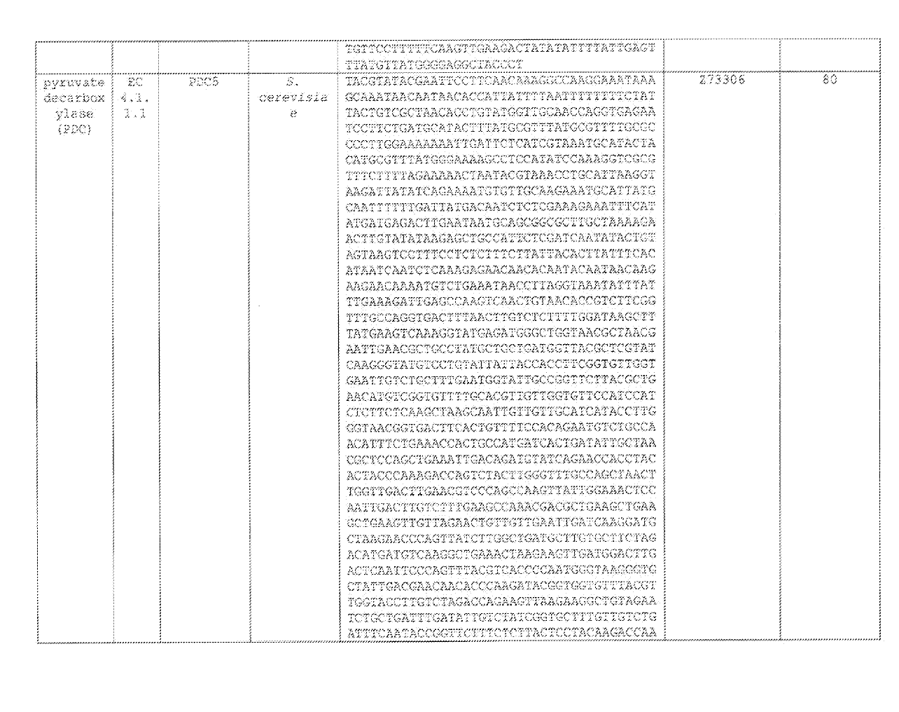

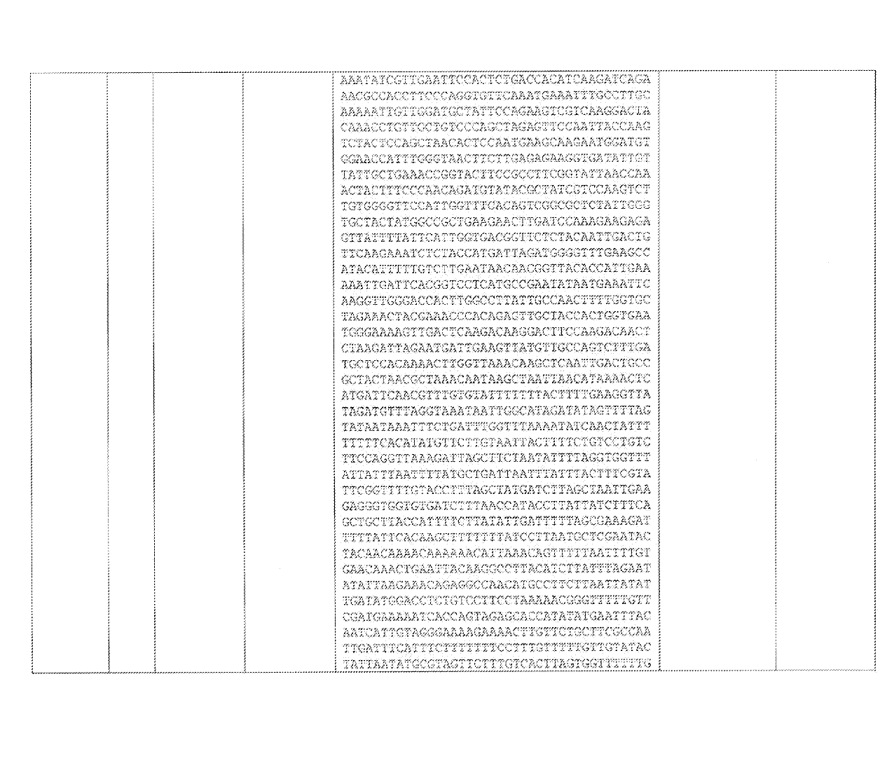

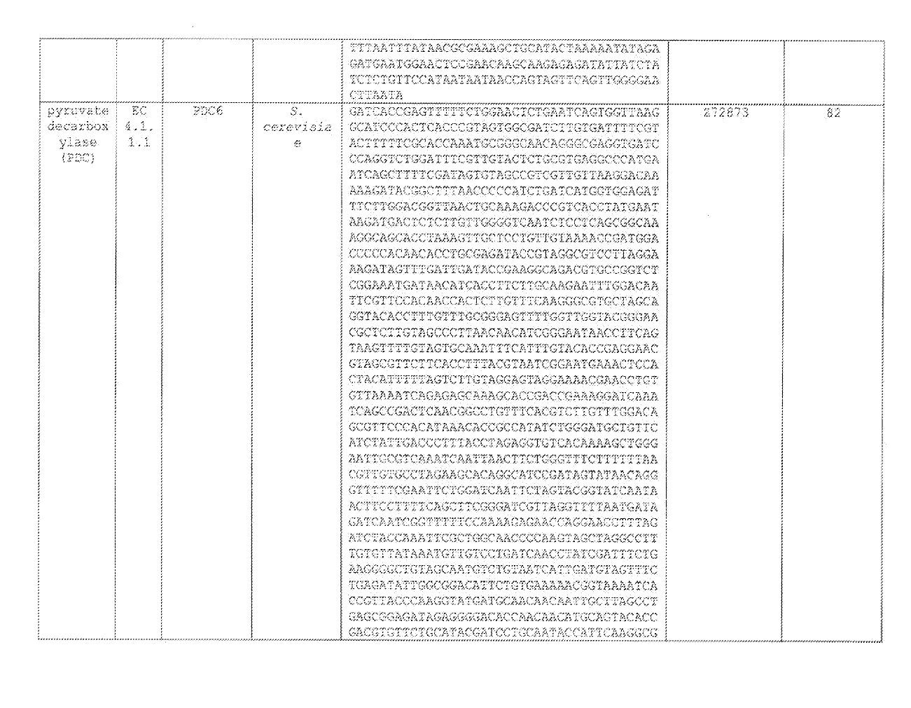

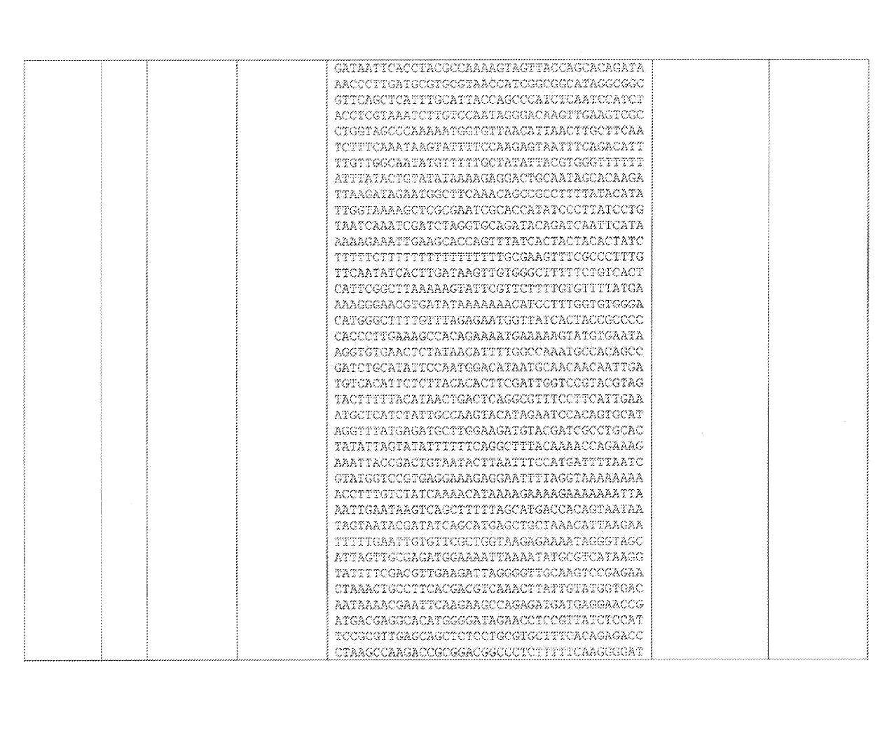

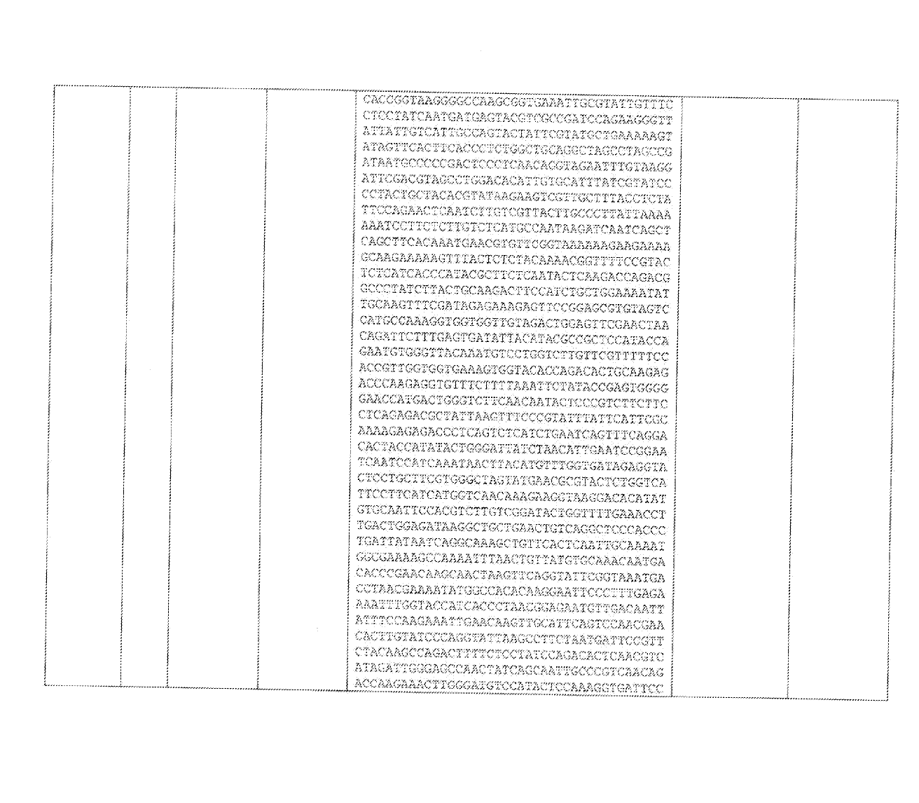

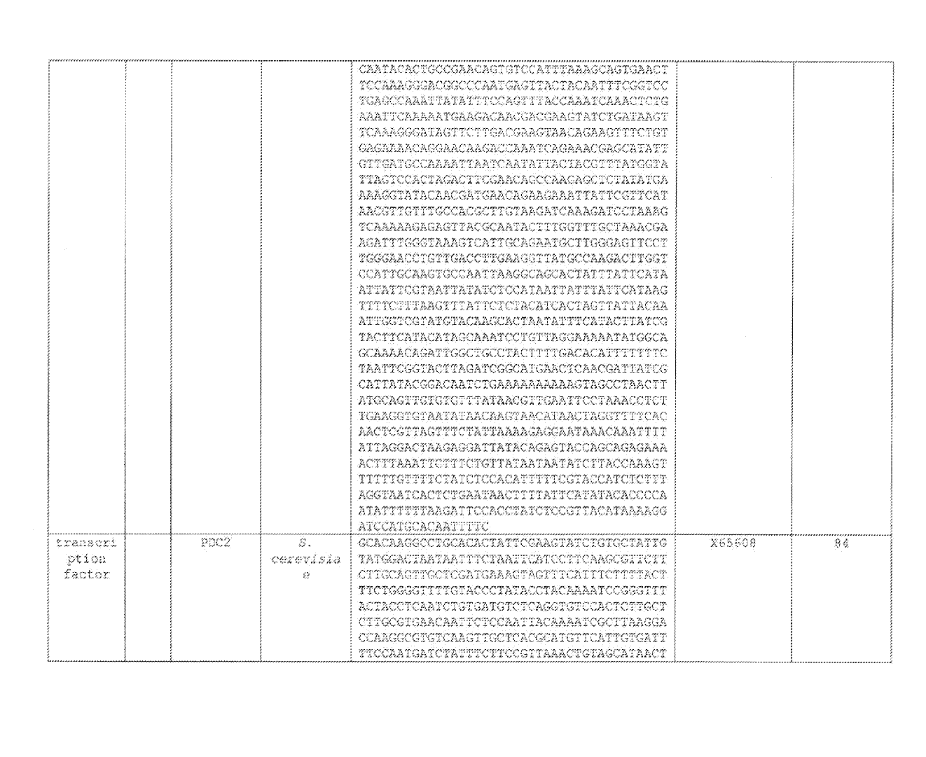

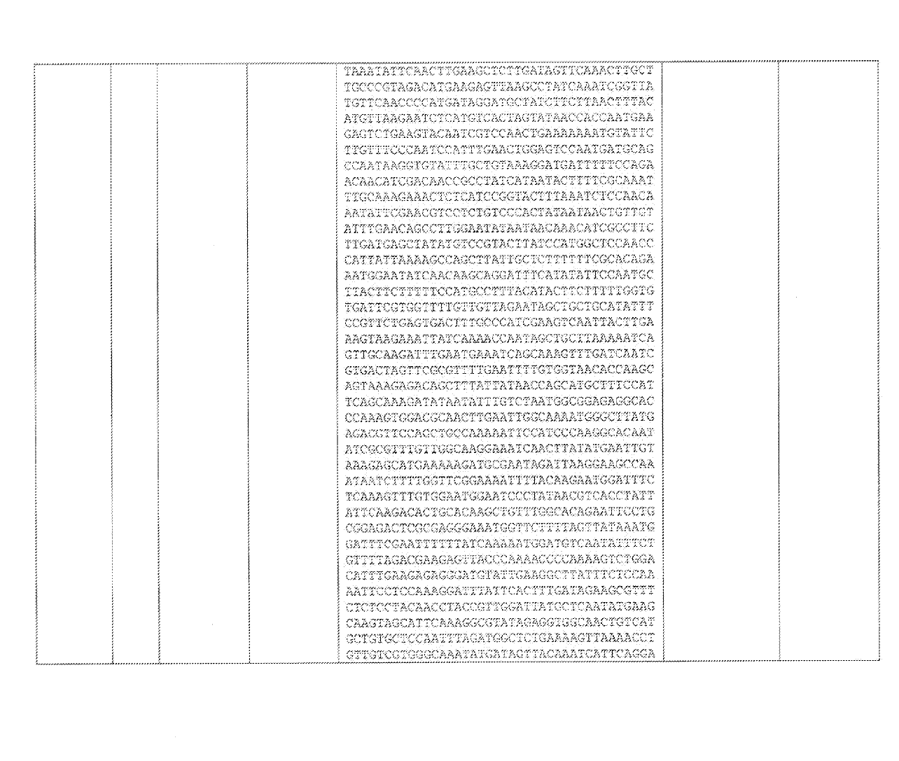

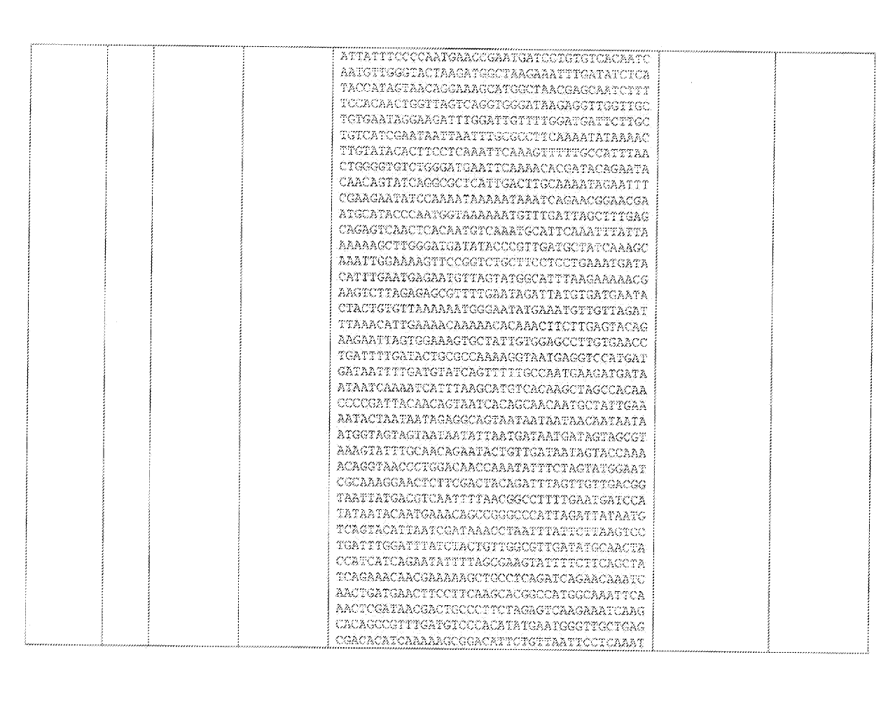

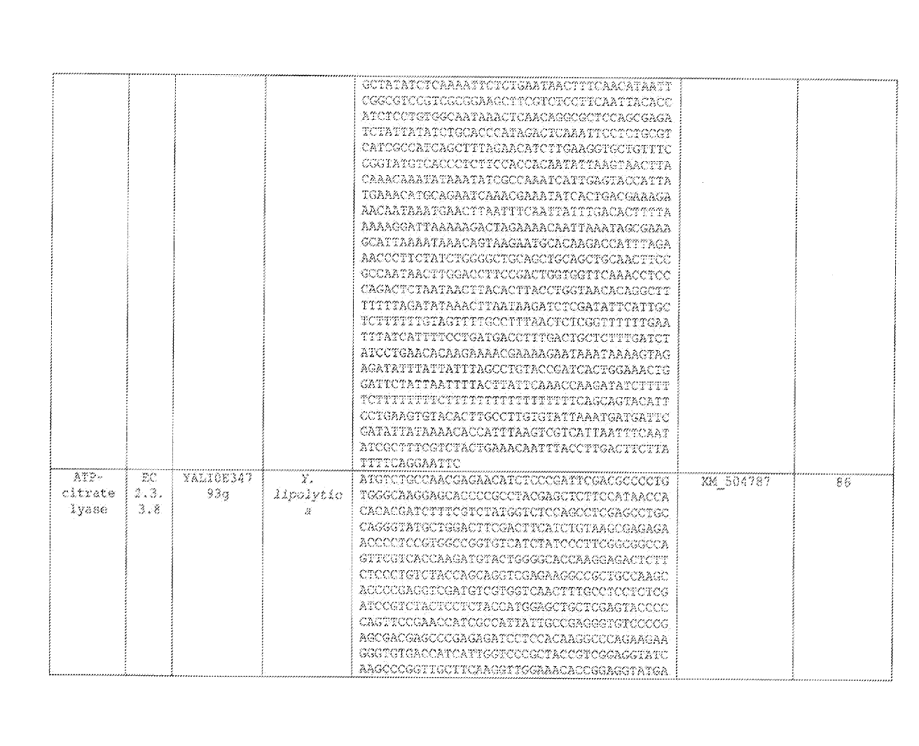

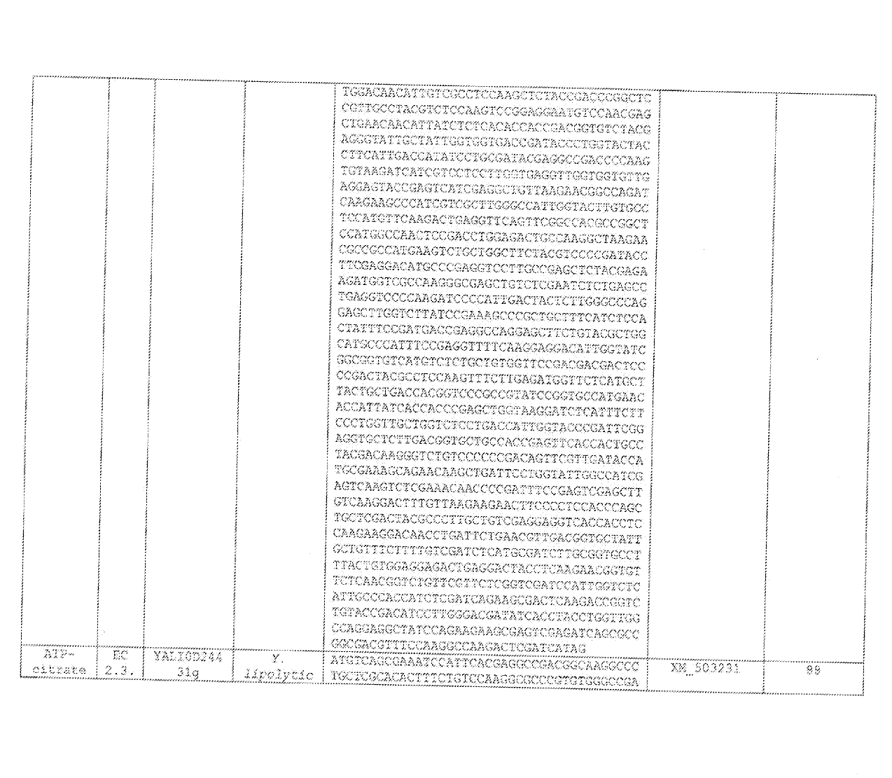

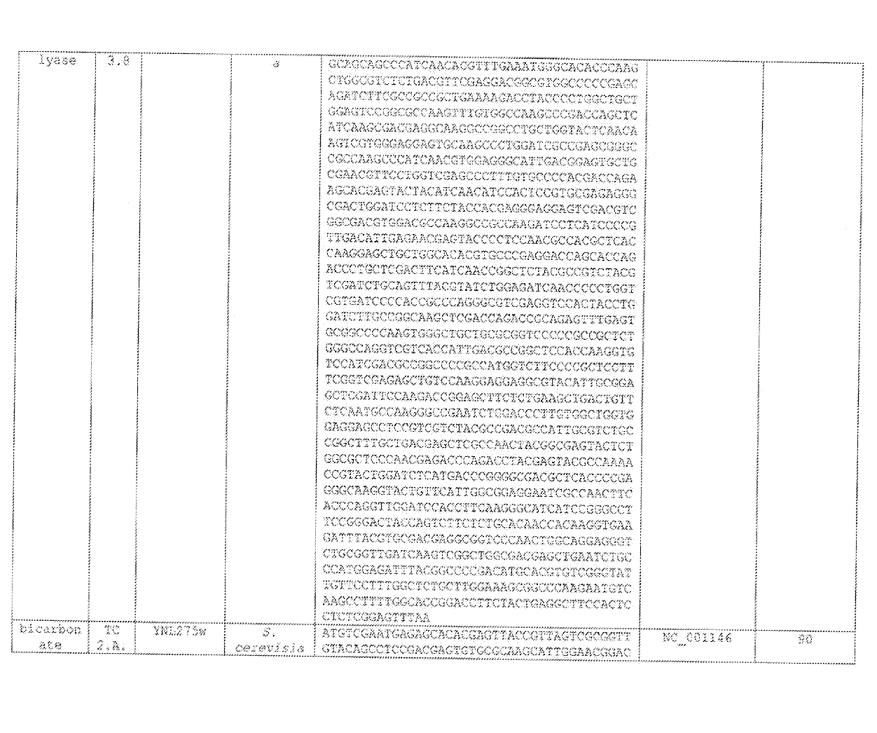

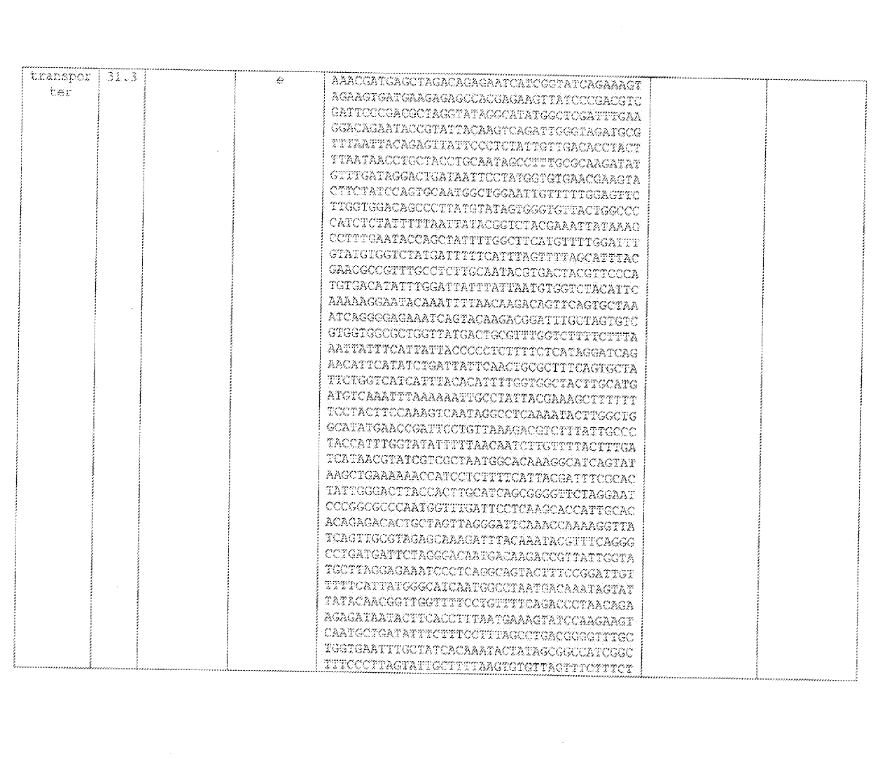

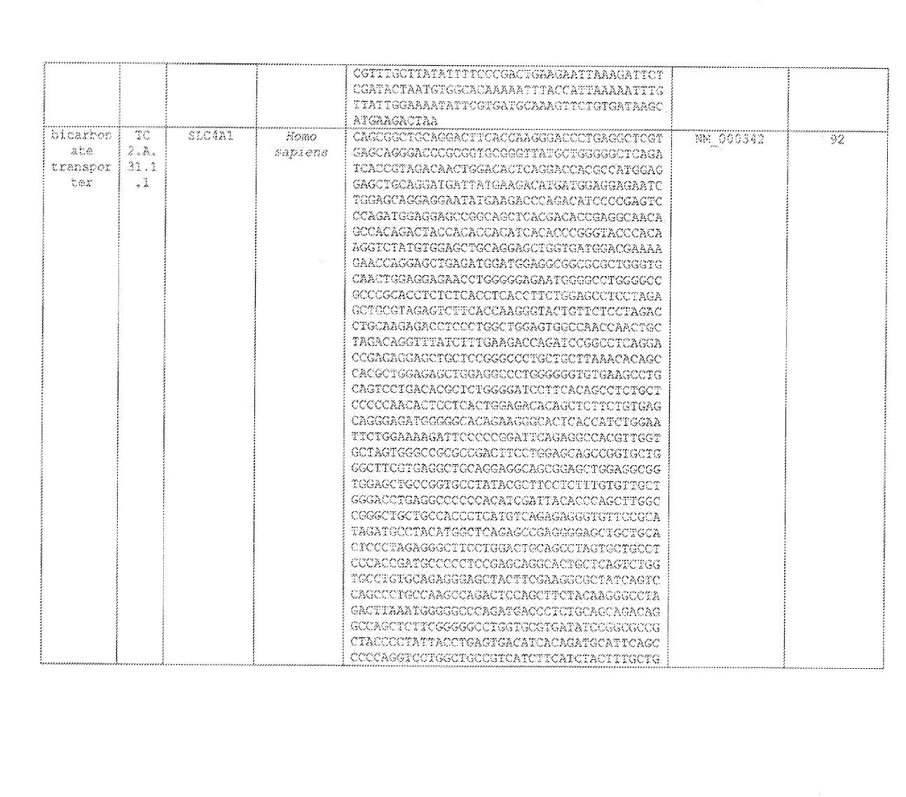

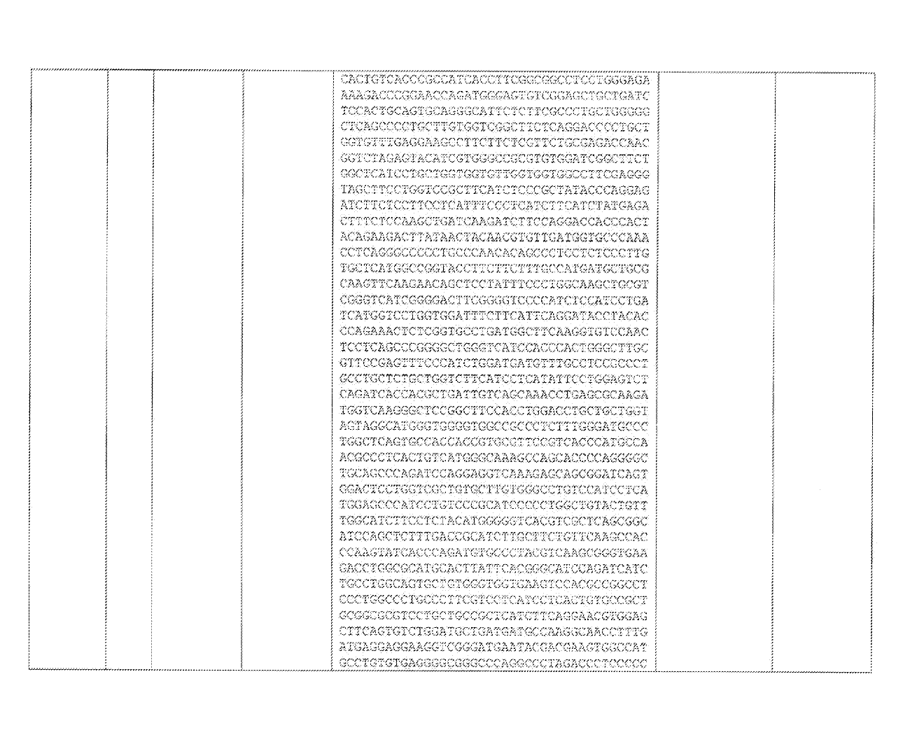

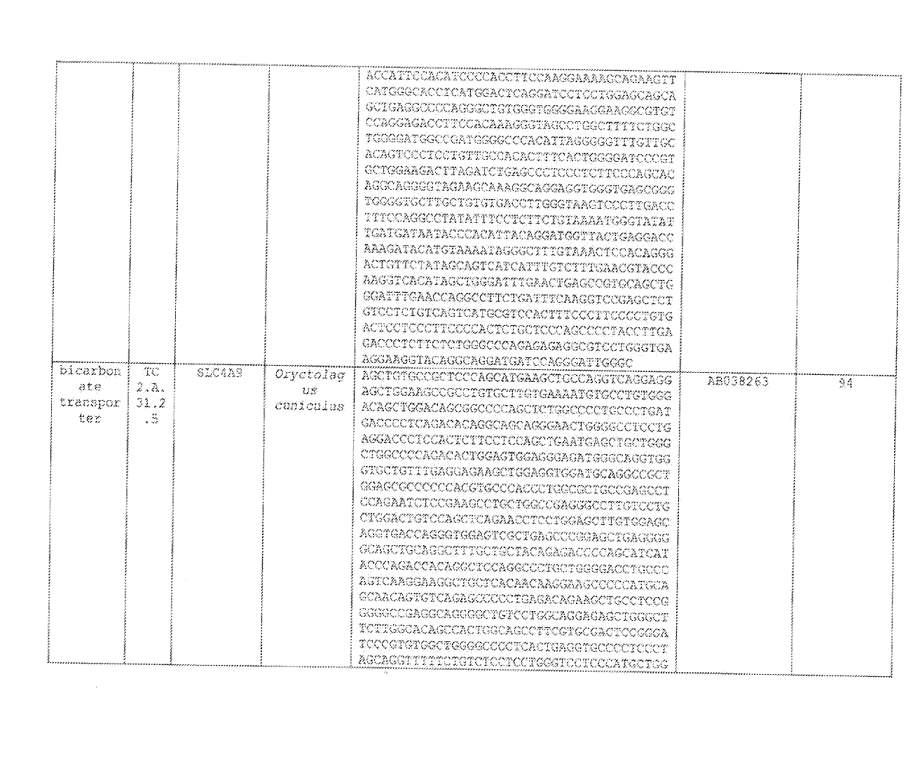

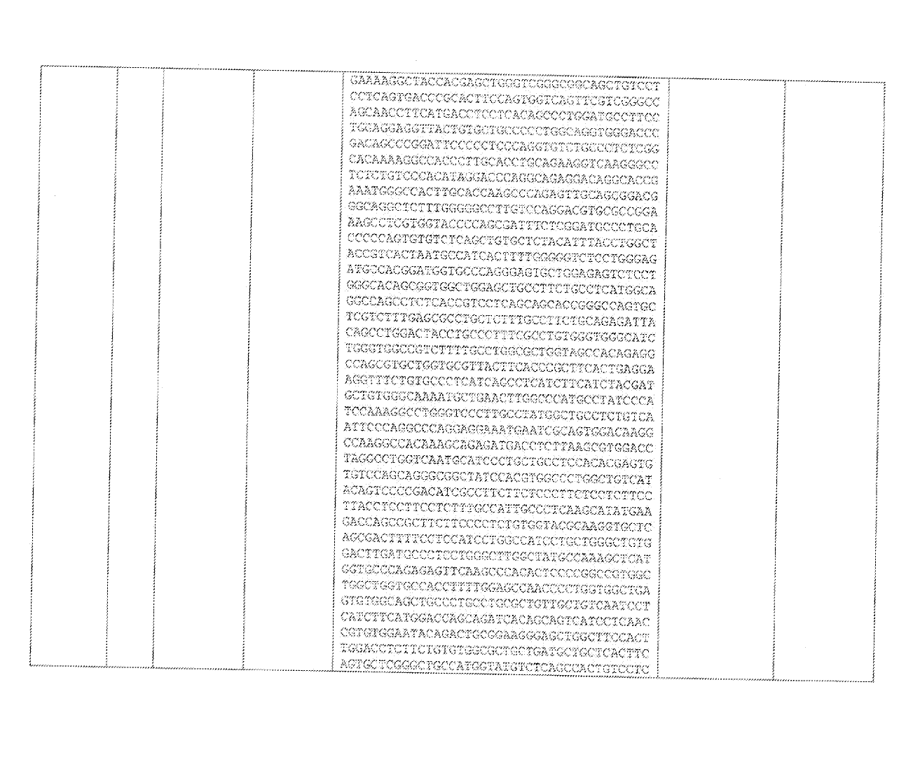

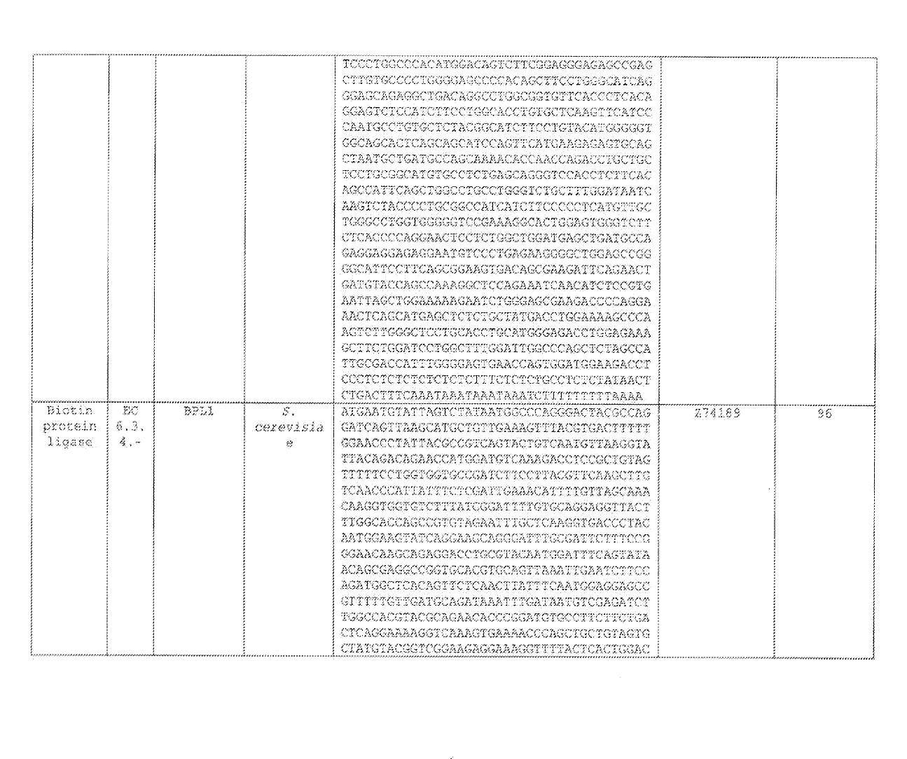

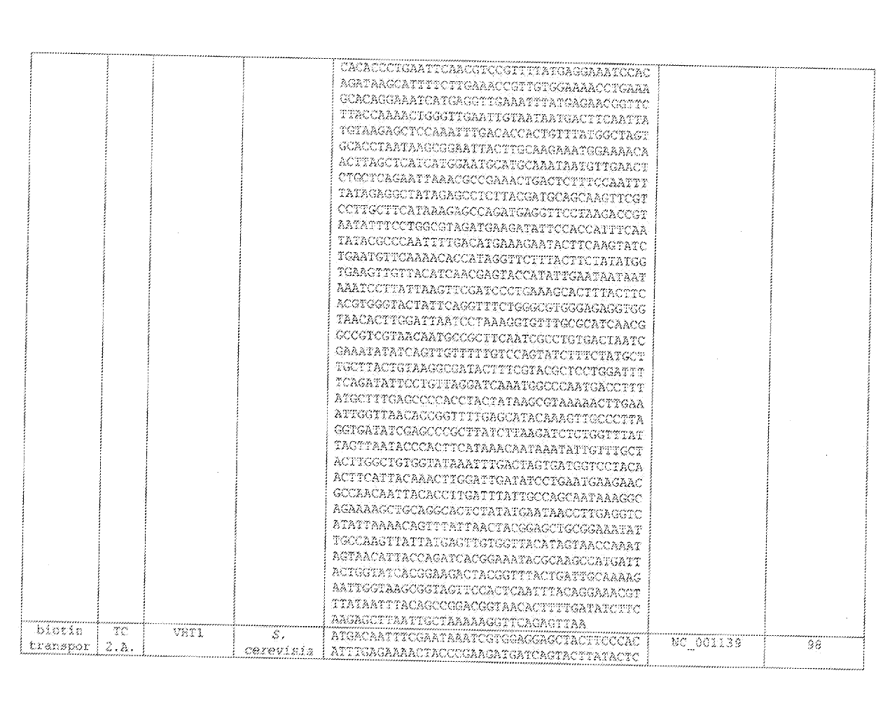

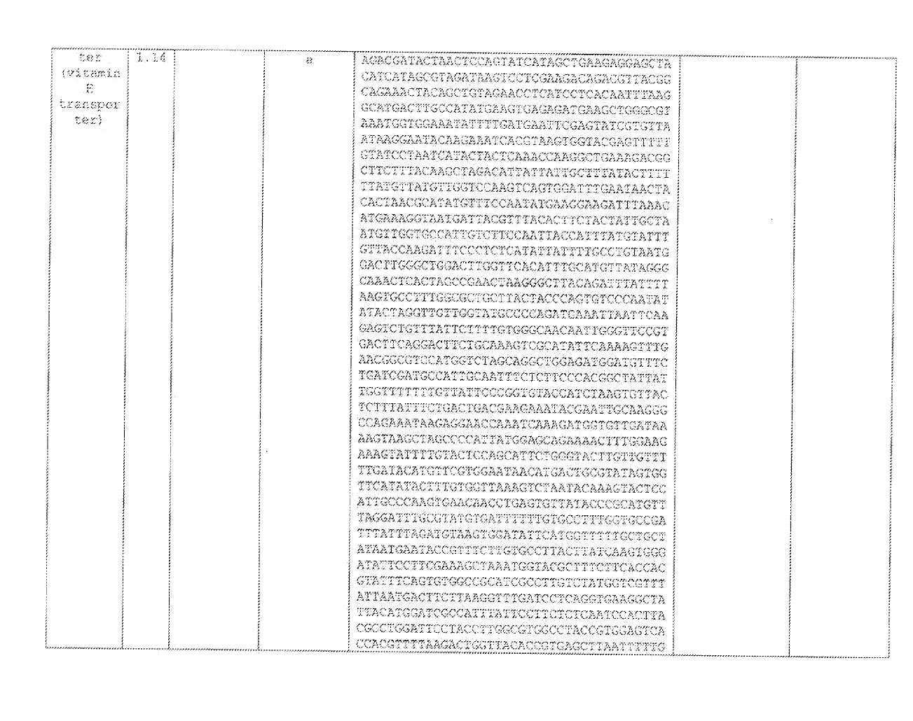

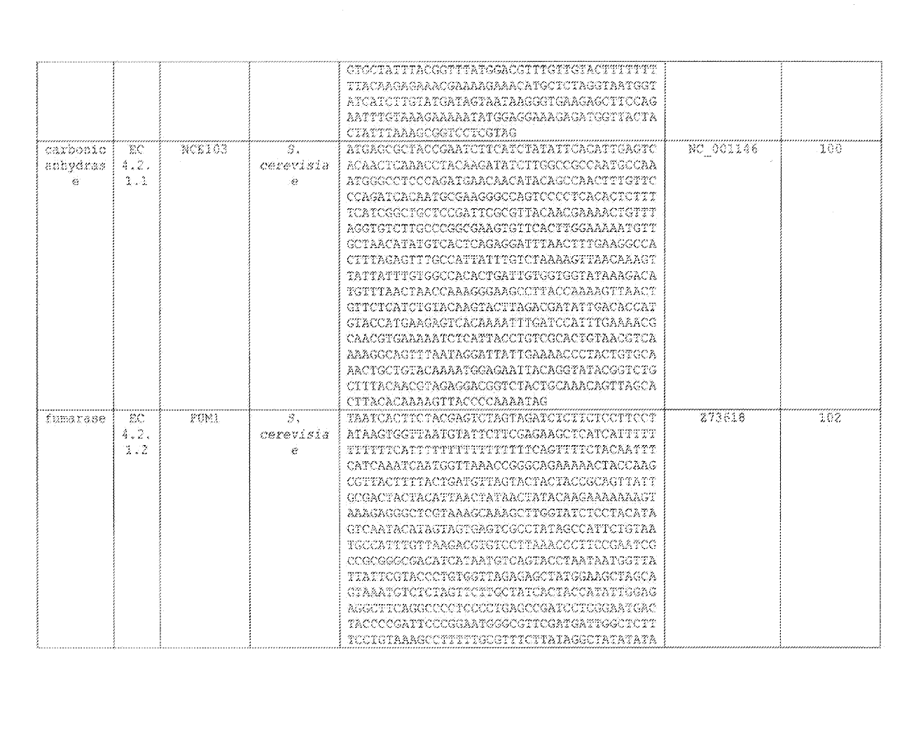

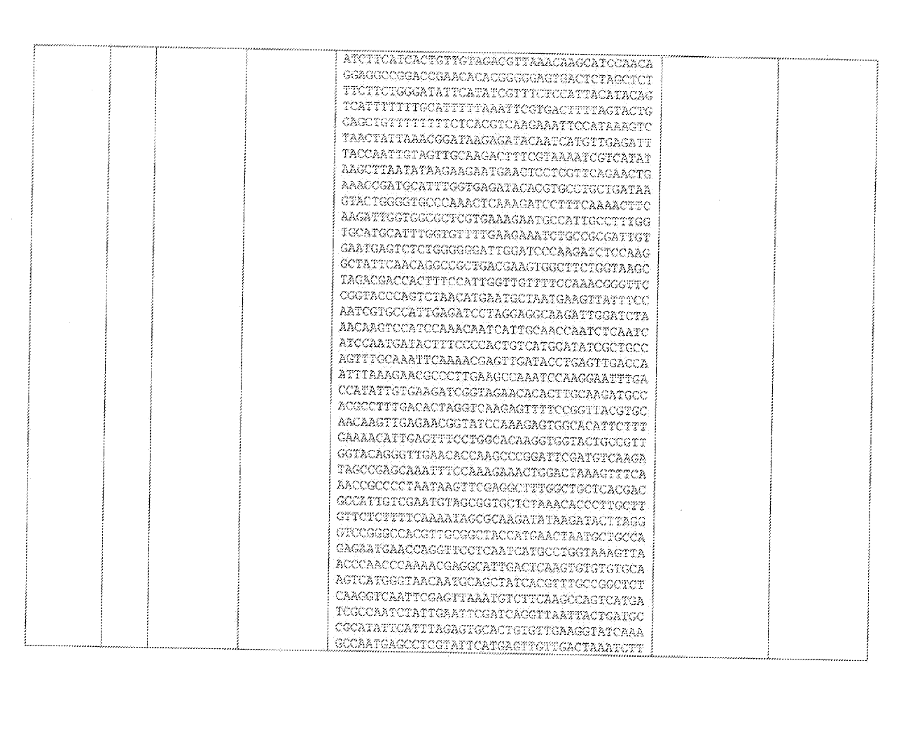

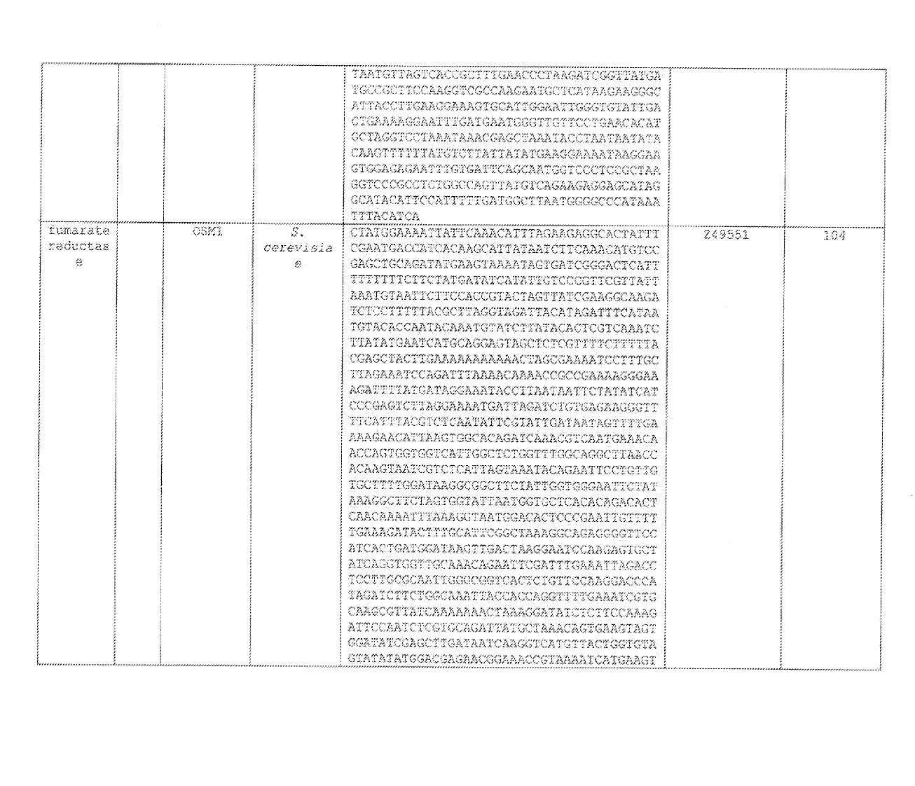

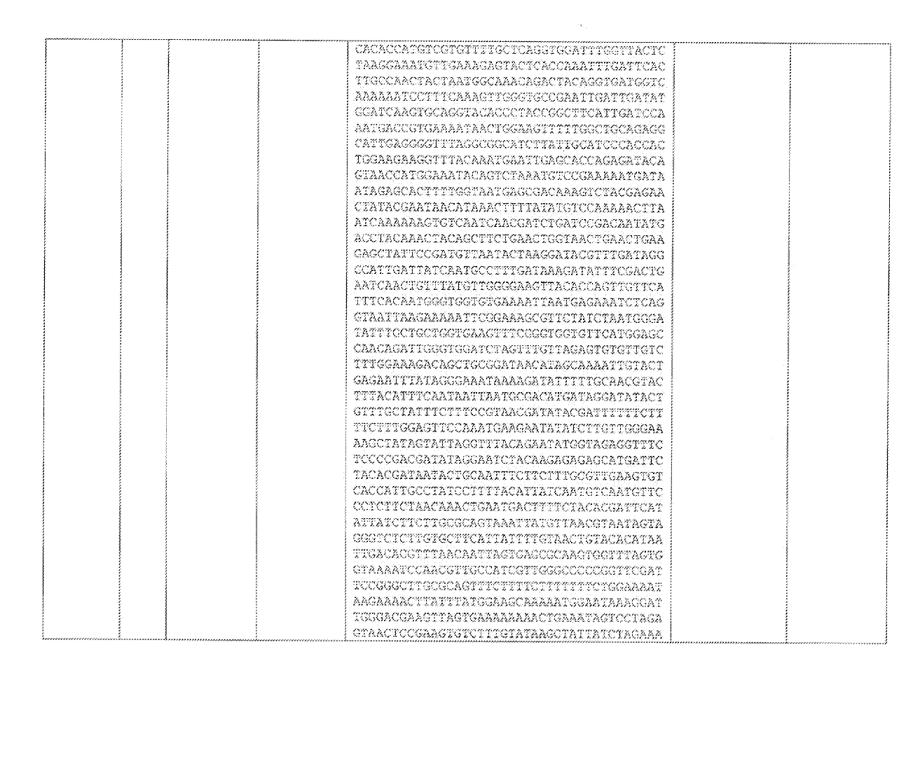

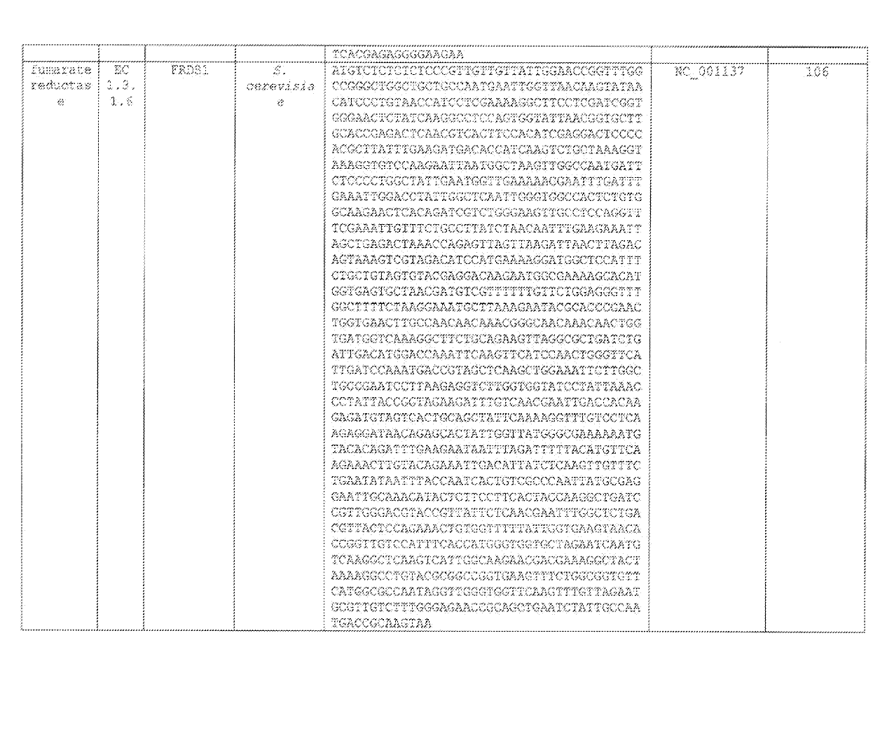

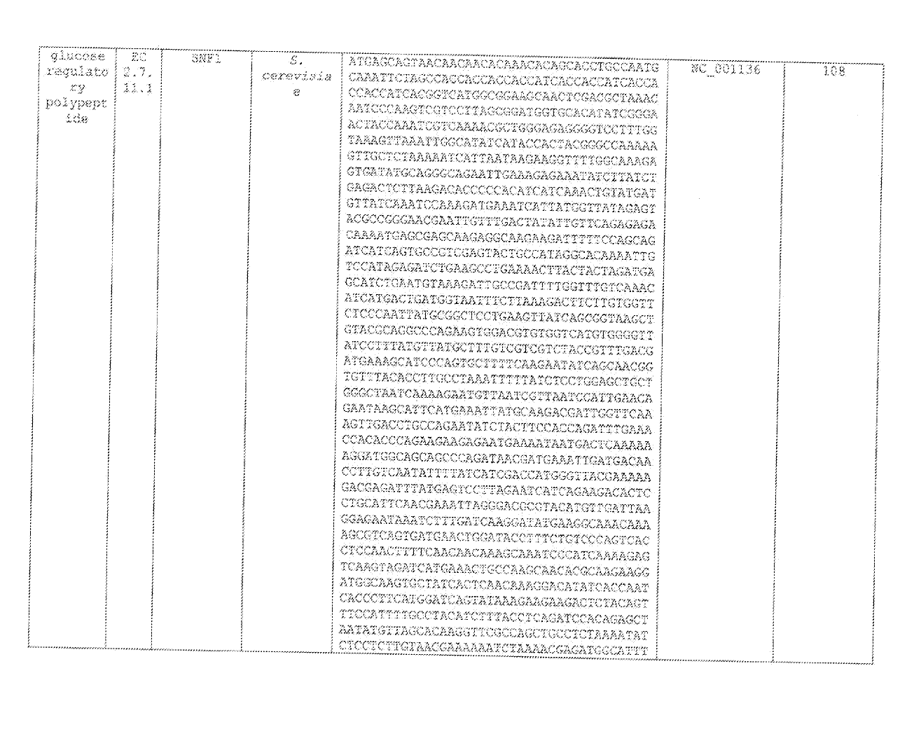

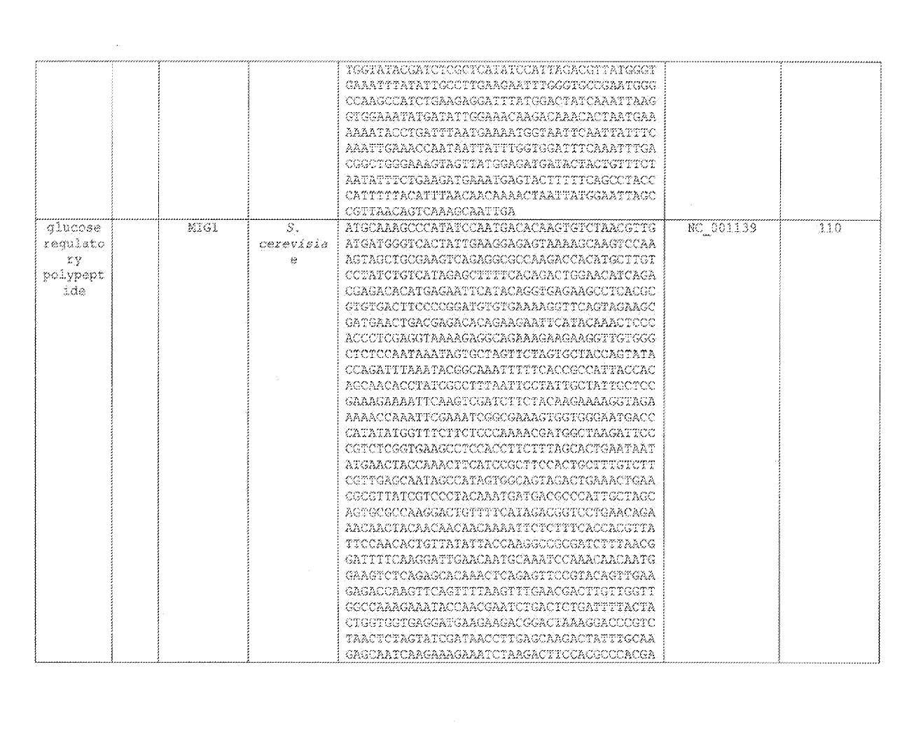

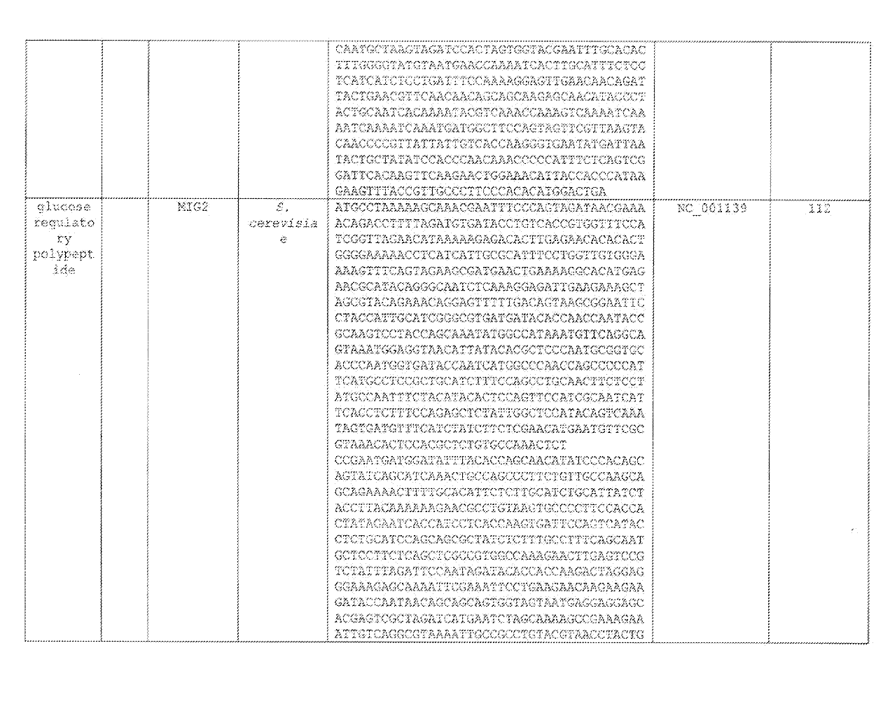

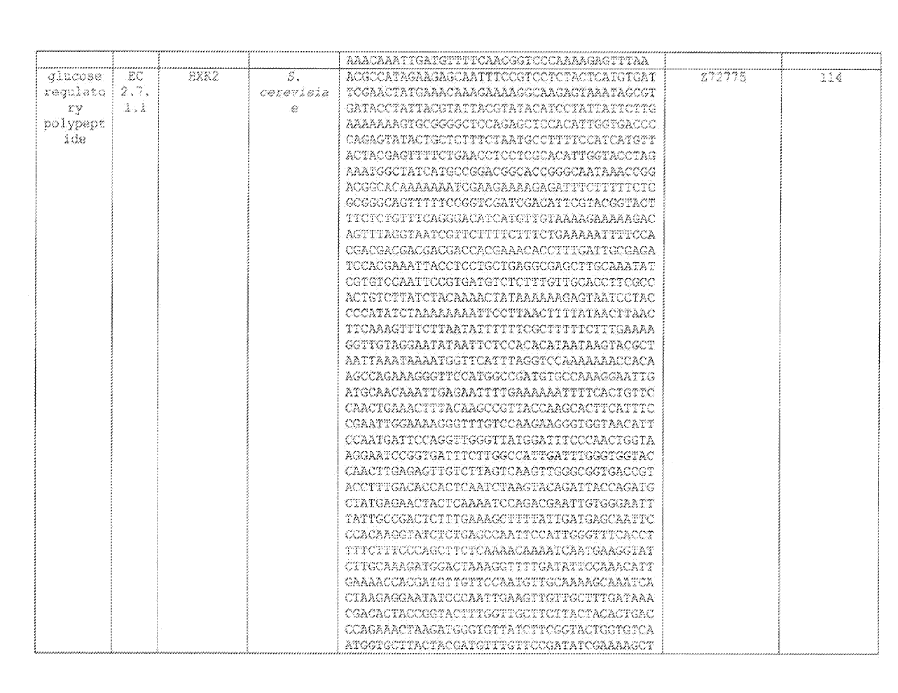

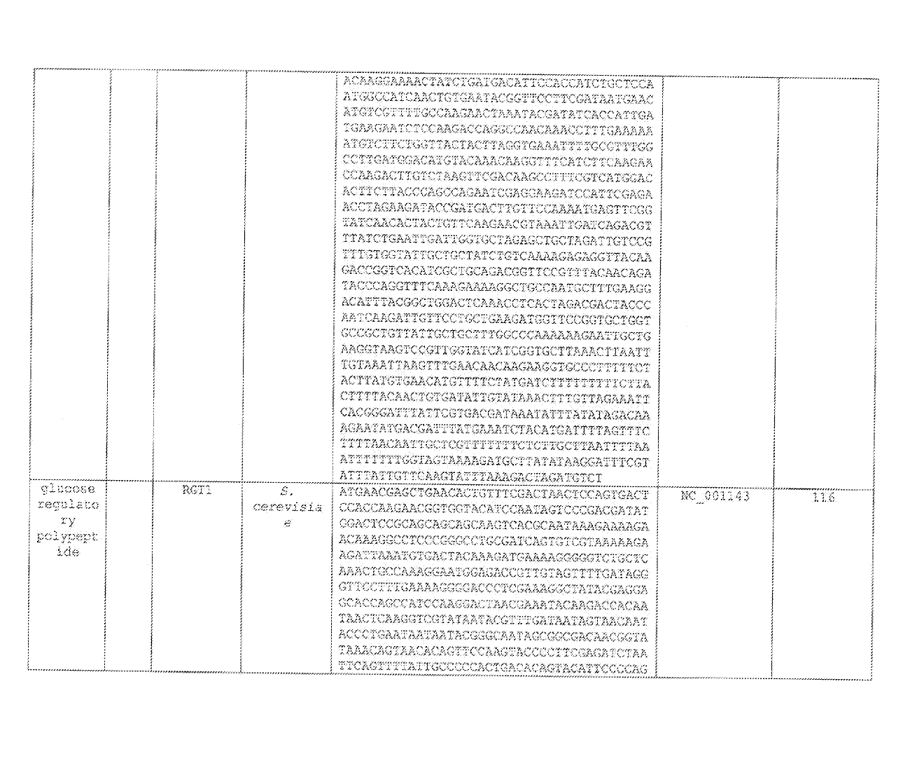

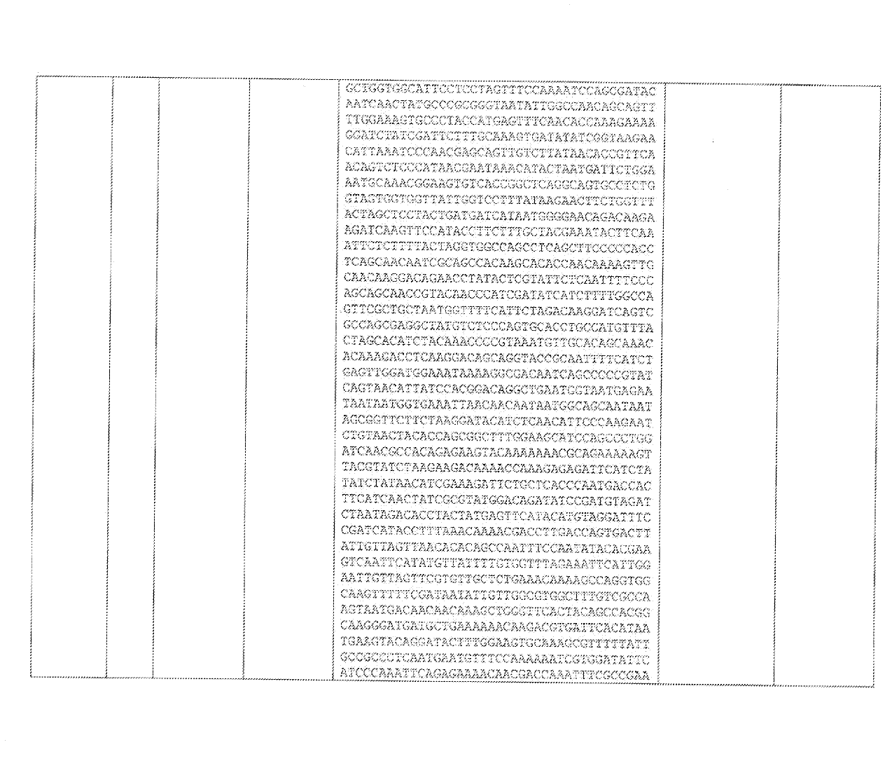

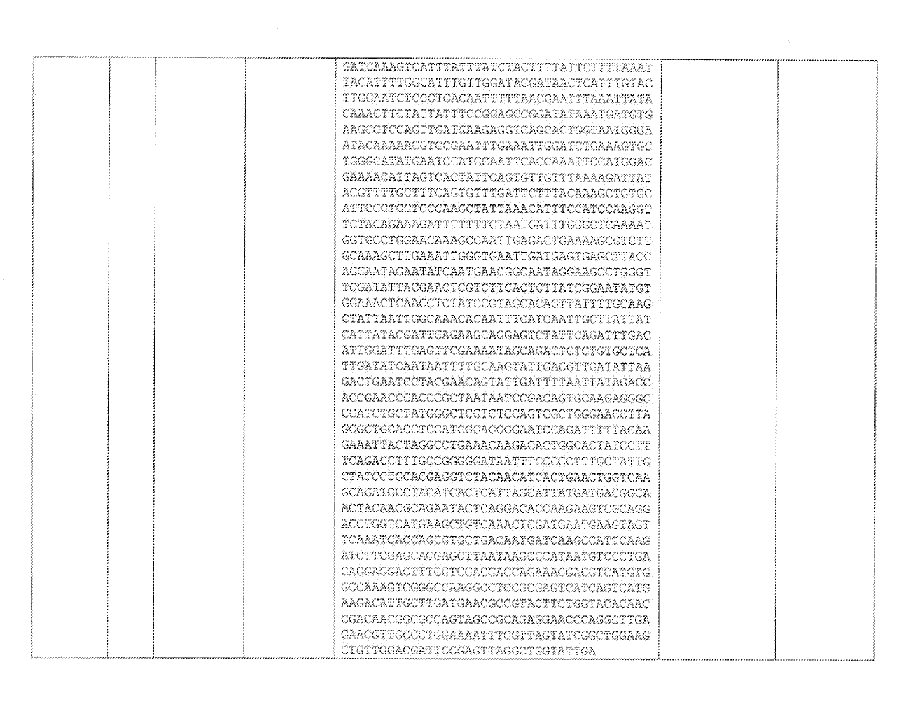

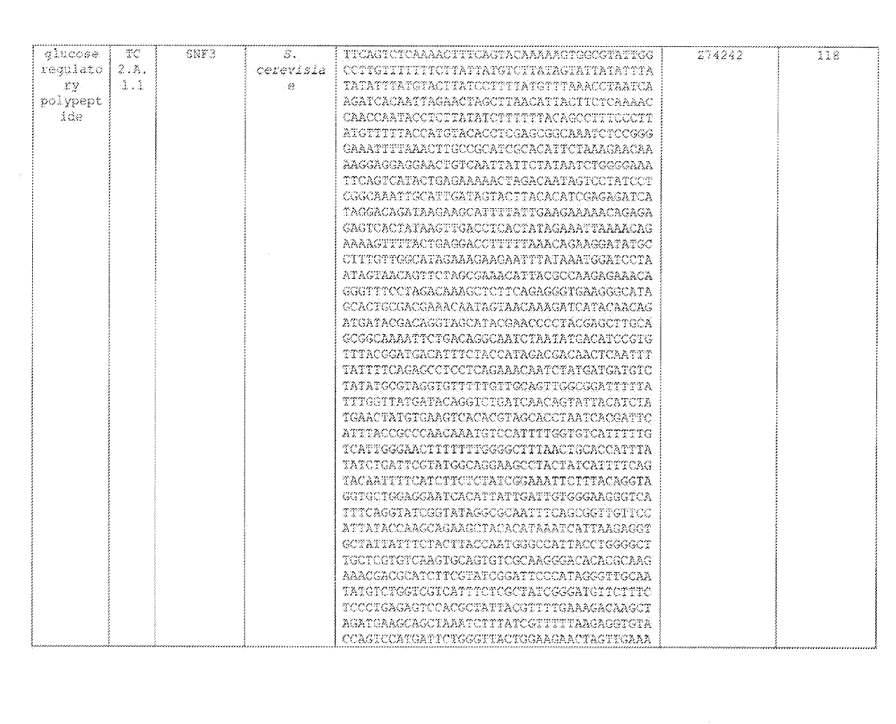

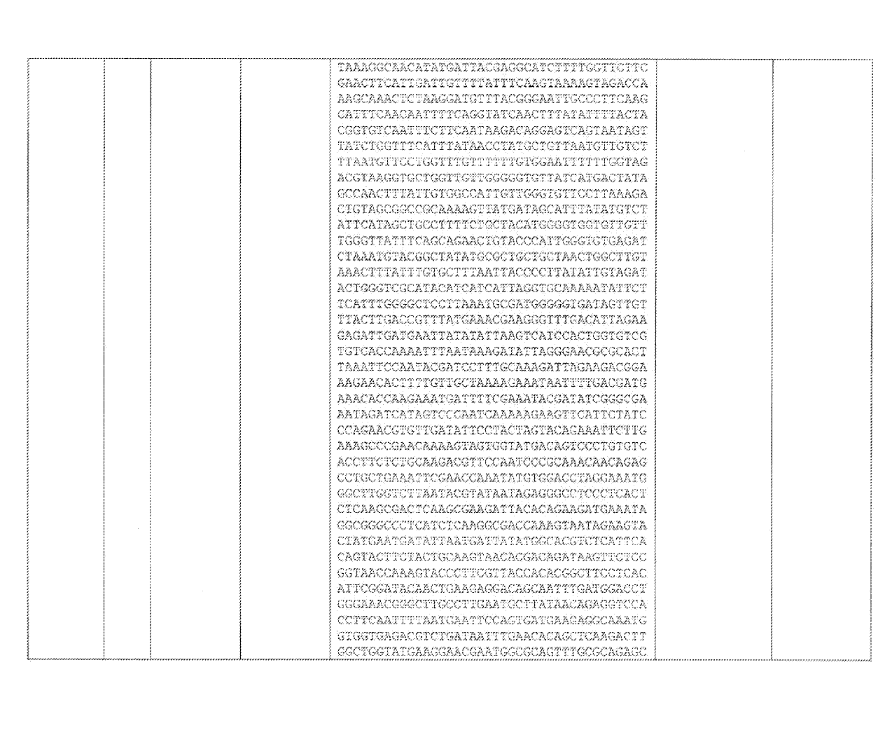

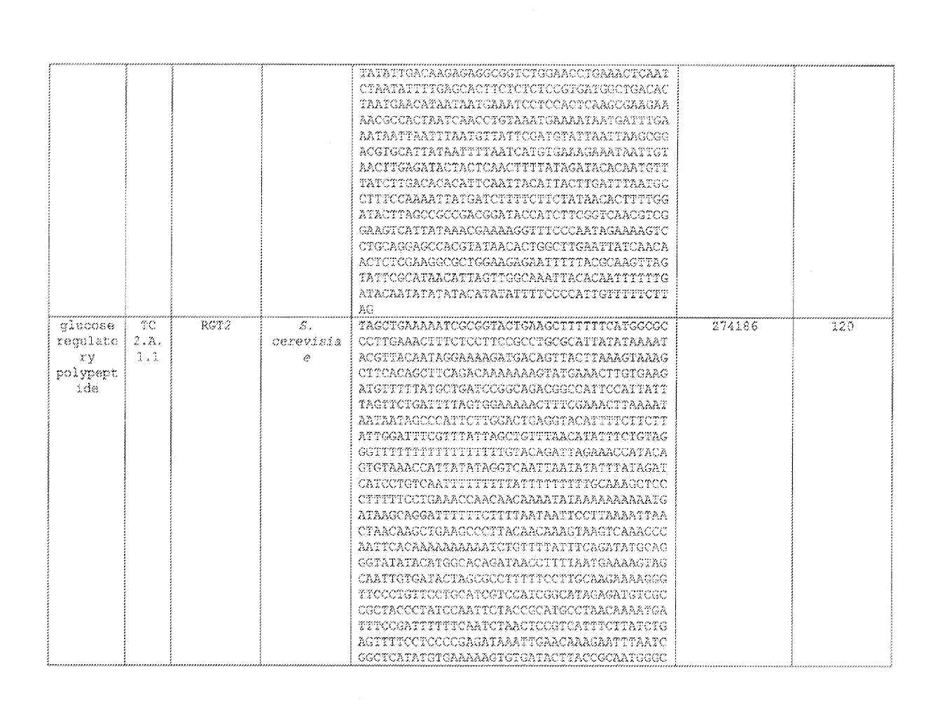

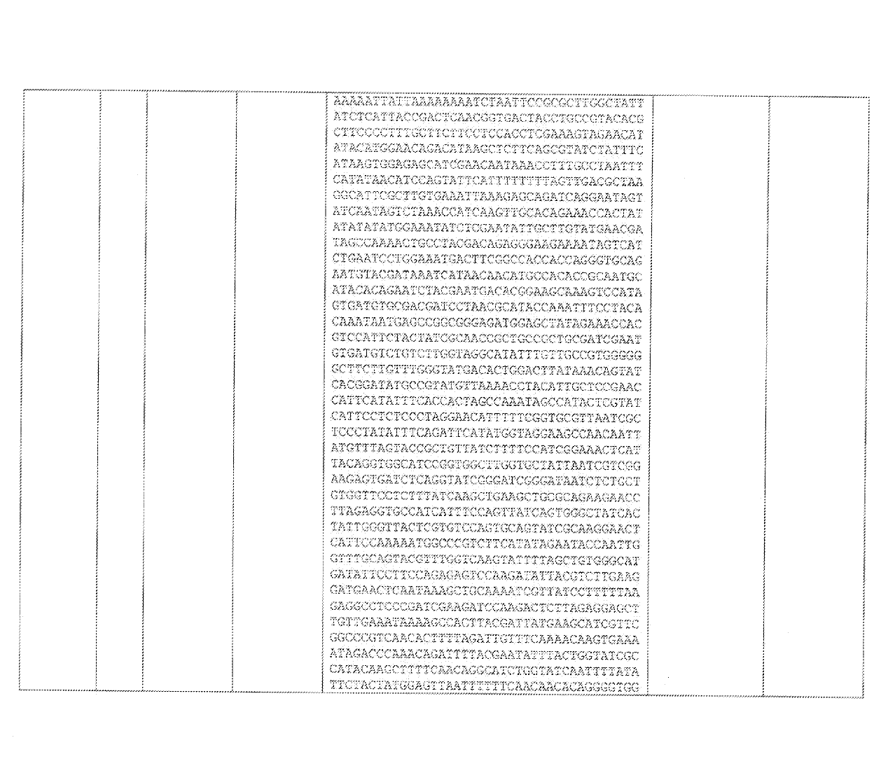

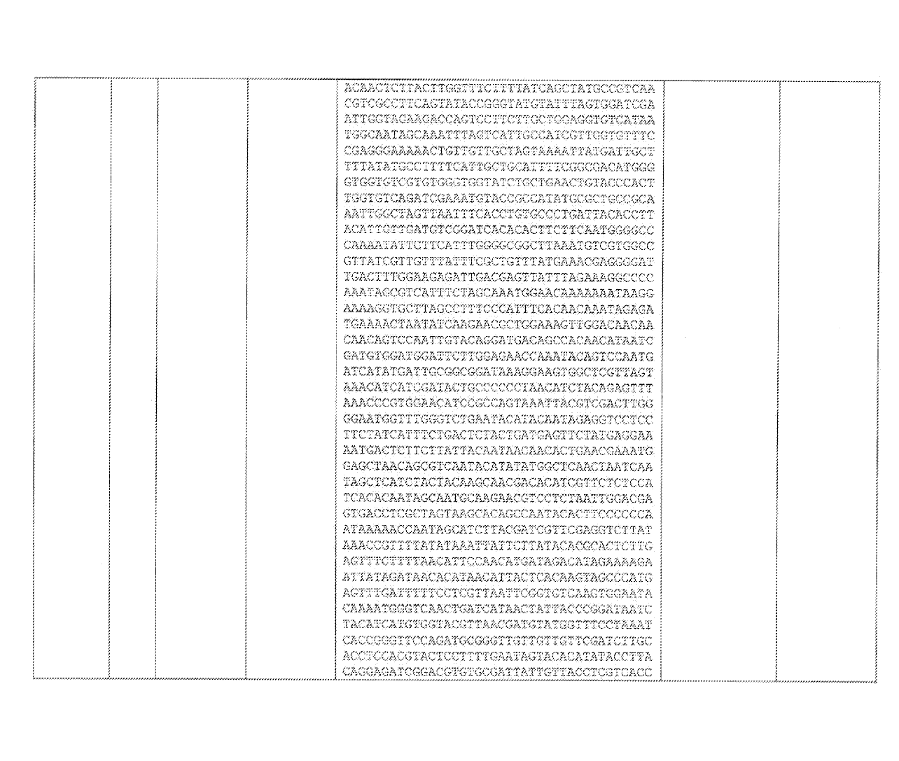

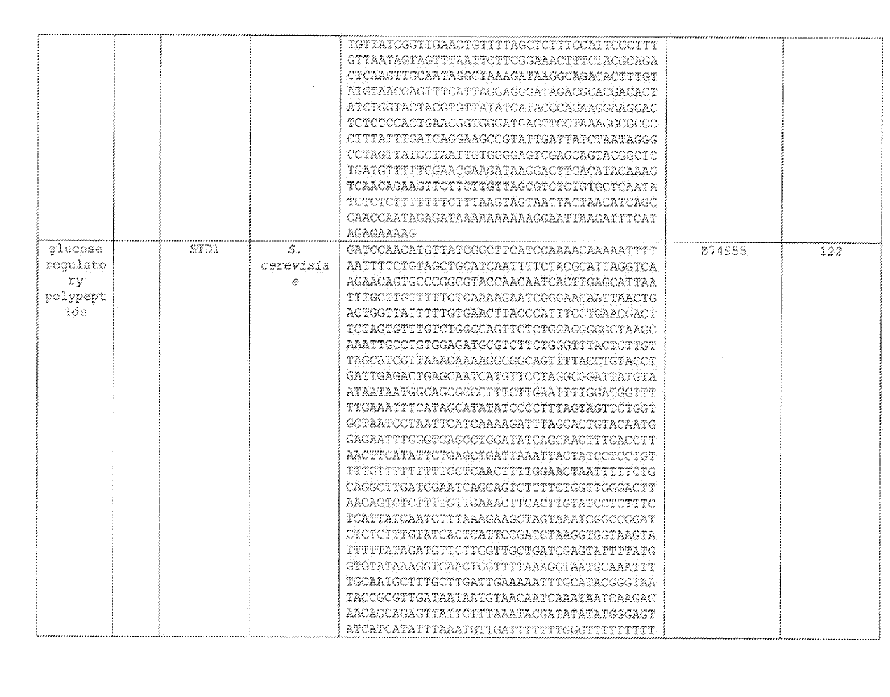

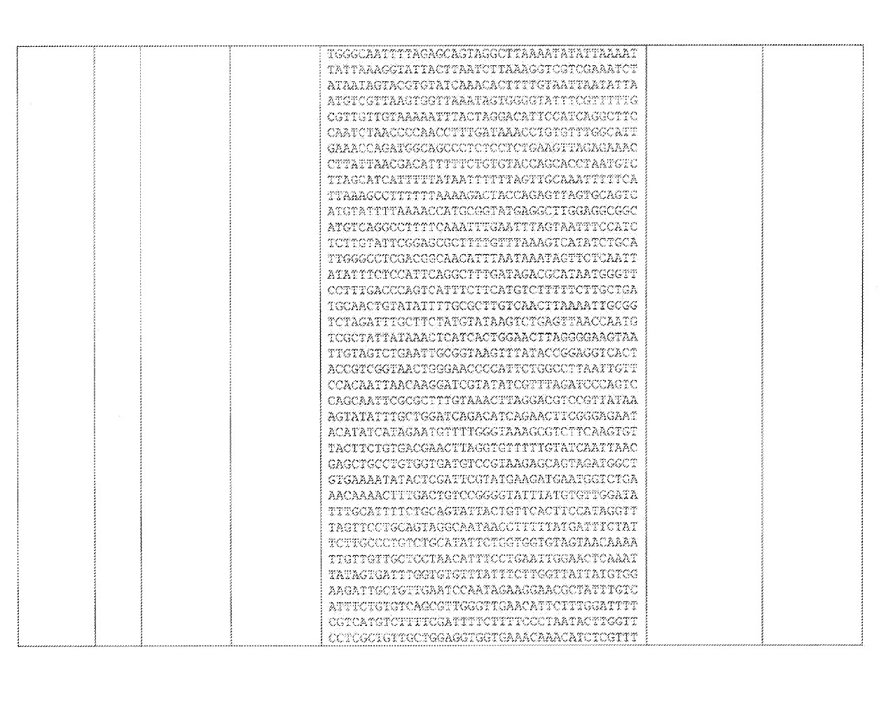

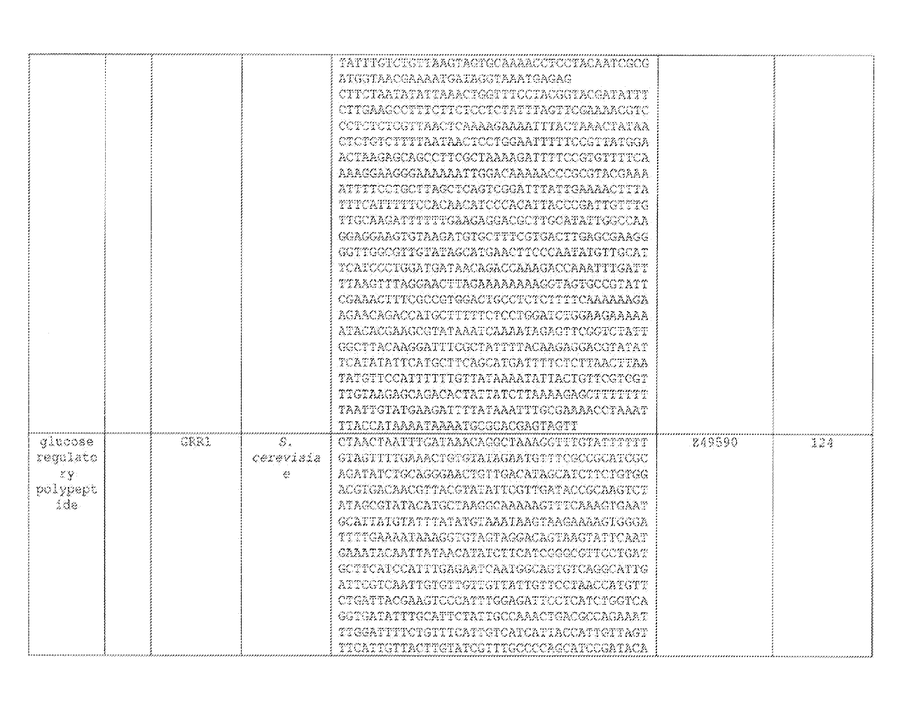

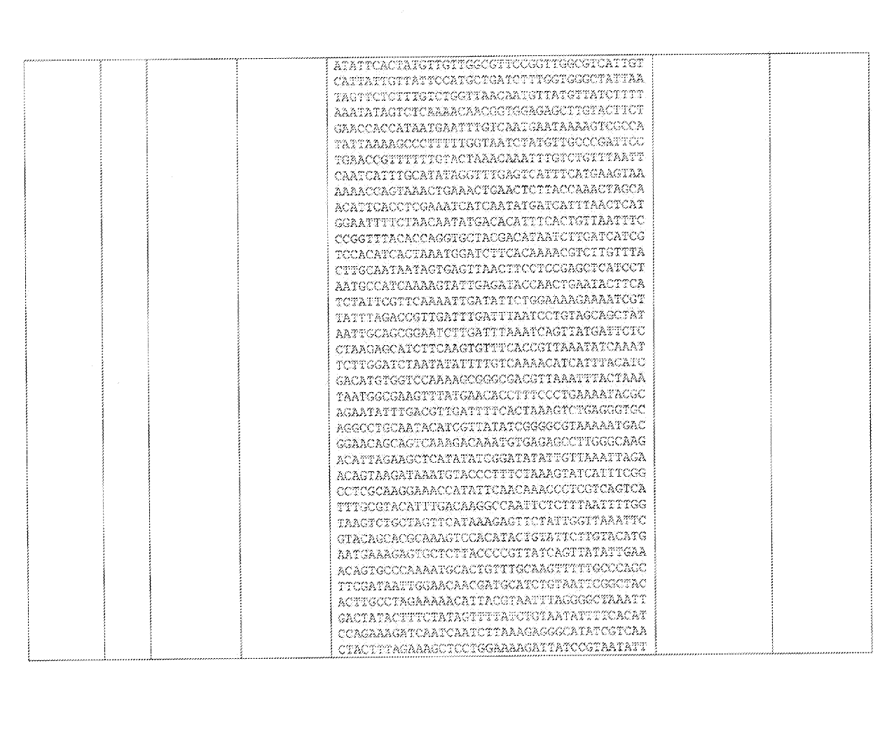

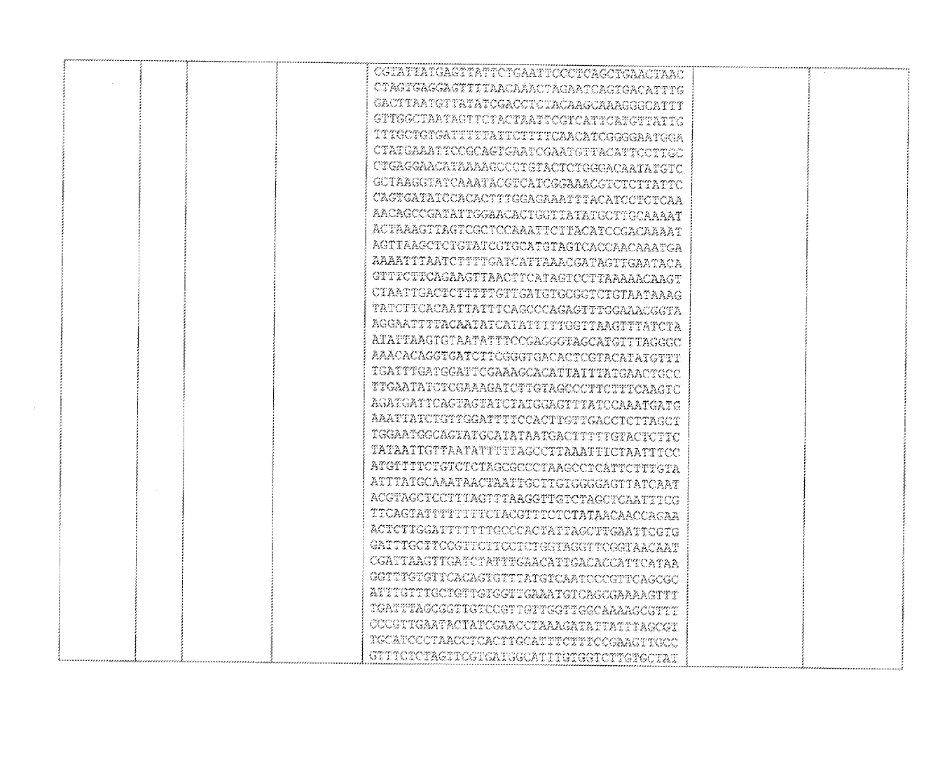

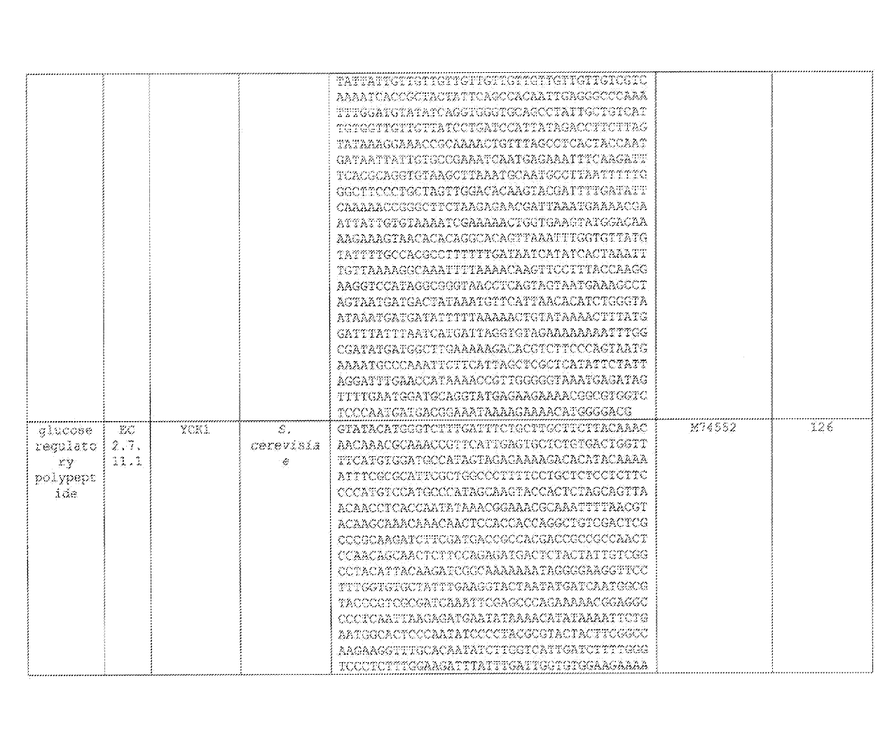

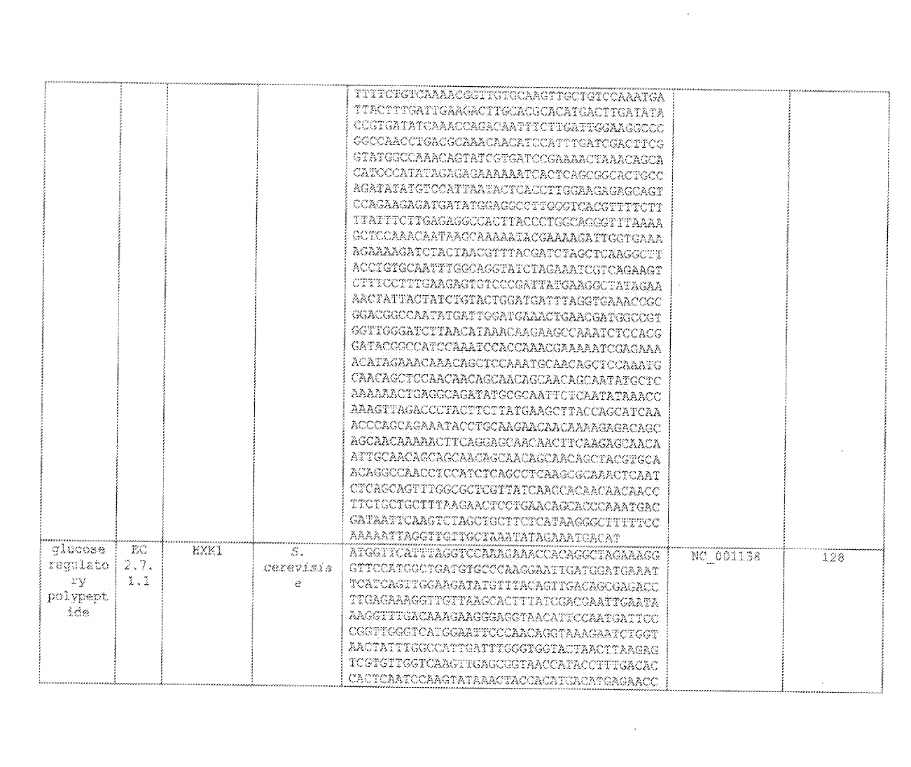

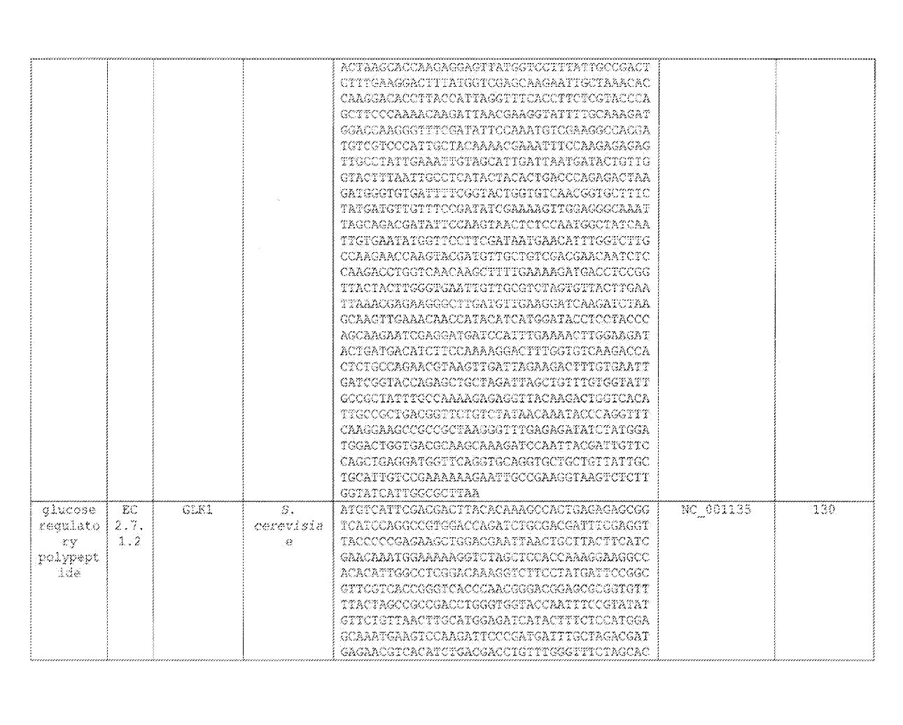

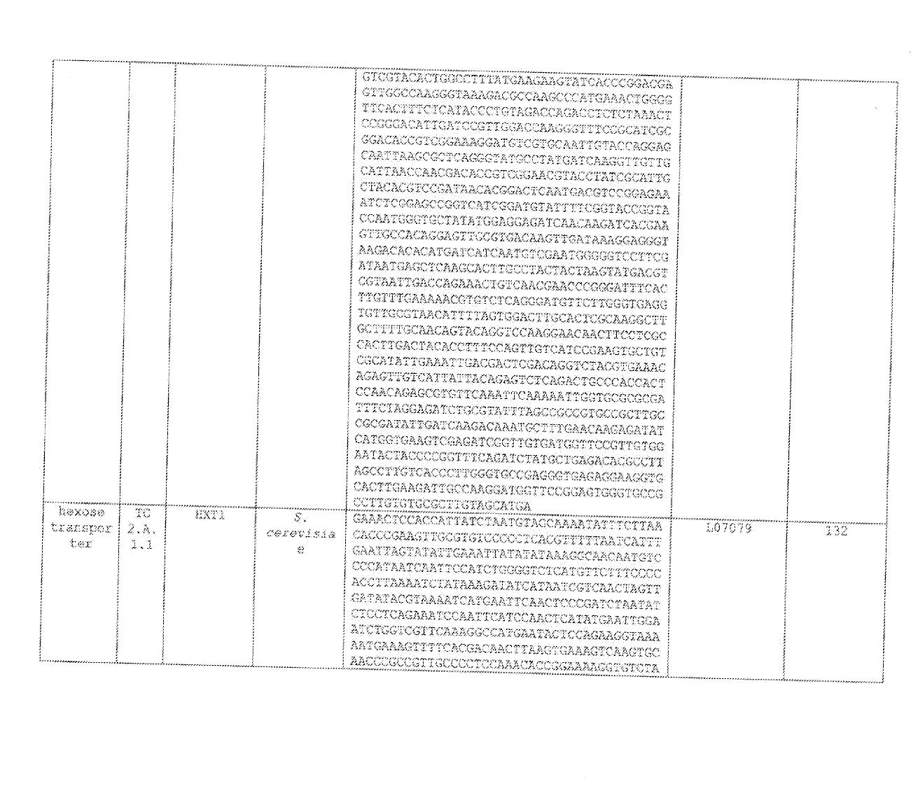

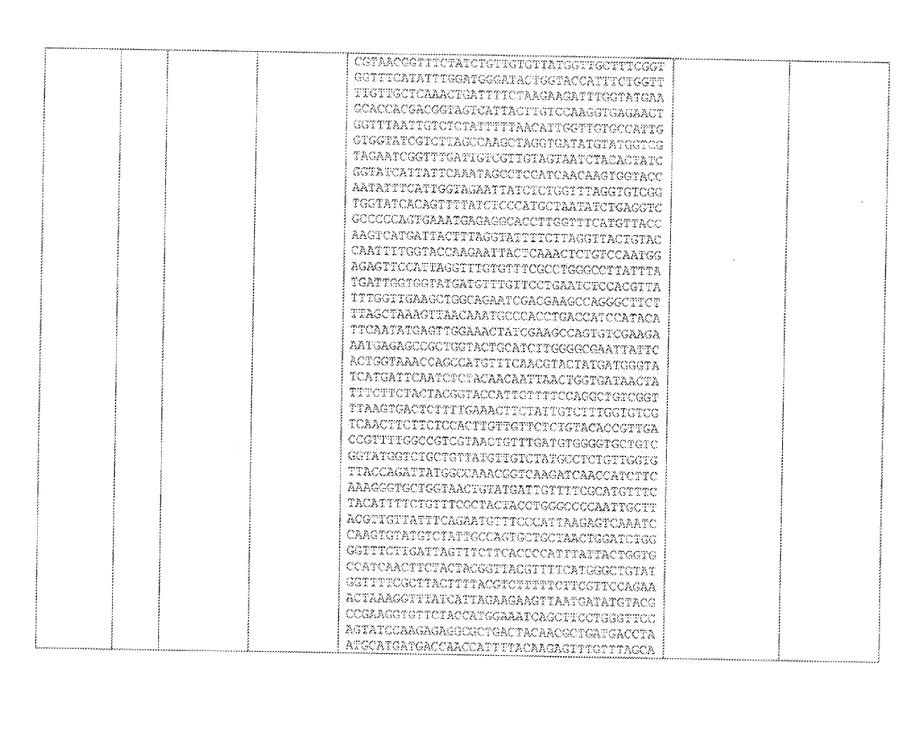

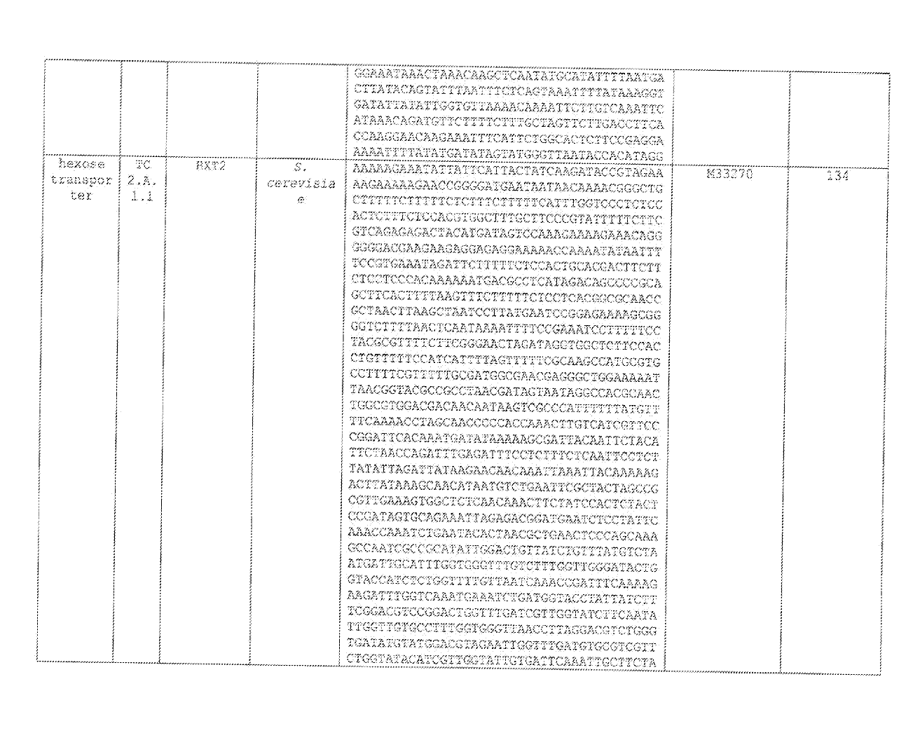

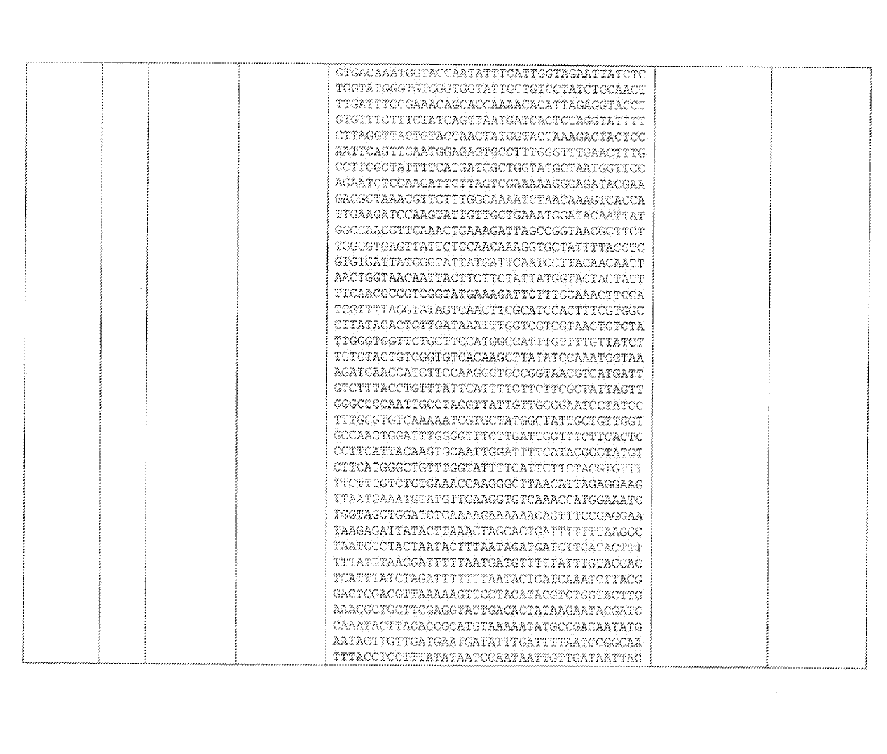

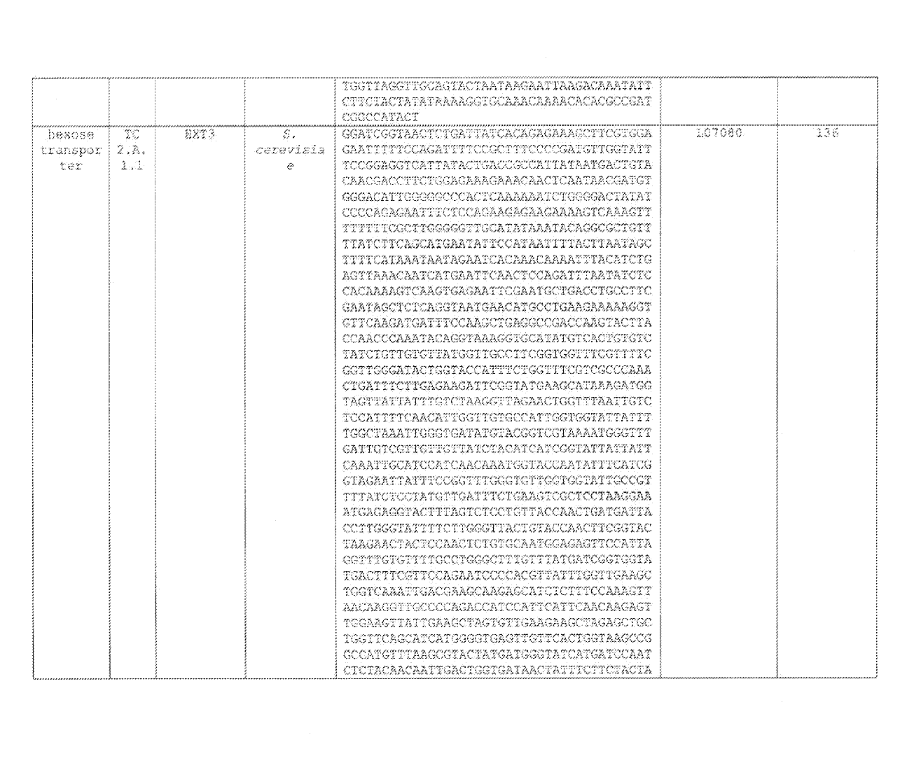

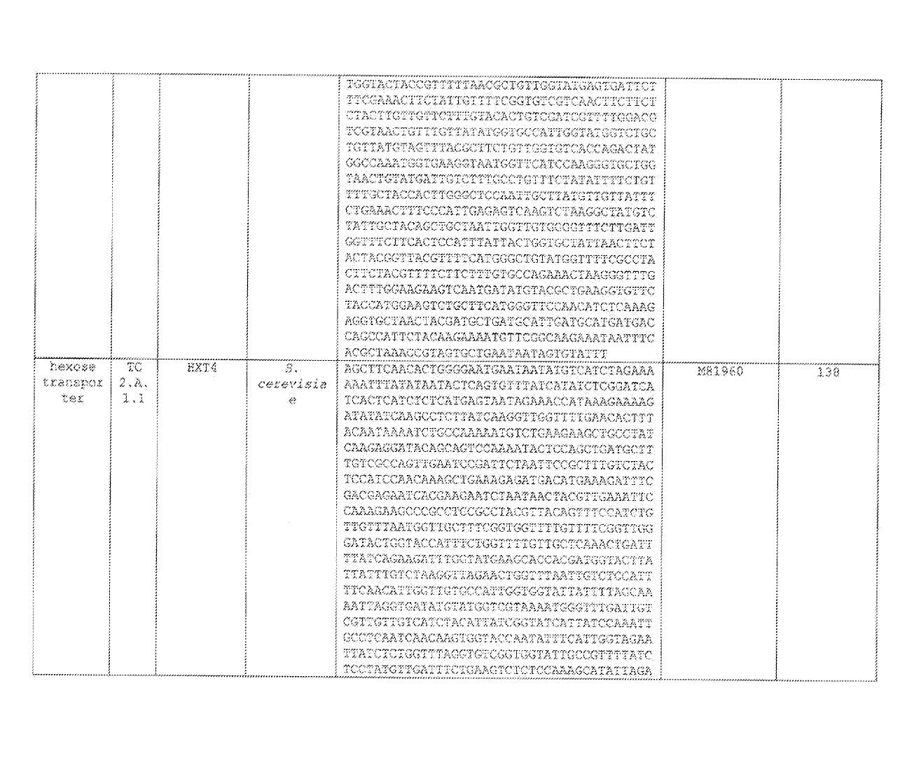

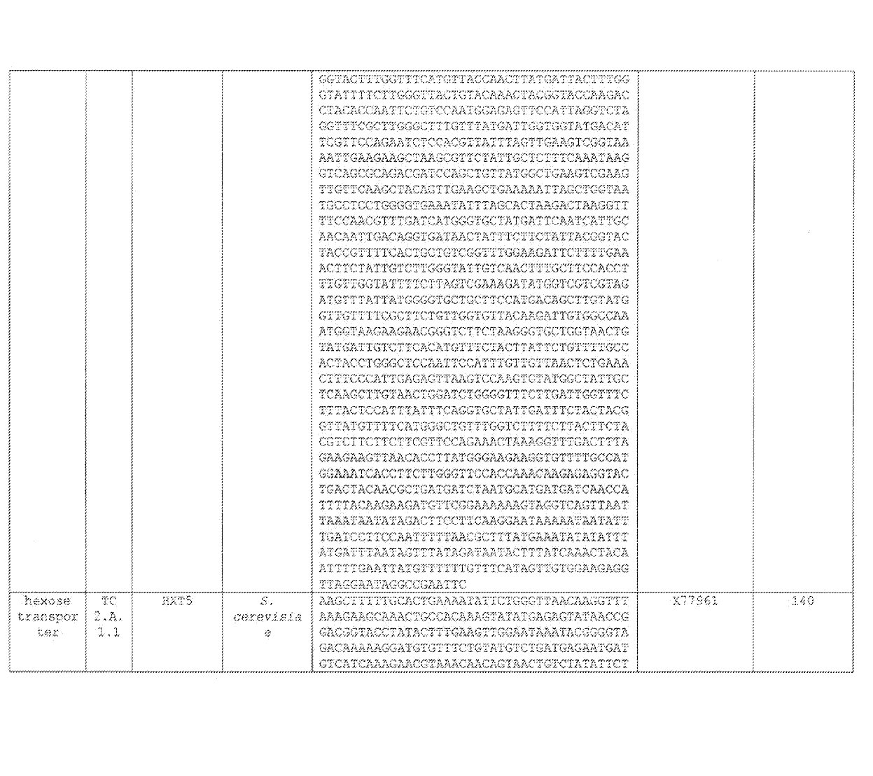

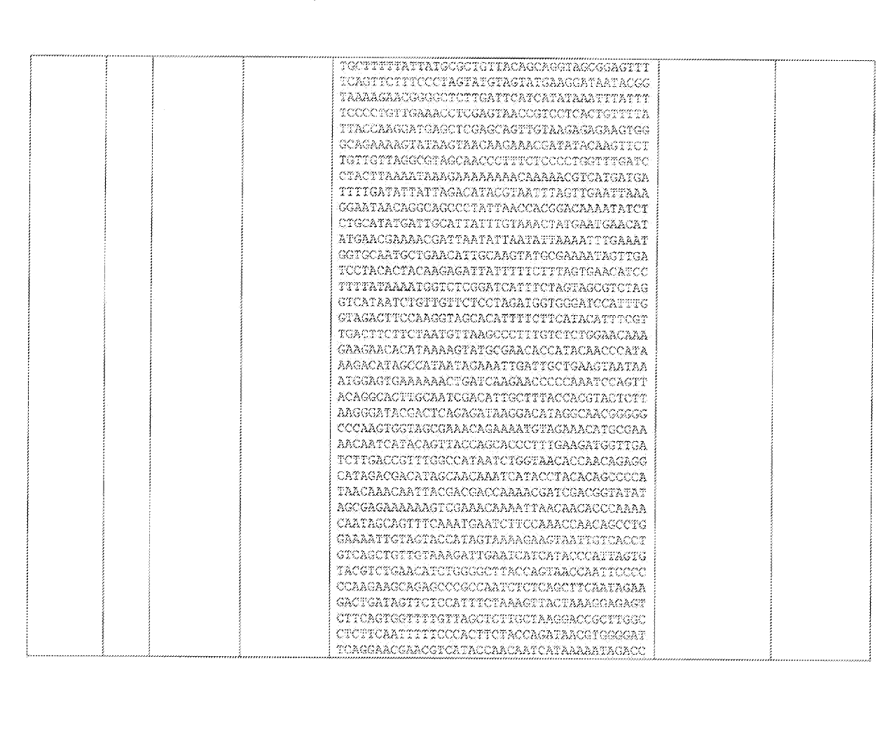

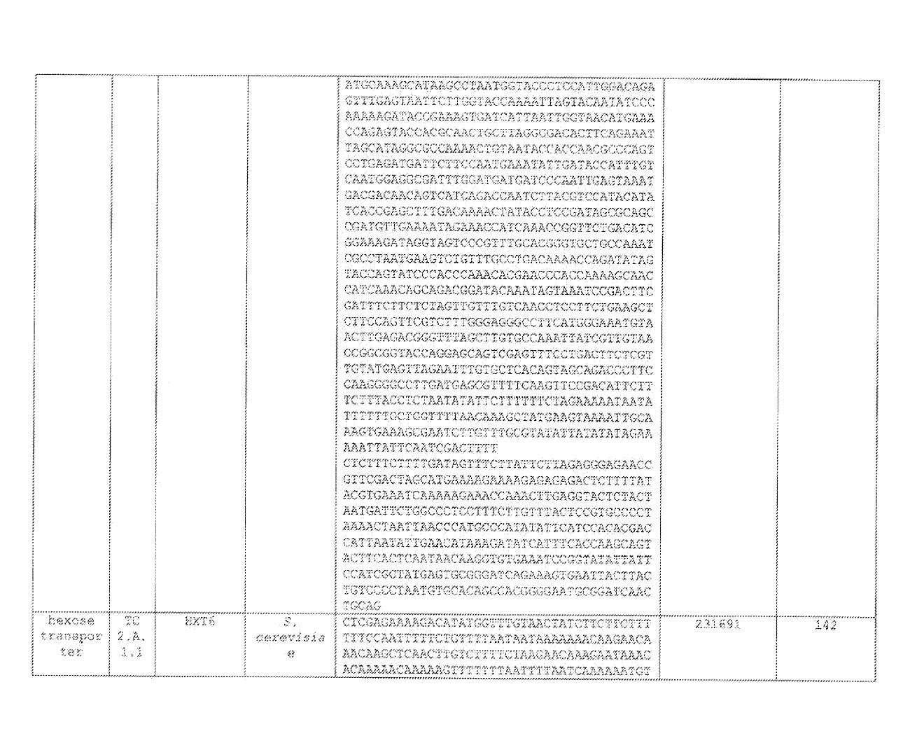

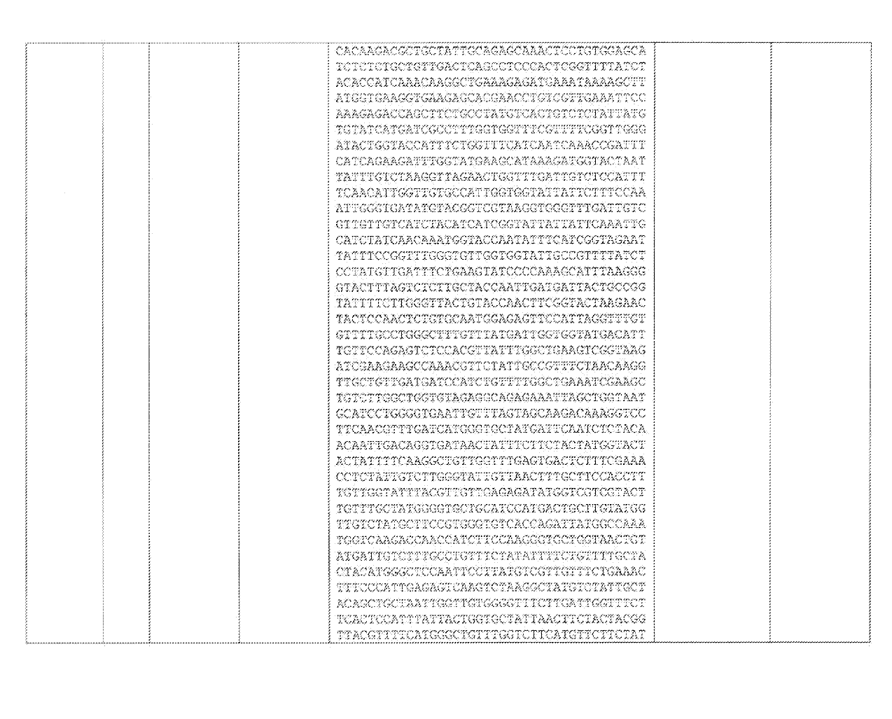

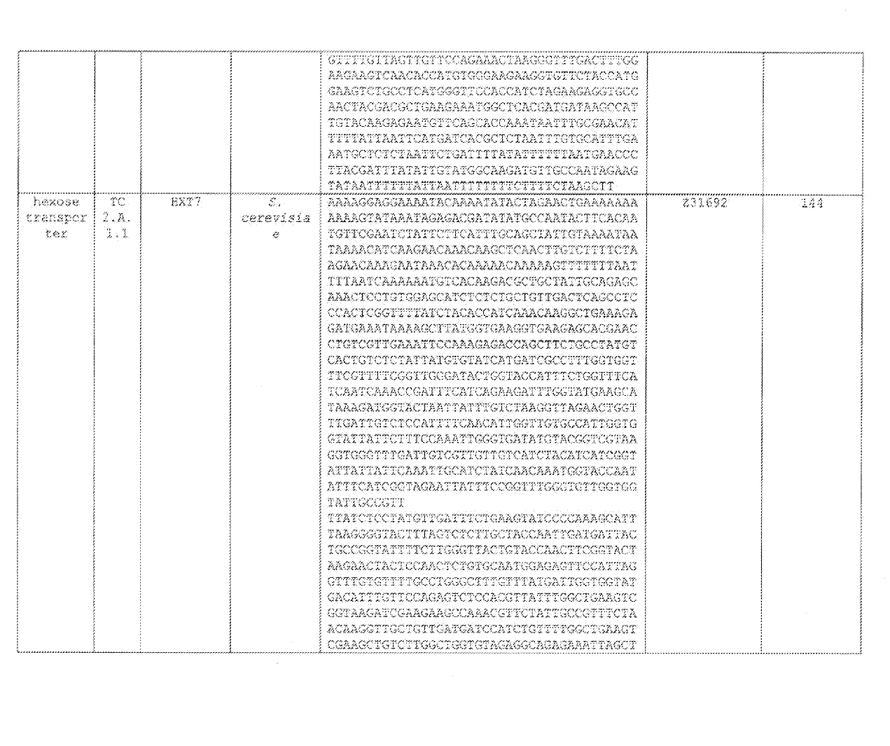

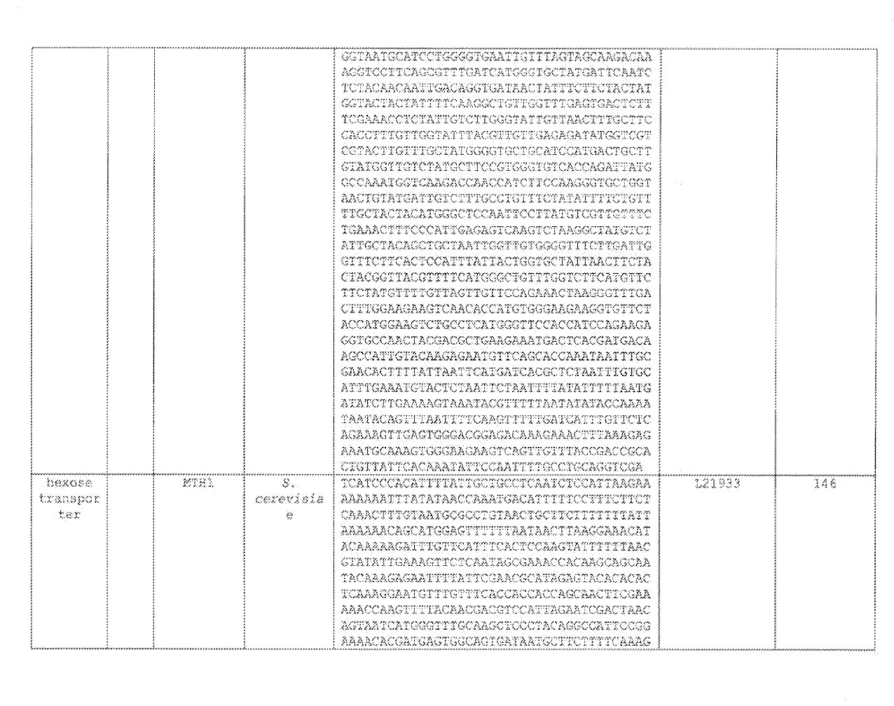

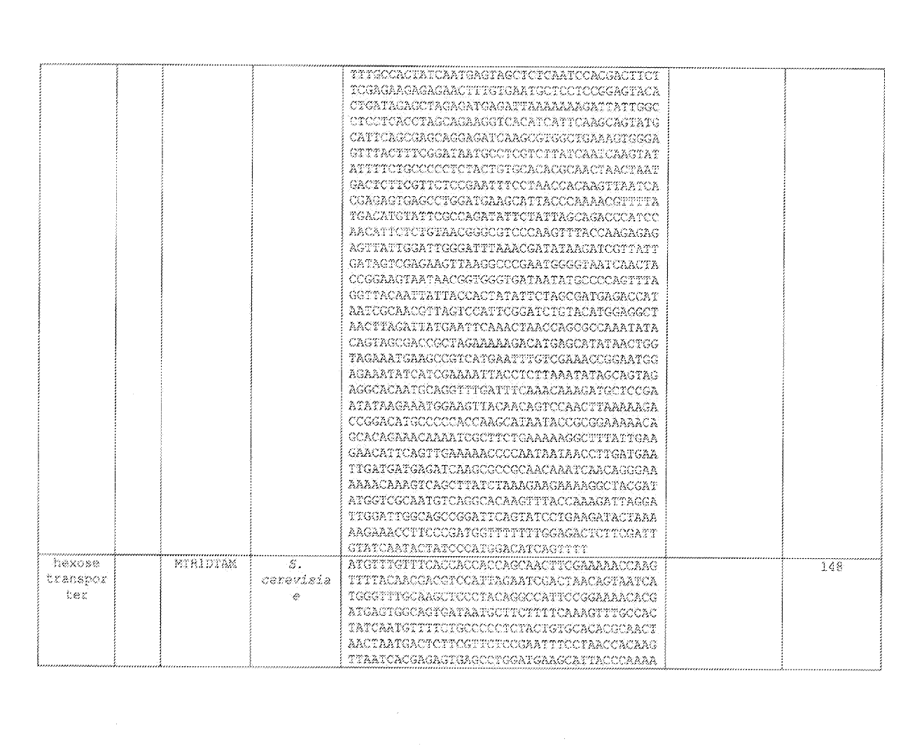

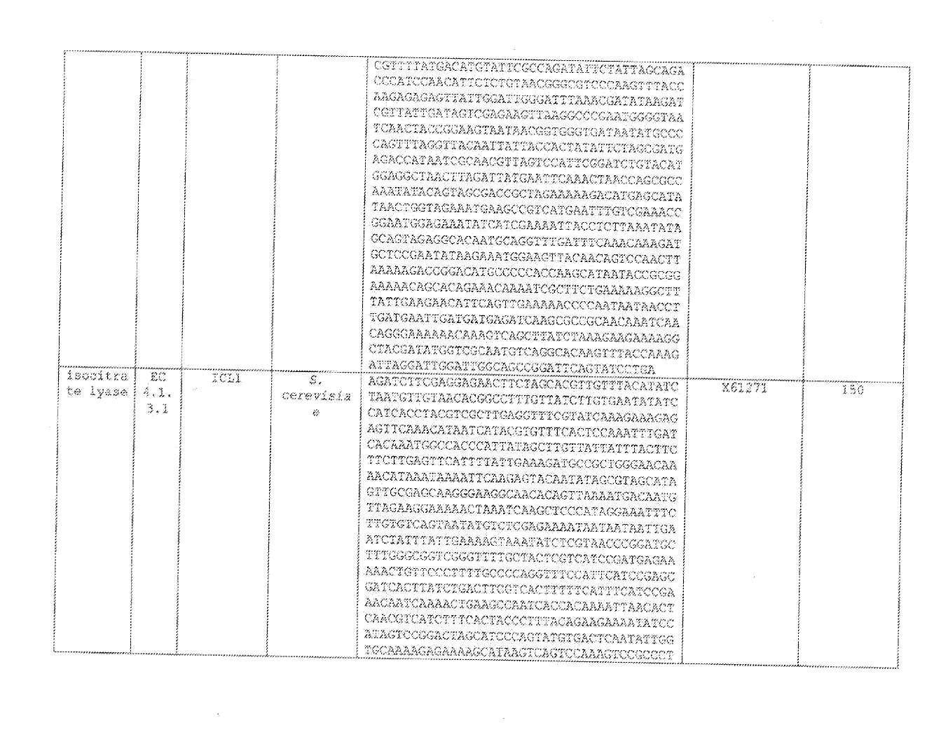

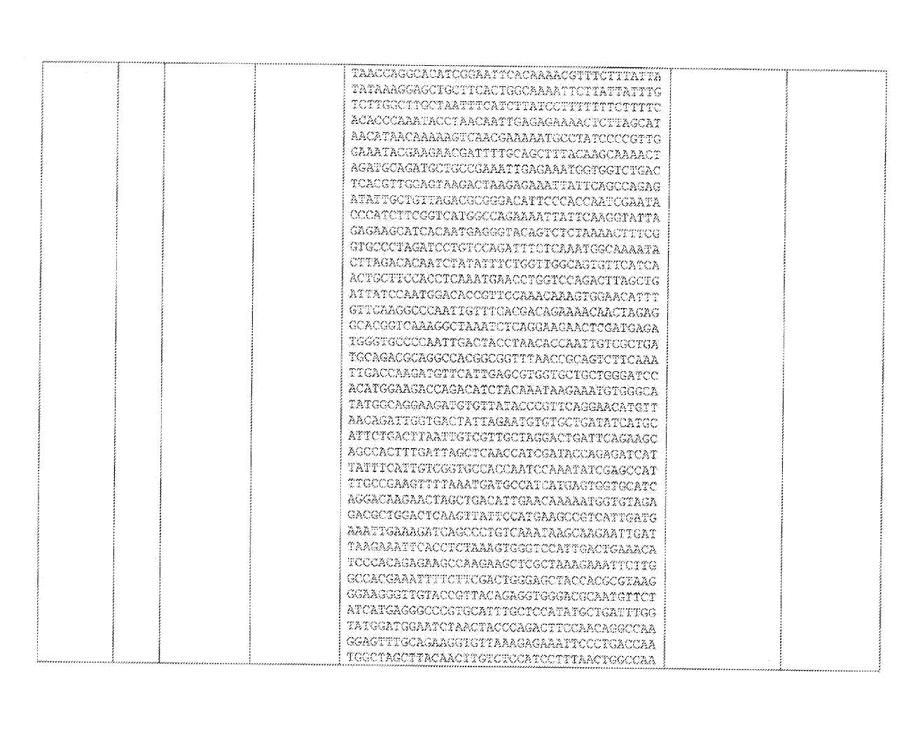

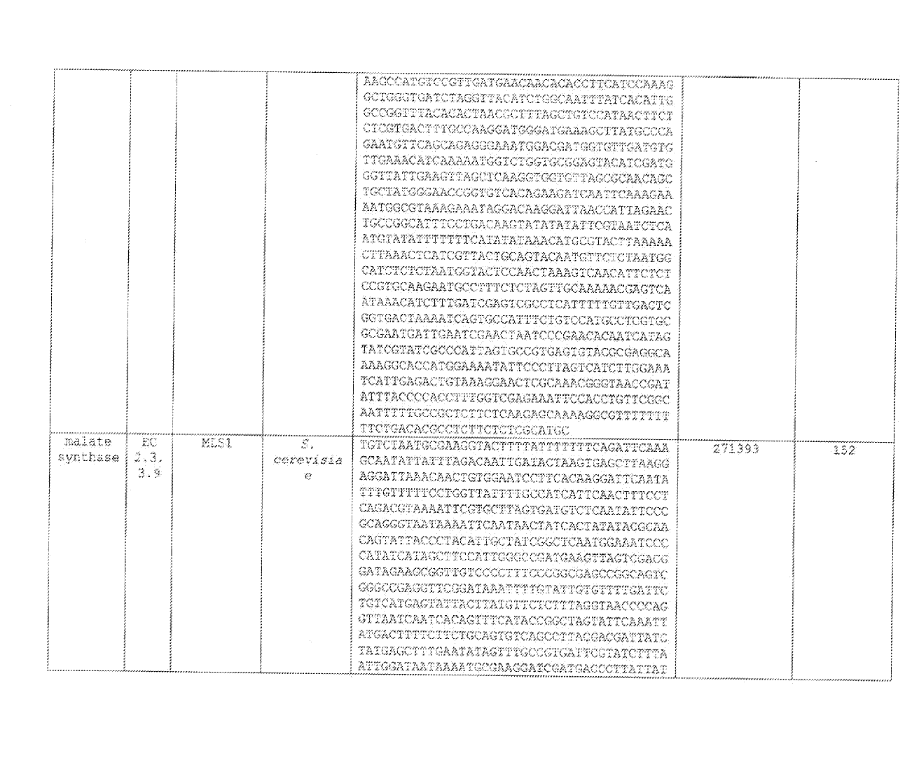

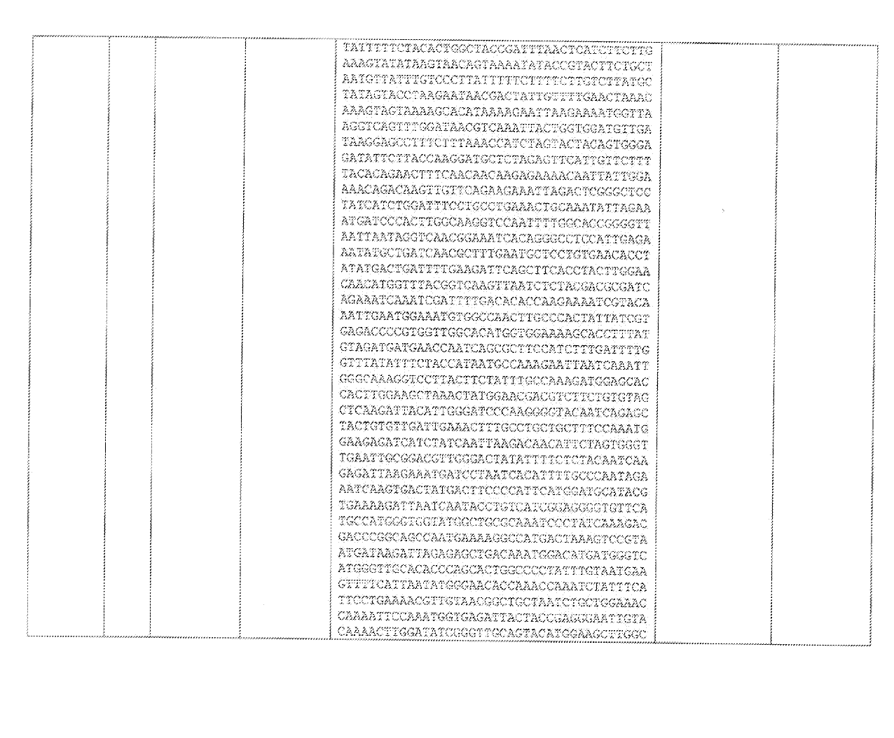

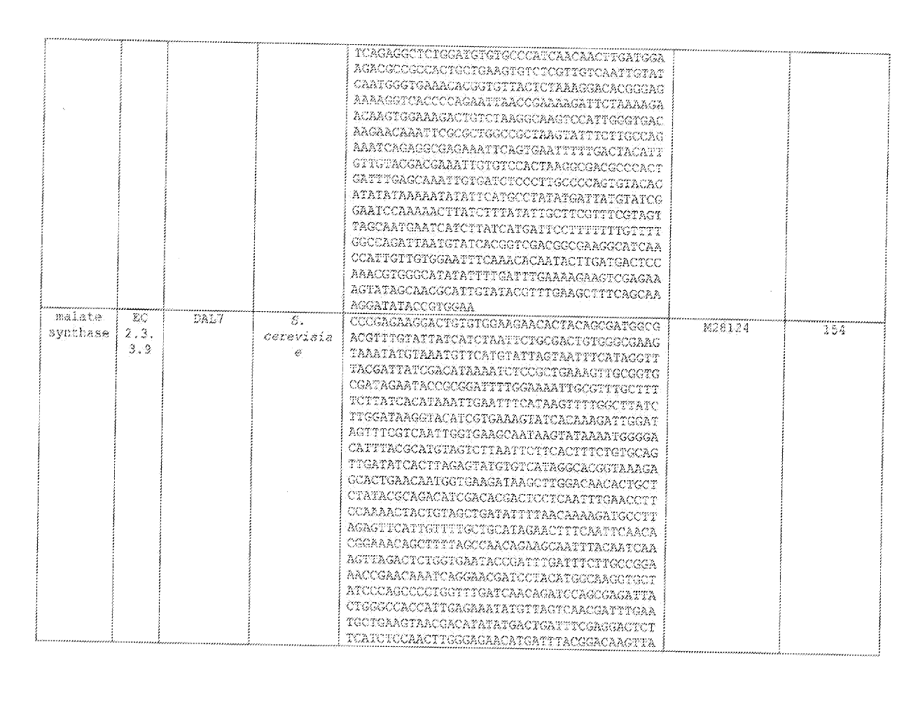

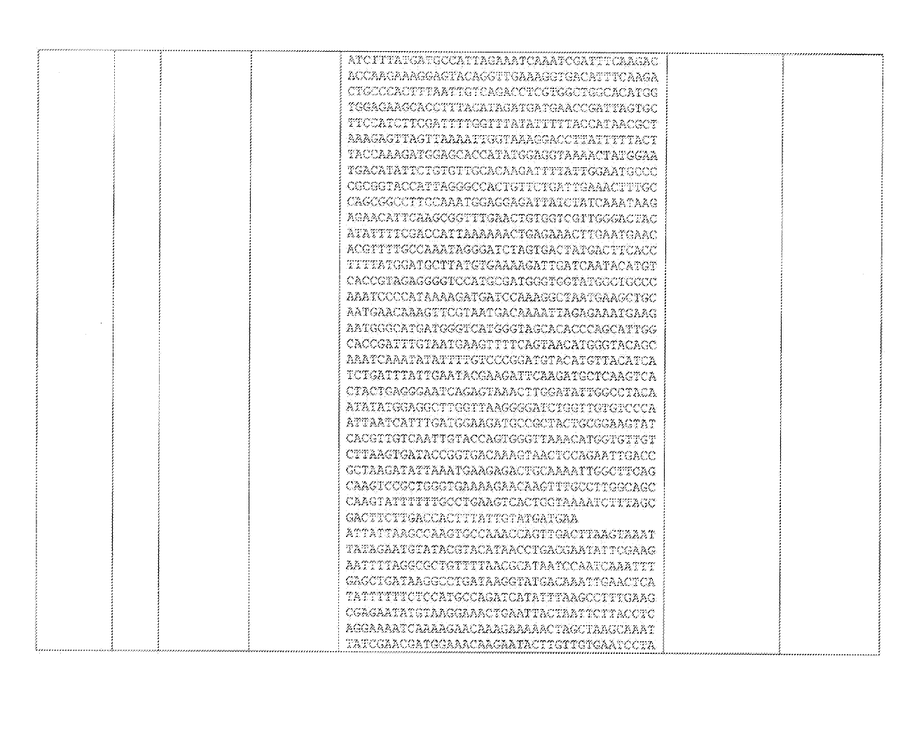

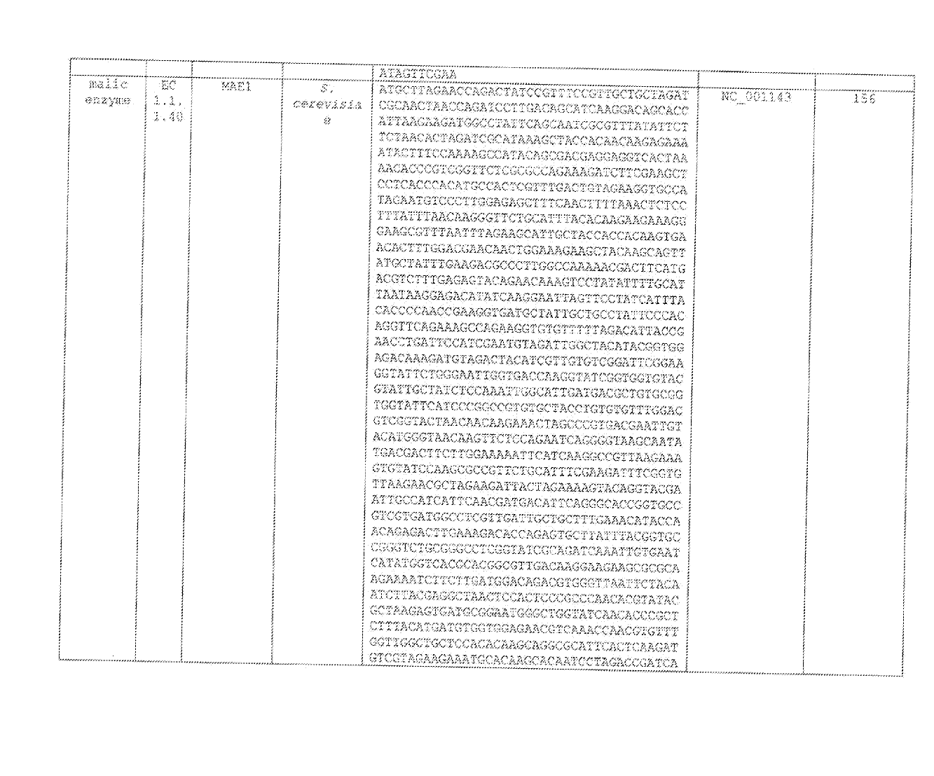

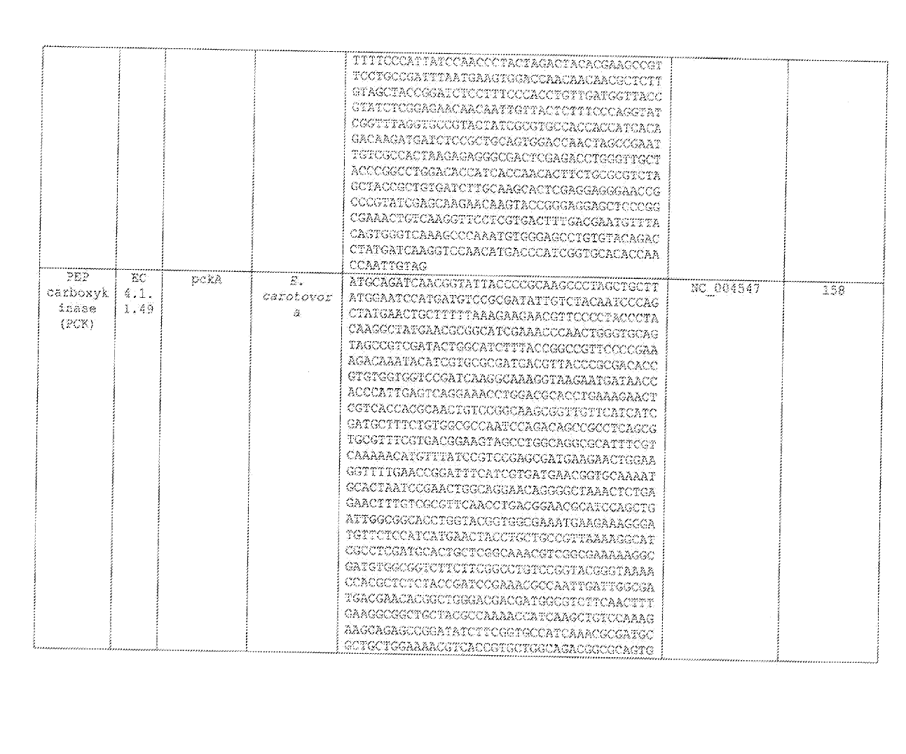

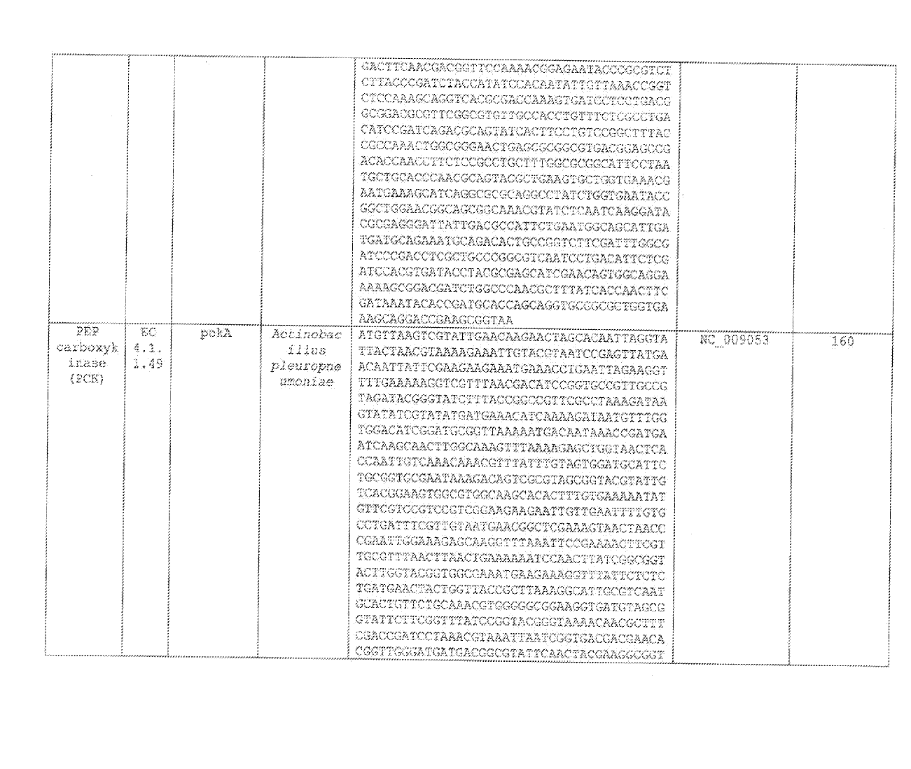

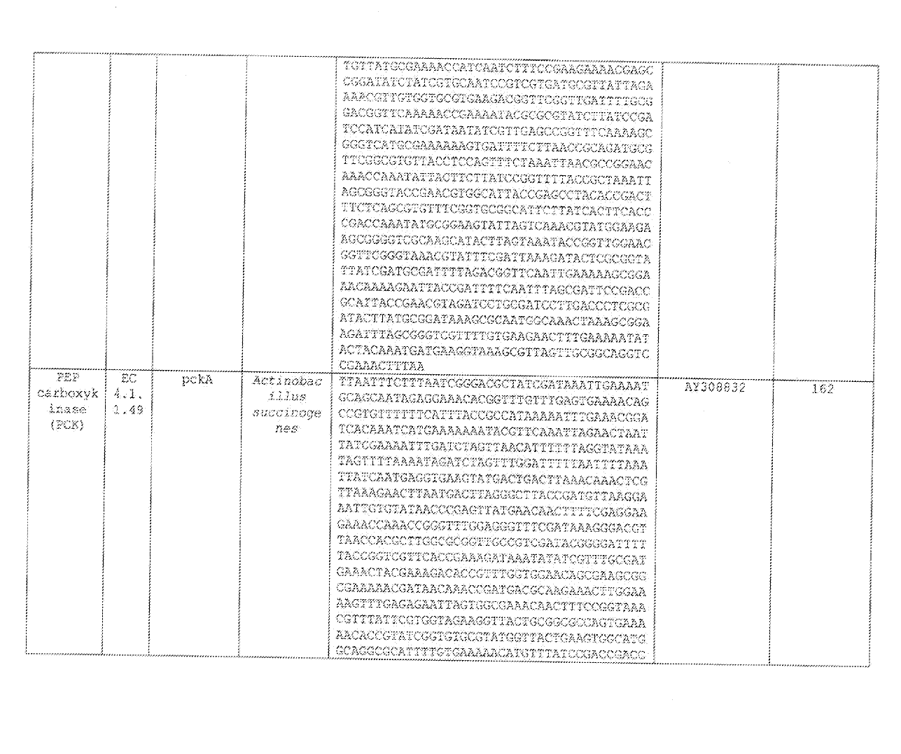

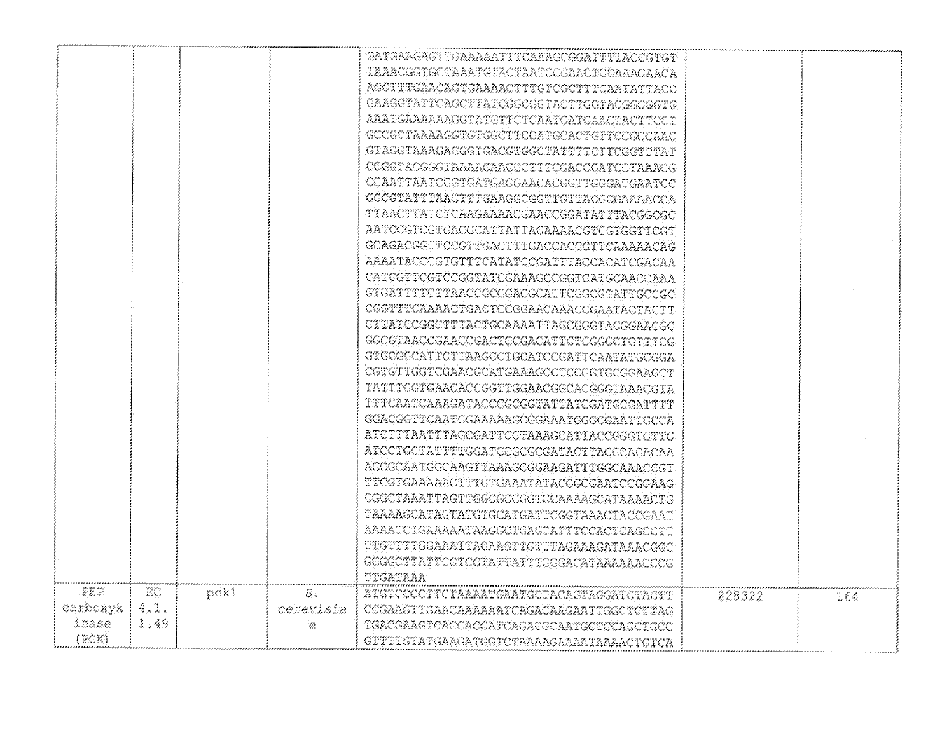

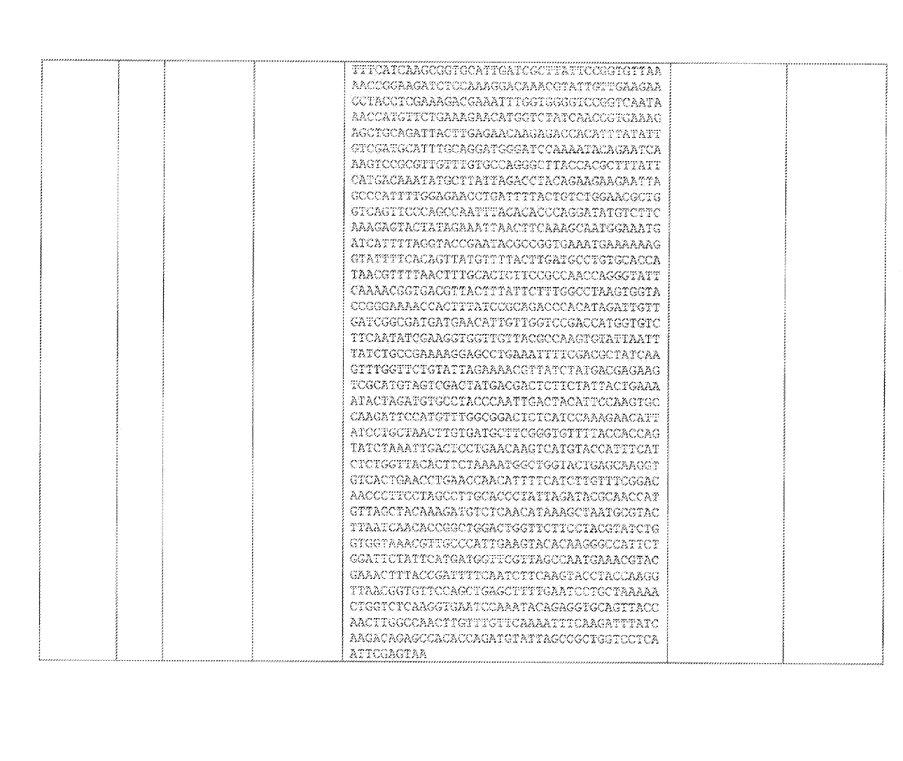

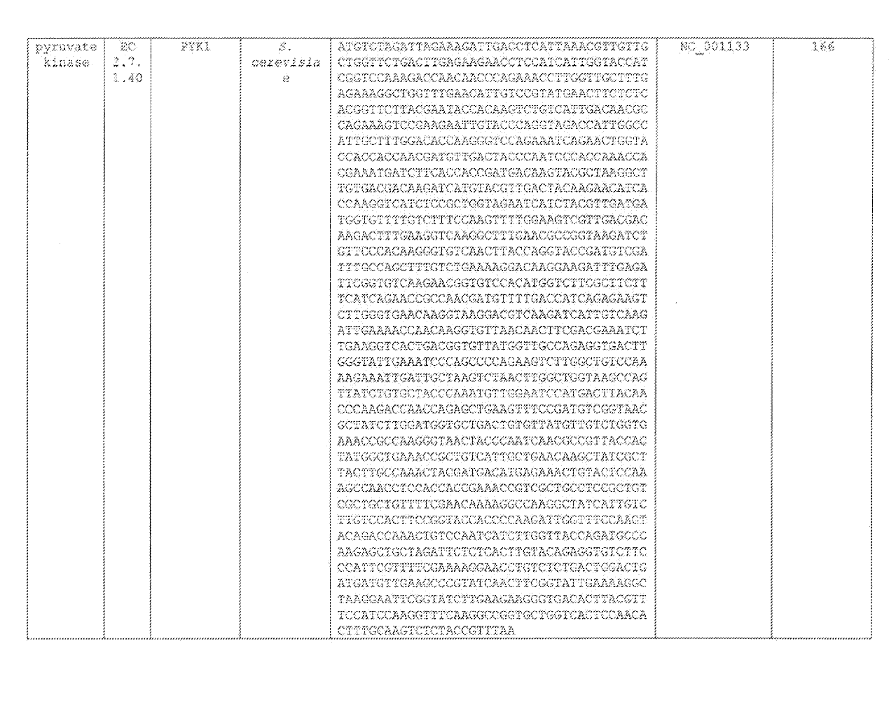

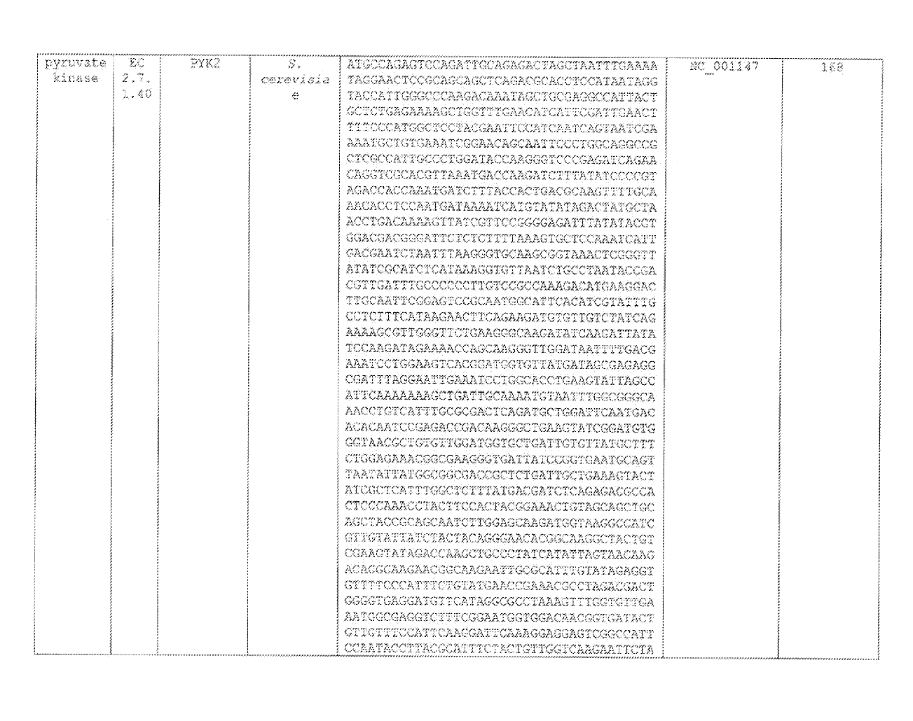

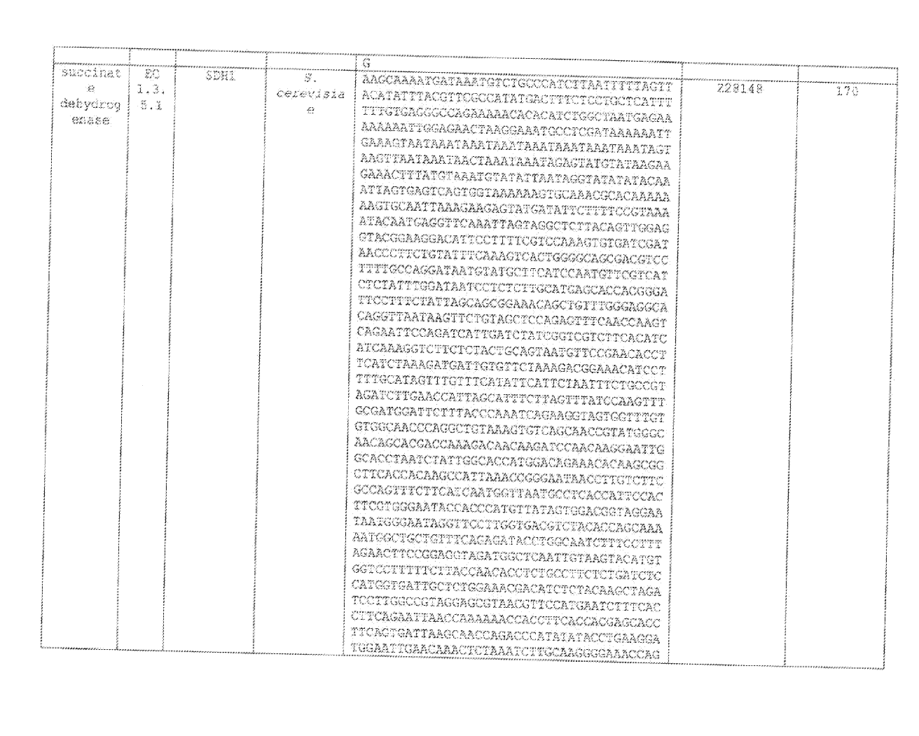

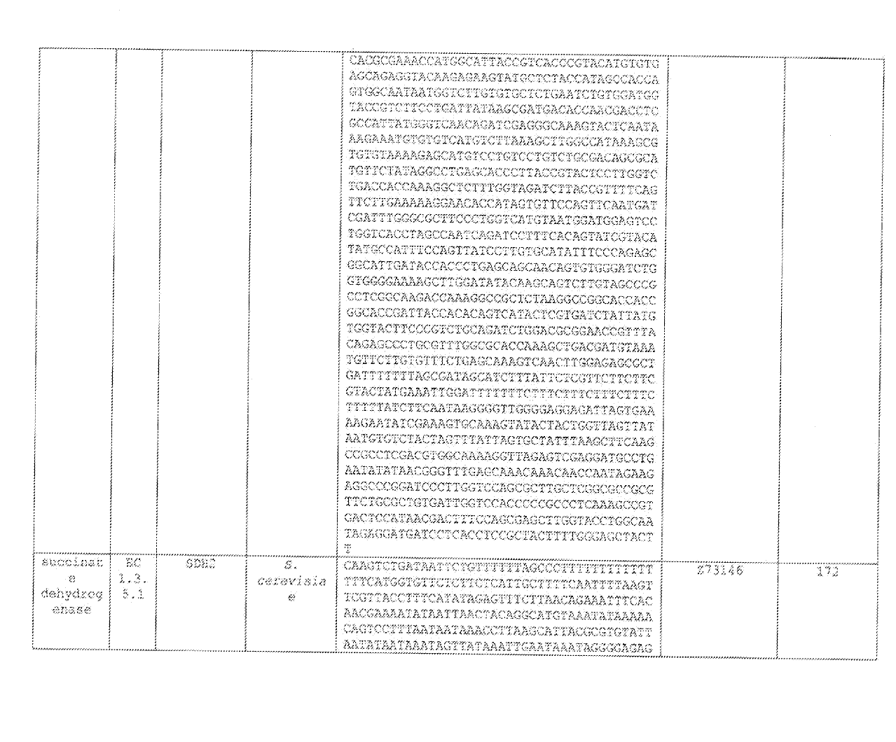

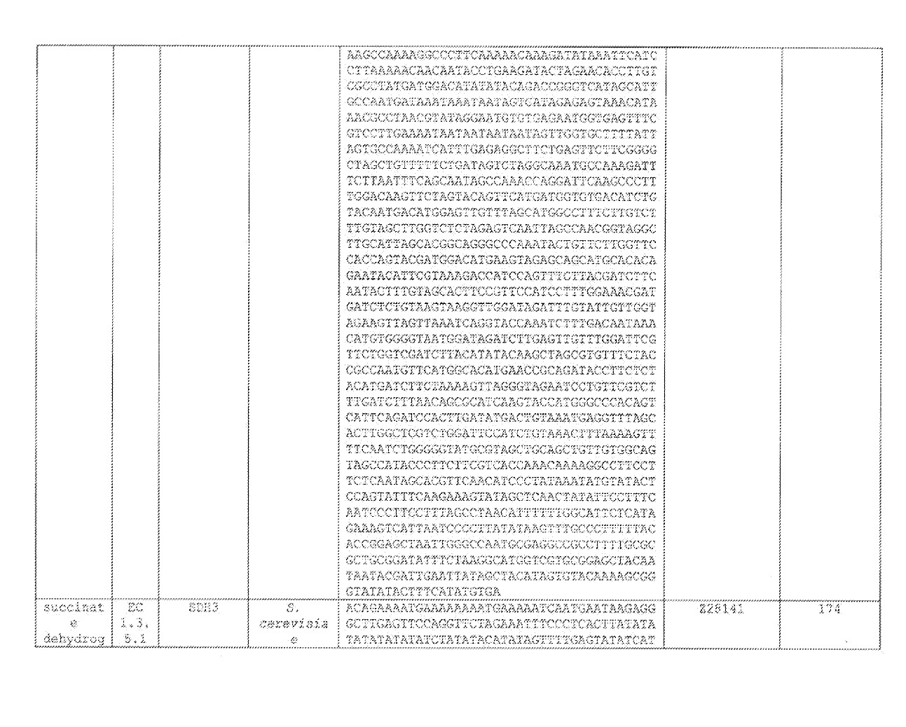

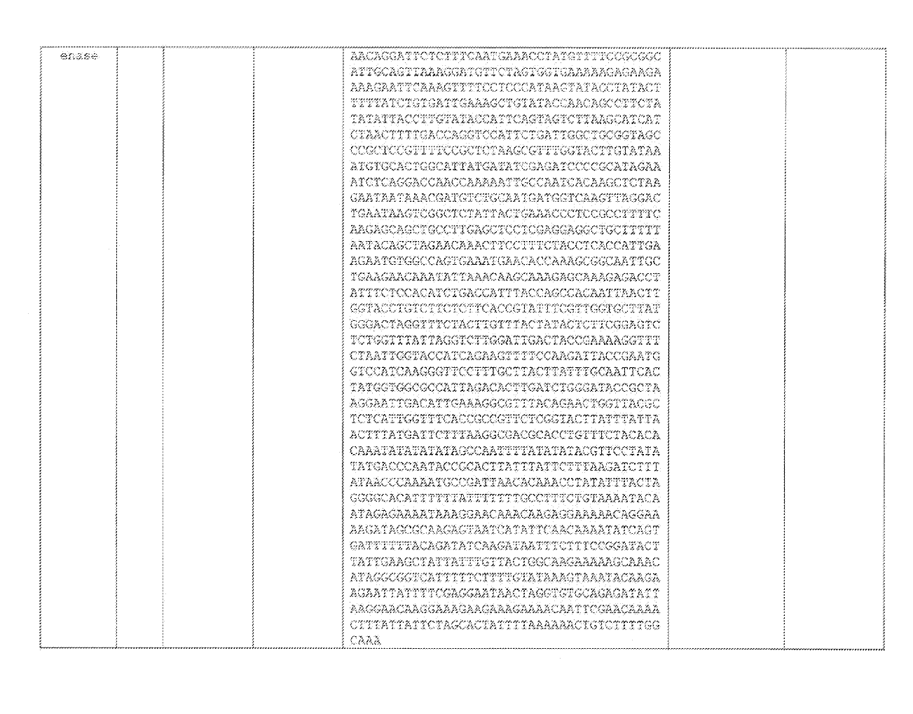

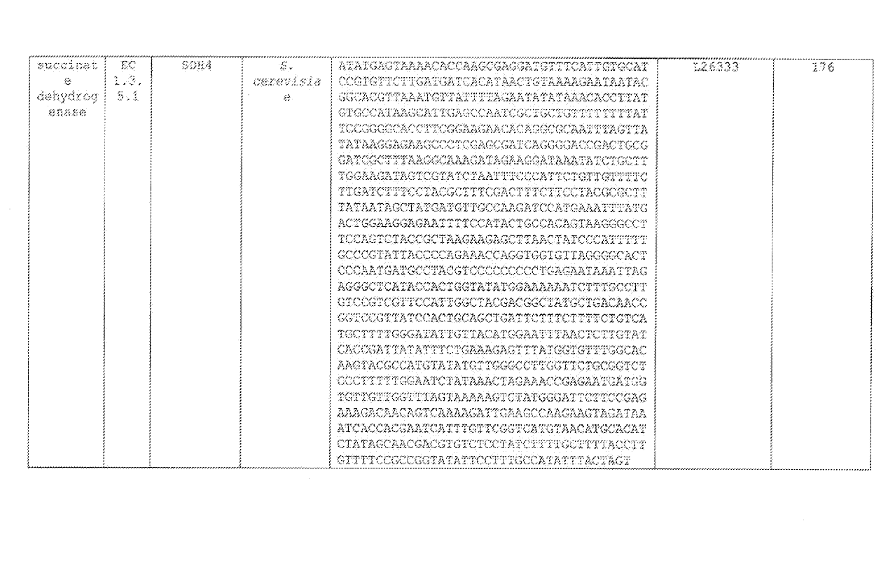

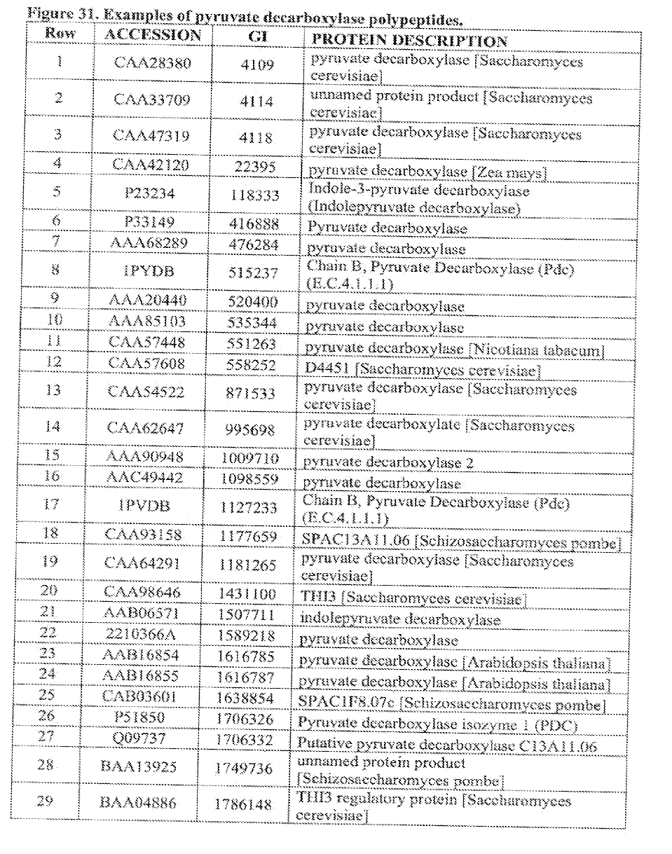

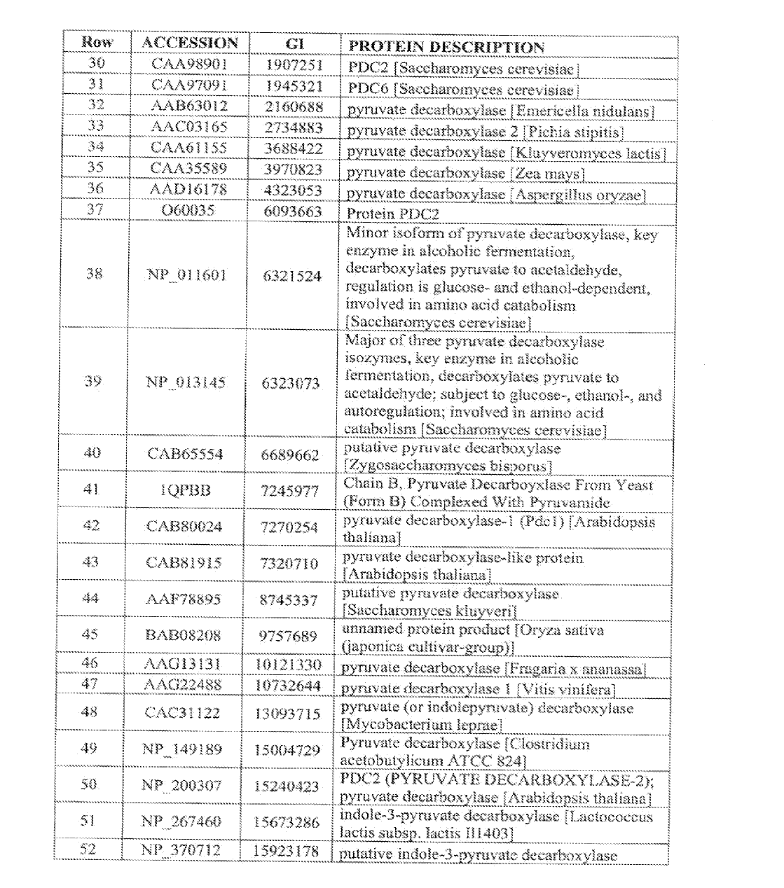

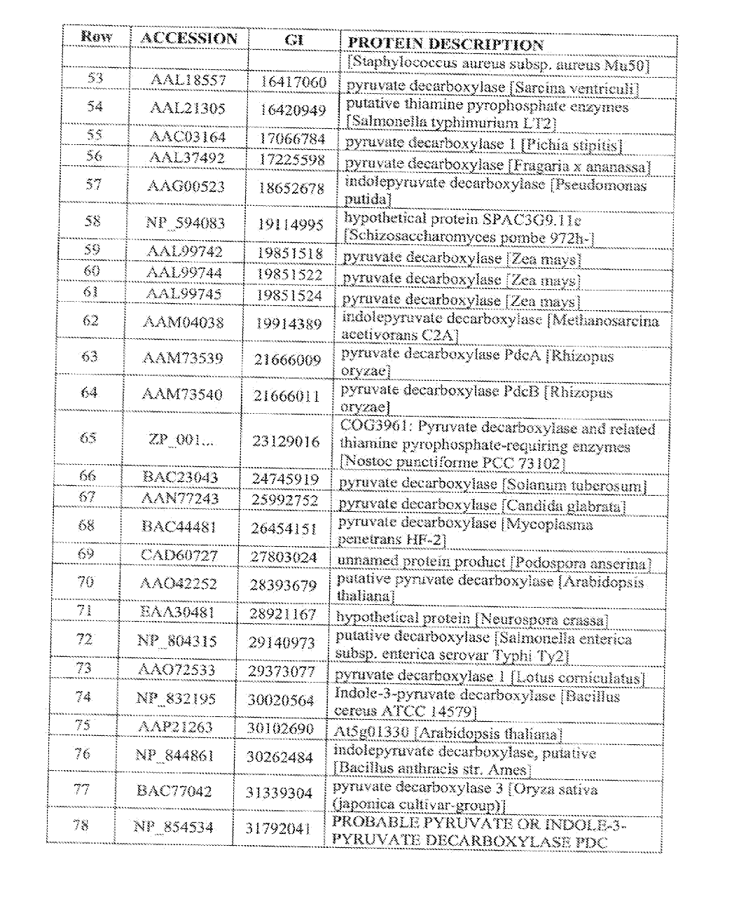

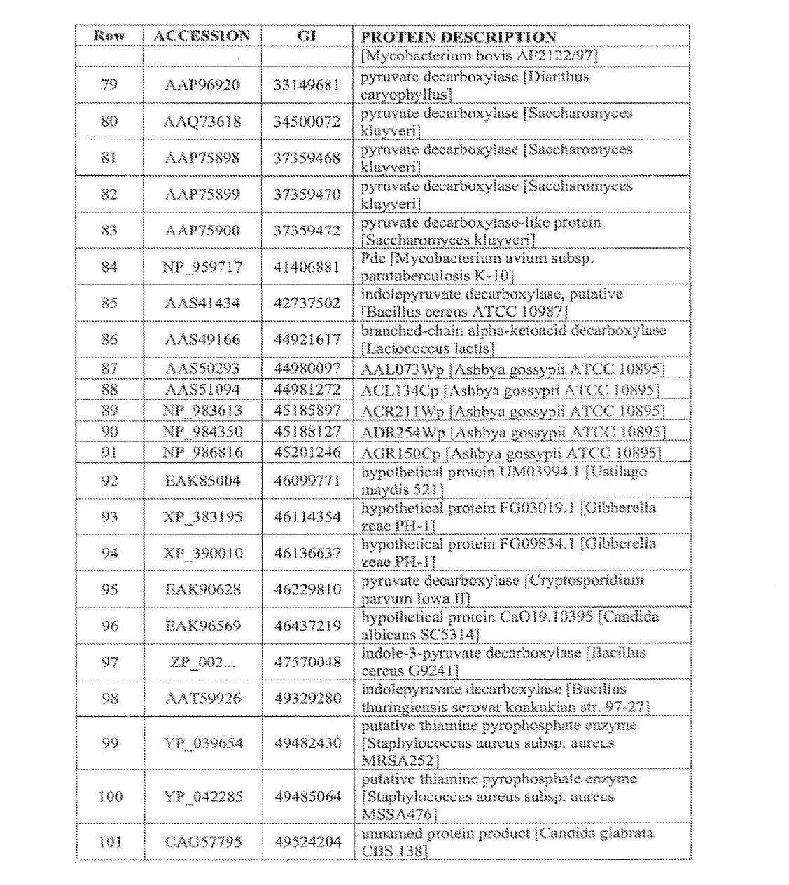

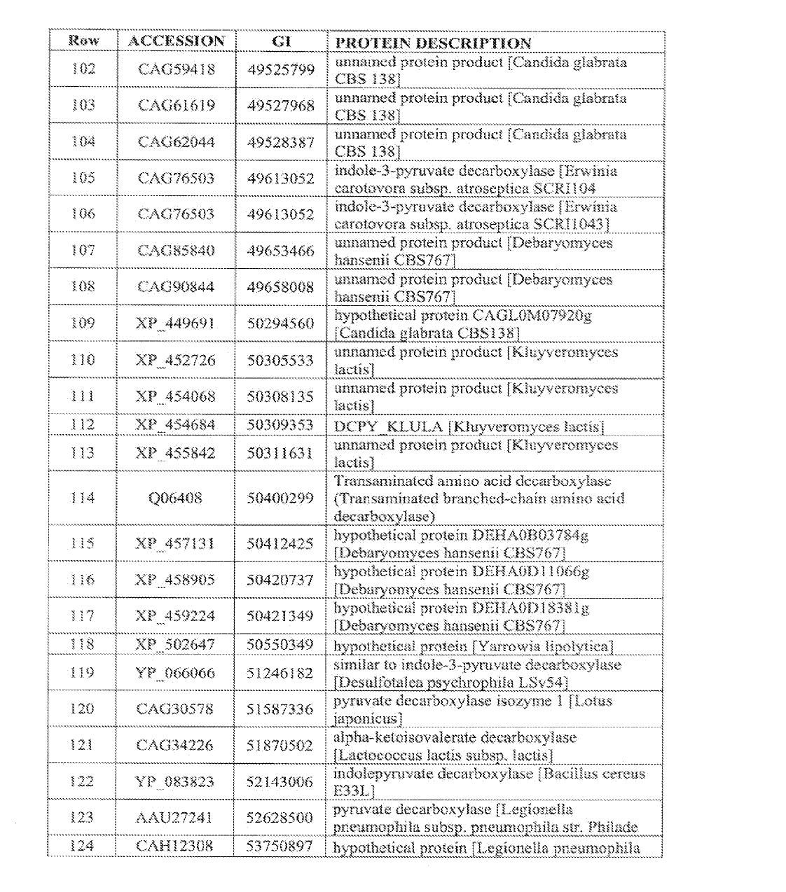

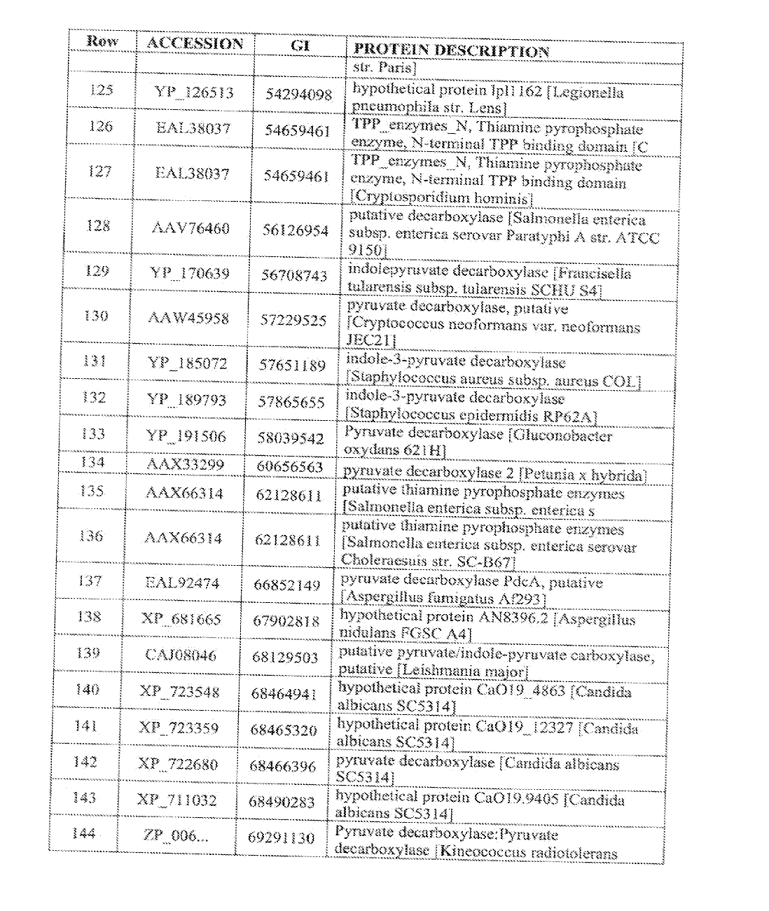

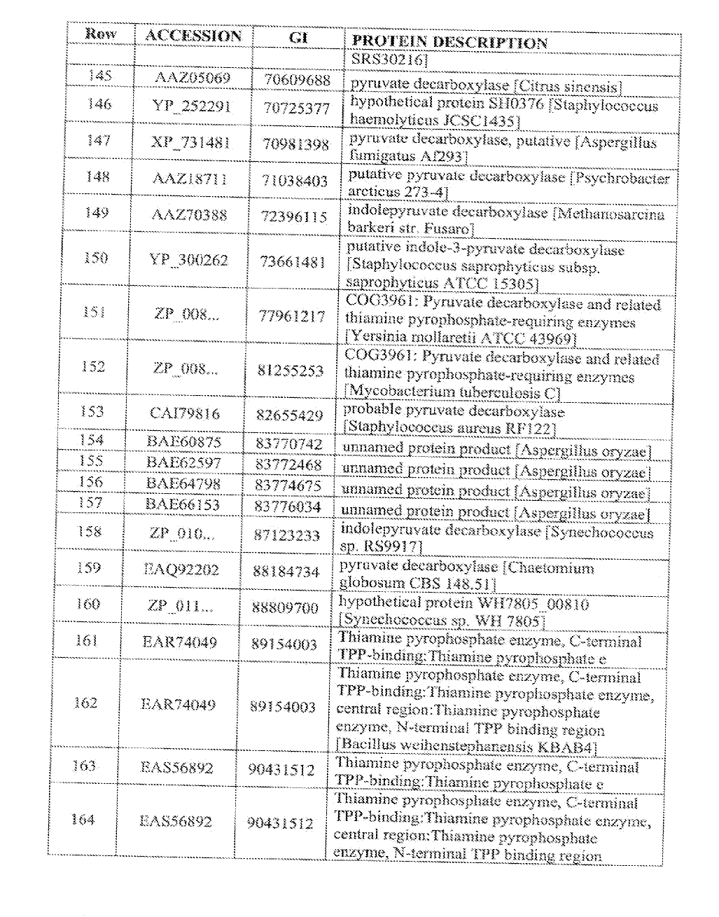

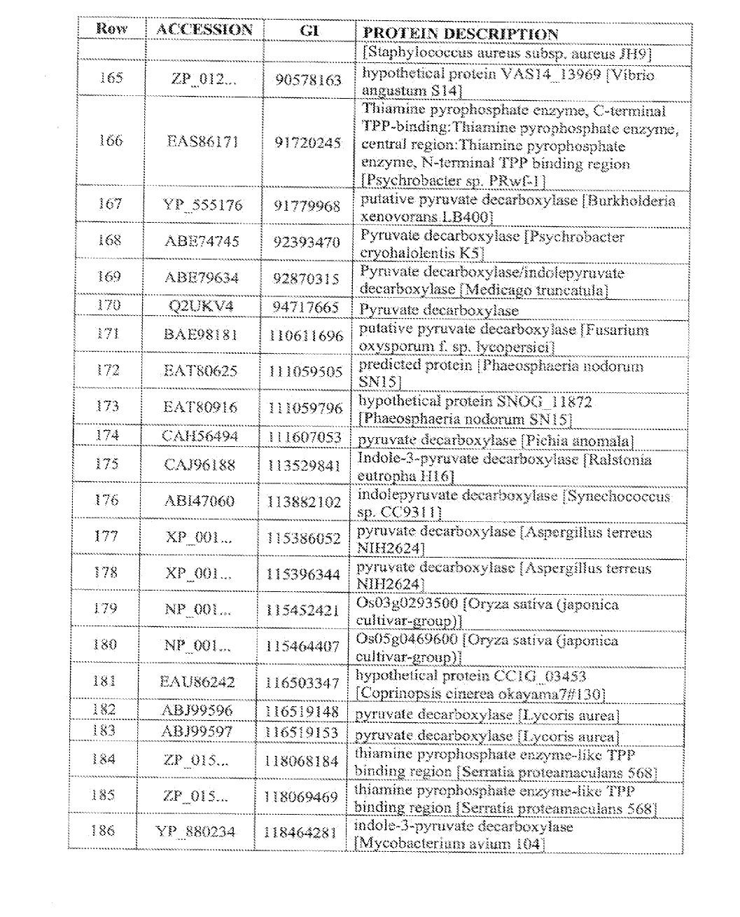

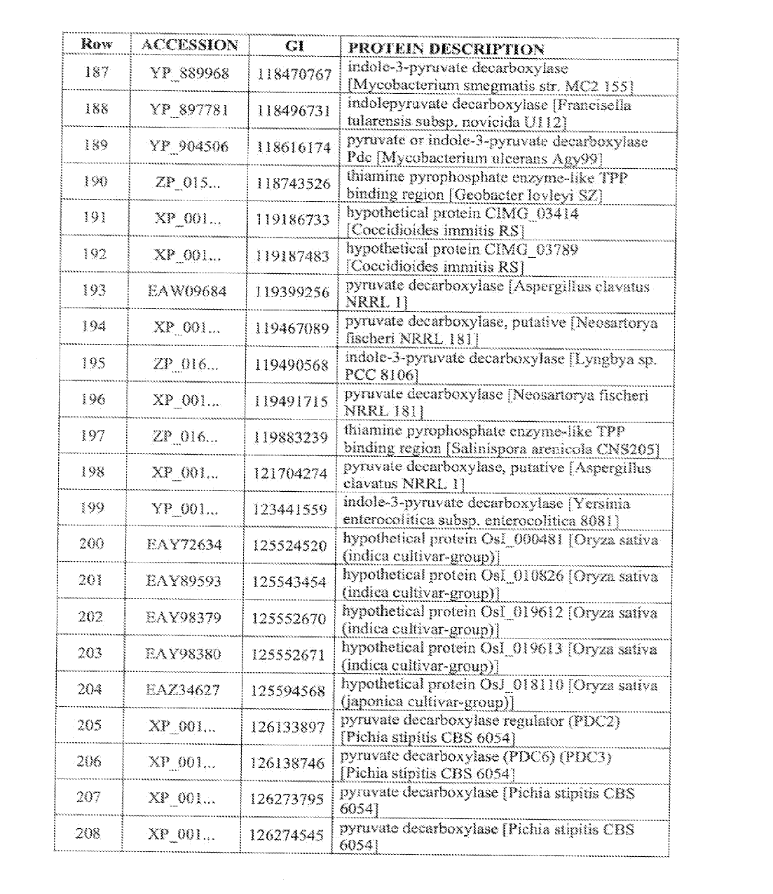

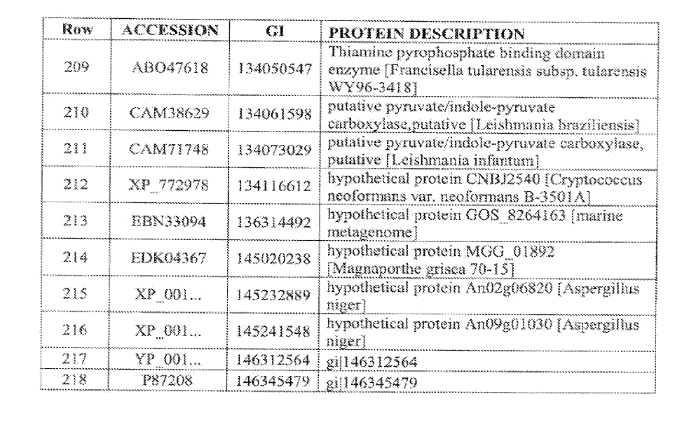

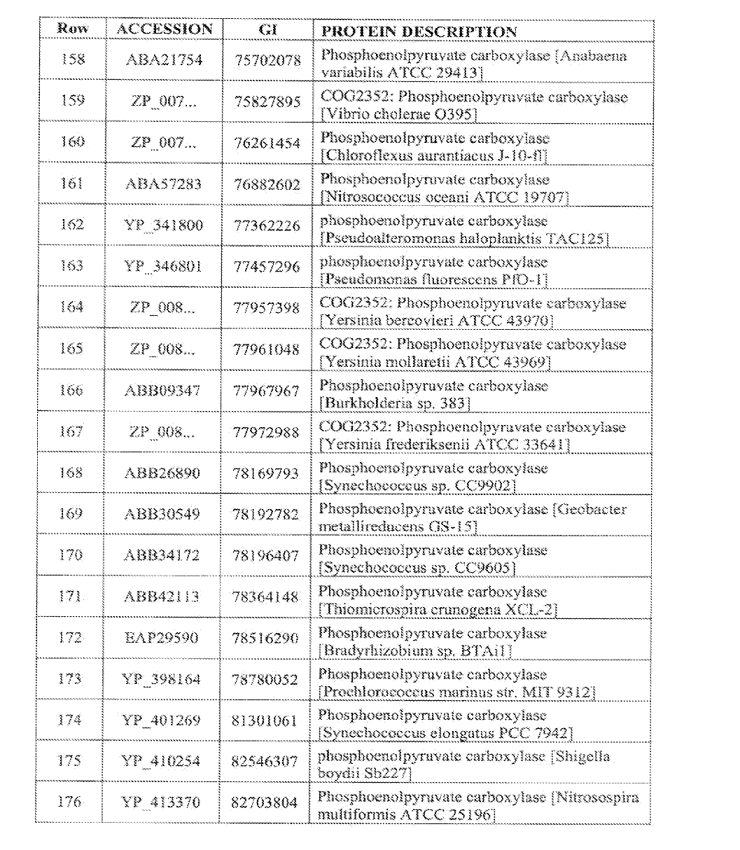

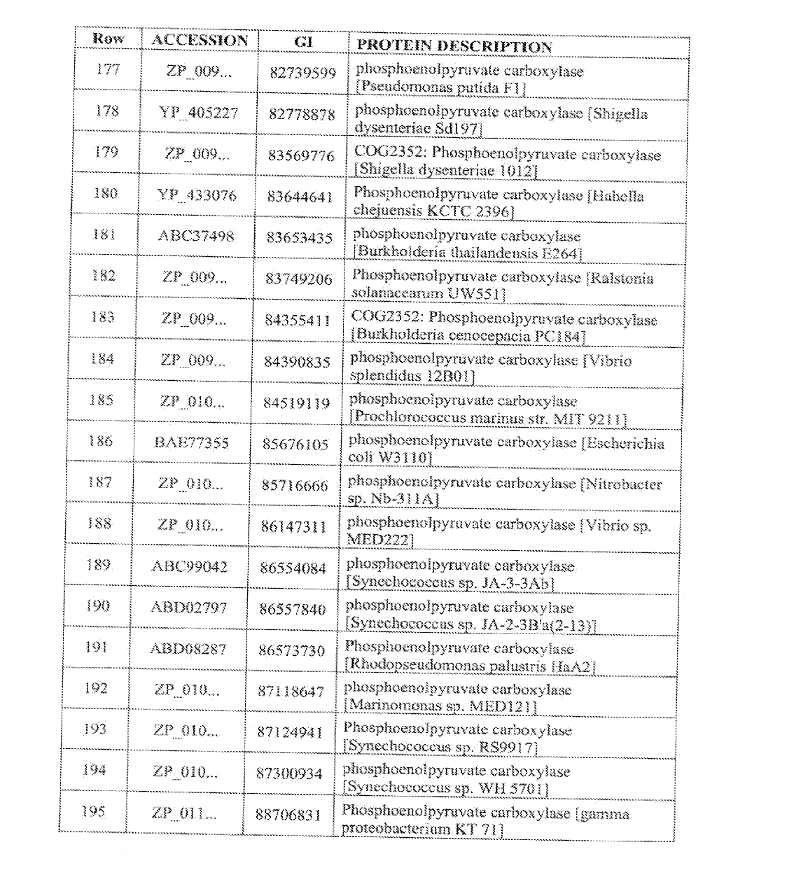

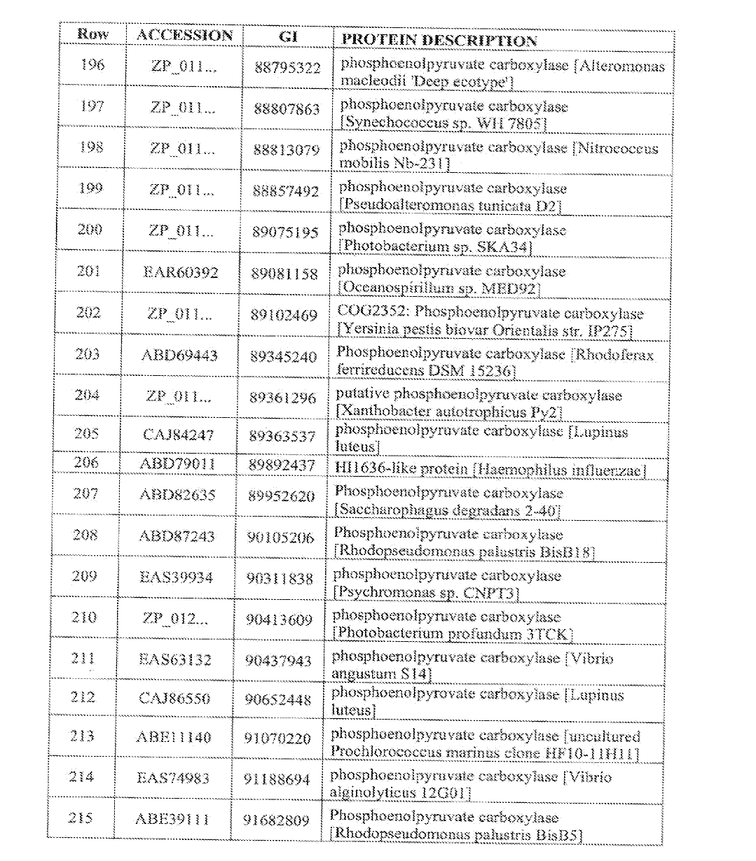

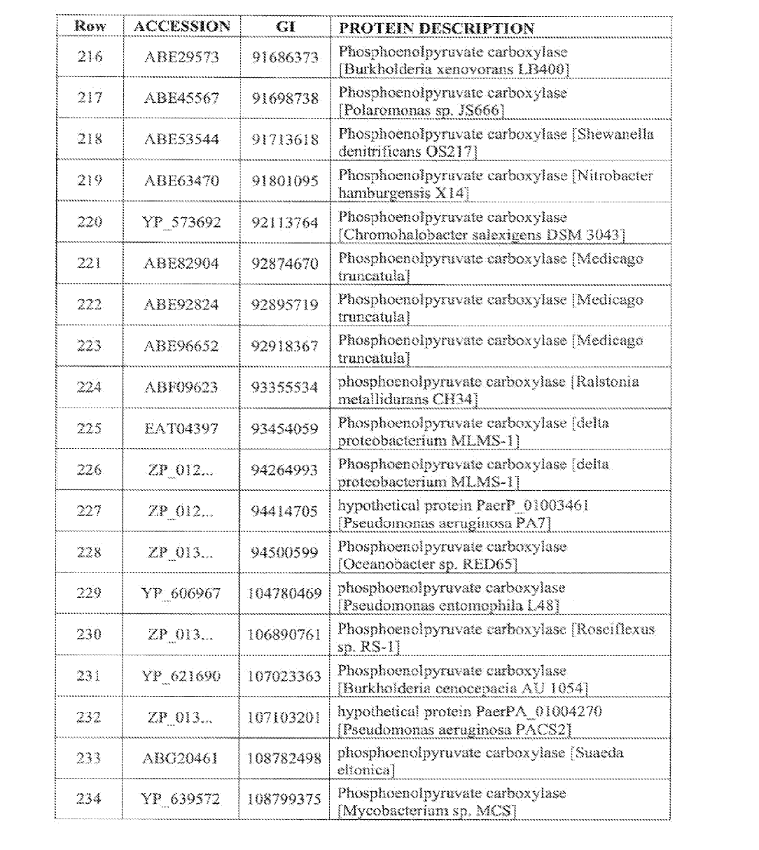

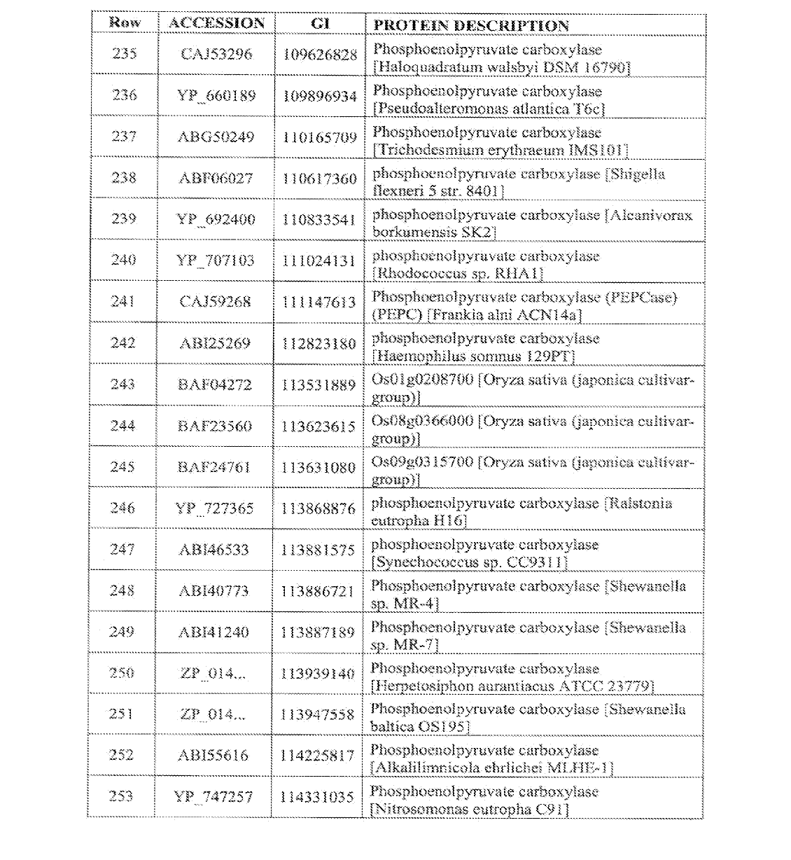

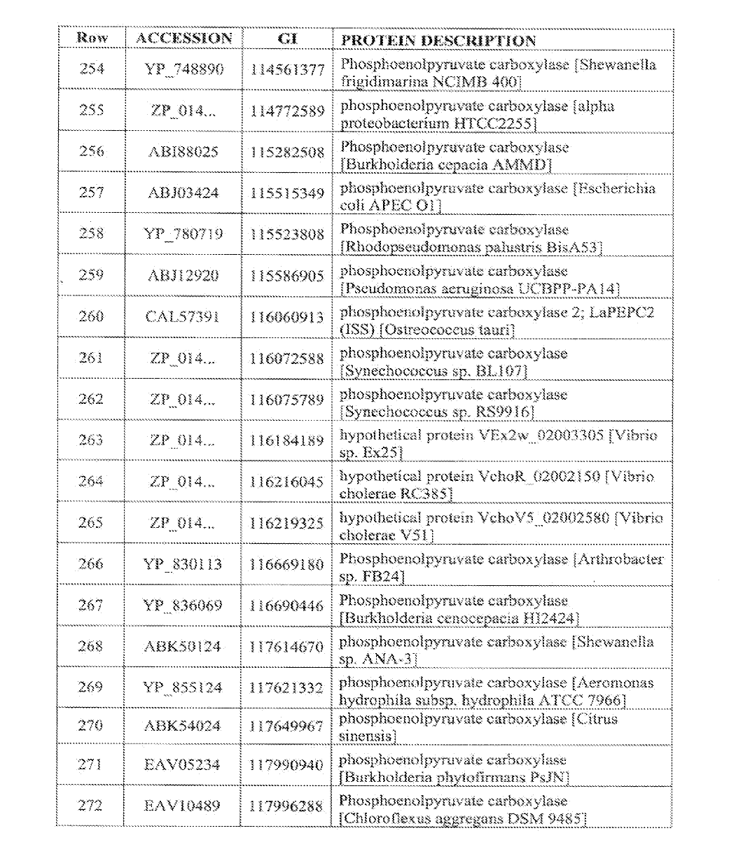

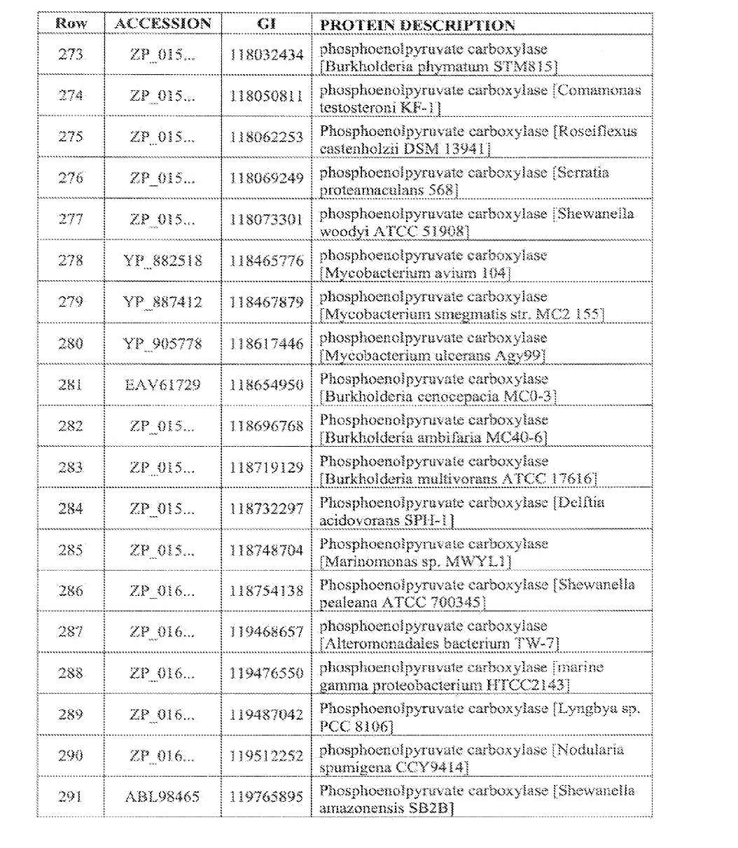

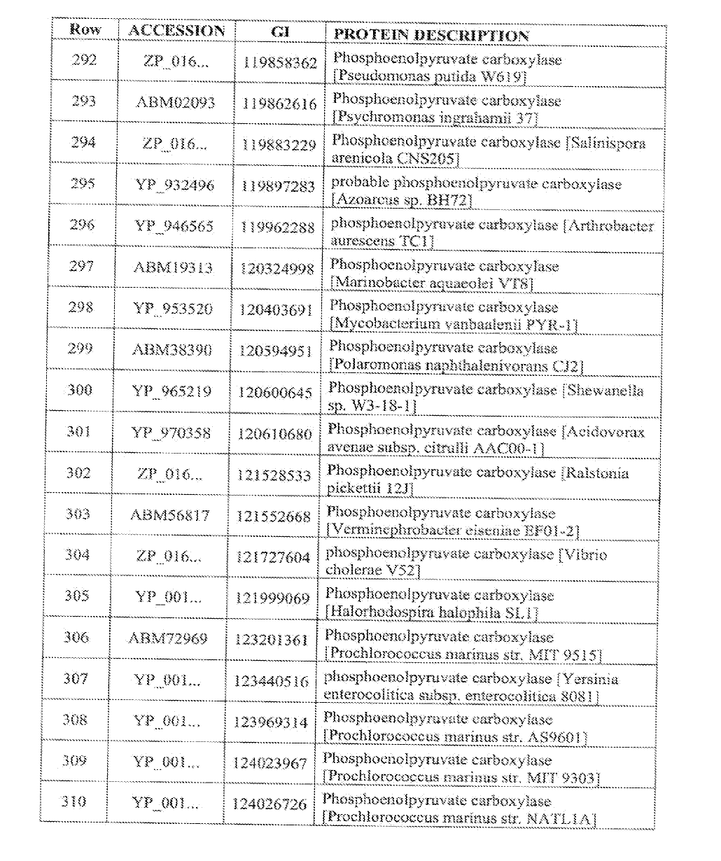

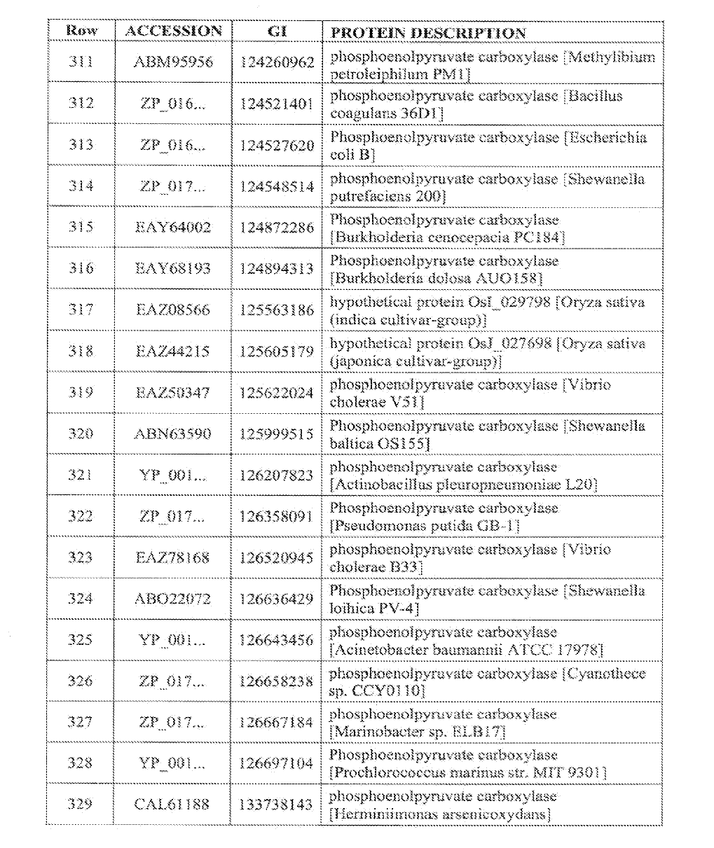

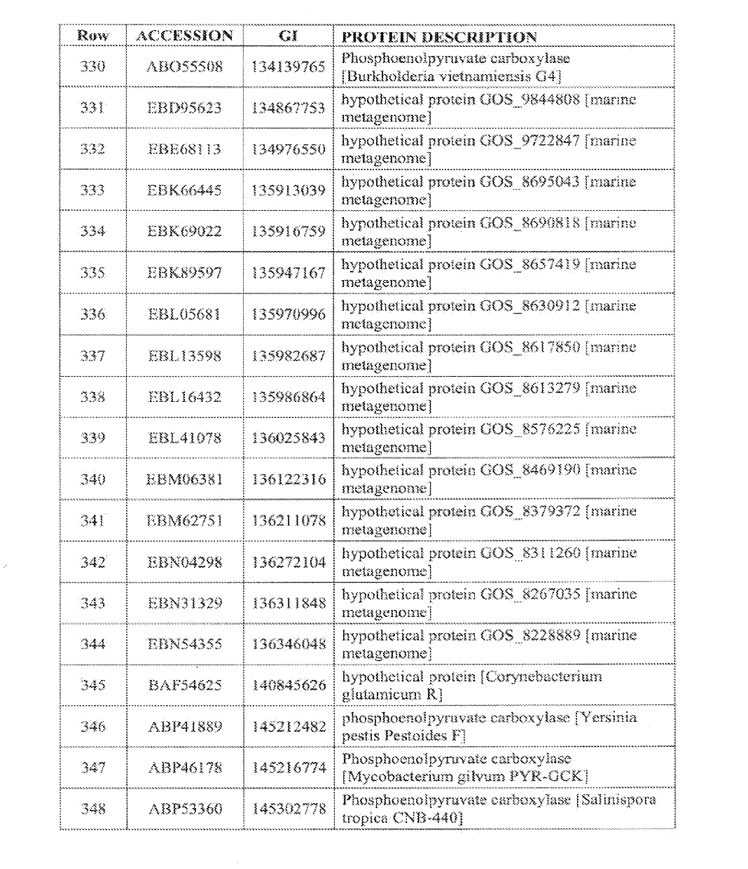

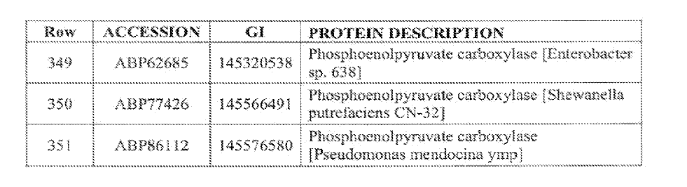

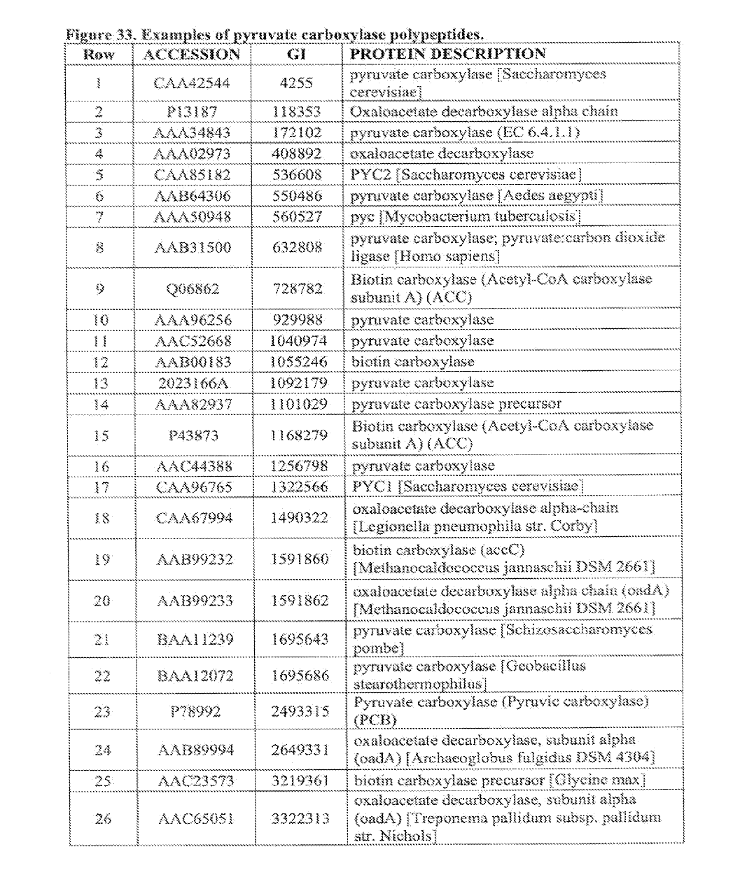

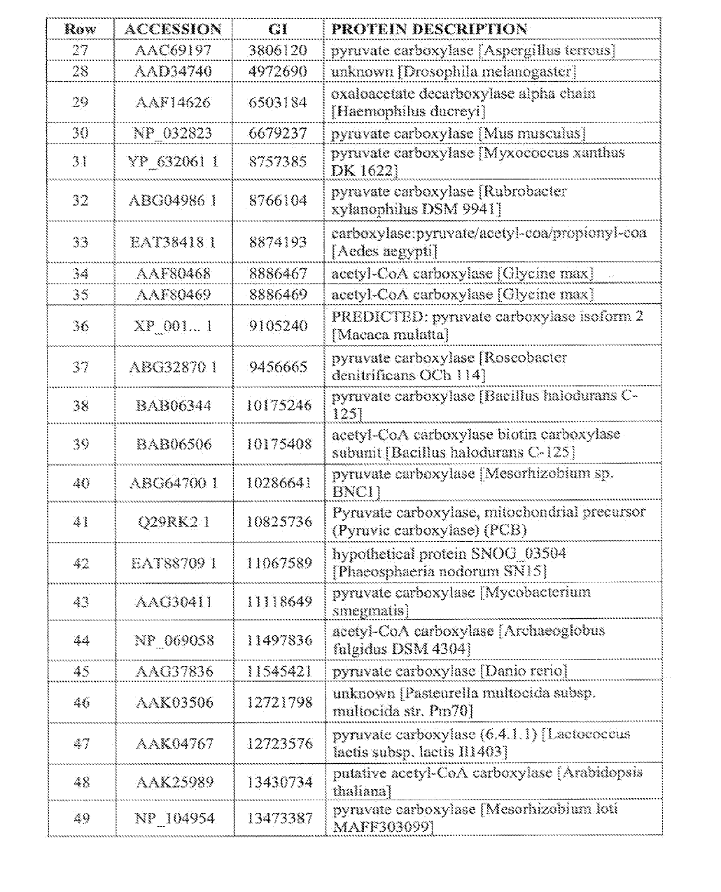

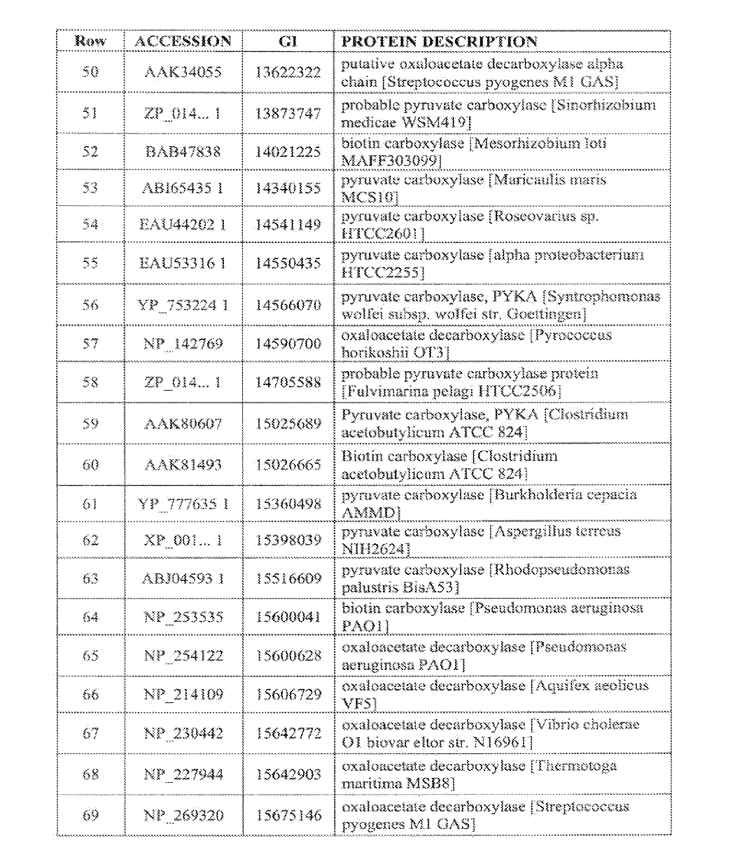

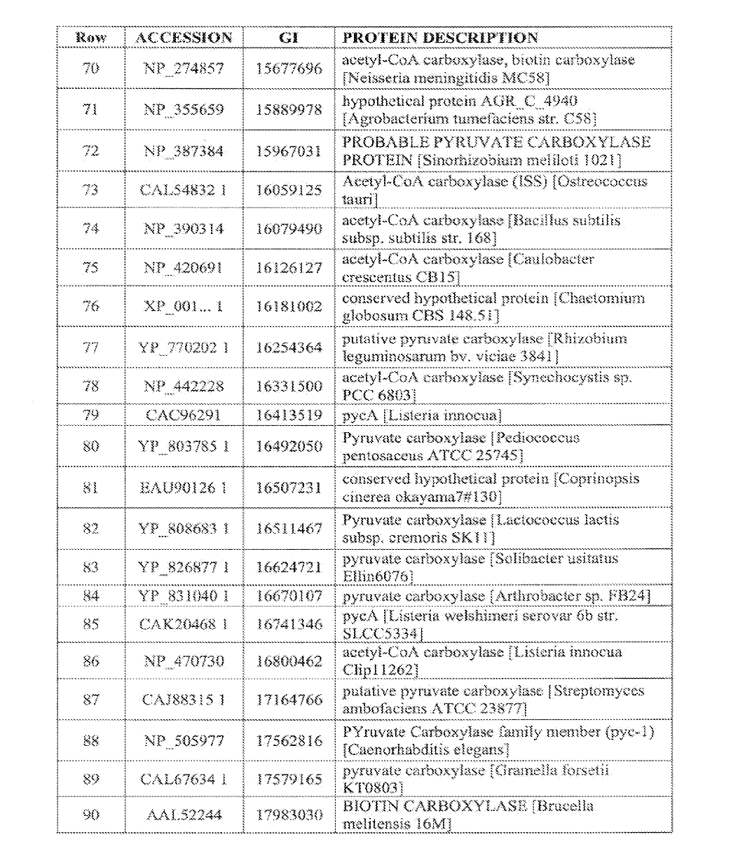

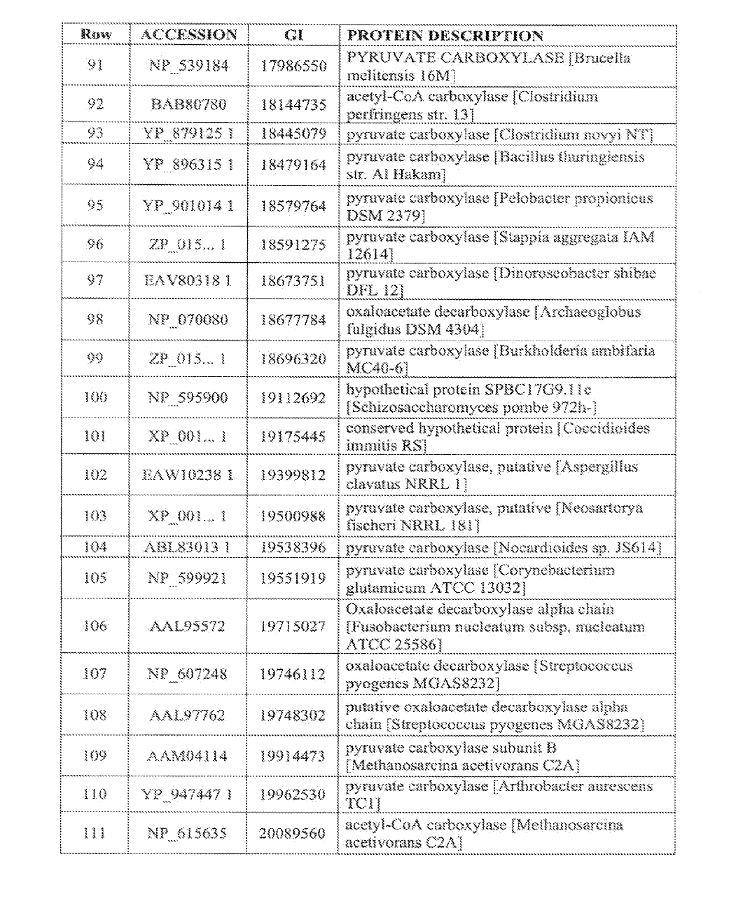

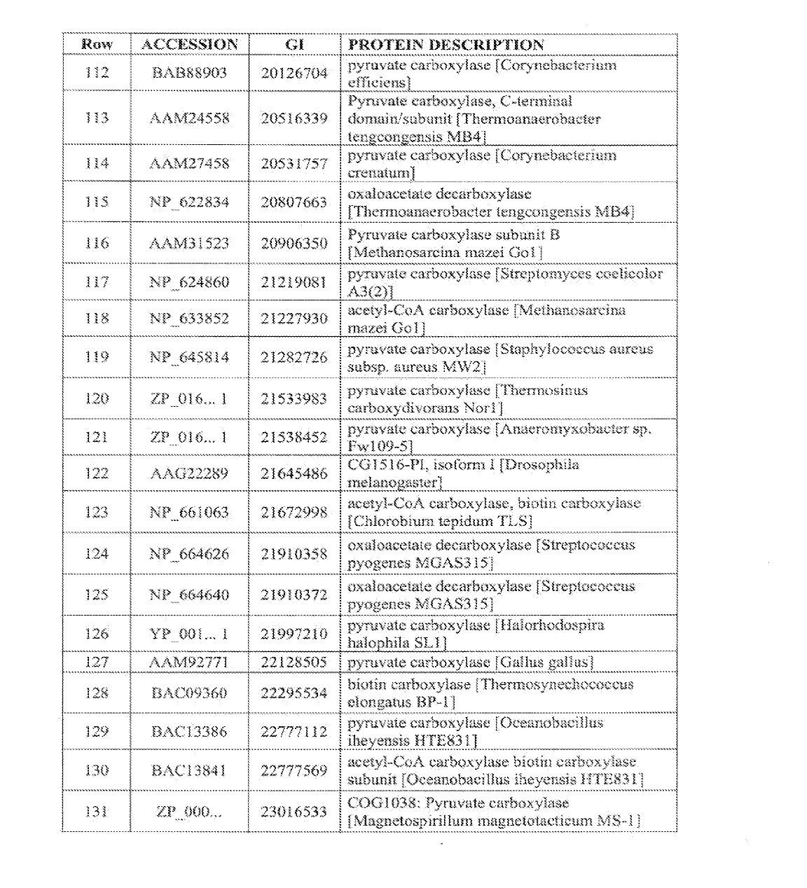

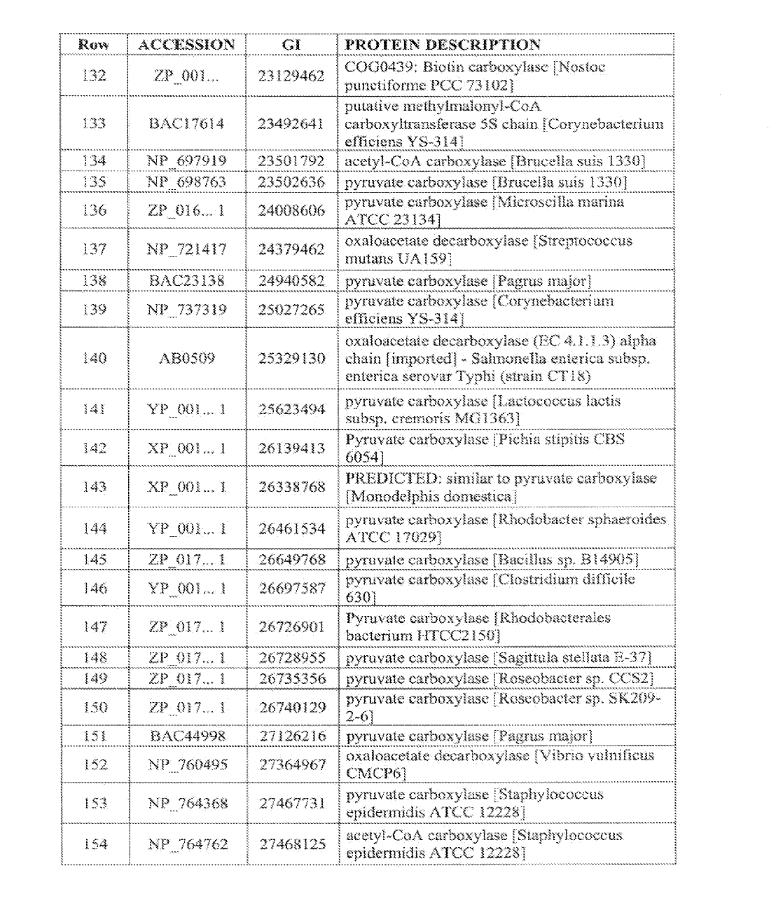

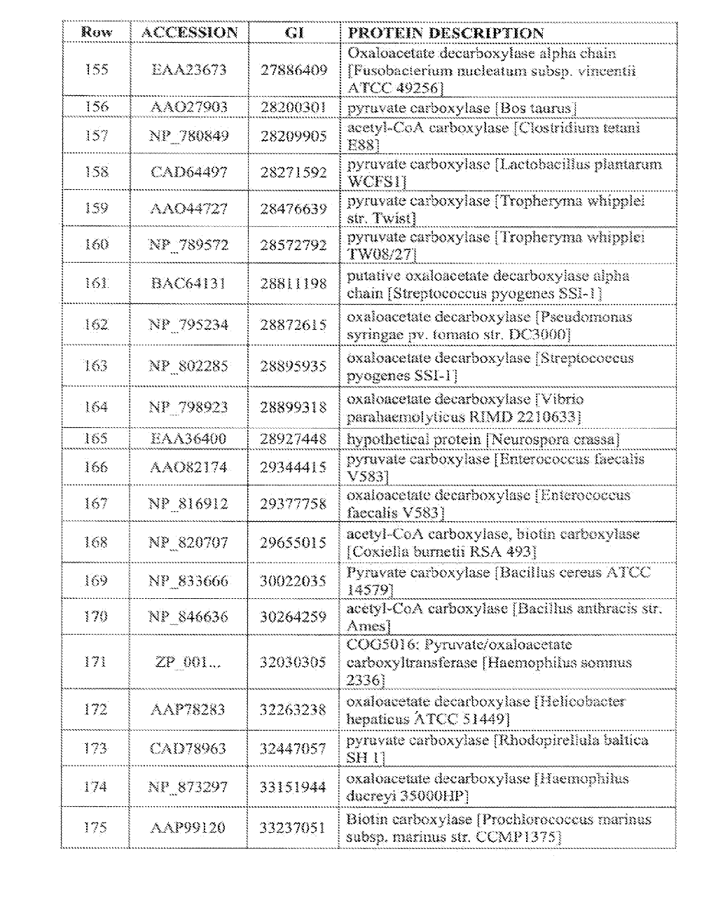

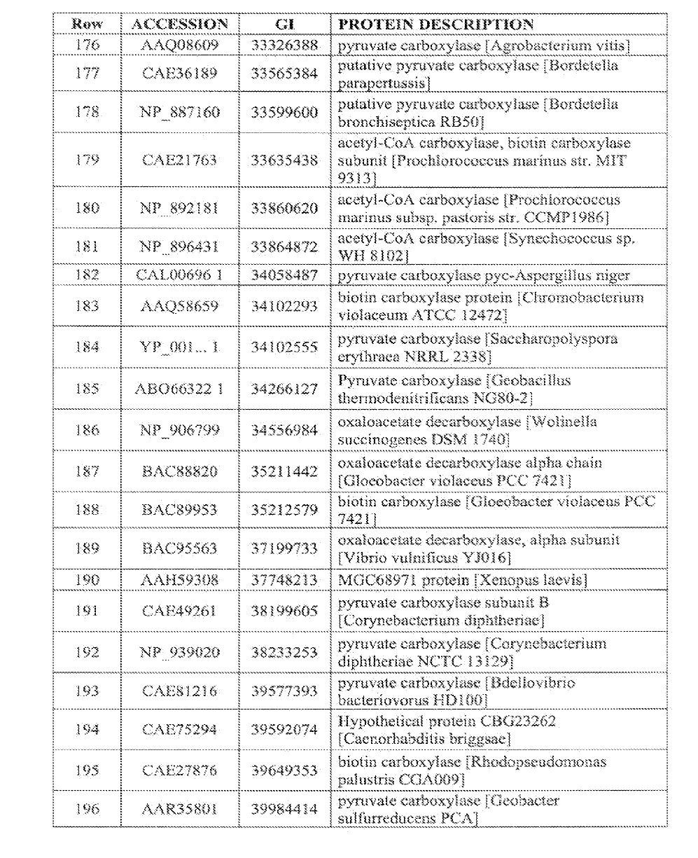

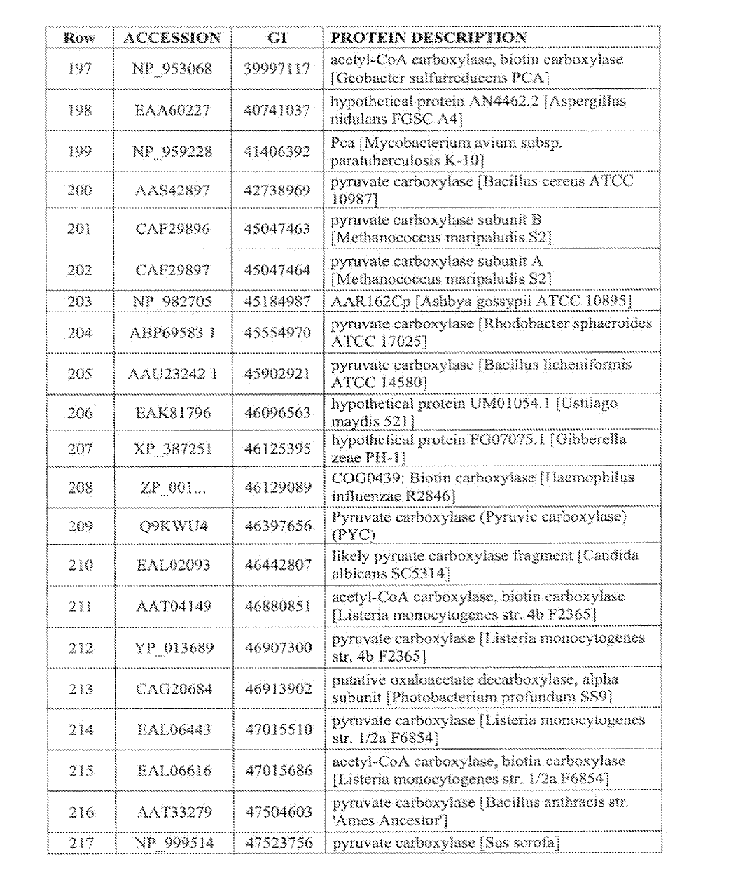

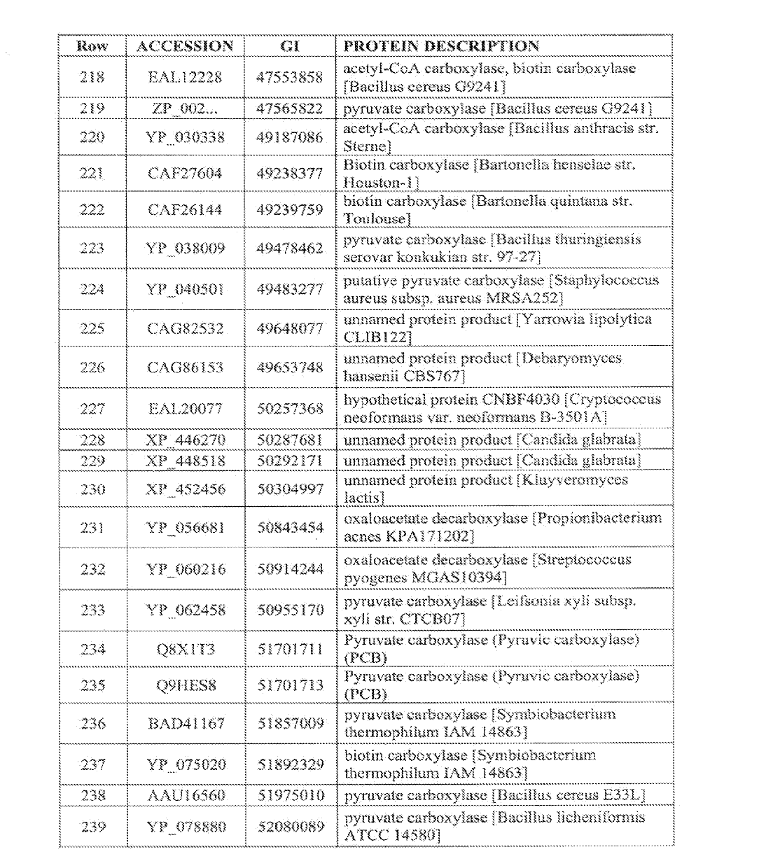

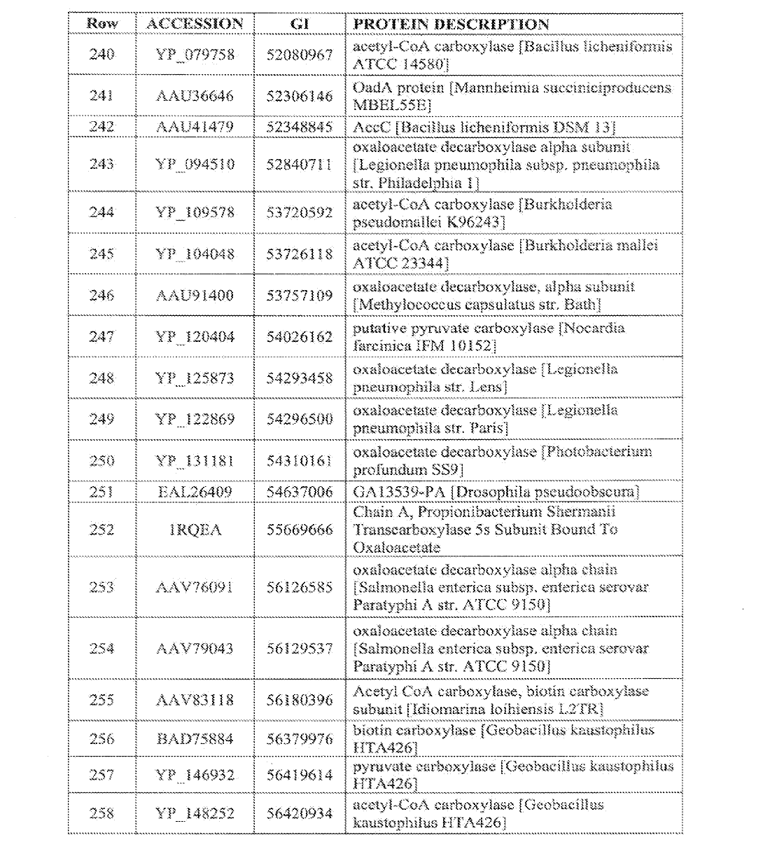

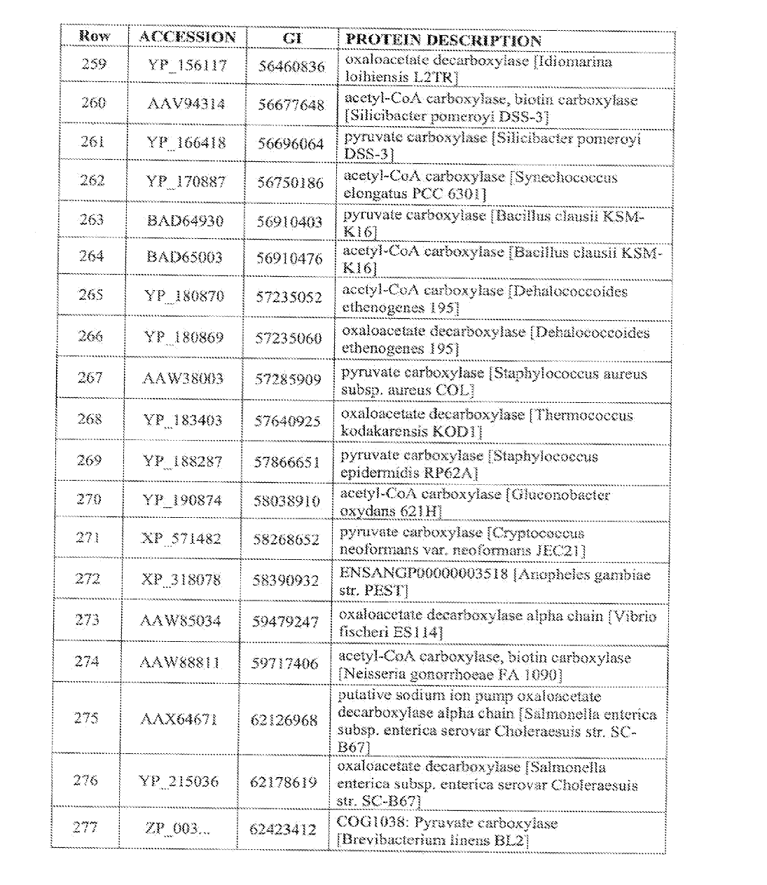

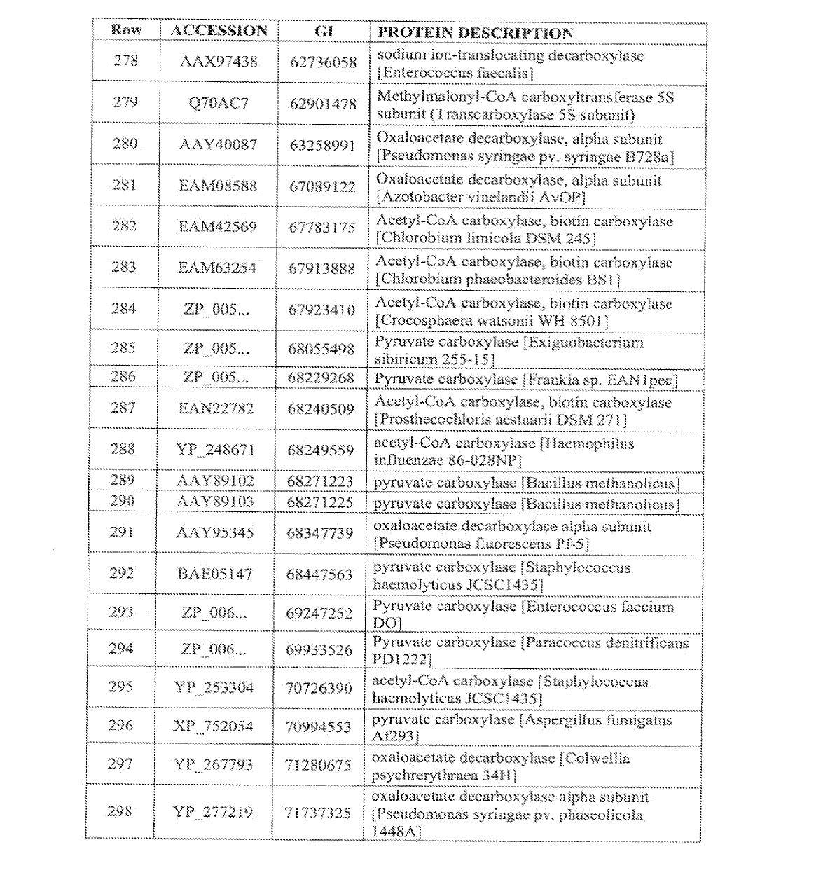

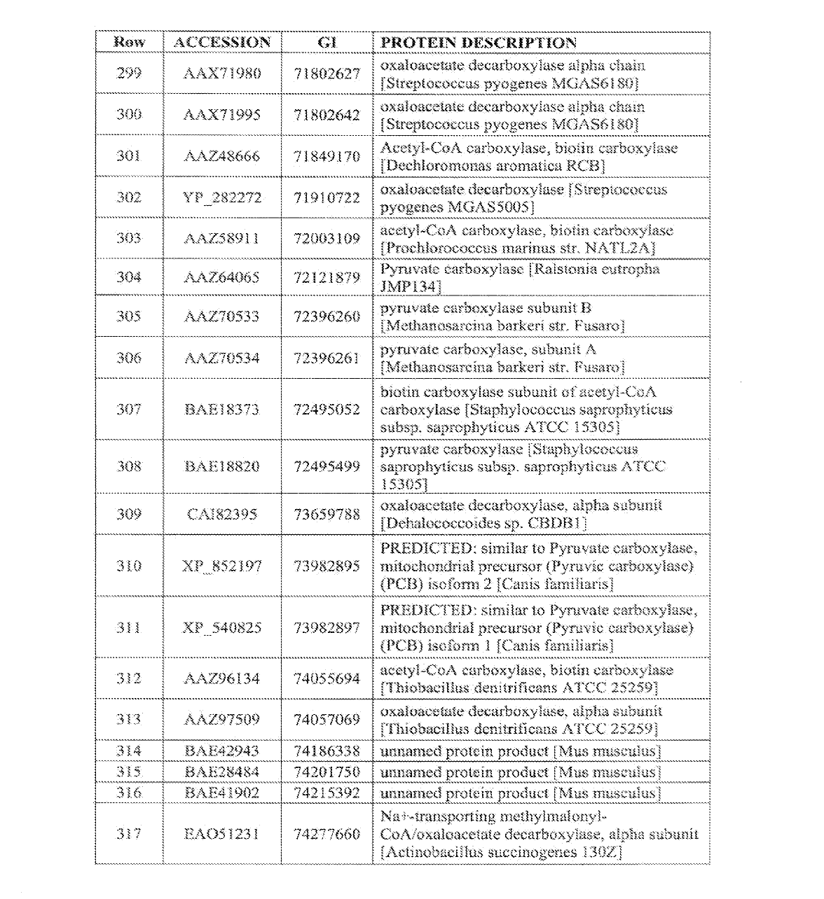

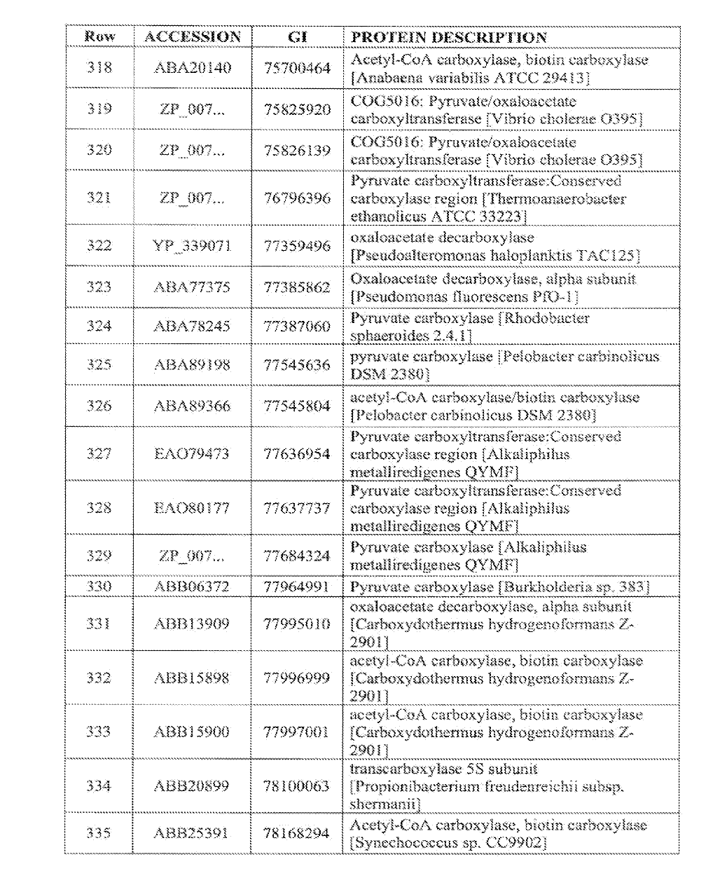

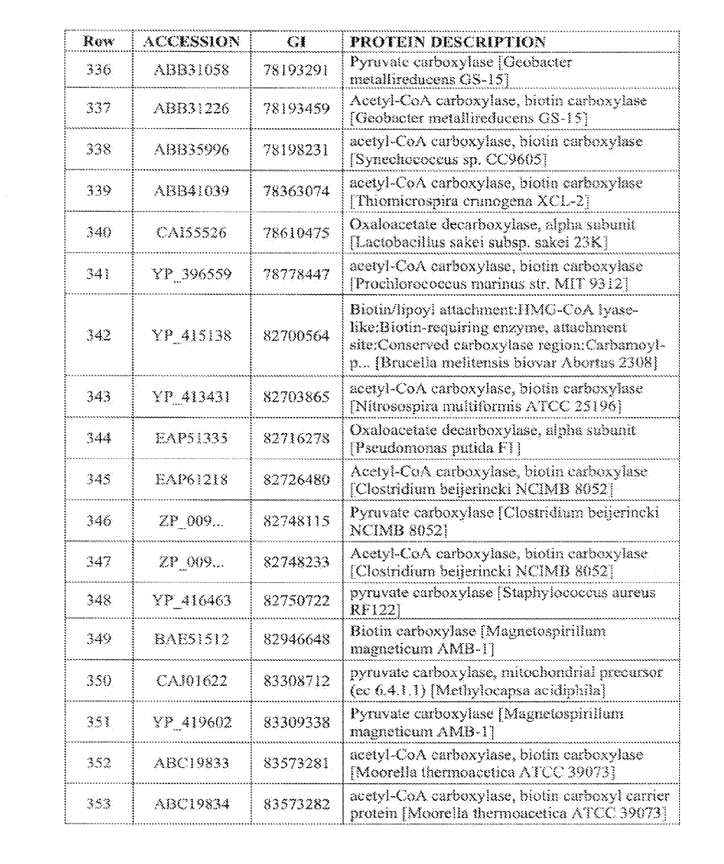

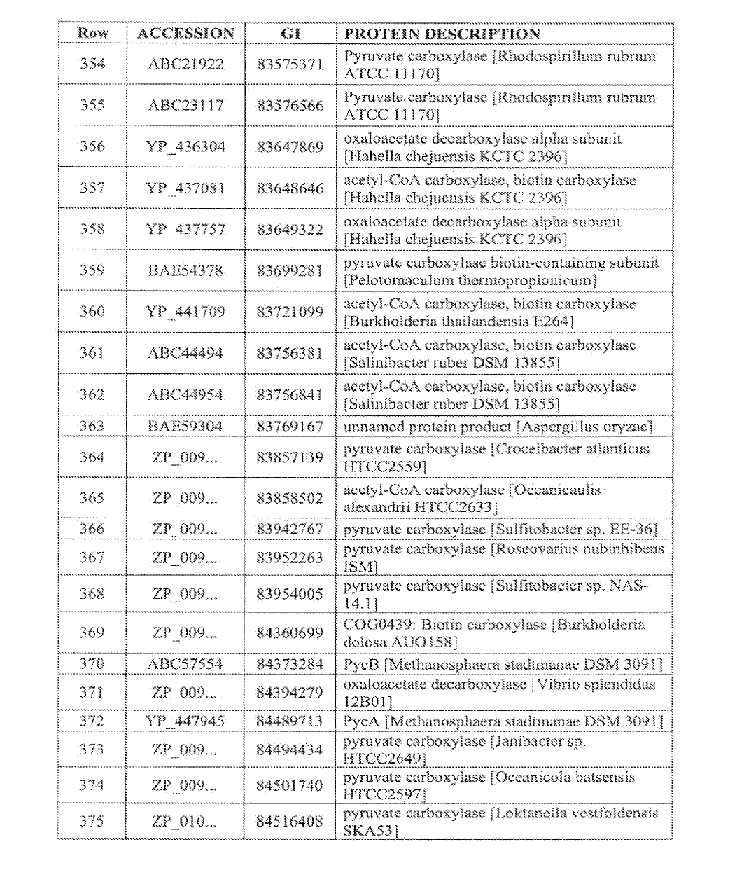

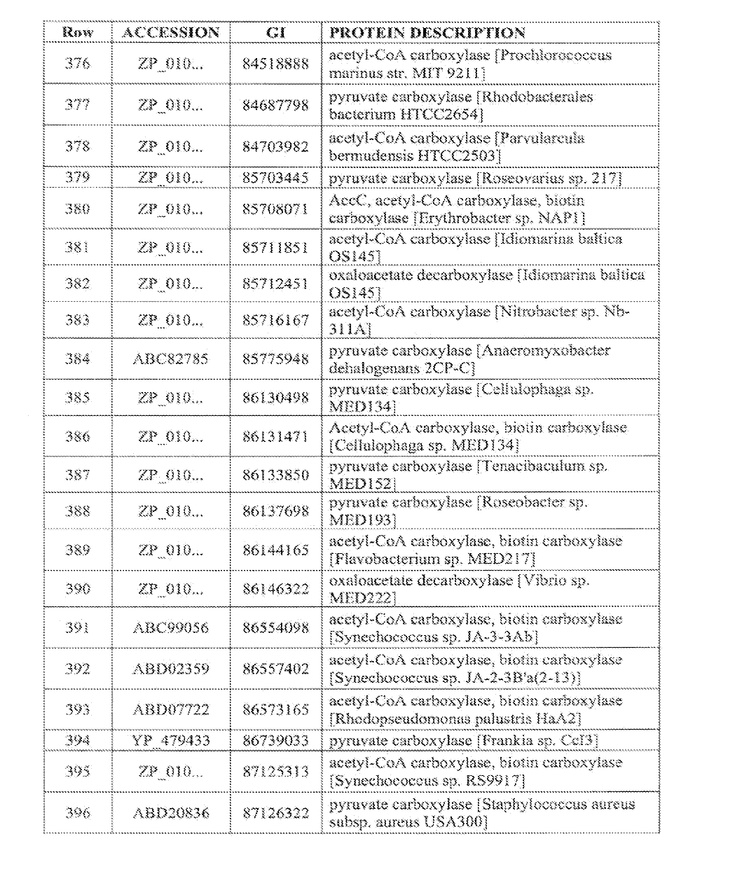

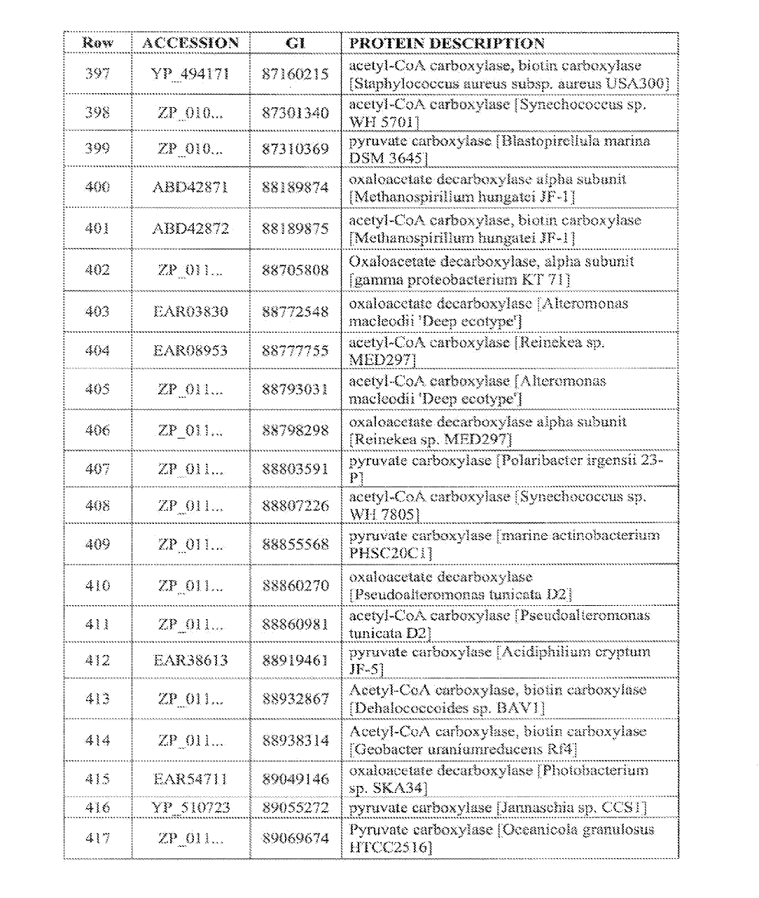

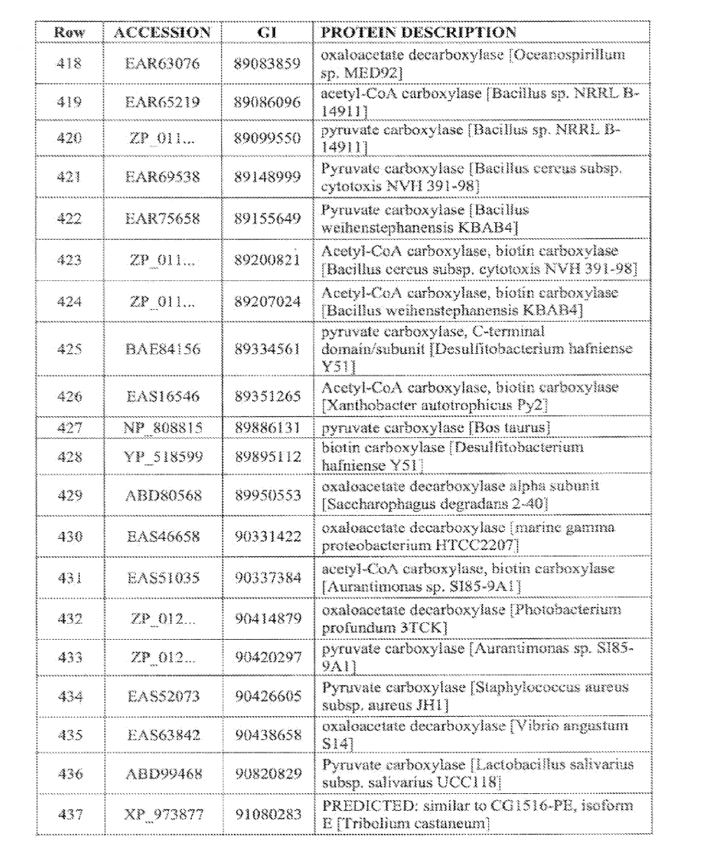

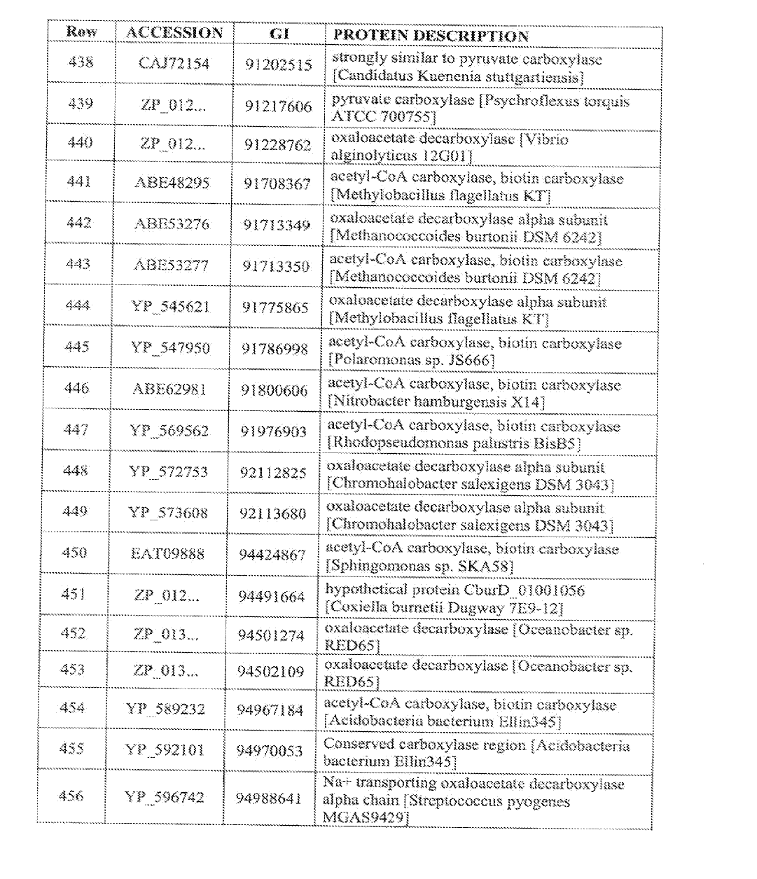

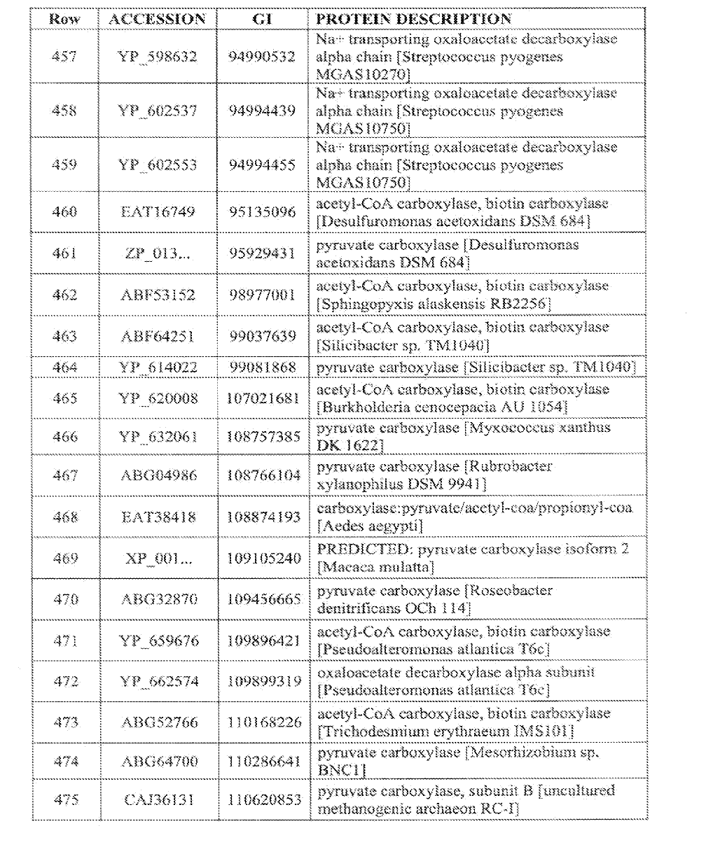

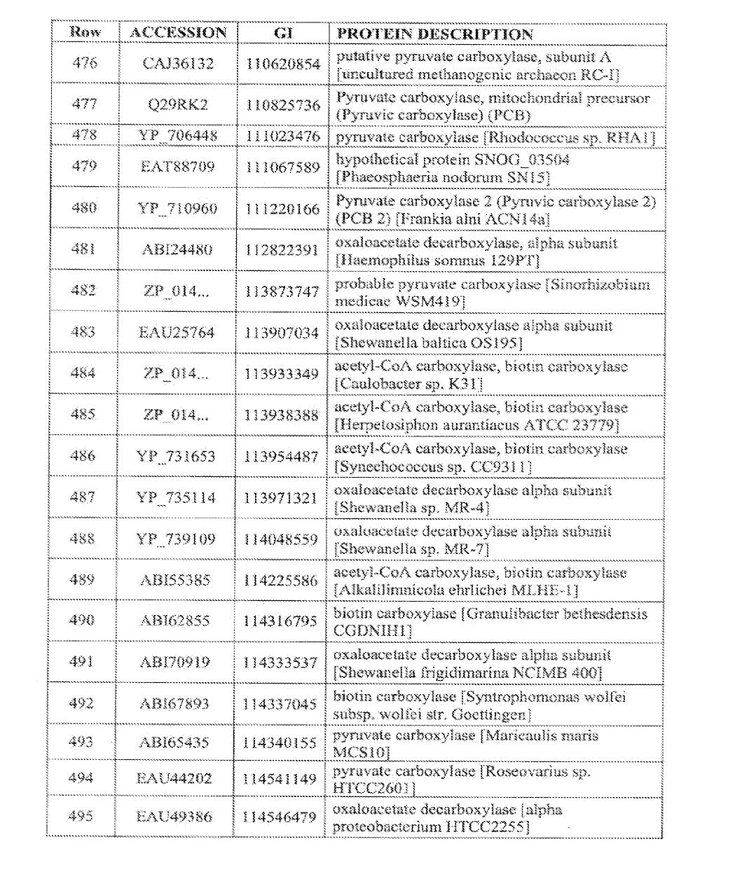

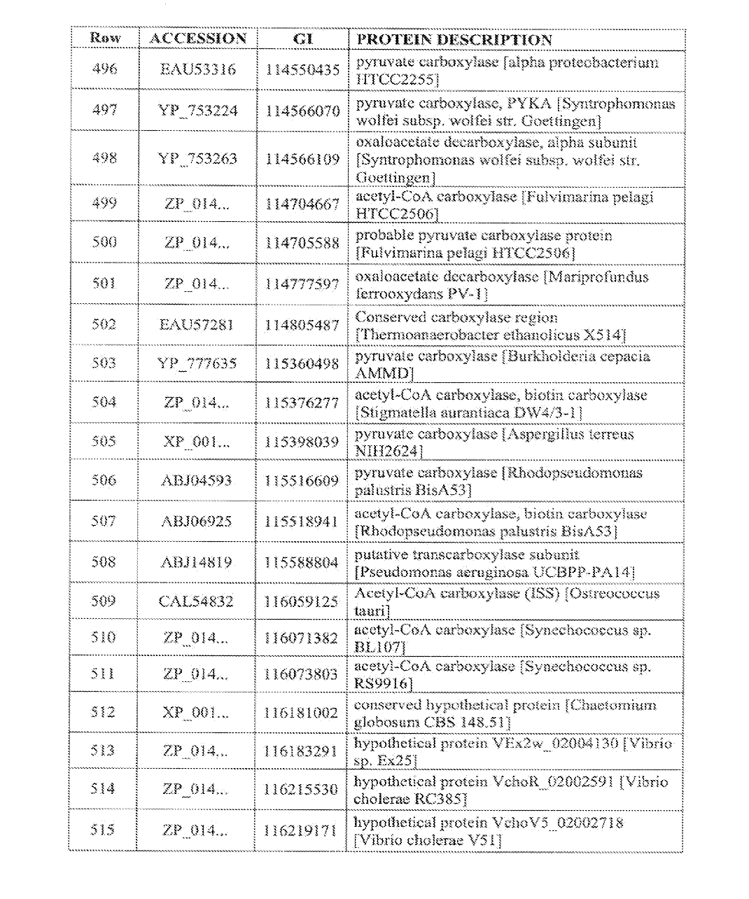

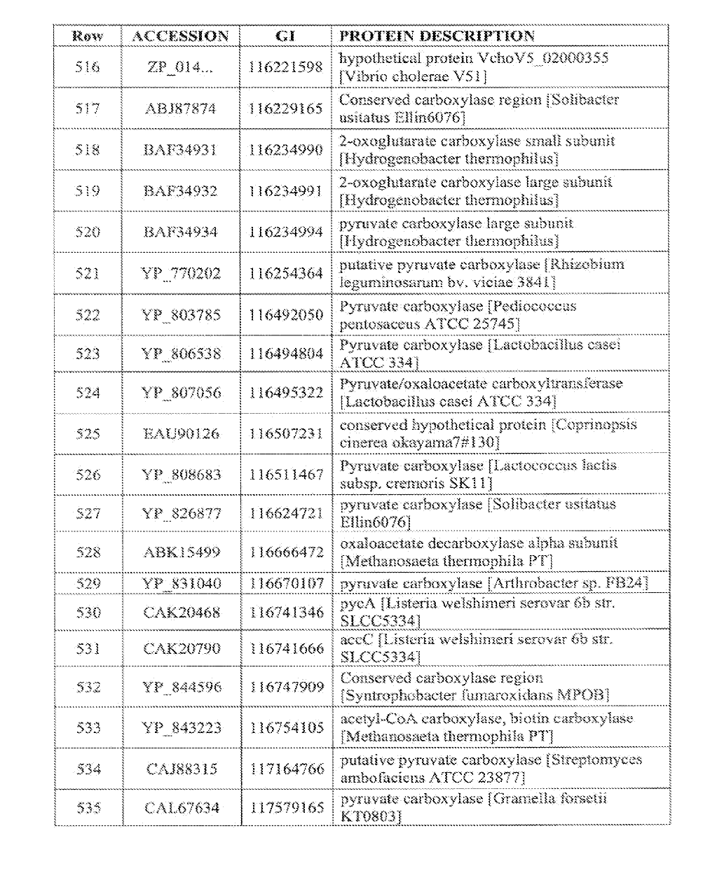

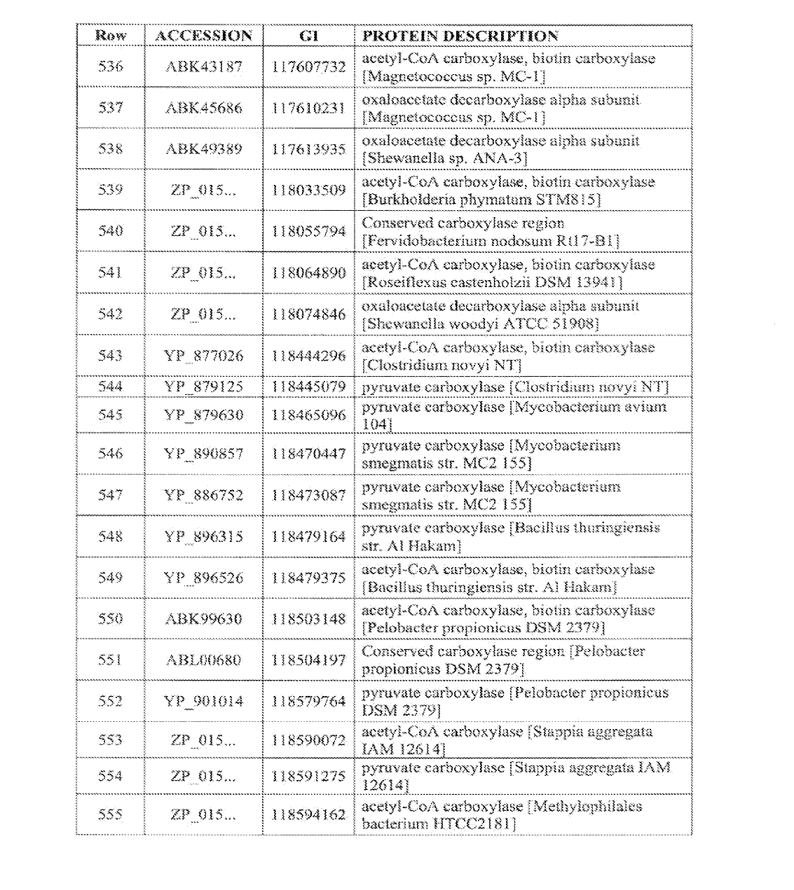

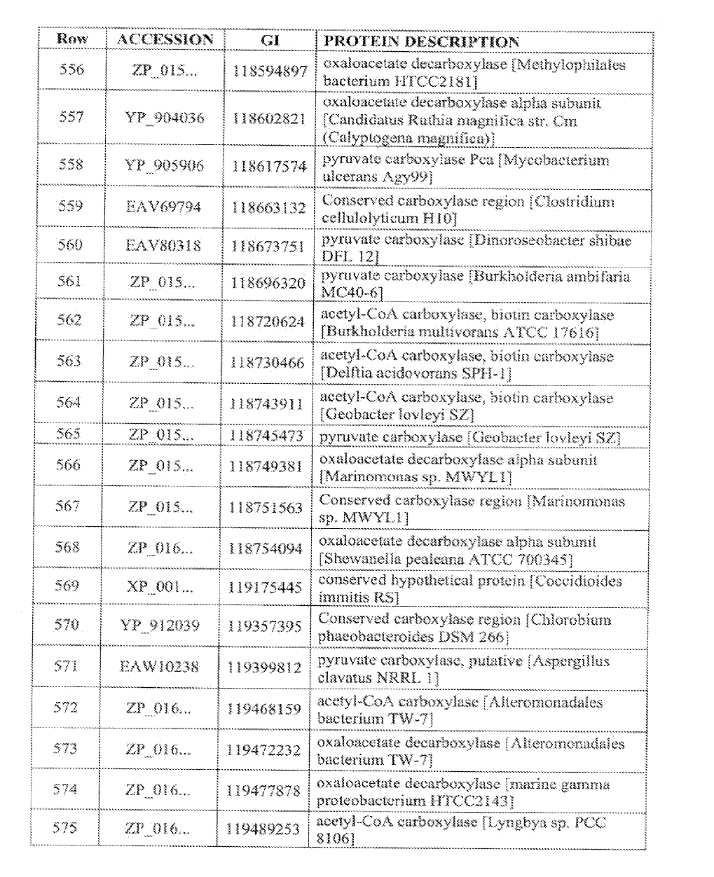

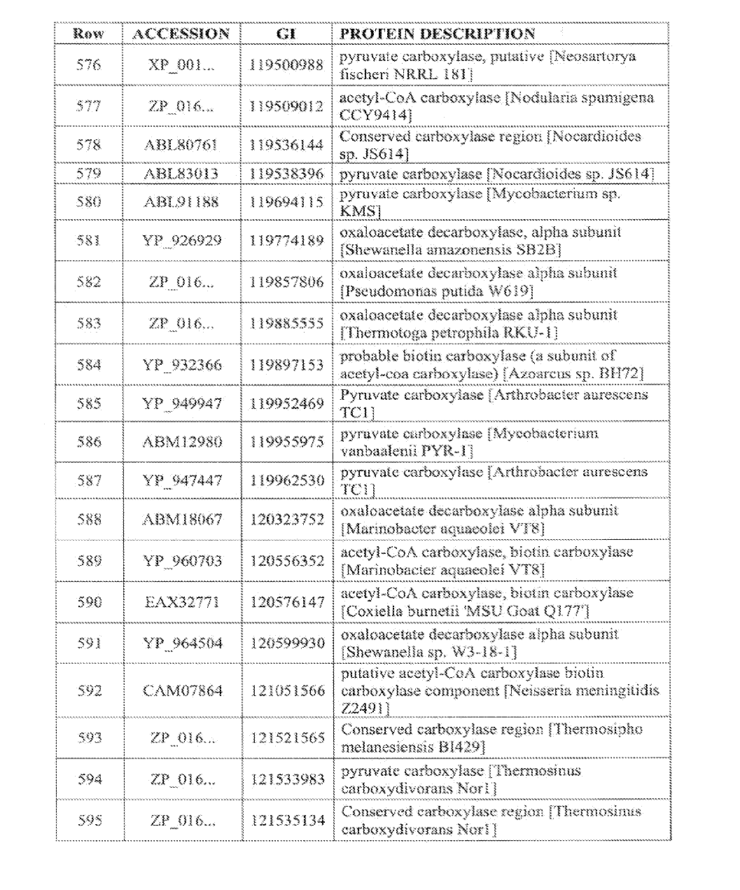

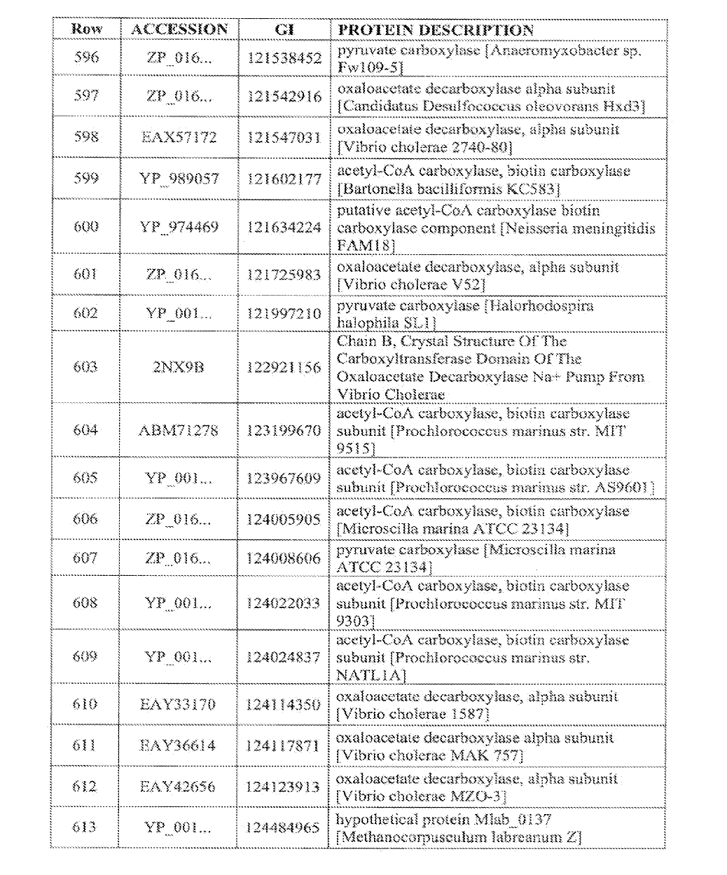

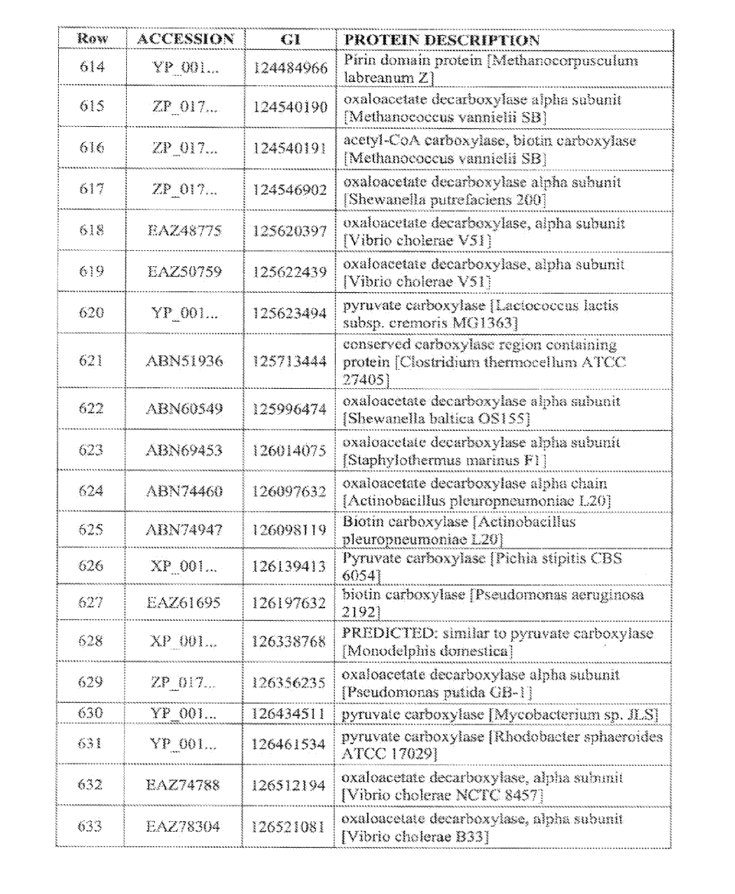

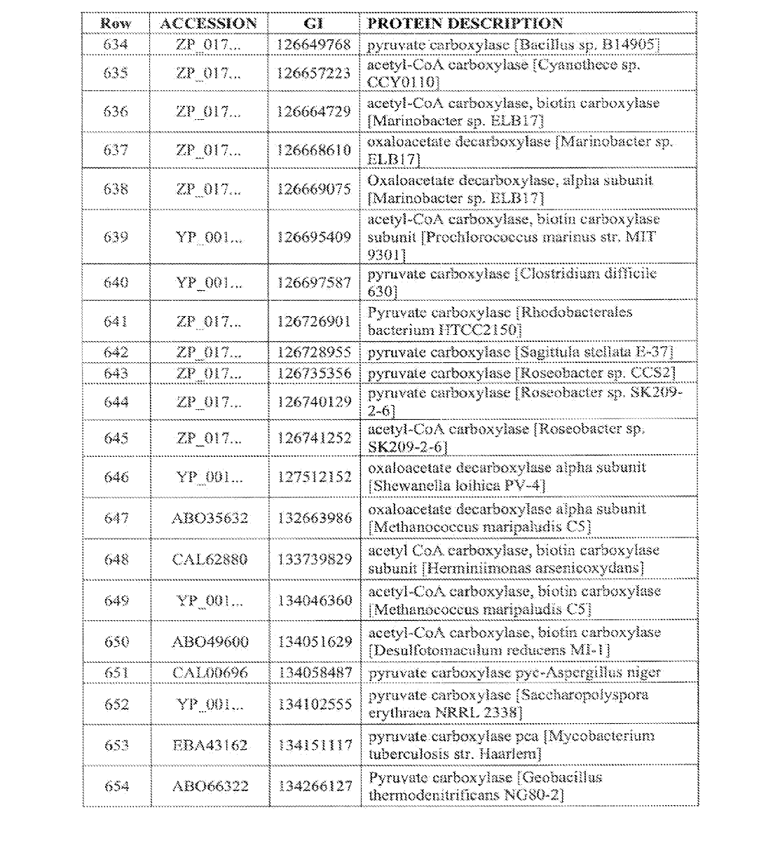

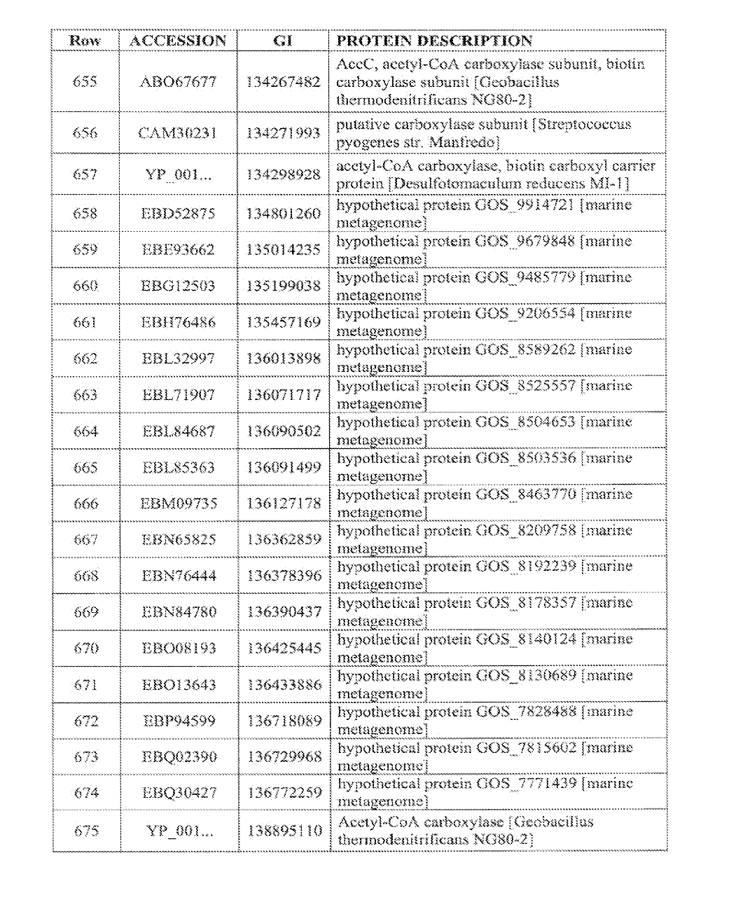

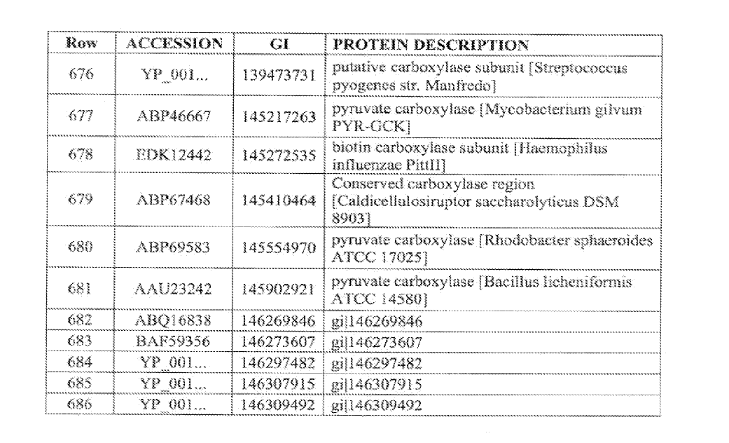

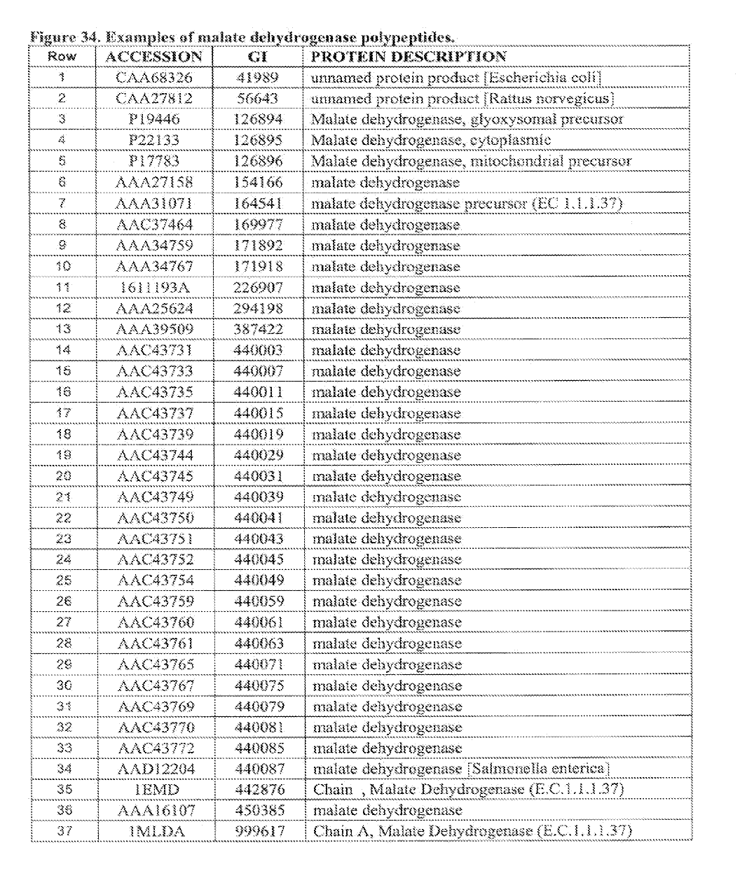

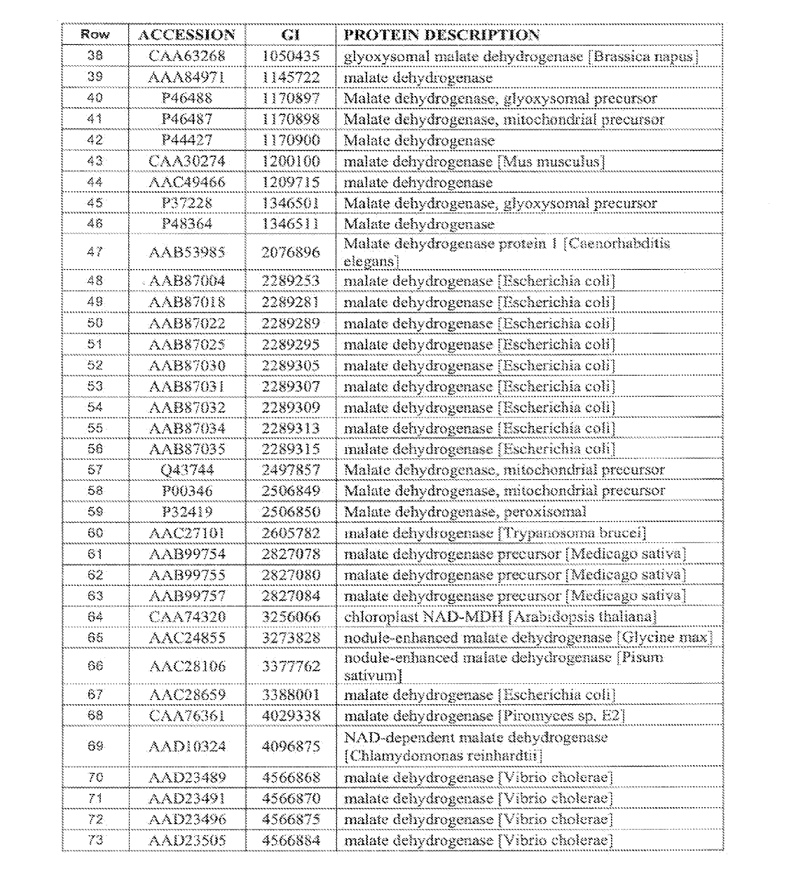

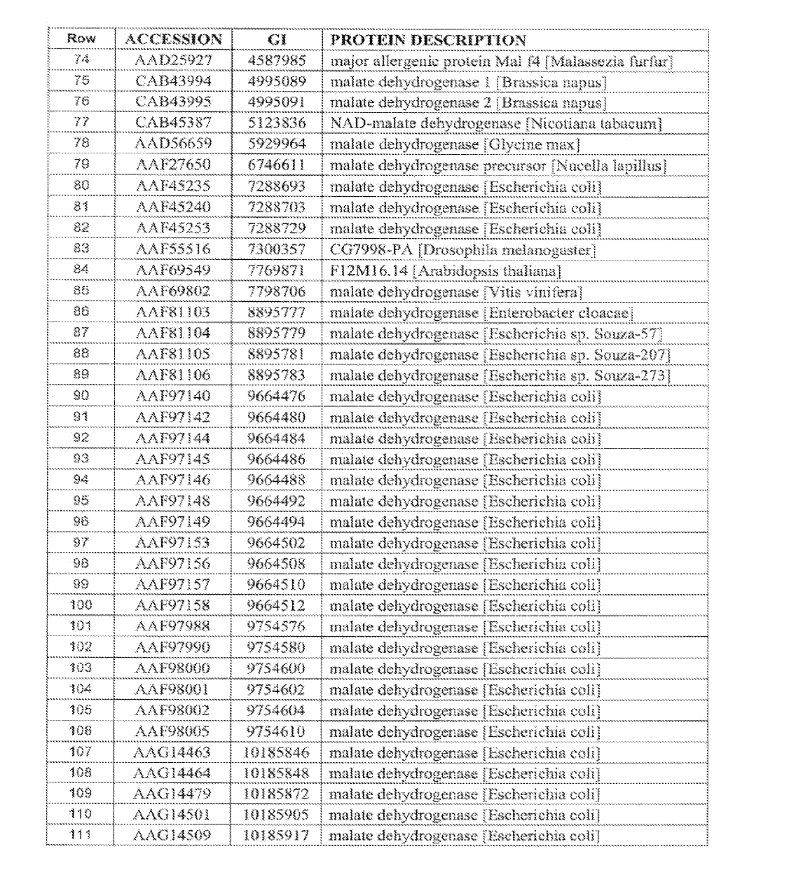

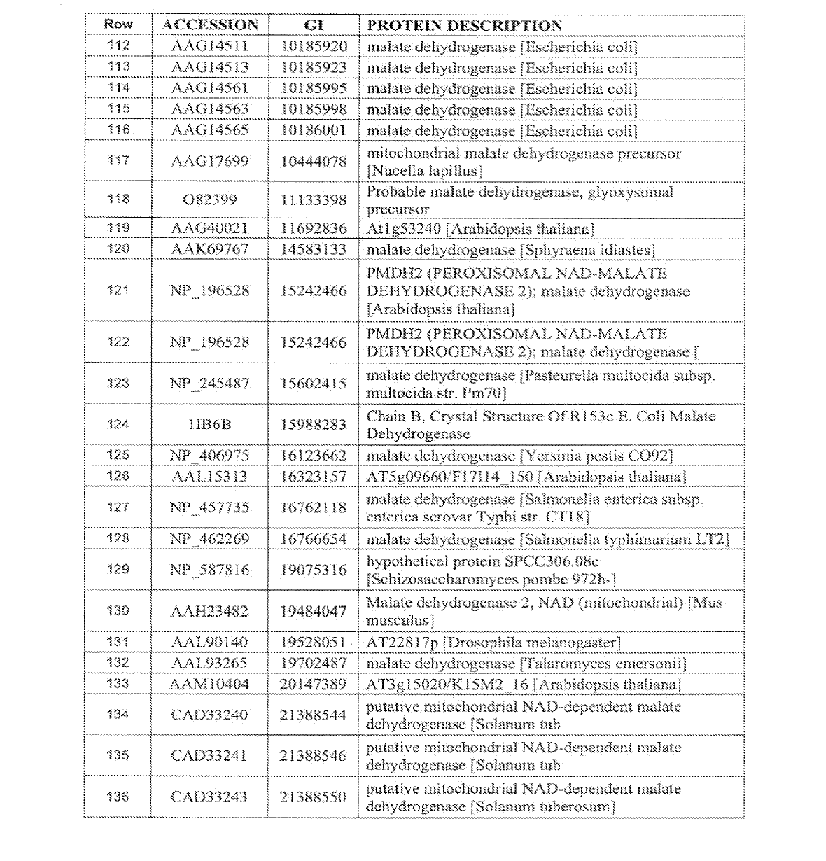

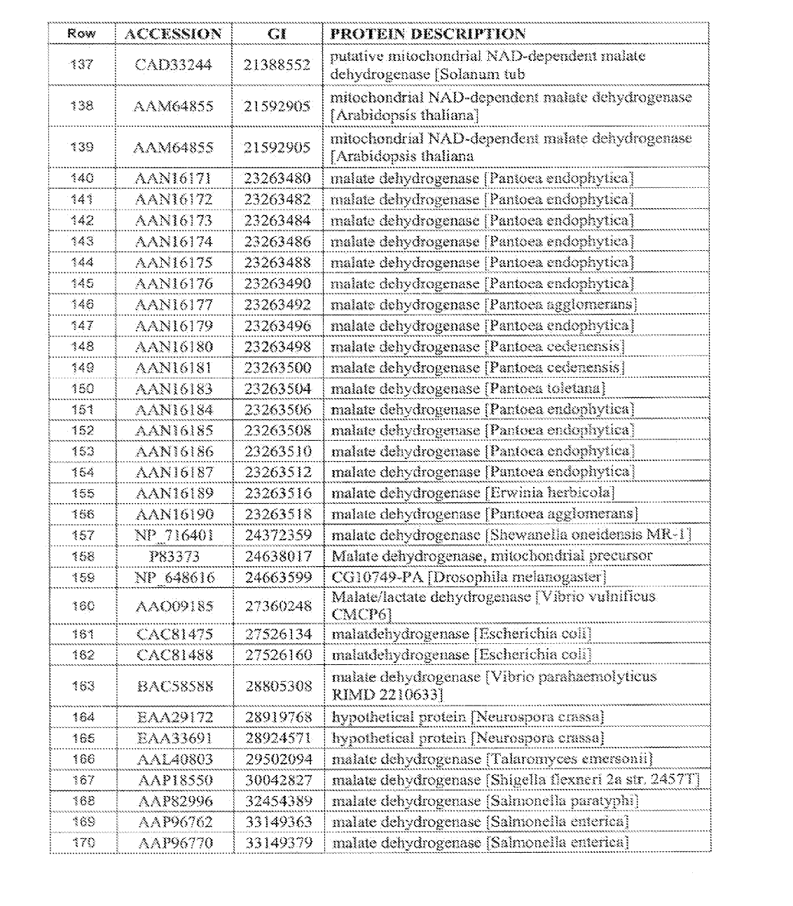

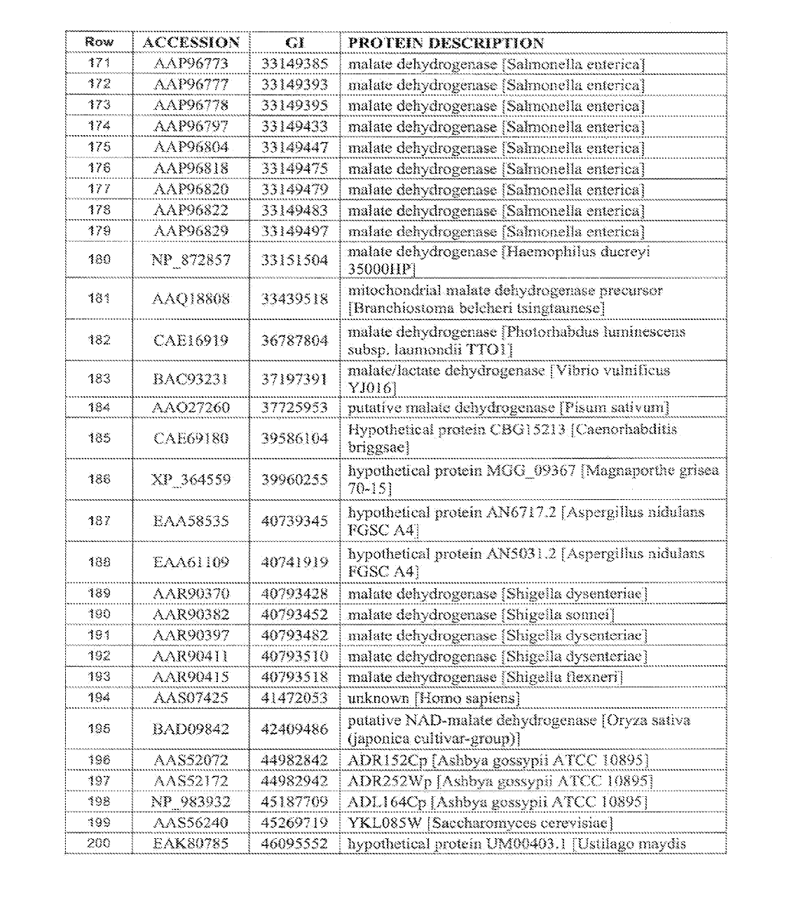

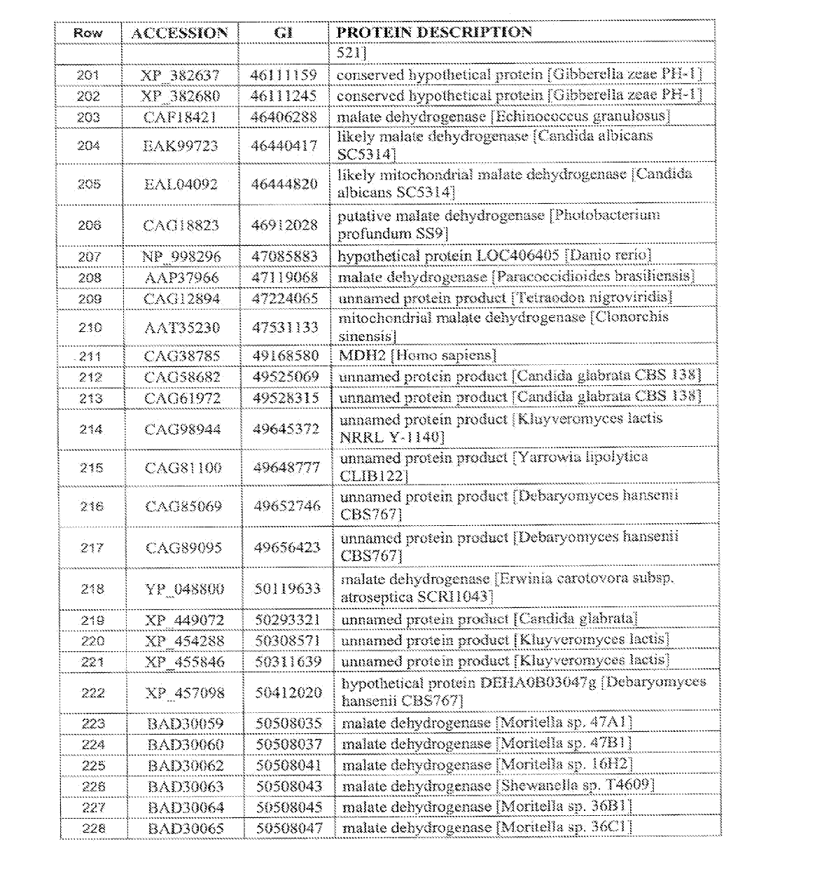

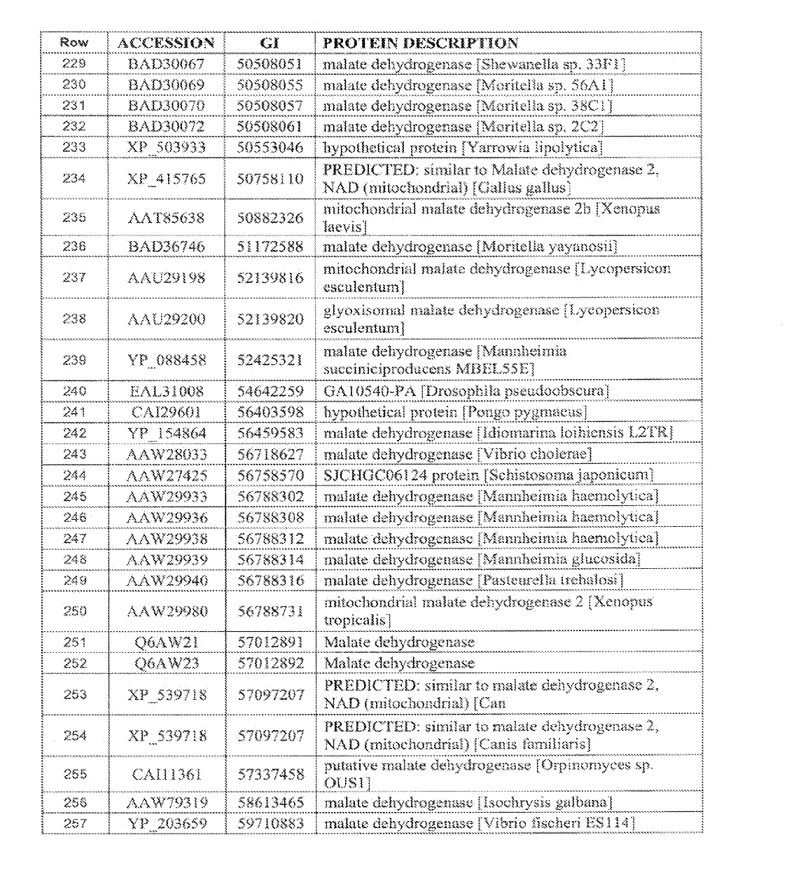

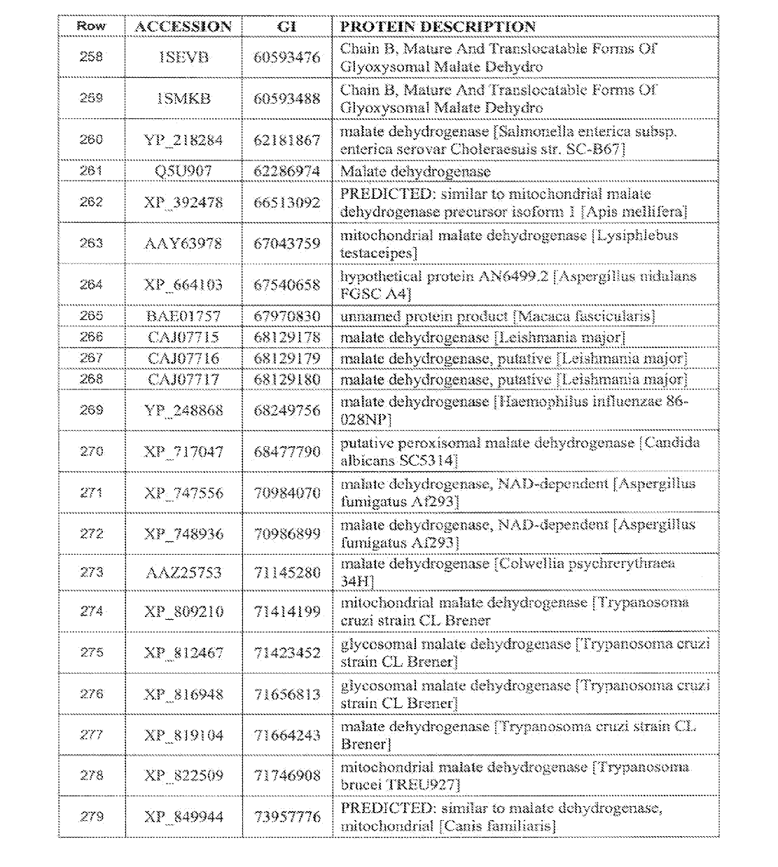

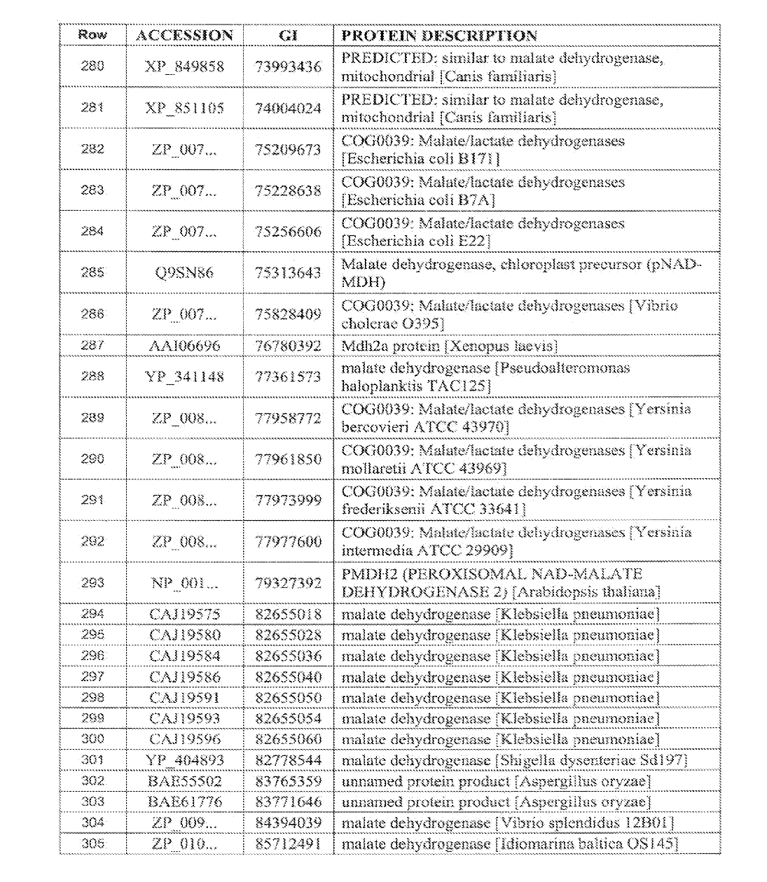

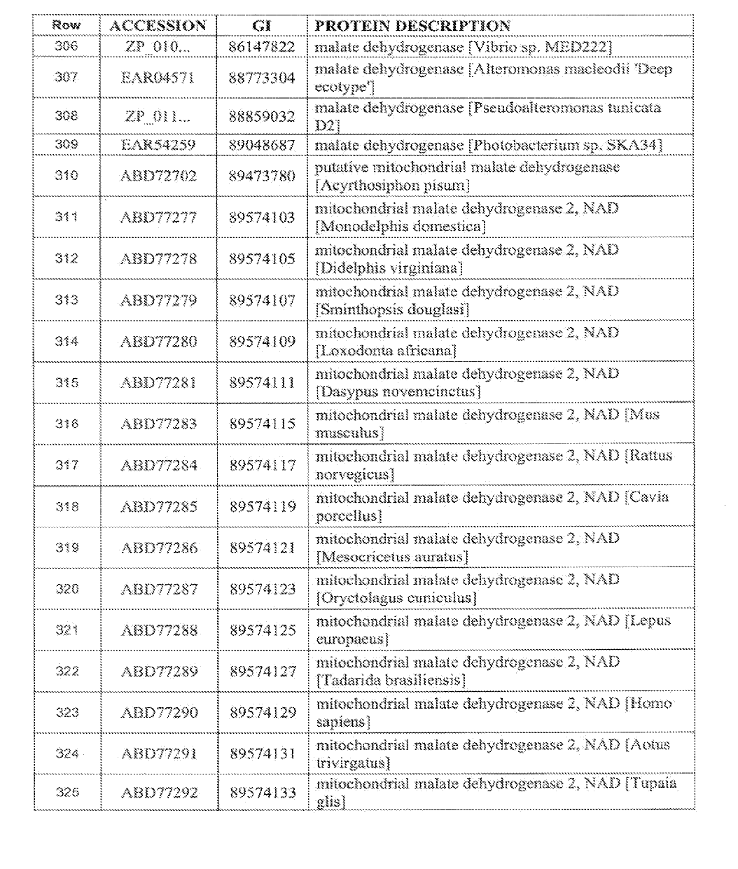

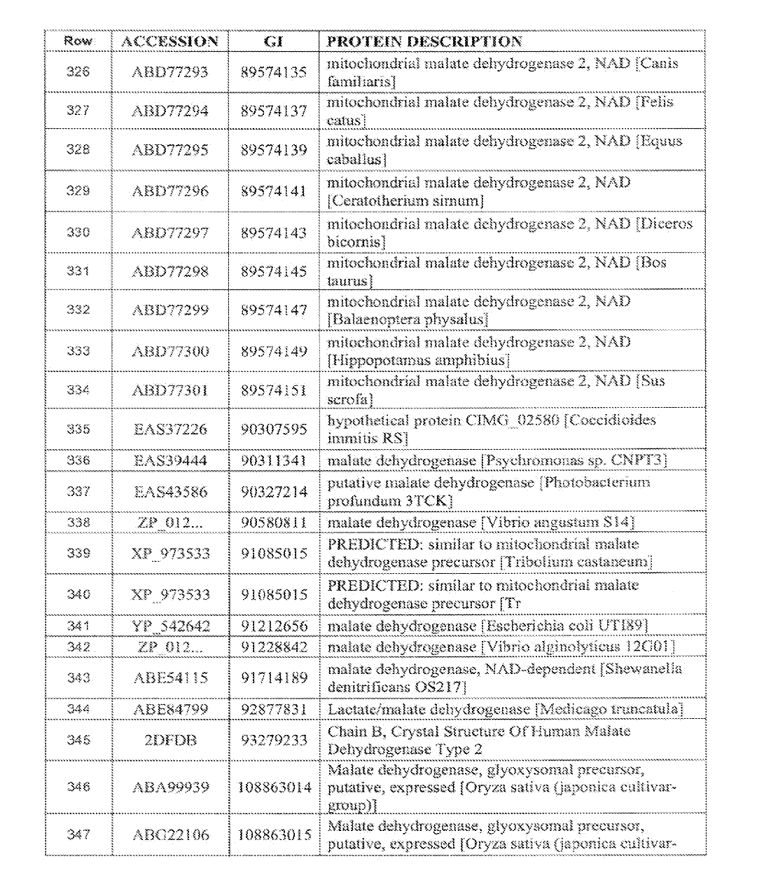

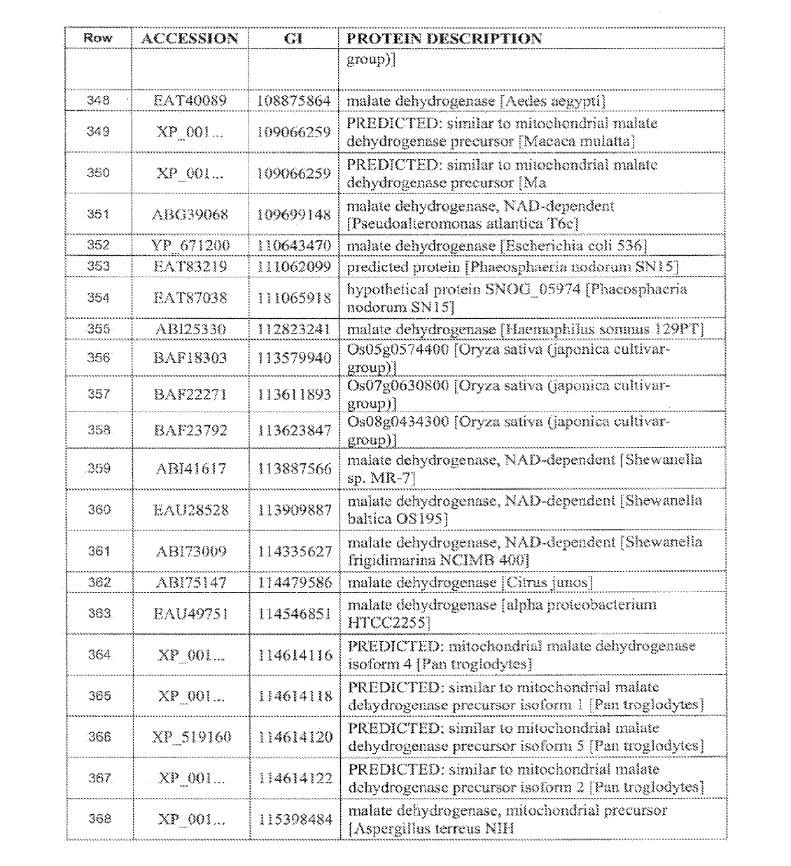

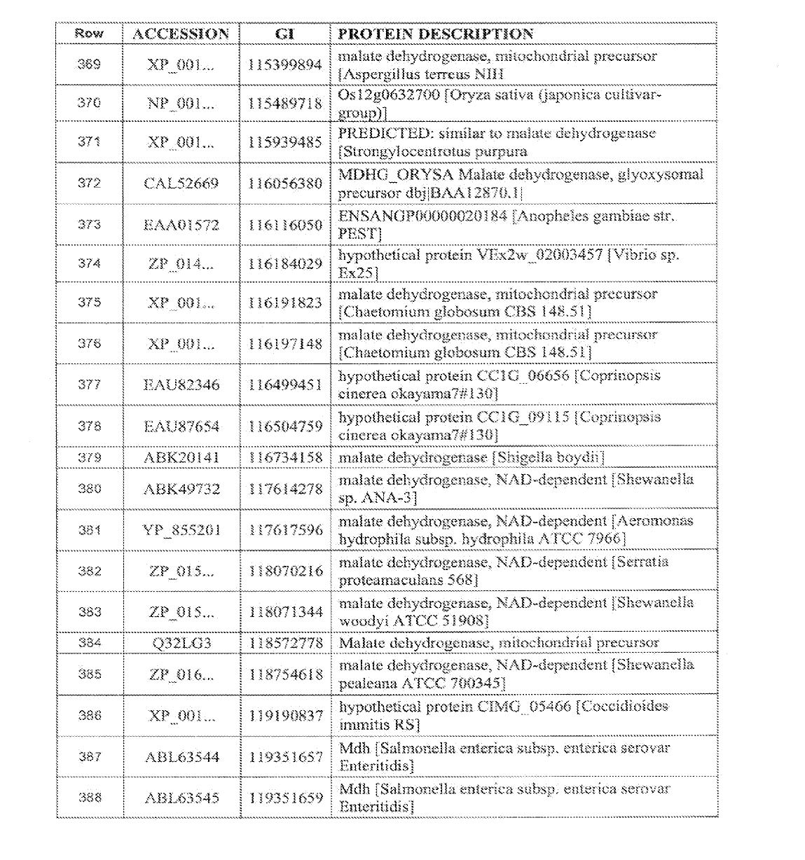

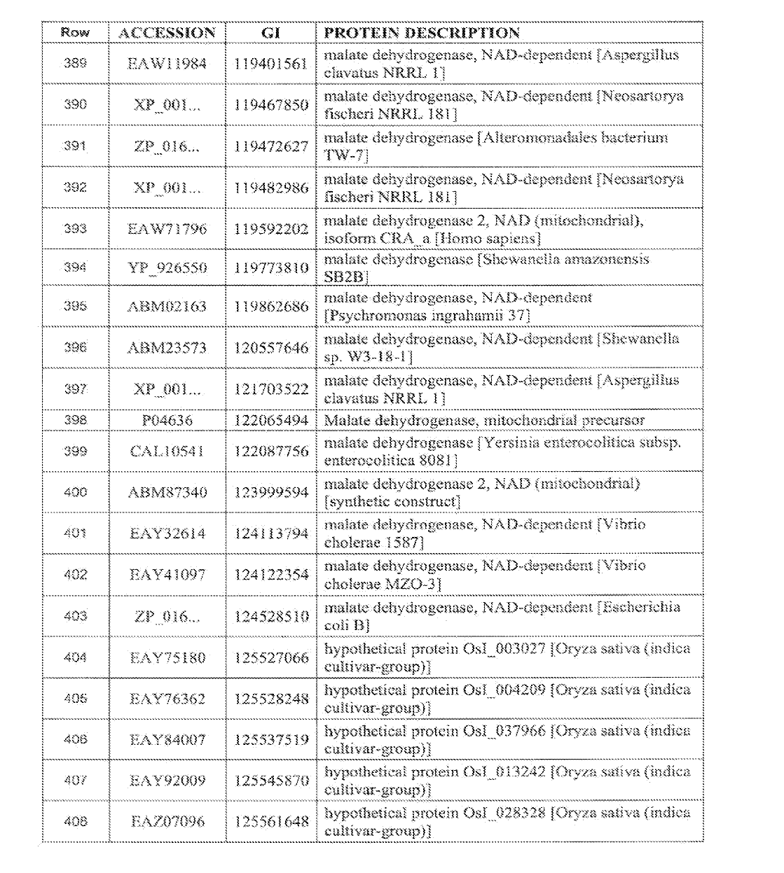

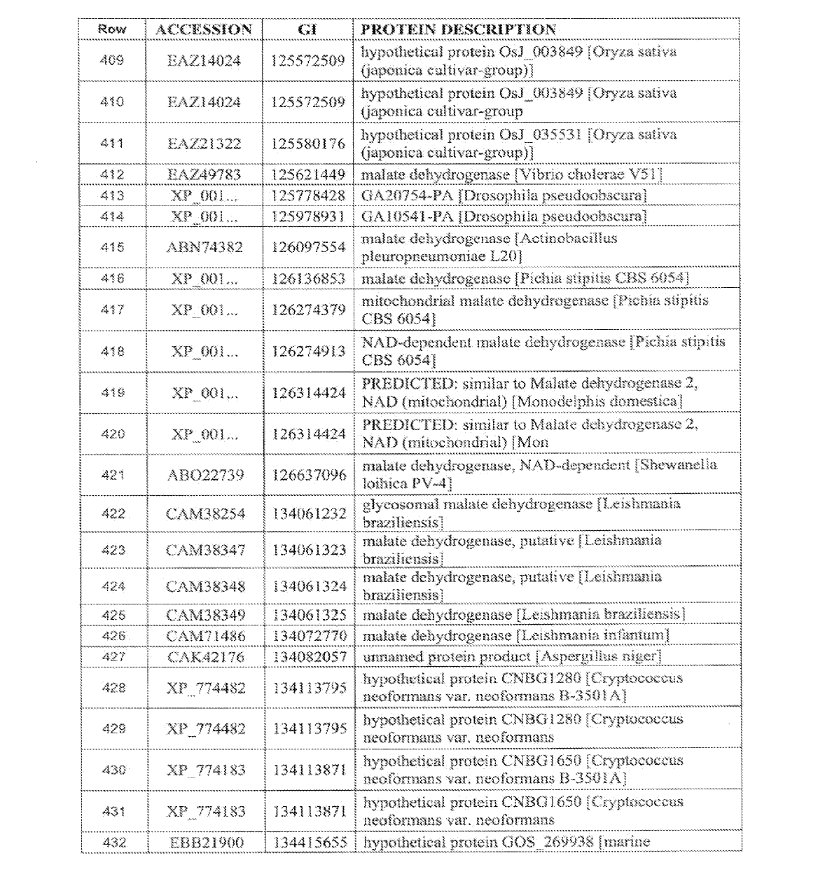

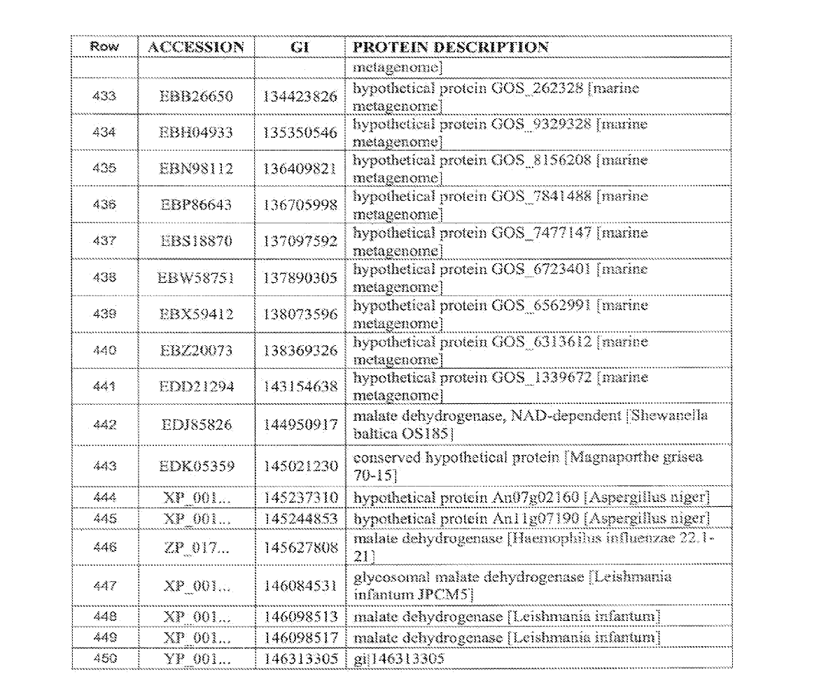

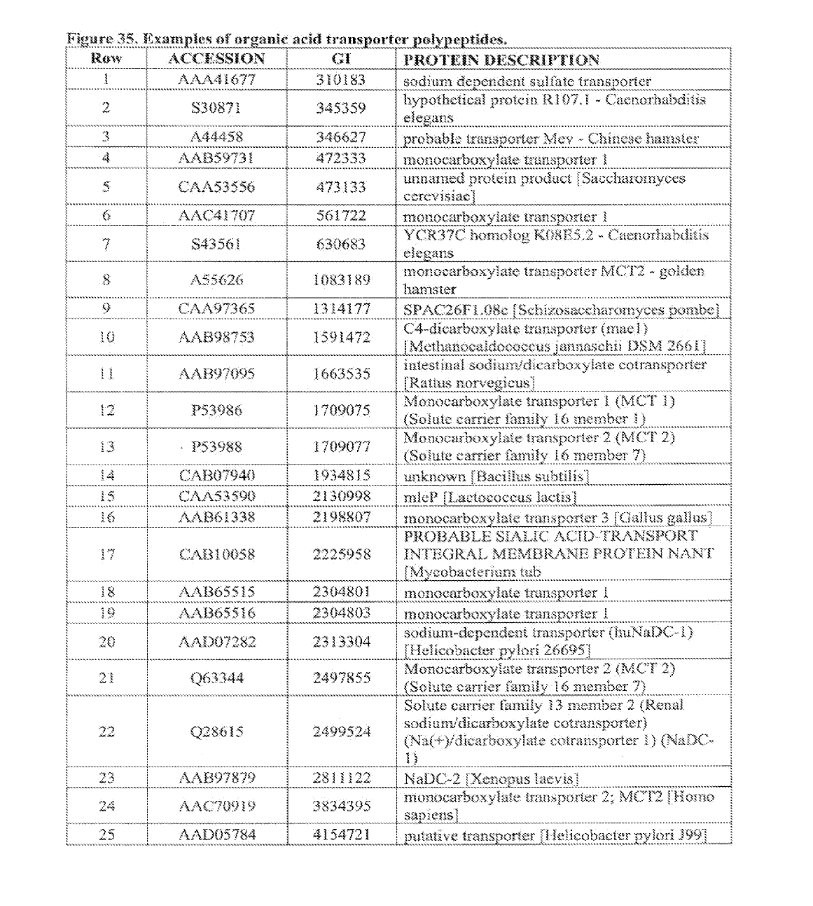

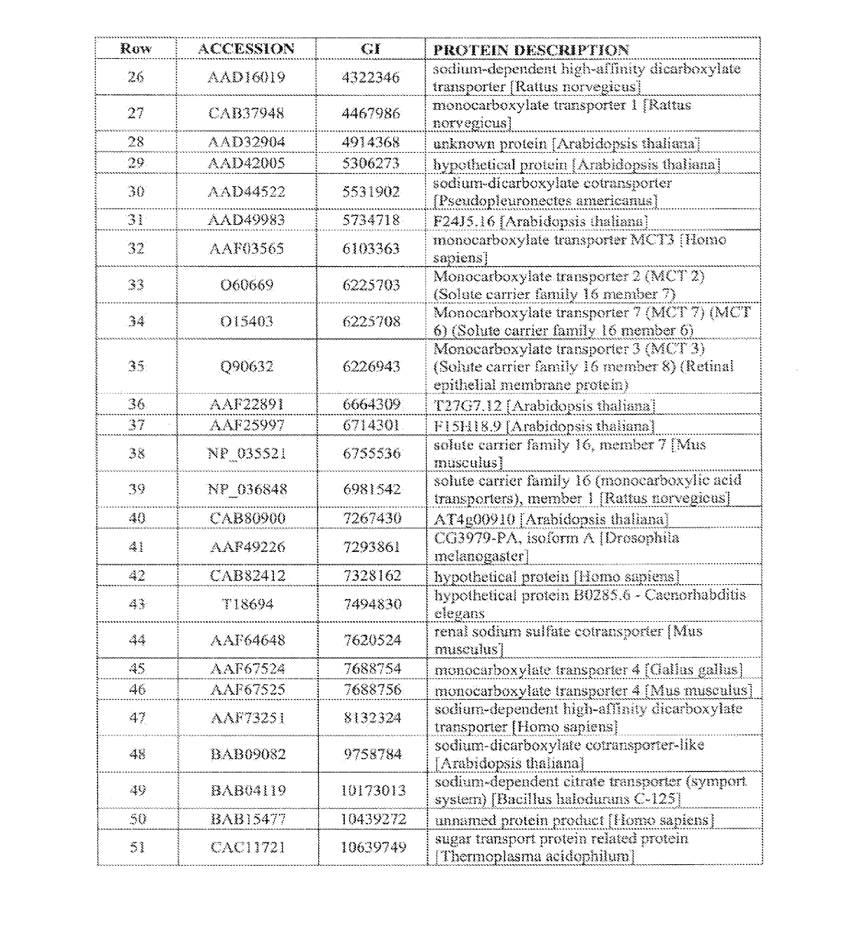

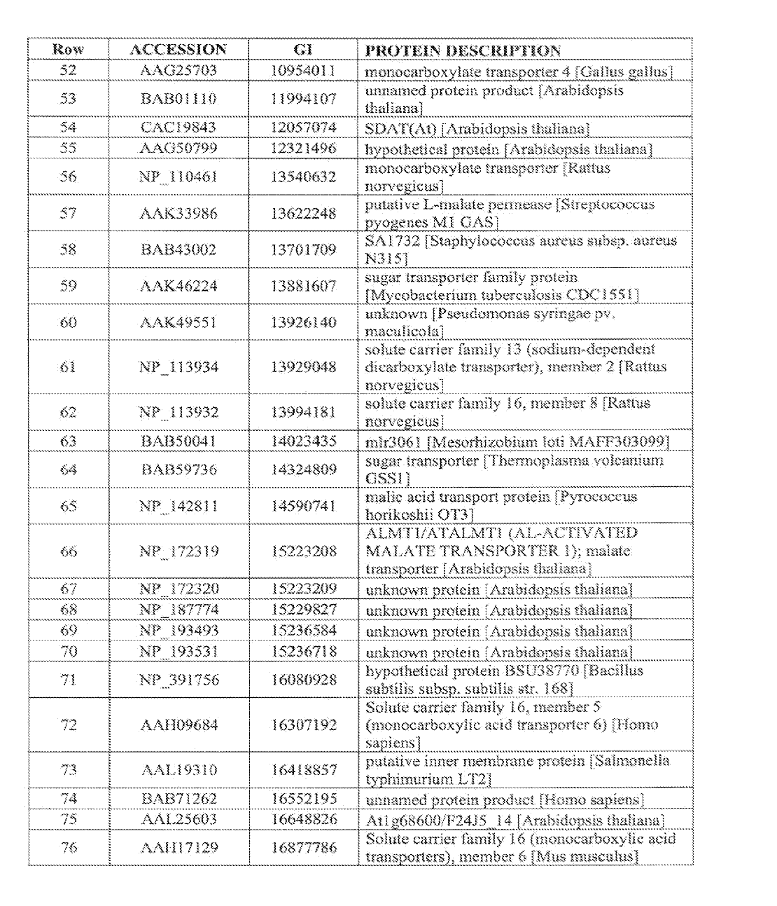

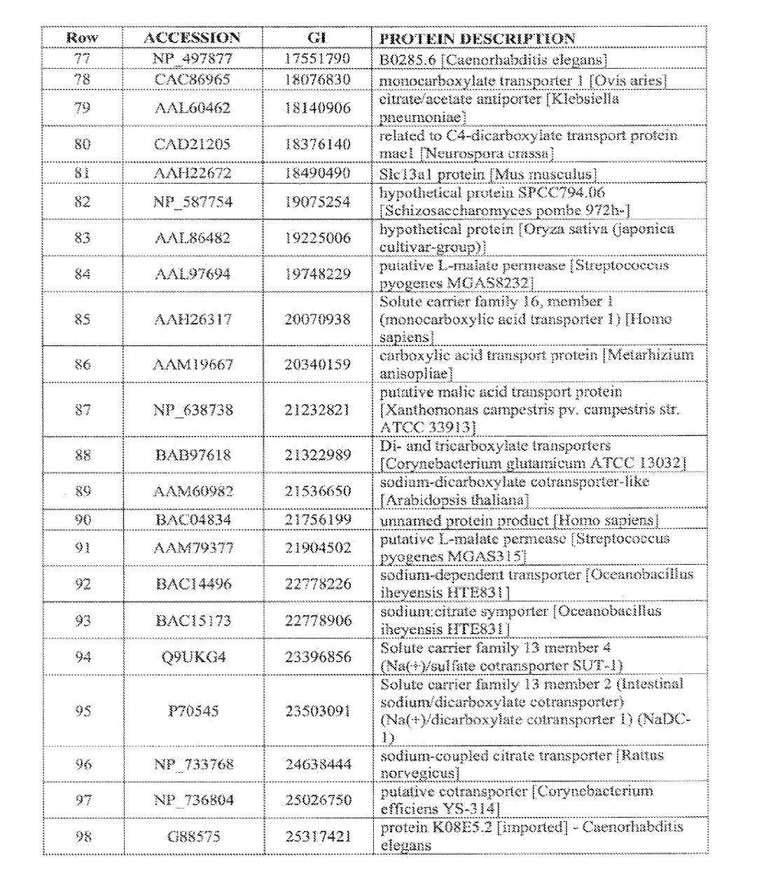

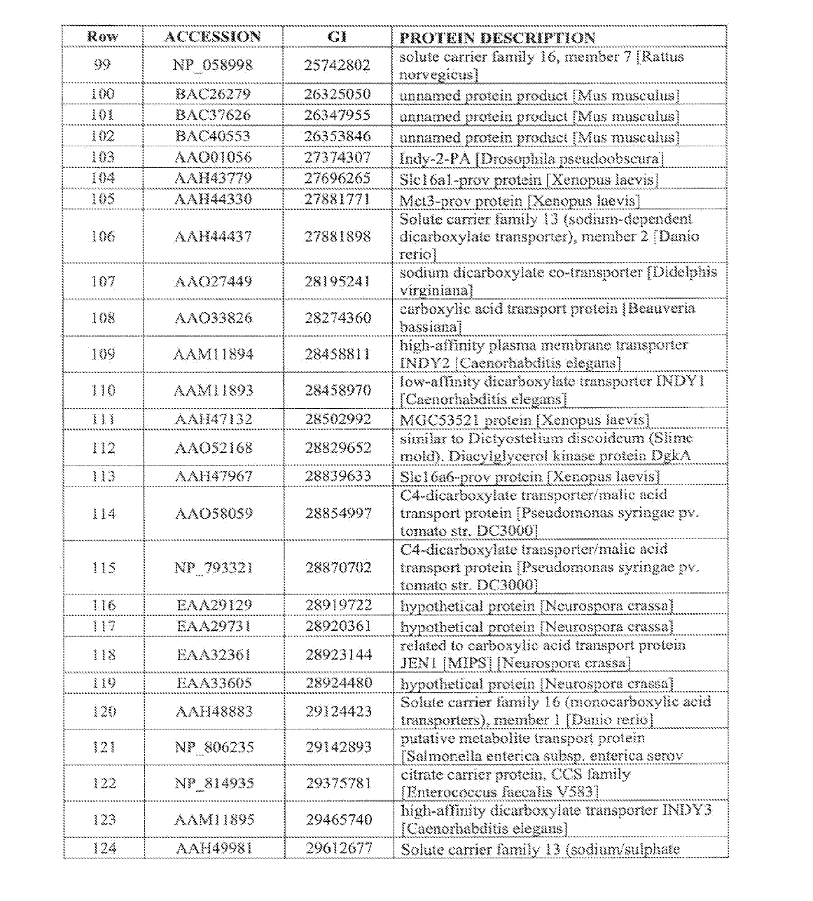

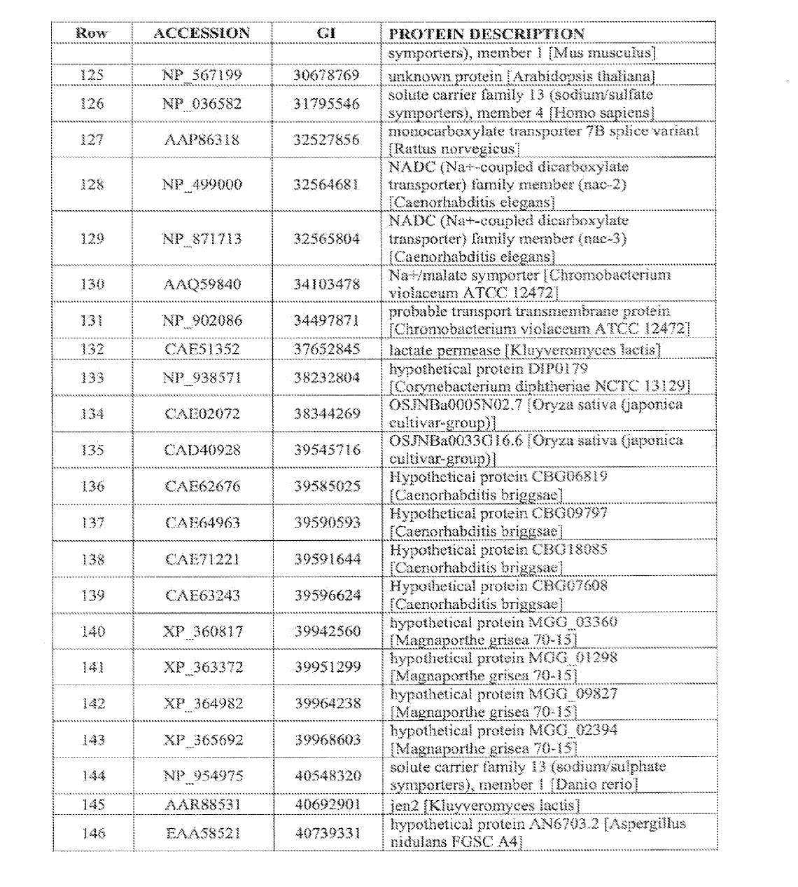

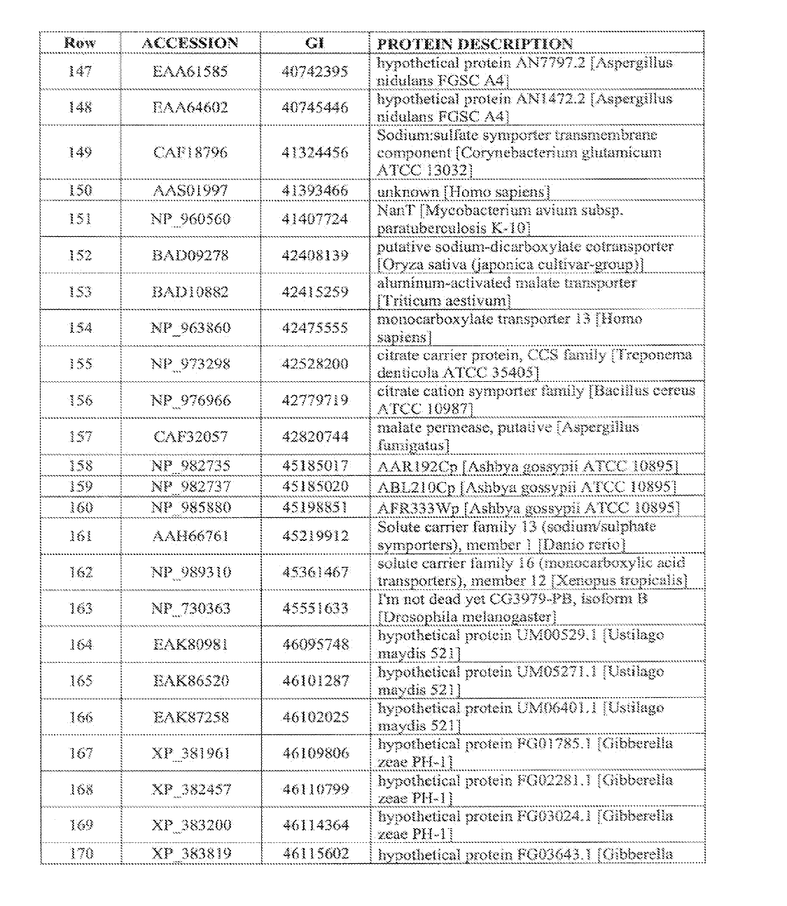

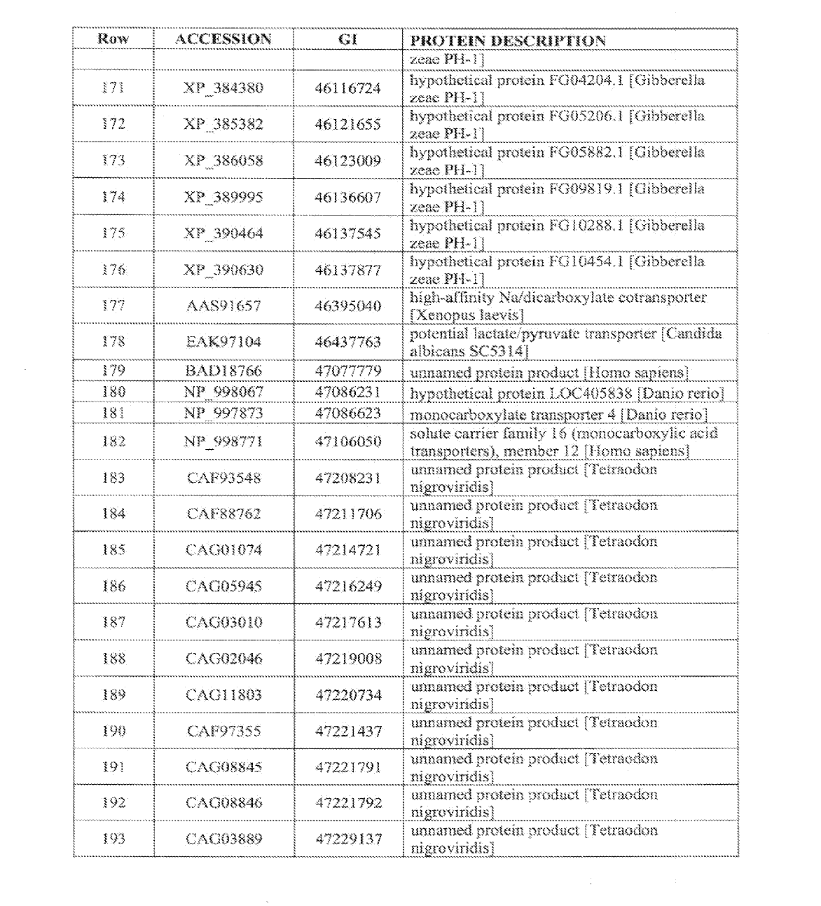

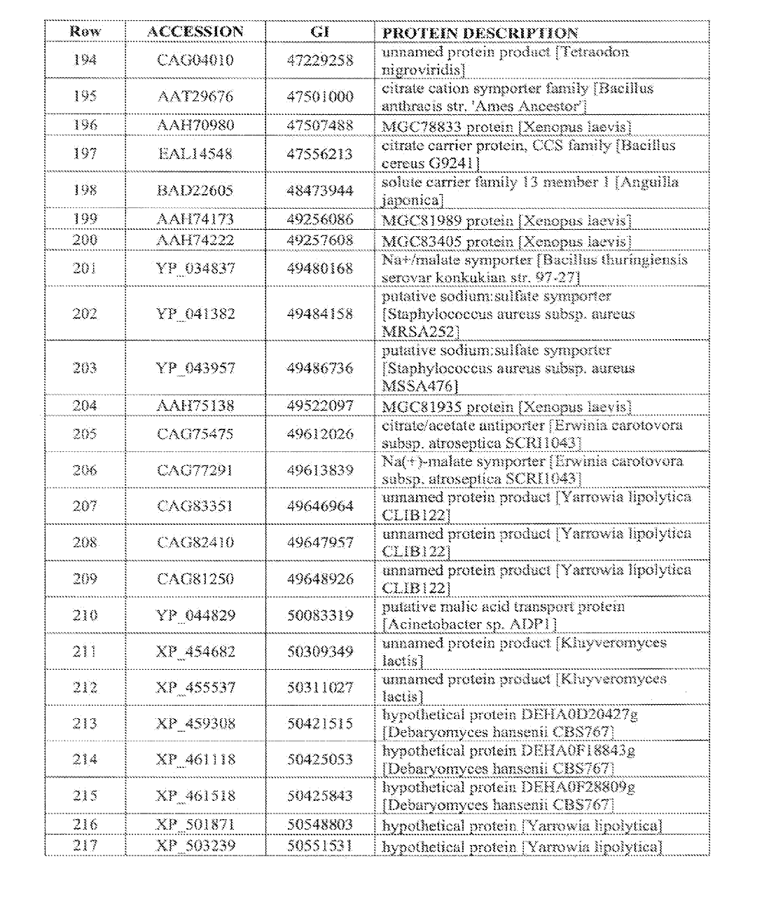

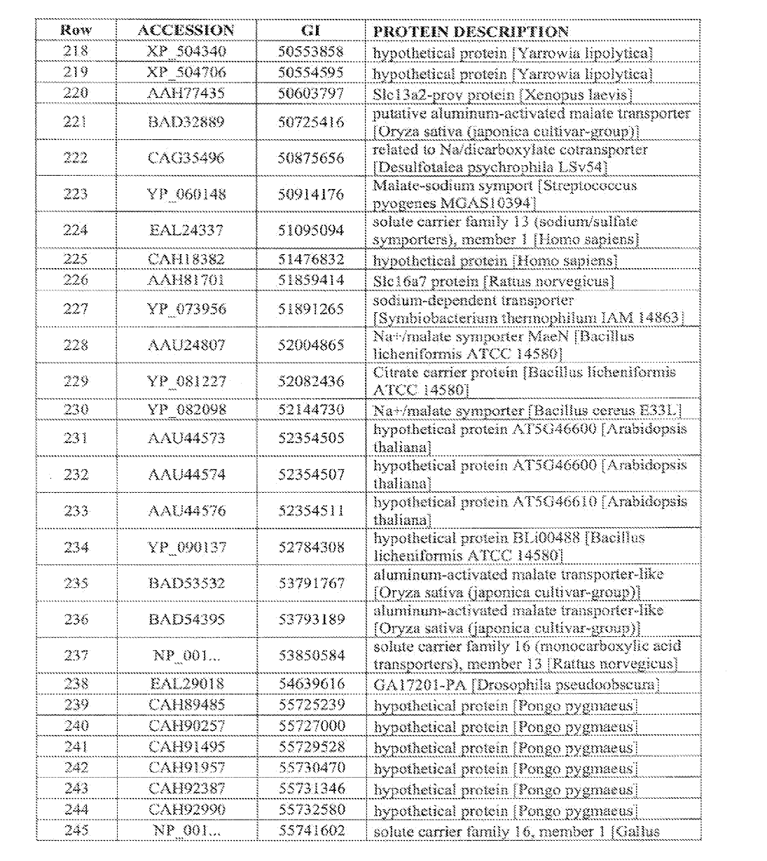

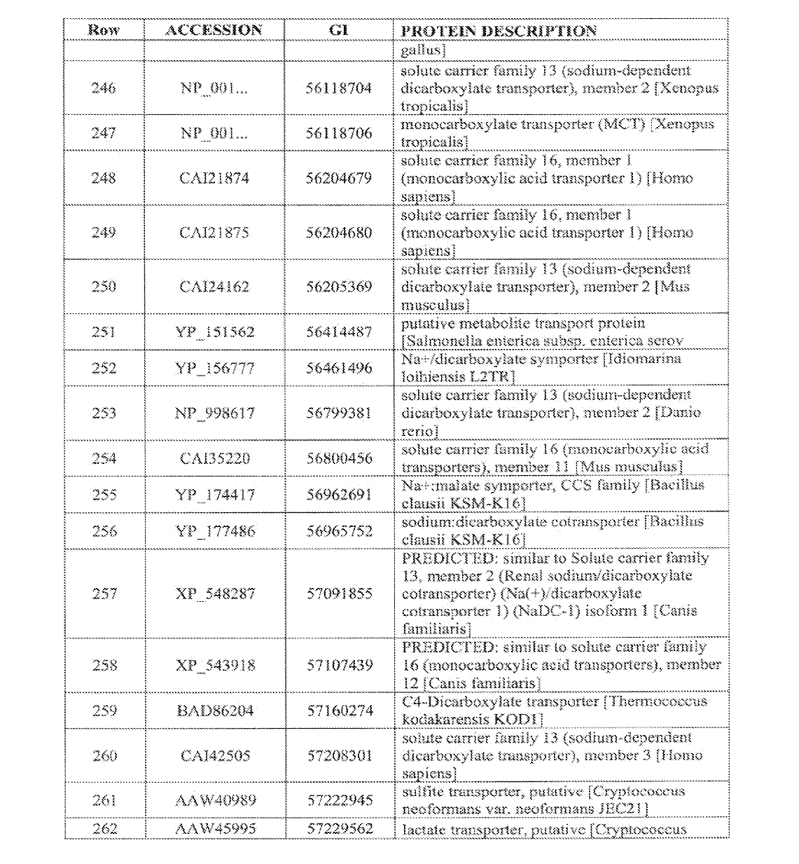

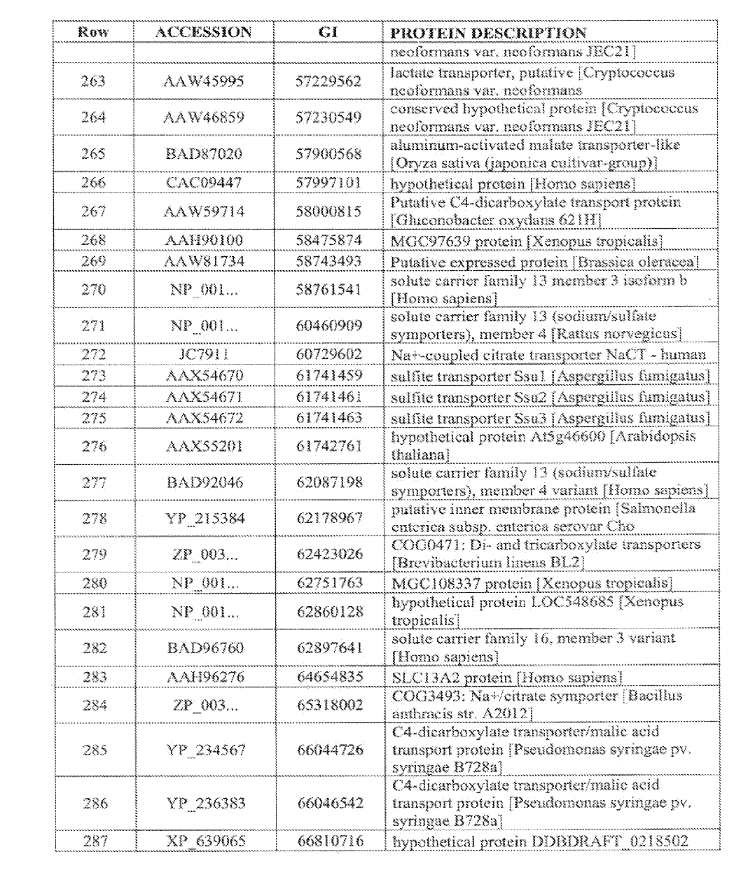

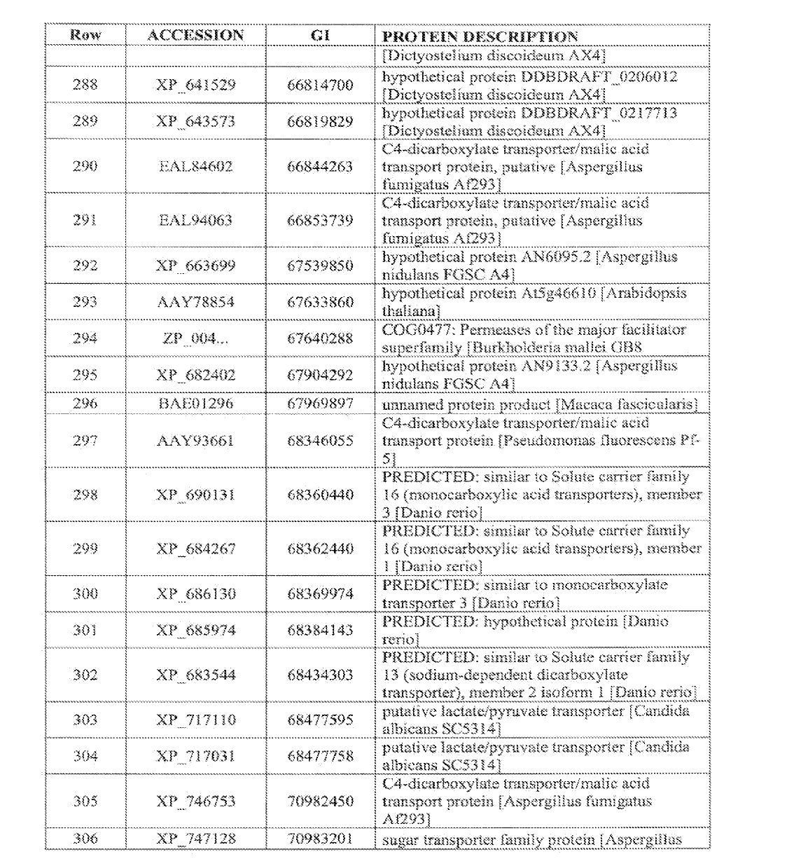

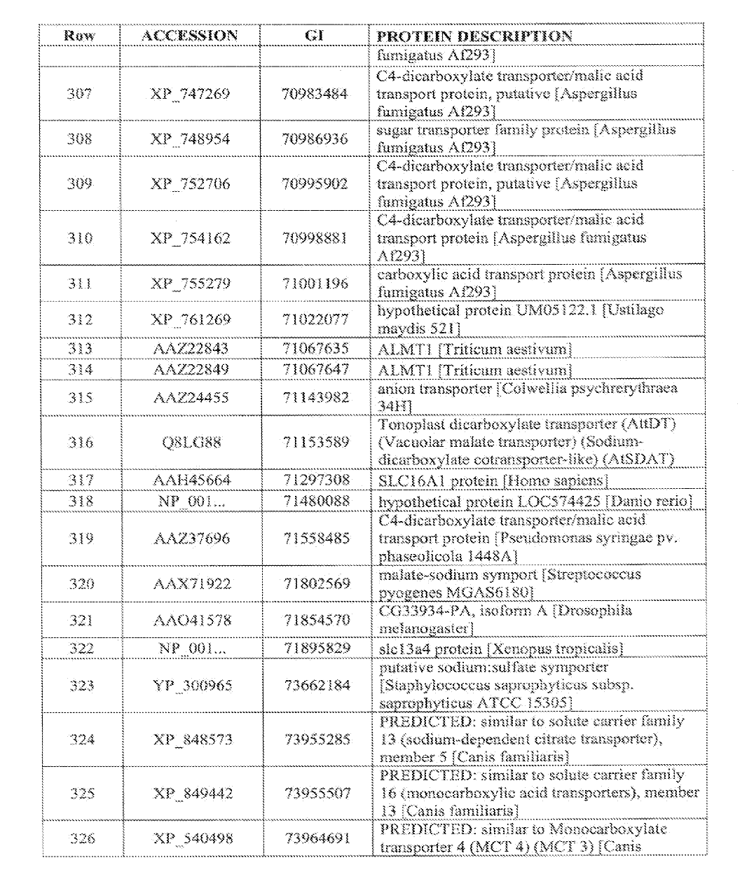

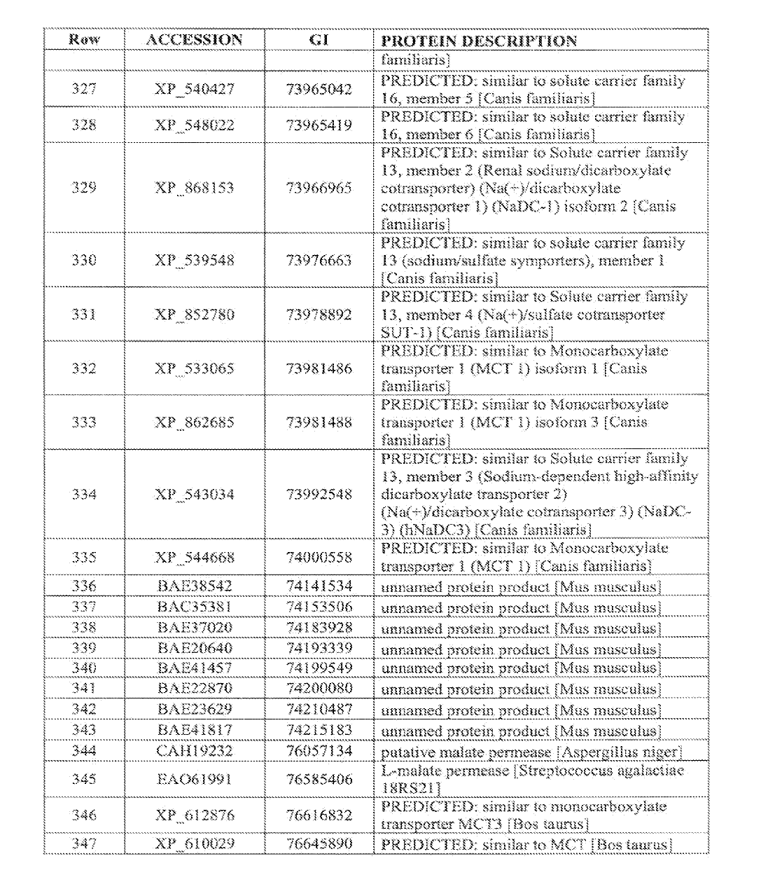

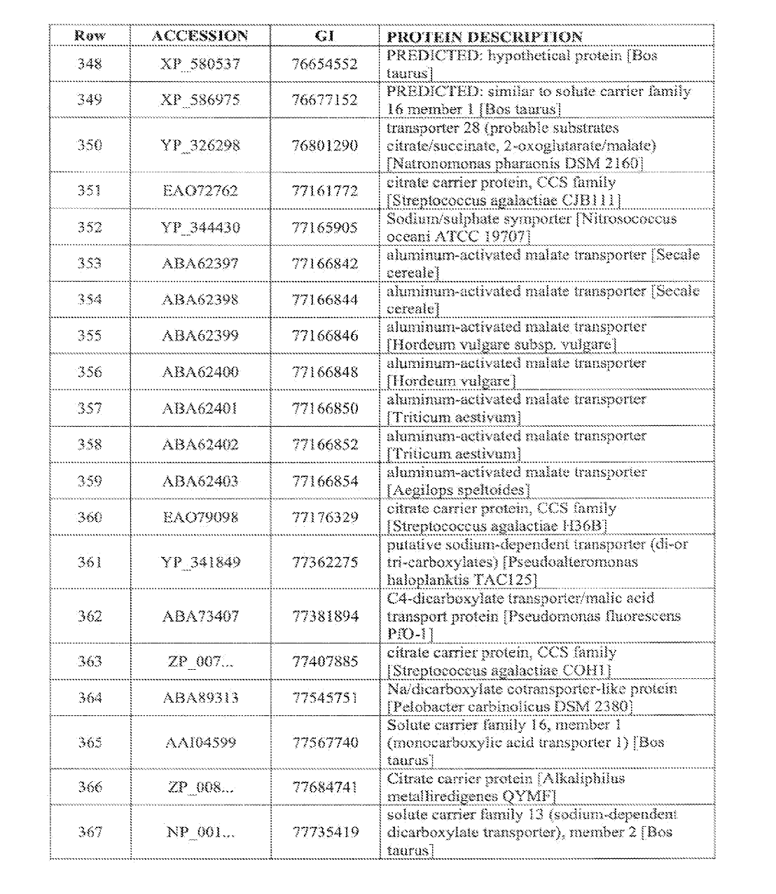

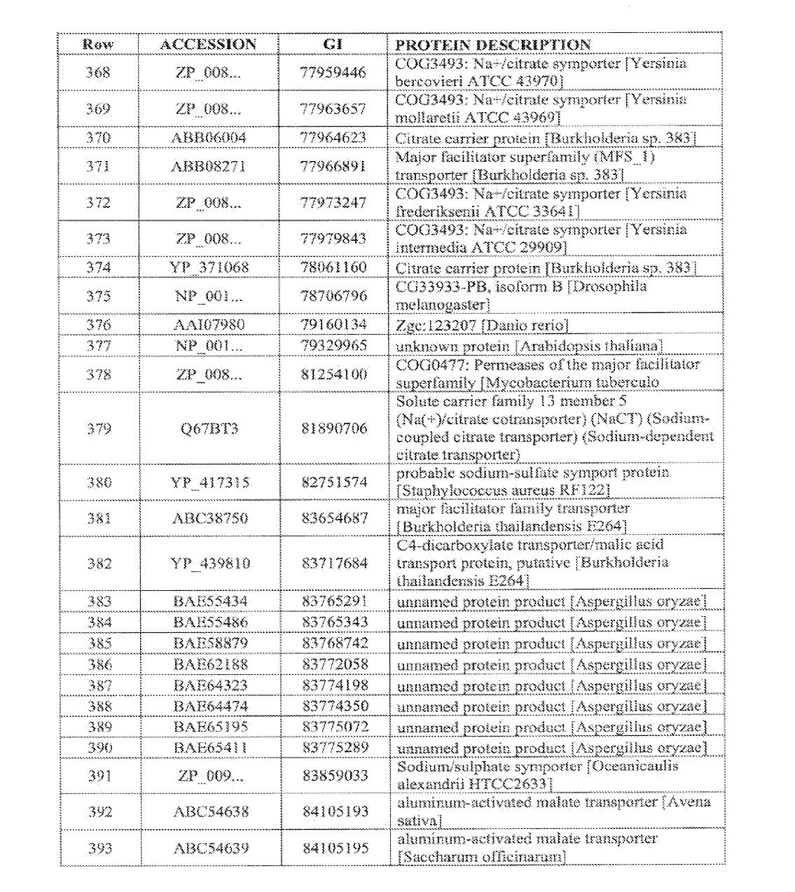

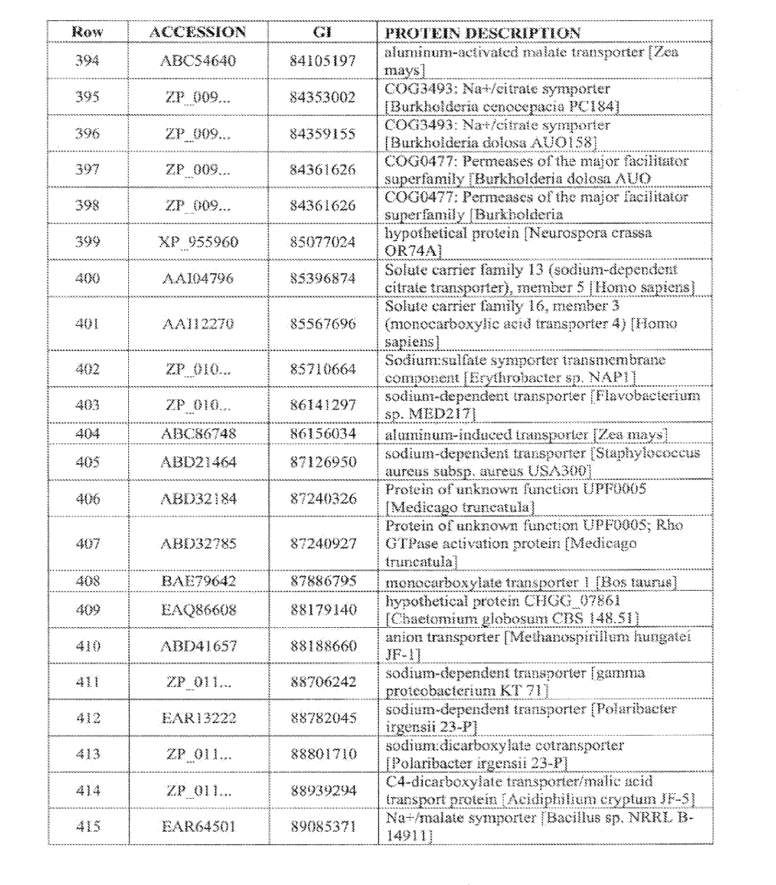

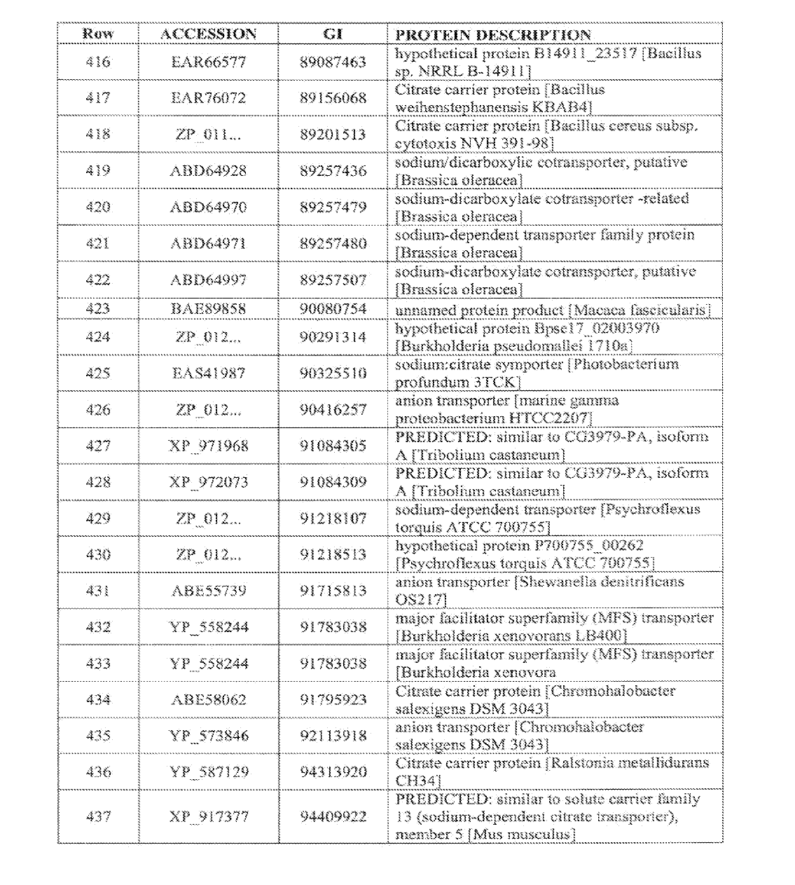

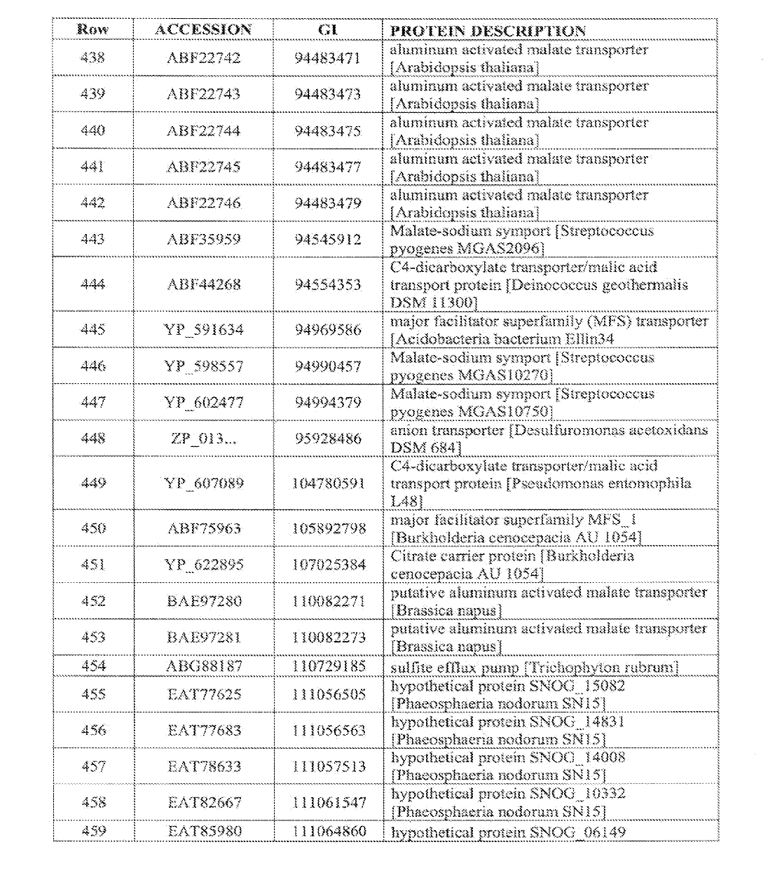

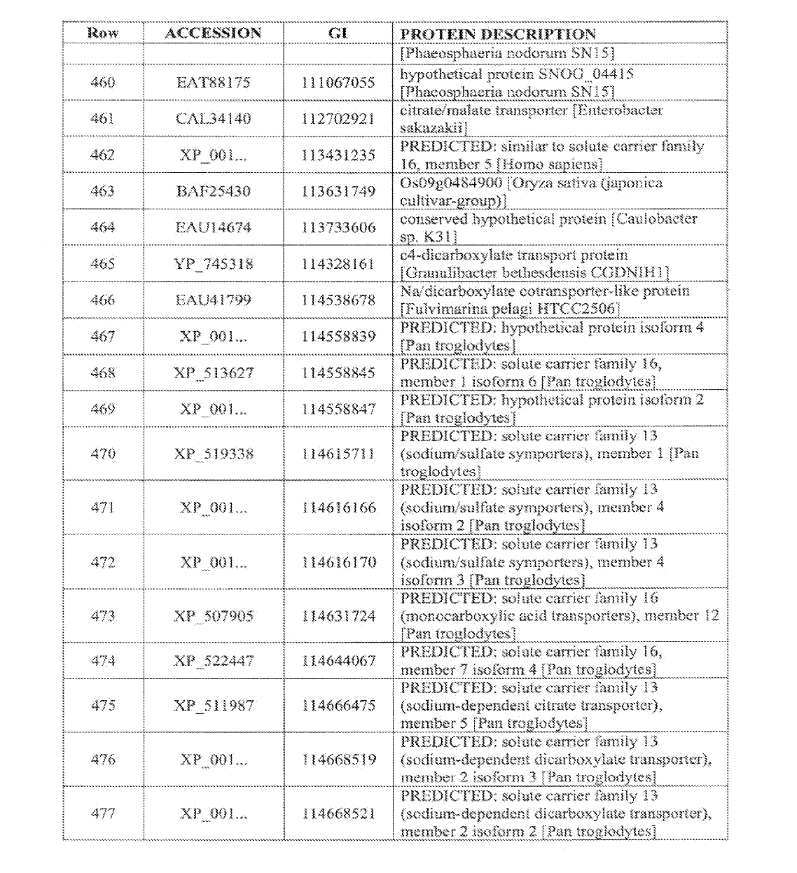

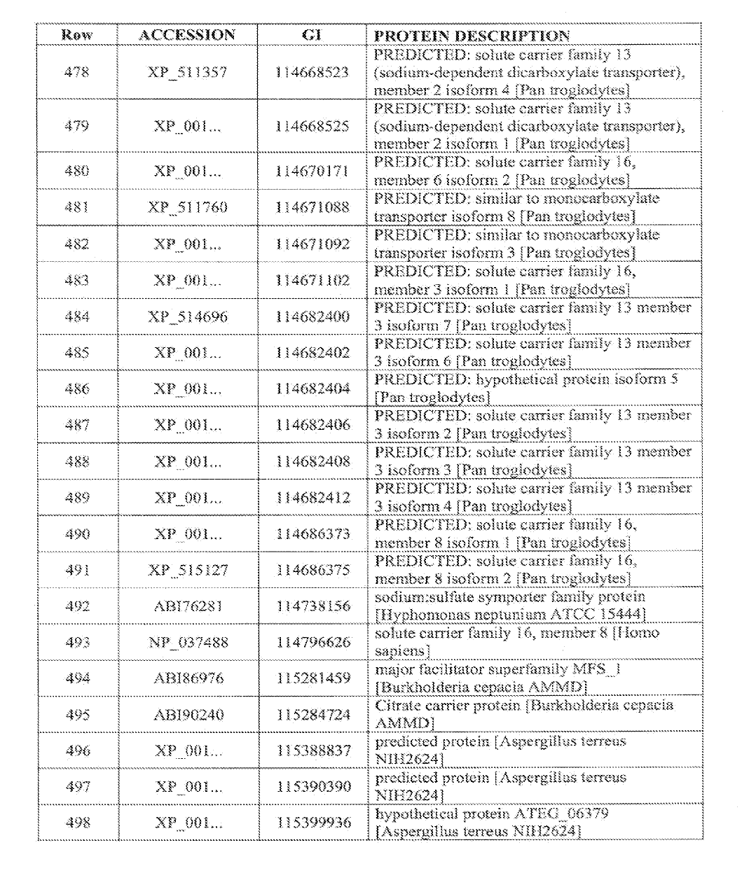

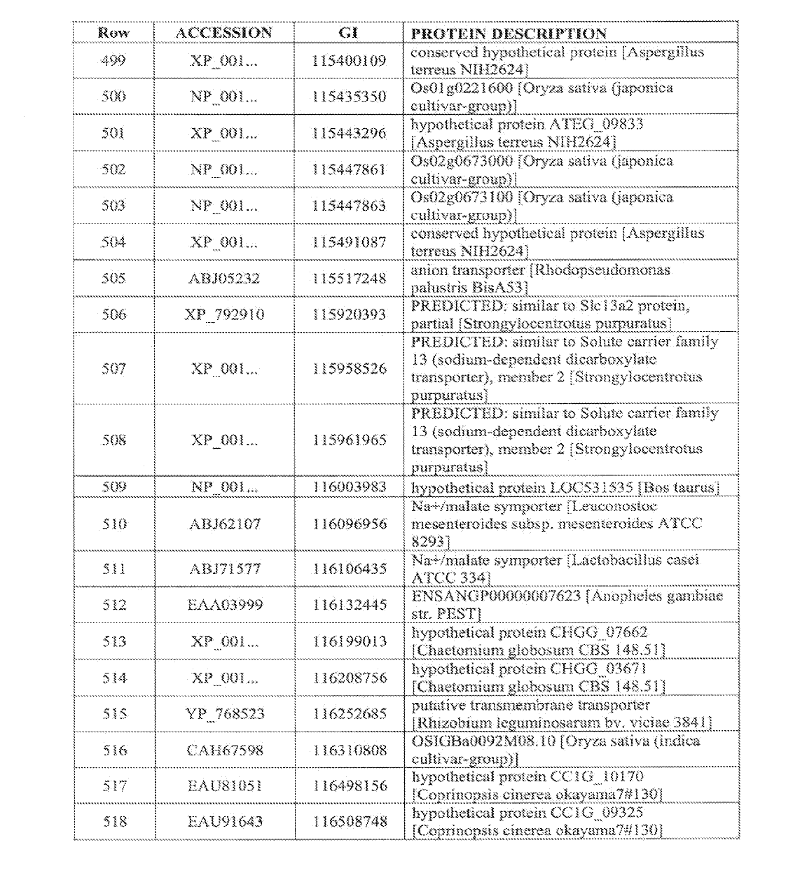

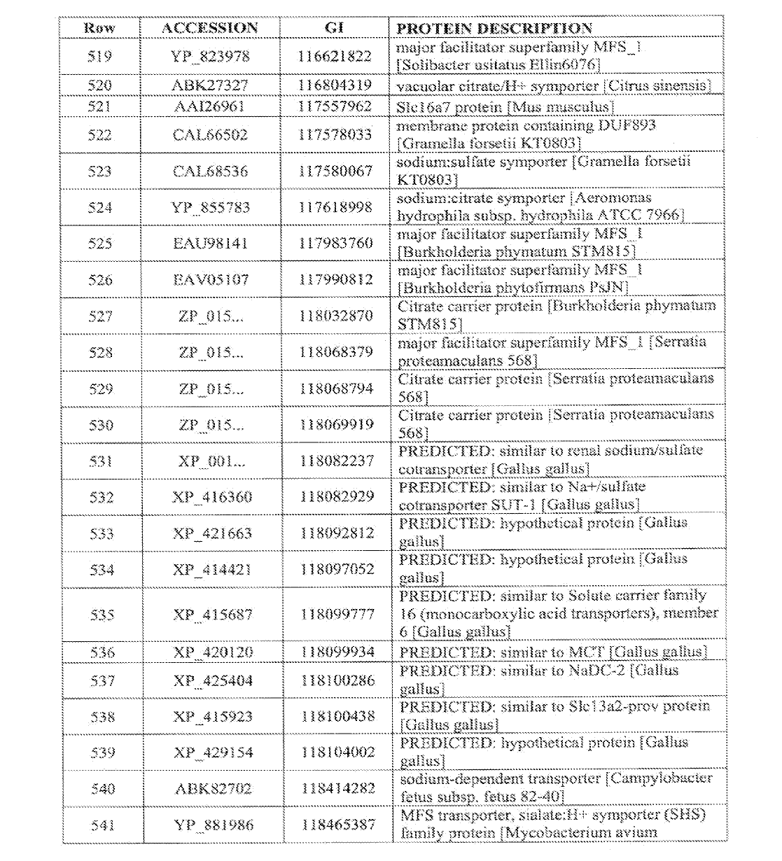

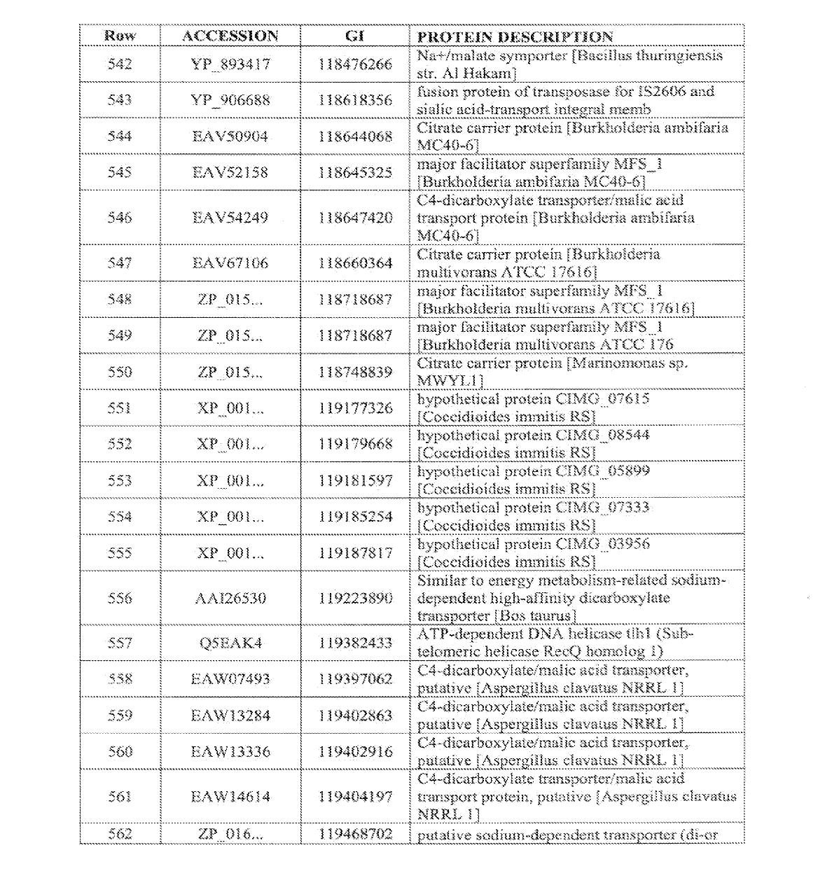

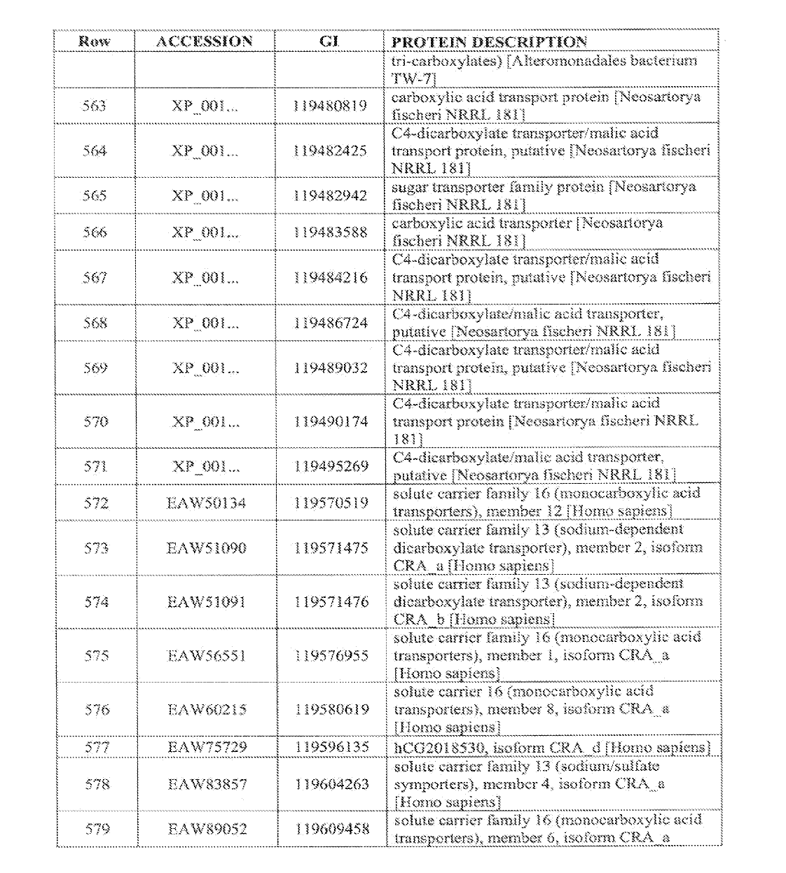

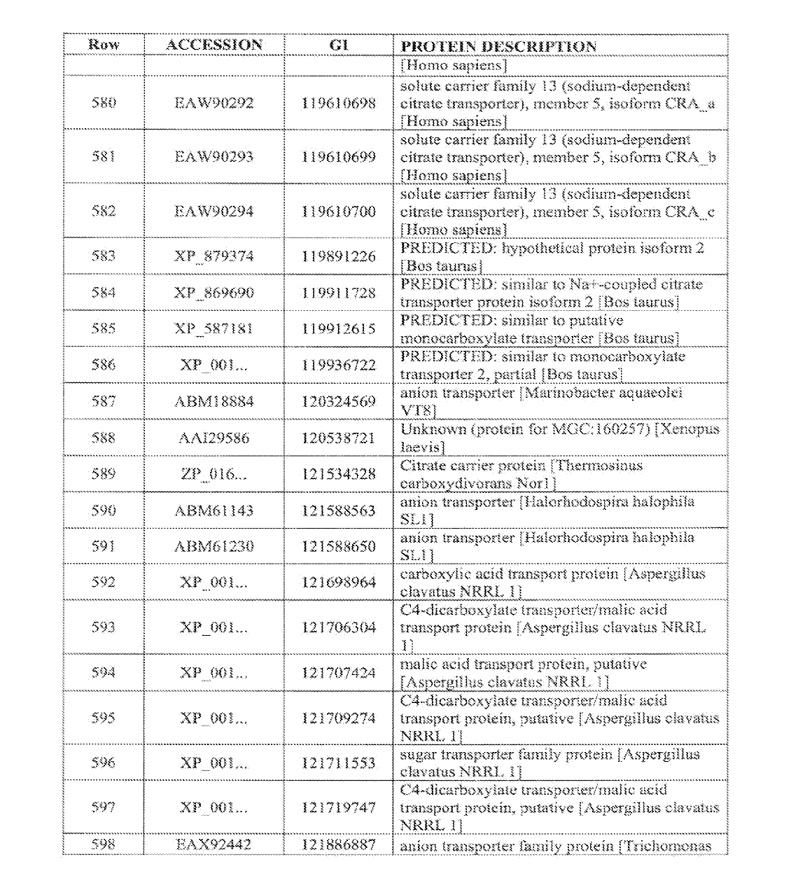

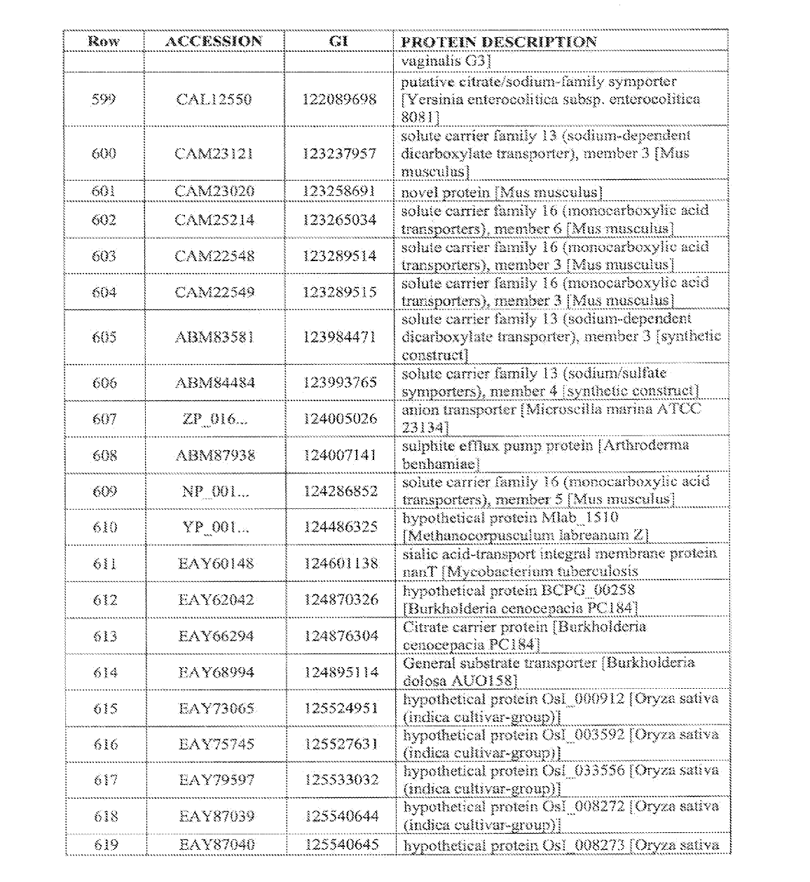

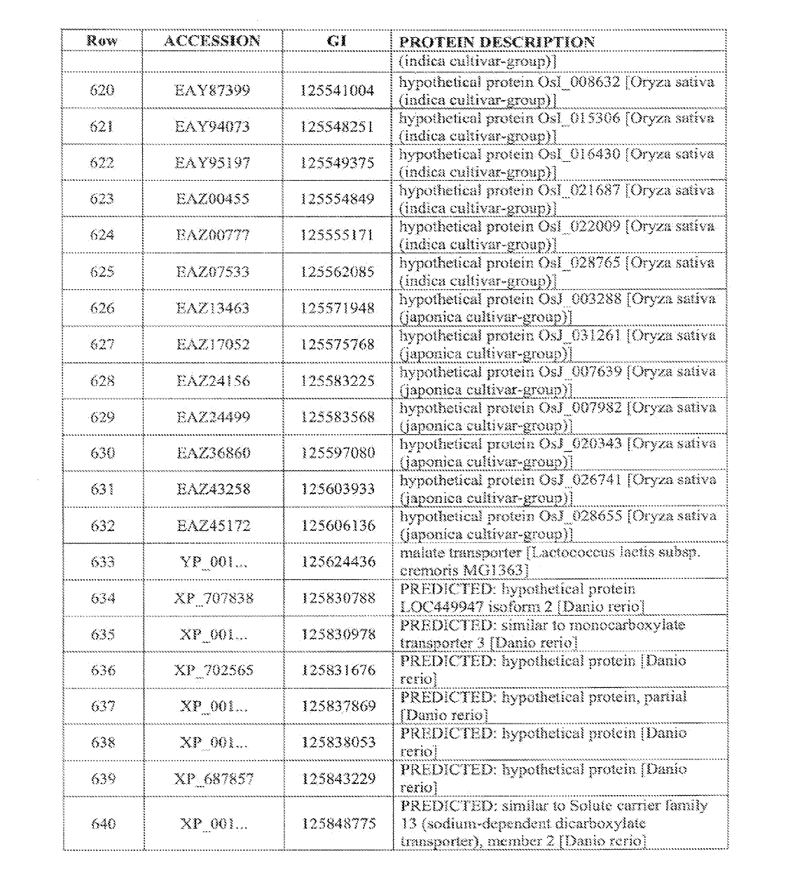

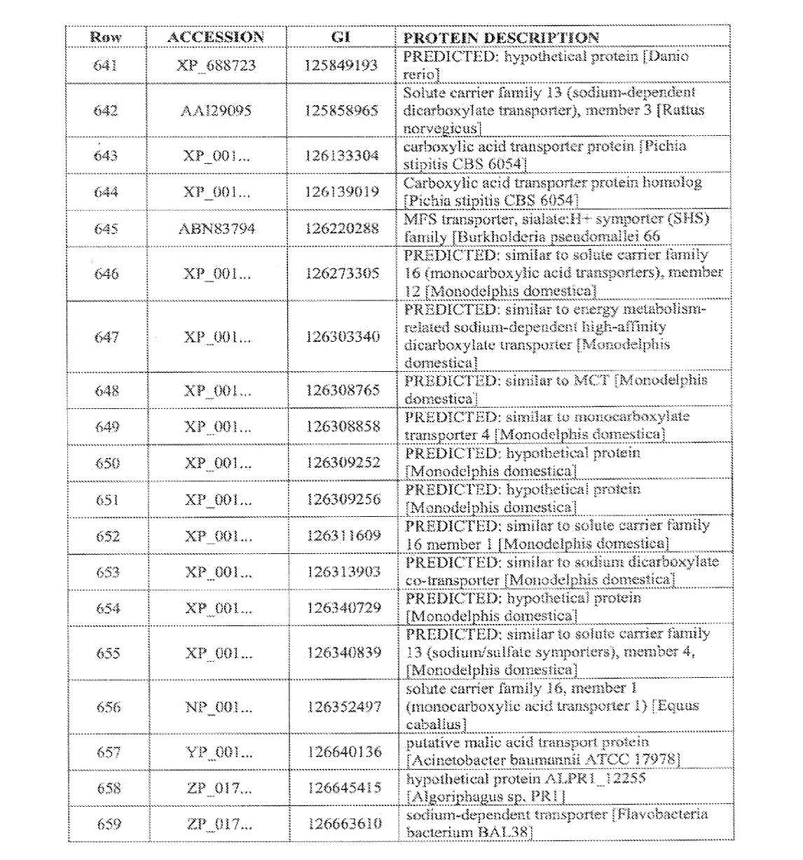

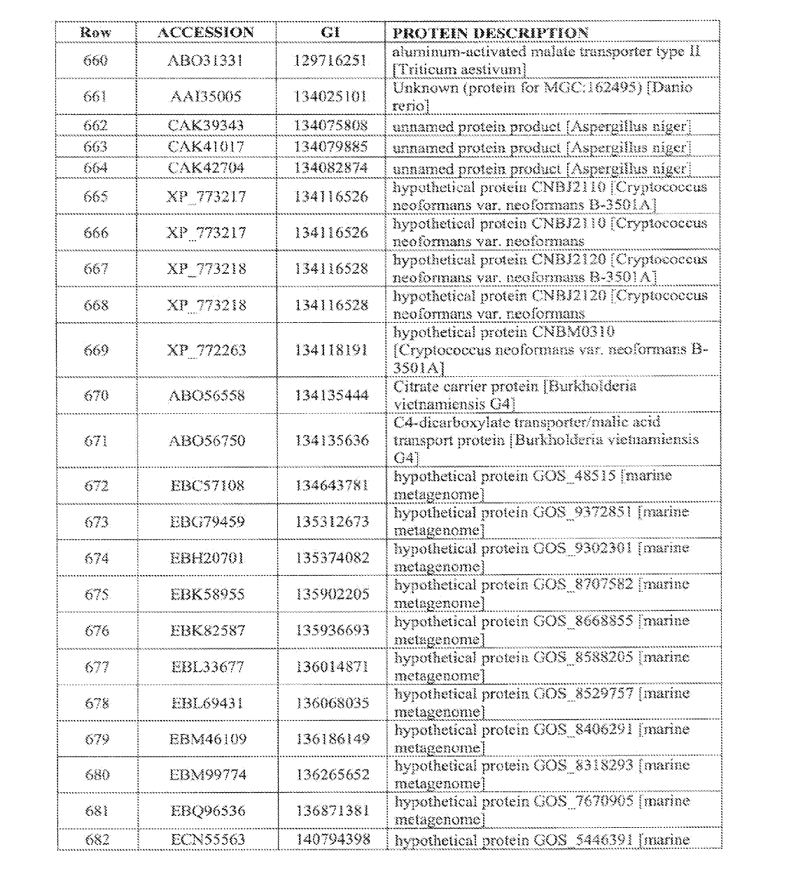

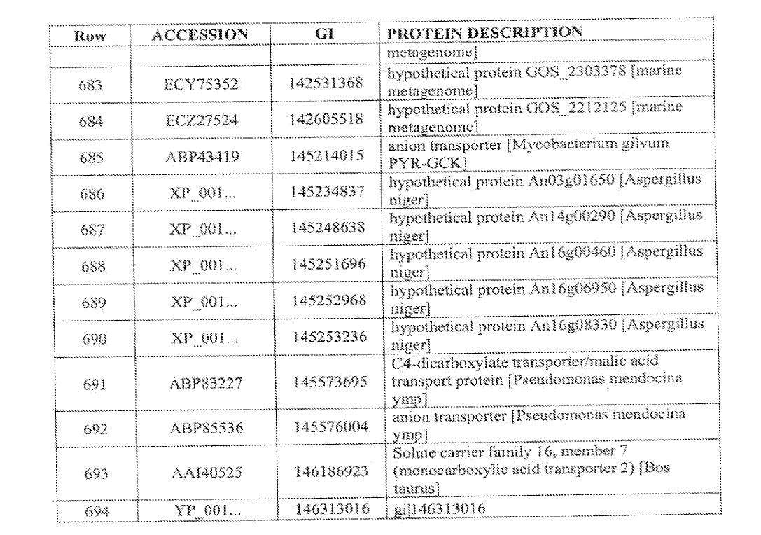

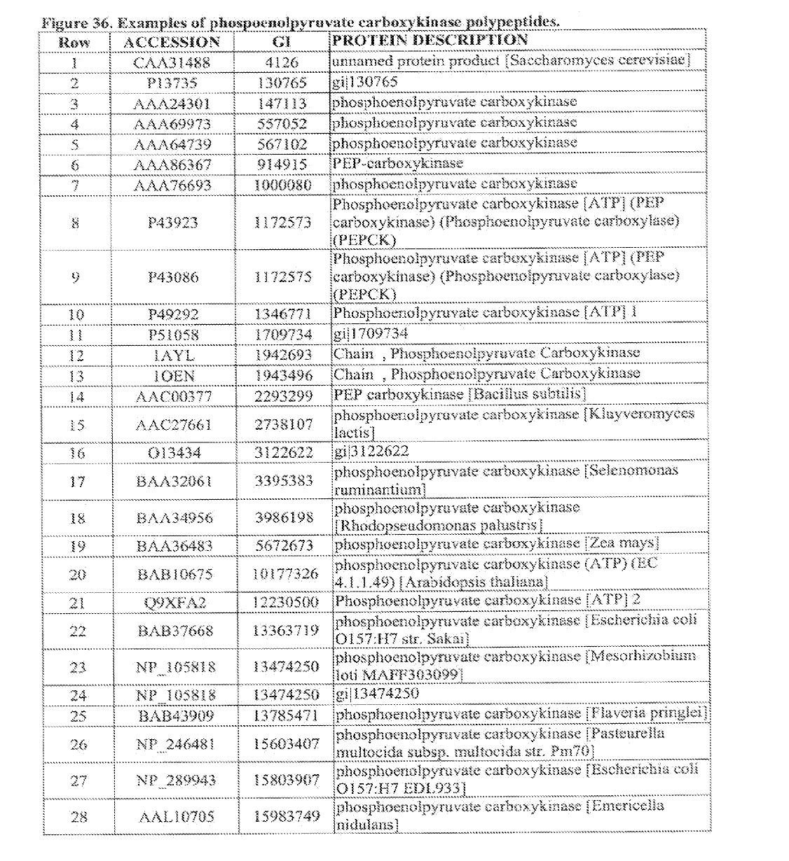

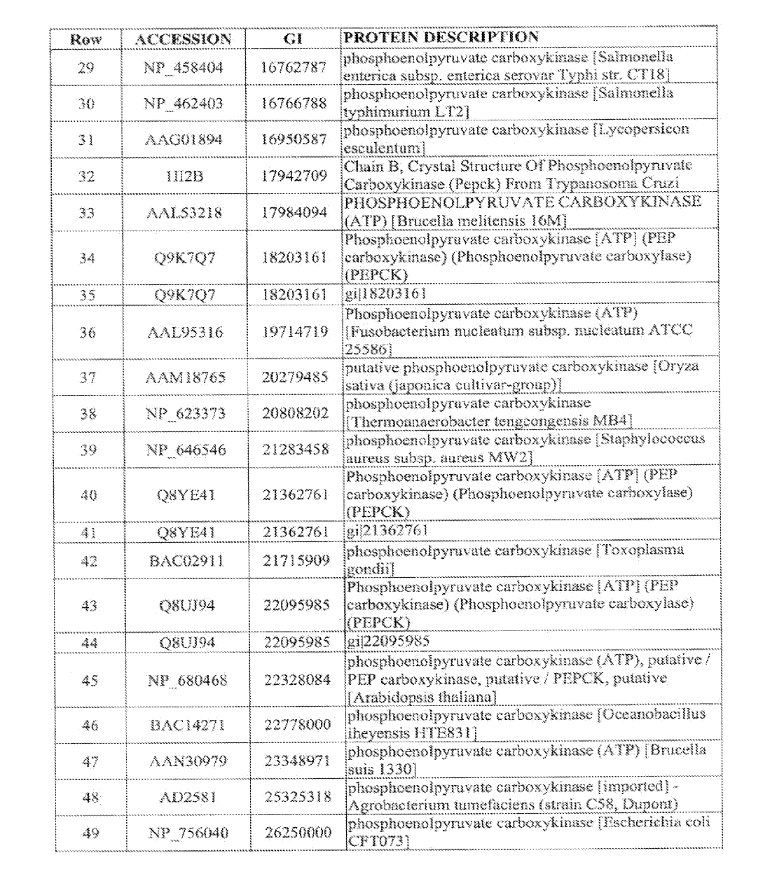

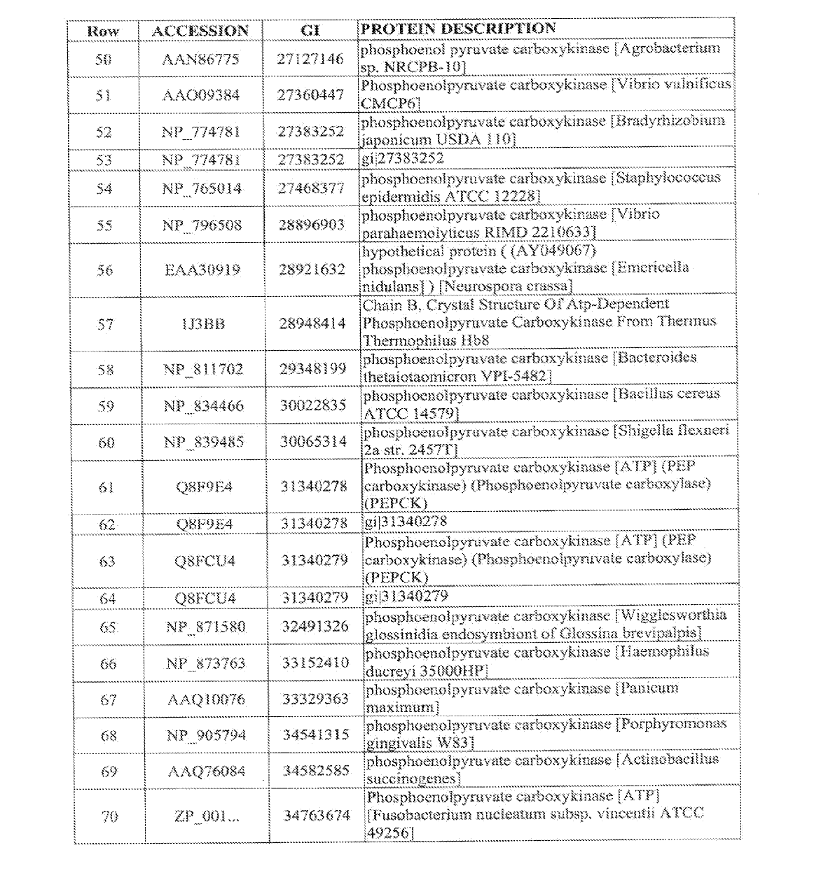

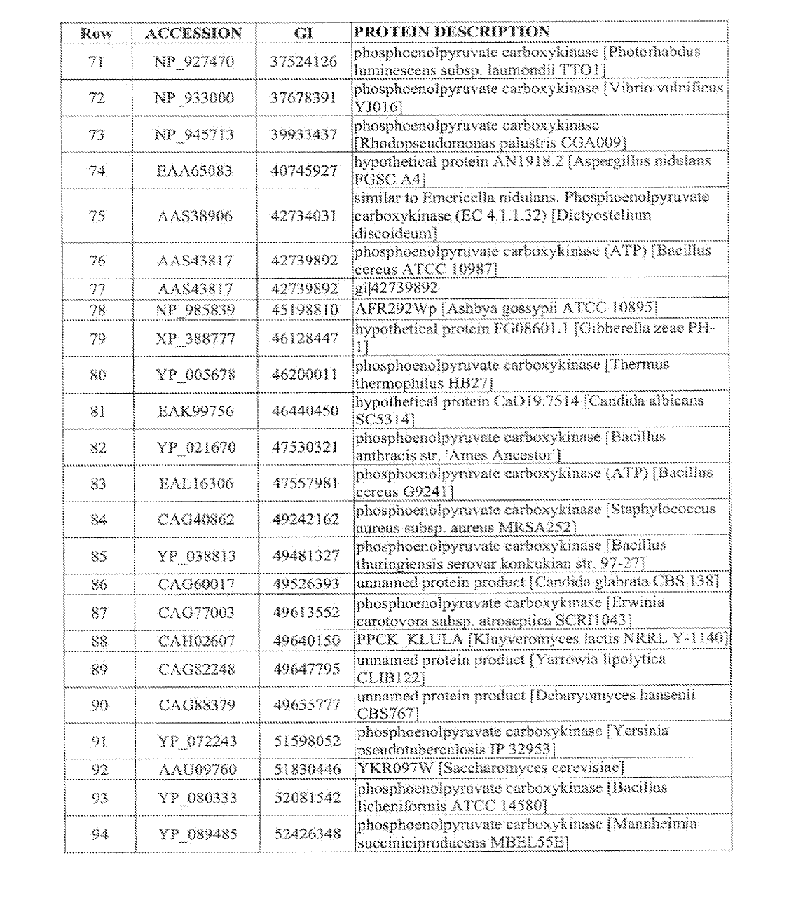

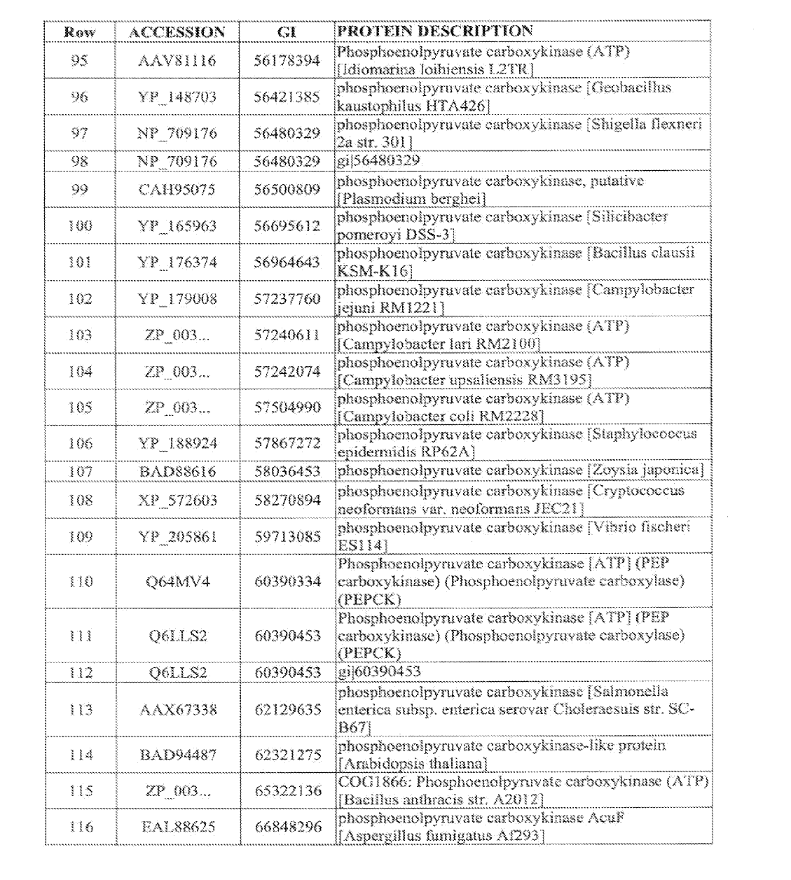

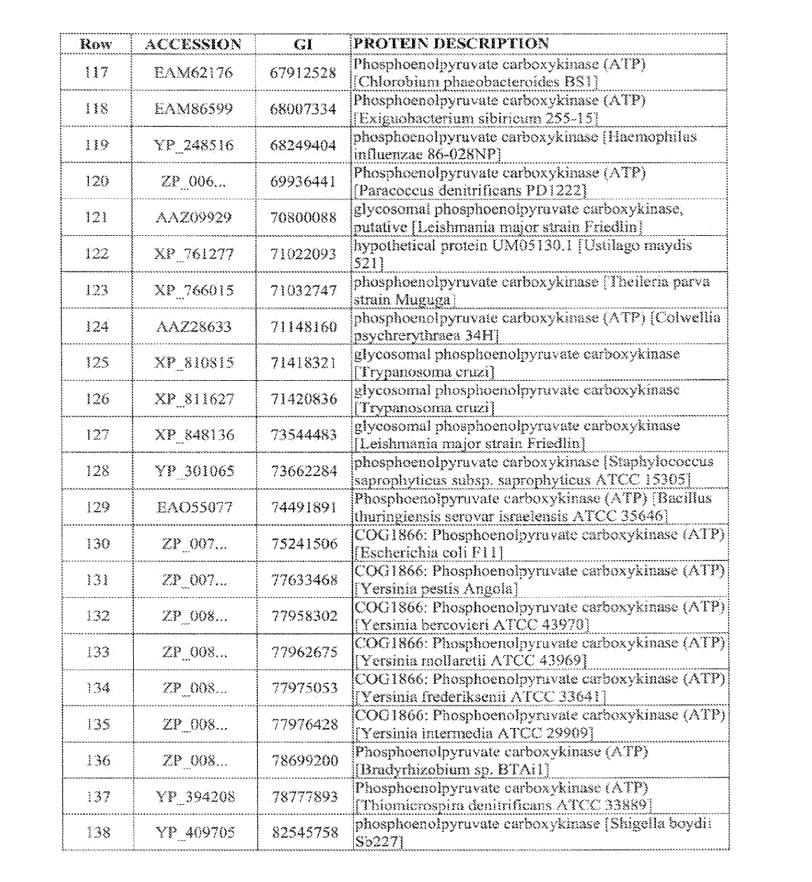

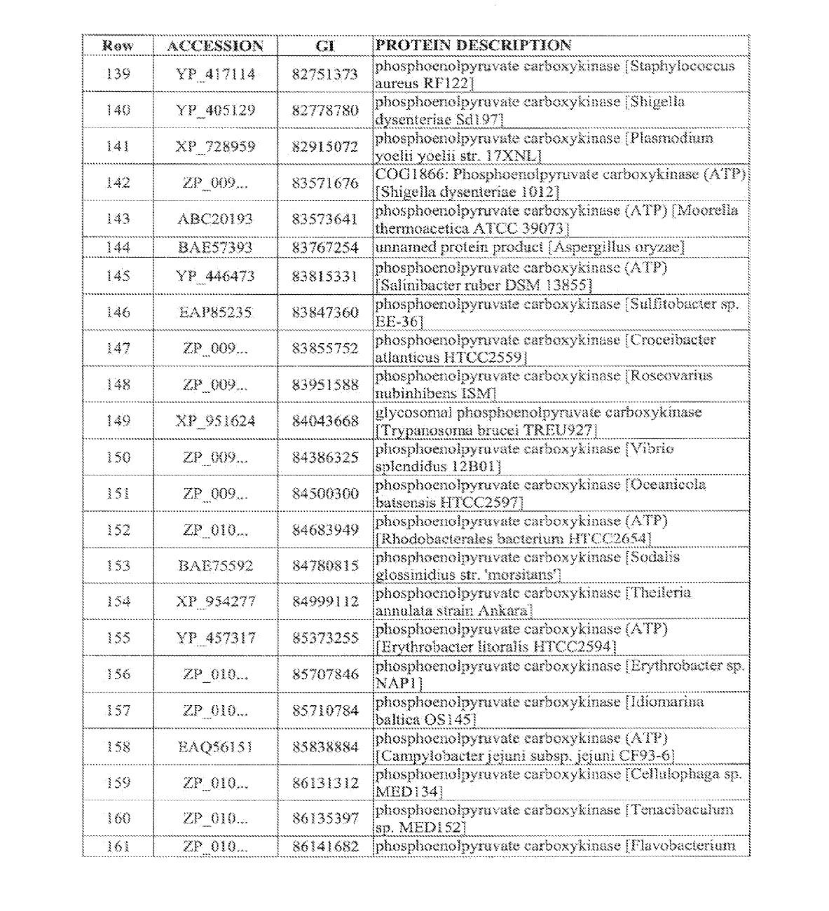

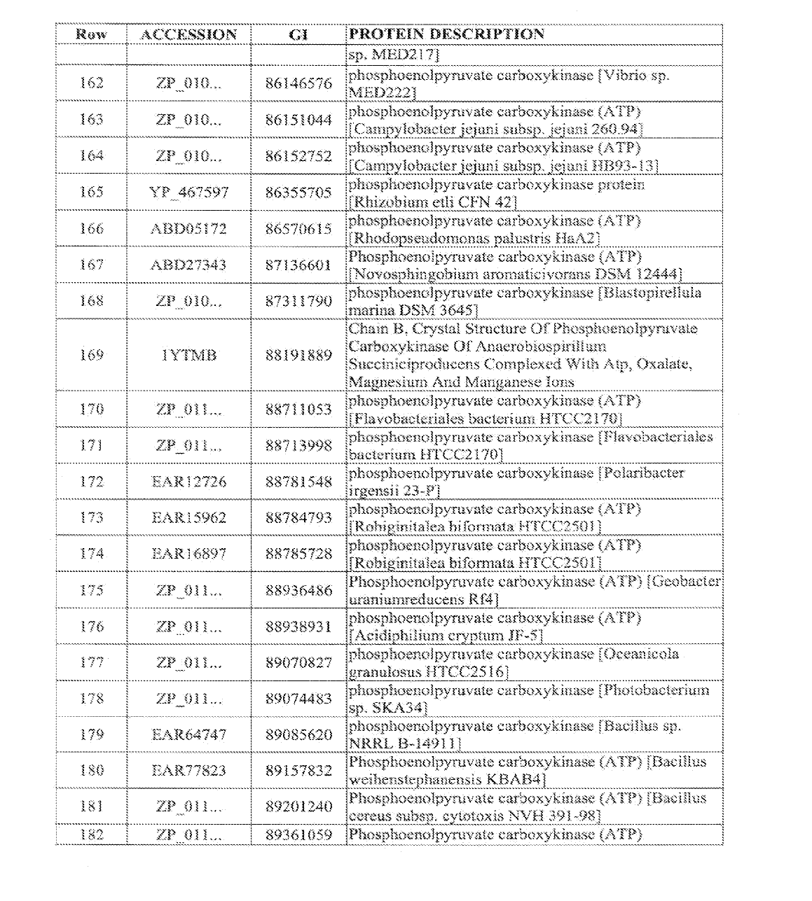

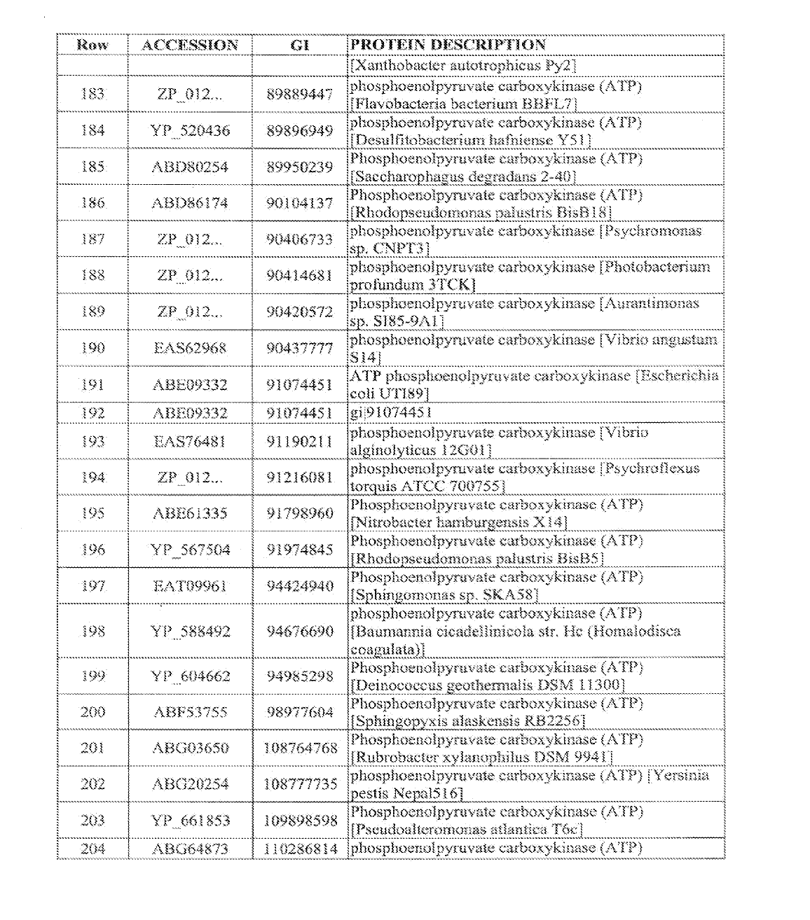

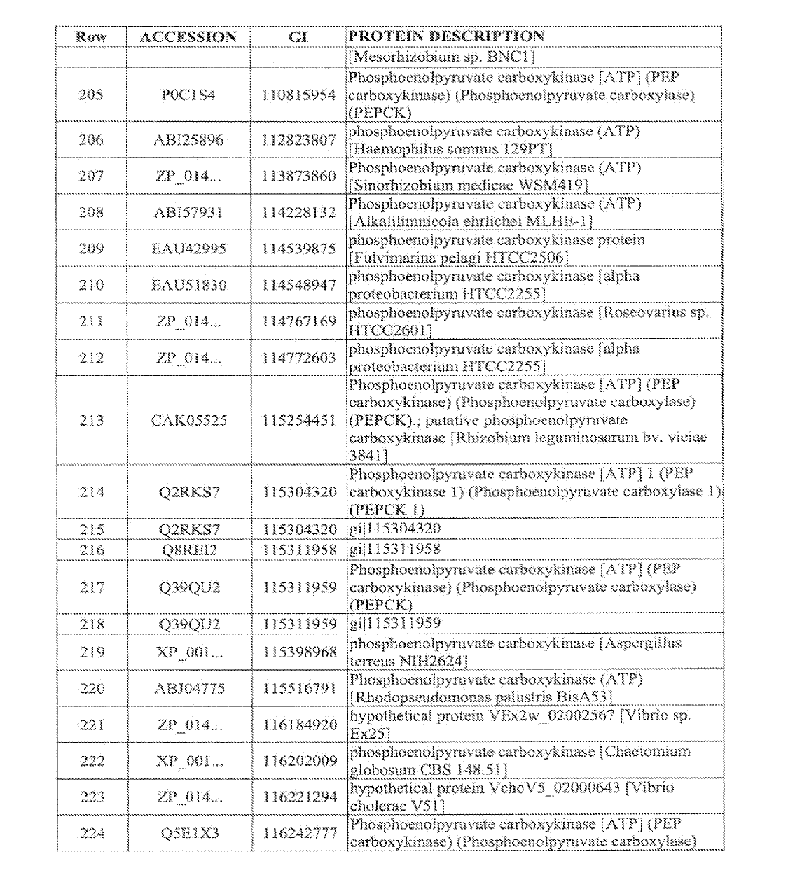

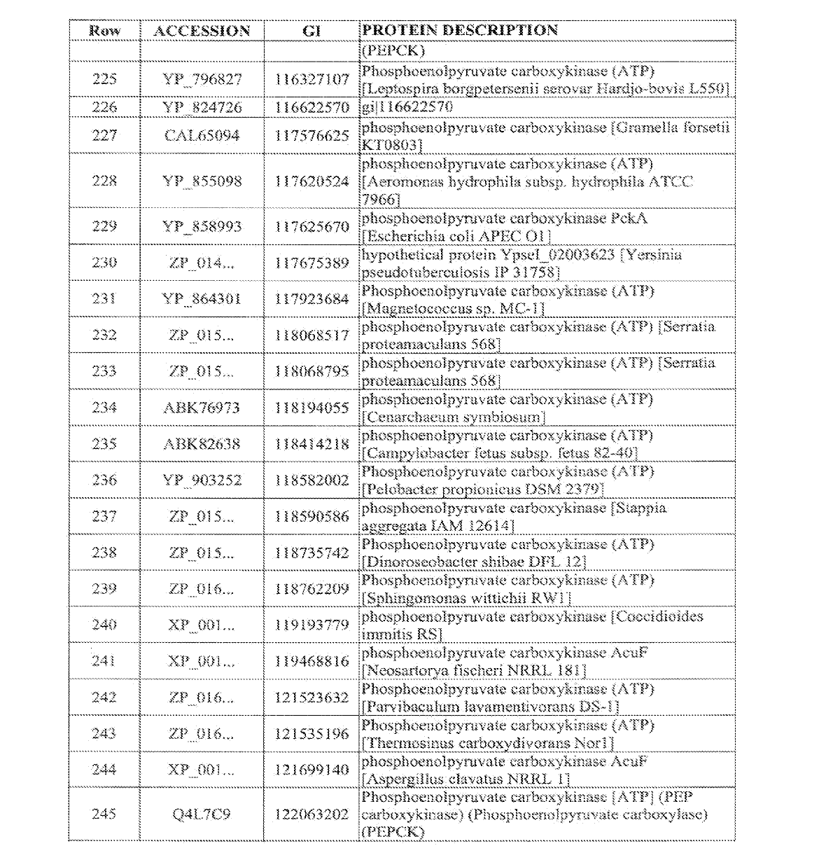

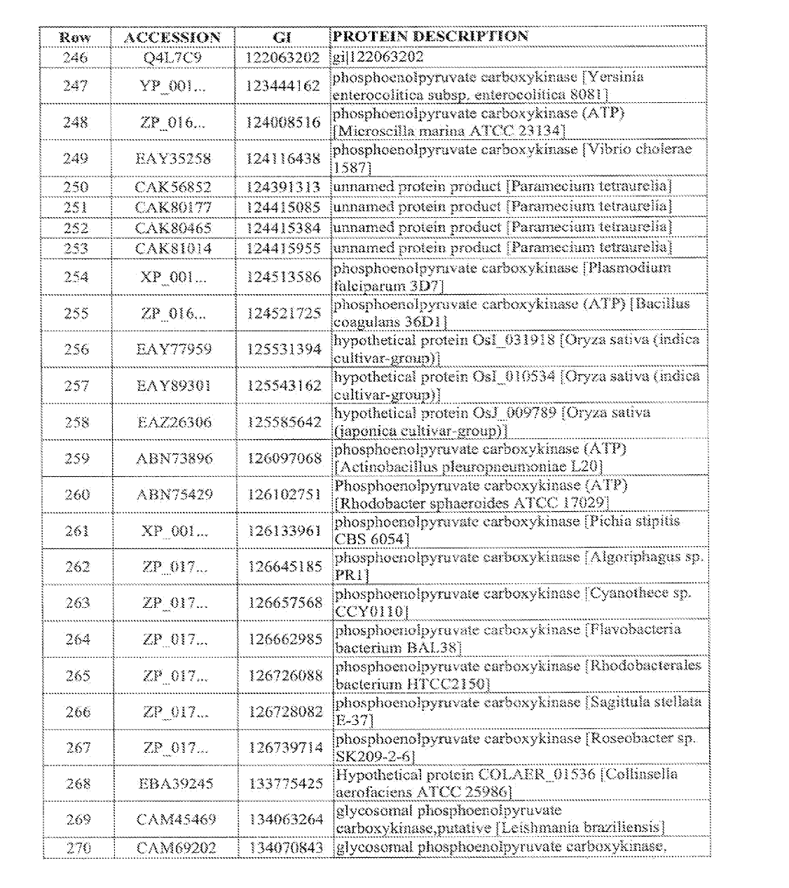

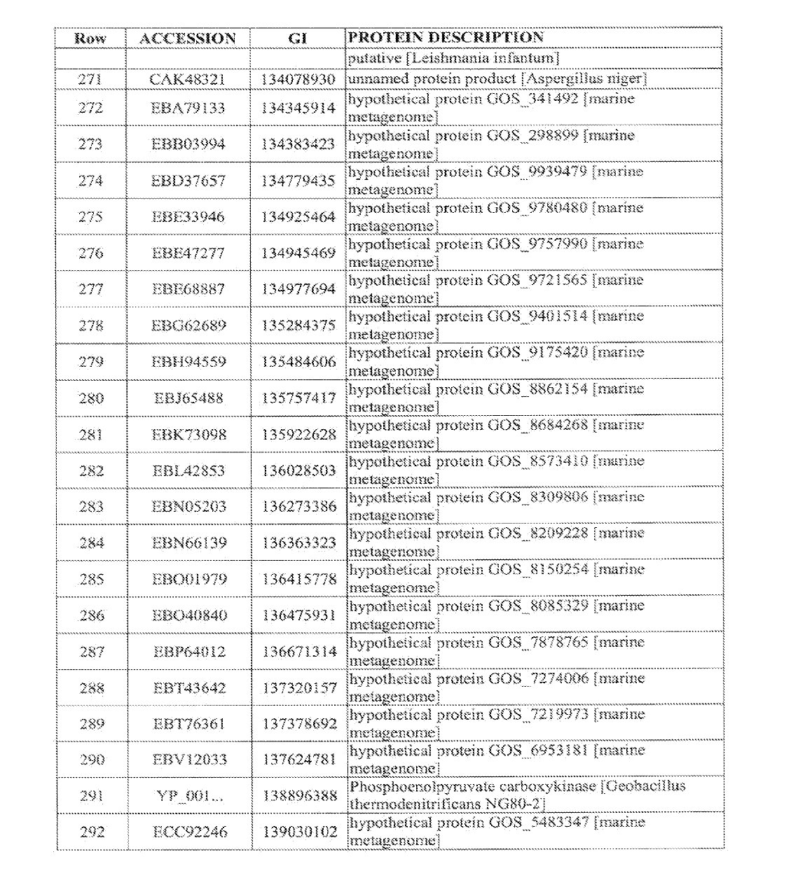

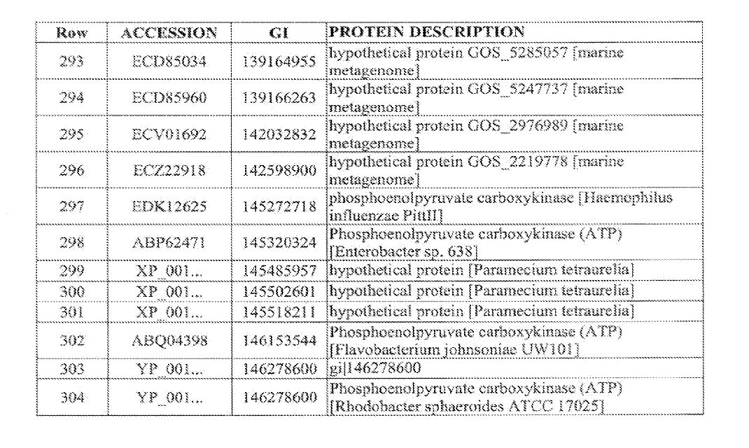

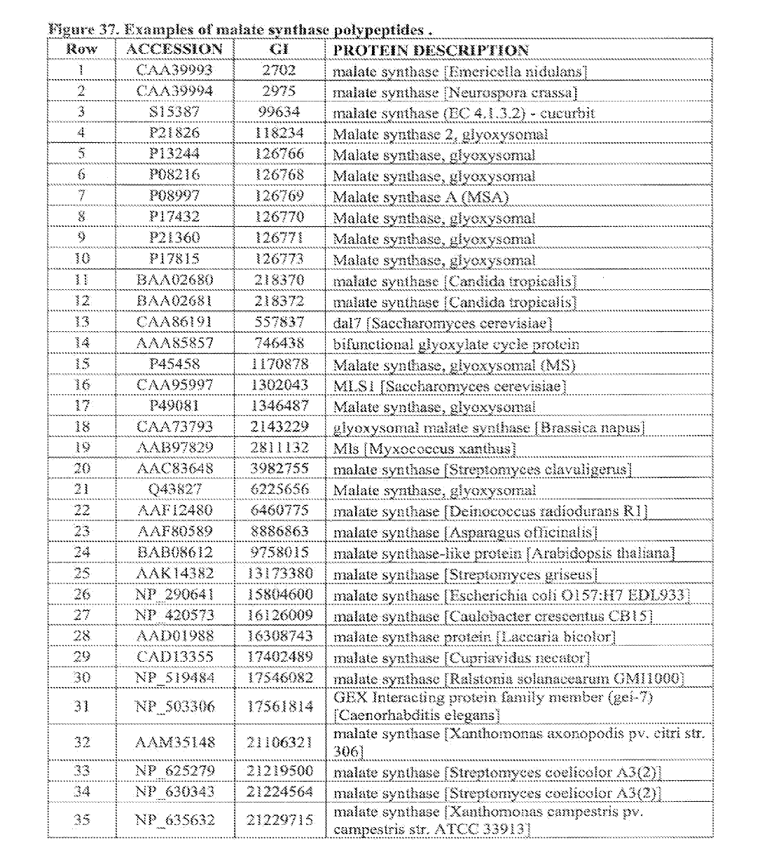

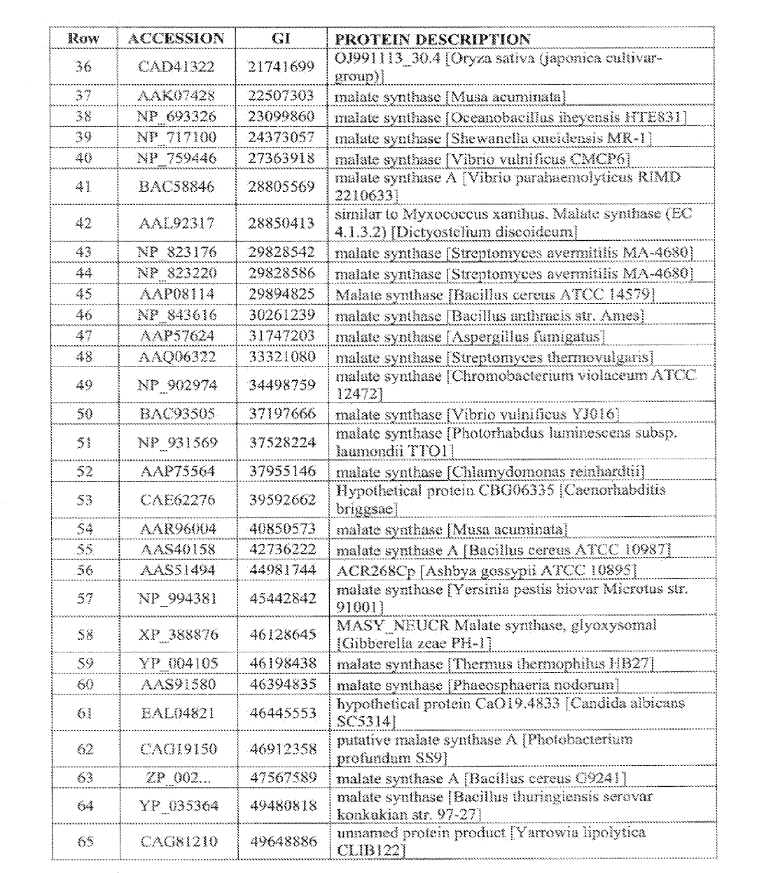

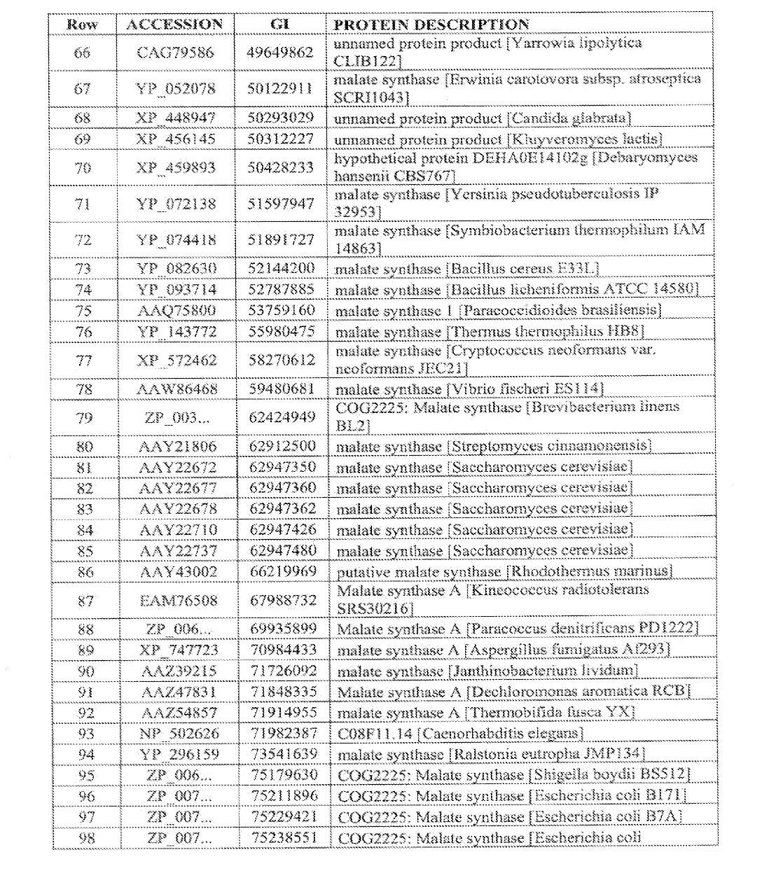

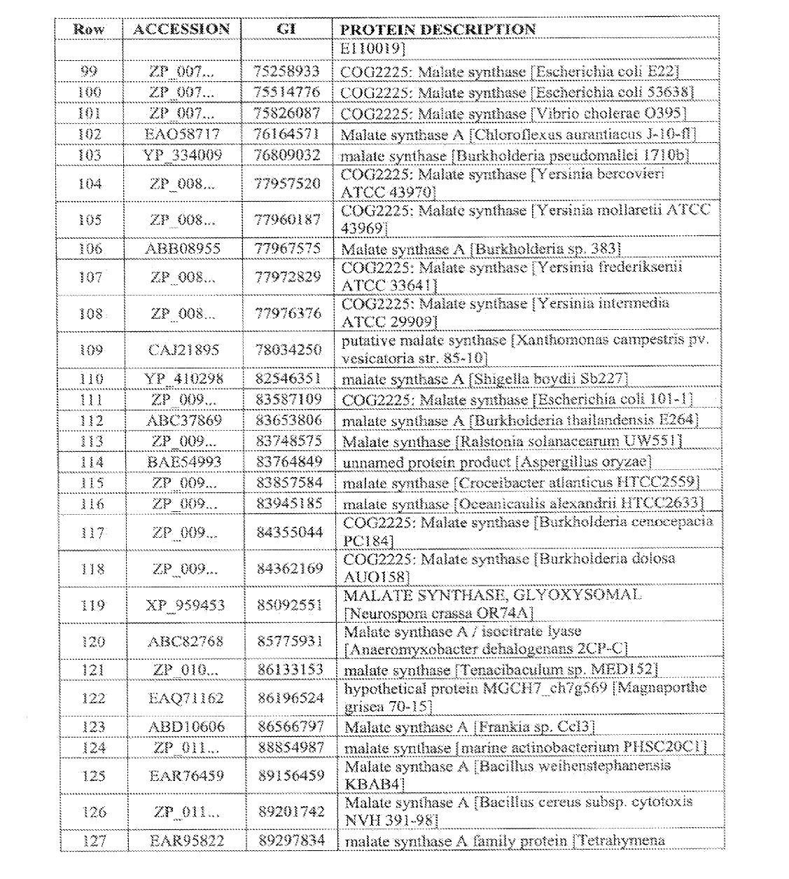

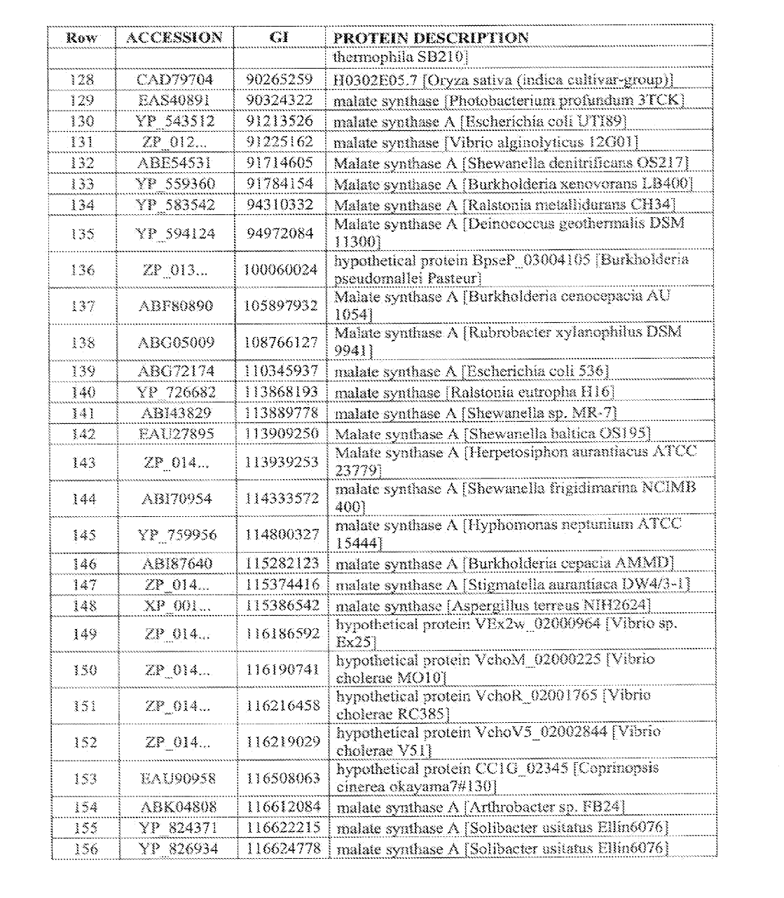

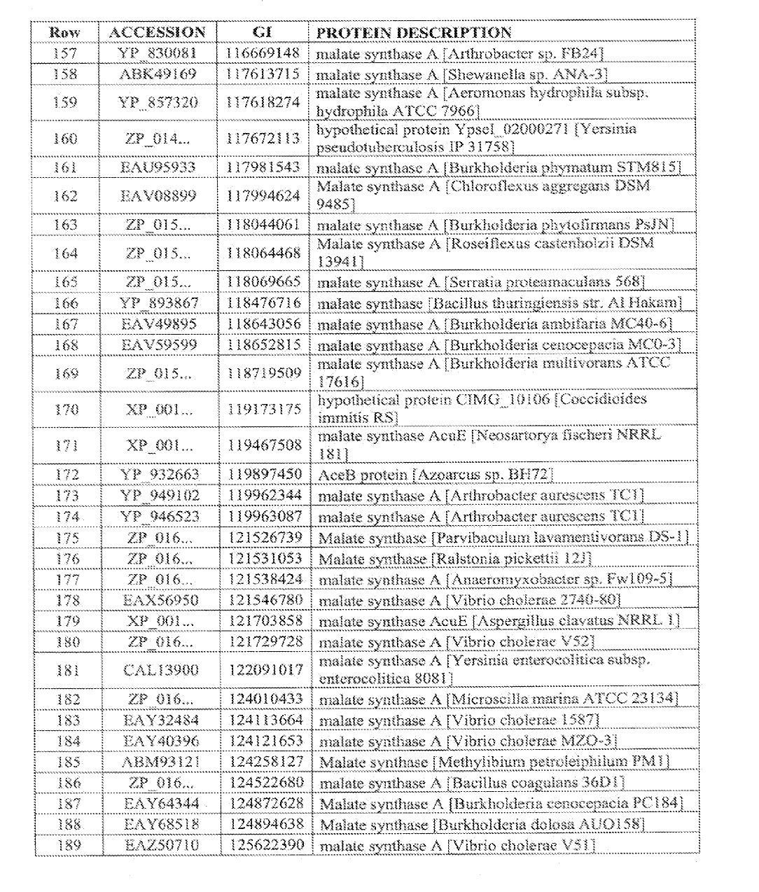

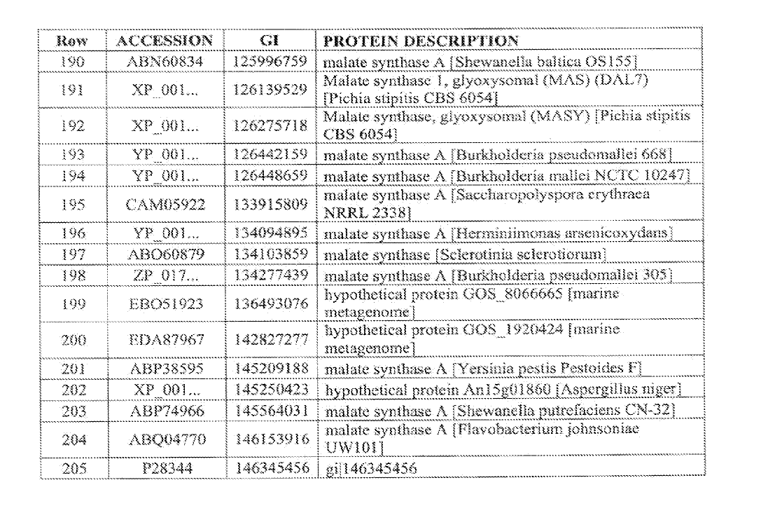

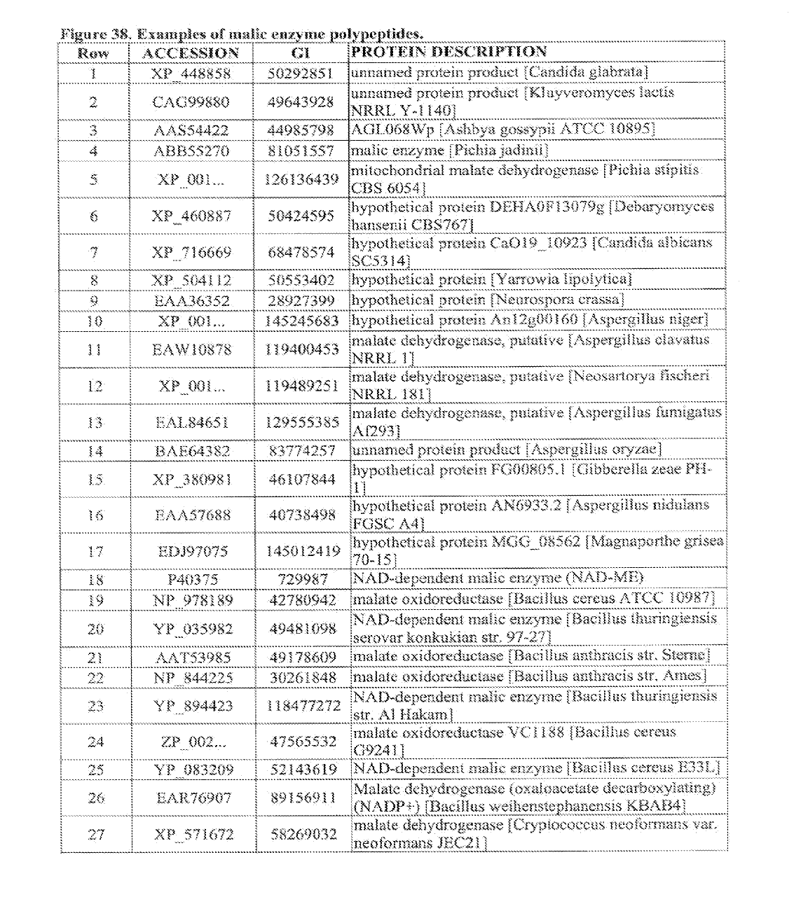

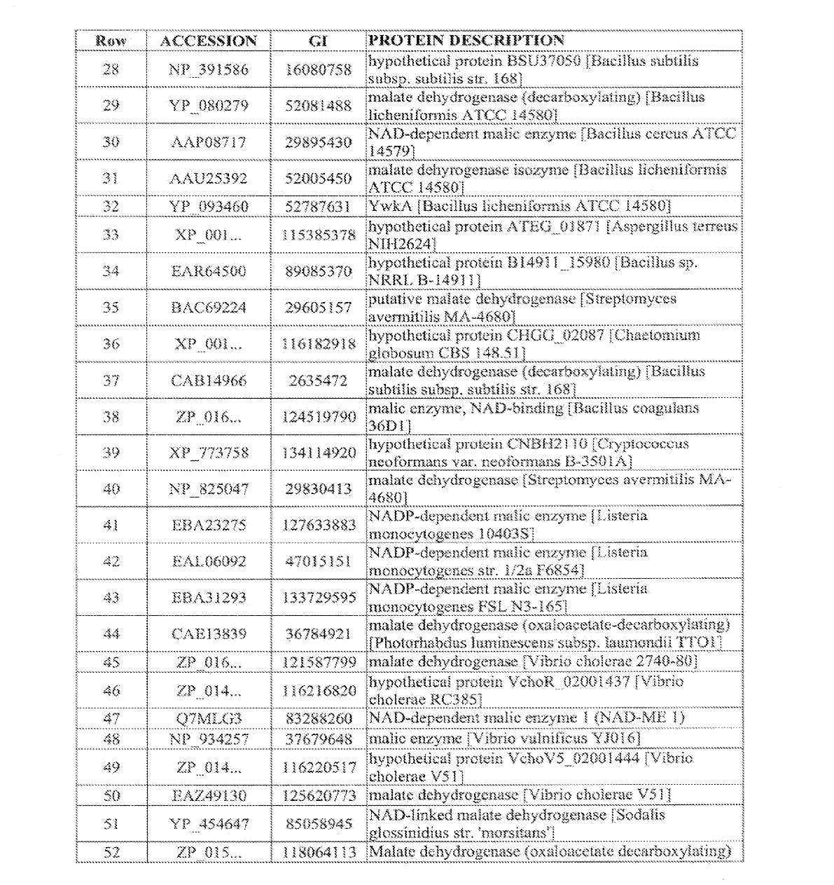

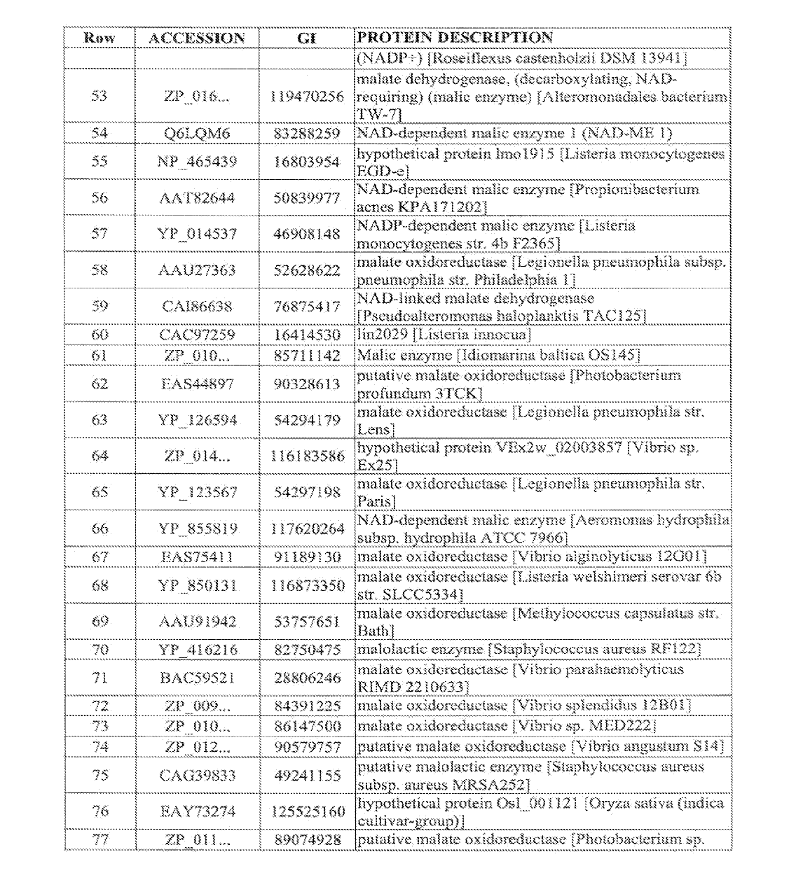

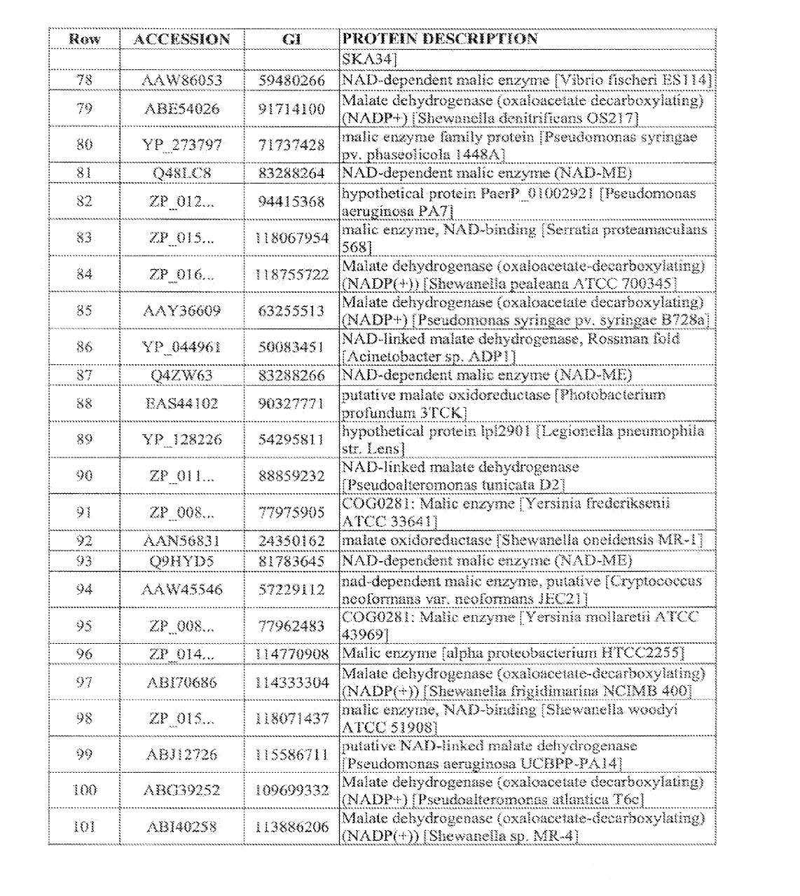

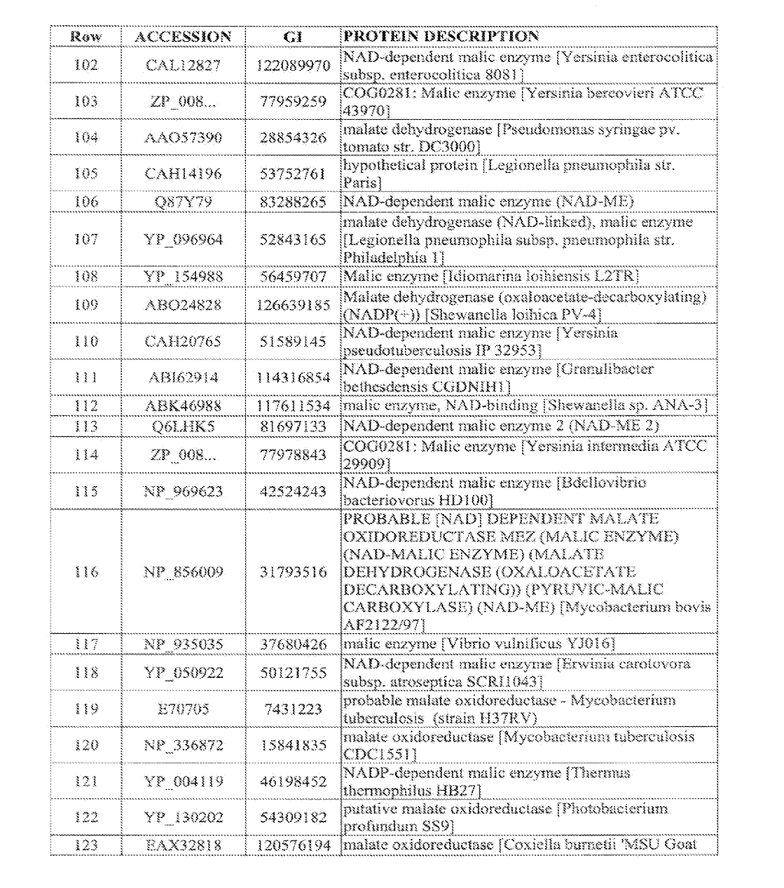

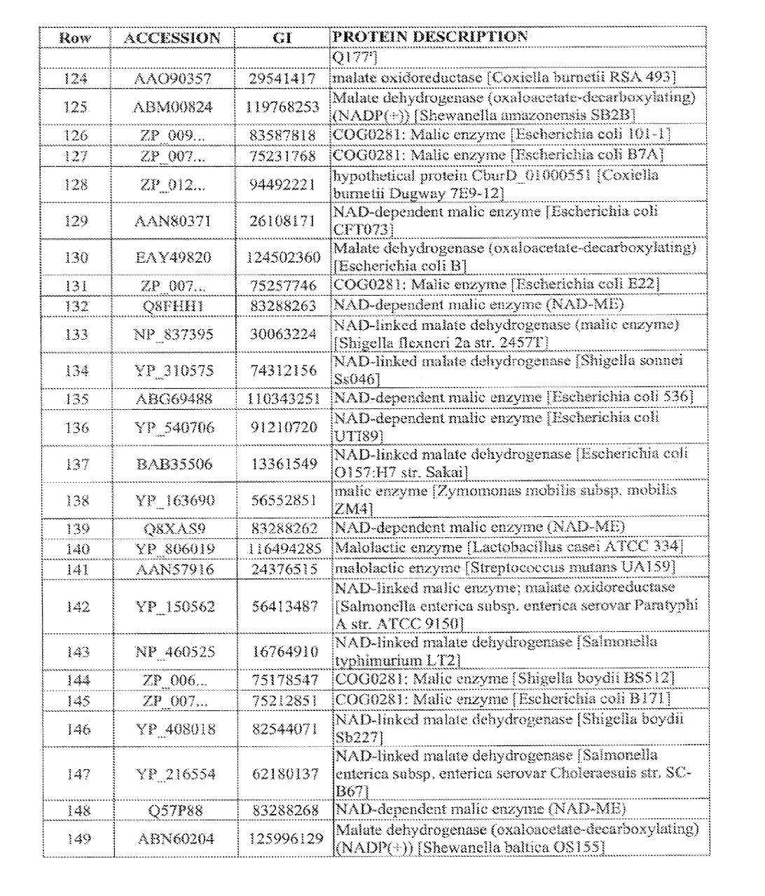

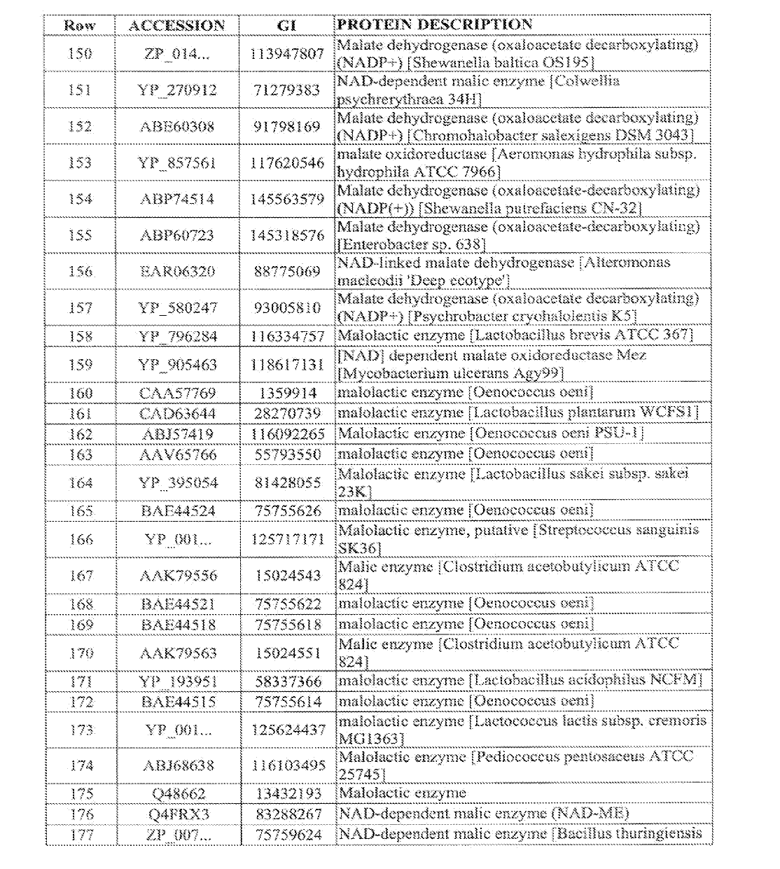

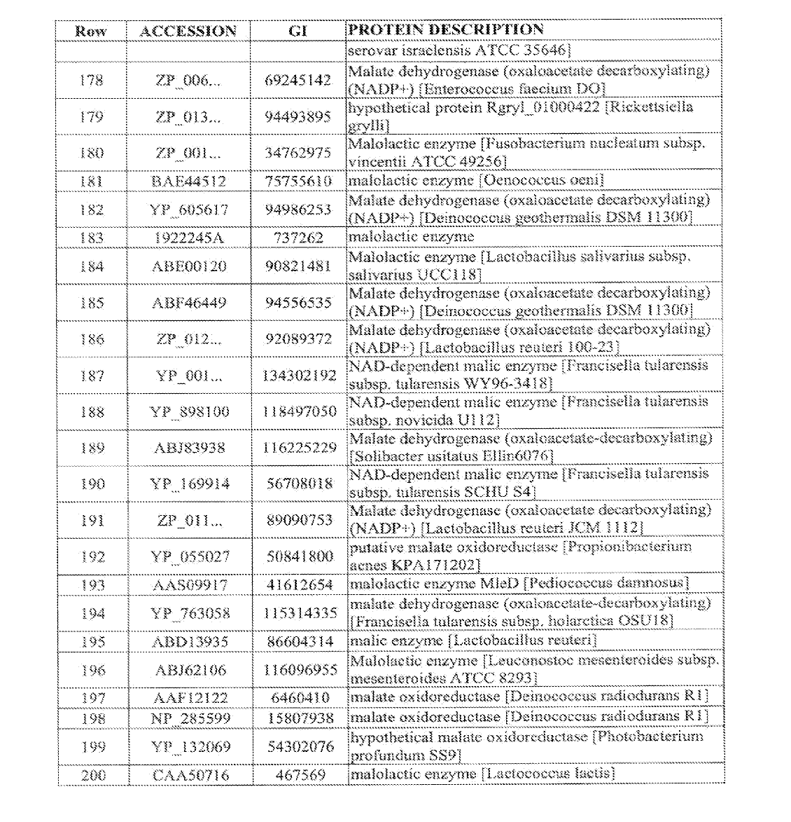

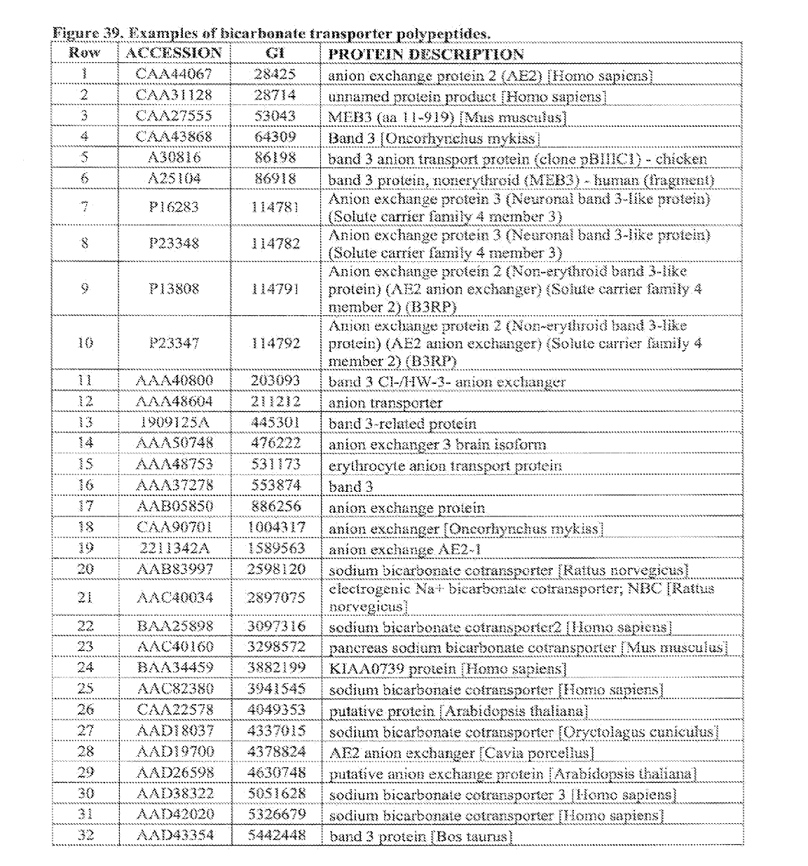

Anaplerotic polypeptides: “Anaplerotic polypeptides” provide activities that function in the carboxylation of the three carbon (C3) metabolic intermediates phosphoenolpyruvate and pyruvate to oxaloacetate, a C4 precursor of useful dicarboxylic acids such as malic acid, fumaric acid, succinic acid, and tartaric acid. In some cases, anaplerotic polypeptides are enzymes that catalyze particular steps in a synthesis pathway that ultimately produces oxaloacetate. In some embodiments, anaplerotic polypeptides may be polypeptides that do not themselves catalyze synthetic reactions, but that regulate expression and/or activity of other polypeptides that do so. For example, anaplerotic polypeptides include, among others, pyruvate carboxylase (PYC) polypeptides, phosphoenolpyruvate carboxylase (PPC) polypeptides, phosphoenolpyruvate carboxykinase (PCK) polypeptides, pyruvate kinase (PYK) polypeptides, biotin protein ligase (BPL) polypeptides, biotin transport protein (VHT) polypeptides, bicarbonate transport activity, and carbonic anhydrase polypeptides. Synthetic anaplerotic polypeptides include PYC, PPC, PCK, and PYK polypeptides. A modification that increases the activity of an anaplerotic polypeptide is one which increases the enzymatic, transport or other functional activity of the polypeptide or one which increases the amount of the polypeptide present in a cell or a cell compartment. Polypeptides that do not catalyze a biosynthetic reaction yet function in the carboxylation of the C3 metabolic intermediates phosphoenolpyruvate and pyruvate to oxaloacetate include: BPL, VHT, bicarbonate transport, and carbonic anhydrase polypeptides. Thus, a modification that increases or decreases the activity of one of these polypeptides may also modify the level of carboxylation of C3 metabolic intermediates. Example anaplerotic polypeptides are represented by the pyruvate carboxylase (PYC) polypeptides, phosphoenolpyruvate carboxylase (PPC) polypeptides, phosphoenolpyruvate carboxykinase (PCK) polypeptides, pyruvate kinase (PYK) polypeptides, biotin protein ligase (BPL) polypeptides, biotin transport protein (VHT) polypeptides, bicarbonate transporter polypeptides, the carbonic anhydrase polypeptides in FIG. 29; polypeptides that have at least 99%, 98%, 97%, 96%, 95%, 94%, 93%, 92%, 91%, 90%, 85%, 80%, 75% identity to pyruvate carboxylase (PYC) polypeptides, phosphoenolpyruvate carboxylase (PPC) polypeptides, phosphoenolpyruvate carboxykinase (PCK) polypeptides, pyruvate kinase (PYK) polypeptides, biotin protein ligase (BPL) polypeptides, biotin transport protein (VHT) polypeptides, bicarbonate transporter polypeptides, the carbonic anhydrase polypeptides represented in FIG. 29; polypeptides represented by the Genbank GI numbers in FIGS. 32, 33, 36, 39, 40, 45, 46, and 48; and polypeptides that have at least 99%, 98%, 97%, 96%, 95%, 94%, 93%, 92%, 91%, 90%, 85%, 80%, 75% identity to a polypeptide represented by the Genbank GI numbers in FIGS. 32, 33, 36, 39, 40, 45, 46, and 48.

ATP-citrate lyase polypeptides: “ATP-citrate lyase polypeptides” catalyze the cytosolic, reversible reaction:

citrate+CoA+ATP→acetyl−CoA+oxaloacetate+ADP+Pᵢ. (EC 2.3.3.8)

The resulting acetyl-CoA often serves as a substrate for fatty acid synthesis or the malate synthase reaction of the glyoxylate cycle. Examples of ATP-citrate lyase polypeptides subunits 1 and 2 are represented by the Genbank GI numbers in FIGS. 41a and 41b and the ATP-citrate lyase polypeptides in FIG. 29. In some cases, an ATP-citrate lyase polypeptide has an amino acid sequence that is at least about 75%, 85%, 90%, 95% or 100% identical to that of a polypeptide identified by the Genbank GI numbers in FIG. 41a or 41b or the ATP-citrate lyase polypeptides in FIG. 29.

Bicarbonate transport (BCT) polypeptides: “Bicarbonate transport (BCT) polypeptides” facilitate the (reversible) movement of membrane impermeant HCO₃⁻ across biological membranes. Classes of BCT polypeptides include, but are not limited to, Cl⁻/HCO₃⁻ exchange, Na⁺/HCO₃⁻ co-transport, and Na⁺-dependent Cl⁻/HCO₃⁻ exchange polypeptides. BCT polypeptides are critical for the physiological processes of HCO₃⁻ metabolism and excretion, the regulation of pH, and the regulation of cell volume. Examples of bicarbonate transport (BCT) polypeptides are represented by the Genbank GI numbers in FIG. 39 and the BCT polypeptides in FIG. 29. In some cases, a bicarbonate transport (BCT) polypeptide has an amino acid sequence that is at least about 75%, 85%, 90%, 95% or 100% identical to that of a polypeptide represented by the Genbank GI numbers in FIG. 39 or a BCT polypeptide in FIG. 29.

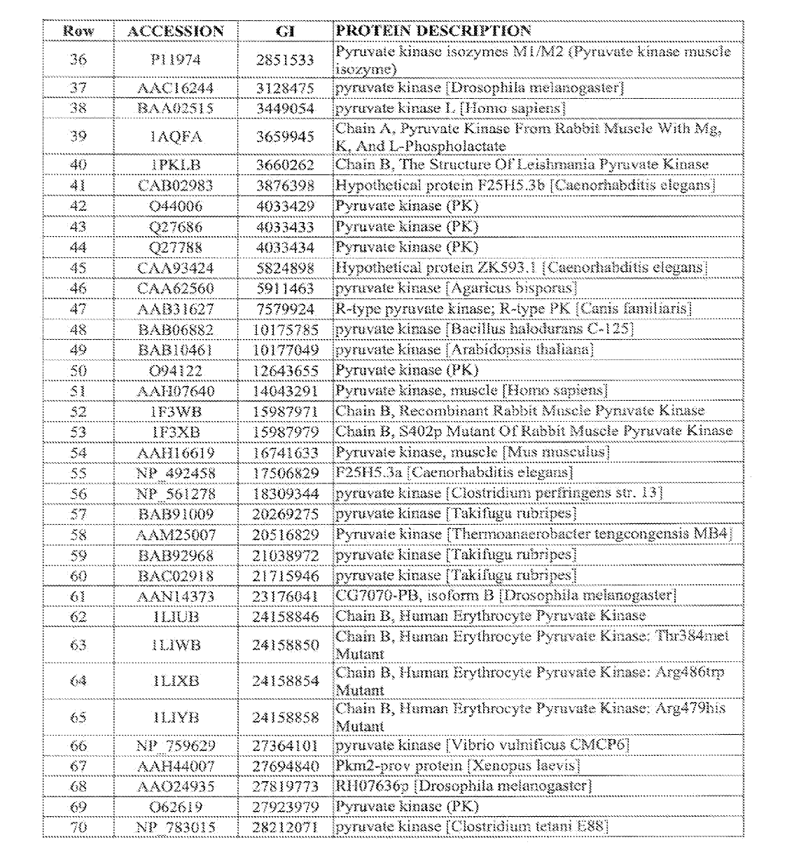

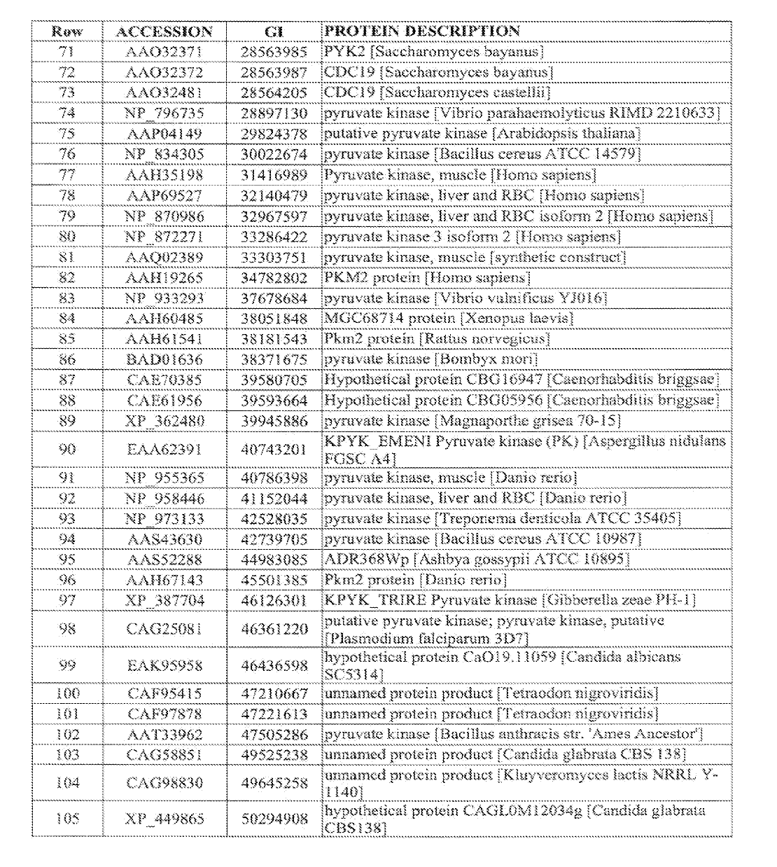

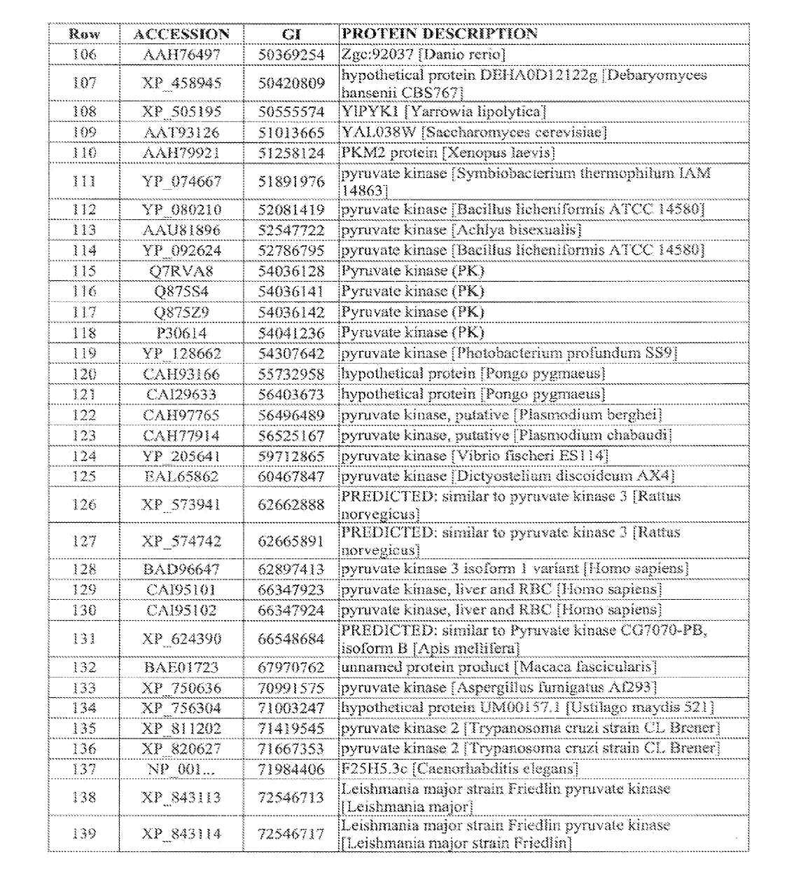

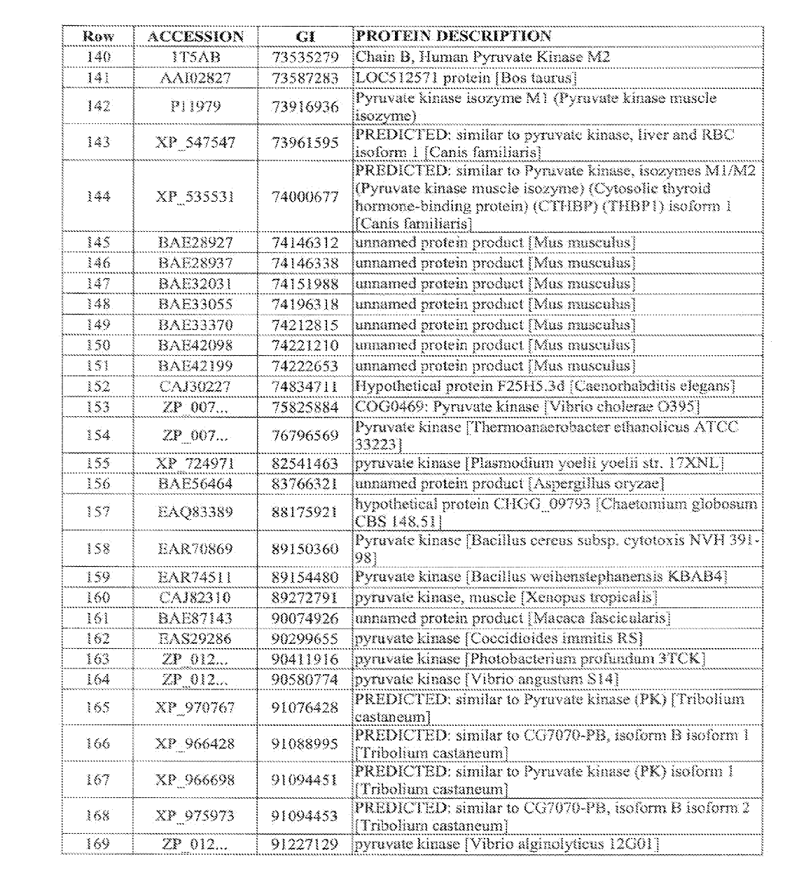

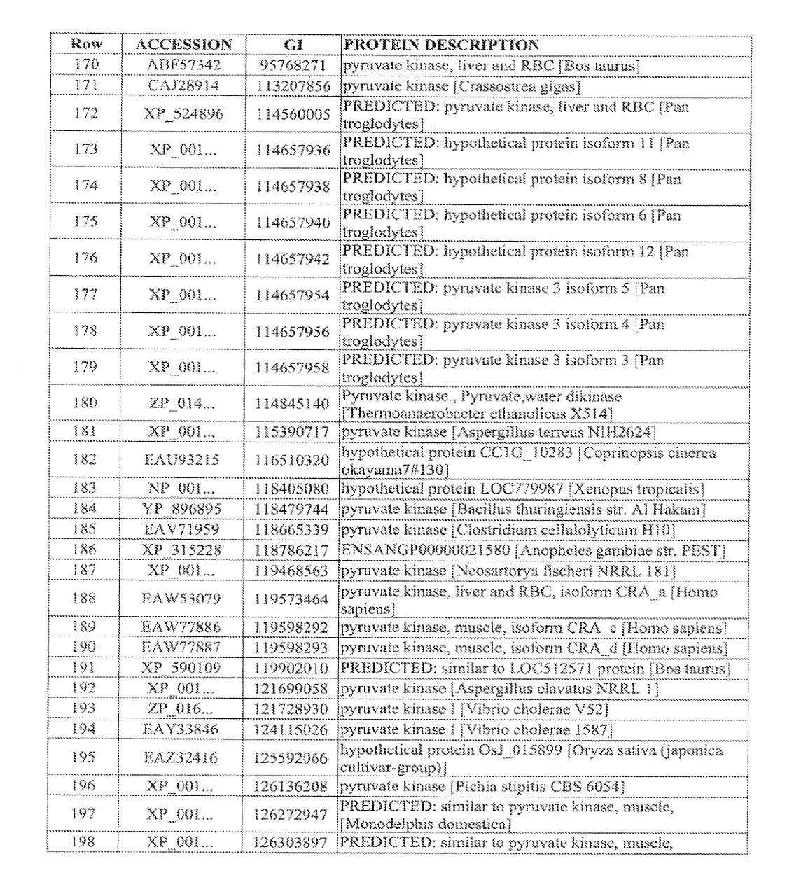

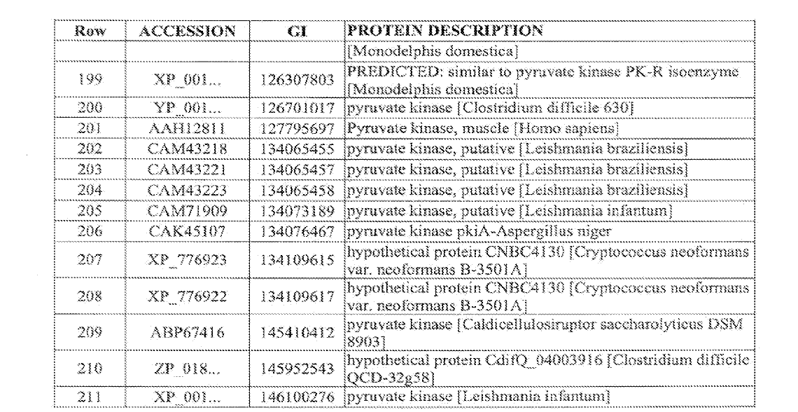

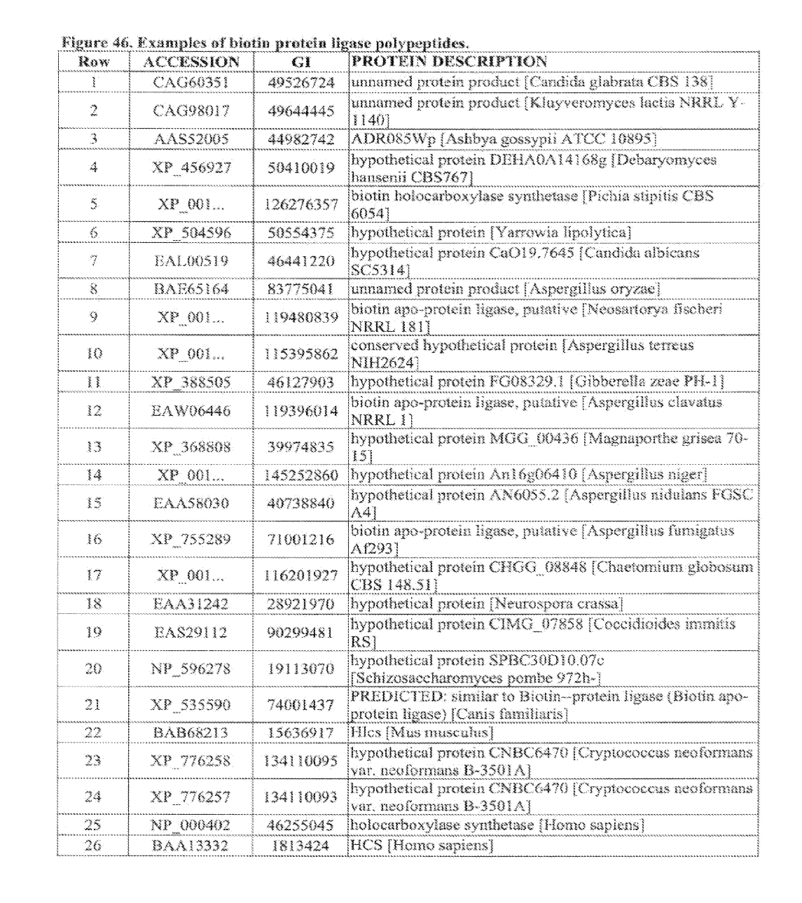

Biotin protein ligase (BPL) polypeptides: Biotin protein ligase (BPL) polypeptides catalyze the site-specific and ATP-dependent covalent transfer of biotin to the lysine side chain of the recognition sequence of an acceptor polypeptide. Acceptor polypeptides include, but are not limited to, pyruvate carboxylase polypeptides. In many instances there is a single BPL polypeptide activity in a given source organism. In some cases, a BPL polypeptide also catalyzes the biotinylation of heterologous polypeptides that are expressed in a host system. Certain BPL polypeptides are multi-functional proteins. In some embodiments, such multi-functional BPL polypeptides have functional domains that are involved in transcriptional repression. To give but one example, the BirA BPL polypeptide from E. coli has a functional domain that is incolved in transcriptional repression. Examples of biotin protein ligase (BPL) polypeptides are represented by the Genbank GI numbers in FIG. 46 and the BPL polypeptides in FIG. 29. In some cases, a biotin protein ligase (BPL) polypeptide has an amino acid sequence that is at least about 75%, 85%, 90%, 95% or 100% identical to that of a polypeptide represented by the Genbank GI numbers in FIG. 46 or a BPL polypeptide in FIG. 29.

C4-dicarboxylic acid biosynthetic polypeptides: “C4-dicarboxylic acid biosynthetic polypeptides” are proteins of primary metabolism, which are not “anaplerotic polypeptides”, whose expression and/or activity can be modified to promote the production of one or more C4-dicarboxylic acids. C4-dicarboxylic acid biosynthetic polypeptides include, but are not limited to ATP-citrate lyase polypeptides, fumarase polypeptides, fumarate reductase polypeptides, isocitrate lyase polypeptides, malate dehydrogenase polypeptides, malate synthase polypeptides, malic enzyme polypeptides, and/or succinate dehydrogenase polypeptides. C4-dicarboxylic acid biosynthetic polypeptides are represented by the ATP-citrate lyase polypeptides, fumarase polypeptides, fumarate reductase polypeptides, isocitrate lyase polypeptides, malate dehydrogenase polypeptides, malate synthase polypeptides, malic enzyme polypeptides, and succinate dehydrogenase polypeptides in FIG. 29; polypeptides that have at least 99%, 98%, 97%, 96%, 95%, 94%, 93%, 92%, 91%, 90%, 85%, 80%, 75% identity to the ATP-citrate lyase polypeptides, fumarase polypeptides, fumarate reductase polypeptides, isocitrate lyase polypeptides, malate dehydrogenase polypeptides, malate synthase polypeptides, malic enzyme polypeptides, and succinate dehydrogenase polypeptides in FIG. 29; polypeptides represented by the Genbank GI numbers in FIGS. 34, 37, 38, 41a, 41b, 42, 43, 47, and 49; and polypeptides that have at least 99%, 98%, 97%, 96%, 95%, 94%, 93%, 92%, 91%, 90%, 85%, 80%, 75% identity to a polypeptide represented by the Genbank GI numbers in FIGS. 34, 37, 38, 41a, 41b, 42, 43, 47, and 49.

C4-dicarboxylic acid derivatives: Succinic acid, malic acid and other four carbon (C4)-dicarboxylic acids are building blocks for numerous applications including surfactants, solvents, fibers, and biodegradable polymers (see Zeikus et al. (1999) Appl Microbiol Biotechnol 51: 545-552 which is hereby incorporated by reference in its entirety). Hydroxybutyrolactone and hydroxysuccinate derivatives are particular derivatives of malic acid that are of considerable commercial interest. Additional commodity chemicals that can be produced from malic acid or other C4-dicarboxylic acids (e.g. fumaric acid, succinic acid, maleic acid) include adipic acid, tetrahydrofuran (THF), butane diol (e.g. 1,4-butanediol), γ-butyrolactone, maleic anhydride, pyrrolidinones (e.g. N-methyl-2-Pyrrolidone), esters, linear aliphatic esters, diamines, 4,4-Bionelle, hydroxybutyric acid, dibasic ester (DBE), succindiamide, 1,4-diaminobutane, succinonitrile, and unsaturated succinate derivatives. The derivatives may be produced by any number of processes including physical treatments, fermentation, biocatalysis, chemical transformation and combinations thereof.

Carbonic anhydrase (CA) polypeptides: “Carbonic anhydrase (CA) polypeptides” are zinc metalloenzymes enzymes that catalyze the reaction CO₂H₂O [symbol] H₂CO₃ (EC 4.2.1.1). At least three distinct classes of CA polypeptides (designated α, β and γ) exist that have no significant sequence identity. Mammalian CA polypeptides belong to the a class, together with limited representatives from Bacteria and Archaea. β class CA polypeptides includes enzymes from the chloroplasts of both monocotyledonous and dicotyledonous plants as well as enzymes from phylogenetically diverse species from the Archaea and Bacteria domains. The CA polypeptide from the methanoarchaeon Methanosarcina thermophila is a representative of γ class CA polypeptides. Distinct CA polypeptide activities have been detected extracellularly, in the cytosol, and within multiple organelles. CA polypeptides are involved in several important physiological functions, including transport of CO₂/HCO₃⁻, pH and CO₂ homeostasis, biosynthetic reactions, such as anaplerosis and gluconeogenesis, and CO₂ fixation (in plants and algae). Examples of carbonic anhydrase (CA) polypeptides are represented by the Genbank GI numbers in FIG. 40 and the CA polypeptides in FIG. 29. In some cases, a carbonic anhydrase (CA) polypeptide has an amino acid sequence that is at least about 75%, 85%, 90%, 95% or 100% identical to that of a polypeptide represented by the Genbank GI numbers in FIG. 40 or a CA polypeptide in FIG. 29.

Codon: As is known in the art, the term “codon” refers to a sequence of three nucleotides that specify a particular amino acid.

DNA ligase: The term “DNA ligase” refers to an enzyme that covalently joins two pieces of double-stranded DNA.

Electroporation: The term “electroporation” refers to a method of introducing foreign DNA into cells that uses a brief, high voltage DC charge to permeabilize the host cells, causing them to take up extra-chromosomal DNA.

Endonuclease: The term “endonuclease” refers to an enzyme that hydrolyzes double stranded DNA at internal locations.

Expression: The term “expression” refers to the production of a gene product (i.e., RNA or protein). For example, “expression” includes transcription of a gene to produce a corresponding mRNA, and translation of such an mRNA to produce the corresponding peptide, polypeptide, or protein.

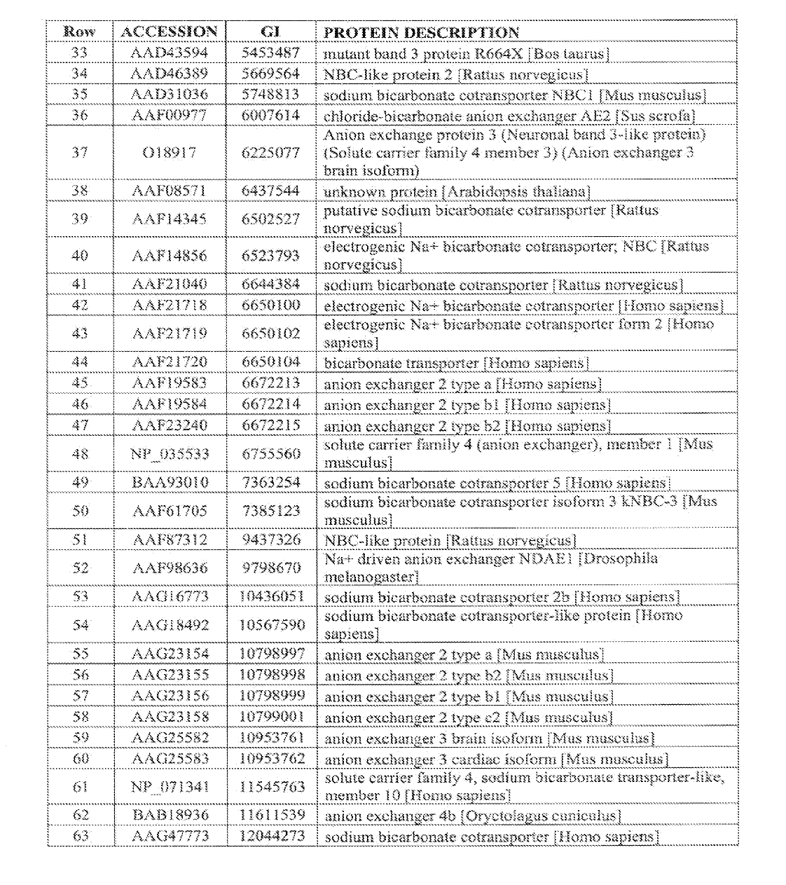

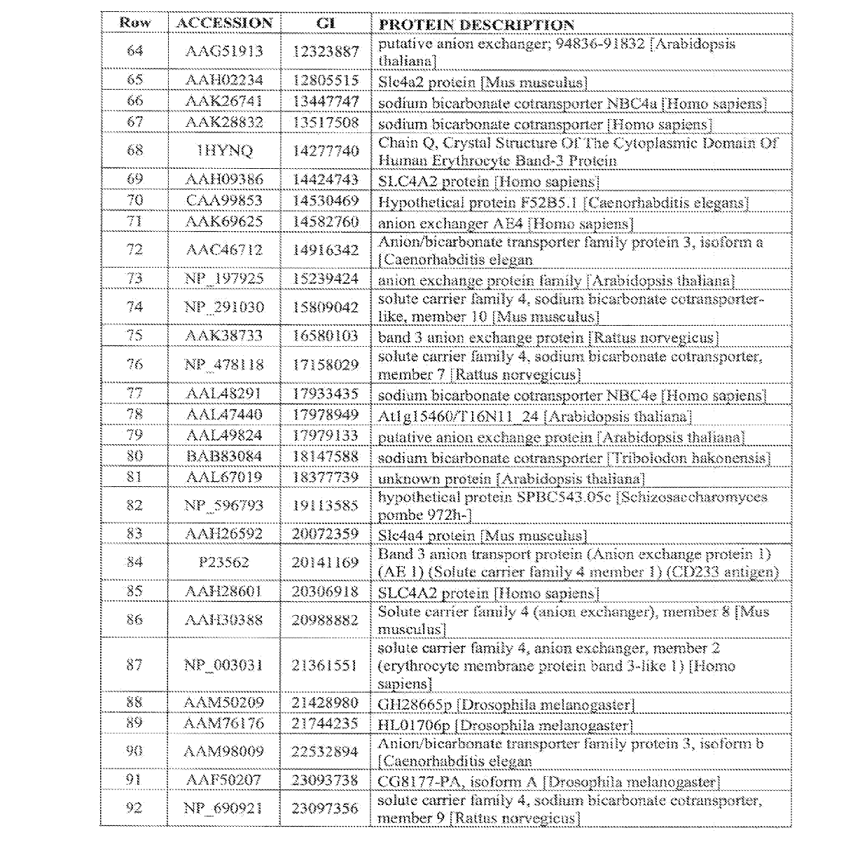

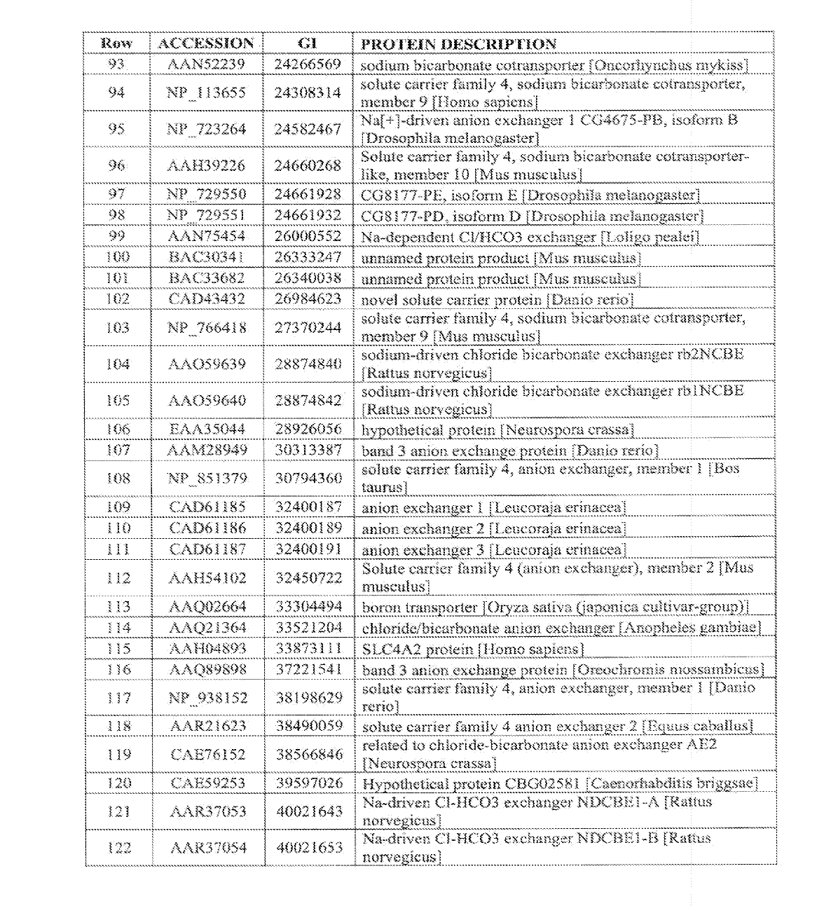

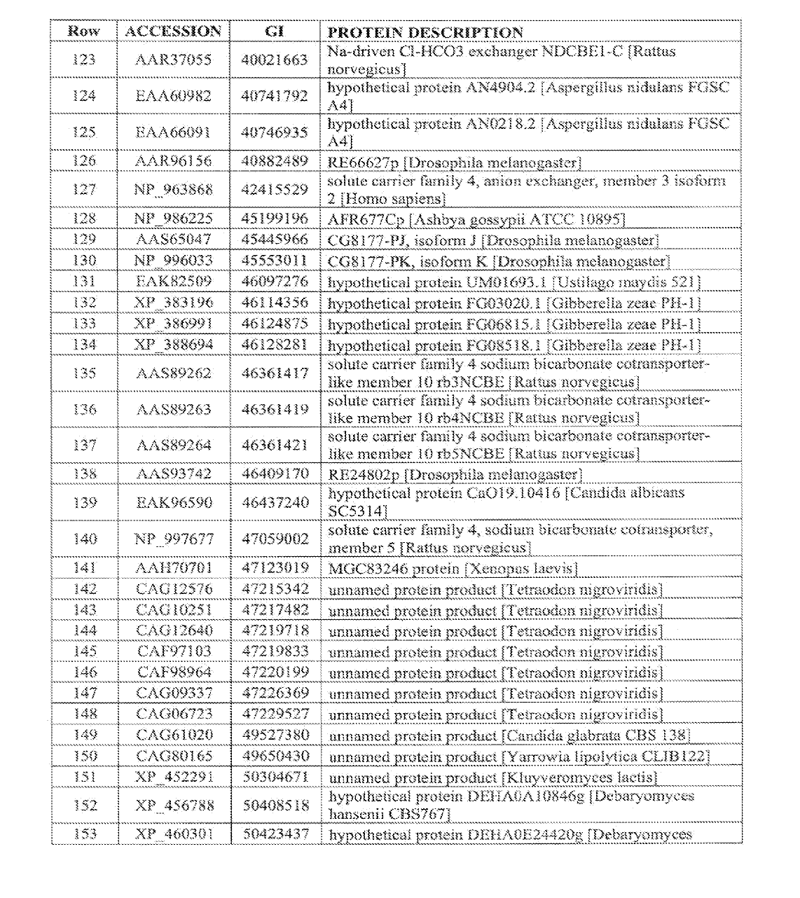

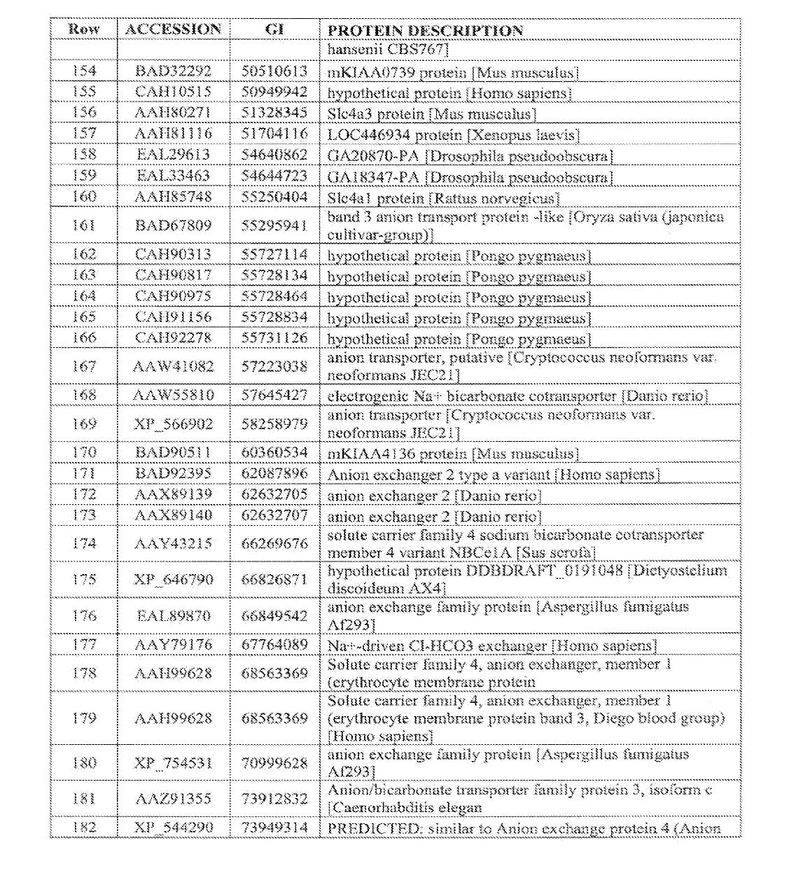

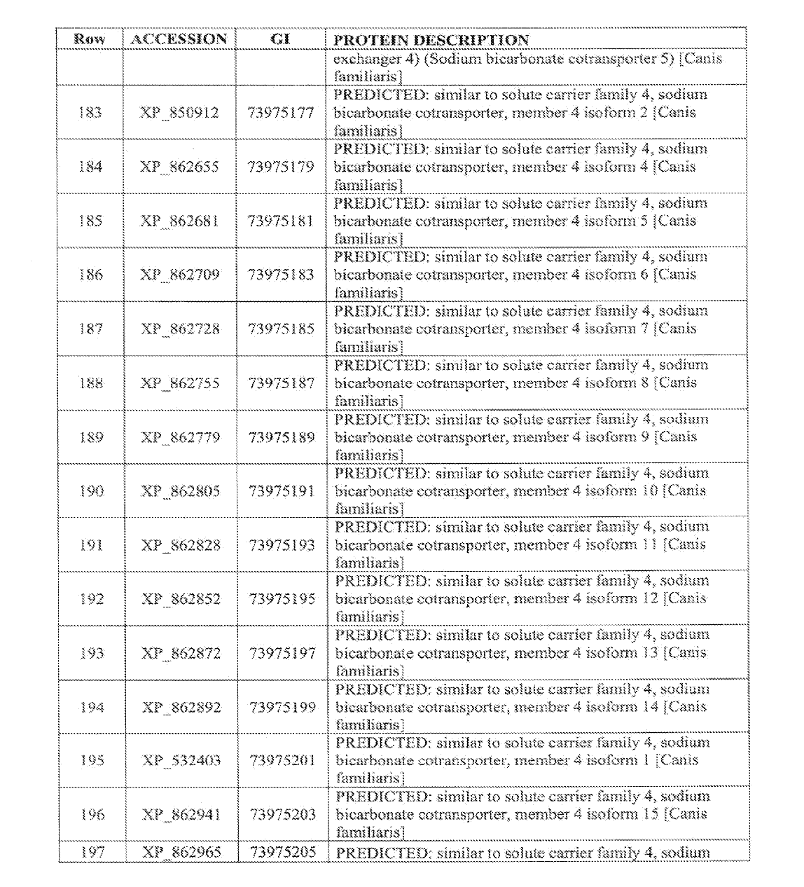

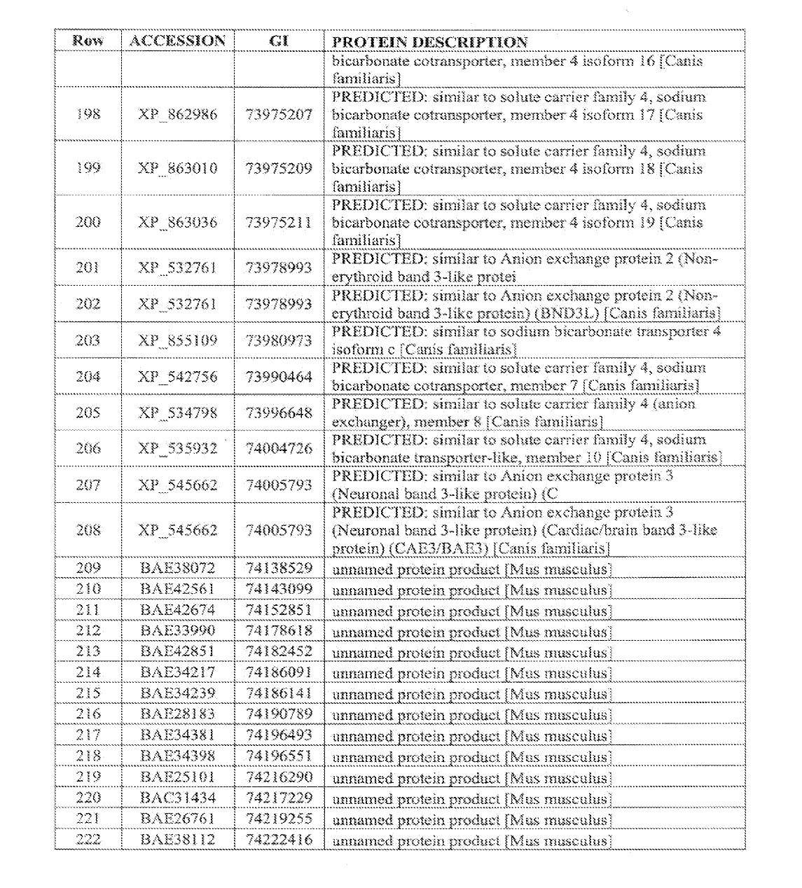

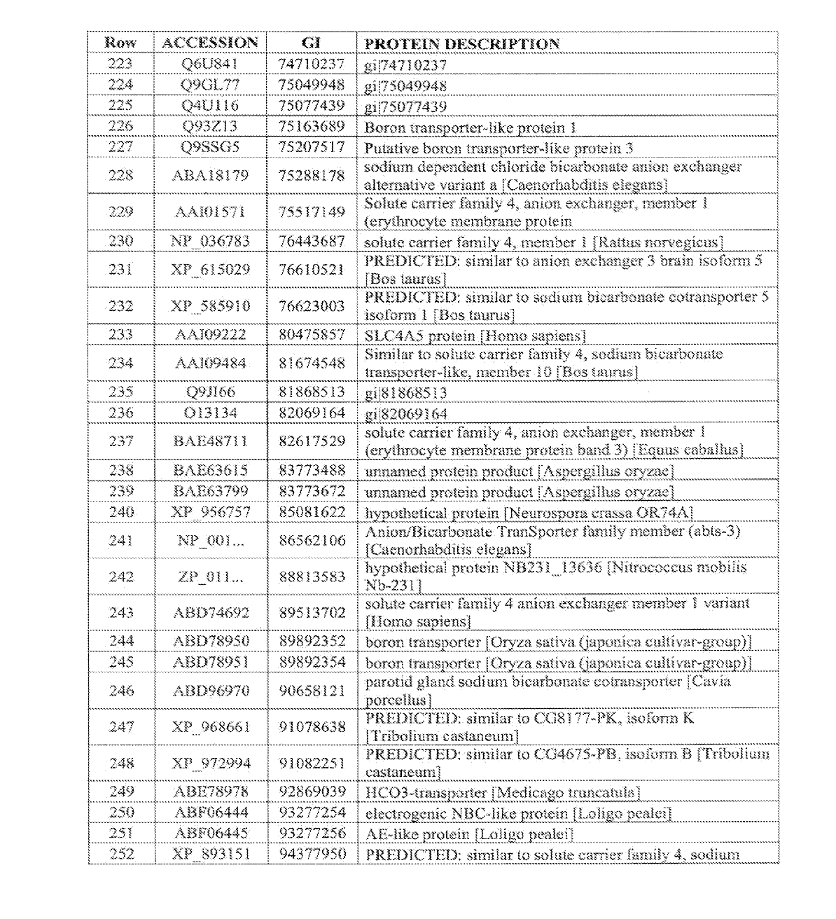

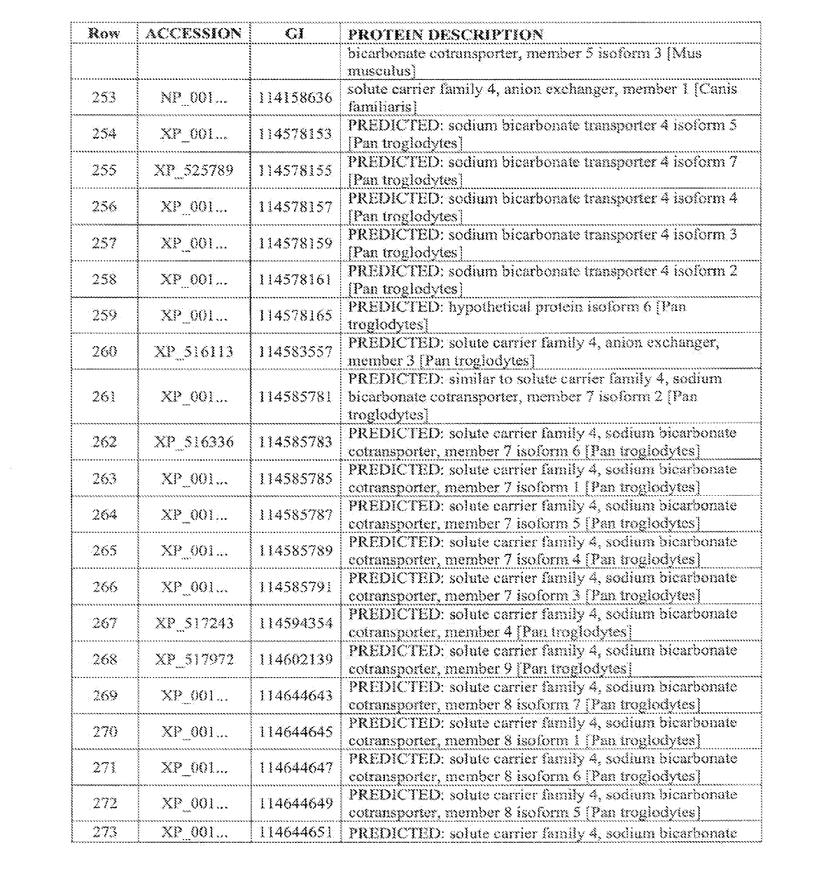

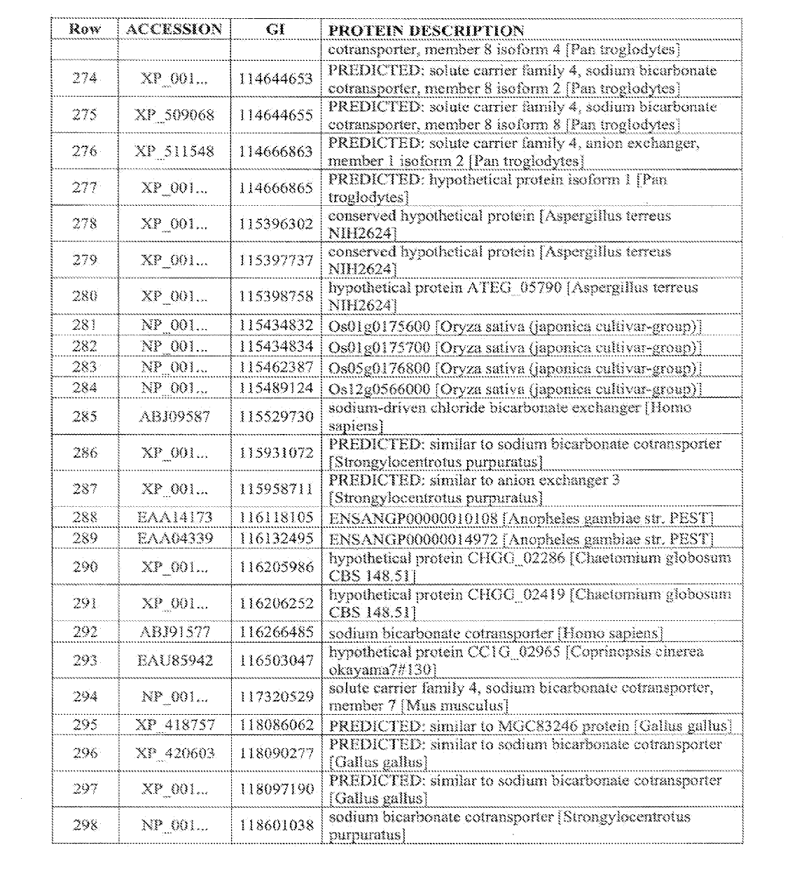

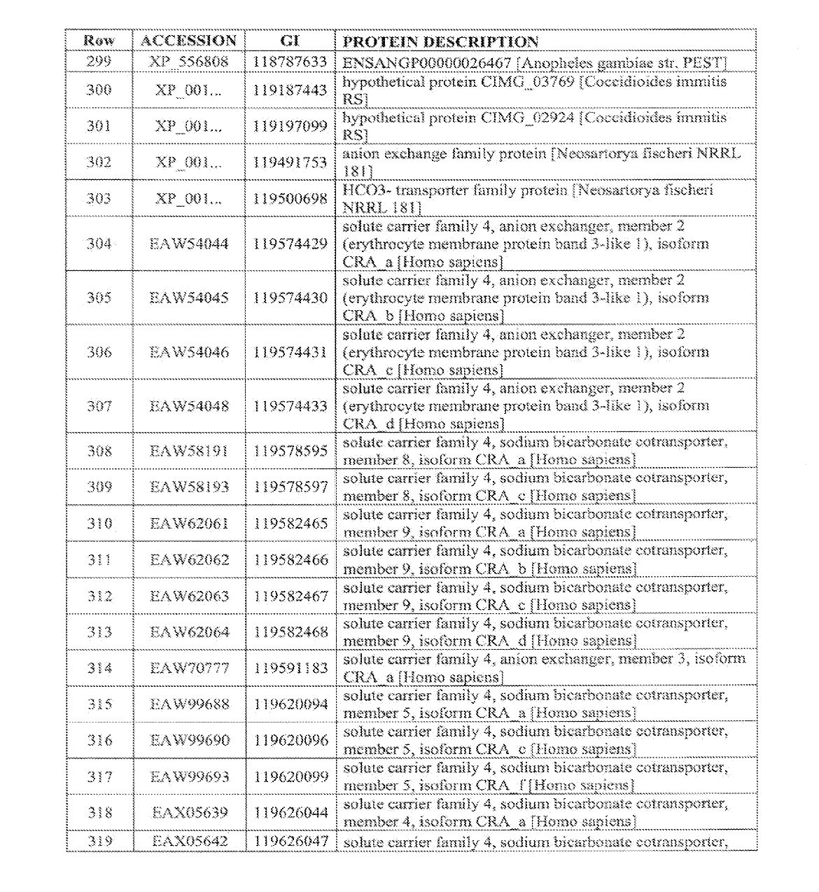

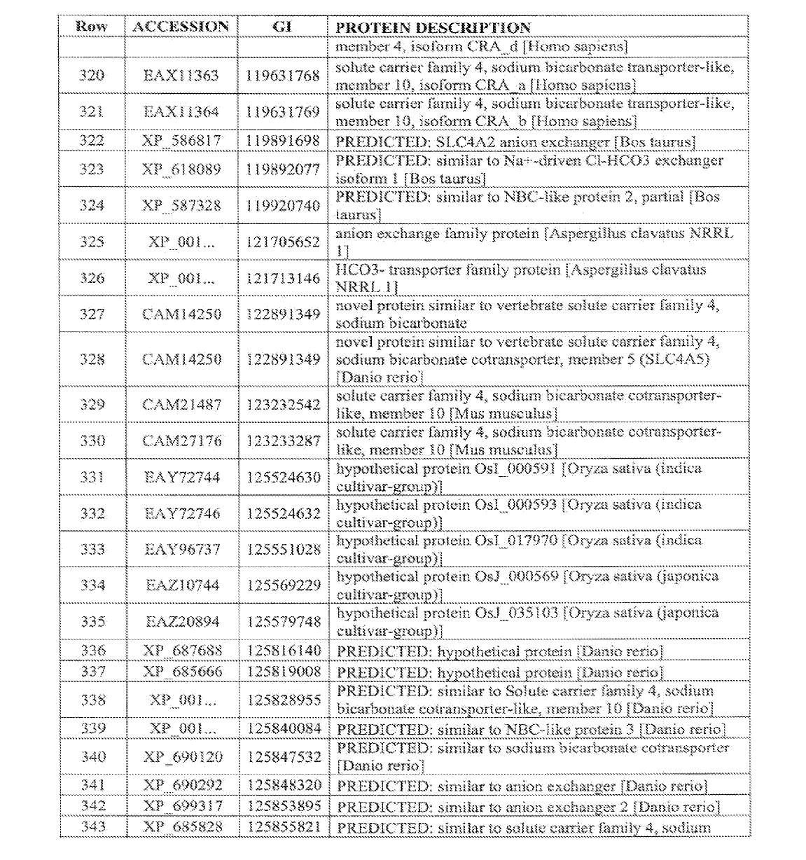

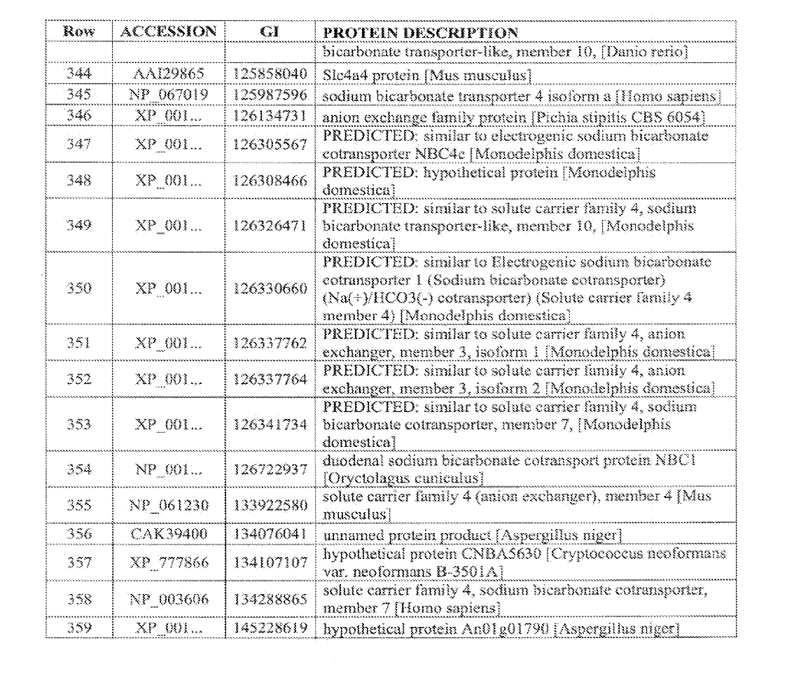

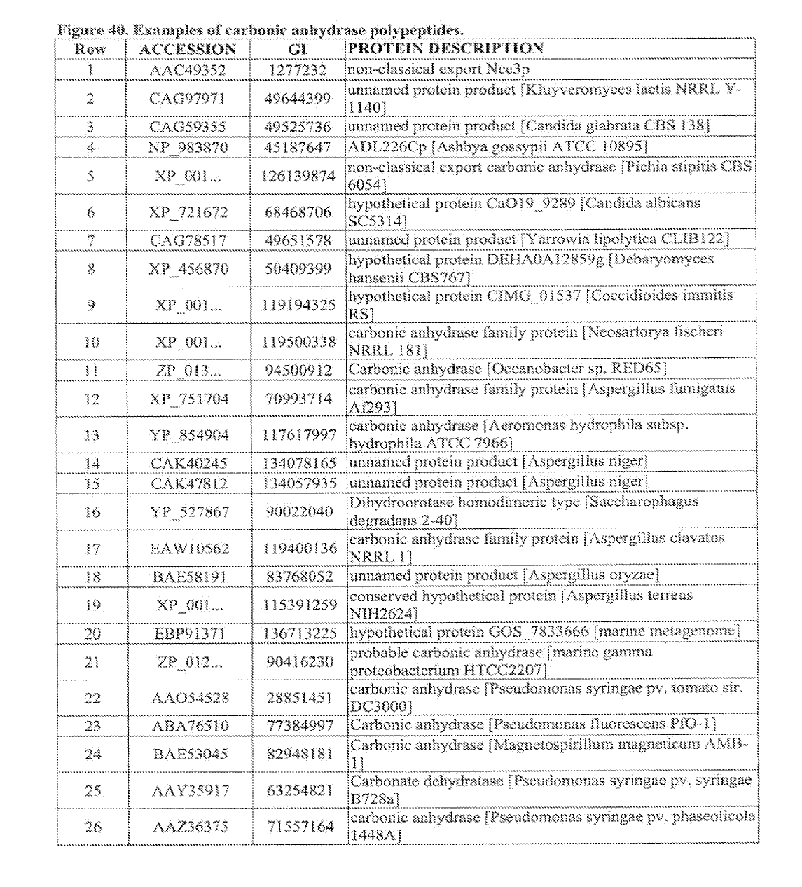

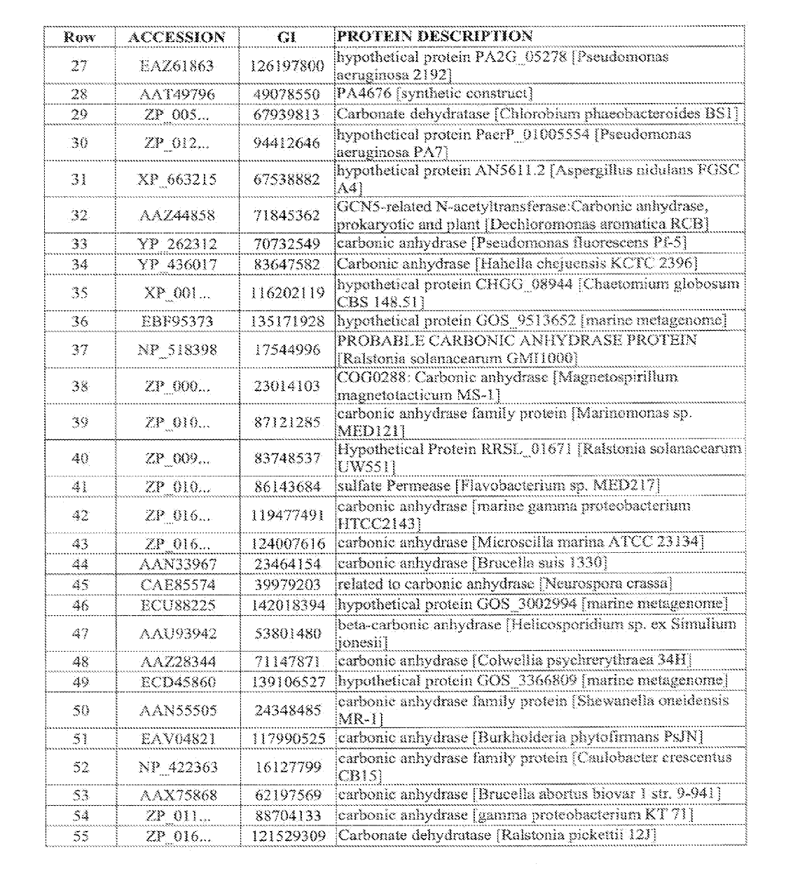

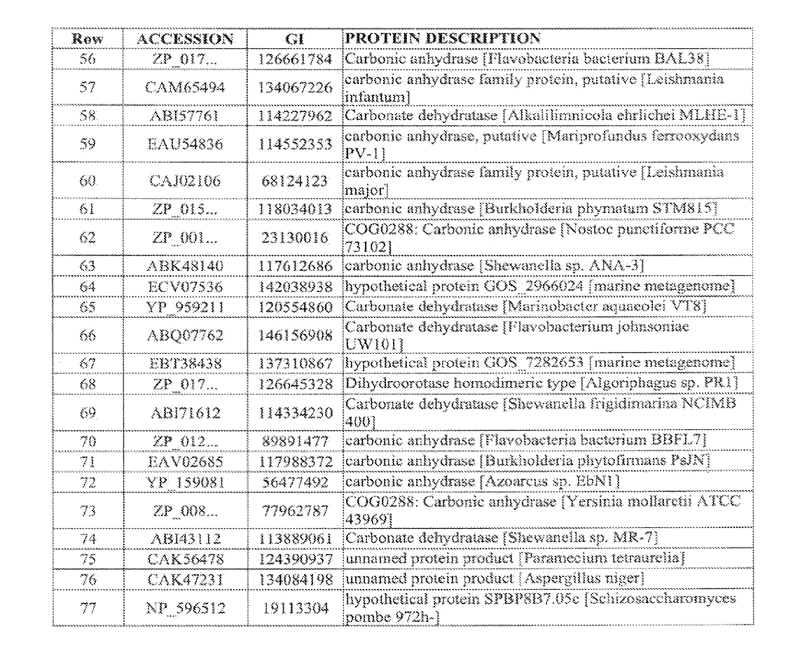

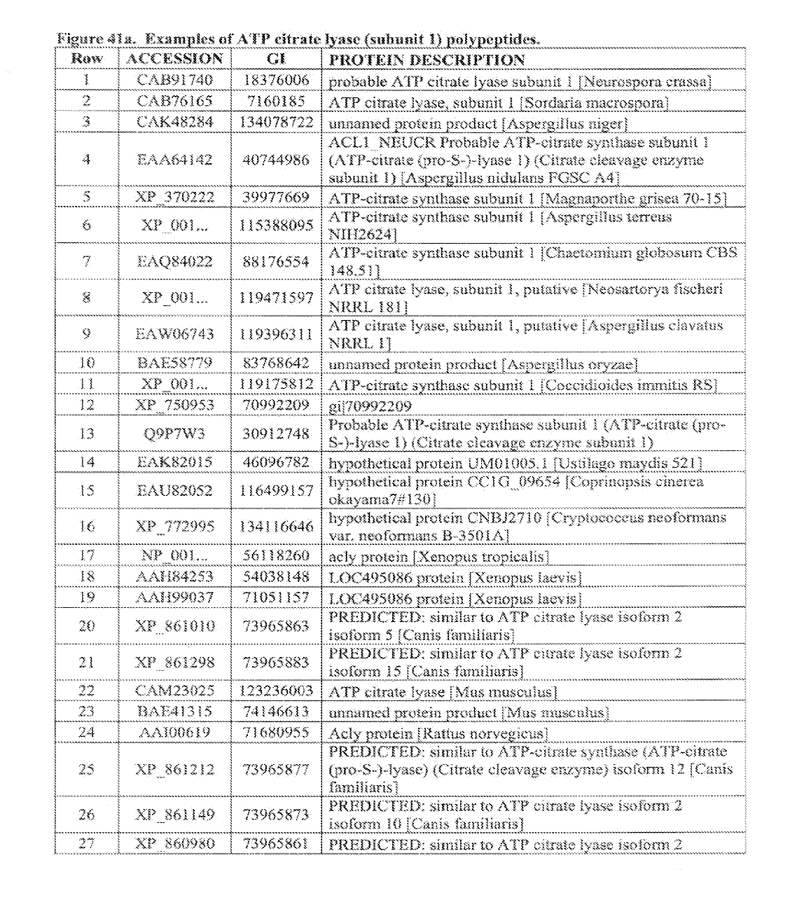

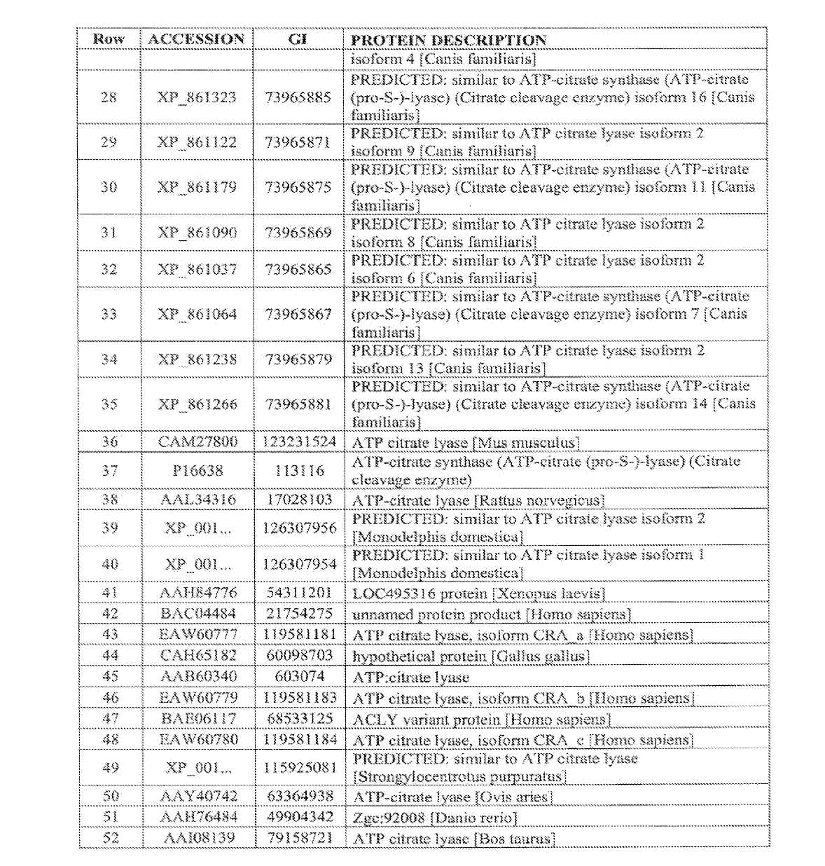

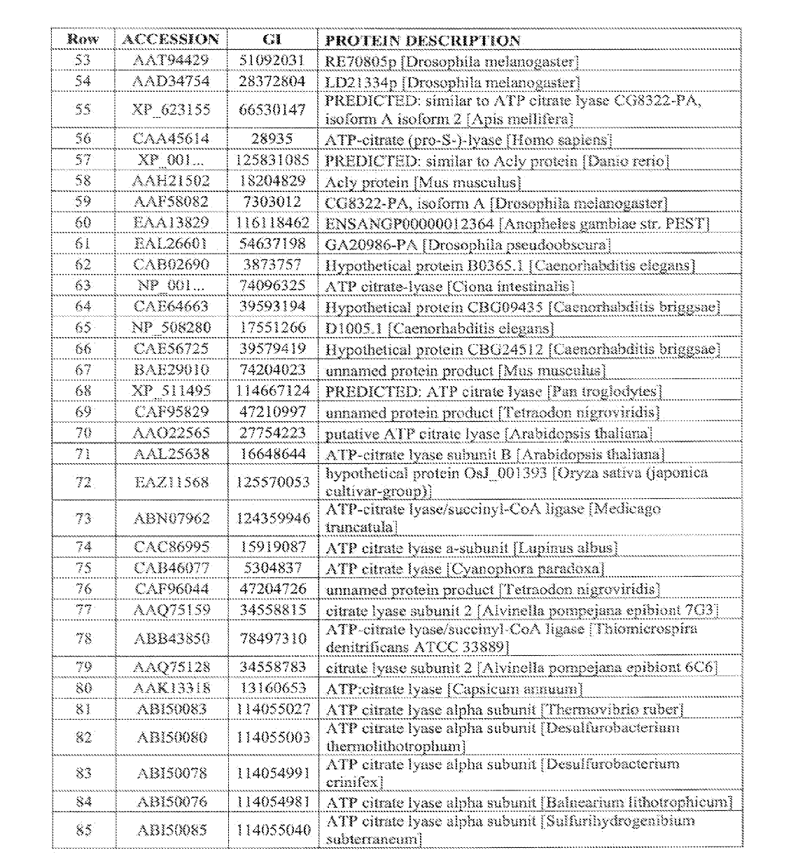

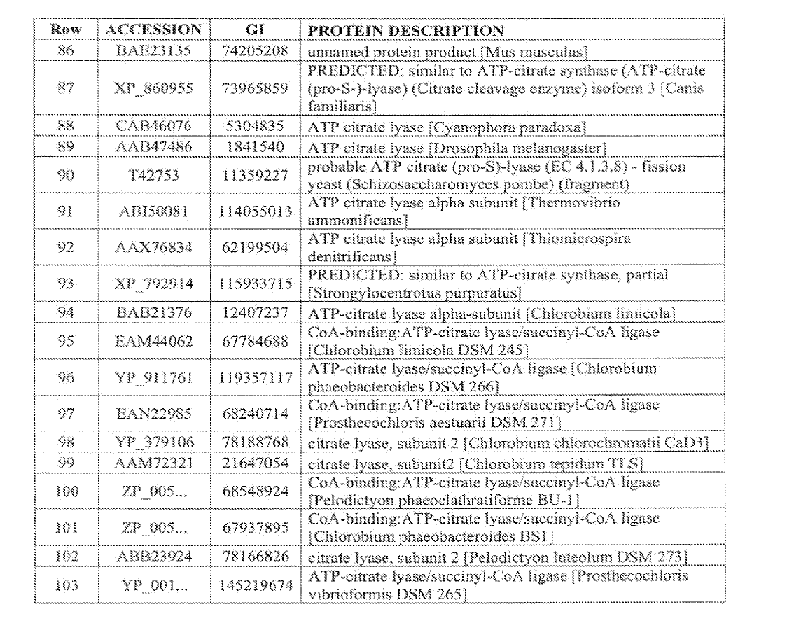

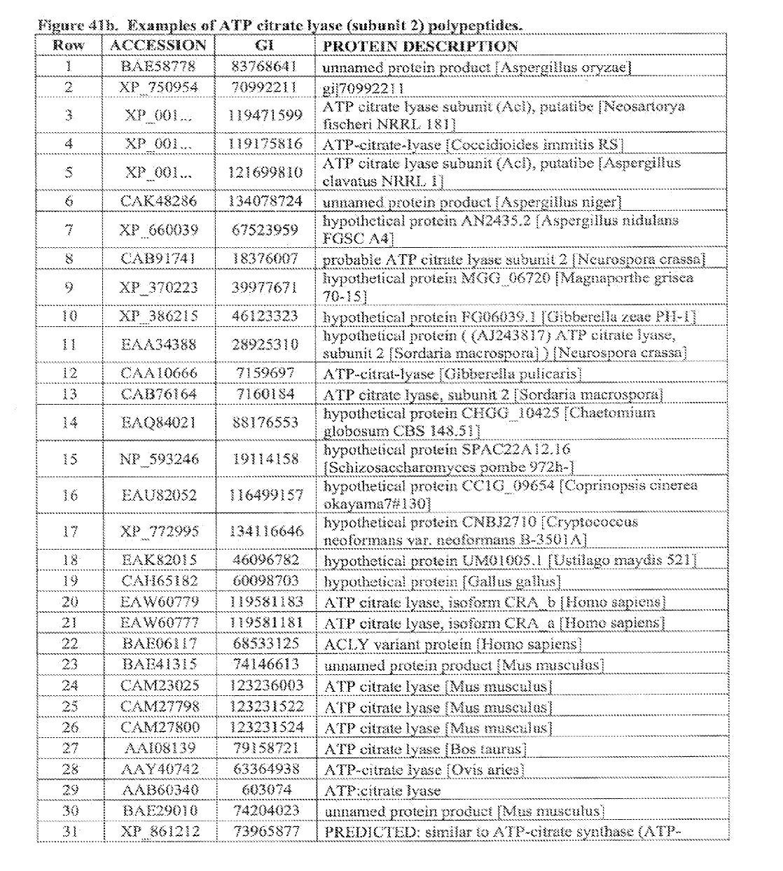

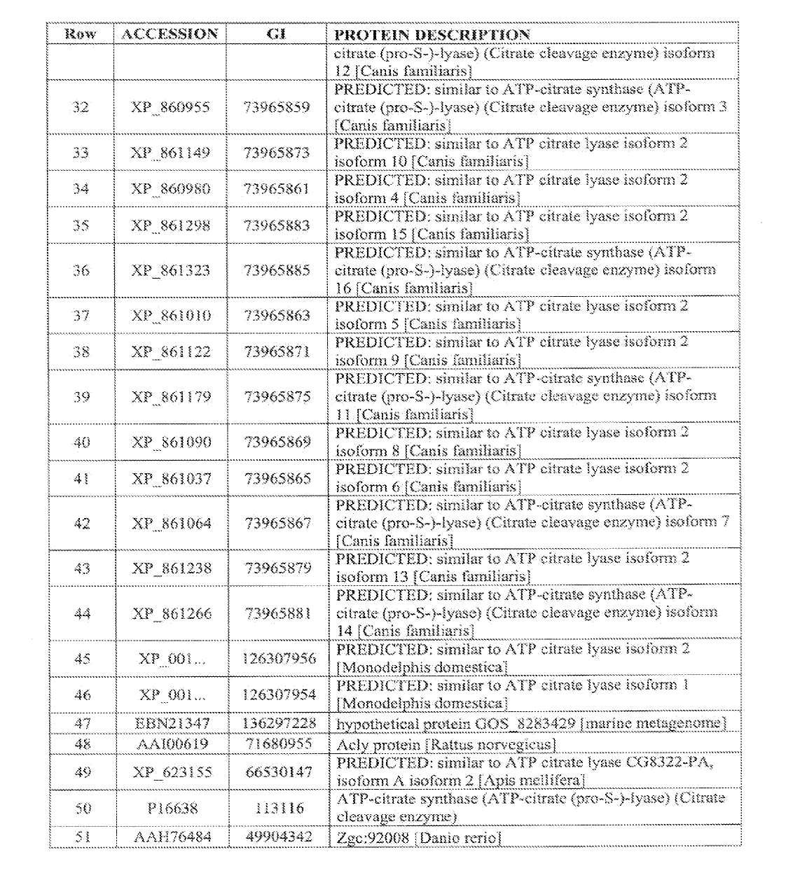

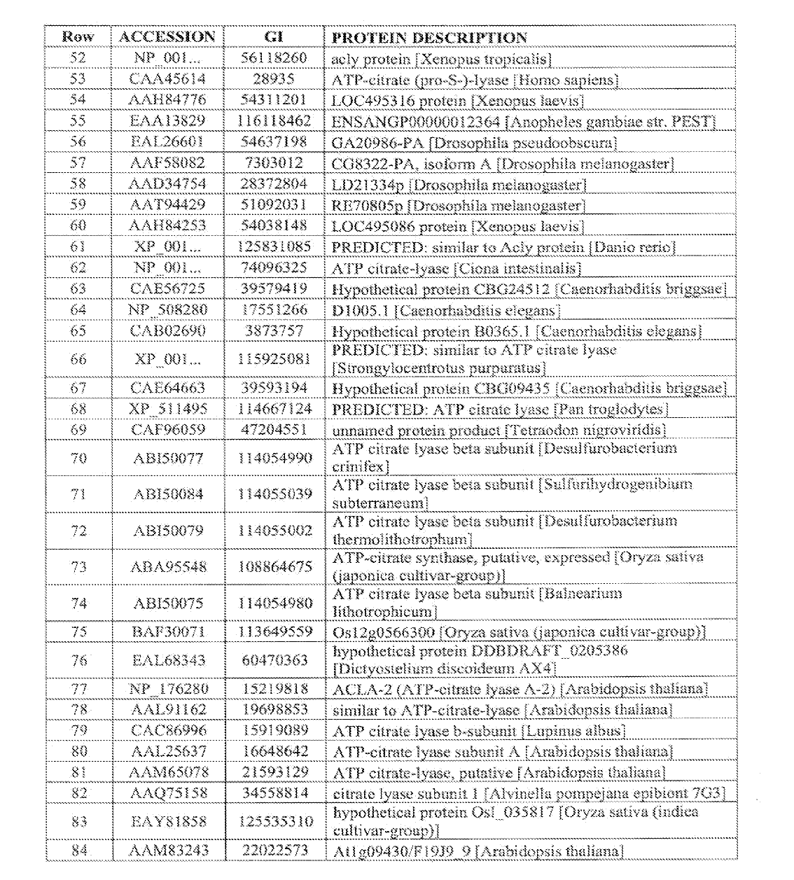

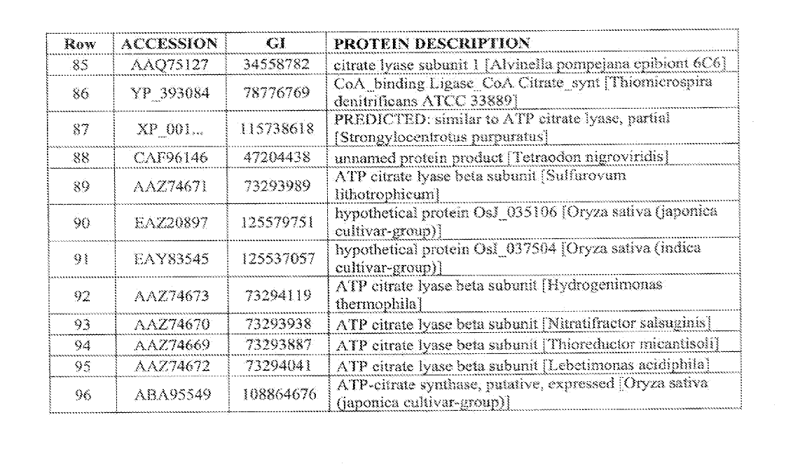

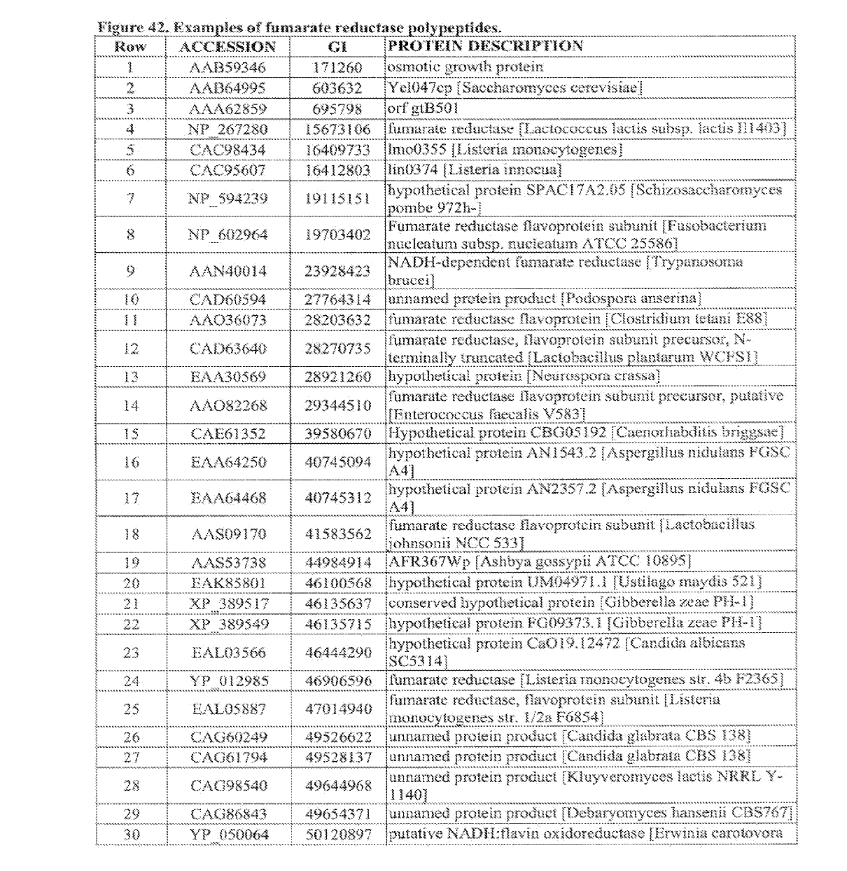

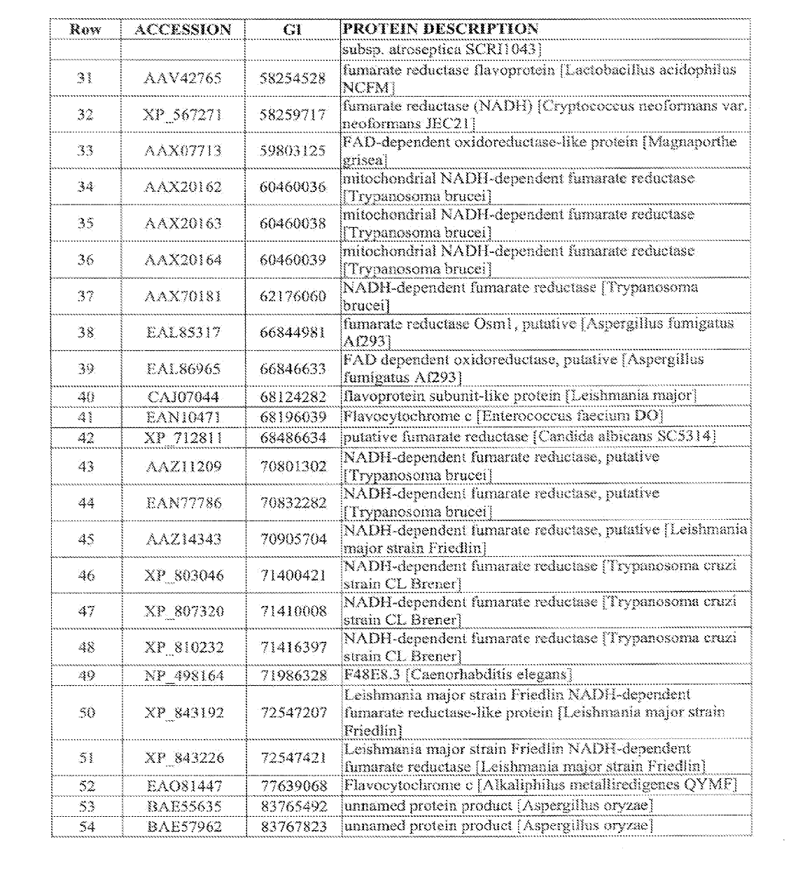

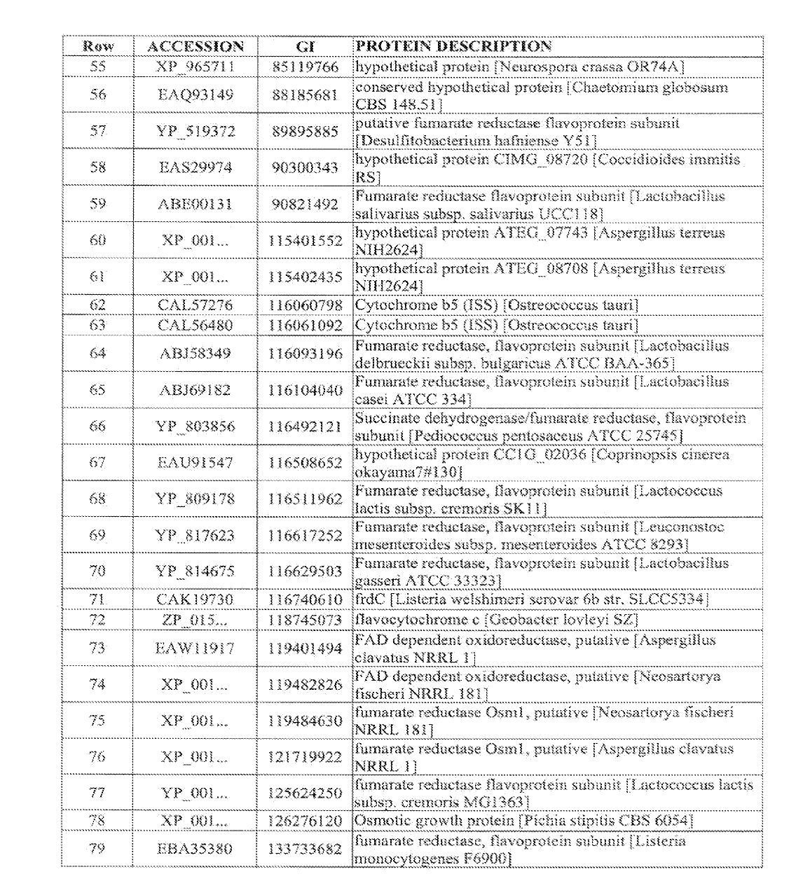

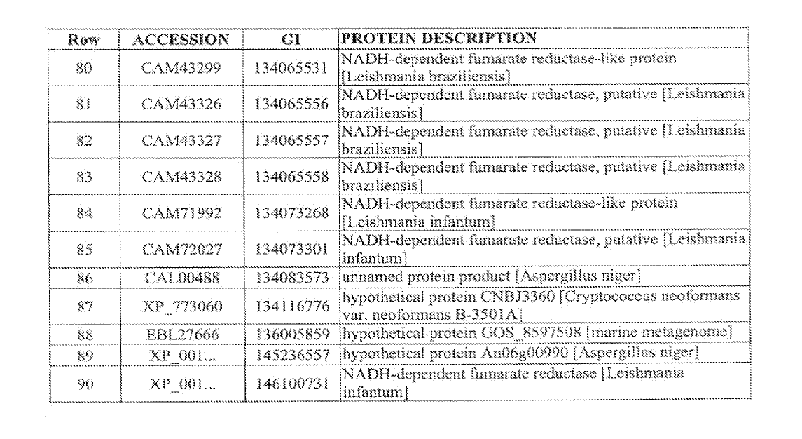

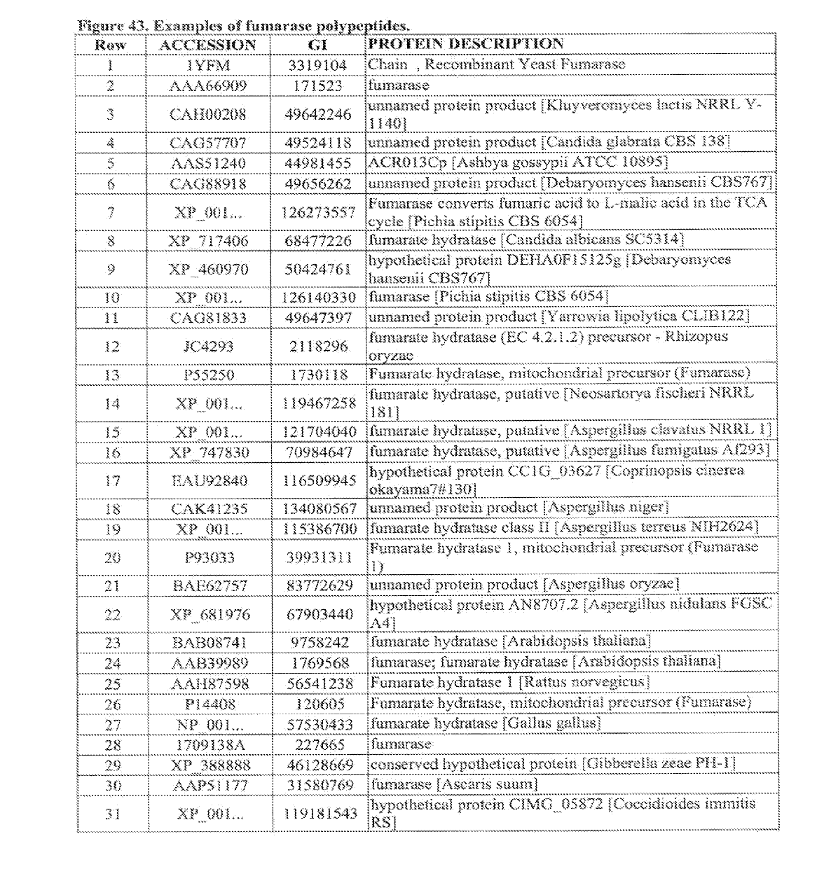

Fumarase polypeptides: “Fumarase polypeptides” are polypeptides that catalyze the reversible hydration of fumarate to malate (EC 4.2.1.2). In the mitochondrial matrix, fumarase polypeptides function in the tricarboxylic acid cycle to convert fumarate to malate. Fumarase activities often are present in the cytosol as well as the mitochondria. In S. cerevisiae, the cytosolic and mitochondrial fumarase isoenzymes are encoded by one gene, FUM1. Fumarase polypeptides are synthesized as precursors and are targeted to and processed in mitochondria prior to distribution between the cytosol and mitochondria. Deletion of the amino terminal mitochondrial-targeting sequence and signal peptide of FUM1 results in exclusive cytosolic localization. It is likely that functional FUM1 polypeptide variants that preferentially localize to the mitochondria can also be identified. Examples of fumarase polypeptides are represented by the Genbank GI numbers in FIG. 43 and the fumarase polypeptides in FIG. 29. In some cases, a fumarase polypeptide has an amino acid sequence that is at least about 75%, 85%, 90%, 95% or 100% identical to that of a polypeptide represented by the Genbank GI numbers in FIG. 43 or a fumarase polypeptide in FIG. 29.

Fumarate reductase polypeptides: “Fumarate reductase polypeptides” are a set of FAD-binding proteins that catalyze, to different extents, the interconversion of fumarate and succinate. Fumarate reductase polypeptides are generally active in anaerobic or facultative microbes that live a portion of their life cycle in a reduced oxygen environment. The S. cerevisiae fumarate reductase, similar to the flavocytochrome c from Shewanella species, is a soluble protein that binds FAD non-covalently and catalyzes the irreversible reduction of fumarate to succinate, which is required for the reoxidation of intracellular NADH under anaerobic conditions. The S. cerevisiae fumarate reductase polypeptide activities are encoded by the OSM1 (mitochondria) and FRDS1 (at least partially cytosolic) genes. A distinct class of fumarate reductases is membrane-bound, possesses covalently-linked FAD, and is more structurally related to succinate dehydrogenases; these fumarate reductase polypeptides display some extent of oxidation of succinate to fumarate. Examples of fumarate reductase polypeptides are represented by the Genbank GI numbers in FIG. 42 and the fumarate reductase polypeptides in FIG. 29. In some cases, a polypeptide has an amino acid sequence that is at least about 75%, 85%, 90%, 95% or 100% identical to that of a polypeptide represented by the Genbank GI numbers in FIG. 42 or a fumarate reductase polypeptide in FIG. 29.

Functionally linked: The phrase “functionally linked” or “operably linked” refers to a promoter or promoter region and a coding or structural sequence in such an orientation and distance that transcription of the coding or structural sequence may be directed by the promoter or promoter region.

Functionally transformed: As used herein, the term “functionally transformed” refers to a host cell that has been caused to express one or more polypeptides as described herein, such that the expressed polypeptide is functional and is active at a level higher than is observed with an otherwise identical cell (i.e., a parental cell) that has not been so transformed. In many embodiments, functional transformation involves introduction of a nucleic acid encoding the polypeptide(s) such that the polypeptide(s) is/are produced in an active form and/or appropriate location. Alternatively or additionally, in some embodiments, functional transformation involves introduction of a nucleic acid that regulates expression of such an encoding nucleic acid.

Gene: The term “gene”, as used herein, generally refers to a nucleic acid encoding a polypeptide, optionally including certain regulatory elements that may affect expression of one or more gene products (i.e., RNA or protein). A gene may be in chromosomal DNA, plasmid DNA, cDNA, synthetic DNA, or other DNA that encodes a peptide, polypeptide, protein, or RNA molecule, and may include regions flanking the coding sequence involved in the regulation of expression.

Genome: The term “genome” encompasses both the chromosomes and plasmids within a host cell. For example, encoding nucleic acids of the present invention that are introduced into host cells can be part of the genome whether they are chromosomally integrated or plasmid-localized.

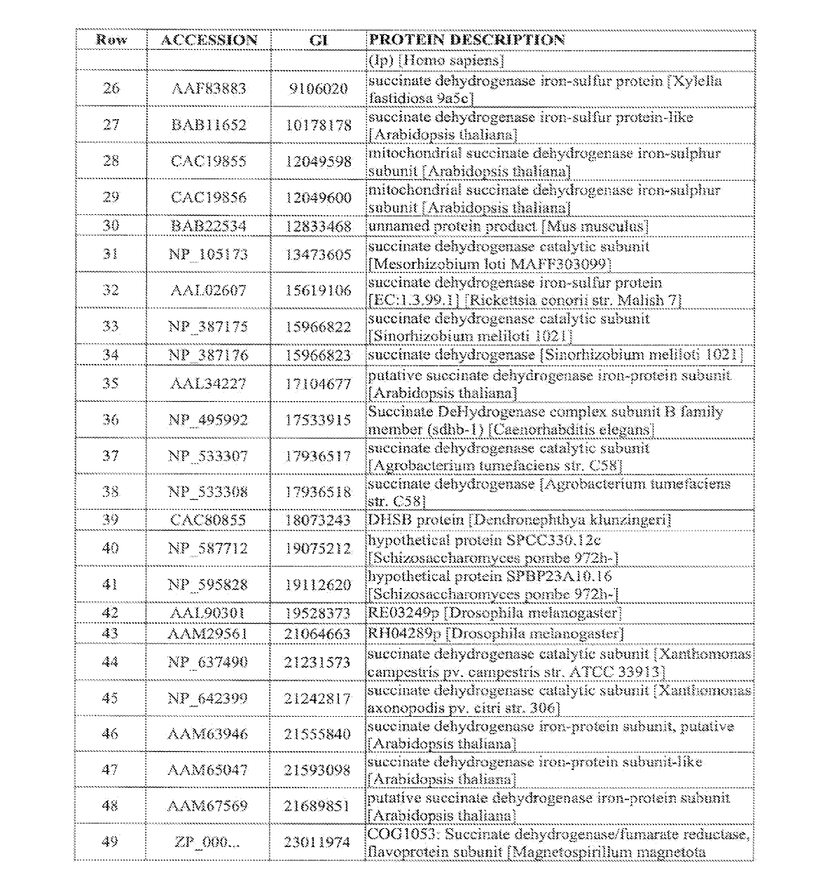

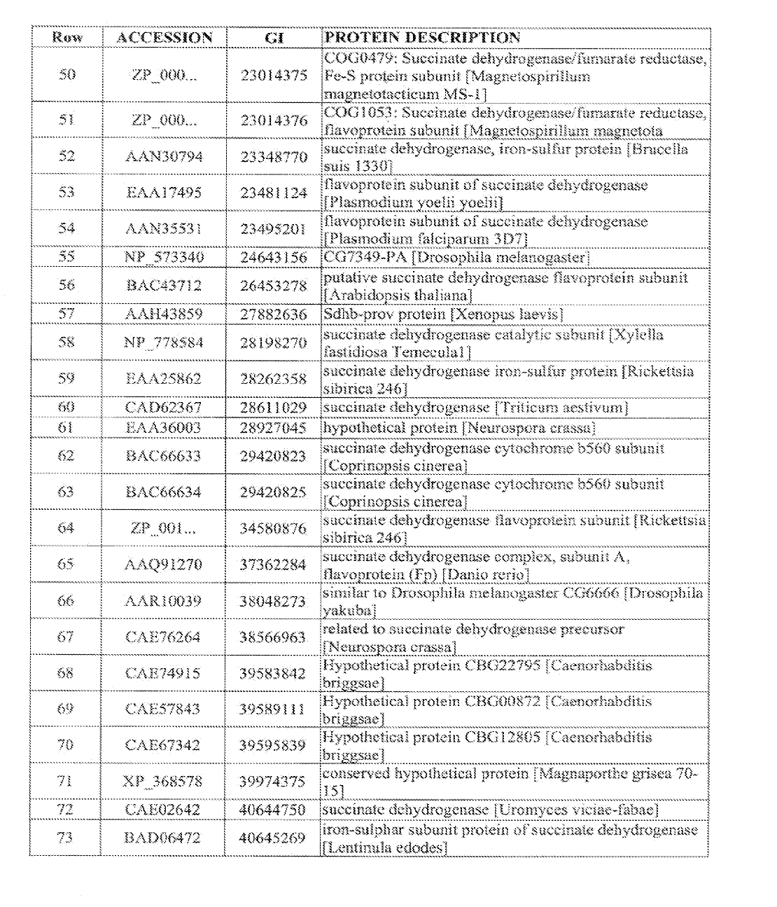

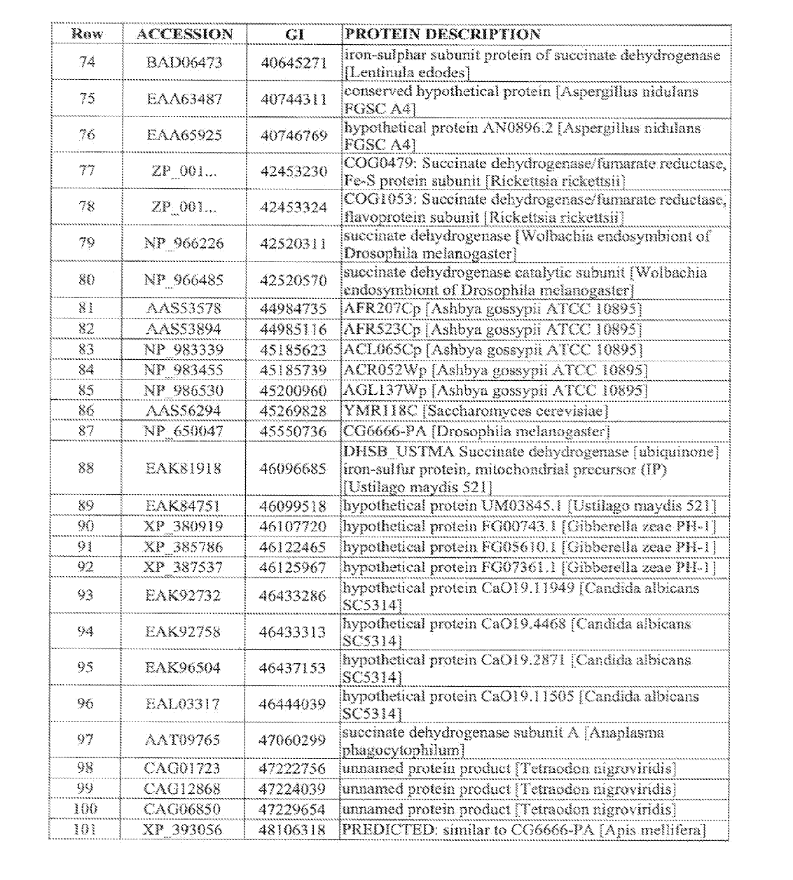

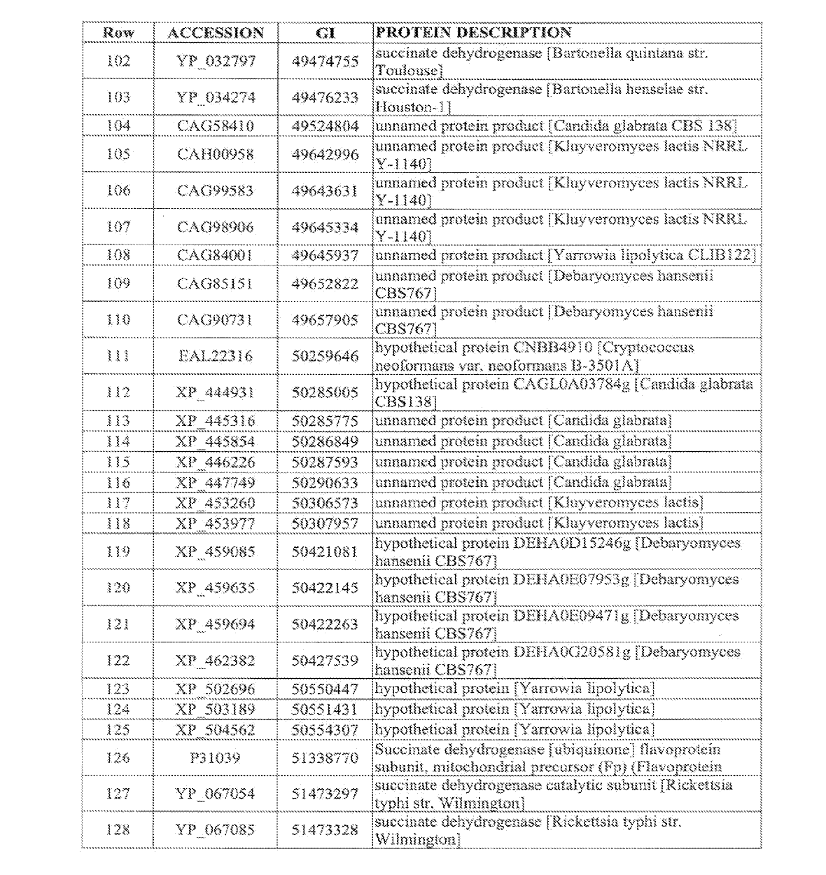

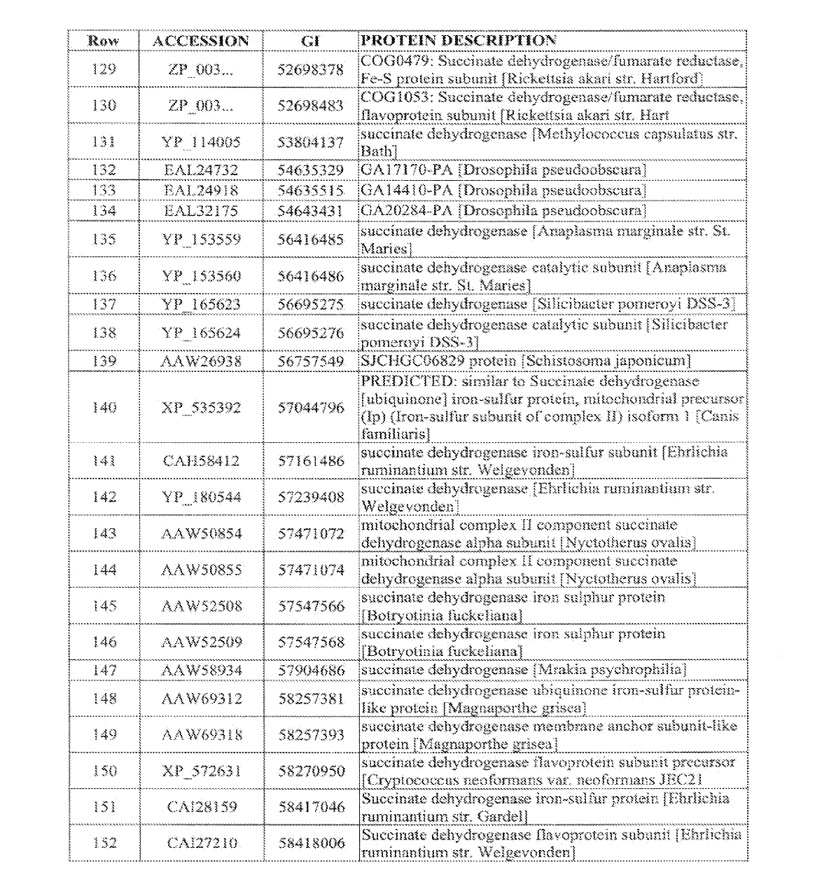

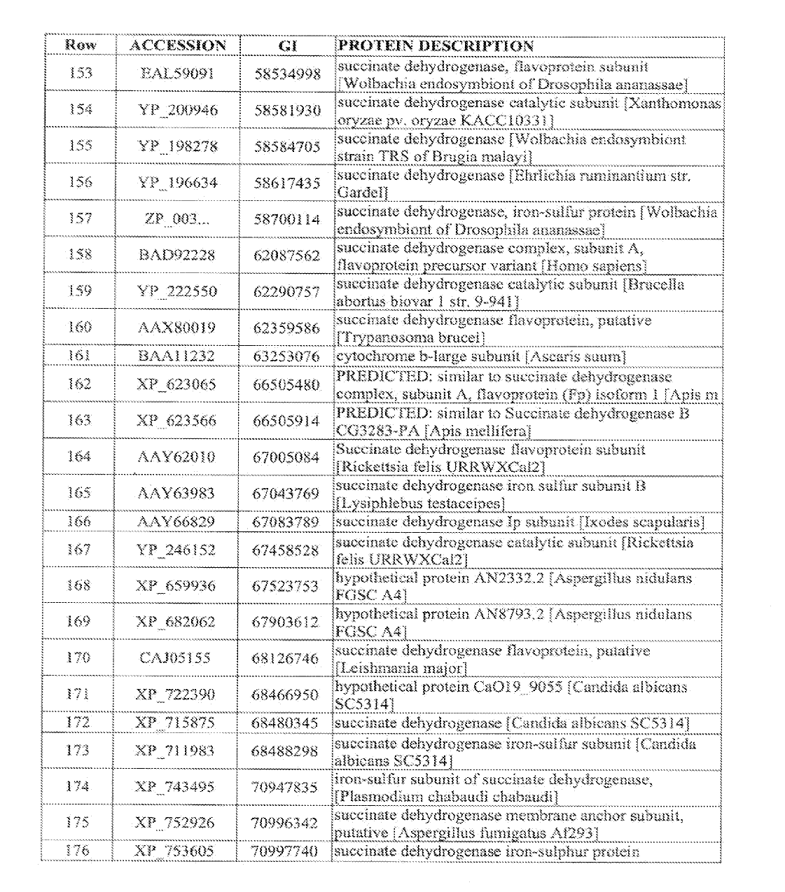

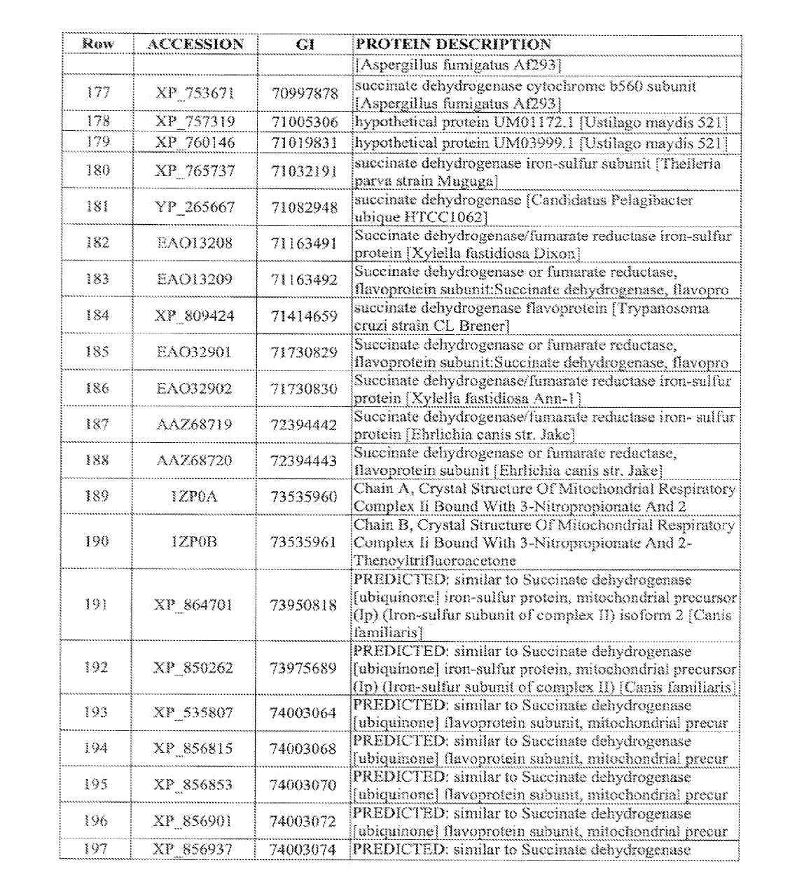

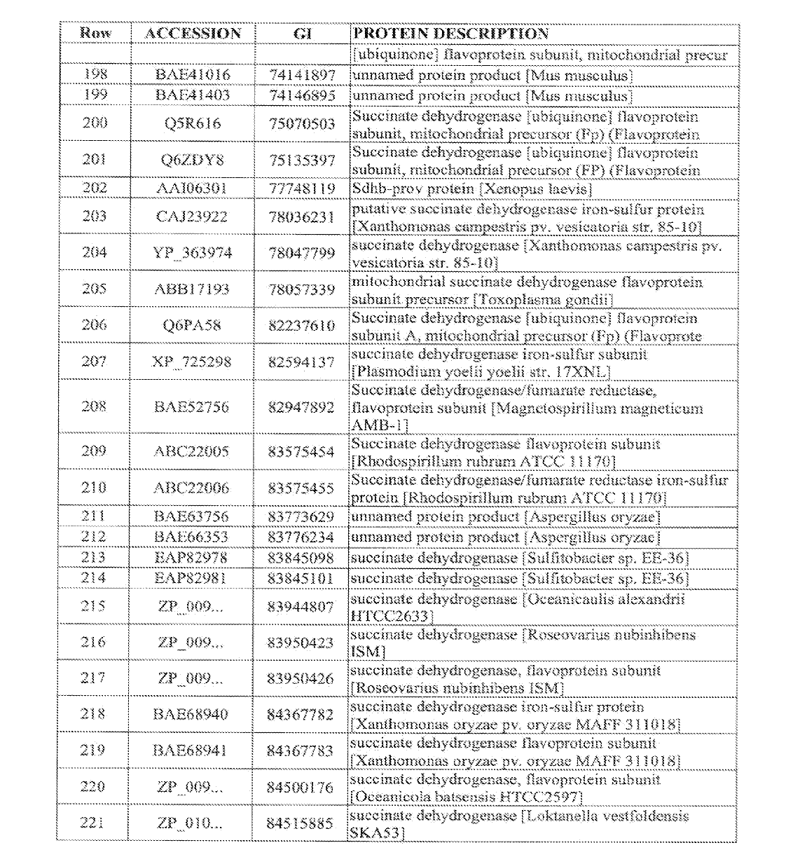

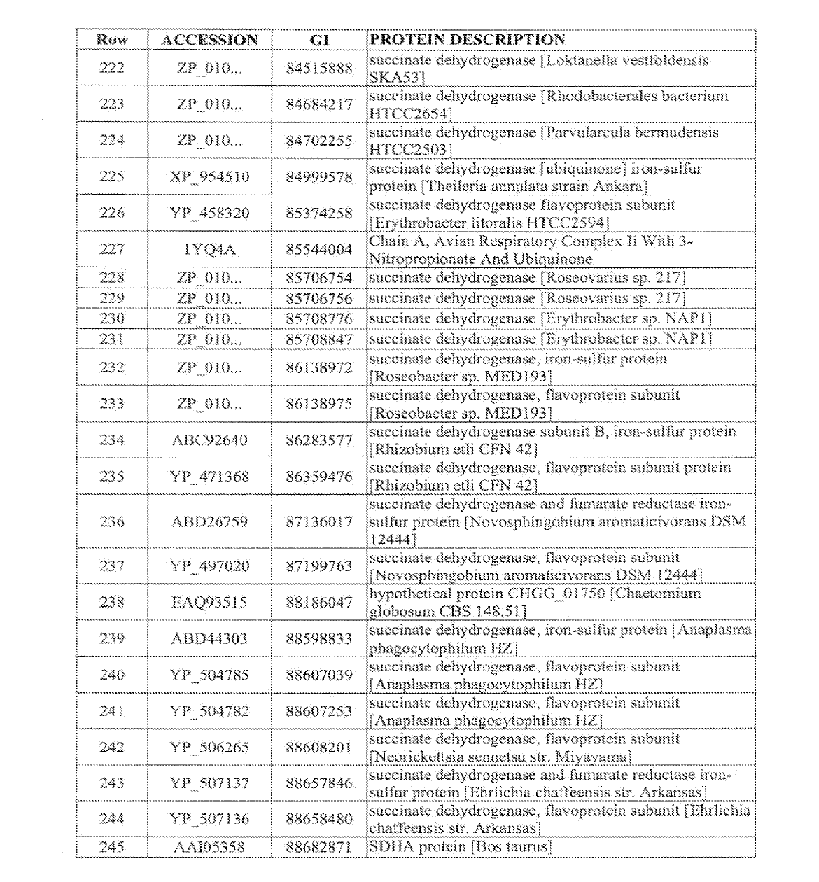

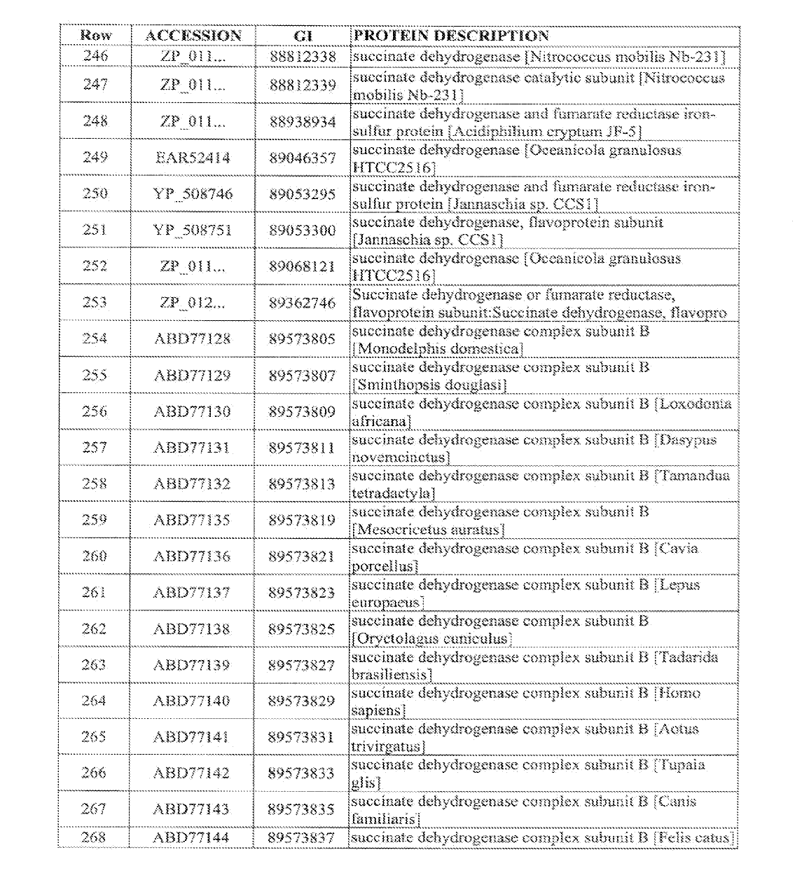

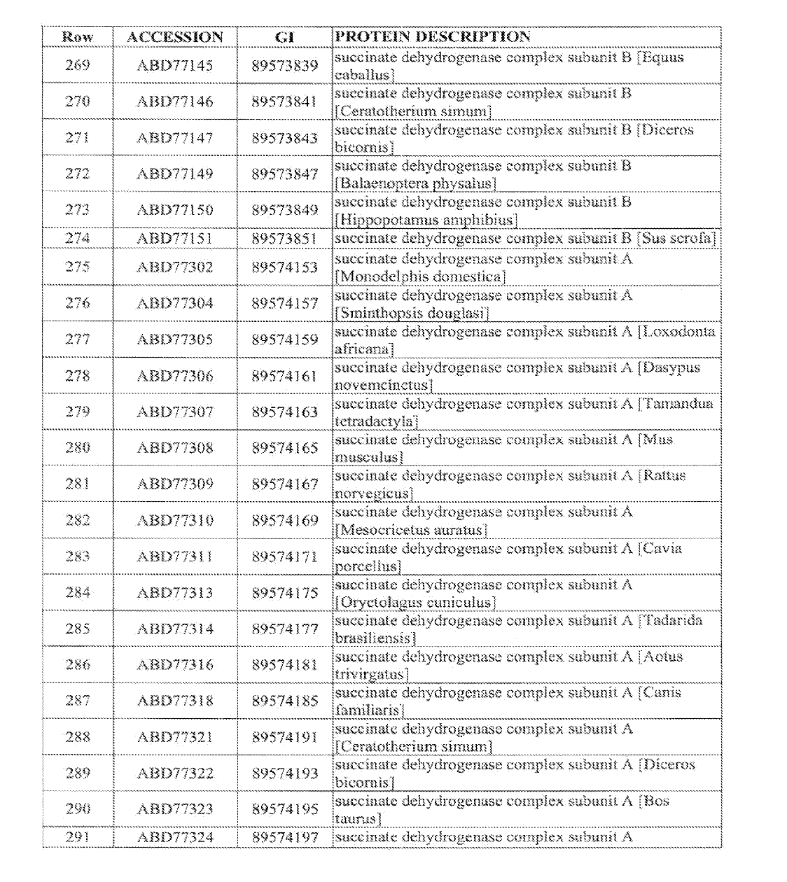

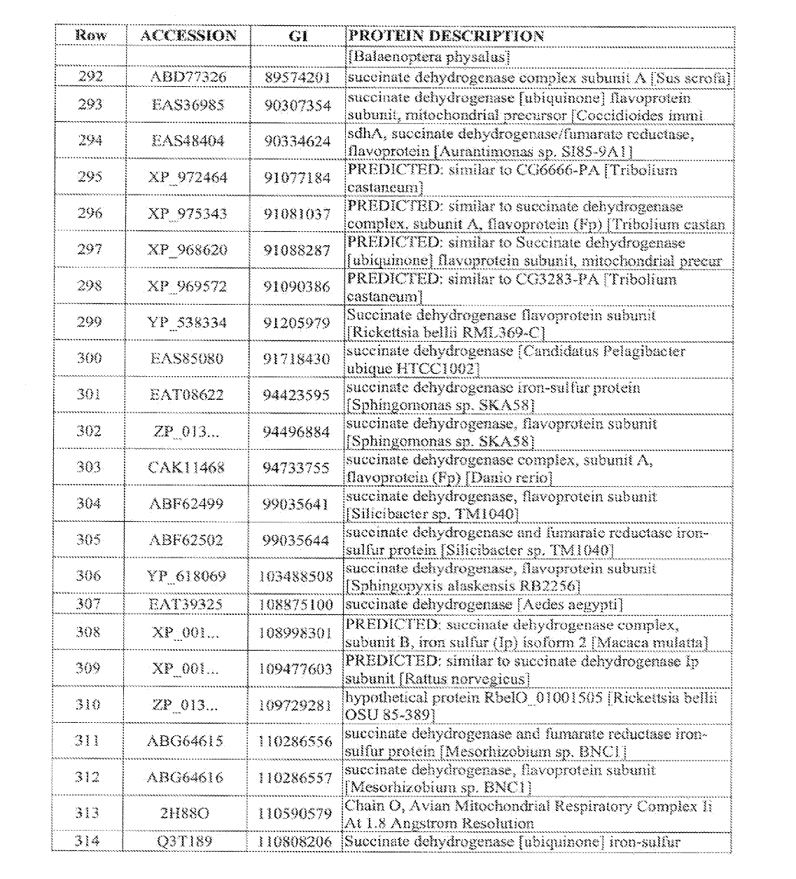

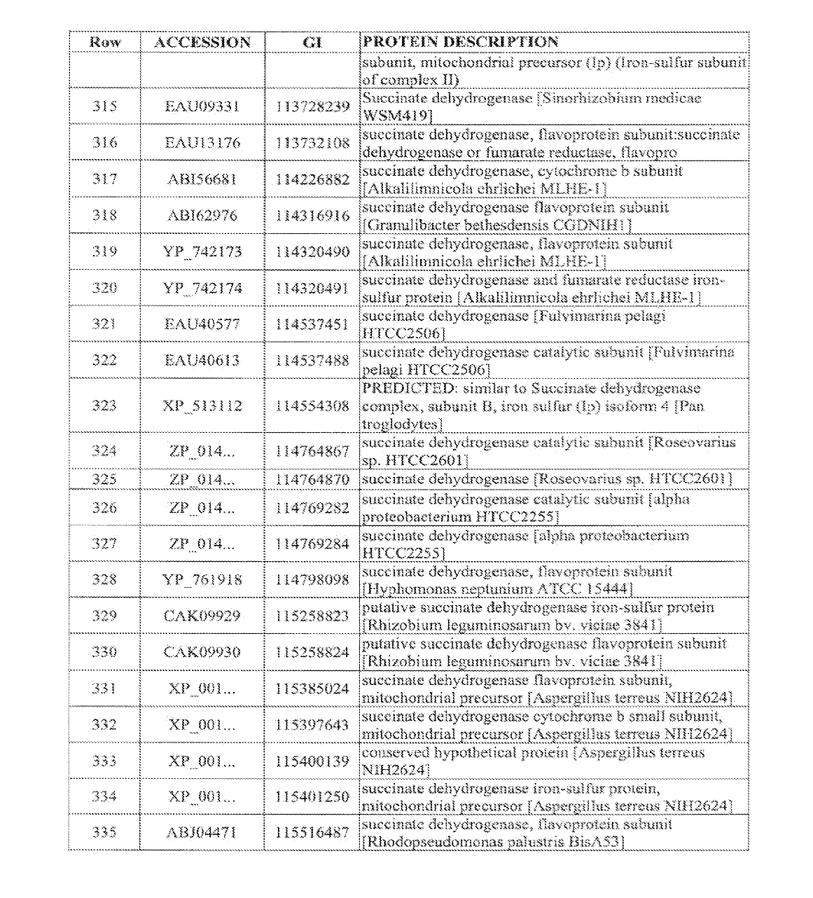

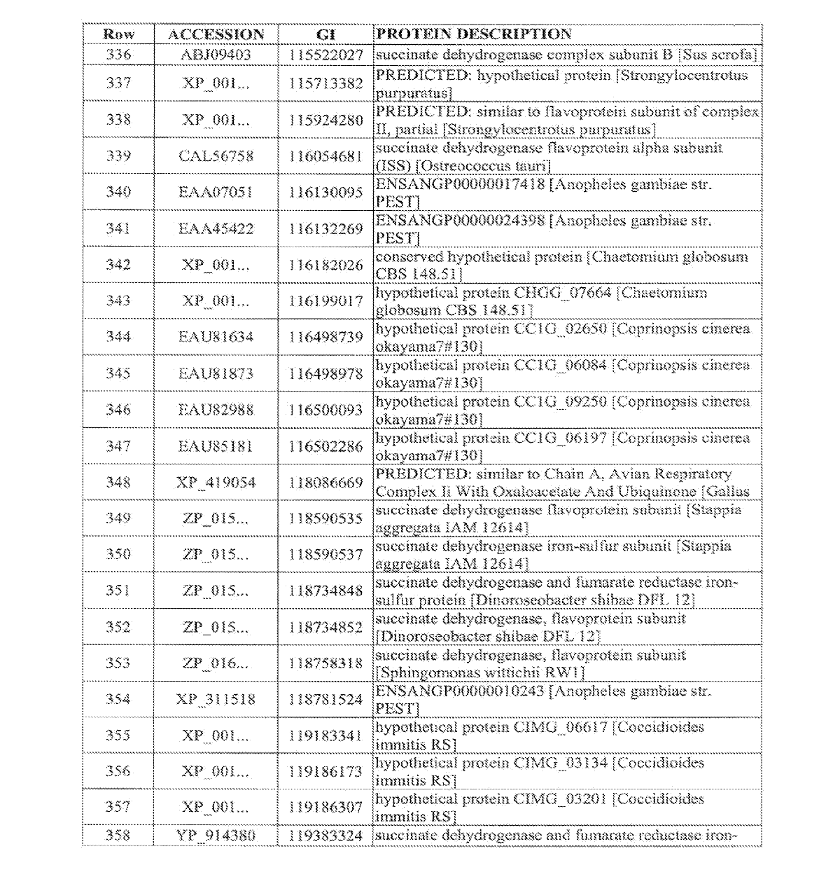

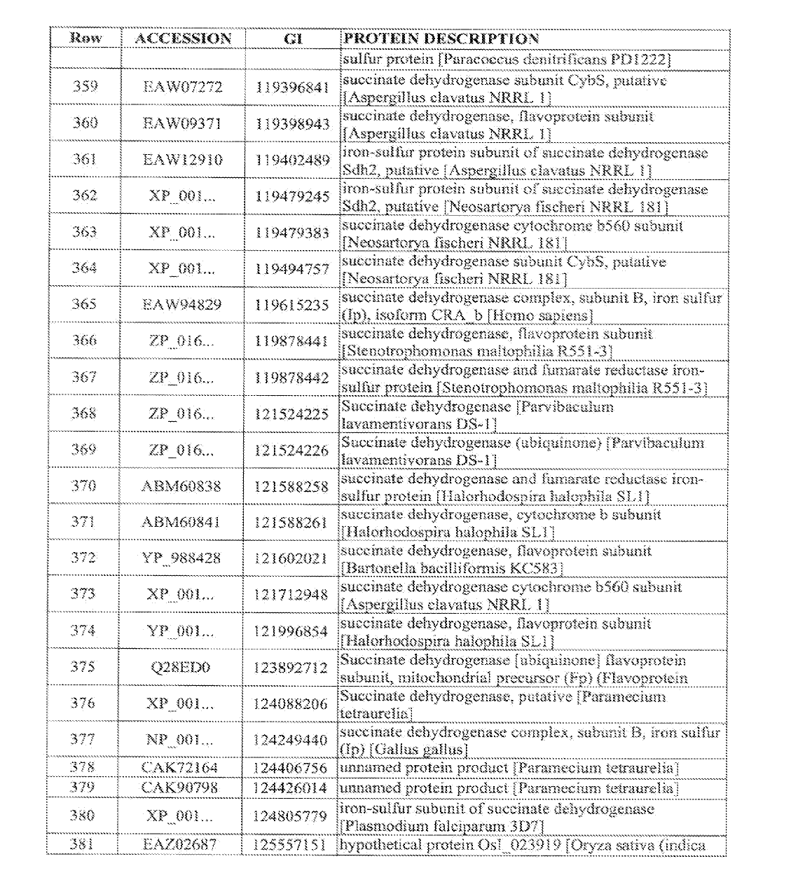

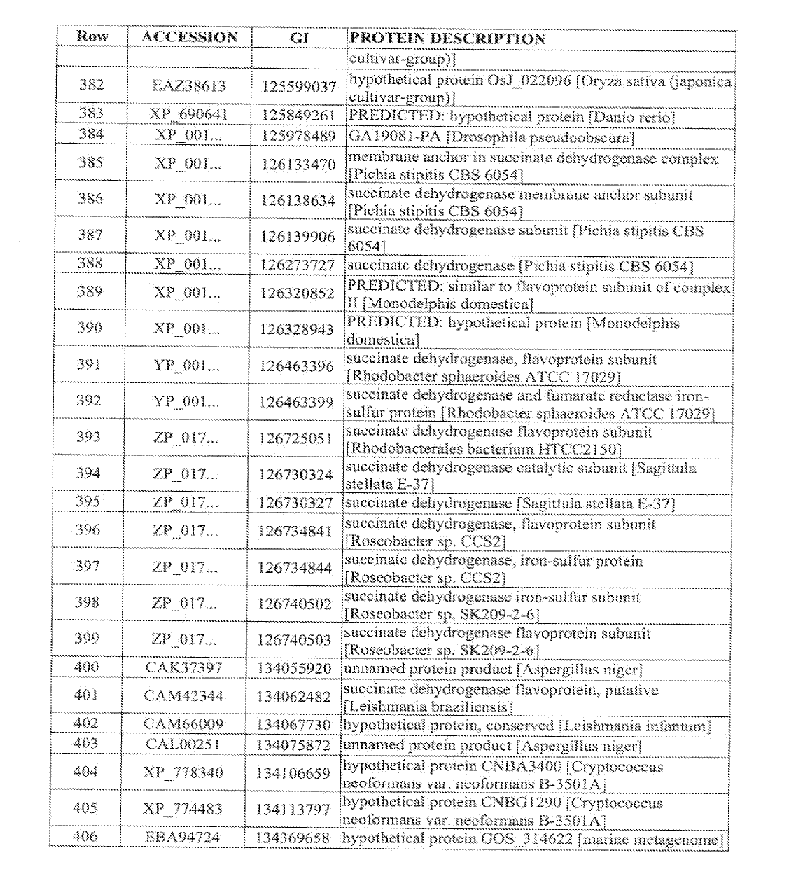

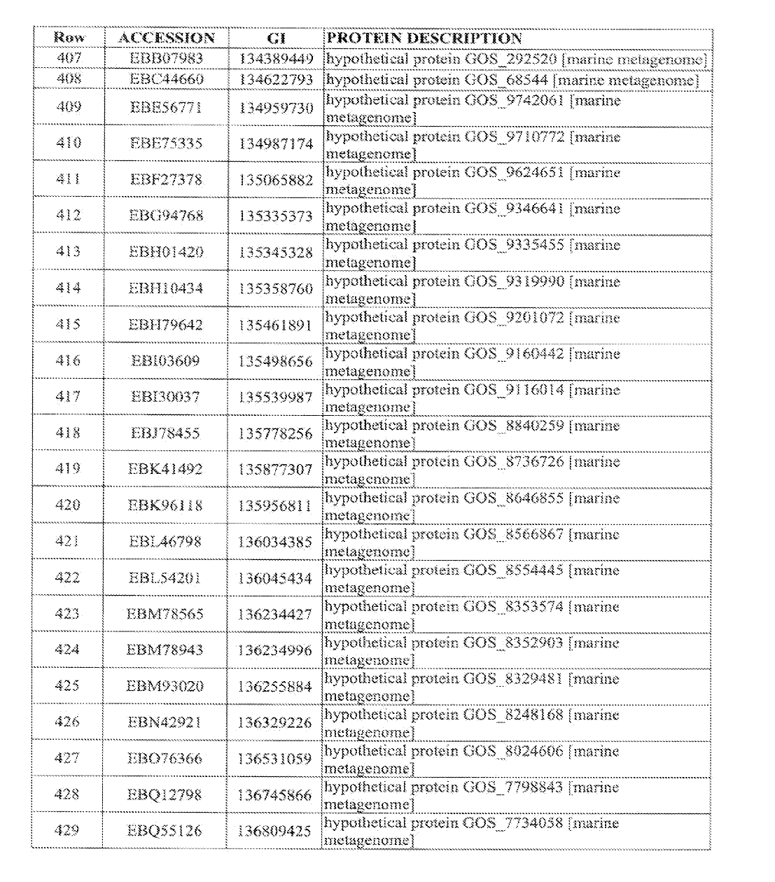

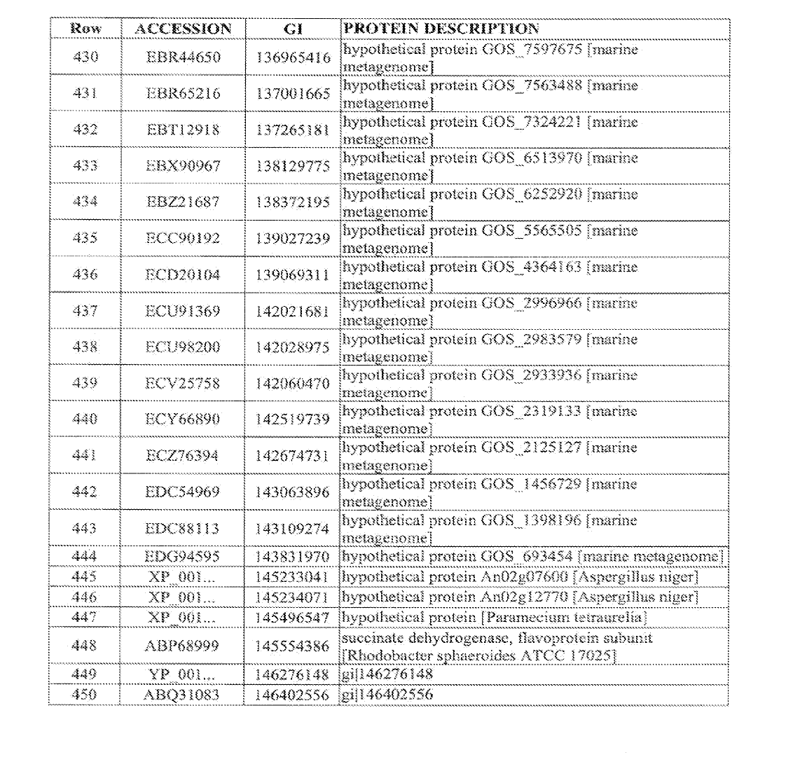

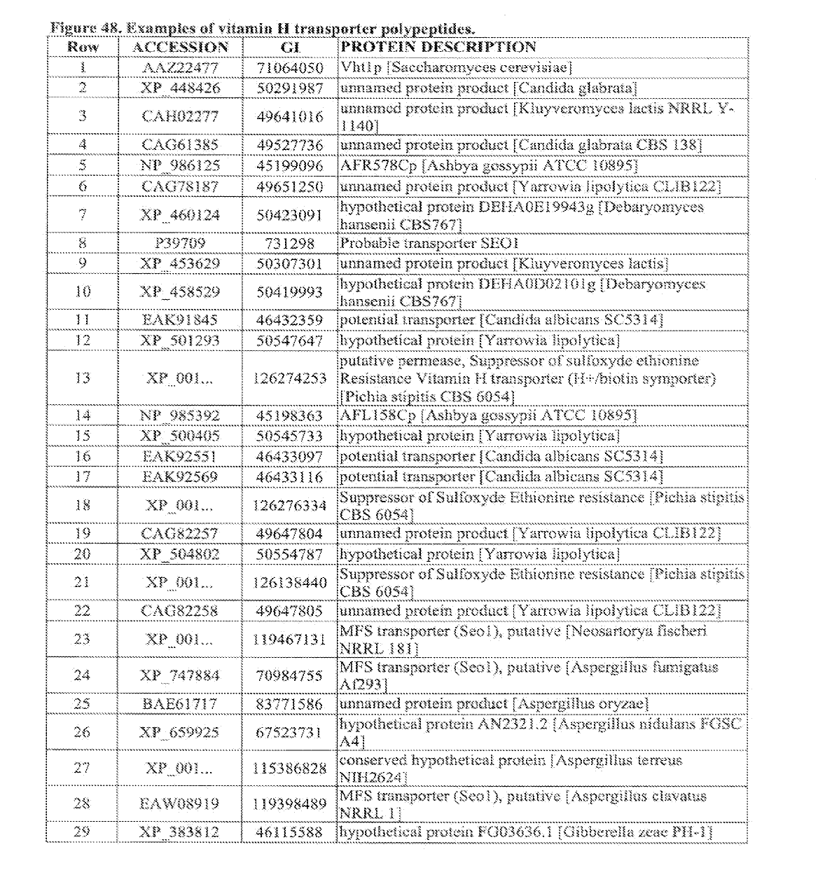

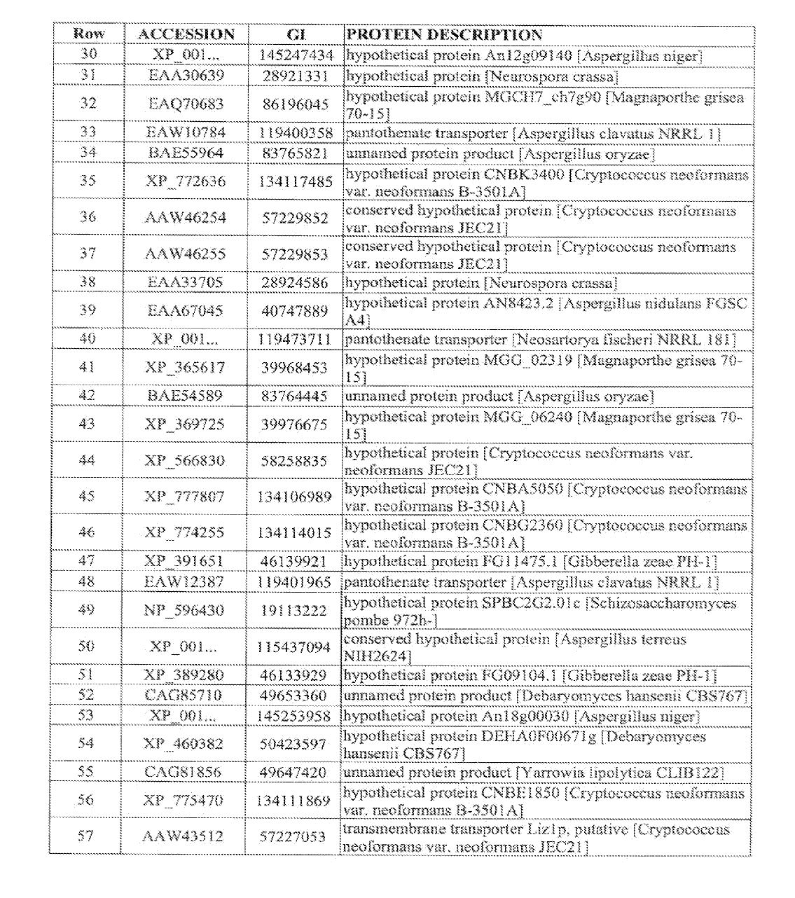

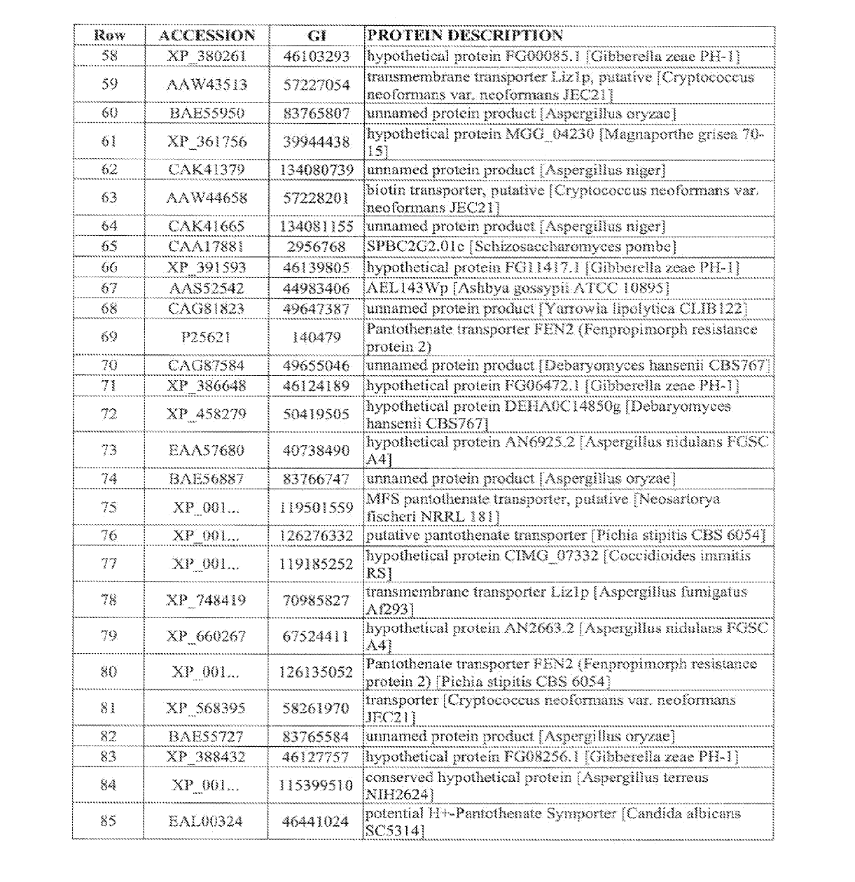

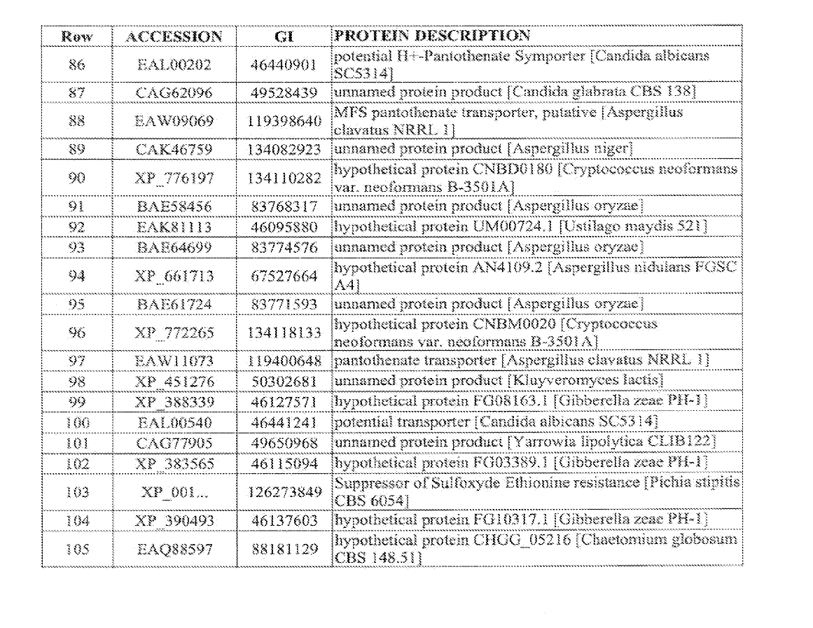

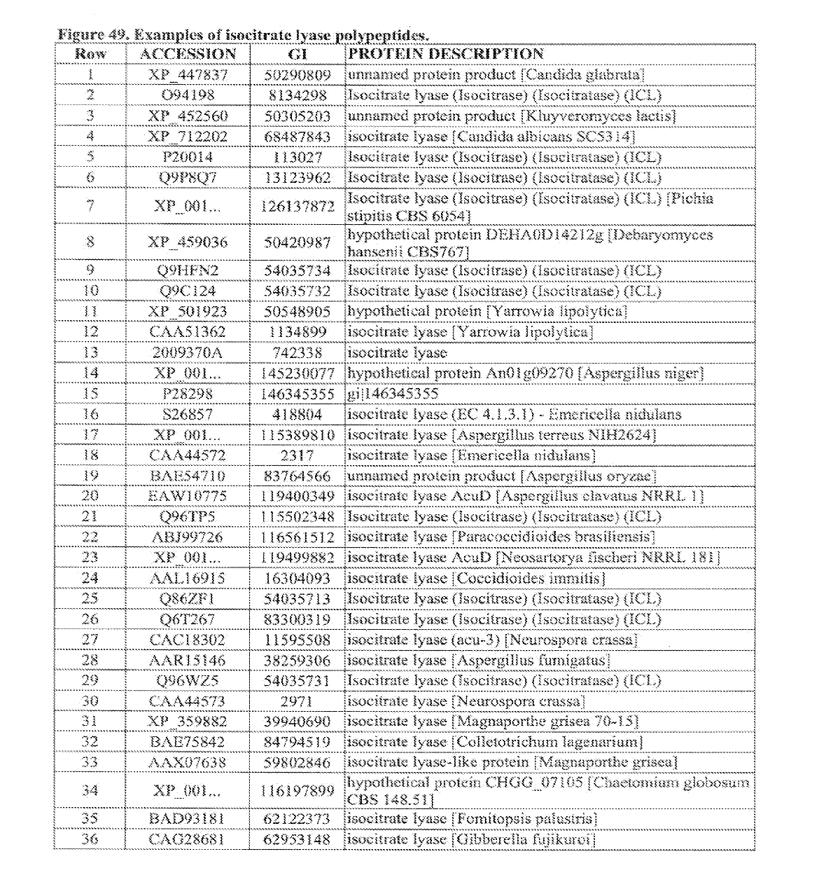

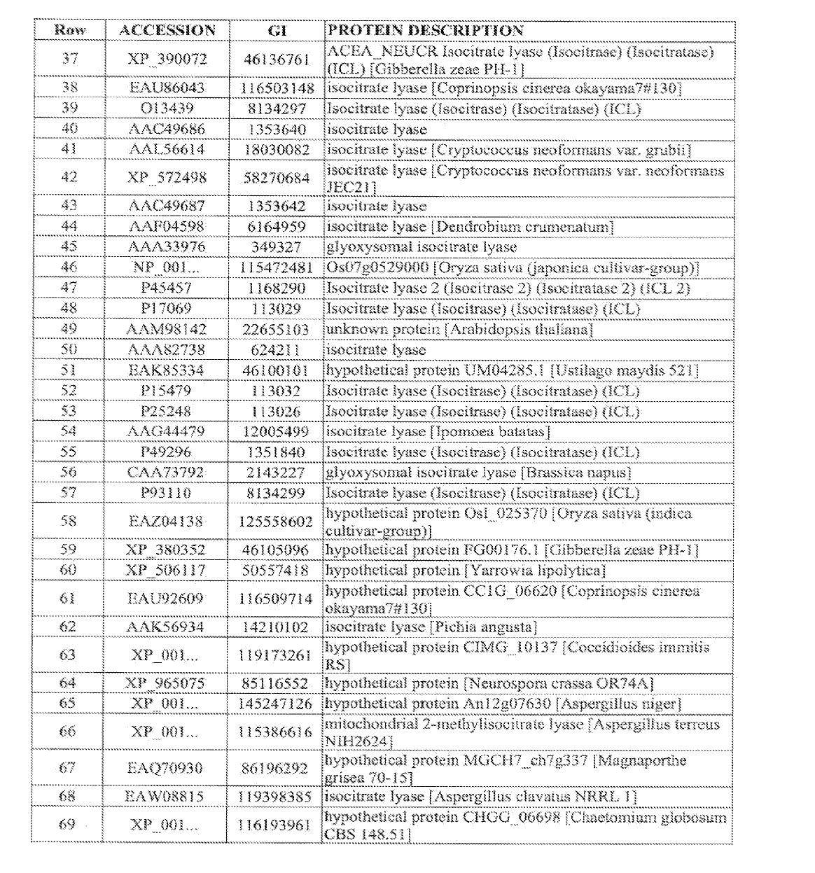

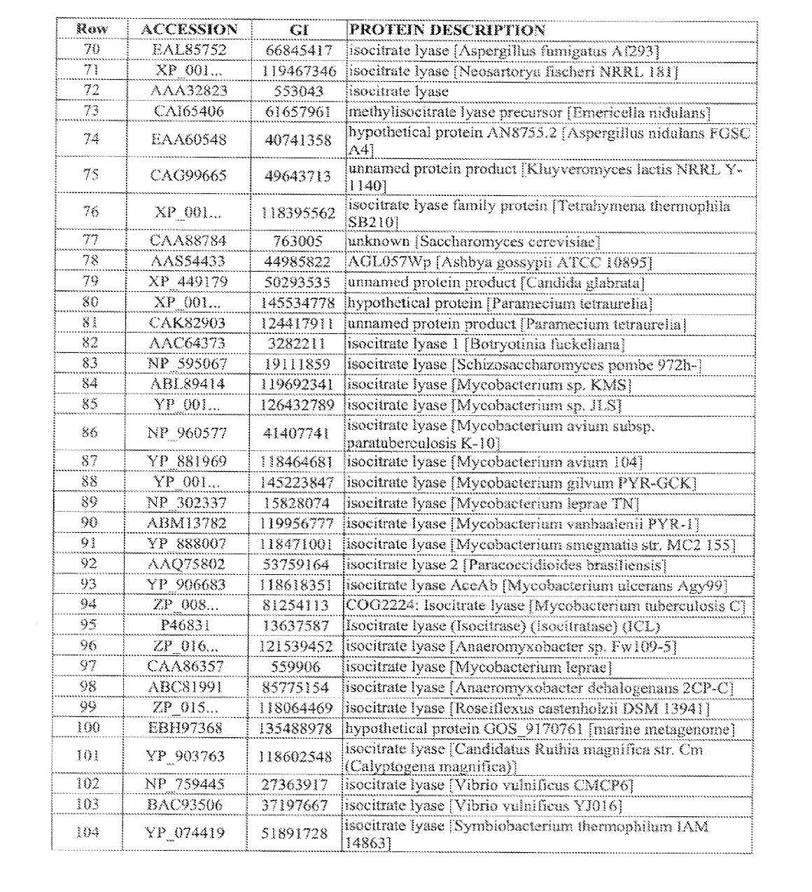

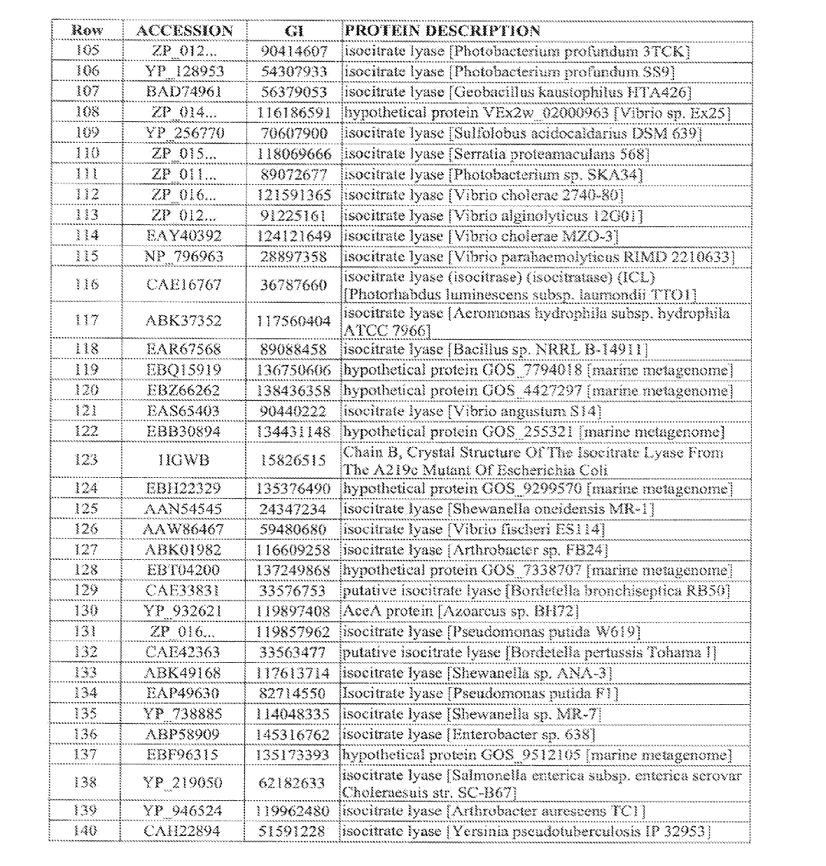

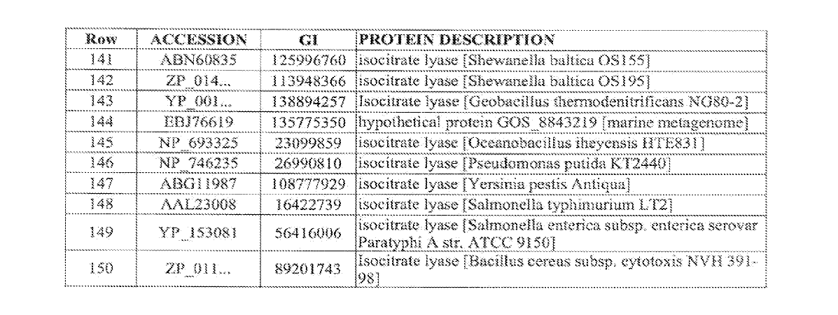

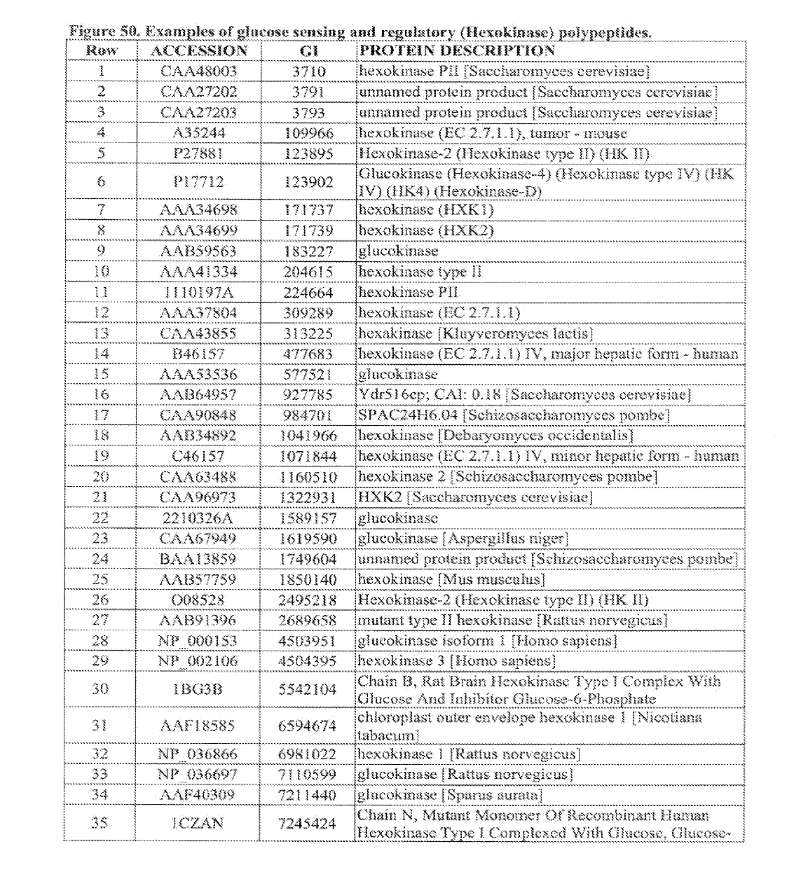

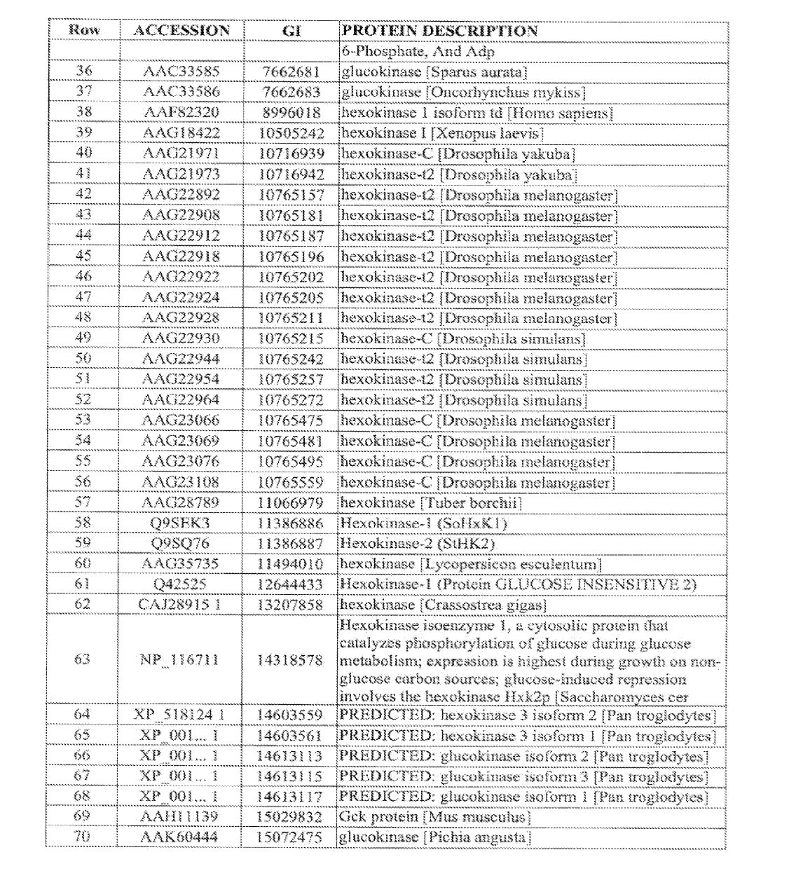

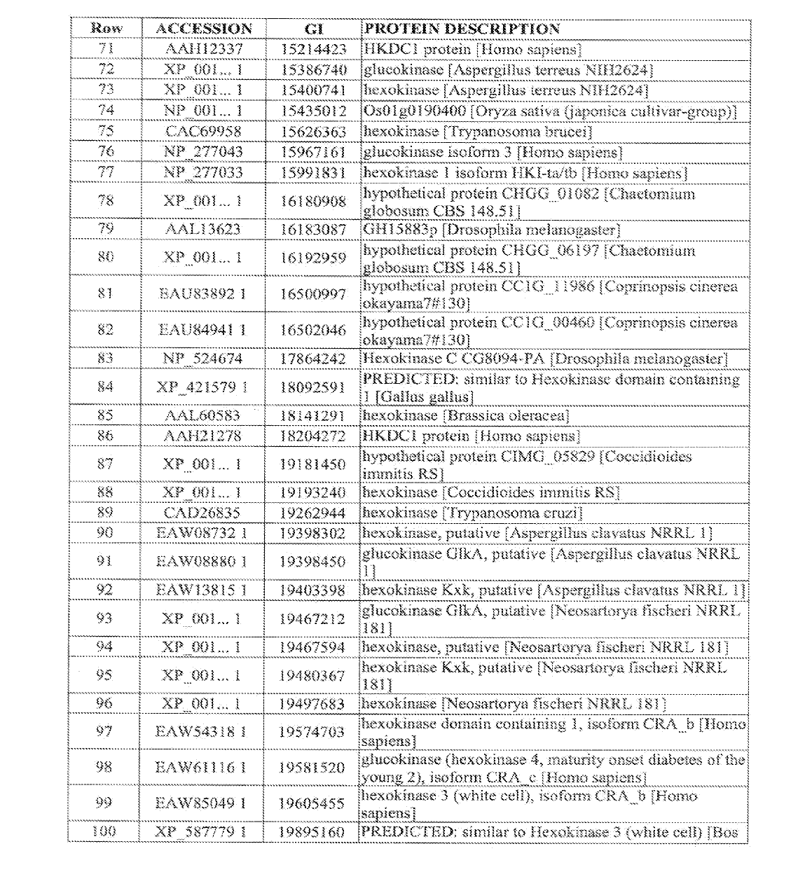

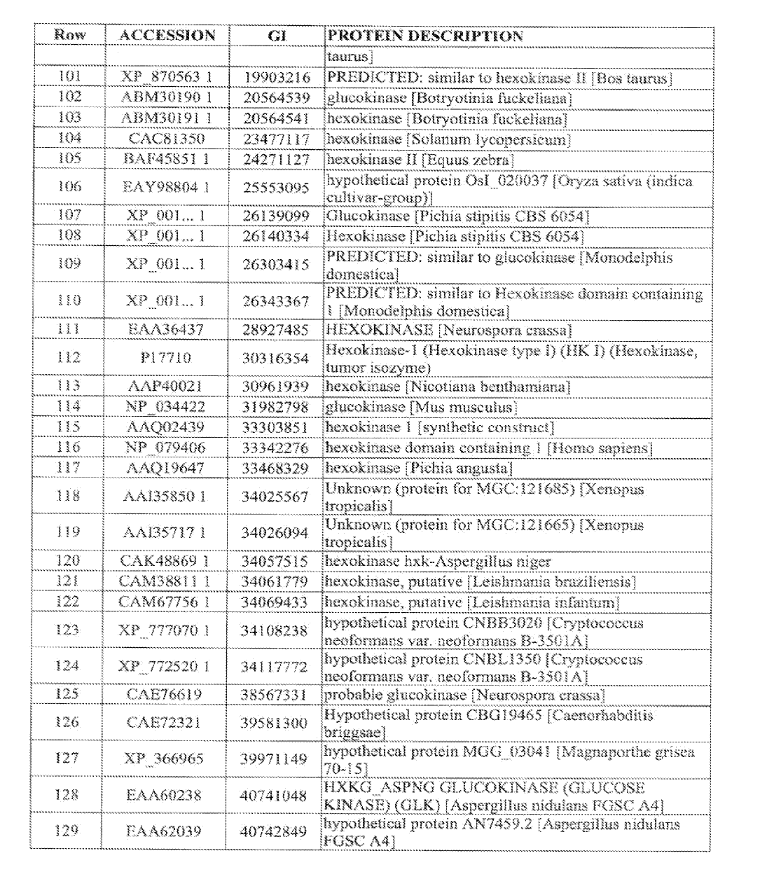

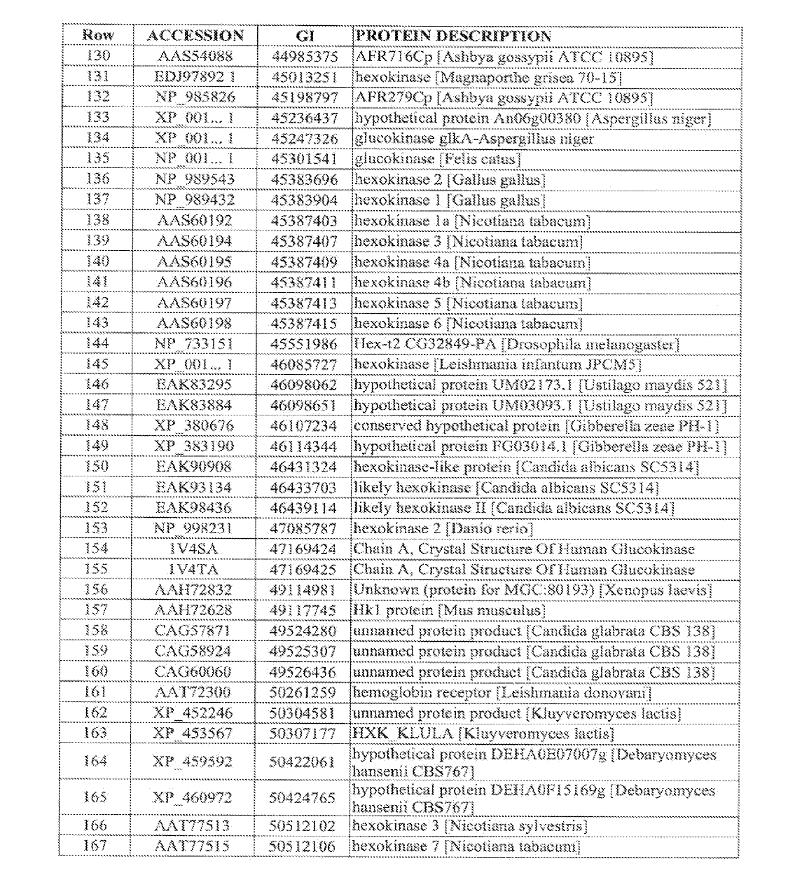

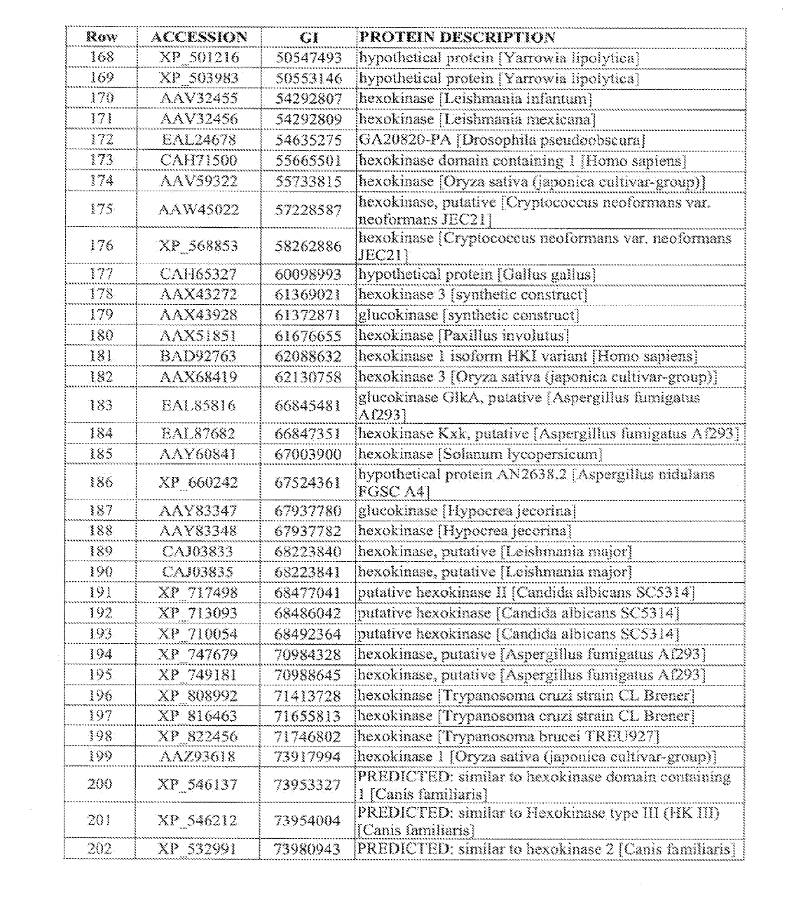

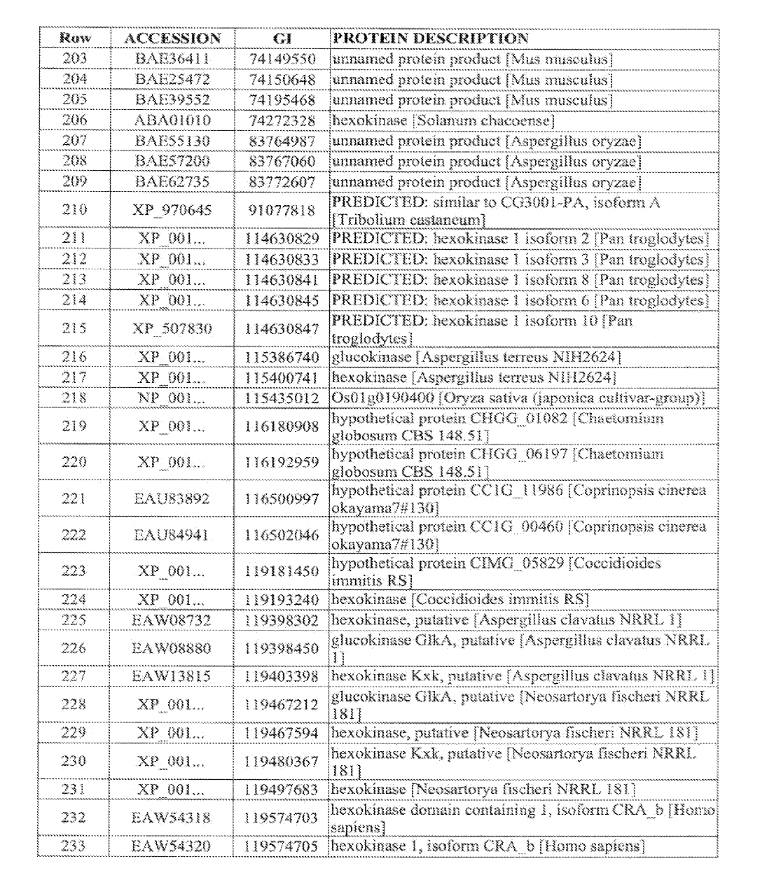

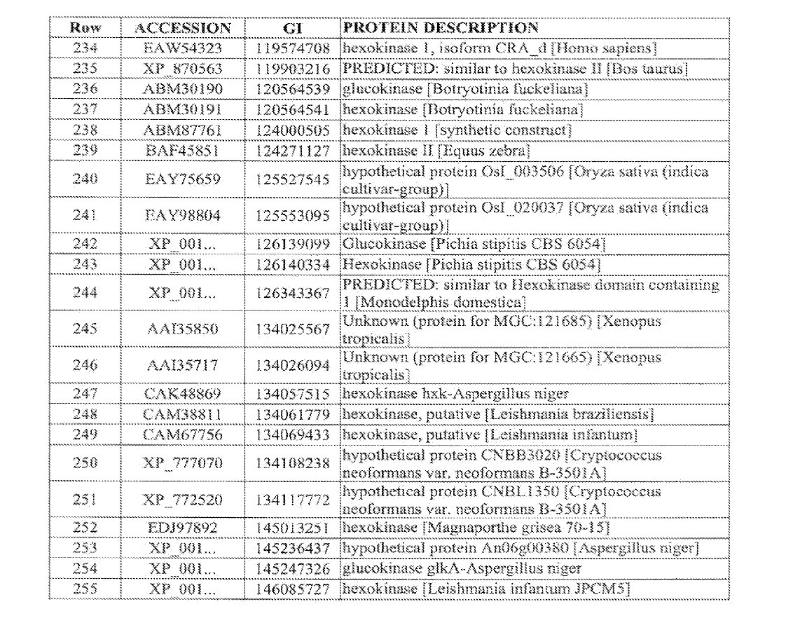

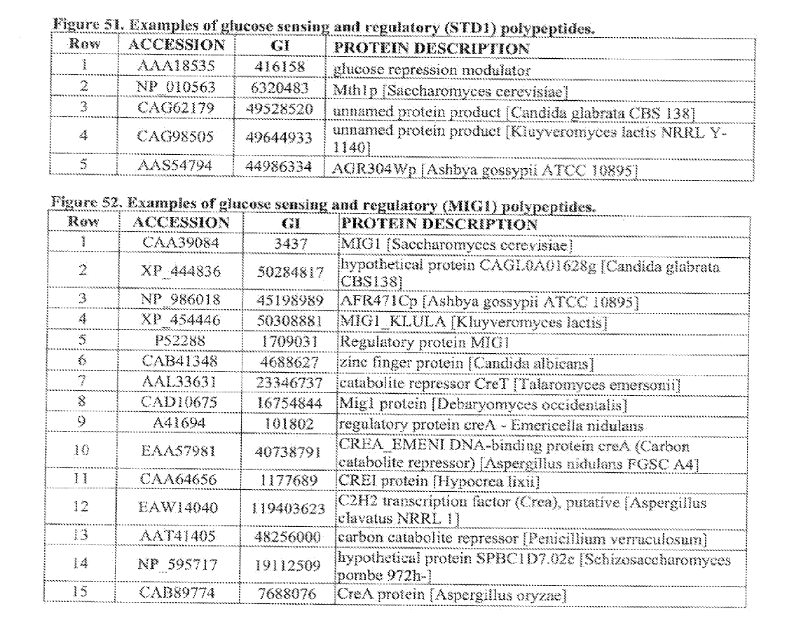

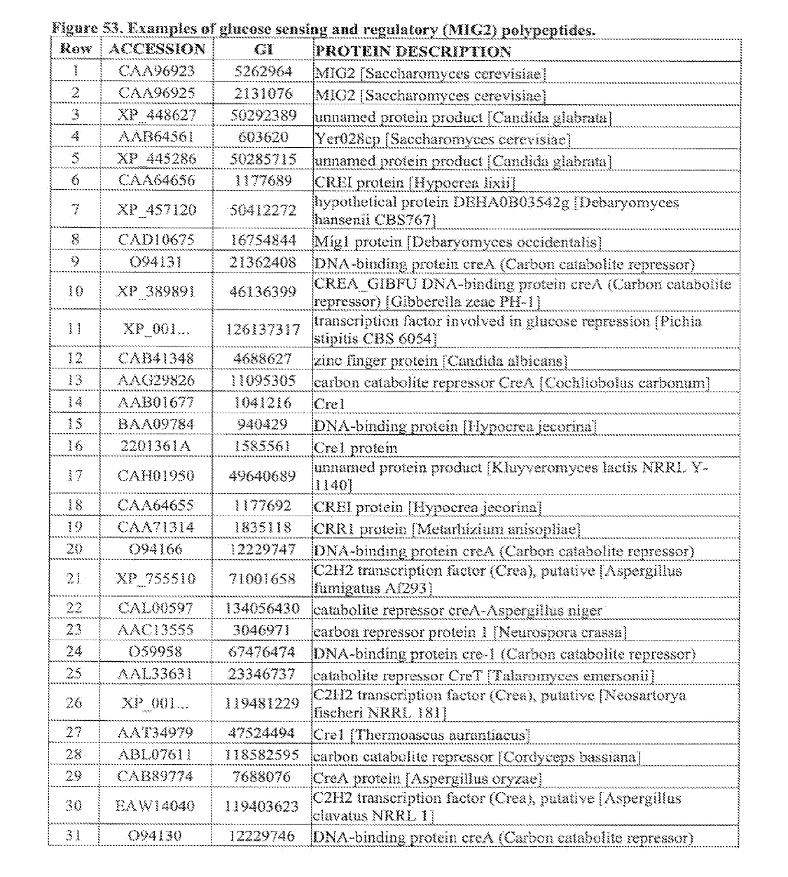

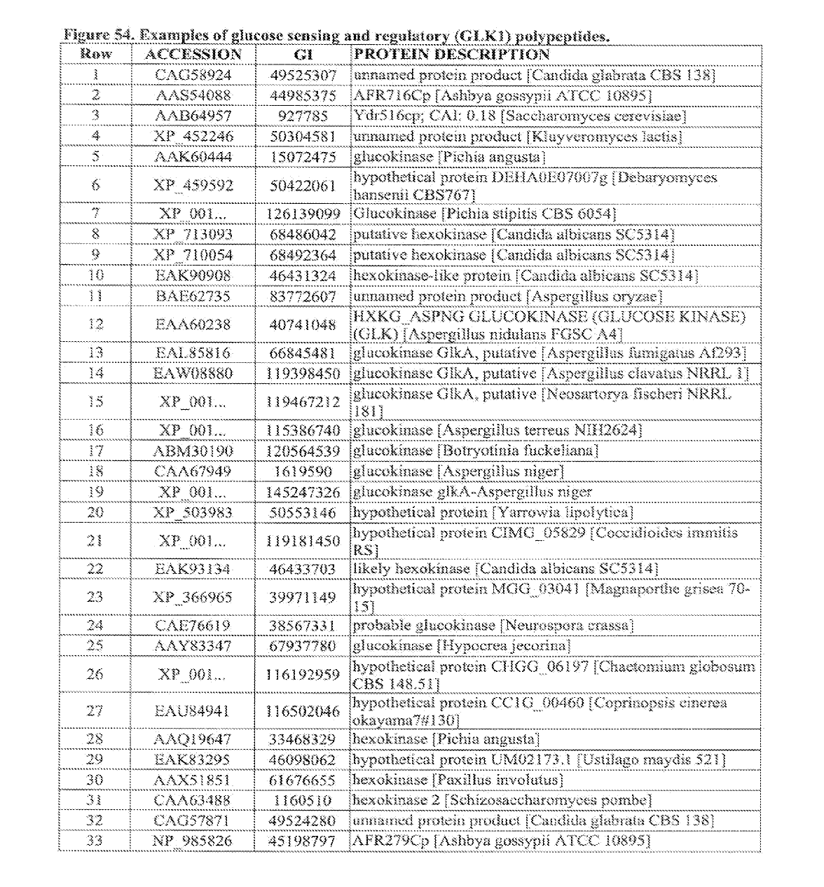

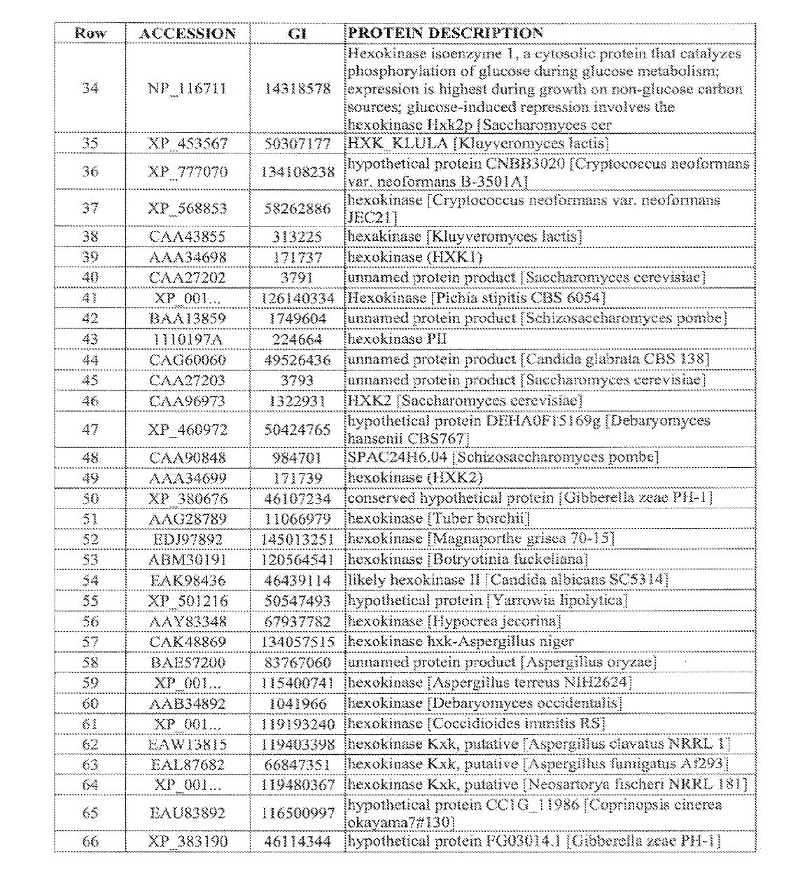

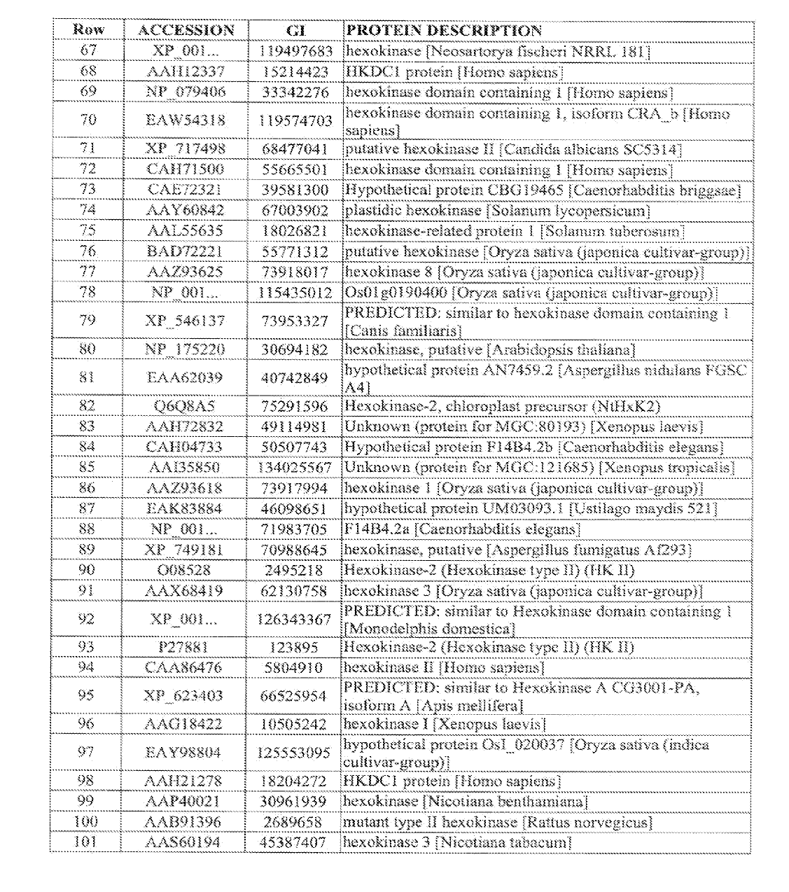

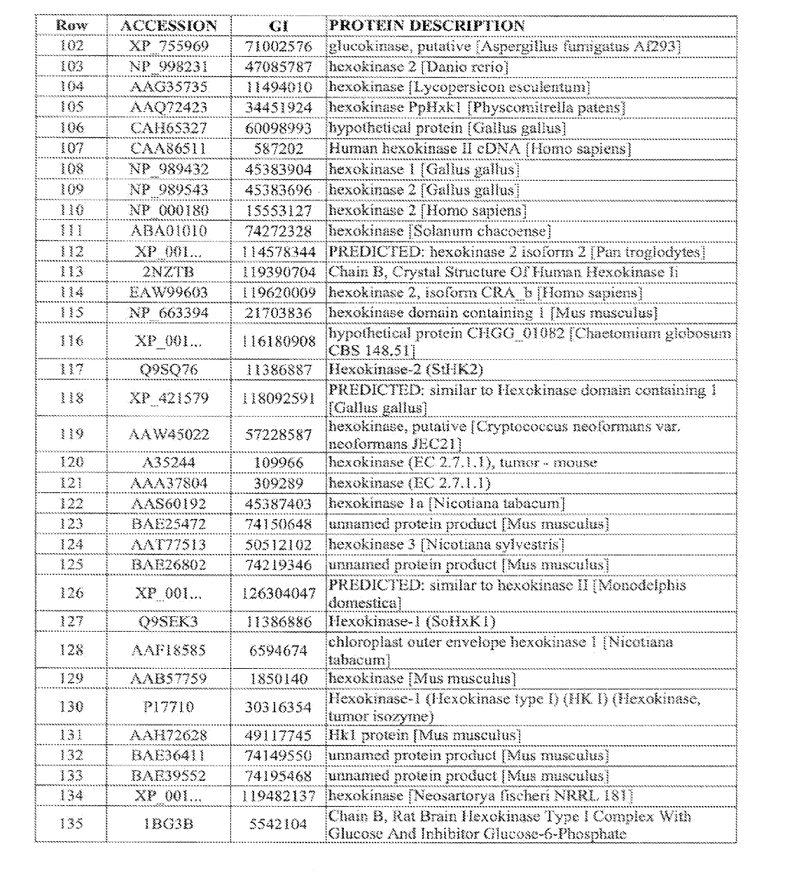

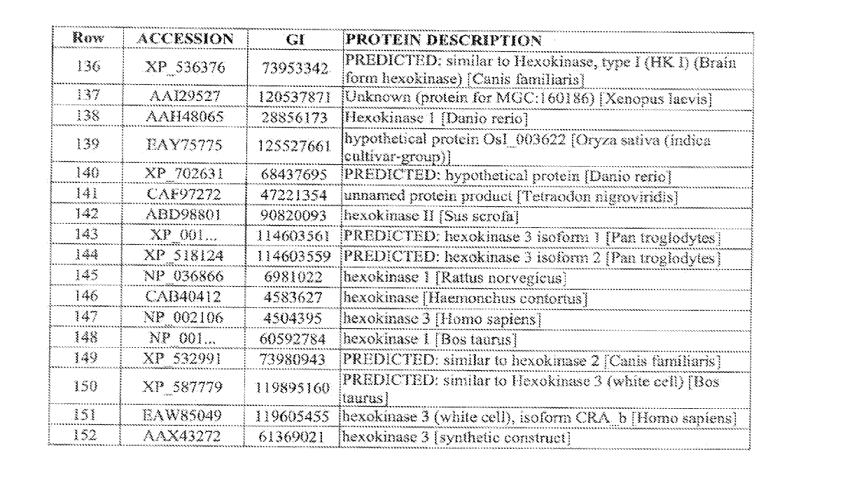

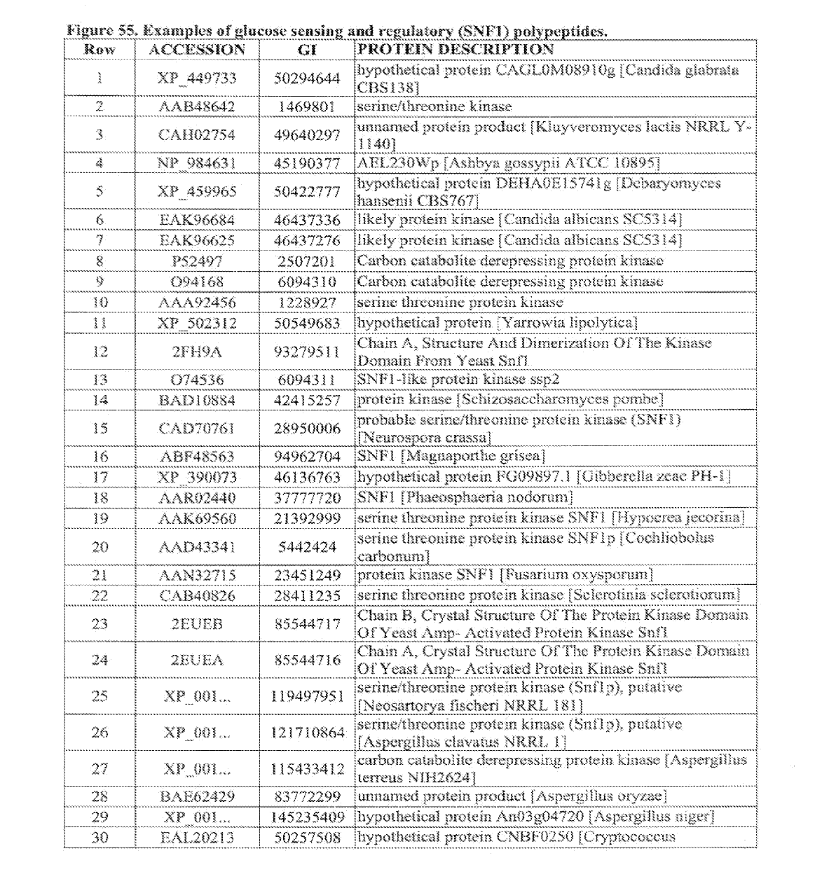

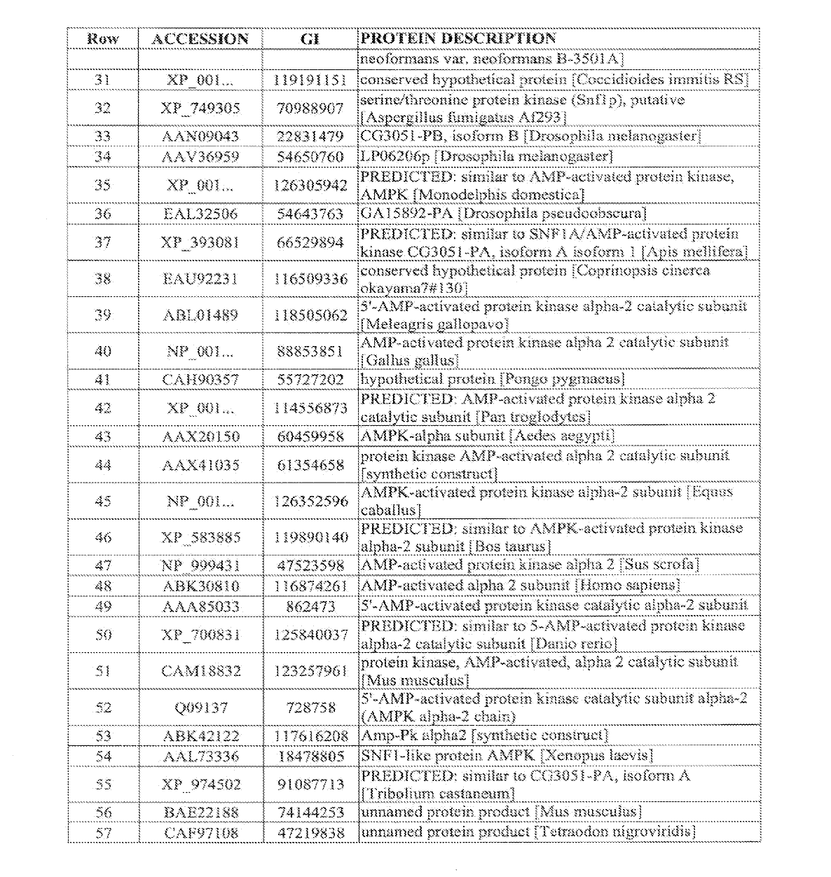

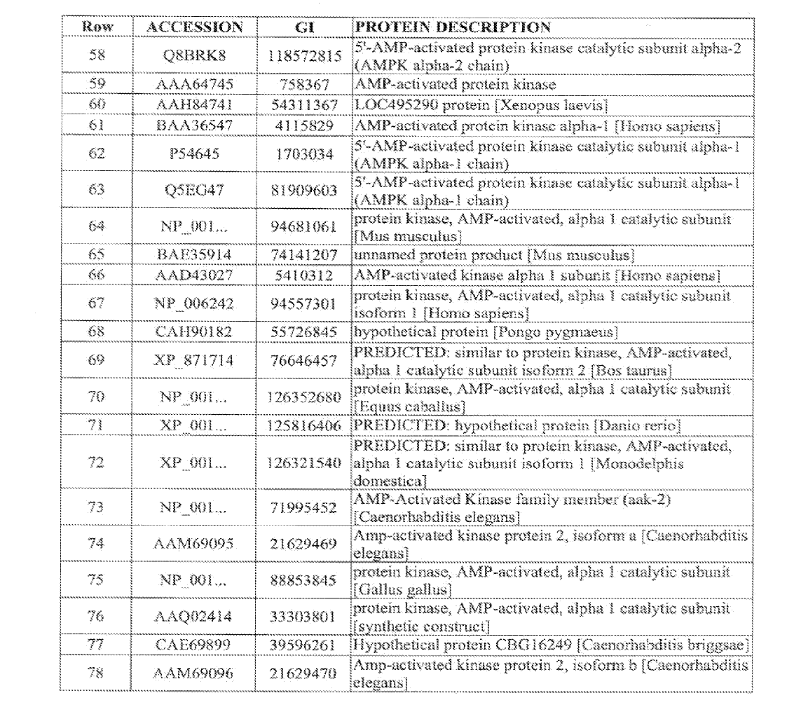

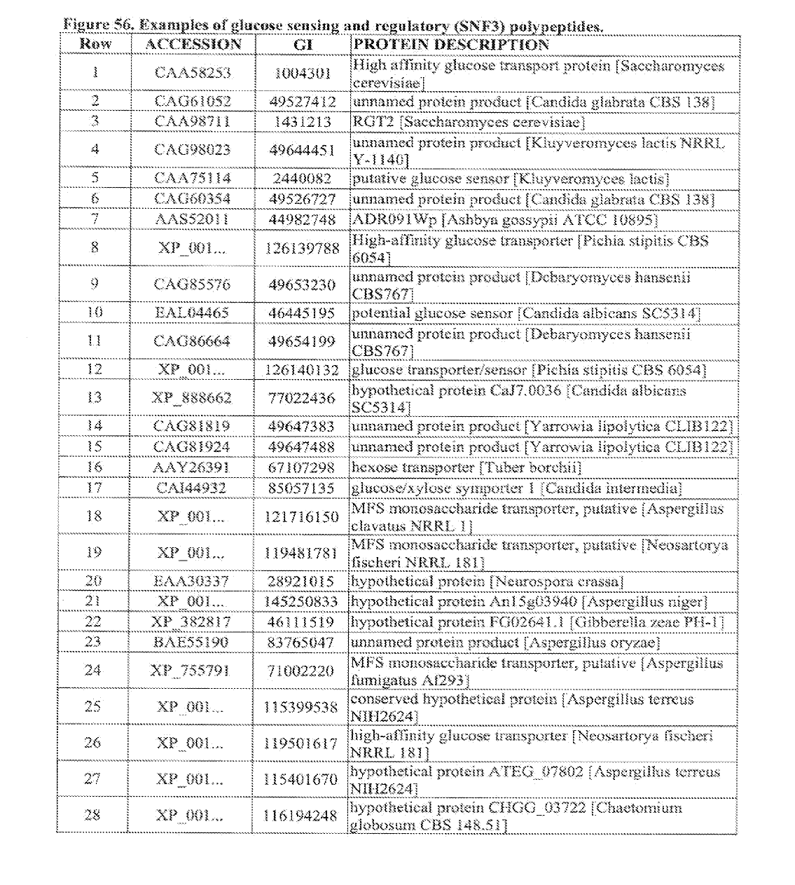

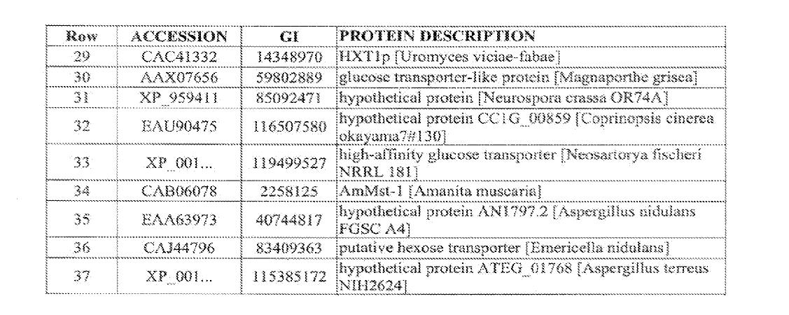

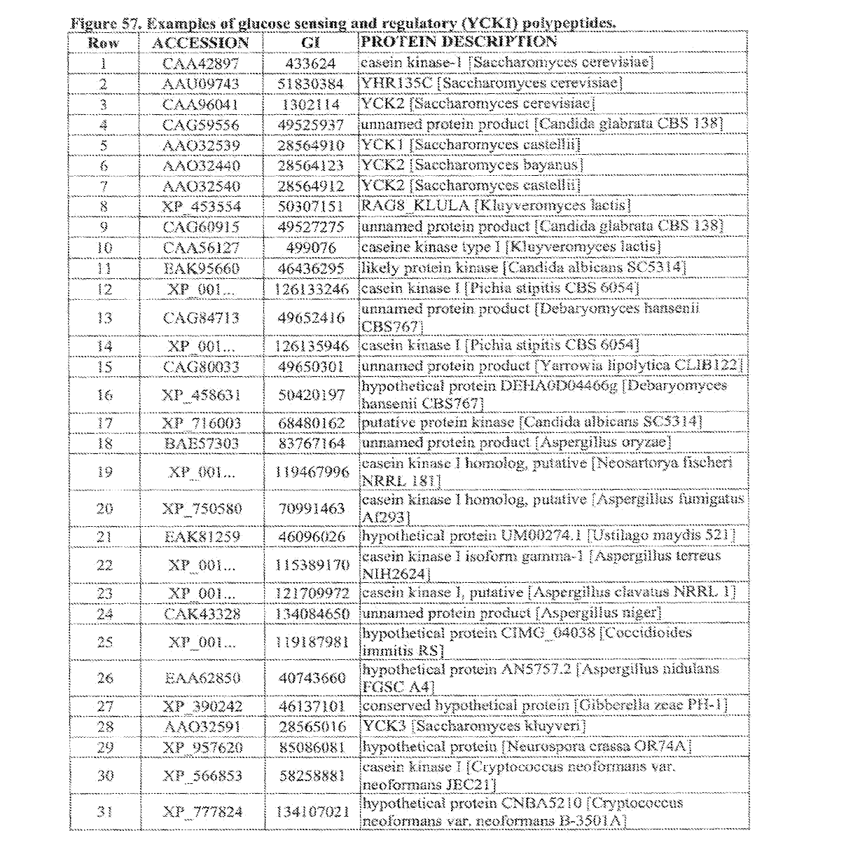

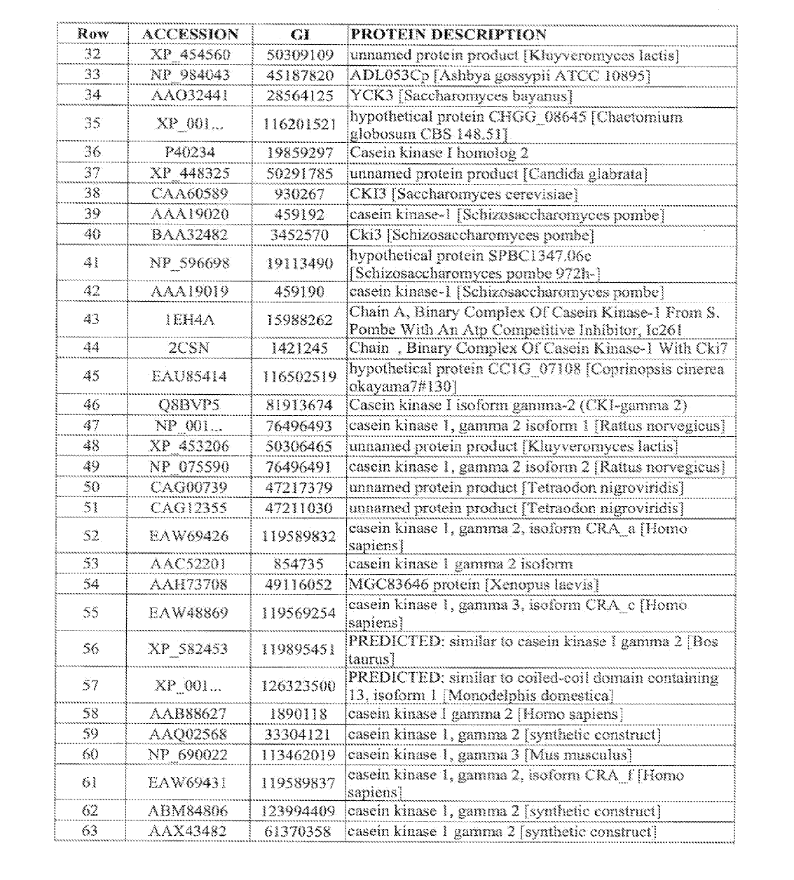

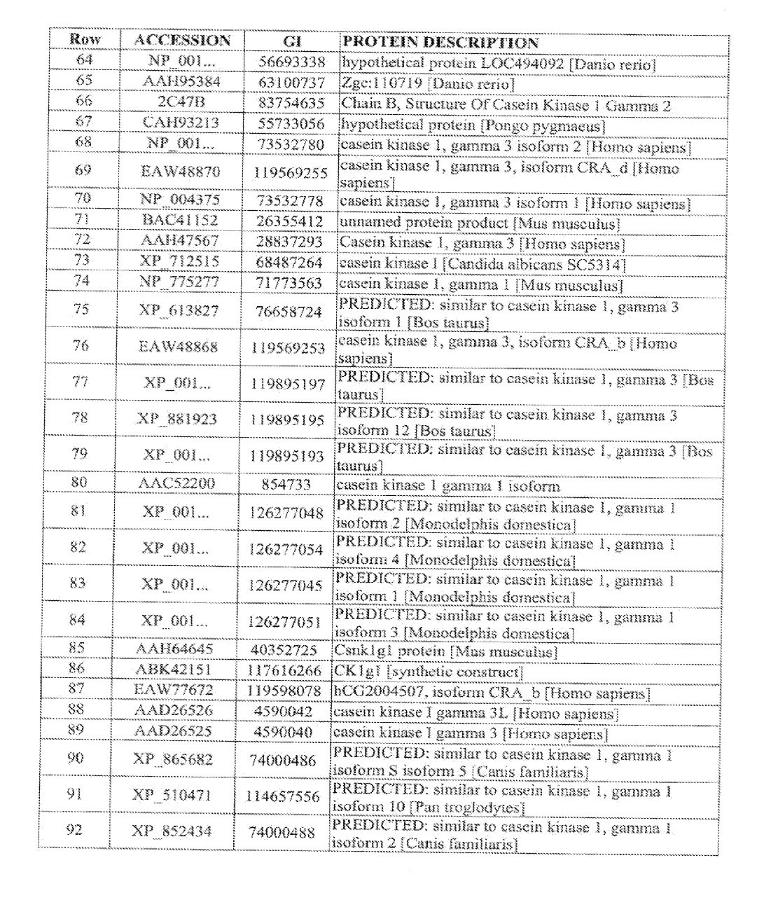

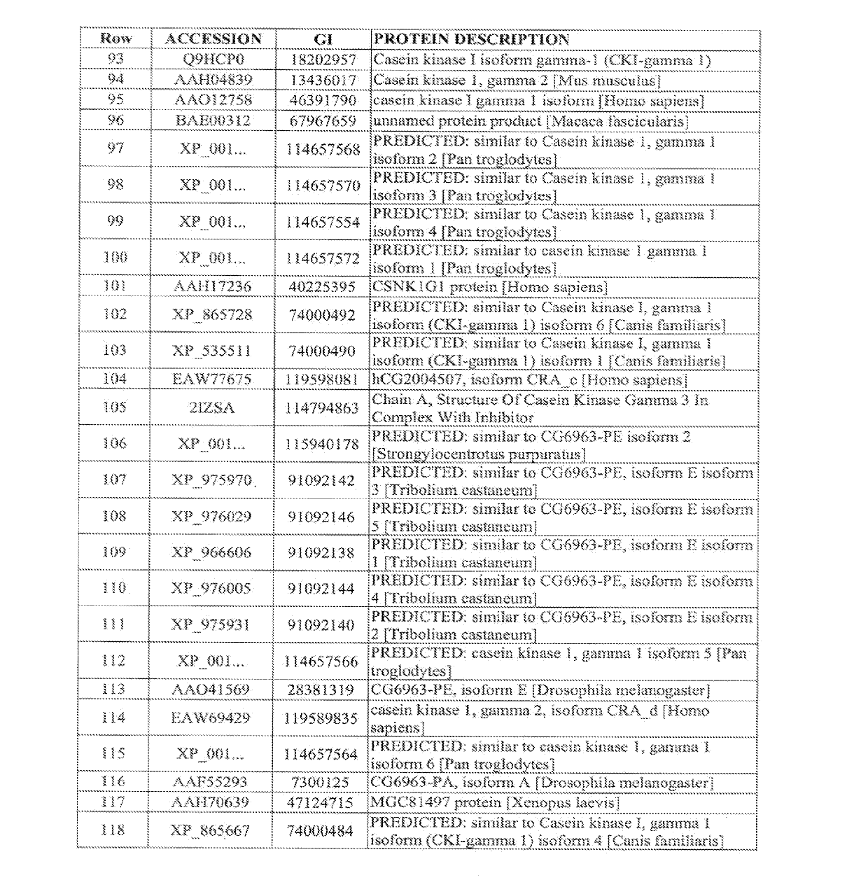

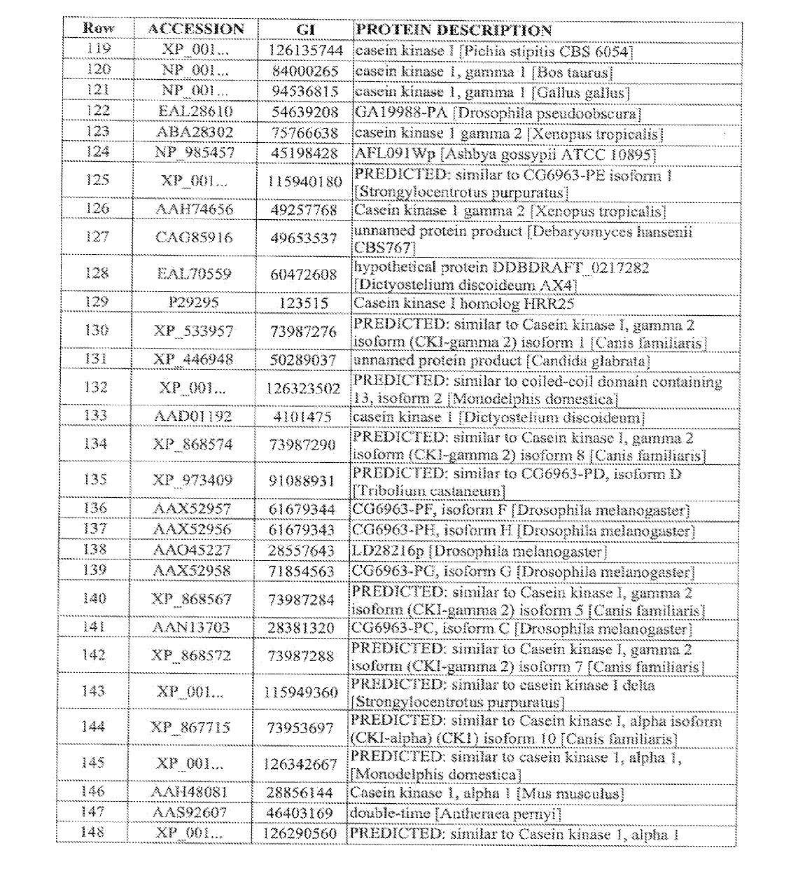

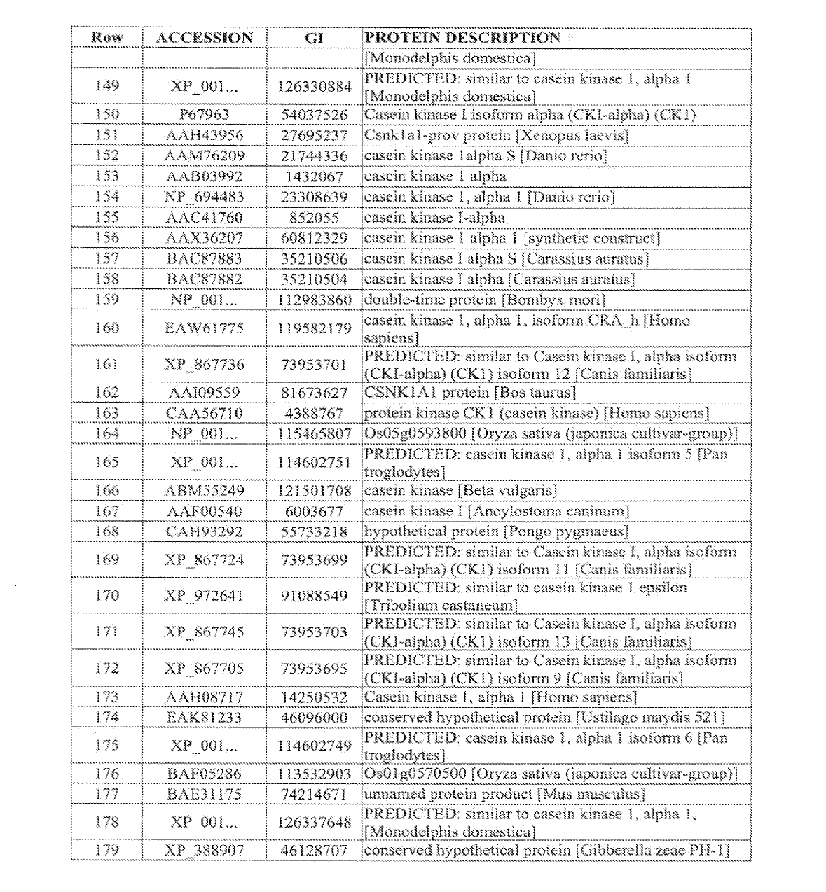

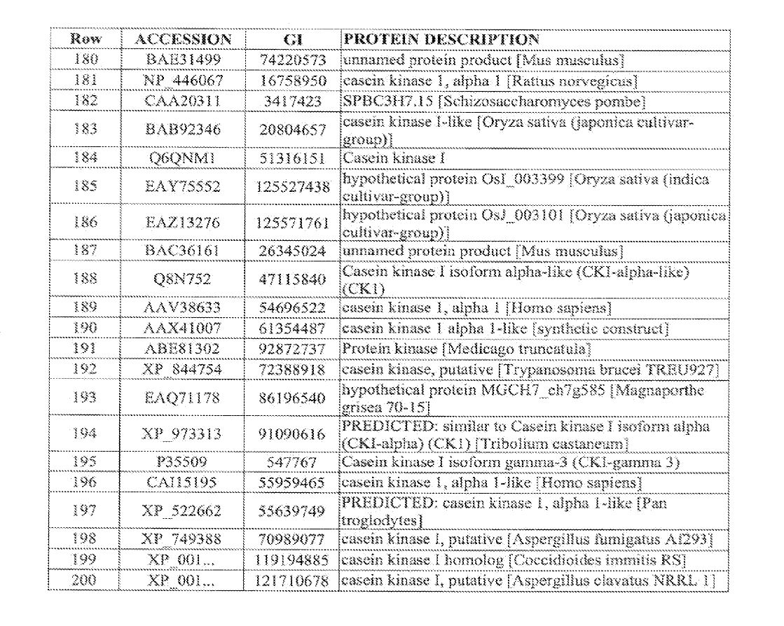

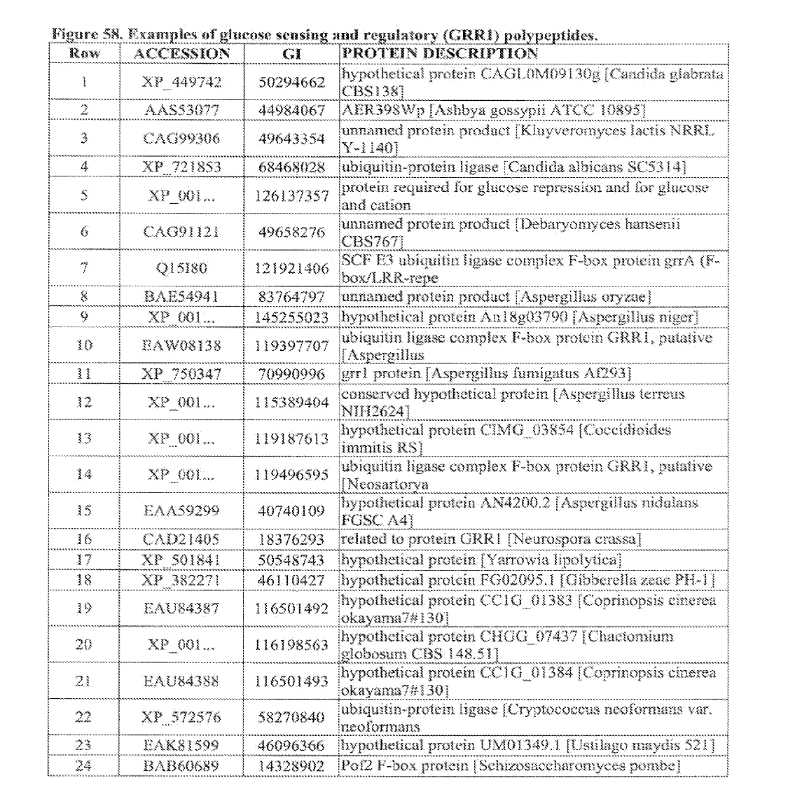

Glucose sensing and regulatory (GSR) polypeptides: “Glucose sensing and regulatory (GSR) polypeptides” are polypeptides that govern the complex physiological responses required for a fungal cell to utilize glucose efficiently and to the exclusion of other available carbon sources. GSR polypeptides include, among others, SNF1, MIG1, MIG2, HXK2, RGT1, SNF3, RGT2, STD1, MTH1, GRR1, YCK1, HXK1, and GLK1 polypeptides. Three regulatory systems appear to control most aspects of the glucose sensing response. S. cerevisiae and other fungi naturally produce GSR polypeptides. For example, the S. cerevisiae SNF1/MIG1 system functions to repress (high glucose) or derepress (low glucose) expression of a broad set of genes involved in the utilization of alternative carbon sources and in gluconeogenesis. In response to glucose depletion, phosphorylation of the MIG1 transcriptional repressor by the SNF1 kinase prevents both nuclear localization of the repressor and its binding to recognition sequences. MIG2, which binds to a recognition site similar to that of MIG1, and HXK2 are additional proteins implicated in controlling the expression of this set of genes. A second regulatory system, which functions primarily to regulate expression of hexose transporter (HXT) polypeptides, impinges on the action of the RGT1 transcriptional repressor. In brief, glucose sensing proteins (SNF3 and RGT2) that are homologues of glucose transporters initiate a signal that is relayed to the paralogous MTH1 and STD1 proteins, which are necessary for RGT1-mediated repression. When glucose binds sensors, the MTH1 and STD1 proteins are phosphorylated by the YCK1 kinase, and this phosphorylation targets the MTH1 and STD1 proteins for GRR1 mediated ubiquitination and degradation. Significant cross-talk is also exhibited between these first two glucose sensing systems. For example, MTH1 gene expression is controlled by the MIG1 and MIG2 repressor proteins. A third glucose sensing system, which requires proteins such as, but not limited to, the GPR1 G-protein coupled receptor and hexokinases (e.g. HXK1, HXK2, and GLK1), regulates transcriptional and other cellular responses that result from glucose-mediated activation of cAMP synthesis. Examples of glucose sensing and regulatory (GSR) polypeptides are represented by the Genbank GI numbers in FIGS. 50-61 and the SNF1, MIG1, MIG2, HXK2, RGT1, SNF3, RGT2, STD1, MTH1, GRR1, YCK1, HXK1, and GLK1 polypeptides in FIG. 29. In some cases, a glucose sensing and regulatory (GSR) polypeptide has an amino acid sequence that is at least about 75%, 85%, 90%, 95% or 100% identical to that of a polypeptide represented by the Genbank GI numbers in FIGS. 50-61 or a SNF1, MIG1, MIG2, HXK2, RGT1, SNF3, RGT2, STD1, MTH1, GRR1, YCK1, HXK1, or GLK1 polypeptide in FIG. 29.

Heterologous: The term “heterologous”, means from a source organism other than the host cell. For example, “heterologous” as used herein refers to genetic material or polypeptide that does not naturally occur in the species in which it is present and/or being expressed. It will be understood that, in general, when heterologous genetic material or polypeptide is selected for introduction into and/or expression by a host cell, the particular source organism from which the heterologous genetic material or polypeptide may be selected is not critical to the practice of the present invention. Relevant considerations may include, for example, how closely related the potential source and host organisms are in evolution, or how related the source organism is with other source organisms from which sequences of other relevant polypeptides have been selected. Where a plurality of different heterologous polypeptides and/or nucleic acids are to be introduced into and/or expressed by a host cell, different polypeptides or nucleic acids may be from different source organisms, or from the same source organism. To give but one example, in some cases, individual polypeptides may represent individual subunits of a complex protein activity and/or may be required to work in concert with other polypeptides in order to achieve the goals of the present invention. In some embodiments, it will often be desirable for such polypeptides to be from the same source organism, and/or to be sufficiently related to function appropriately when expressed together in a host cell. In some embodiments, such polypeptides may be from different, even unrelated source organisms. It will further be understood that, where a heterologous polypeptide is to be expressed in a host cell, it will often be desirable to utilize nucleic acids whose sequences encode the polypeptide that have been adjusted to accommodate codon preferences of the host cell and/or to link the encoding sequences with regulatory elements active in the host cell.

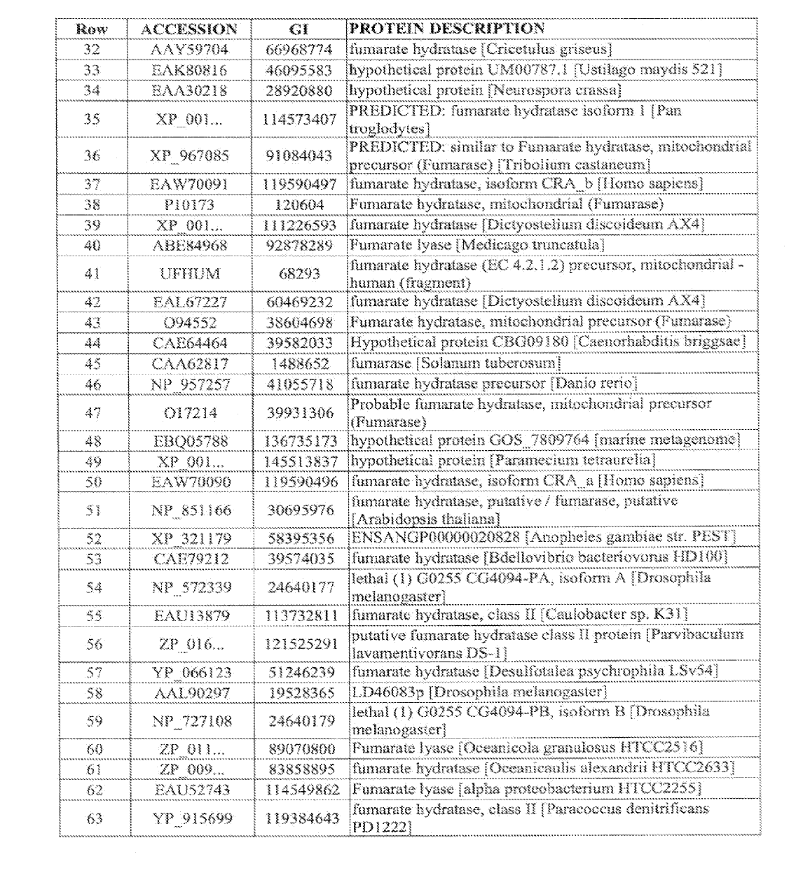

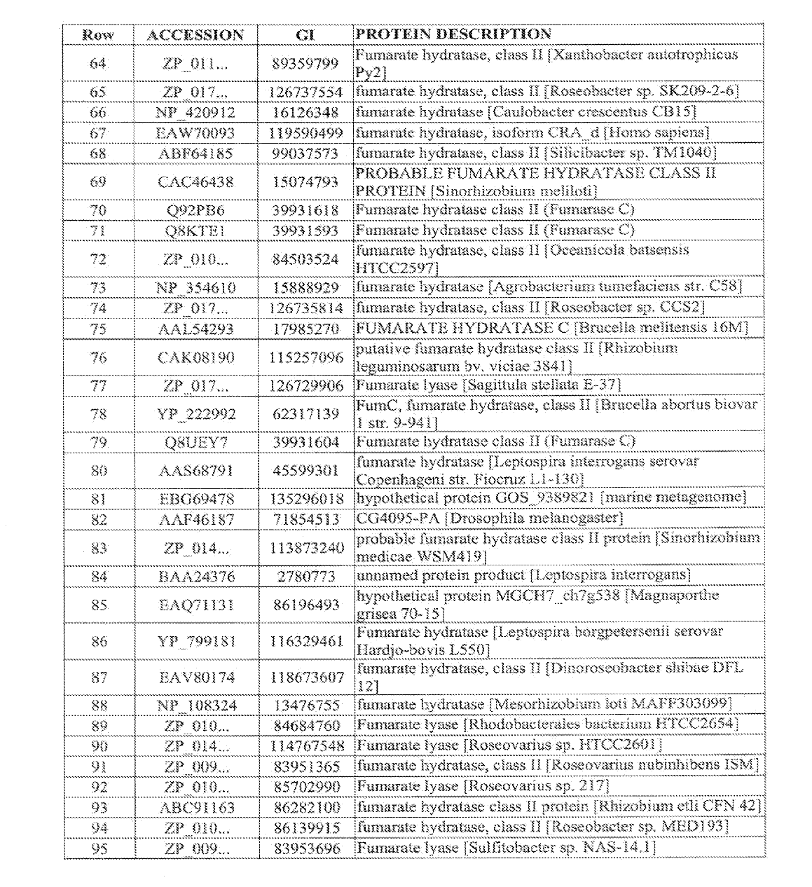

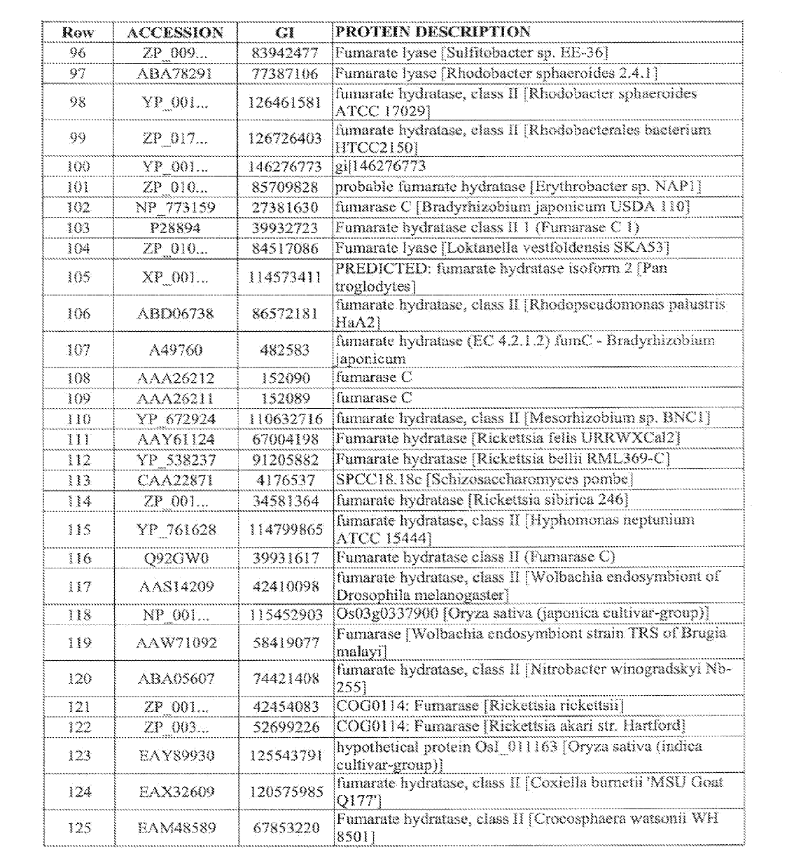

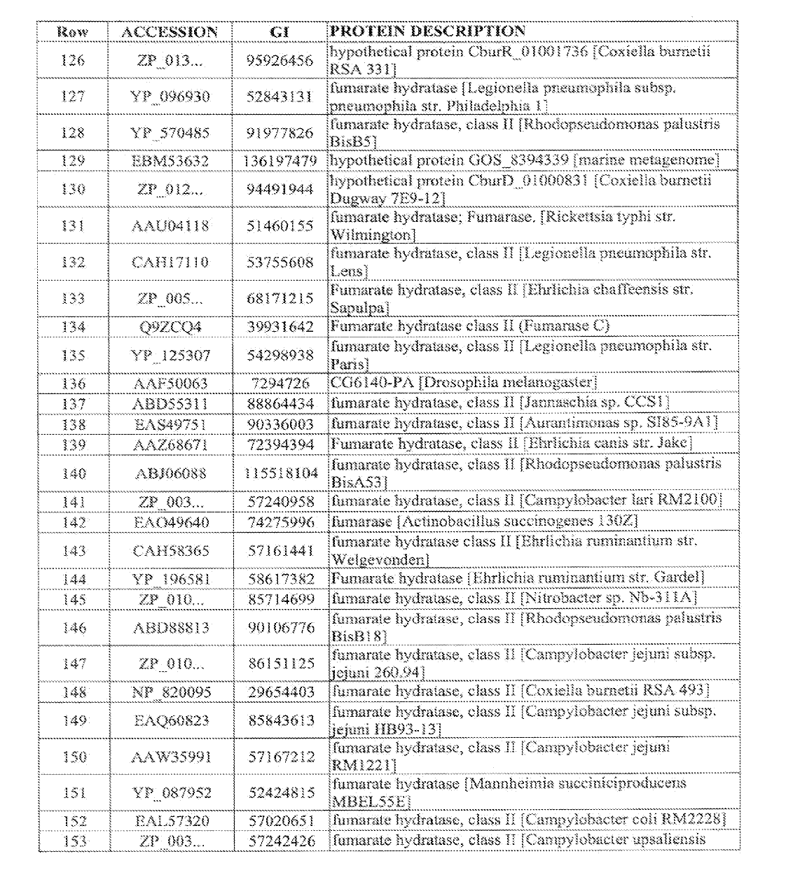

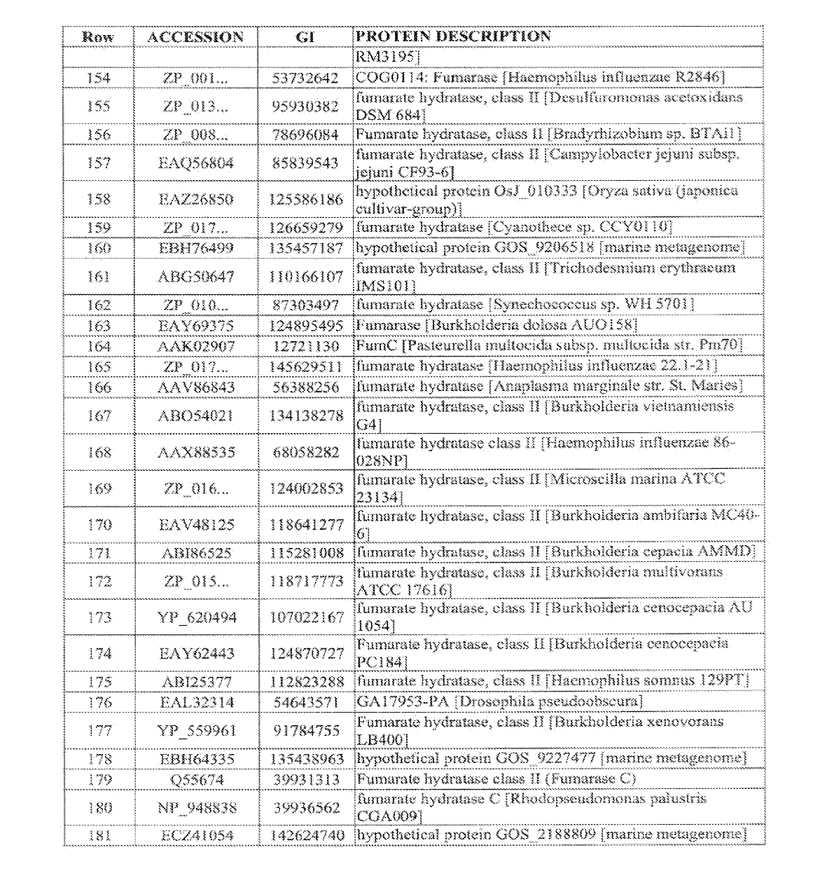

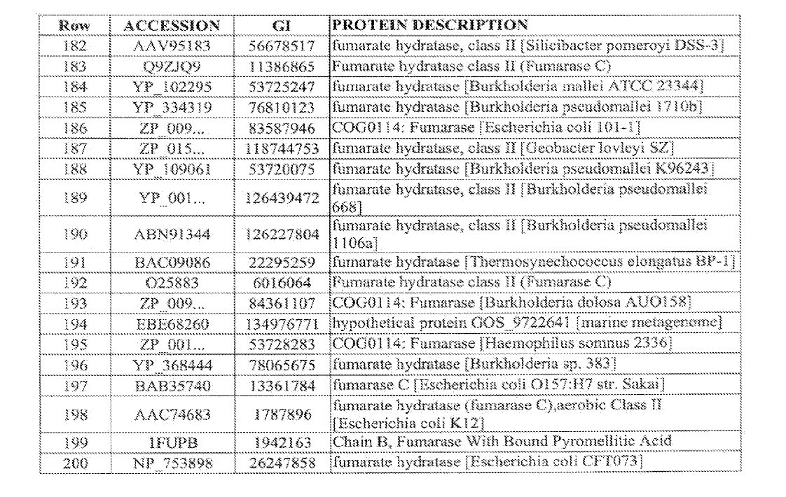

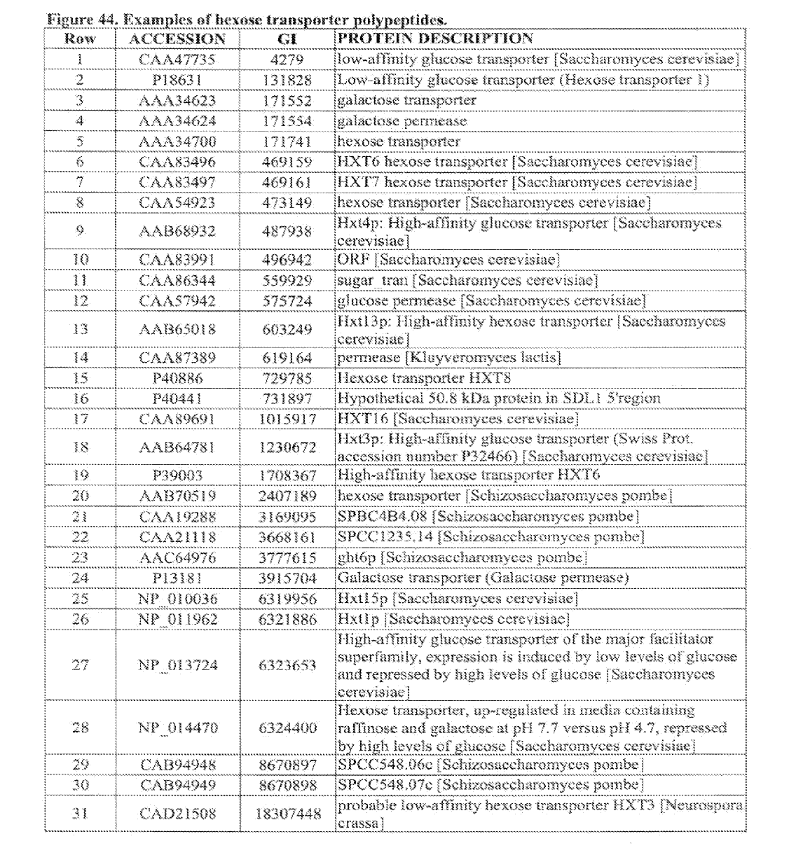

Hexose transporter (HXT) polypeptides: “Hexose transporter (HXT) polypeptides” are proteins that belong to the major facilitator superfamily (MFS) of transporters. HXT polypeptides transport their substrates by passive, energy-independent facilitated diffusion, with glucose moving down a concentration gradient. Many prokaryotic and eukaryotic, including mammalian, sugar transporters are of the MFS superfamily. The genome of the yeast S. cerevisiae encodes at least 20 candidate HXT polypeptides, while seven (encoded by the HXT1 through HXT7 genes) have been demonstrated to encode functional glucose transporters. Expression of any one of these HXT polypeptides in a parent strain otherwise lacking the HXT1 through HXT7 genes is sufficient to facilitate growth on a medium with glucose as the sole carbon source. HXT2, HXT6, and HXT7 polypeptides are believed to be high-affinity glucose transporters, whereas HXT3 and HXT4 polypeptides are low-affinity glucose transporters. Examples of hexose transporter (HXT) polypeptides are represented by the Genbank GI numbers in FIG. 44 and the hexose transporter polypeptides in FIG. 29. In some cases, a hexose transporter (HXT) polypeptides has an amino acid sequence that is at least about 75%, 85%, 90%, 95% or 100% identical to that of a polypeptide represented by the Genbank GI numbers in FIG. 44 or a hexose transporter polypeptide in FIG. 29.

Homologous: The term “homologous”, as used herein, means from the same source organism as the host cell. For example, as used here to refer to genetic material or to polypeptides, the term “homologous” refers to genetic material or polypeptides that naturally occurs in the organism in which it is present and/or being expressed, although optionally at different activity levels and/or in different amounts.

Host cell: As used herein, the “host cell” is a cell that is manipulated according to the present invention to increase production of one or more organic acids as described herein. A “modified host cell”, as used herein, is any host cell which has been modified, engineered, or manipulated in accordance with the present invention as compared with a parental cell. In some embodiments, the parental cell is a naturally occurring parental cell. Typically, the host cell is a microbial cell such as a fungal cell or a yeast cell.

Hybridization: “Hybridization” refers to the ability of a strand of nucleic acid to join with a complementary strand via base pairing. Hybridization occurs when complementary sequences in the two nucleic acid strands bind to one another.

Isocitrate lyase polypeptides: “Isocitrate lyase polypeptides” are polypeptides that catalyze the formation of succinate and glyoxylate from isocitrate (EC 4.1.3.1), a key reaction of the glyoxylate cycle. Examples of isocitrate lyase polypeptides are represented by the Genbank GI numbers in FIG. 49 and the isocitrate lyase polypeptides in FIG. 29. In some cases, an isocitrate lyase polypeptide has an amino acid sequence that is at least about 75%, 85%, 90%, 95% or 100% identical to that of a polypeptide represented by the Genbank GI numbers in FIG. 49 or an isocitrate lyase polypeptide in FIG. 29.

Isolated: The term “isolated”, as used herein, means that the isolated entity has been separated from at least one component with which it was previously associated. When most other components have been removed, the isolated entity is “purified” or “concentrated”. Isolation and/or purification and/or concentration may be performed using any techniques known in the art including, for example, fractionation, extraction, precipitation, or other separation.

Malate dehydrogenase polypeptide: A malate dehydrogenase (MDH) polypeptide is any enzyme capable of catalyzing the introconversion of oxaloacetate to malate (using NAD(P)+) and vice versa (EC 1.1.1.37). Malate dehydrogenase polypeptides can be localized to the mitochondria or to the cystosol. In some embodiments, the MDH is active in the cytosol. In some embodiments, the MDH polypeptide retains activity in the presence of glucose. In some embodiments, activity of the MDH polypeptide in the presence of glucose is at least 20%, 30%, 40%, 50%, 60%, 70%, 80%, 90%, 95%, 96%, 97%, 98%, 99%, 99%, or 100% of that observed under otherwise identical activity in the absence of glucose. Such an MDH polypeptide is considered “not inactivated” in the presence of glucose. Examples of MDH polypeptides are represented by the Genbank GI numbers in FIG. 34 and the malate dehydrogenase polypeptides in FIG. 29. In some cases, a MDH polypeptide has an amino acid sequence that is at least about 75%, 85%, 90%, 95% or 100% identical to that of a polypeptide represented by the Genbank GI numbers in FIG. 34 or a malate dehydrogenase polypeptide in FIG. 29. In one embodiment, an MDH polypeptide for use in accordance with the present invention contains a signaling sequence or sequences capable of targeting the MDH polypeptide to the cytosol of the yeast, or the MDH polypeptide lacks a signaling sequence or sequences capable of targeting the MDH polypeptide to an intracellular region of the yeast other than the cytosol. In one embodiment, the MDH polypeptide can be S. cerevisiae MDH3ΔSKL, in which the coding region encoding the MDH has been altered to delete the carboxy-terminal SKL residues of wild type S. cerevisiae MDH3, which normally target the MDH3 to the peroxisome.

Malate synthase polypeptides: “Malate synthase polypeptides” are enzymes of the glyoxylate cycle that catalyze the irreversible condensation of acetyl-CoA and glyoxylate to yield malate and CoA (EC 2.3.3.9). Malate synthase polypeptide activities, like those of isocitrate lyase polypeptides, are typically elevated when a non-fermentable carbon source is provided. Examples of malate synthase polypeptides are represented by the Genbank GI numbers in FIG. 37 and the malate synthase polypeptides in FIG. 29. In some cases, a malate synthase polypeptide has an amino acid sequence that is at least about 75%, 85%, 90%, 95% or 100% identical to that of a polypeptide represented by the Genbank GI numbers in FIG. 37 or a malate synthase polypeptide in FIG. 29.

Malic enzyme polypeptides: “Malic enzyme polypeptides” are polypeptides that catalyze the reversible NAD-dependent or NADP-dependent (EC 1.1.1.40) oxidative decarboxylation of (EC 1.1.1.38 or 1.1.1.39) malate to carbon dioxide and pyruvate, with the concomitant reduction of NAD(P). The enzyme is found in most living organisms, because the products of the reaction are used as a source of carbon and reductive power in different cell compartments. Most fungi encode a NADP-dependent malic enzyme. In S. cerevisiae, the malic enzyme polypeptide is encoded by the MAE1 gene. Examples of malic enzyme polypeptides are represented by the Genbank GI numbers in FIG. 38 and the malic enzyme polypeptides in FIG. 29. In some cases, a malic enzyme polypeptide has an amino acid sequence that is at least about 75%, 85%, 90%, 95% or 100% identical to that of a polypeptide represented by the Genbank GI numbers in FIG. 38 or a malic enzyme polypeptide in FIG. 29.

Medium: As is known in the art, the term “medium” refers to a chemical environment in which a host cell, such as a microbial cell (e.g., a yeast or fungal cell) is cultivated. Typically, a medium contains components required for the growth of the cell, and one or more precursors for the production of a dicarboxylic acid. Components for growth of host cells and precursors for the production of a dicarboxylic acid may or may be not identical.

Modified: The term “modified”, as used herein, refers to a host cell that has been modified to increase production of one or more organic acids, as compared with an otherwise identical host organism that has not been so modified. In principle, such “modification” in accordance with the present invention may comprise any chemical, physiological, genetic, or other modification that appropriately alters production of an organic acid in a host organism as compared with such production in an otherwise identical cell not subject to the same modification. In most embodiments, however, the modification will comprise a genetic modification. For example, a genetic modification can entail: the addition of all or a portion of gene that is not naturally present in the host cell, the addition of all or a portion of a gene that is already present in the host cell, the deletion of all or a portion of a gene that is naturally in the host cell, an alteration (e.g., a sequence change in) a gene that is naturally present in the host cell (e.g., a sequence change that increases expression, a sequence change that decreases expression, a sequence change that increases enzymatic, transport or other activity of a polypeptide, a sequence change that decreases enzymatic, transport or other activity of a polypeptide) and combinations thereof. In some cases, a modification comprises at least one chemical, physiological, genetic, or other modification; in other cases, a modification comprises more than one chemical, physiological, genetic, or other modification. In certain aspects where more than one modification is utilized, such modifications can comprise any combination of chemical, physiological, genetic, or other modification (e.g., one or more genetic, chemical and/or physiological modification(s)).

Open reading frame: As is known in the art, the term “open reading frame (ORF)” refers to a region of DNA or RNA encoding a peptide, polypeptide, or protein.

Organic acid compound: As used herein, the term “organic acid compound” can refer to any of a variety of organic acids. In certain embodiments, the term refers to C4 dicarboxylic acid compounds. Representative organic acids include, for example, fumaric acid, malic acid, succinic acid, tartaric acid, and combinations thereof.

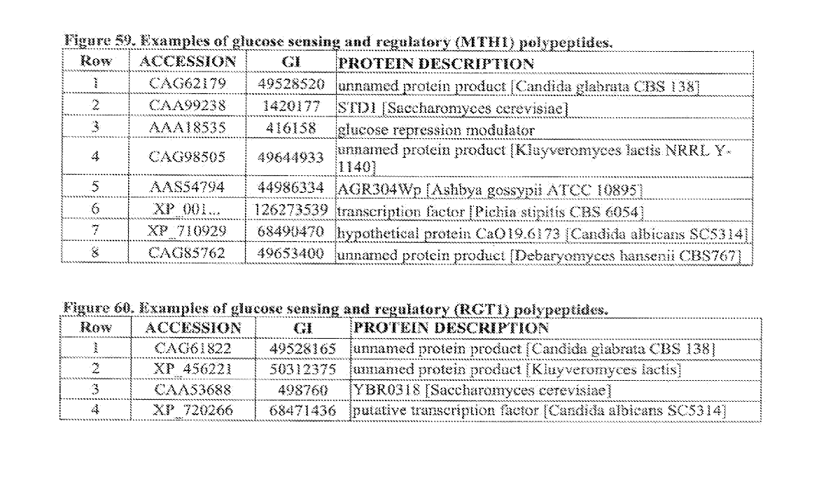

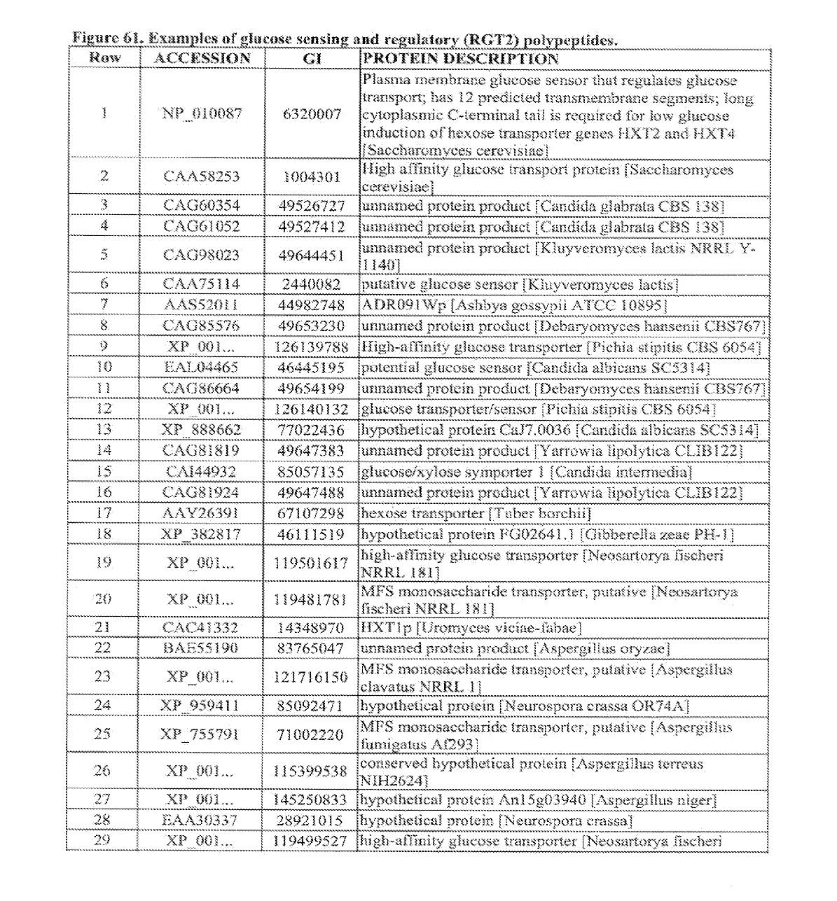

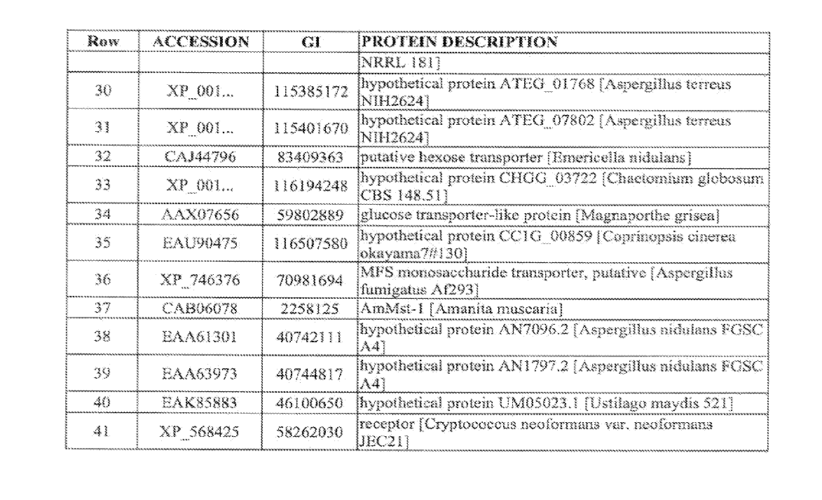

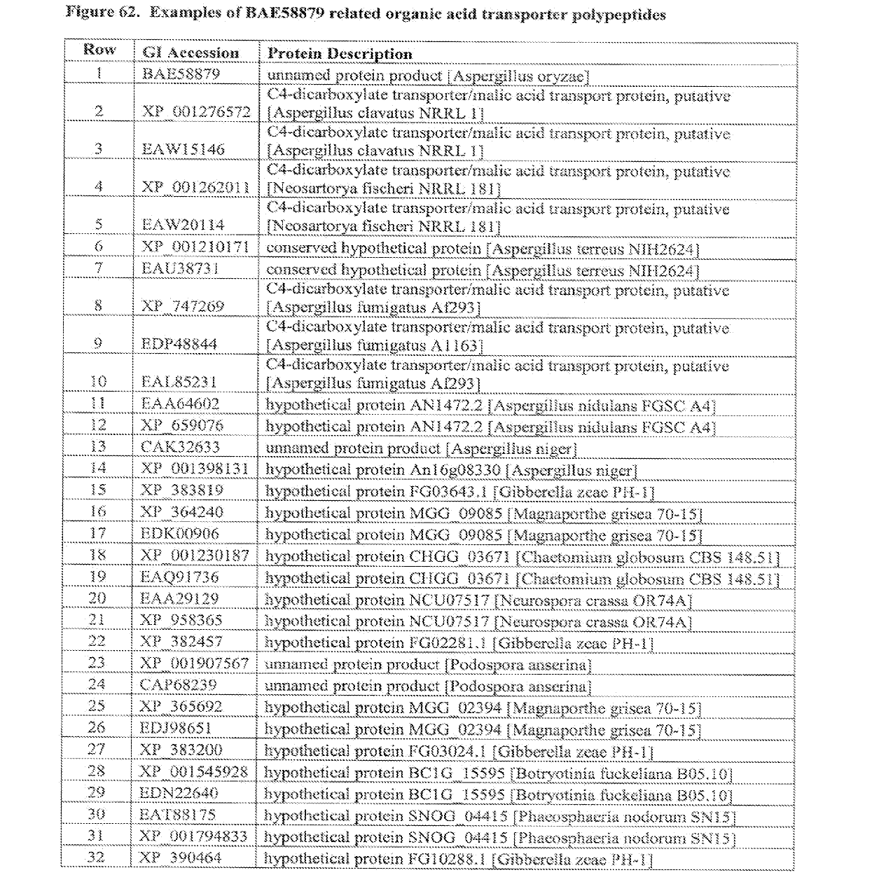

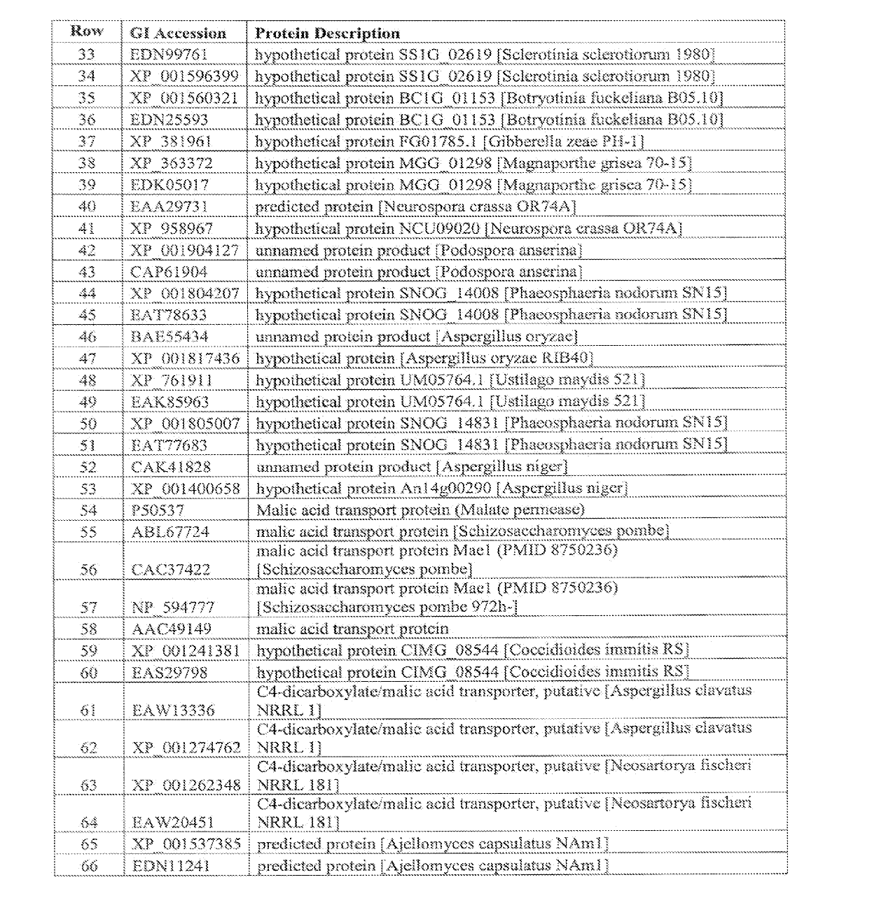

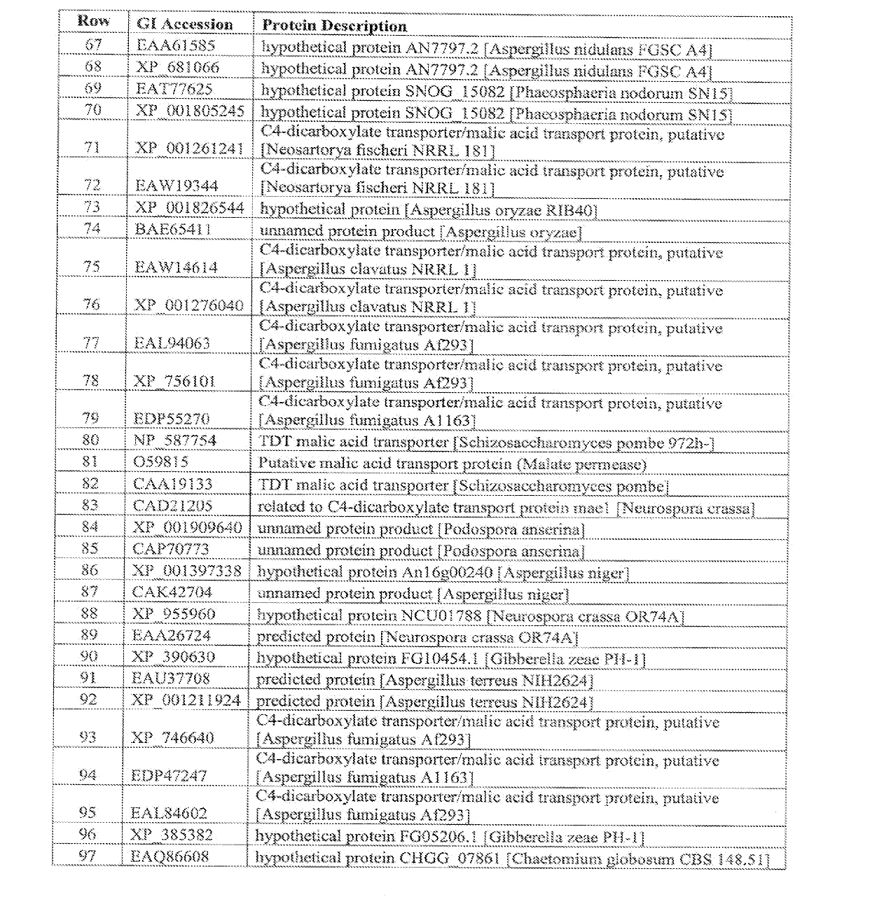

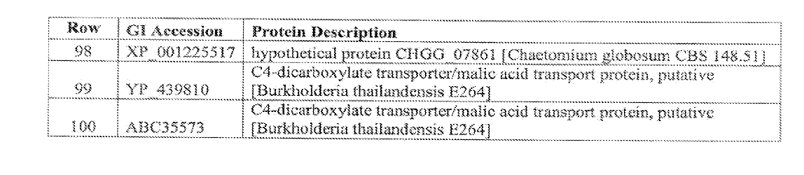

Organic acid transporter (OAT) polypeptides: “Organic acid transporter (OAT) polypeptides”, which include but are not limited to malic acid transporter (MAE) polypeptides, are proteins whose expression and/or activities can be modified to catalyze the net efflux of one or more dicarboxylic acids from fungal cells. OAT polypeptides are a diverse set of proteins that catalyze carboxylic acid transport via several distinct mechanisms. The activity of a particular OAT polypeptide may be either increased or reduced, depending on the substrate(s) for a given OAT polypeptide and the desired dicarboxylic acid product. Furthermore, it may be possible to modify the subcellular localization of an OAT polypeptide to promote the efflux of a specific dicarboxylic acid product. As an example, a vacuolar or tonoplast dicarboxylate transporter may be targeted to the cytoplasmic membrane in order to facilitate the efflux of a dicarboxylic acid product such as malic acid. Representative OAT polypeptides include the S. pombe malate transporter MAE1, aluminum activated malate transporters (e.g. ALMT1), plant tonoplast dicarboxylate transporters (e.g. A. thaliana AttDT), mammalian sodium/dicarboxylate co-transporters, mono- and dicarboxylic acid transporters related to the K. lactis JEN1 and JEN2 proteins, respectively; and proteins related to the E. coli DcuC succinate efflux polypeptide. Examples of OAT polypeptides are represented by the Genbank GI numbers in FIG. 35 and the OAT polypeptides in FIG. 29. In some cases, an OAT polypeptide has an amino acid sequence that is at least about 75%, 85%, 90%, 95% or 100% identical to that of a polypeptide represented by the Genbank GI numbers in FIG. 35 or FIG. 62 or an OAT polypeptide in FIG. 29 or an A. oryzae transporter. A. oryzae

A “malic acid transporter protein (MAE) polypeptide” can be any protein capable of transporting malate from the cytosol of a yeast across the cell membrane and into extracellular space. A protein need not be identified in the literature as a malic acid transporter protein at the time of filing of the present application to be within the definition of an MAE.

An MAE from any source organism may be used and the MAE may be wild type or modified from wild type. The MAE can be Schizosaccharomyces pombe SpMAE1. In one embodiment, the MAE has at least 75% identity to the amino acid sequence given in SEQ ID NO:3. In one embodiment, the MAE has at least 80% identity to the amino acid sequence given in SEQ ID NO:3. In one embodiment, the MAE has at least 85% identity to the amino acid sequence given in SEQ ID NO:3. In one embodiment, the MAE has at least 90% identity to the amino acid sequence given in SEQ ID NO:3. In one embodiment, the MAE has at least 95% identity to the amino acid sequence given in SEQ ID NO:3. In another embodiment, the MAE has at least 96% identity to the amino acid sequence given in SEQ ID NO: 3. In an additional embodiment, the MAE has at least 97% identity to the amino acid sequence given in SEQ ID NO: 3. In yet another embodiment, the MAE has at least 98% identity to the amino acid sequence given in SEQ ID NO: 3. In still another embodiment, the MAE has at least 99% identity to the amino acid sequence given in SEQ ID NO: 3. In still yet another embodiment, the MAE has the amino acid sequence given in SEQ ID NO: 3. A. oryzae

Another useful transporter is the A. oryzae transporter. Useful transporters can have an amino acid sequence that is at least 80%, 85%, 87%, 89%, 90%, 93%, 95%, 98% or 99% identical to the A. oryzae transporter or SEQ ID NO: ______ or is identical to the amino acid sequence of SEQ ID NO:______.

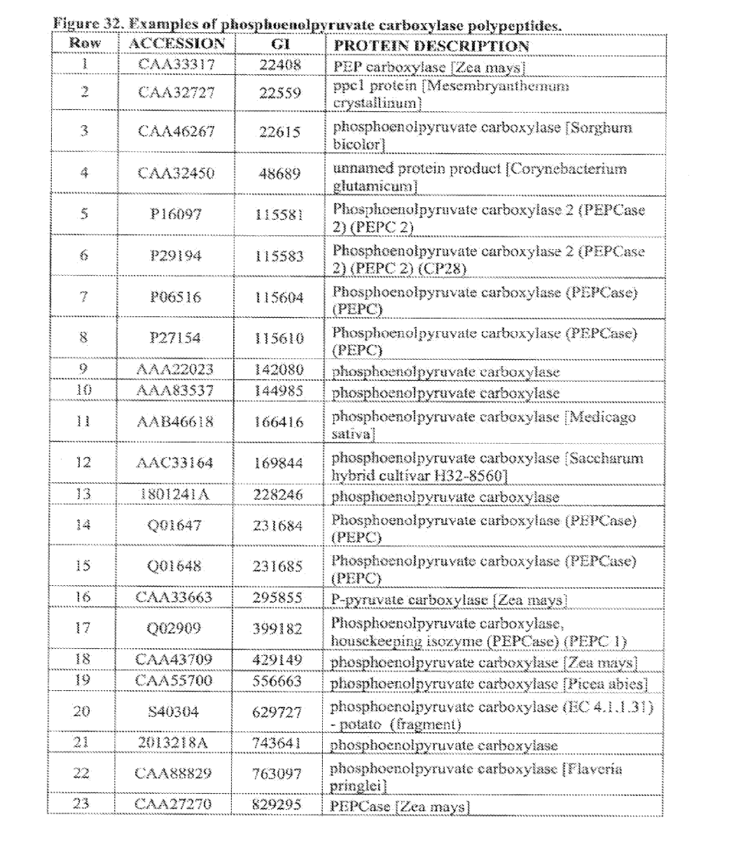

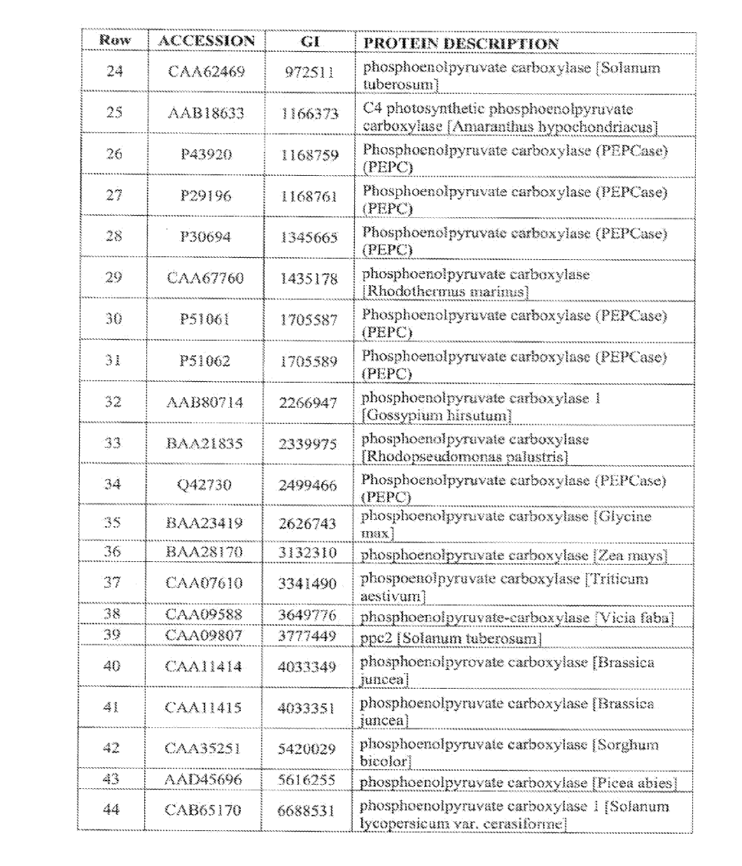

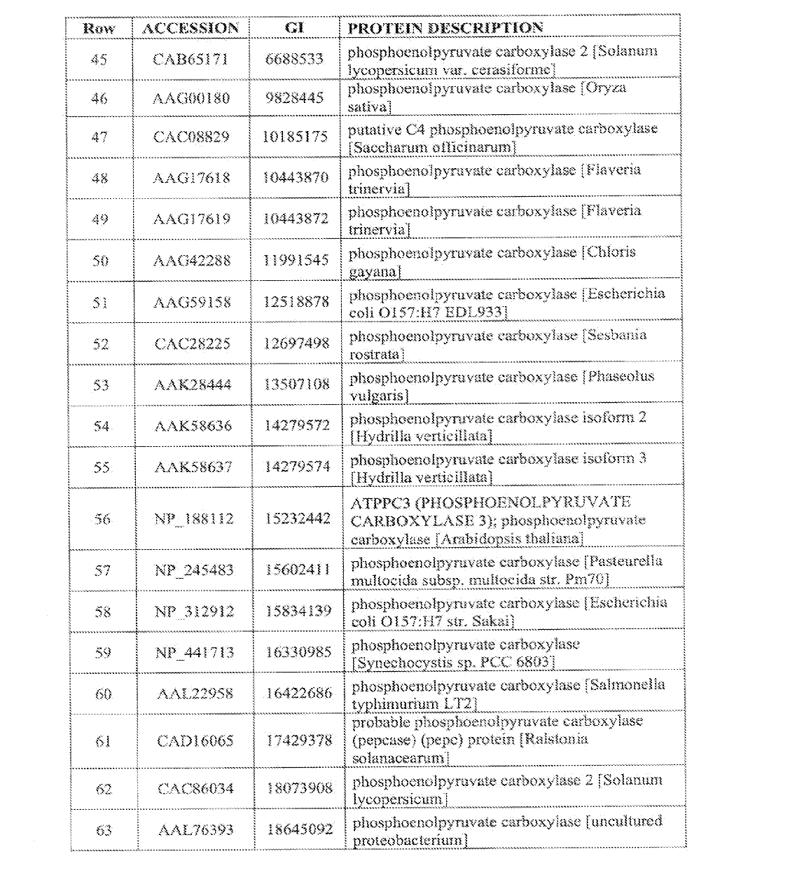

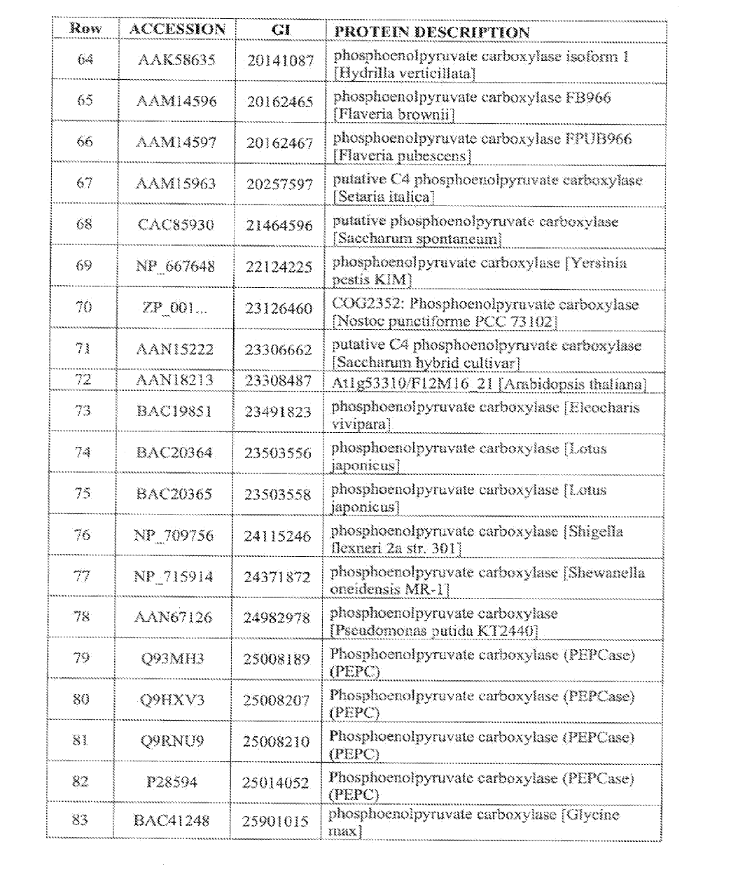

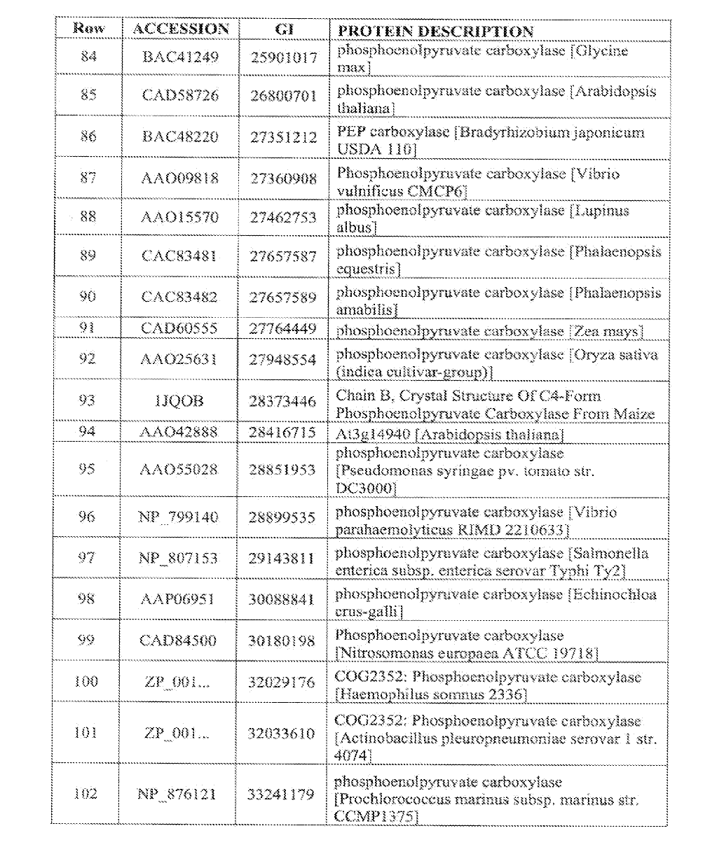

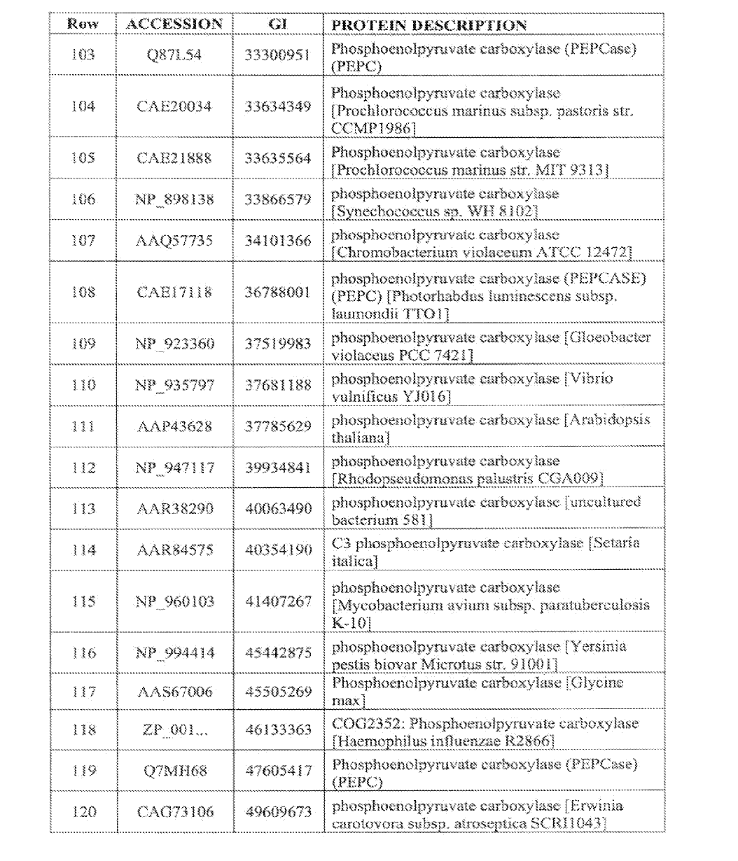

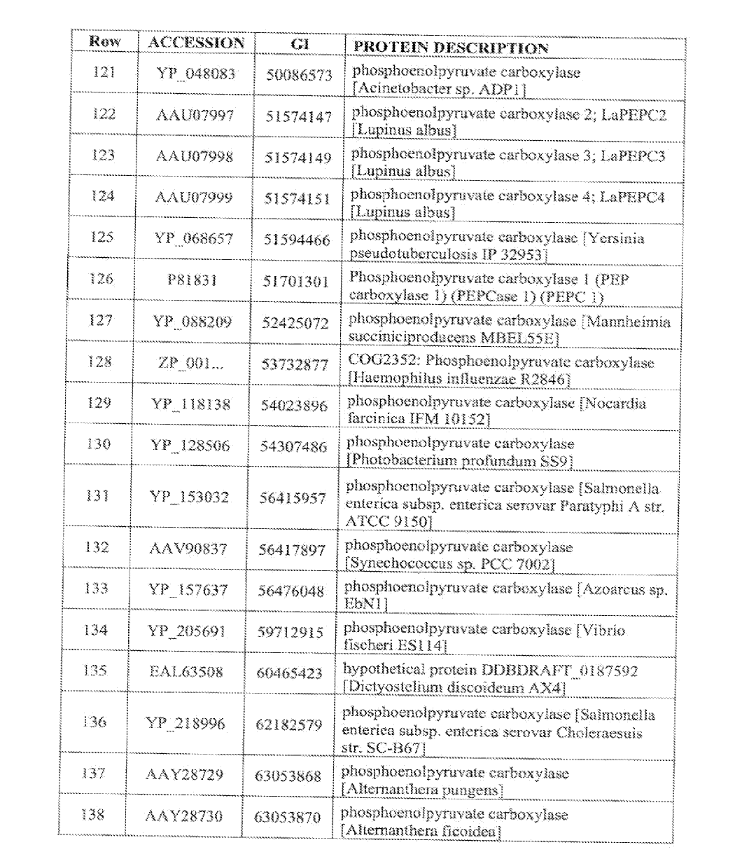

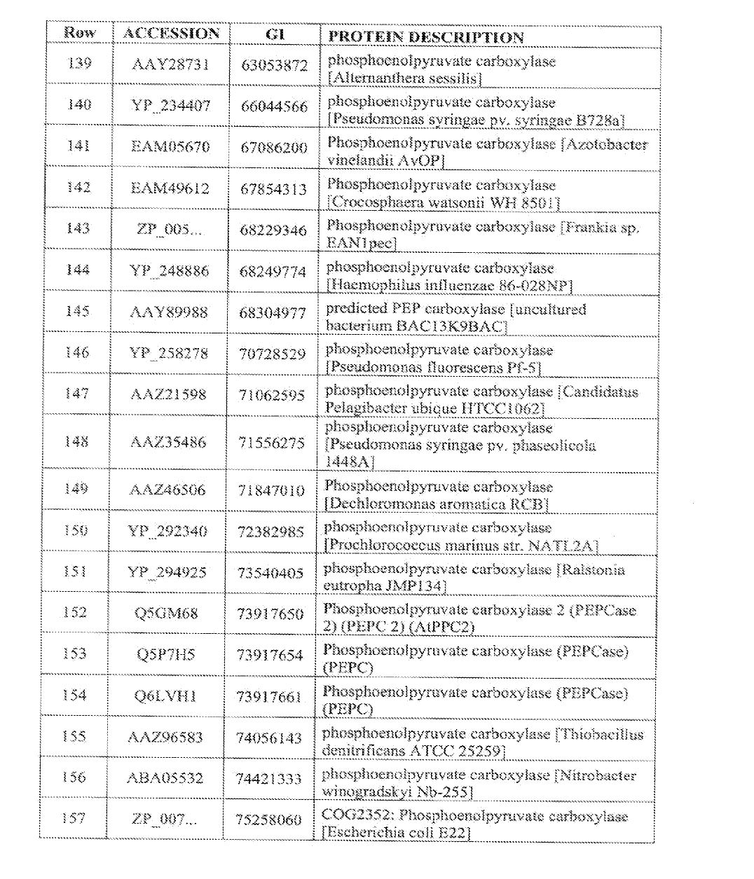

PDC-reduced: As used herein, the term “PDC-reduced” refers to a yeast cell containing a modification (e.g., a genetic modification that deletes all or a portion of a PDC gene or a genetic modification that alters the activity or expression of PDC) that reduces pyruvate decarboxylase activity as compared with an otherwise identical yeast that is not modified. In some embodiments, a PDC-reduced yeast cell has reduced activity of one or more pyruvate decarboxylase polypeptides relative to the unmodified yeast cell (e.g., an otherwise identical yeast cell lacking the modification). In certain cases thereof the pyruvate decarboxylase polypeptide is chosen from one or more of Pdc1, Pdc2, Pdc5, Pdc6 polypeptides including any of the pyruvate decarboxylase and Pdc2 polypeptides in FIG. 31. In some cases, a PDC-reduced cell has reduced or substantially eliminated Pdc1 polypeptide activity. In certain cases, the PDC-reduced cell further comprises reduced or substantially eliminated Pdc2, Pdc5, and/or Pdc6 polypeptide activity. In some cases, a PDC-reduced cell has reduced or substantially eliminated Pdc2 polypeptide activity. In certain embodiments thereof, the PDC-reduced cell further comprises reduced or substantially eliminated Pdc1, Pdc5, and/or Pdc6 polypeptide activity. In some cases, a PDC-reduced cell has reduced or substantially eliminated Pdc5 polypeptide activity. In certain cases thereof, the PDC-reduced cell further comprises reduced and/or substantially eliminated Pdc1, Pdc2, and/or Pdc6 polypeptide activity. In some cases a PDC-reduced cell has reduced or substantially eliminated Pdc6 polypeptide activity. In certain cases, the PDC-reduced cell further comprises reduced and/or substantially eliminated Pdc1, Pdc2, and/or Pdc5 polypeptide activity. In some cases a PDC-reduced cell has reduced and/or substantially eliminated Pdc1 and Pdc5 polypeptide activity. In some cases, a PDC-reduced cell has reduced and/or substantially eliminated Pdc1 and Pdc6 polypeptide activity. In some cases, a PDC-reduced cell has reduced and/or substantially eliminated Pdc5 and Pdc6 polypeptide activity. In some cases, a PDC-reduced cell has reduced and/or substantially eliminated Pdc1, Pdc5 and Pdc6 polypeptide activity. In some embodiments, a PDC-reduced cell has 3-fold, 5-fold, 10-fold, 50-fold less pyruvate decarboxylase activity as compared with an otherwise identical parental cell not containing the modification. In some cases, a PDC-reduced cell has pyruvate decarboxylase activity below at least about 0.075 micromol/min mg protein⁻¹, at least about 0.045 micromol/min mg protein⁻¹, at least about 0.025 micromol/min mg protein⁻¹; in some embodiments, a PDC-reduced cell has pyruvate decarboxylase activity below about 0.005 micromol/min mg protein⁻¹ when using the methods described by van Maris et al. (Overproduction of Threonine Aldolase Circumvents the Biosynthetic Role of Pyruvate Decarboxylase in Glucose-grown Saccharomyces cerevisiae. Appl. Environ. Microbiol. 69:2094-2099, 2003). In some cases, a PDC-reduced cell has no detectable pyruvate decarboxylase activity. In some cases, a cell with no detectable pyruvate decarboxylase activity is referred to as “PDC-negative”. In some cases, a PDC-negative cell lacks Pdc1, Pdc5 and Pdc6 polypeptide activity. In some cases, a PDC-negative cell has pyruvate decarboxylase activity below about 0.005 micromol/min mg protein⁻¹.

Phosphoenolpyruvate carboxykinase (PCK) polypeptide: A “phosphoenolpyruvate carboxykinase (PCK) polypeptide” is a polypeptide that catalyzes the reversible formation of oxaloacetate and ATP from phosphoenolpyruvate, ADP, and carbon dioxide (EC 4.1.1.49). Under physiological conditions such as glucose limitation, PCK acts to catalyze the formation of phosphoenolpyruvate from OAA (for gluconeogenesis), thereby reversing the anaplerotic flux provided by PYC and PPC. Examples of PCK polypeptides are represented by the Genbank GI numbers in FIG. 36 and the PCK polypeptides in FIG. 29. In some cases, a PCK polypeptide has an amino acid sequence that is at least about 75%, 85%, 90%, 95% or 100% identical to that of a polypeptide represented by the Genbank GI numbers in FIG. 36 or a PCK polypeptide in FIG. 29.

Phosphoenolpyruvate carboxylase (PPC) polypeptide: A “phosphoenolpyruvate carboxylase (PPC) polypeptide” is a polypeptide catalyzes the addition of carbon dioxide to phosphoenolpyruvate (PEP) to form oxaloacetate (EC 4.1.1.31). E. coli PPC has been observed to be negatively regulated by downstream products including by malate. In some embodiments, the PPC polypeptide is modified to be less sensitive to inhibition by one or more of malate, aspartate, and/or oxaloacetate. Examples of PPC polypeptides are represented by the Genbank GI numbers in FIG. 32 and the PPC polypeptides in FIG. 29. In some cases, a PPC polypeptide has an amino acid sequence that is at least about 75%, 85%, 90%, 95% or 100% identical to that of a polypeptide represented by the Genbank GI numbers in FIG. 32 or a PPC polypeptide in FIG. 29.

Plasmid: As is known in the art, the term “plasmid” refers to a circular or linear, extra-chromosomal, replicatable piece of DNA.

Polymerase chain reaction: As is known in the art, the term “polymerase chain reaction (PCR)” refers to an enzymatic technique to create multiple copies of one sequence of nucleic acid. Copies of DNA sequence are prepared by shuttling a DNA polymerase between two amplimers. The basis of this amplification method is multiple cycles of temperature changes to denature, then re-anneal amplimers, followed by extension to synthesize new DNA strands in the region located between the flanking amplimers.

Polypeptide: The term “polypeptide”, as used herein, generally has its art-recognized meaning of a polymer of at least three amino acids. However, the term is also used to refer to specific functional classes of polypeptides, such as, for example, anaplerotic polypeptides (e.g. pyruvate carboxylase (PYC) polypeptides, phosphoenolpyruvate carboxylase (PPC) polypeptides, phosphoenolpyruvate carboxykinase (PCK) polypeptides, pyruvate kinase (PYK) polypeptides, biotin protein ligase activity (BPL) polypeptides, biotin transport protein (VHT) polypeptides, bicarbonate transport polypeptides, and carbonic anhydrase polypeptides), C4-dicarboxylic acid biosynthetic polypeptides (e.g., ATP-citrate lyase polypeptides, fumarase polypeptides, fumarate reductase polypeptides, isocitrate lyase polypeptides, malate synthase polypeptides, malate dehydrogenase, malic enzyme polypeptides, and/or succinate dehydrogenase polypeptides), GSR polypeptides (e.g. SNF1, MIG1, MIG2, HXK2, RGT1, SNF3, RGT2, STD1, MTH1, GRR1, YCK1, HXK1, and GLK1), hexose transporter polypeptides (e.g. HXT1, HXT2, HXT3, HXT4, HXT5, HXT6, and HXT7), organic acid transporter (OAT) polypeptides (e.g. S. pombe malate transporter MAE1, aluminum activated malate transporters (e.g. ALMT1), plant tonoplast dicarboxylate transporters (e.g. A. thaliana AttDT), mammalian sodium/dicarboxylate co-transporters, mono- and dicarboxylic acid transporters related to the K. lactis JEN1 and JEN2 proteins, A. oryzae transporter polypeptidesrespectively; and proteins related to the E. coli DcuC succinate efflux polypeptide), and pyruvate decarboxylase (PDC) polypeptides. For each such class, the present specification provides several examples of known sequences of such polypeptides. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented in FIGS. 29 and FIGS. 31-61 herein. In some cases a polypeptide has an amino acid sequence that differs from the amino acid sequence of a polypeptide presented in the FIGS. 29 and FIGS. 31-61 herein by fewer than 20, 15, 10 or 5 amino acids. In some cases the amino acid changes are conservative changes.

Promoter: As is known in the art, the term “promoter” or “promoter region” refers to a DNA sequence, usually found upstream (5′) to a coding sequence, that controls expression of the coding sequence by controlling production of messenger RNA (mRNA) by providing the recognition site for RNA polymerase and/or other factors necessary for start of transcription at the correct site.

Pyruvate carboxylase enzyme (PYC) polypeptide: A “pyruvate carboxylase (PYC) polypeptide” can be any enzyme that uses a HCO₃⁻ substrate to catalyze an ATP-dependent conversion of pyruvate to oxaloacetate (EC 6.4.1.1). PYC polypeptides contain a covalently attached biotin prosthetic group, which serves as a carrier of activated CO₂. In most instances, the activity of PYC polypeptides depends on the presence of acetyl-CoA. Biotin is not carboxylated (on PYC) unless acetyl-CoA (or a closely related acyl-CoA) is bound to the enzyme. Aspartate often serves as an inhibitor of PYC polypeptides. PYC polypeptides are generally active in a tetrameric form. Examples of pyruvate carboxylase polypeptides are represented by the Genbank GI numbers in FIG. 33 and the pyruvate carboxylase polypeptides in FIG. 29. In some cases, a pyruvate carboxylase has an amino acid sequence that is at least about 75%, 85%, 90%, 95% or 100% identical to that of a polypeptide represented by the Genbank GI numbers in FIG. 33 or a pyruvate carboxylase polypeptide in FIG. 29.

In one embodiment, a PYC polypeptide is a PYC that has at least 75% identity to the amino acid sequence given in SEQ ID NO:1. In one embodiment, the PYC has at least 80% identity to the amino acid sequence given in SEQ ID NO:1. In one embodiment, the PYC has at least 85% identity to the amino acid sequence given in SEQ ID NO:1. In one embodiment, the PYC has at least 90% identity to the amino acid sequence given in SEQ ID NO:1. In one embodiment, the PYC has at least 95% identity to the amino acid sequence given in SEQ ID NO:1. In another embodiment, the PYC has at least 96% identity to the amino acid sequence given in SEQ ID NO: 1. In an additional embodiment, the PYC has at least 97% identity to the amino acid sequence given in SEQ ID NO: 1. In yet another embodiment, the PYC has at least 98% identity to the amino acid sequence given in SEQ ID NO: 1. In still another embodiment, the PYC has at least 99% identity to the amino acid sequence given in SEQ ID NO: 1. In still yet another embodiment, the PYC has the amino acid sequence given in SEQ ID NO: 1.

Pyruvate decarboxylase (PDC) polypeptide: A “pyruvate decarboxylase polypeptide” can be any thiamin diphosphate-dependent enzyme that catalyses the decarboxylation of pyruvic acid to acetaldehyde and carbon dioxide (EC 4.1.1.1). Examples of pyruvate decarboxylase polypeptides are represented by the Genbank GI numbers in FIG. 31 and the pyruvate decarboxylase polypeptides in FIG. 29. In some cases, a pyruvate decarboxylase has an amino acid sequence that is at least about 75%, 85%, 90%, 95% or 100% identical to that of a polypeptide represented by the Genbank GI numbers in FIG. 31 or a pyruvate decarboxylase polypeptide in FIG. 29.

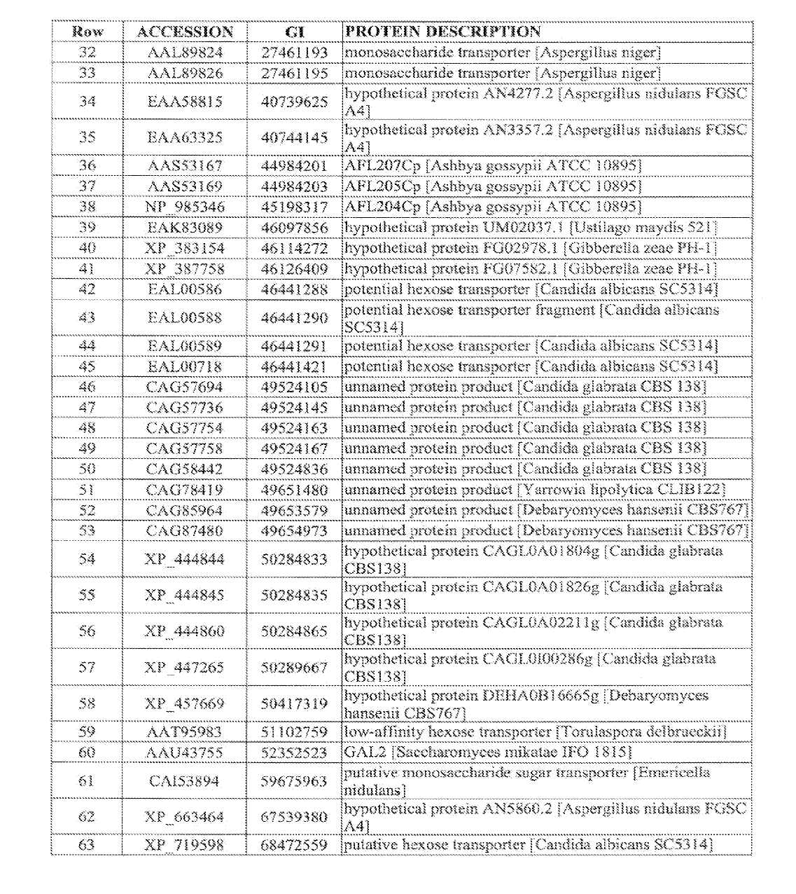

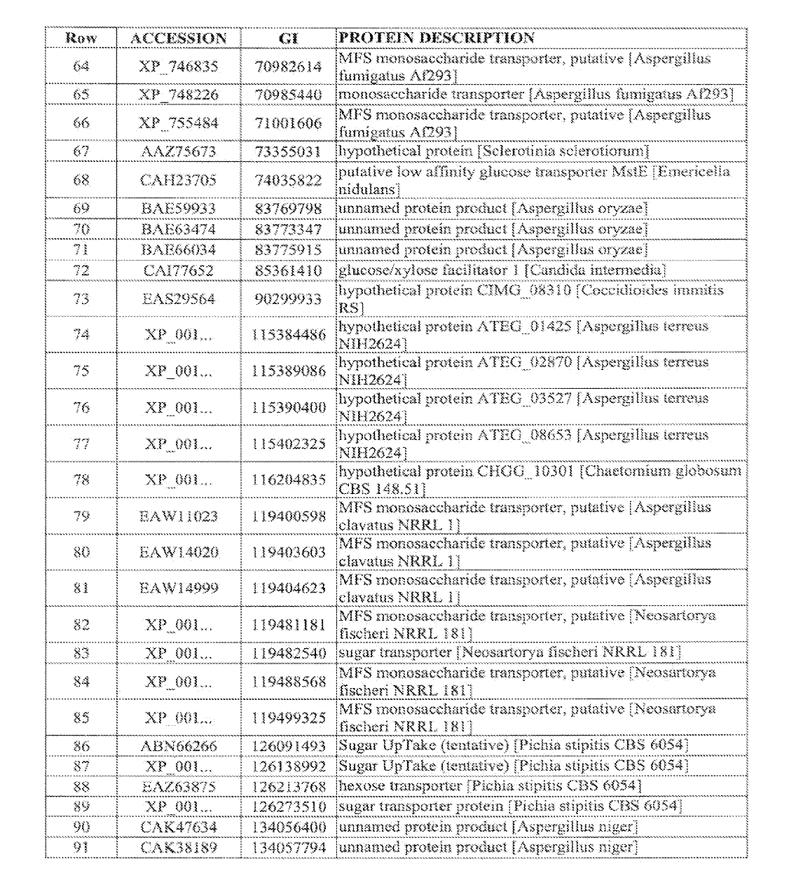

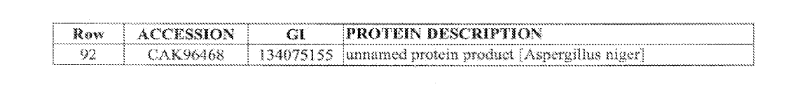

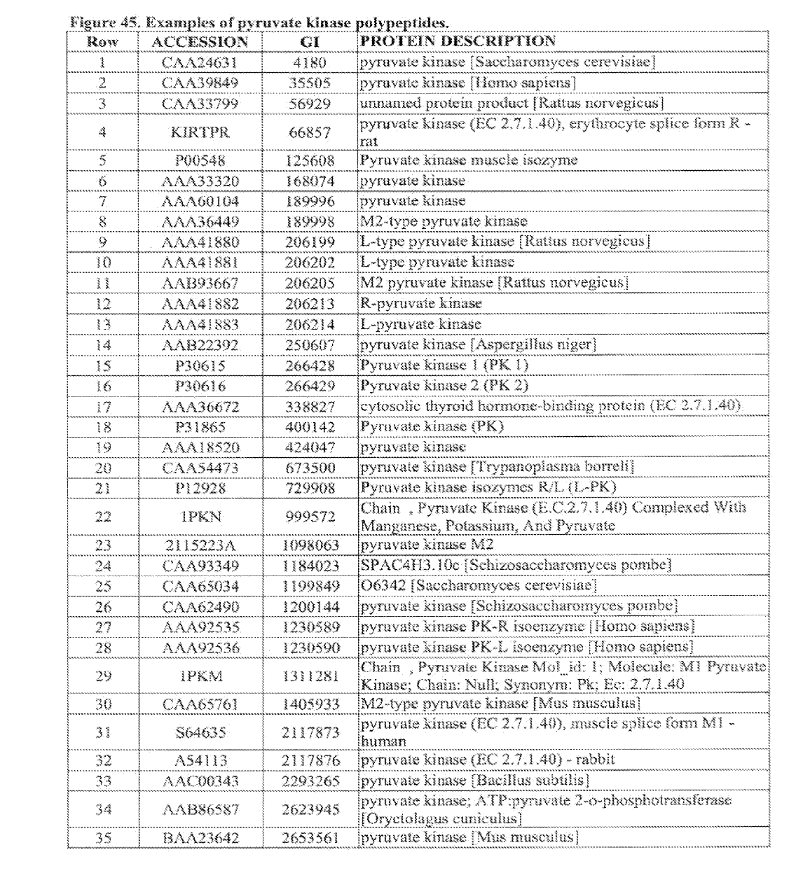

Pyruvate kinase: Pyruvate kinase catalyses the irreversible conversion of phosphoenolpyruvate (PEP) to pyruvate (EC 2.7.1.40), the final step in glycolysis. Many pyruvate kinase enzymes are tetrameric complexes of identical subunits. PYK polypeptides play a key role in regulating glycolytic flux. PYK polypeptides from Saccharomyces cerevisiae have an absolute requirement for both monovalent and divalent cations, undergo homotropic activation by PEP and Mn²⁺, and heterotropic activation by fructose 1,6-bisphosphate (FBP). Potassium is the physiologically important monovalent activator, but several other monovalent cations can also activate PYK polypeptides. Examples of pyruvate kinase polypeptides are represented by the Genbank GI numbers in FIG. 45 and the pyruvate kinase polypeptides in FIG. 29. In some cases, a pyruvate kinase has an amino acid sequence that is at least about 75%, 85%, 90%, 95% or 100% identical to that of a polypeptide represented by the Genbank GI numbers in FIG. 45 or a pyruvate kinase polypeptide in FIG. 29.

Recombinant: A “recombinant” host cell, as that term is used herein, is a host cell that has been genetically modified. For example, a “recombinant cell” can be a cell that contains a nucleic acid sequence not naturally occurring in the cell, or an additional copy or copies of an endogenous nucleic acid sequence, wherein the nucleic acid sequence is introduced into the cell or an ancestor thereof by human action. A recombinant cell includes, but is not limited to: a cell which has been genetically modified by deletion of all or a portion of a gene, a cell that has had a mutation introduced into a gene, a cell that has had a nucleic acid sequence inserted either to add a functional gene or disrupt a functional gene, and a cell that has a gene that has been modified by both removing and adding a nucleic acid sequence. A “recombinant vector” or “recombinant DNA or RNA construct” refers to any nucleic acid molecule generated by the hand of man. For example, a recombinant construct may be a vector such as a plasmid, cosmid, virus, autonomously replicating sequence, phage, or linear or circular single-stranded or double-stranded DNA or RNA molecule. A recombinant nucleic acid may be derived from any source and/or capable of genomic integration or autonomous replication where it includes two or more sequences that have been linked together by the hand of man. Recombinant constructs may, for example, be capable of introducing a 5′ regulatory sequence or promoter region and a DNA sequence for a selected gene product into a cell in such a manner that the DNA sequence is transcribed into a functional mRNA, which may or may not be translated and therefore expressed.

Restriction enzyme: As is known in the art, the term “restriction enzyme” refers to an enzyme that recognizes a specific sequence of nucleotides in double stranded DNA and cleaves both strands; also called a restriction endonuclease. Cleavage typically occurs within the restriction site or close to it.

Screenable: The term “screenable” is used to refer to a marker whose expression confers a phenotype facilitating identification, optionally without facilitating survival, of cells containing the marker. In many embodiments, a screenable marker imparts a visually or otherwise distinguishing characteristic (e.g. color changes, fluorescence).

Selectable: The term “selectable” is used to refer to a marker whose expression confers a phenotype facilitating identification, and specifically facilitating survival, of cells containing the marker. Selectable markers include those, which confer resistance to toxic chemicals (e.g. ampicillin, kanamycin) or complement a nutritional deficiency (e.g. uracil, histidine, leucine).

Sequence Identity: As used herein, the term “sequence identity” refers to a comparison between two sequences (e.g., two nucleic acid sequences or two amino acid sequences) and assessment of the degree to which they contain the same residue at the same position. As is known to those of ordinary skill in the art, an assessment of sequence identity includes an assessment of which positions in different sequences should be considered to be corresponding positions; adjustment for gaps etc. is permitted. Furthermore, an assessment of residue identity can include an assessment of degree of identity such that consideration can be given to positions in which the identical residue (e.g., nucleotide or amino acid) is not observed, but a residue sharing one or more structural, chemical, or functional features is found. Identity can be determined by a sequence alignment. The percent identity between the two sequences is a function of the number of identical positions shared by the sequences, taking into account the number of gaps, and the length of each gap, which needs to be introduced for optimal alignment of the two sequences. Any of a variety of algorithms or approaches may be utilized to calculate sequence identity. For example, in some embodiments, the Needleman and Wunsch (1970) J. Mol. Biol. 48:444-453 algorithm can be utilized. This algorithm has been incorporated into the GAP program in the GCG software package (available at http://www.gcg.com). In some such embodiments, the Neddleman and Wunsch algorithim is employed using either a Blossum 62 matrix or a PAM250 matrix, and a gap weight of 16, 14, 12, 10, 8, 6, or 4 and a length weight of 1, 2, 3, 4, 5, or 6. In some embodiments, sequence alignment is performed using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5. In some embodiments, a sequence alignment is performed using the algorithm of Meyers and Miller ((1989) CABIOS, 4:11-17). This algorithm has been incorporated into the ALIGN program (version 2.0). In some such embodiments, this algorithm is employed using a PAM120 weight residue table, a gap length penalty of 12 and a gap penalty of 4. In some embodiments, a sequence alignment is performed using the ClustalW program. In some such embodiments, default values, namely: DNA Gap Open Penalty=15.0, DNA Gap Extension Penalty=6.66, DNA Matrix=Identity, Protein Gap Open Penalty=10.0, Protein Gap Extension Penalty=0.2, Protein matrix=Gonnet, and employed. Identity can be calculated according to the procedure described by the ClustalW documentation: “A pairwise score is calculated for every pair of sequences that are to be aligned. These scores are presented in a table in the results. Pairwise scores are calculated as the number of identities in the best alignment divided by the number of residues compared (gap positions are excluded). Both of these scores are initially calculated as percent identity scores and are converted to distances by dividing by 100 and subtracting from 1.0 to give number of differences per site. In certain embodiments, the length of a sequence aligned for comparison purposes is at least 30%, at least 40%, at least 50%, at least 60%, at least 70%, at least 80%, at least 90%, at least 95% or 100% of the length of the reference sequence. The comparison of sequences and determination of percent identity between two sequences can be accomplished using a mathematical algorithm.

Small Molecule: In general, a small molecule is understood in the art to be an organic molecule that is less than about 5 kilodaltons (Kd) in size. In some embodiments, the small molecule is less than about 3 Kd, 2 Kd, or 1 Kd. In some embodiments, the small molecule is less than about 800 daltons (D), 600 D, 500 D, 400 D, 300 D, 200 D, or 100 D. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Source organism: The term “source organism”, as used herein, refers to the organism in which a particular polypeptide or genetic sequence can be found in nature. Thus, for example, if one or more homologous or heterologous polypeptides or genetic sequences is/are being expressed in a host organism, the organism in which the polypeptides or sequences are expressed in nature (and/or from which their genes were originally cloned) is referred to as the “source organism”. Where multiple homologous or heterologous polypeptides and/or genetic sequences are being expressed in a host organism, one or more source organism(s) may be utilized for independent selection of each of the heterologous polypeptide(s) or genetic sequences. It will be appreciated that any and all organisms that naturally contain relevant polypeptide or genetic sequences may be used as source organisms in accordance with the present invention. Representative source organisms include, for example, animal, mammalian, insect, plant, fungal, yeast, algal, bacterial, archaebacterial, cyanobacterial, and protozoal source organisms. For example a source organism may be a fungus, including yeasts, of the genus Saccharomyces, Yarrowia, Aspergillus, Schizosaccharomyces, or Kluyveromyces. In certain embodiments, the source organism may be of the species Saccharomyces cerevisiae, Yarrowia lipolytica, Aspergillus niger, Aspergillus oryzae, Schizosaccharomyces pombe, or Kluyveromyces lactis. For example a source organism may be a bacterium, including an archaebacterium, of the genus Nocardia, Methanothermobacter, Actinobacillus, Escherichia, Erwinia, (Thermo)synechococcus, Streptococcus or Corynebacterium. In certain embodiments, the source organism may be of the species Nocardia sp. JS614, Methanothermobacter thermautotrophicus str. Delta H, Actinobacillus succinogenes, Actinobacillus pleuropneumoniae, Escherichia coli, Erwinia carotovora, Erwinia chrysanthemi, (Thermo)synechococcus vulcanus, Streptococcus bovis or Corynebacterium glutamicum. For example a source organism may be a plant of the genus Arabidopsis, Brassica or Triticum. In certain embodiments, the source organism may be of the species Arabidopsis thaliana, Brassica napus or Triticum secale. For example a source organism may be a mammal of the genus Rattus, Mus or Homo. In certain embodiments, the source organism may be of the species Rattus norvegicus, Mus musculus or Homo sapiens.

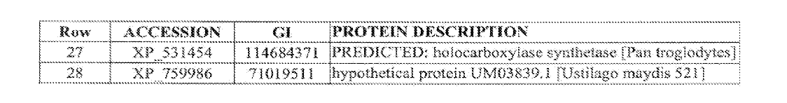

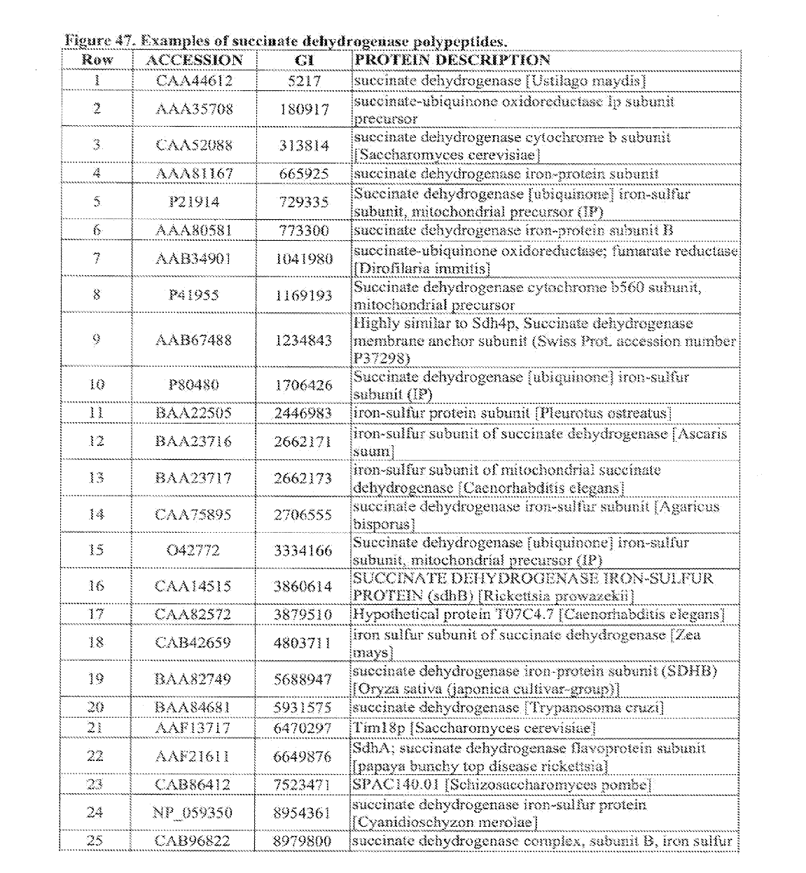

Succinate dehydrogenase (SDH) polypeptides: “Succinate dehydrogenase (SDH) (complex II or succinate:ubiquinone oxidoreductase) polypeptides” are polypeptides that participate in the aerobic mitochondrial electron transport chain and tricarboxylic acid (TCA) cycle by oxidizing succinate to fumarate and transferring the electrons to ubiquinone (EC 1.3.5.1). Two electrons from succinate are transferred one at a time through a flavin cofactor and a chain of iron-sulfur clusters to reduce ubiquinone to an ubisemiquinone intermediate and to ubiquinol. In general, a complex of SDH polypeptides is composed of a catalytic heterodimer and a membrane domain, comprising two smaller hydrophobic subunits that anchor the enzyme to the mitochondrial inner membrane. Succinate dehydrogenase (SDH) of Saccharomyces cerevisiae consists of four subunits encoded by the SDH1, SDH2, SDH3, and SDH4 genes. Examples of succinate dehydrogenase polypeptides are represented by the Genbank GI numbers in FIG. 47 and the succinate dehydrogenase polypeptides in FIG. 29. In some cases, a succinate dehydrogenase polypeptide has an amino acid sequence that is at least about 75%, 85%, 90%, 95% or 100% identical to that of a polypeptide represented by the Genbank GI numbers in FIG. 47 or a succinate dehydrogenase polypeptide in FIG. 29.

Transcription: As is known in the art, the term “transcription” refers to the process of producing an RNA copy from a DNA template.

Transformation: The term “transformation”, as used herein, typically refers to a process of introducing a nucleic acid molecule into a host cell. Transformation typically achieves a genetic modification of the cell. The introduced nucleic acid may integrate into a chromosome of a cell, or may replicate autonomously. A cell that has undergone transformation, or a descendant of such a cell, is “transformed” and is a “recombinant” cell. Recombinant cells are modified cells as described herein. If the nucleic acid that is introduced into the cell comprises a coding region encoding a desired protein, and the desired protein is produced in the transformed yeast and is substantially functional, such a transformed yeast is “functionally transformed.” Cells herein may be transformed with, for example, one or more of a vector, a plasmid or a linear piece (eg., a linear piece of DNA created by linearizing a vector) of DNA to become functionally transformed.

Translation: As is known in the art, the term “translation” refers to the production of protein from messenger RNA.

Yield: The term “yield”, as used herein, refers to the amount of a desired product (e.g., an organic acid) produced (molar or weight/volume) divided by the amount of carbon source (e.g., dextrose) consumed (molar or weight/volume) multiplied by 100.

Unit: The term “unit”, when used to refer to an amount of an enzyme, refers to the enzymatic activity and indicates the amount of micromoles of substrate converted per mg of total cell proteins per minute.

Vector: The term “vector” as used herein refers to a DNA or RNA molecule (such as a plasmid, linear piece of DNA, cosmid, bacteriophage, yeast artificial chromosome, or virus, among others) that carries nucleic acid sequences into a host cell. The vector or a portion of it can be inserted into the genome of the host cell.

Vitamin H transport (VHT) polypeptides: “Vitamin H transport (VHT) polypeptides” are polypeptides that mediate biotin uptake through a carrier-mediated and energy-requiring mechanism. Many fungal species are biotin auxotrophs; VHT polypeptide activity may be essential for growth in such strains. VHT polypeptides are members of a major facilitator superfamily. Examples of VHT polypeptides are represented by the Genbank GI numbers in FIG. 48 and the VHT polypeptides in FIG. 29. In some cases, a VHT polypeptide has an amino acid sequence that is at least about 75%, 85%, 90%, 95% or 100% identical to that of a polypeptide represented by the Genbank GI numbers in FIG. 48 or a VHT polypeptide in FIG. 29.

### DESCRIPTION OF CERTAIN ILLUSTRATIVE EMBODIMENTS

### Organic Acids

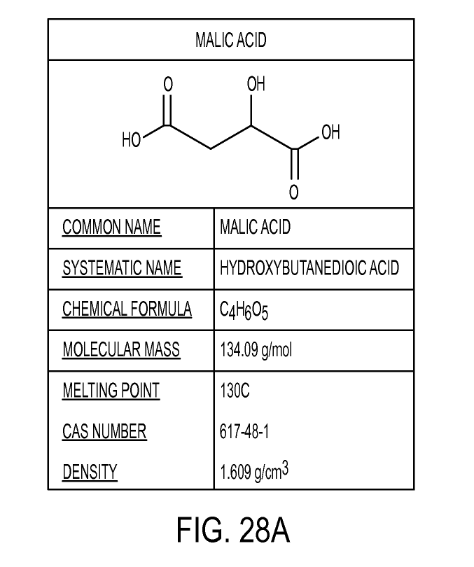

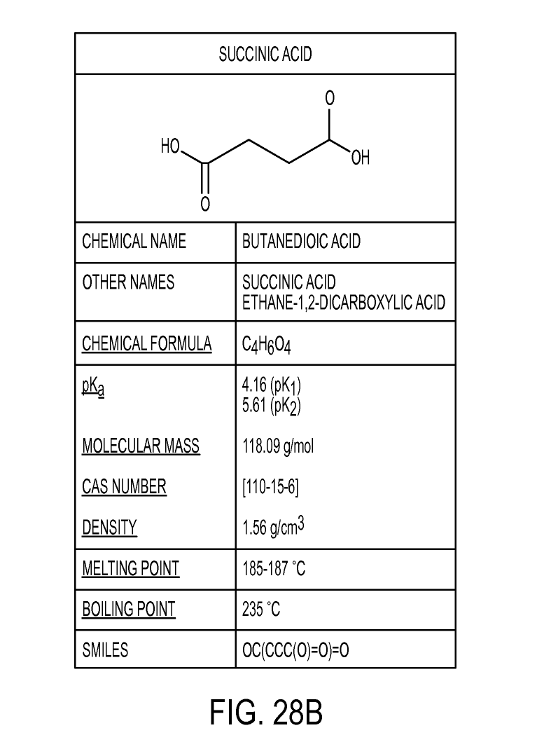

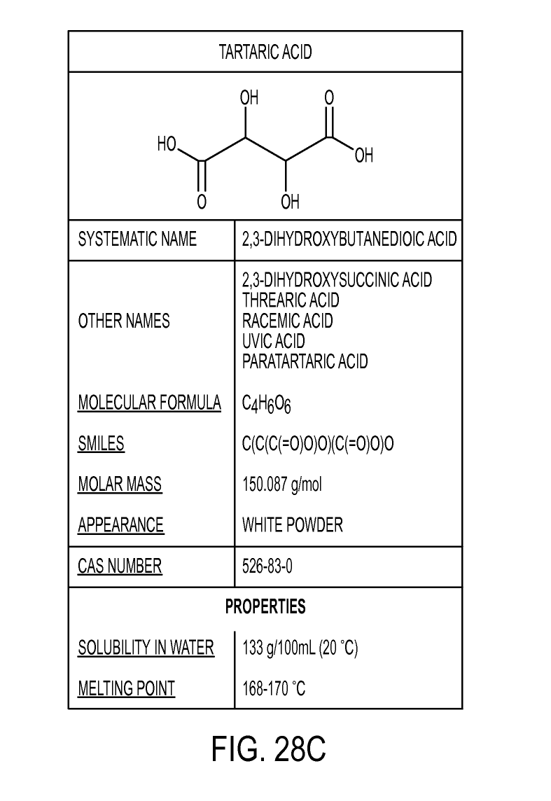

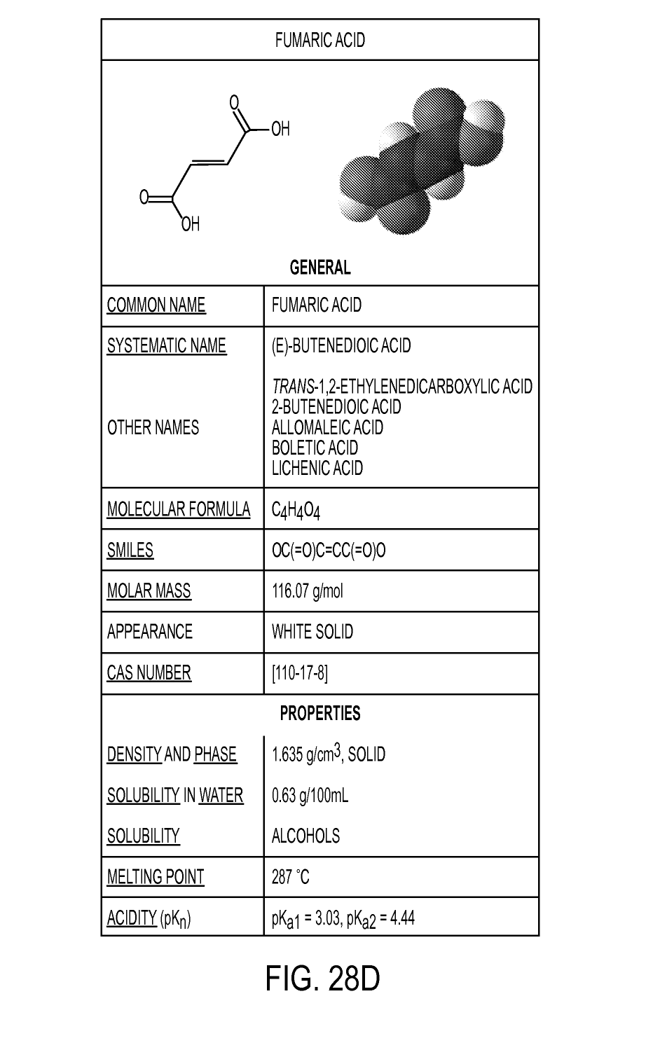

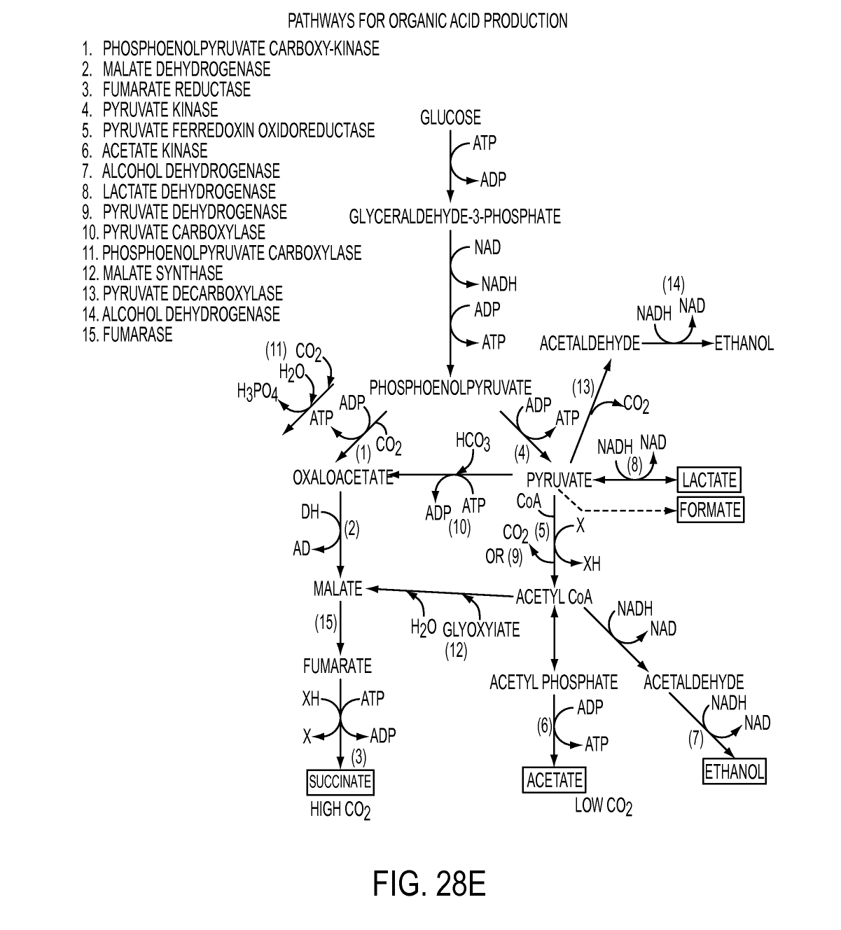

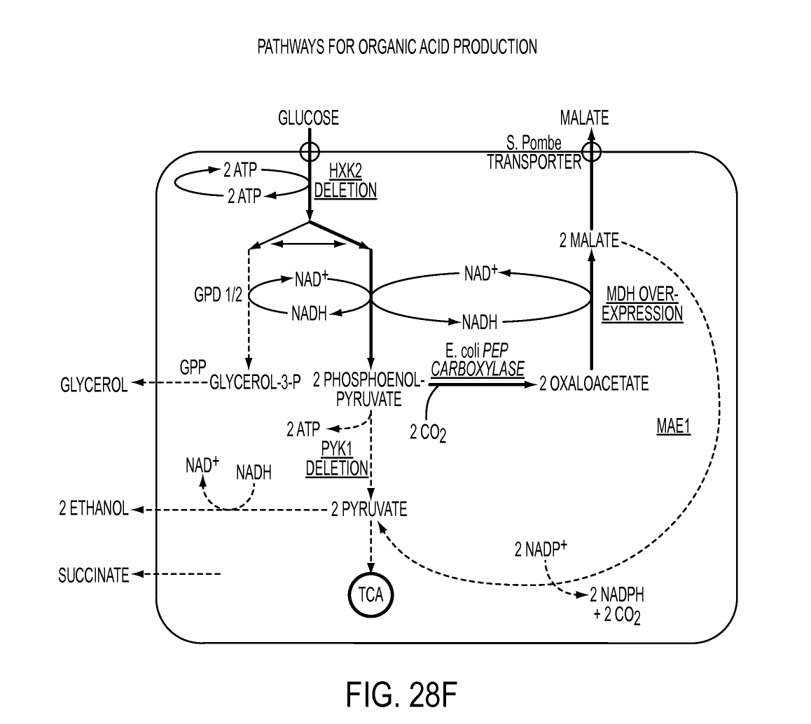

Described herein are methods that can be used to engineer host cells to produce any of a variety of organic acid compounds. In certain cases, for example, the methods are utilized to engineer host cells to produce one or more of fumaric acid, malic acid, succinic acid, and tartaric acid (see FIG. 28a-g). In certain cases the methods are utilized to engineer host cells to produce one or more derivatives of one or more of these compounds.

### Fumaric Acid

Fumaric acid (FIG. 28d) is naturally produced by a variety of organisms including, for example, fumitory (Fumaria officinalis), bolete mushrooms (specifically Boletus fomentarius var. pseudo-igniarius), lichen, and Iceland moss, and is also an intermediate (in the form of fumarate) in the citric acid cycle.

Fumaric acid is non-toxic and is widely used in the food industry, for example as a food acidulant. Fumaric acid can be used as a substitute for tartaric acid and/or citric acid (for which it is commonly substituted at the level of 0.91 g fumaric for each 1.36 g citric), and is a common component of food additives, dietary supplements and candy. Fumaric acid is a useful intermediate in the preparation of a variety of edible products including, for example, malic acid and aspartic acid.

Fumaric acid is used as an industrial chemical in the manufacture of polyester resins and polyhydric alcohols and as a mordant for dyes.

Also, fumaric acid esters are sometimes used to treat psoriasis (e.g., at a dose ranging from approximately 50-60 mg/day to over 1200 mg/day).

In general, fumaric acid can be used in the production of a wide variety of industrial chemicals as fumaric acid derivatives, including but not limited to, tetrahydrofuran (THF), butanediol (e.g. 1,4-butanediol), γ-butyrolactone, pyrrolidinones (e.g. N-methyl-2-pyrrolidone), esters, diamines, 4,4-Bionelle, hydroxybutyric acid, dibasic ester (DBE), maleic anhydride, succindiamide, 1,4-diaminobutane, succinonitrile, unsaturated succinate derivatives, polymers (including biodegradable polymers) such as polybutylene terephthalate. These derivatives may be produced by any number of processes including physical treatments, fermentation, biocatalysis, chemical transformation and combinations thereof.

### Malic Acid

Malic acid (FIG. 28a) is a tart-tasting compound that is naturally produced by many fruits and is also used in the production of a variety of foods. Beneficial traits of malic acid for the food industry include flavor enhancement relative to other products, desirable properties for blending with other ingredients, and chelating abilities to increase the solubility and availability of ions such as calcium. Malic acid is currently used in the production of a wide range of foods, including beverages, confectioneries (particularly sour-tasting candies) and bakery products, as well as food preservatives.

In beverages, malic acid improves flavors and masks the tastes of some salts and sweeteners; it also improves pH stability and provides several desirable properties to calcium fortified drinks. In candies, malic acid provides lingering sourness and exceptional blending properties, including its high solubility at relatively low temperatures. Malic acid functions to provide consistent texture and balanced flavor in bakery products. In food applications, malic acid can also be used in edible and antimicrobial films and coatings, which can also be further treated with a variety of powdered ingredients.

Malic acid is also currently utilized in the cosmetic industry, for example as part of face and/or body lotions, as well as in nail enamel compositions that are made of polymers plasticized with esters of malic acid.

Malic acid is also utilized in the chemical industry, and has significant potential for many high-volume industrial chemical applications derived from a malic acid feedstock. These applications include, for example, surfactants and biodegradable polymers. Particularly useful industrial chemical derivatives of malic acid include, but are not limited to, hydroxybutyrolactone and hydroxysuccinate derivatives, maleic anhydride, 1,4-butanediol, and polymers (including biodegradable polymers) such as polymalic acid and polybutylene terephthalate. These derivatives may be produced by any number of processes including physical treatments, fermentation, biocatalysis, chemical transformation and combinations thereof.

### Succinic Acid

Succinic acid (in the form of its anion, succinate) is naturally produced in a variety of organisms as an intermediate in the citric acid cycle (see FIGS. 28b and e-g); it is also produced by many anaerobic microbes as the major end-product of their energy metabolism. To give but a few examples, succinate is produced from sugars or amino acids by proprionate-producing bacteria such as, for example, species of Propionibacterium, by typical gastrointestibal bacteria such as Anaerobiospirillum succiniciproducens, Bacteroides sp., Escherichia coli, Pectinaturs sp., etc, and by rumen bacteria such as, for example, Actinobacillus succinogens, Bacteroides amylophilus, Cytophaga succinans, Fibrobacter succinogens, Mannheimia succiniciproducens, Prevotella ruminicola, Ruminococcus flavefaciens, Succinimonas amylolytica, Succinivibrio dextrinicolvens, Wolinella succinogenes, etc., as well as by various Lactobacillus strains.

Succinic acid is currently marketed as a surfactant/detergent/extender/foaming agent. Succinic acid is also useful as an ion chelator. For instance, succinic acid is commonly utilized in electroplating in order to reduce corrosion or pitting of metals.

Succinic acid is also utilized in the food industry, for example, as an acidulant/pH modifier, a flavoring agent (.e.g., in the form of sodium succinate), and/or an anti-microbial agent. Succinic acid can also be employed as a feed additive. Succinic acid can be utilized to improve the properties of soy proteins in food or feed through the succinylation of lysine residues.

Succinic acid also finds utility in the pharmaceutical/health products market, for example in the production of pharmaceuticals (including antibiotics), amino acids, vitamins, etc.

Succinic acid can also be utilized as a plant growth stimulant.

Succinic acid, like malic and fumaric acid, further can be employed as an industrial chemical in the commodity and/or specialty chemicals markets, for example as an intermediate in the production of compounds such as adipic acid (e.g., for use as the precursor to nylon and/or in the manufacture of lubricants, foams, and/or food products), 4-amino butanoic acid, aspartic acid, 1,4-butanediol (e.g., for use as a solvent and/or as raw material for production of polybutylene terephthalate resins and/or automotive or electrical parts), diethyl succinate (e.g., for use as a solvent for cleaning metal surfaces or for paint stripping), ethylenediaminedisuccinate (e.g., as a replacement for EDTA), fumaric acid, gamma-butyrolactone (e.g., for use in paint removers and/or textile products, and/or as the raw material for production of pyrrolidone derivatives), hydroxysuccinimide, itaconic acid, maleic acid, maleic anhydride, maleimide, malic acid, N-methylpyrrolidone (e.g., for use as a solvent), 2-pyrrolidione, succinimide, tetrahydrofuran (e.g., for use as a solvent and/or in adhesives, printing inks, magnetic tapes, etc), or other 4-carbon compounds. Succinic acid can also be useful for the production of polymers (including biodegradable polymers) such as polybutylene terephthalate and polybutylene succinate These succinate derivatives may be produced by any number of processes including physical treatments, fermentation, biocatalysis, chemical transformation and combinations thereof.

Succinic acid can also be utilized to modify other compounds and thereby to improve or adjust their properties. For example, succinylation of proteins (e.g., on lysine residues) can improve their physical or functional attributes; succinylation of cellulose can improve water absorbitivity; succinylation of starch can enhance its utility as a thickening agent, etc.

### Tartaric Acid

Tartaric acid (FIG. 28c) is found in the juice of many fruits, and is the source of the “wine diamond” crystals (of potassium bitartrate) that sometimes form on wine corks. Like other organic acids, tartaric acid can play an important role in fruit juice, acting as a preservative due to its ability to inhibit microbial growth through pH reduction. Tartaric acid is included in many foods, especially sour-tasting sweets. As a food additive, tartaric acid is used as an antioxidant or an emulsifier.

Tartaric acid is also utilized as a chelator.

Important derivatives of tartaric acid include its salts, Cream of tartar (potassium bitartrate), Rochelle salt (potassium sodium tartrate, a mild laxative) and tartar emetic (antimony potassium tartrate).

### Host Cells

Any of a variety of host cells may be engineered to increase the production of one or more organic acid compounds. It will often be desirable to utilize cells that are amenable to manipulation, particularly genetic manipulation, as well as to growth on large scale and under a variety of conditions. In certain cases, it will be desirable to utilize host cells that are amenable to growth under anaerobic conditions, microaerobic conditions, and/or under conditions of relatively low pH.

In many cases, it will be desirable to utilize yeast or fungal host cells. Any yeast known in the art for use in industrial processes can be used as a matter of routine experimentation by the skilled artisan having the benefit of the present disclosure. For example, the yeast to be modified (e.g., transformed) can be selected from any known genus and species of yeast. Yeasts are described by N. J. W. Kreger-van Rij, “The Yeasts,” Vol. 1 of Biology of Yeasts, Ch. 2, A. H. Rose and J. S. Harrison, Eds. Academic Press, London, 1987. In one embodiment, the yeast genus can be Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, or Schwanniomyces, among others. In a further embodiment, the yeast can be a Saccharomyces, Zygosaccharomyces, Yarrowia, Kluyveromyces or Pichia spp. In yet a further embodiment, the yeasts can be Saccharomyces cerevisiae, Saccharomyces cerevisiae var bayanus (e.g. Lalvin DV10), Saccharomyces boulardii, Zygosaccharomyces bailii, Kluyveromyces lactis, and Yarrowia lipolytica. Saccharomyces cerevisiae is a commonly used yeast in industrial processes, but the invention is not limited thereto. Other yeast species useful in the present disclosure include but are not limited to Hansenula anomala, Schizosaccharomyces pombe, Candida sphaerica, and Schizosaccharomyces malidevorans.

### Engineering Organic Acid Production

In some cases, a parental cell naturally produces the relevant organic acid compound(s), and is modified to increase production and/or accumulation of the compound(s); in some cases, a parental cell does not naturally produce the relevant organic acid compound(s).

In general, any modification may be applied to a cell to increase or impart production and/or accumulation of one or more desired organic acid compounds. In many cases, the modification comprises a genetic modification. In general, genetic modifications may be introduced into cells by any available means including chemical mutation and/or transfer (e.g., via transformation or mating) of nucleic acids. A nucleic acid to be introduced into a cell according to the present invention may be prepared by any available means. For example, it may be extracted from an organism's nucleic acids or synthesized by chemical means. Nucleic acids to be introduced into a cell may be, but need not be, in the context of a vector.

Genetic modifications that increase activity of a polypeptide include, but are not limited to: introducing one or more copies of a gene encoding the polypeptide (which may differ from any gene already present in the host cell encoding a polypeptide having the same activity); altering a gene present in the cell to increase transcription or translation of the gene (e.g., altering, adding additional sequence to, deleting sequence from, replacement of one or more nucleotides, or swapping for example, a promoter, regulatory or other sequence); and altering the sequence (e.g. coding or non-coding) of a gene encoding the polypeptide to increase activity (e.g., by increasing catalytic activity, reducing feedback inhibition, targeting a specific subcellular location, increasing mRNA stability, increasing protein stability).

Genetic modifications that decrease activity of a polypeptide include, but are not limited to: deleting all or a portion of a gene encoding the polypeptide; inserting a nucleic acid sequence that disrupts a gene encoding the polypeptide; altering a gene present in the cell to decrease transcription or translation of the gene or stability of the mRNA or polypeptide encoded by the gene (for example, by altering, adding additional sequence to, deleting sequence from, replacement of one or more nucleotides, or swapping for example, a promoter, regulatory or other sequence).

A vector for use in accordance with the present methods can be a plasmid, linear piece of DNA, a cosmid, or a yeast artificial chromosome, among others known in the art to be appropriate for use in yeast. A vector can comprise an origin of replication, which allows the vector to be passed on to progeny cells of a parent cell comprising the vector. Alternatively, if integration of the vector into the host cell genome is desired, the vector can comprise sequences that direct such integration (e.g., specific sequences or regions of homology, etc.).

Nucleic acids to be introduced into a cell may be so introduced together with at least one detectable marker (e.g., a screenable or selectable marker). In some embodiments, a single nucleic acid molecule to be introduced may include both a sequence of interest (e.g., a gene encoding a polypeptide of interest as described herein) and a detectable marker. In general, a detectable marker allows cells that have received an introduced nucleic acid to be distinguished from those that have not. For example, a selectable marker may allow transformed cells to survive on a medium comprising an antibiotic fatal to untransformed yeast, or may allow transformed cells to metabolize a component of the medium into a product not produced by untransformed cells, among other phenotypes.

As will be appreciated, nucleic acids can be introduced into cells by any available means including, for example, chemical-mediated transformation, particle bombardment, electroporation, Agrobacterium-mediated transformation, etc.

Nucleic acids to be expressed in a cell are typically in operative association with one or more expression sequences such as, for example, promoters, terminators, and/or other regulatory sequences. Any such regulatory sequences that are active in the host cell (including, for example, homologous or heterologous host sequences, constitutive, inducible, or repressible host sequences, etc.) can be used.

A promoter, as is known, is a DNA sequence that can direct the transcription of a nearby coding region. A promoter can be constitutive, inducible or repressible. Constitutive promoters continually direct the transcription of a nearby coding region. Inducible promoters can be induced by the addition to the medium of an appropriate inducer molecule, which will be determined by the identity of the promoter. Repressible promoters can be repressed by the addition to the medium of an appropriate repressor molecule, which will be determined by the identity of the promoter.

Representative useful promoters include, for example, the constitutive promoter S. cerevisiae triosephosphate isomerase (TPI) promoter, the S. cerevisiae glyceraldehyde-3-phosphate dehydrogenase (isozyme 3) TDH3 promoter, the S. cerevisiae TEF1 promoter and the S. cerevisiae ADH1 promoter. Representative terminators for use in accordance with the present invention include, for example, S. cerevisiae CYC1.

In some cases, a genetic modification is one that involves disruption of one or more nucleic acid sequences present in a cell. Such disruption may be achieved by any desired means including, for example, chemical disruption and/or integration of disrupting nucleic acid sequences, etc.

Alternatively or additionally, a genetic modification may comprise introduction of one or more new nucleic acids into a cell. In some cases, the introduced nucleic acid sequences may be from a heterologous source; in some embodiments, introduced nucleic acid sequences may represent additional or alternative copies of sequences already present in the cell.

In some cases, introduced or disrupted nucleic acid sequences encode one or more anaplerotic polypeptides and/or one or more C4-dicarboxylic acid biosynthetic polypeptides and/or one or more GSR polypeptides and/or one or more hexose transporter polypeptides and/or one or more organic acid transporter (OAT) polypeptides and/or one or more pyruvate decarboxylase (PDC) polypeptides.

In some cases, where nucleic acid sequences originating from a source heterologous to the host cell are utilized, such sequences may be modified, for example, to adjust for codon preferences and/or other expression-related aspects (e.g., linkage to promoters and/or other regulatory sequences active in the host cell, etc.) of the host cell system.

In some cases, polypeptides or nucleic acid sequences introduced into or present within a host cell may contain sequences that alter localization and/or site of activity of a polypeptide, and particularly of a C4-dicarboxylic acid biosynthetic or an OAT polypeptide. In some cases, for example, it may be desirable for polypeptides involved in organic acid production in modified host cells to be present and/or active in the cytosol. Localizing sequences (e.g., sequences that target polypeptides to the cytosol, to and organelle such as the mitochondria, to membranes, for secretion, etc.) may be added to or removed from utilized nucleic acids.

Cells may be modified to produce and/or accumulate one or more organic acids by any biosynthetic route. Representative paths for the production of organic acid such as malic acid, fumaric acid, succinic acid and tartaric acids are shown in FIGS. 28e, 28f and 28g.

In some cases, a cell is modified to increase its anaplerotic acitivity. Alternatively or additionally, a cell may be modified to decrease pyruvate decarboxylase activity, to increase or decrease organic acid transport activity, to increase or decrease glucose sensing and regulatory polypeptide activity, to increase or decrease hexose transporter (HXT) activity, and/or to increase or decrease C4 dicarboxylic acid biosynthetic activity. In some embodiments, a cell contains at least two or more such modifications.

Representative modifications that increase anaplerotic activity include, for example, those that increase pyruvate carboxylase (PYC) activity, those that increase phosphoenolpyruvate carboxylate (PPC) activity, those that increase or decrease phosphoenolpyruvate carboxykinase (PCK) activity, those that increase or decrease pyruvate kinase (PYK) activity, those that increase biotin protein ligase (BPL) activity, those that increase biotin transport protein (VHT) activity, those that increase or decrease bicarbonate transport activity, and/or those that increase carbonic anhydrase activity.

Production and Isolation of Organic Acids

After a modified (e.g., recombinant) yeast has been obtained, the yeast can be cultured in a medium. The medium in which the yeast can be cultured can be any medium known in the art to be suitable for this purpose. Culturing techniques and media are well known in the art. In one embodiment, culturing can be performed by aqueous fermentation in an appropriate vessel. Examples for a typical vessel for yeast fermentation comprise a shake flask or a bioreactor.

The medium can comprise a carbon source such as glucose, sucrose, fructose, lactose, galactose, or hydrolysates of vegetable matter, among others. In some cases, the medium can also comprise a nitrogen source as either an organic or an inorganic molecule. Alternatively or additionally, the medium can comprise components such as amino acids; purines; pyrimidines; corn steep liquor; yeast extract; protein hydrolysates; water-soluble vitamins, such as B complex vitamins; inorganic salts such as chlorides, hydrochlorides, phosphates, or sulfates of Ca, Mg, Na, K, Fe, Ni, Co, Cu, Mn, Mo, or Zn, among others. Further components known to one of ordinary skill in the art to be useful in yeast culturing or fermentation can also be included. The medium can be buffered but need not be. Considerations for selection of medium components include but are not limited to productivity, cost, and impact on the ability to recover desired products (e.g. organic acid(s)).

The carbon dioxide source can be gaseous carbon dioxide (which can be introduced to a headspace over the medium or sparged through the medium) or a carbonate salt (for example, calcium carbonate), for example, incorporated into the medium.

During the course of the fermentation, the carbon source is internalized by the yeast and converted, through a number of steps, into an organic acid (e.g. malic, fumaric, succinic, or tartaric acid). Expression of an OAT polypeptide, including but not limited to MAE polypeptides, allows the organic acid so produced to be secreted by the yeast into the medium.

An exemplary medium is mineral medium containing 50 g/L CaCO₃ and 1 g/L urea.

According to the present invention, a host cell is modified to increase its production of one or more organic acid compounds. The modified fungal cell can be cultured under conditions and for a time sufficient for organic acid (e.g. malic, fumaric, succinic, or tartaric acid) to accumulate. In some cases, such modification increases production of the relevant compound at least about 1.1-fold, at least about 2-fold, at least about 5-fold, at least about 10-fold, at least about 20-fold, at least about 35-fold, at least about 50-fold as compared with an otherwise identical host cell lacking the modification.

According to the present invention, a modified fungal cell can be cultured under conditions and for a time sufficient for organic acid (e.g. malic, fumaric, succinic, or tartaric acid) to accumulate. For example, the organic acid may accumulate to about 0.3 moles of organic acid/moles of substrate, about 0.35 moles of organic acid/moles of substrate, about 0.4 moles of organic acid/moles of substrate, about 0.45 moles of malic organic acid/moles of substrate, 0.5 moles of organic acid/moles of substrate, about 0.55 moles of organic acid/moles of substrate, about 0.6 moles of organic acid/moles of substrate, about 0.65 moles of organic acid/moles of substrate, about 0.7 moles of organic acid/moles of substrate, about 0.75 moles of organic acid/moles of substrate, about 0.8 moles of organic acid/moles of substrate, about 0.85 moles of organic acid/moles of substrate, about 0.9 moles of organic acid/moles of substrate, about 0.95 moles of organic acid/moles of substrate, about 1 moles of organic acid/moles of substrate, about 1.05 moles of organic acid/moles of substrate, about 1.1 moles of organic acid/moles of substrate, about 1.15 moles of organic acid/moles of substrate, about 1.2 moles of organic acid/moles of substrate, about 1.25 moles of organic acid/moles of substrate, about 1.3 moles of organic acid/moles of substrate, about 1.35 moles of organic acid/moles of substrate, about 1.4 moles of organic acid/moles of substrate, about 1.45 moles of organic acid/moles of substrate, about 1.5 moles of organic acid/moles of substrate, about 1.55 moles of organic acid/moles of substrate, about 1.6 moles of organic acid/moles of substrate, about 1.65 moles of organic acid/moles of substrate, about 1.7 moles of organic acid/moles of substrate, about 1.75 moles of organic acid/moles of substrate. In some embodiments, the organic acid accumulates in the medium. In some embodiments the substrate is glucose.

We have observed that culturing a recombinant yeast in mineral medium comprising 50 g/L CaCO₃ and 1 g/L urea can lead to levels of organic acid (as acid) in the medium of at least 1 g/L. In some cases, it can lead to levels of organic acid (as acid) in the medium of at least 10 g/L. In other cases, it can lead to levels of organic acid (as acid) in the medium of at least 30 g/L.

Thus, in certain cases, the organic acid (e.g. malic, fumaric, succinic, or tartaric acid) accumulates in the medium to at least about 20 g/L, at least about 30 g/L, at least about 40 g/L, at least about 50 g/L, at least about 60 g/L, at least about 70 g/L, at least about 80 g/L, at least about 90 g/L, at least about 100 g/L, at least about 110 g/L, at least about 120 g/L, at least about 130 g/L, at least about 140 g/L, at least about 150 g/L, at least about 160 g/L, at least about 170 g/L, at least about 180 g/L, at least about 190 g/L, at least about 200 g/L.

Modified yeast can be cultured under conditions where the acidic pH of the medium promotes the accumulation of soluble free organic acid (e.g. malic, fumaric, succinic, or tartaric acid) as the major product form, thereby decreasing economic and environmental costs that result from the need to remove impurities or by-products such as calcium sulfate (gypsum). Thus, in certain cases, the organic acid accumulates in a medium of a pH of at least less than 5.0, at least less than 4.5, at least less than 4.0, at least less than 3.5, at least less than 3.0, at least less than 2.5.

After culturing has progressed for a sufficient length of time to produce a desired concentration of organic acid (e.g., malic, fumaric, succinic, or tartaric acid), the organic acid can be isolated. Specifically, the organic acid can be brought to a state of greater purity by separation of the organic acid from at least one other component (either another organic acid or a compound not in that category) of the yeast or the medium. In some cases, the organic acid is at least about 50%, 55%, 60%, 65%, 70%, 75%, 80%, 85%, 90%, 95%, 96%, 97%, 98%, 99%, pure or more. In some cases, the isolated organic acid is at least about 95% pure, such as at least about 99% pure.

Any available technique can be utilized to isolate the accumulated organic acid (e.g., malic, fumaric, succinic, or tartaric acid). For example, the isolation can comprise purifying the organic acid from the medium by known techniques, such as the use of an ion exchange resin, activated carbon, microfiltration, ultrafiltration, nanofiltration, liquid-liquid extraction, crystallization, or chromatography, among others. Liquid-liquid extraction is a preferred method for recovering protonated carboxylic acids such as malic and/or succinic acid from an aqueous medium. Liquid-liquid extraction is generally performed using a reactive long-chain aliphatic tertiary amine (e.g. triisooctylamine or tridodecylamine) in an extractant containing a modifier (e.g. n-octanol), which enhances the extracting power of the reactive amine, and an inert diluent (e.g. n-heptane). Other organic acid recovery and extraction methods include those disclosed in U.S. Pat. No. 4,670,155, U.S. Pat. No. 5,143,833, U.S. Pat. No. 5,168,055, U.S. Pat. No. 5,034,105, U.S. Pat. No. 5,426,220, U.S. Pat. No. 5,104,492, U.S. Pat. No. 5,510,526, U.S. Pat. No. 5,780,276, U.S. Pat. No. 5,773,653, U.S. Pat. No. 5,412,126, Hanson J (1971) Recent advances in liquid-liquid extraction. Pergmon, Oxford, and Eggeman and Verser (2005) Appl Biochem Biotechnol. 121-124:605-18 including the references cited therein.

### EXEMPLIFICATION

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Two yeast strains were constructed starting with S. cerevisiae strain TAM (MATa pdc1(-6,-2)::loxP pdc5(-6,-2)::loxP pdc6(-6,-2)::loxP ura3-52 (PDC-negative)), which was transformed with genes encoding a pyruvate carboxylase (PYC), a malate dehydrogenase (MDH), and a malate transporter protein (MAE).

Because the TAM strain has only one auxotrophic marker, we disrupted the TRP1 locus in order to be able to introduced more than one plasmid with an auxotrophic marker, resulting in RWB961 (MATa pdc1(-6,-2)::loxP pdc5(-6,-2)::loxP pdc6(-6,-2)::loxP mutx ura3-52 trp1::Kanlox).

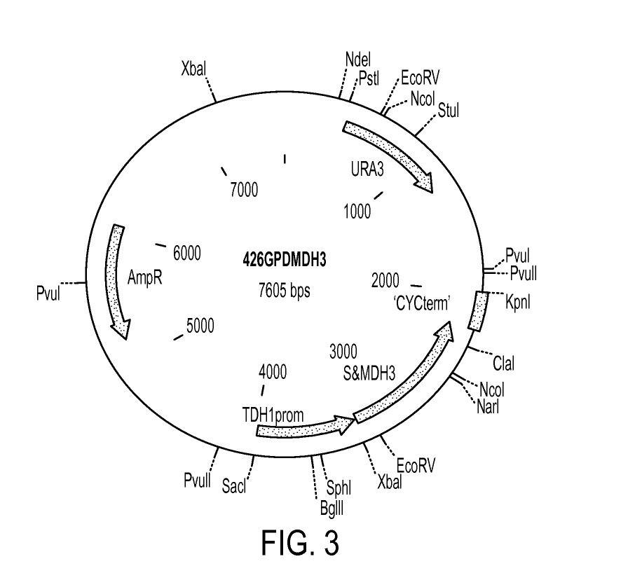

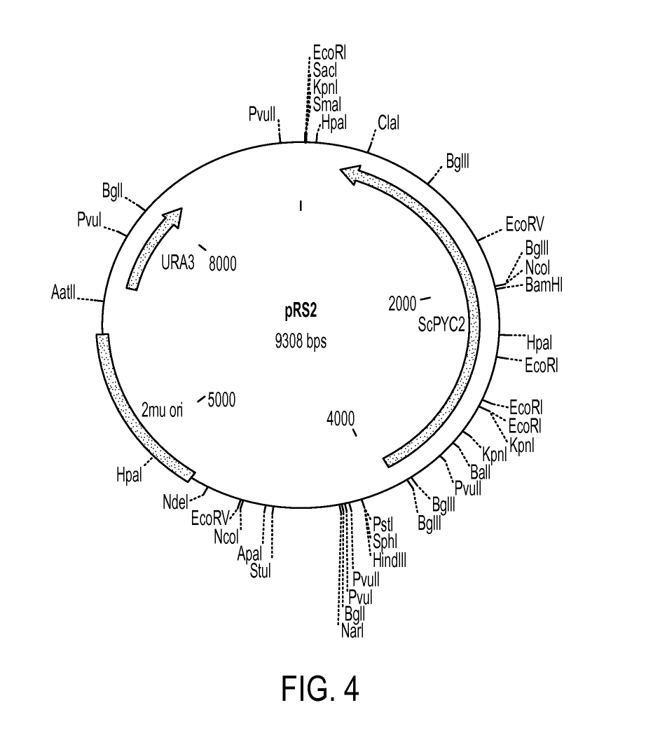

The MDH and PYC genes we used had been previously cloned into plasmids p426GPDMDH3 (2μ plasmid with URA3 marker, containing the MDH3ΔSKL gene between the S. cerevisiae THD3 promoter and the S. cerevisiae CYC1 terminator, FIG. 3) and pRS2 (2μ plasmid with URA3 marker containing the S. cerevisiae PYC2 gene, FIG. 4).

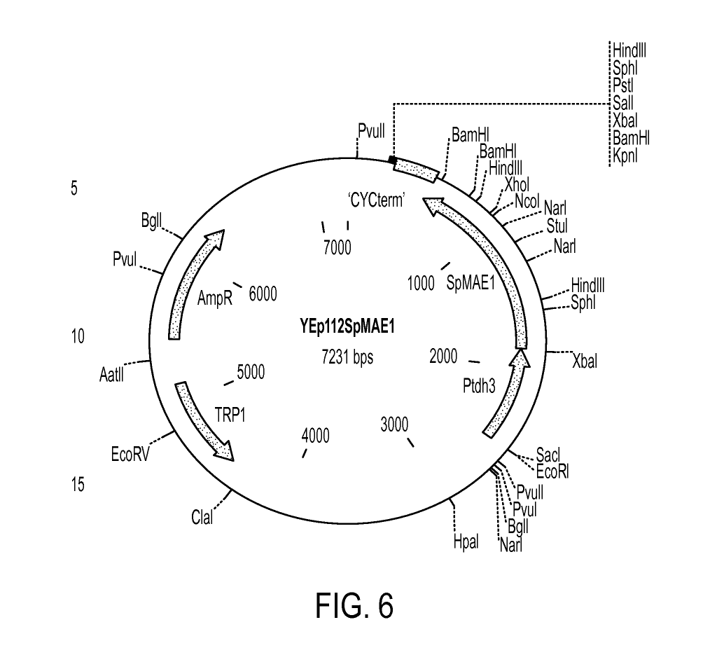

A P_{TDH3}-SpMAE1 cassette carrying the S. pombe MAE was recloned into YEplac112 (2μ, TRP1) and YIplac204 (integration, TRP1), resulting in YEplac112SpMAE1 (FIG. 6) and YIplac204SpMAE1 (not shown).

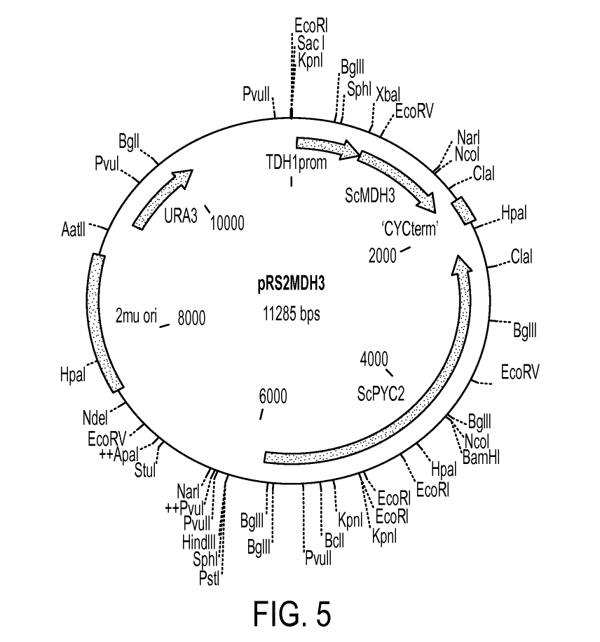

A PYC and MDH vector was prepared: pRS2MDH3ΔSKL (2μ, URA3, PYC2, MDH3ΔSKL) (FIG. 5).

RWB961 was transformed with pRS2MDH3ΔSKL and YEplac112SpMAE1 (strain 1) or pRS2MDH3ΔSKL and YIplac204SpMAE1 (strain 2). Both strain 1 and strain 2 overexpressed PYC2 and MDH3ΔSKL, but had different levels of expression for the MAE1, assuming expression levels were proportional to plasmid copy number, about 10-40 per cell for YEplac112SpMAE1 (2μ-based) and about 1-2 per cell for YIplac204SpMAE1 (integrated).

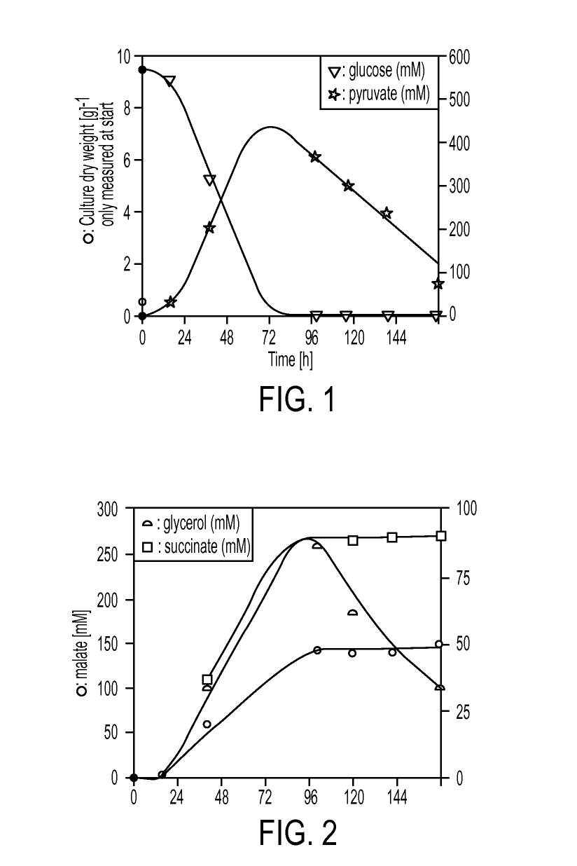

After isolation of strain 1 and strain 2, 0.04 g/L or 0.4 g/L of each strain was introduced to a 500 mL shake flask containing 100 mL mineral medium, 50 g/L CaCO₃, and 1 g/L urea. Flasks were shaken at 200 rpm for the duration of each experiment. Samples of each culture medium were isolated at various times and the concentrations of glucose, pyruvate, glycerol, succinate, and malate determined. Extracellular malate concentrations of about 250 mM after about 90-160 hr were observed. Results are shown in FIGS. 1-2.

The results indicate that the following modifications to yeast metabolic pathways allow high levels of extracellular malate accumulation by recombinant yeasts:

1. Direct the pyruvate flux towards pyruvate carboxylase (by reducing PDC activity).

2. Increase flux through pyruvate carboxylase by overexpressing PYC.

3. Introduce high malate dehydrogenase activities in the cytosol to capture oxaloacetate formed by PYC.

4. Introduce a heterologous malic acid transporter to facilitate export of malate. FIG. 2 also shows that extracellular succinate concentrations of about 50 mM could be produced simultaneously with the malate production described above.

### Example 2

The effect of carbon dioxide on malate production in a fermenter system was studied using a TAM strain overexpressing PYC2, cytosolic MDH3, and a S. pombe MAE1 transporter (YEplac112SpMAE1), as described in Example 1. Three fermenter experiments were performed:

A: Batch cultivations under fully aerobic conditions.

B: Batch cultivations under fully aerobic conditions with a mixture of N₂/O₂/CO₂ of 70%/20%/10%.

C: Batch cultivations under fully aerobic conditions with a mixture of N₂/O₂/CO₂ of 65%/20%/15%

Protocol

Media

The mineral medium contained 100 g glucose, 3 g KH₂PO₄, 0.5 g MgSO₂.7H₂O and 1 ml trace element solution according to Verduyn et al (Yeast 8: 501-517, 1992) per liter of demineralized water. After heat sterilization of the medium 20 min at 110° C., 1 ml filter sterilized vitamins according to Verduyn et al (Yeast 8: 501-517, 1992) and a solution containing 1 g urea were added per liter. Addition of 0.2 ml per liter antifoam (BDH) was also performed. No CaCO₃ was added.

Fermenter Cultivations

The fermenter cultivations were carried out in bioreactors with a working volume of 1 liter (Applikon Dependable Instruments, Schiedam, The Netherlands). The pH was automatically controlled at pH 5.0 by titration with 2 M potassium hydroxide. The temperature, maintained at 30° C., is measured with a Pt100-sensor and controlled by means of circulating water through a heating finger. The stirrer speed, using two rushton impellers, was kept constant at 800 rpm. For aerobic conditions, an air flow of 0.5 l·min⁻¹ was maintained, using a Brooks 5876 mass-flow controller (Brooks BV, Veenendaal, The Netherlands), to keep the dissolved-oxygen concentration above 60% of air saturation at atmospheric pressure.

In batches B and C, increased carbon dioxide concentration of 10% or 15% while maintaining a good oxygenation was reached by mixing pressurized air 79% N₂+21% O₂ and a gas mixture containing 79% CO₂+21% O₂ (Hoekloos, Schiedam, the Netherlands). The desired percentage of 10% or 15% CO₂, supplied via a Brooks mass-flow controller, was topped up with pressurized air to a fixed total flow rate of 0.5 L/min.

The pH, DOT and KOH/H2SO4 feeds were monitored continuously using an on-line data acquisition & control system (MFCS/Win, Sartorius BBI Systems).

Off-Gas Analysis

The exhaust gas of the fermenter cultivations was cooled in a condenser (2° C.) and dried with a Perma Pure dryer (type PD-625-12P). Oxygen and carbon dioxide concentrations were determined with a Rosemount NGA 2000 gas analyser. The exhaust gas flow rate was measured with a Saga Digital Flow meter (Ion Science, Cambridge). Specific rates of carbon dioxide production and oxygen consumption were calculated as described by van Urk et al (1988, Yeast 8: 501-517).

Sample Preparation

Samples for biomass, substrate and product analysis were collected on ice. Samples of the fermentation broth and cell free samples (prepared by centrifugation at 10.000×g for 10 minutes) were stored at −20° C. for later analysis.

Determinations of Metabolites

HPLC-Determinations

Determination of sugars, organic acids and polyols were determined simultaneously using a Waters HPLC 2690 system equipped with an HPX-87H Aminex ion exclusion column (300×7.8 mm, BioRad) (60° C., 0.6 ml/min 5 mM H2504) coupled to a Waters 2487 UV detector and a Waters 2410 refractive index detector.

Enzymatic Metabolite Determinations

In order to verify the HPLC measurements and/or exclude separation errors, L-malic acid was determined with an enzymatic kit (Boehringer-Mannheim, Catalog No. 0 139 068).

Determination of Dry Weight

The dry weight of yeast in the cultures was determined by filtering 5 ml of a culture on a 0.45 μm filter (Gelman Sciences). When necessary, the sample was diluted to a final concentration between 5 and 10 g·l⁻¹. The filters were kept in an 80° C. incubator for at least 24 hours prior to use in order to determine their dry weight before use. The yeast cells in the sample were retained on the filter and washed with 10 ml of demineralized water. The filter with the cells was then dried in a microwave oven (Amana Raderrange, 1500 Watt) for 20 minutes at 50% capacity. The dried filter with the cells was weighed after cooling for 2 minutes. The dry weight was calculated by subtracting the weight of the filter from the weight of the filter with cells.

Determination of Optical Density (OD₆₆₀)

The optical density of the yeast cultures was determined at 660 nm with a spectrophotometer; Novaspec II (Amersham Pharmasia Biotech, Buckinghamshire, UK) in 4 ml cuvets. When necessary the samples were diluted to yield an optical density between 0.1 and 0.3.

Batch A: fully aerated 21% O₂ (+79% N₂)

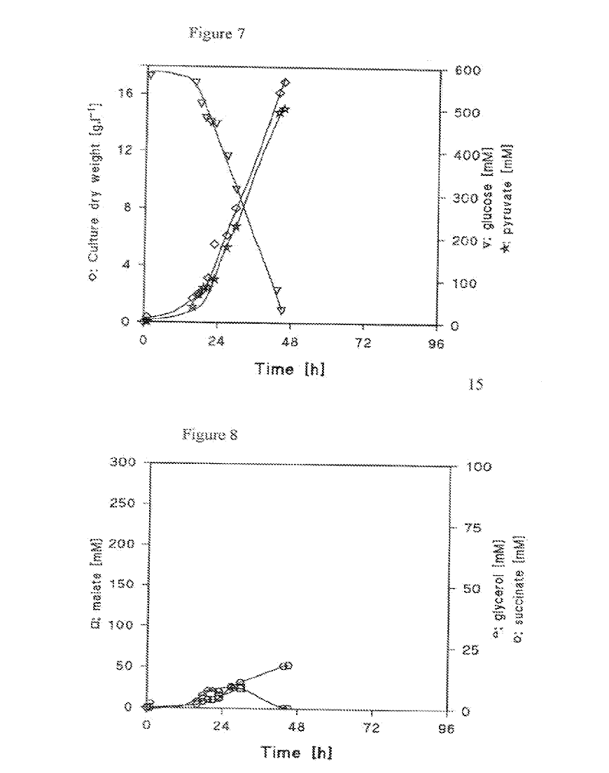

FIGS. 7 and 8 show metabolite formation against time. The result of one representative batch experiment per strain is shown. Replicate experiments yielded essentially the same results. FIG. 7 denotes the biomass (rectangle), the consumption of glucose (triangle) and the production of pyruvate (star). FIG. 8 denotes production of malate (square), glycerol (upper semi circle), and succinate (octagon). As shown in FIG. 8, the yeast produced about 25 mM malate after 24 hr and about 20 mM succinate after 48 hr.

Batch B: 10% CO₂+21% O₂ (+69% N₂)

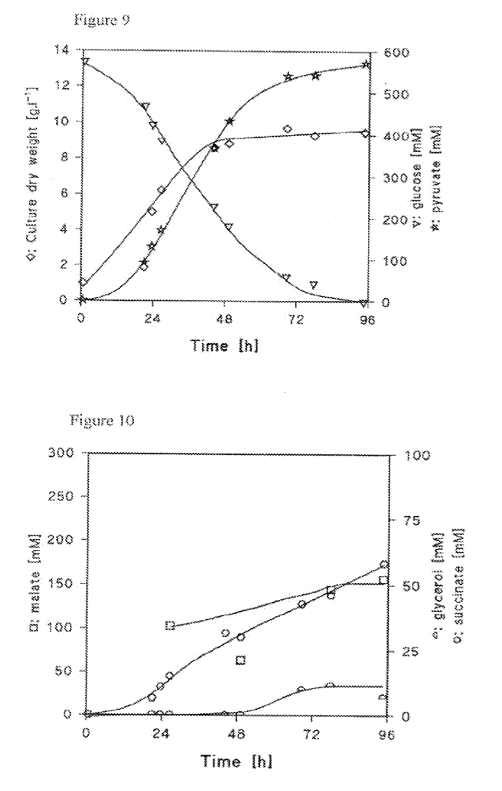

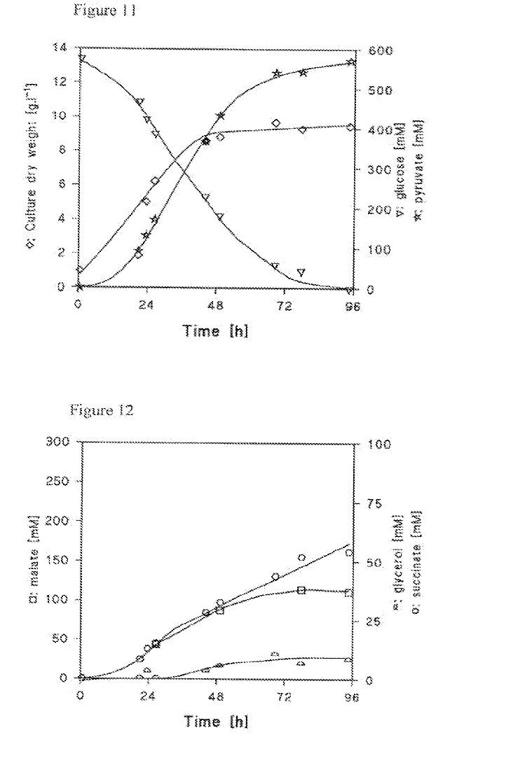

FIGS. 9 and 10 show metabolite formation against time. FIG. 9 denotes the biomass (rectangle), the consumption of glucose (triangle) and the production of pyruvate (star). FIG. 10 denotes production of malate (square), glycerol (upper semi circle), and succinate (octagon). As shown in FIG. 10, the yeast produced about 100 mM malate after 24 hr and about 150 mM malate after 96 hr, as well as about 60 mM succinate after 96 hr.

Batch C: 15% CO₂+21% O₂ (+64% N₂)

FIGS. 11 and 12 show metabolite formation against time. FIG. 11 denotes the biomass (rectangle), the consumption of glucose (triangle) and the production of pyruvate (star). FIG. 12 denotes production of malate (square), glycerol (upper semi circle), and succinate (octagon). As shown in FIG. 10, the yeast produced about 45 mM malate after 24 hr and about 100 mM malate after 96 hr, as well as about 60 mM succinate after 96 hr.

### Example 3

### Disruption of the PYK1 Gene

Multiple strategies exist to carboxylate C3-glycolytic pathway intermediates (e.g PEP and pyruvate) to form the oxaloacetate necessary for organic acid (e.g. fumaric acid, malic acid, succinic acid, and tartaric acid) production. As described above, one strategy exploits the overexpression of PYC polypeptides in a strain that has been modified to prevent carbon flow towards ethanol. In the following examples, it is shown how an alternative approach redirects carbon flow from glycolysis to anaplerosis through the increased activity of PPC polypeptides. Preferred aspects of this strategy involve the dampening of the glycolytic flux to pyruvate by manipulations to decrease the activity of pyruvate kinase; these modifications increase the availability of PEP substrate for PPC polypeptides. In a related strategy described in example 19 herein, phosphoenolpyruvate carboxykinase (PCK) polypeptides can be used to catalyze the carboxylation of PEP. Furthermore, it is possible that aspects of more than one of these strategies can be applied in a single strain in order to optimize flux to oxaloacetate and useful C4-dicarboxylic acids such as malic acid, fumaric acid, tartaric acid and succinic acid.

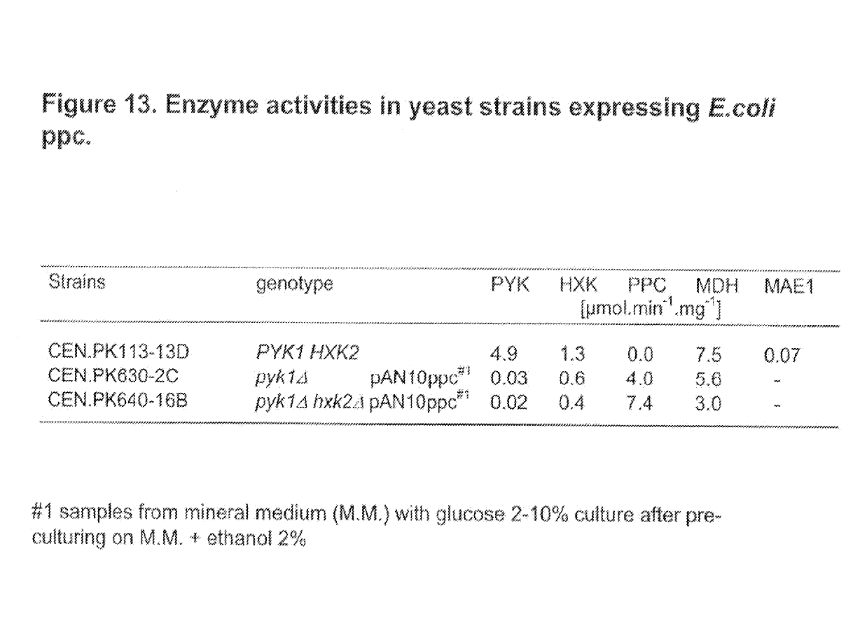

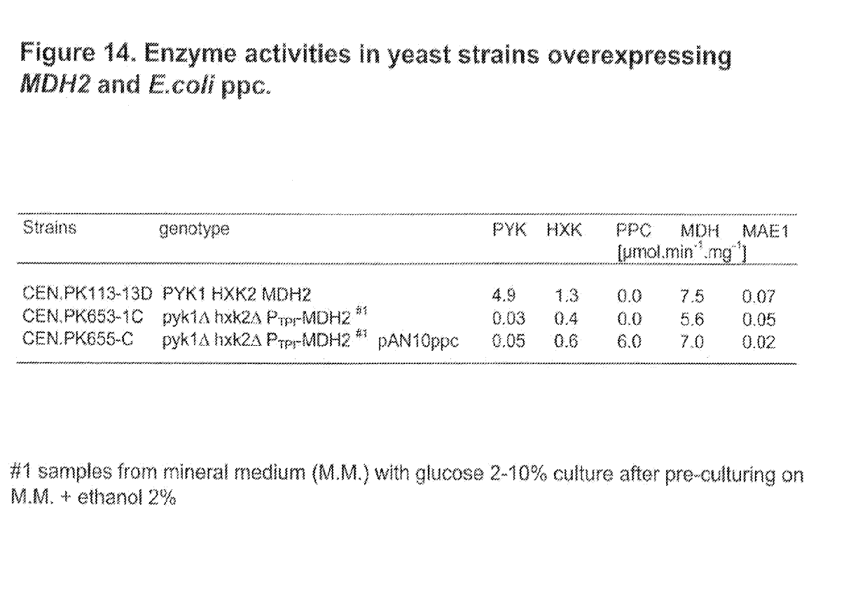

The disruption of the PYK1 gene is advantageous in a strategy that employs PEP carboxylase (PPC) to generate OAA. Disruption of PYK1 eliminates competition between the introduced PPC and the pyruvate kinase (Pyk) in yeast for PEP. Yeast has two genes coding for pyruvate kinase, PYK1 and PYK2. The product of the PYK1 gene accounts for the majority of the pyruvate kinase activity. The PYK1 gene was deleted in a CEN.PK genetic background, and the pyk1 disruption strains, which still contained the PYK2 gene, showed significantly lower pyruvate kinase activities compared to wildtype, 0.05 versus 5 μmol·min⁻¹·mg·protein⁻¹ (see FIGS. 13 and 14). Alternatively strategies for reducing Pyk1 activity have been employed, including the replacement of the wild-type PYK1 gene with the temperature-sensitive alleles of PYK1/CDC19 (see Kaback D B, et al (1984) Genetics 108 (1): 67-90 (1984)).

Physiological analysis showed that the strains with the pyk1 disruption no longer produced ethanol, and the strains did not grow on glucose and had to be pre-cultured on ethanol. Introduction of the pAN10ppc plasmid (2μ plasmid, URA3, E. coli ppc (PEP carboxylase) overproduction) in these strains resulted in biomass production on glucose with a growth rate of approximately 0.007 h⁻¹.

Serial transfer selection experiments with the pyk1 disruption strains on glucose, aimed at an increased growth rate and higher malate production, resulted in strains which again produced ethanol (data not shown). This was ascribed to up regulation of the PYK2 gene, thereby compensating for the pyk1 deletion.

### Example 4

### Preparation of Cell-Free Extracts for Enzyme Determinations

The enzyme samples were obtained from cells growing in chemostat or from shake flasks. When the sample was obtained from shake flask for cells that did not grow on glucose these were first pre-grown on mineral medium with ethanol after which they were transferred to mineral medium with glucose. For preparation of cell extracts, 62.5 mg of biomass were harvested by centrifugation (5 min at 5000 rpm), washed once and re-suspended in 5 ml freeze buffer (10 mM potassium phosphate buffer (pH 7.5) containing 2 mM EDTA). These samples were stored at −20° C. Before preparation of cell extracts, samples were thawed, washed once and re-suspended in 4 ml sonication buffer (100 mM potassium-phosphate buffer (pH 7.5) containing 2 mM MgCl₂ and 1 mM dithiothreitol). Prior to sonication, a teaspoon of glass beads (425-600 μm diameter) was added. Extracts were prepared by sonication in a Sanyo Soniprep 150-sonicator using a 7-8 μm peak-to-peak amplitude for 4 min at 0.5 min intervals. Unbroken cells and debris were removed by centrifugation at 4° C. (20 min at 36,000×g). The clear supernatant was used as the cell extract (For the analysis of malic enzyme the preparation of cell extract was done in 50 mM Tris-HCl buffer pH 7.5 containing 1 mM DTT and 2 mM MgCl2. Cell extracts were dialyzed for 4 hrs at 0° C. against 50 mM Tris buffer by using 0.5-3.0 ml Slyde-a-lyzer cassettes (10,000 MW cutoff, Pierce) (P. de Jong-Gubbels et al. 1998 J. Bact. 180:2875-2882 Identification and characterization of MAE1, the Saccharomyces cerevisiae structural gene encoding mitochondrial malic enzyme).

### Example 5

### Enzyme Assays

All enzyme activities were coupled to (dis)appearance of NAD(P)H or acetyl CoA (acetyl CoA measured via DTNB (5,5-dithiobis-(2-nitrobenzoic acid)), which was monitored spectrophotometrically at 340 nm (ε=6.3 l·mM⁻¹·cm⁻¹) or 412 nm (ε=13.6 l·mM⁻¹·cm⁻¹) respectively. Specific activity of the enzymes was calculated after protein determination via the Lowry method. All enzymes are expressed as Units ((mg) protein)⁻¹. One unit equals 1 μmol of substrate converted per minute under the reaction conditions of the assay. Concentrations are given as the final concentration of each component in the reaction mixture (1 ml in a glass cuvette). In all cases, the reaction rates were checked to be linearly proportional to the amount of cell extract added to the assay.

PYK1—Pyruvate kinase (EC 2.7.1.40):

Cacodylate (pH 6.2) 100 mM, KCl 100 mM, 10 mM, Fructose 1,6-bisphosphate 1 mM, MgCl₂ 25 mM, NADH 0.15 mM, L-Lactate dehydrogenase 11.25 U. Start reaction with: Phosphoenolpyruvate (2 mM).

### MDH2—Malate-dehydrogenase (EC 1.1.1.37):

Potassium phospate buffer (pH 8.0) 100 mM, NADH or NADPH 0.15 mM. Start reaction with oxaloacetate (1 mM).

HXK2—Hexokinase (EC 2.7.1.1):

Imidazole-HCl (pH 7.6) 50 mM, NADP+ 1 mM, MgCl₂ 10 mM, Glucose 10 mM, Glucose-6-P dehydrogenase 1.8 U. Start reaction with ATP (1 mM).

PPC—E. coli PEP carboxylase-pyruvate carboxylase (4.1.1.32):

Imidazole-HCl (pH 6.6) 100 mM, NaHCO₃ 50 mM, MgCl₂ 2 mM, Glutathione 2 mM, ADP 2.5 mM, NADH 2.5 mM, MDH 3 U. Start reaction with: Phosphoenolpyruvate (2.5 mM).
PPC—E. coli PEP carboxylase-pyruvate carboxylase (4.1.1.32):

(alternative assay based on Acetyl-CoA)

Tris-HCl (pH 7.5) 100 M, MgSO₄ 10 mM, KHCO₃ 10 mM, AcCoA 20 mM, KHCO₃ 10 mM, DTNB (5,5-dithiobis-(2-nitrobenzoic acid))/Tris 0.1 mM, citrate synthetase. Start reaction with PEP (5 mM)

Pyruvate carboxylase (4.1.1.32):

(alternative assay based on Acetyl-CoA)

Tris-SO₄ (pH 7.5) 100 M, MgSO₄ 7.5 mM, KHCO₃ 20 mM, AcCoA 0.1 mM, KHCO₃ 20 mM, DTNB (5,5-dithiobis-(2-nitrobenzoic acid))/Tris 0.1 mM, ATP 0.4 mM, Citrate synthetase. Start reaction with K-pyruvate (10 mM).

MAE1—Malic enzyme (EC 1.1.1.40):

Tris-HCl (pH 7.5) 0.1 M, NADP+ 0.4 mM, MgCl₂ 10 mM. Start reaction with L-Malate (100 mM).

### Example 6

### Disruption of the HXK2 Gene

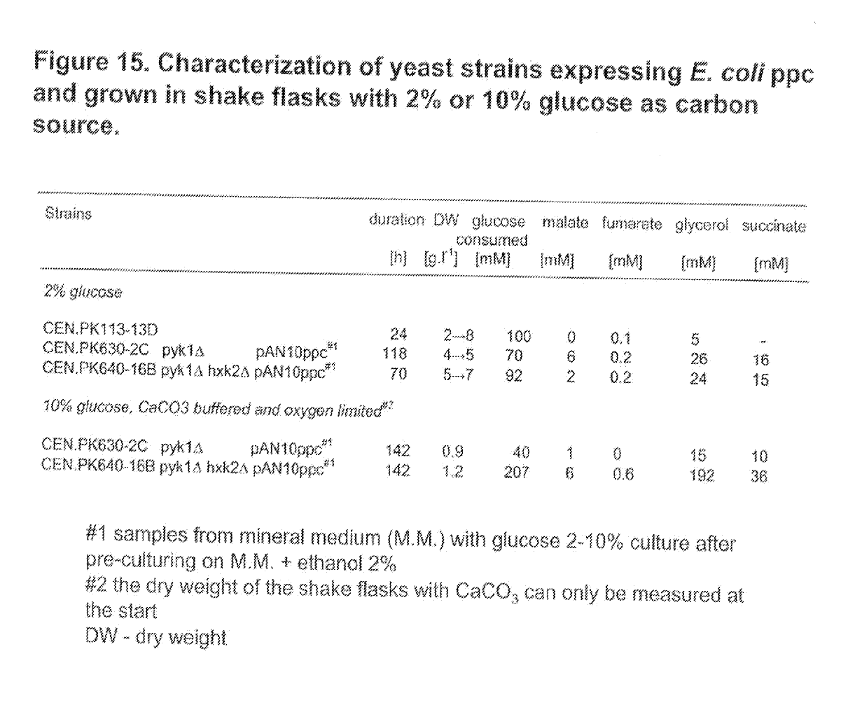

High glucose and sucrose concentrations result in repression of respiratory enzymes, thus limiting the malate production. Disruption of the HXK2 gene has been shown to result in a complete derepression of the respiratory enzymes in the presence of high glucose concentrations (Diderich et al (2001) Appl. Environ. Microbiol. 67:1587-1593; Raamsdonk et al (2001) Yeast 18:1023-1033). Strains were generated that contained hxk2 deletions. Strains with the hxk2 deletion exhibited a lower hexokinase activity than the wild-type strains, respectively 0.5 versus 1.2 μmol·min⁻¹·mg·protein⁻¹ (see FIGS. 13 and 14). In media containing 2% glucose, the rate of glucose consumption in hxk2 pyk1 deleted strains was higher than the strains still containing HXK2. At glucose concentrations of 10%, however, strains containing hxk2 and pyk1 disruptions did not consume glucose (see FIG. 15).

### Example 7

### Overproduction of E. coli PPC

Overproduction of E. coli PEP carboxylase was achieved using the pAN10ppc plasmid (Flores and Gancedo (1997) FEBS Lett. 412: 531-534), containing E. coli ppc gene behind the S. cerevisiae ADH1 promoter. The average in vitro PEP carboxylase activity varied from 4.0-7.4 μmol·min⁻¹·mg·protein⁻¹, depending on the strain and the shake flask conditions (see FIG. 13). The cultivation of the strains containing the pAN10ppc construct (see FIG. 13) on mineral medium with 2% glucose yielded mainly glycerol, carbon dioxide and succinate (see FIG. 15) in comparable yields. The hxk2 disrupted strain consumed the glucose in 70 hours, versus 118 hours for the strain with HXK2.

The E. coli PEP carboxylase was active in vitro but there was no significant malate production. Some malate production was expected since Bauer et al ((1999) FEMS Microbiol. Lett. 179:107-113) reported intracellular malate production when over-expressing native pyruvate carboxylase and thus producing cytosolic oxaloacetate. The main products of the constructed strains consisted of glycerol, carbon-dioxide and succinate. In shake flask conditions on 10% glucose and with oxygen limitation glycerol yields up to 1 mol glycerol per mol of glucose were observed.

### Example 8

### Overexpression of MDH2

Initial attempts to increase cytosolic malate dehydrogenase activity utilized strains that had the TPI1 promoter (P_{TPI}) integrated in front of the chromosomal MDH2 gene, thereby replacing its own promoter (obtained from Peter Kotter (Department of Microbiology Wolfgang Goethe Universitat, Frankfurt). The two strains, CEN.PK653-1C and 655-C, were also both disrupted for pyk1 and hxk2. The strains differed in that one had PPC overexpression (pAN10ppc) and the other did not (see FIG. 14).

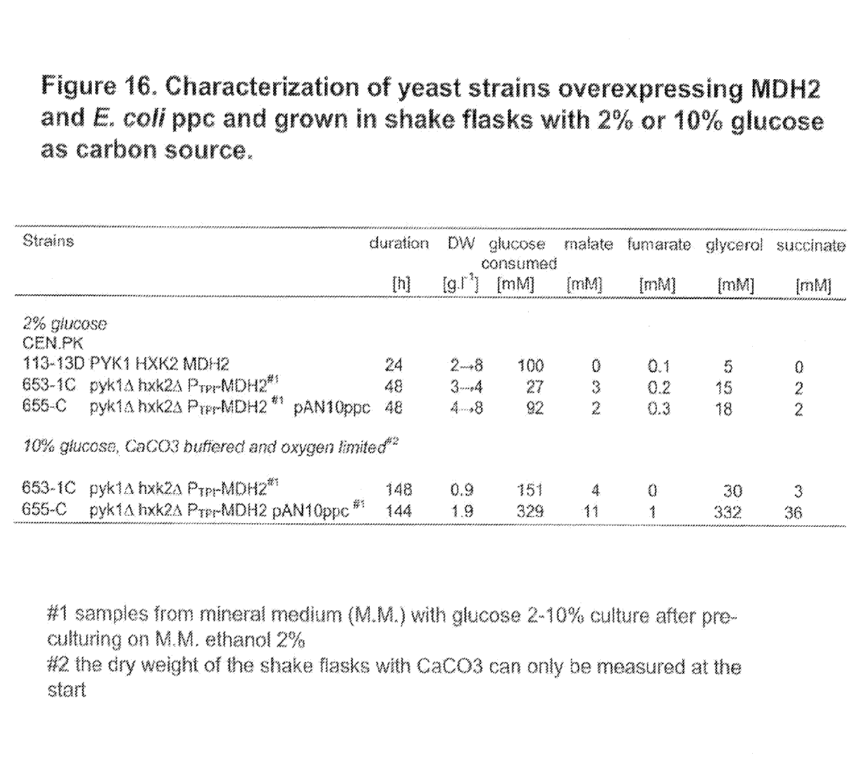

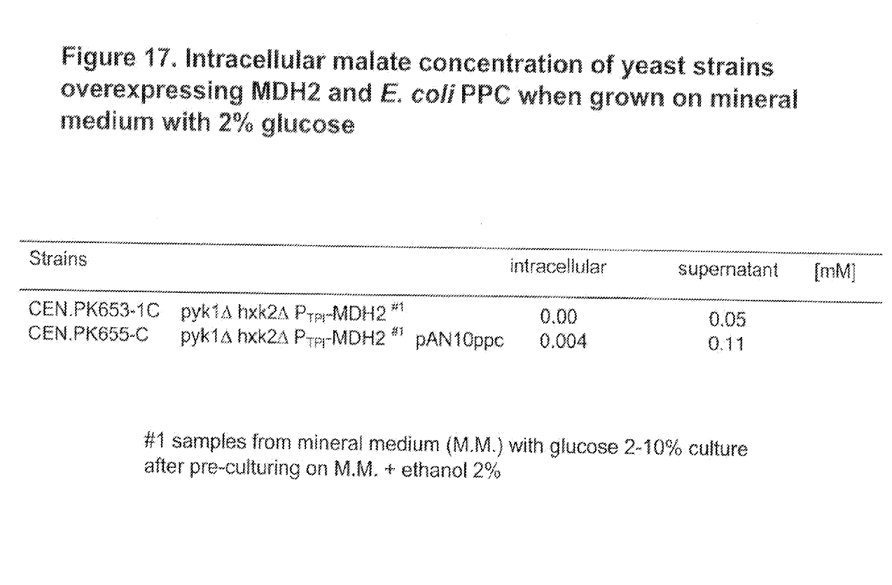

As can be seen in FIGS. 14 and 16, strains with the P_{TPI}-MDH2 construct did not show an increase in the total in vitro malate-dehydrogenase activity or an increased malate production when compared to wild-type strain, CEN.PK 113-13d. To see if the lack of malate production was due to a transport problem, the intracellular malate concentrations of shake flask grown strains on mineral medium with glucose was also measured. Samples were prepared as in examples 9A and 9B herein. No significant malate build-up was observed (see FIG. 17). The main products of the bioconversion were glycerol, carbon dioxide and succinate. The glycerol production in the pyk1Δhxk2ΔpAN10ppc P_{TPI}-MDH2 strain on 10% glucose in an oxygen limited environment obtained mol per mol ratios for glucose versus glycerol resulting in 0.332 M glycerol.

A lack of observed increased malate dehydrogenase activity may have occurred because Mdh2 is actively degraded when grown on glucose (Minard and McAlister-Henn (1992) J Biol Chem. August 25; 267(24):17458-64).

### Example 9A

### Preparation of Samples for Intracellular Metabolite Measurements

Biomass samples (4 ml of a 4 g dry weight/1 suspension) were taken from an anaerobic fermentation assay and immediately quenched with 20 ml 60% methanol at −40° C. After washing the cells twice with cold 60% methanol, intracellular metabolites were extracted by resuspending the cell pellets in 5 ml of boiling 75% ethanol and incubating them for 3 min at 80° C. Cell debris and intracellular metabolites were dried at room temperature with a vacuum evaporator (Savant Automatic Environmental SpeedVac® System type AES 1010). Finally, 0.5 ml of demineralized water was added. The resulting suspension was stored at −20° C. Before metabolite analysis, the suspension was centrifuged.

### Example 9B

### Quantification of Organic Acids, Glucose and Glycerol

Organic acids, glucose, and glycerol levels were quantified from broth samples using HPLC analysis. The instrumentation for detection was comprised of a Waters 717 Plus auto sampler fronting a Waters 515 pump, which was coupled to both a Waters 2414 refractive index (RI) detector and a Waters 2487 UV detector. An Aminex HPX-87H ion exclusion column (300 mm×7.8 mm, Bio-Rad), with Aminex HPX-87H guard column (20 mm×7.8 mm guard column, Bio-Rad), was used for separation. UV detection was typically employed to quantify organic acids, whereas RI detection enabled quantification of dextrose and glycerol; in many instances, malic acid detection also was performed using RI detection due to the overlap of malic acid and pyruvic acid peaks in chromatograms from UV detection.

Samples were prepared from HPLC analysis by first centrifuging (3600 rpm) harvested shake flask cultures and transferring supernatant to a fresh Eppendorf tube. Samples were diluted 50-fold into mobile phase, and the resulting preparations were loaded onto the 96 vial autosampler carousel, which is maintained at 15° C. 20 μL of diluted sample is used for instrument injection.

An isocratic separation was performed at 30° C. using 0.05% trifluoracetic acid as the mobile phase at a flow rate of 0.6 mL/min (1400 PSI as high pressure limit). UV detection was performed at 210 nm.

### Example 10

### Overexpression of Modified MDH Isoenzymes

MDH containing plasmids were constructed similar to those described in McAlister-Henn et al (1995) J Biol Chem. 1995 270:21220-5 and Small and McAlister-Henn (1997) Arch Biochem Biophys. 344:53-60. The first was a MDH1 gene from which the first 17 amino acids were removed, MDH1ΔL. Deletion of the first 17 amino acids was expected to allow partial cytosolic relocation with much of Mdh1ΔL still localizing to its normal compartment, the mitochondria. The second construct was the MDH3 gene from which the 3′ SKL sequence was removed, MDH3ΔSKL. Mdh3ΔSKL was expected to localize to the cytosol instead of the peroxisome. Both MDH constructs were expressed from the TDH3 promotor.

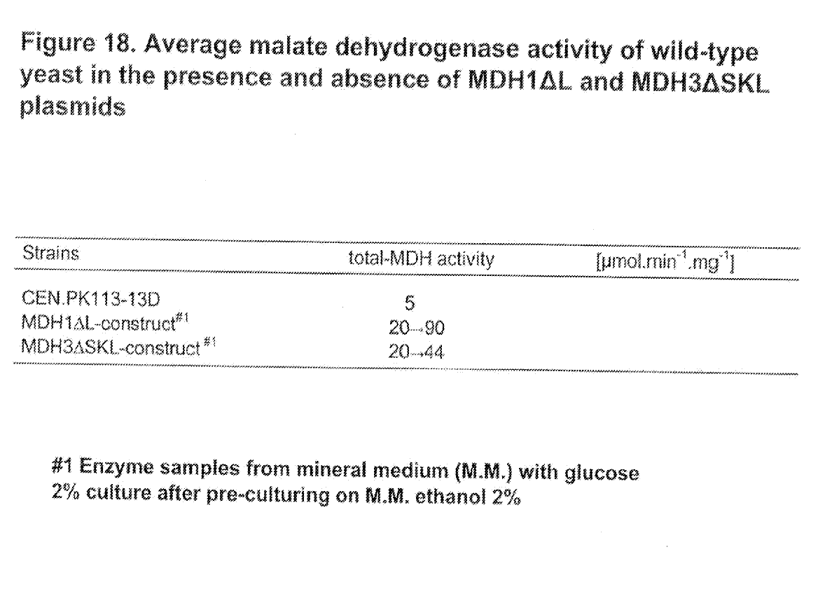

The total in vitro Mdh activity measured in strains with these constructs was over 4 to 20 fold that of a wild-type S. cerevisiae strain (CEN.PK113-13D). In shake flask fermentations on glucose, the enzyme activity varied between 20 to 90 μmol·min⁻¹·mg·protein⁻¹ (FIG. 18).

The sub-cellular fractionation of a wild-type strain, expressing Mdh3ΔSKL from the TDH3 promoter, grown in a nitrogen-limited continuous culture, showed that over 60% of the total Mdh activity was cytosolic. The rest of the activity was associated with the membrane fraction, which includes the mitochondria and peroxisomes.

Additional proof for the localization of the mutated Mdh enzymes was obtained by complementing mdh mutants. A S. cerevisiae strain with a deletion of the MDH1 gene, coding for mitochondrial Mdh, cannot grow with acetate as the carbon source, while deletion of the MDH2 gene, coding for the cytosolic Mdh, results in an inability to grow on mineral media with acetate or ethanol as the carbon source. Transformation of an mdh2 strain with plasmids containing MDH1ΔL and MDH3ΔSKL showed that both could complement the phenotype and therefore both are active in the cytosol. Transformation of an mdh1 mutant with the same constructs showed that MDH3ΔSKL could not complement the mdh1 phenotype (no growth on acetate) while MDH1ΔL could. Therefore Mdh3ΔSKL is only active in the cytosol, not in the mitochondria, while MdhΔL is active in both compartments.

Cultivation of S. cerevisiae CEN.PK113-32D (wt) with p425GPDMDH3ΔSKL (2μ plasmid, LEU2, TDH3 promotor (GPD is glyceraldehyde-3-phosphate dehydrogenase (TDH3)), overproduction MDH3 without terminal SKL sequence) was performed in continuous culture. The cultivations were performed at a growth rate of 0.1 h⁻¹ under nitrogen-limited conditions at pH 5. Metabolite measurements were performed as described in Examples 9A and 9B herein. The malate production did not exceed those found in wild-type S. cerevisiae strains without the MDH3ΔSKL construct, namely 0.03 g·l⁻¹. The carbon recovery was 97% yielding a stoichiometric balance of:

C₆H₁₂O₆ (glucose)+0.034 NH₃ (ammonia)+0.8O₂→0.71CH₁₈O_{0.5}N_{0.2}(biomass)+2.2CO₂+0.02C₃H₈O₃(glycerol)+0.01C₄H₆O4(succinate)+0.002C₄H₆O₅(malate)+0.09C₃H₄O₃(pyruvate)+1.4C₂H₆O+1.6H₂O.

Therefore, although in vitro studies showed MDH1ΔL and MDH3ΔSKL increase malate dehydrogenase activity, cultivation of the strains yielded carbon dioxide and biomass as main products and no significant malate production was observed. Further genetic and/or other manipulations (e.g. further comprising malic acid transporter polypeptides) in the context of MDH1ΔL and MDH3ΔSKL comprising strains may yield strains with increased observable malate production.

### Example 11

### Characterization of a Strain Comprising E. coli PEP Carboxylase and P_{TPI}-MDH2

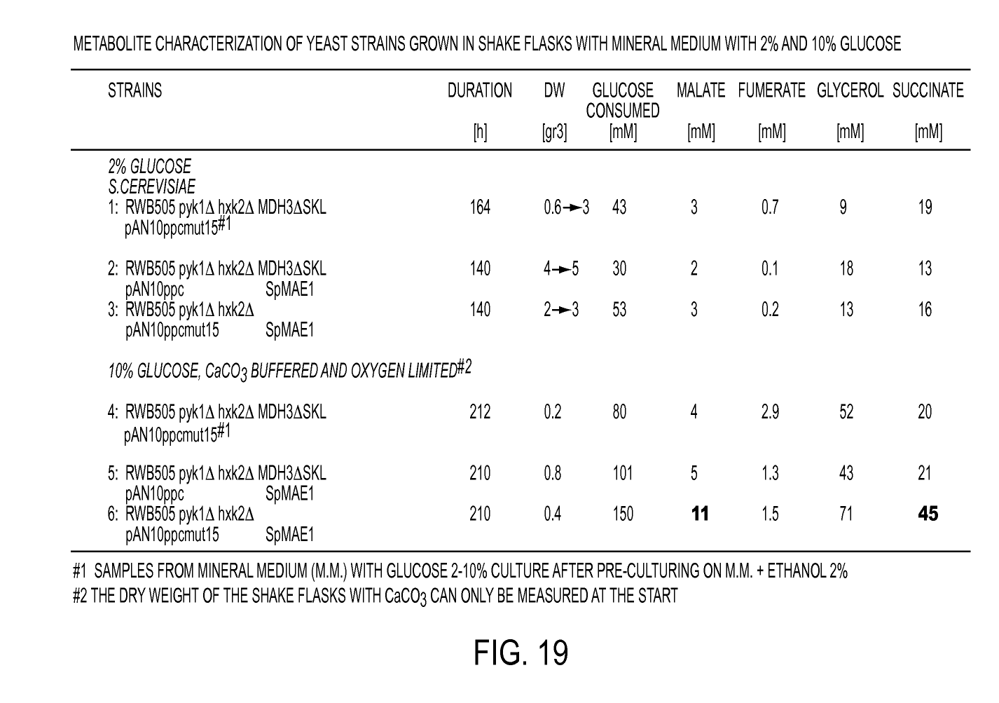

Quantitative characterization of CEN.PK655-1C (pyk1Δhxk2ΔP_{TPI}-MDH2 pAN10ppc) was performed in batch cultivations under oxygen limitation in both shake flask and bioreactors (See FIGS. 17 and 19). Inoculation in a batch bioreactor enabled CO₂ production measurement. The carbon balance showed that 98% of the consumed carbon could be traced, resulting in a stoichiometric balance for the oxygen limited phase of:

C₆H₁₂O₆(glucose)+0.09NH₃(ammonia)+XO₂→0.71CH₁₈O_{0.5}N_{0.2}(biomass)+2.16CO₂+0.02C₃H₈O₃(glycerol)+0.02C₄H₆O₄(succinate)+0.002C₄H₆O₅(malate)+0.02C₃H₄O₃(pyruvate)+5.3H₂O.

The oxygen transfer during the oxygen limited phase was estimated at 1.4 mmol·g⁻¹·h⁻¹. No holes in the carbon balance were observed and the main products were carbon-dioxide and biomass. Intracellular malate concentrations did not show an elevated malate concentration, suggesting malate transport was not problematic (see FIG. 17).

### Example 12

### Combining E. coli PEP Carboxylase with the MDH1ΔL and MDH3ΔSKL Alleles

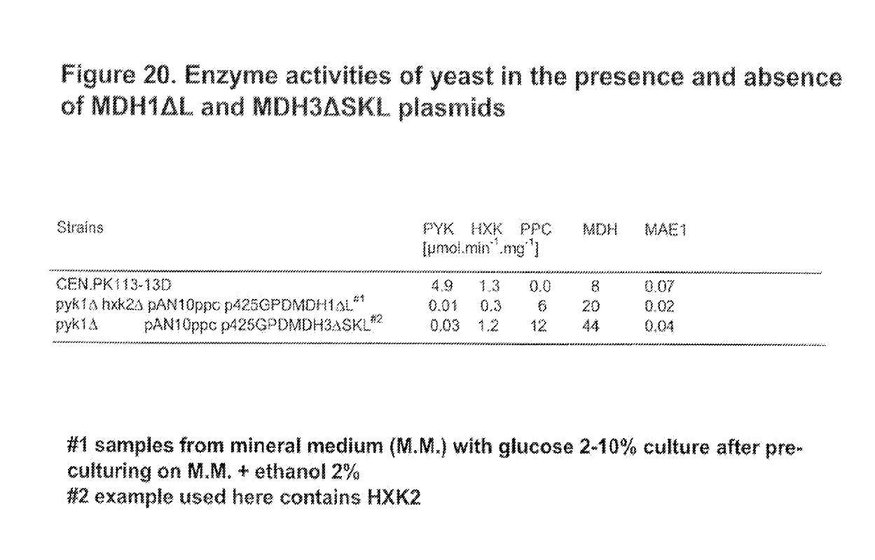

Strains comprising E. coli PEP carboxylase with Mdh isoenzyme alleles MDH1ΔL or MDH3ΔSKL were made and tested. Both were expressed from the TDH3 promoter and used to transform strains with disruptions in pyk1 and hxk2. The in vitro enzyme activities measured from extracts of MDH1ΔL and MDH3ΔSKL expressing strains that also expressed E. coli PEP carboxylase were 6 to 12 μmol·min⁻¹·mg·protein⁻¹ for E. coli PEP carboxylase and 20 to 40 μmol·min⁻¹·mg·protein⁻¹ for malate dehydrogenase, respectively (see FIG. 20).

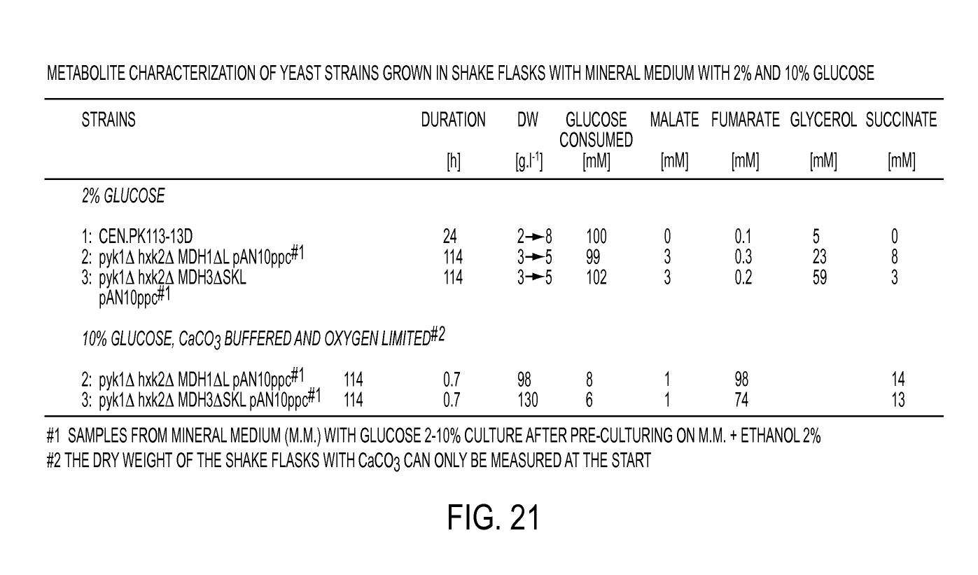

The strains were characterized in shake flask on 2% and 10% glucose containing mineral medium (see FIG. 21). A batch fermentation in a bioreactor with the RWB505 strain (pyk1Δ hxk2Δ pAN10ppc p425GPDMDH3ΔSKL) was conducted. Over the total fermentation 106% of the carbon could be accounted for with production of glycerol, succinate and biomass. For the oxygen limited phase this yielded a stoichiometric balance of:

0.22C₆H₁₂O₆(glucose)+0.034NH₃(ammonia)0.11O₂→0.17CH_{1.8}O_{0.5}N_{0.2}(biomass)+0.53CO₂+0.106C₃H₈O₃(glycerol)+0.006C₄H₆O₄(succinate)+0.001C₄H₆O₅(malate)+0.76H₂O.

Thus, despite the presence of high in vitro activities for ppc and Mdh, significant malate production was not readily observed. In efforts to further increase malate production, strains were generated that additionally contained deletions in either MAE1 (encoding malic enzyme), FUM1 (encoding fumarase), GPD1 and GPD2 (encoding glycerol-3-phosphate dehydrogenases), and PYK1 and PYK2 (encoding both pyruvate kinase enzymes in yeast). None of these mutations were shown to provide a benefit regarding malic acid production, at least in the genetic and growth contexts in which they were tested.

### Example 13

### Wild-Type and Mutant E. coli PEP Carboxylase Sensitivity to Malate

Wild-type and E. coli PEP carboxylase mutants were analyzed for inhibition in the presence of malate. Overproduction of E. coli PEP carboxylase was achieved using the pAN10ppc plasmid (Flores and Gancedo (1997) FEBS Lett. 412: 531-534), containing E. coli ppc gene behind the S. cerevisiae ADH1 promoter. Two amino acid changes, K620S and K773G, of E. coli PEP carboxylase have been reported to affect the inhibition of E. coli PEP carboxylase by aspartate and malate (Kai et al (2003) Arch Biochem Biophys. 414:170-9). Oligonucleotide-based site-directed mutagenesis was performed to generate ppc alleles that encoded putative malate-insensitive ppc polypeptides. Two oligonucleotides were designed in order to introduce both these mutations in plasmid pAN10ppc. Both mutant plasmids, pAN10ppcmut5 and pAN10ppcmut10 were introduced into wild-type S. cerevisiae strain, CEN.PK113-5D.

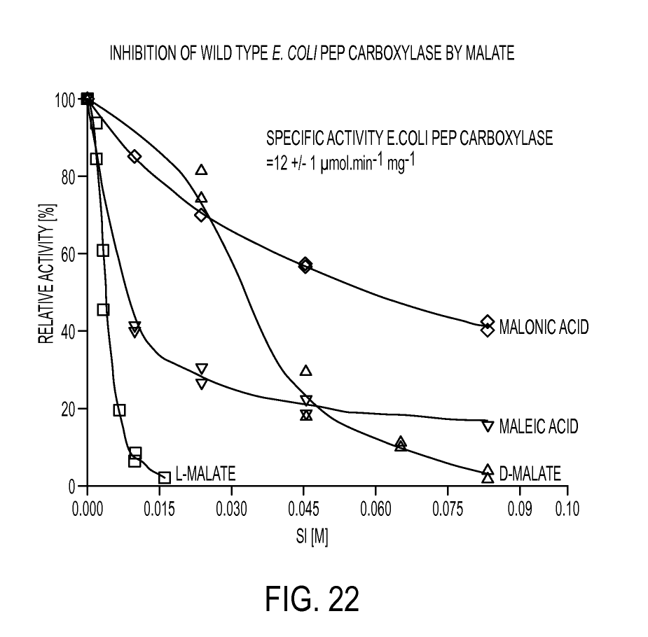

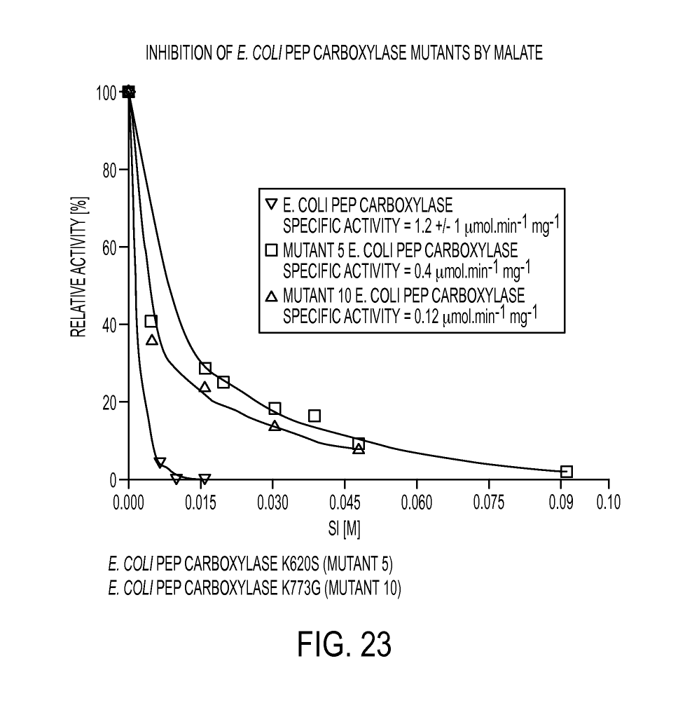

Cell extracts from glucose-grown shake-flask cultures were tested to determine the inhibition of malate as described in Examples 4 and 5 herein. The specific activity of wild-type E. coli PEP carboxylase is inhibited in the presence of malate (FIG. 22). In contrast, the specific activities of the pAN10ppcmut5 and pAN10ppcmut10 versus the wild-type E. coli PEP carboxylase were 0.4, 0.24 and 1.2 μmol·min⁻¹·mg·protein⁻¹ respectively. In the presence of 0.01 M malate, the wild-type E. coli PEP carboxylase was fully inhibited while both mutants, K620S (mutant 5) and K773G (mutant 10), still retained 40% of their initial activity (FIG. 23).

### Example 14

### Combination of E. coli Ppc Malate Insensitive Allele with Mdh3ΔSKL, pyk1Δ and Other Modifications

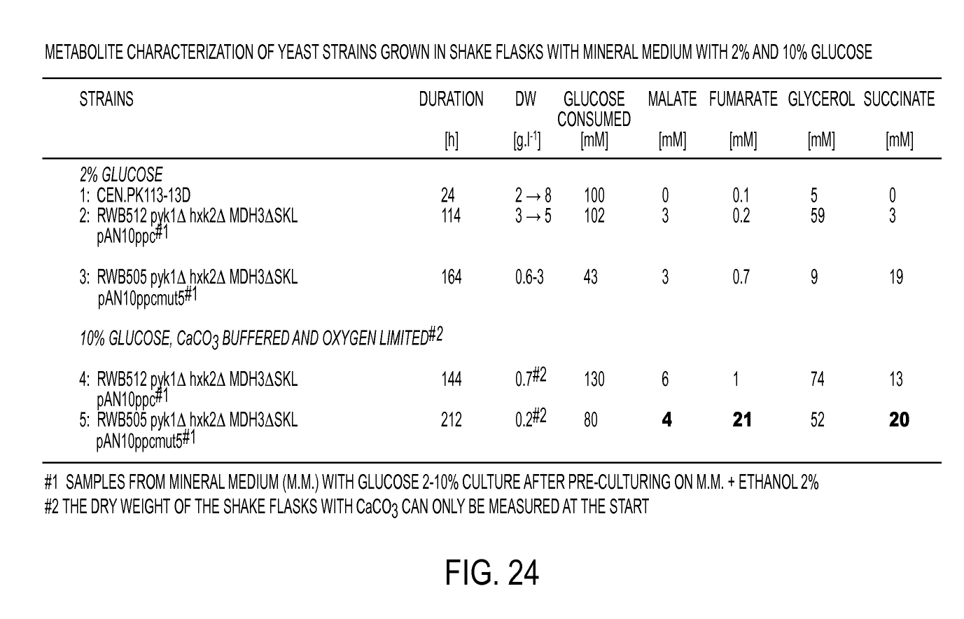

The S. cerevisiae RWB505 (pyk1Δhxk2Δ) was transformed with plasmids pAN10ppcMUT5 (encoding E. coli ppc K620S) and p425GPDMDH3ΔSKL. The strains were characterized in shake flask the on 2% and 10% glucose containing mineral medium (see FIG. 24). Comparison between a construct containing the K620S E. coli PEP carboxylase and a strain with wild-type E. coli PEP carboxylase showed no significant increase in the malate production. In contrast, succinate and fumarate levels increased and glycerol levels decreased. The differences may be partially explained by the lower dry weight at the start of the strain bearing the K620S E. coli PEP carboxylase. However, an effect on growth by intracellular build-up of metabolites in the K620S E. coli PEP carboxylase strain can not be excluded. Intracellular metabolite concentration analysis can be performed as in examples 9A and 9B herein to further test this. Additional genetic manipulation, for example expression of a malic acid transporter such as the S. pombe Mae1 gene can increase accumulation of extracellular malic acid, for example as in FIG. 19.

### Example 15

### Regulatory Sequences

Sequences which consist of, consist essentially of, and comprise the following regulatory sequences (e.g. promoters and terminator sequences, including functional fragments thereof) may be useful to control expression of endogenous and heterologous genes in engineered host cells, and particularly in engineered fungal cells described herein.

TDH3 promoter
(SEQ ID NO: 177)
cagtttatcattatcaatactcgccatttcaaagaatacgtaaataattaatagtagtgattttcctaactttatttagtcaaaaa
attagccttttaattctgctgtaacccgtacatgcccaaaatagggggcgggttacacagaatatataacatcgtaggtgtctgggtgaa
cagtttattcctggcatccactaaatataatggagcccgctttttaagctggcatccagaaaaaaaaagaatcccagcaccaaaatatt
gttttcttcaccaaccatcagttcataggtccattctcttagcgcaactacagagaacaggggcacaaacaggcaaaaaacgggcac
aacctcaatggagtgatgcaacctgcctggagtaaatgatgacacaaggcaattgacccacgcatgtatctatctcattttcttacacctt
ctattaccttctgctctctctgatttggaaaaagctgaaaaaaaaggttgaaaccagttccctgaaattattcccctacttgactaataagta
tataaagacggtaggtattgattgtaattctgtaaatctatttcttaaacttcttaaattctacttttatagttagtcttttttttagttttaaaacacca
gaacttagtttcgacggatt-3′
ADH1 promoter
(SEQ ID NO: 178)
cgccgggatcgaagaaatgatggtaaatgaaataggaaatcaaggagcatgaaggcaaaagacaaatataagggtcgaacga
a aaataaagtgaaaagtgttgatatgatgtatttggctttgcggcgccgaaaaaacgagtttacgcaattgcacaatcatgctgactctgt
ggcggacccgcgctcttgccggcccggcgataacgctgggcgtgaggctgtgcccggcggagttttttgcgcctgcattttccaaggttt
accctgcgctaaggggcgagattggagaagcaataagaatgccggttggggttgcgatgatgacgaccacgacaactggtgtcatt
atttaagttgccgaaagaacctgagtgcatttgcaacatgagtatactagaagaatgagccaagacttgcgagacgcgagtttgccgg
tggtgcgaacaatagagcgaccatgaccttgaaggtgagacgcgcataaccgctagagtactttgaagaggaaacagcaataggg
ttgctaccagtataaatagacaggtacatacaacactggaaatggttgtctgtttgagtacgctttcaattcatttgggtgtgcactttattatg
ttacaatatggaagggaactttacacttctcctatgcacatatattaattaaagtccaatgctagtagagaaggggggtaacacccctcc
gcgctcttttccgatttttttctaaaccgtggaatatttcggatatccttttgttgtttccgggtgtacaatatggacttcctcttttctggcaaccaa
acccatacatcgggattcctataataccttcgttggtctccctaacatgtaggtggcggaggggagatatacaatagaacagataccag
acaagacataatgggctaaacaagactacaccaattacactgcctcattgatggtggtacataacgaactaatactgtagccctagac
ttgatagccatcatcatatcgaagtttcactaccctttttccatttgccatctattgaagtaataataggcgcatgcaacttcttttctttttttttcttt
tctctctcccccgttgttgtctcaccatatccgcaatgacaaaaaaatgatggaagacactaaaggaaaaaattaacgacaaagaca
gcaccaacagatgtcgttgttccagagctgatgaggggtatctcgaagcacacgaaactttttccttccttcattcacgcacactactctct
aatgagcaacggtatacggccttccttccagttacttgaatttgaaataaaaaaaagtttgctgtcttgctatcaagtataaatagacctgc
aattattaatcttttgtttcctcgtcattgttctcgttccctttcttccttgtttctttttctgcacaatatttcaagctataccaagcatacaatcaactc
caagctggccgct-3′
TEF1 promoter
(SEQ ID NO: 179)
tagcttcaaaatgtttctactccttttttactcttccagattttctcggactccgcgcatcgccgtaccacttcaaaacacccaagc
acagcatactaaatttcccctctttcttcctctagggtgtcgttaattacccgtactaaaggtttggaaaagaaaaaagagaccgcctcgtt
tctttttcttcgtcgaaaaaggcaataaaaatttttatcacgtttctttttcttgaaaatttttttttttgatttttttctctttcgatgacctcccattg
atatttaagttaataaacggtcttcaatttctcaagtttcagtttcatttttcttgttctattacaactttttttacttcttgctcattagaaagaaagca
tagcaatctaatctaagtttt-3′

### Example 16

### Pdc Strain Construction and Malic Acid Production Analysis

| | |
|---|---|
| CEN.PK182 | MATa pdc1::loxP pdc5::loxP pdc6::loxP |
| CEN.PK2-1D | MATα ura3-52 trp1-289 leu2-3,112 his3Δ1 |
| RWB837 | MATa ura3-52 pdc1::loxP pdc5::loxP pdc6::loxP |
| TAM | MATa pdc1::loxP pdc5::loxP pdc6::loxP ura3-52 |
| | [URA3 plasmid] |
| | *evolved for Glu⁺ (able to grow in the presence of |
| | glucose) Et^{ind} (Ethanol independent-able to grow in the |
| | absence of ethanol) |

CENPK182 was crossed to CEN.PK2-1D and MATα pdc1 pdc5 ura3 trp1 (MY2219, MY2223, and MY2243 [also his3],) and MATα pdc1 pdc5 pdc6 ura3 trp1 (MY2222, MY2242 [his3], and MY2246 [his3]) progeny were identified.

RWB837 was transformed with an episomal 2 micron URA3 plasmid (YEpLpLDH) bearing the lactate dehydrogenase gene from Lactobacillus plantarum to create RWB876. RWB876 was subjected to 26 transfers through lactic acid fermentation medium (70 g/L glucose; 5 g/L ethanol) to create m850. Forty-five passages of m850 through the same medium lacking ethanol led to the isolation of Lp4f.

m850 and Lp4f were cured of their YEpLpDH plasmid, rendered trp1 ΔhisG using a hisG-URA3-hisG cassette (i.e. excision of the URA3 marker was accomplished on minimal drop-out plates containing 5-fluoroorotic acid by recombination between the hisG repeats, resulting in the clean deletion of the TRP1 gene), and serially transformed with pRS2MDH3ΔSKL and YEplac112SpMAE1 to produce MY2271 and MY2308, respectively. CEN.PK182 was likewise rendered trp1 ΔhisG, and along with MY2219, transformed with the same pair of plasmids to create MY2277 and MY2279, respectively.

MY2308 was crossed to MY2223 and MY2243 and prototrophic Glu⁺ progeny were identified, including MY2518 and MY2524.

TAM was cured of an episomal URA3 plasmid, rendered trp1 ΔhisG using a hisG-URA3-hisG cassette, and serially transformed with pRS2MDH3ΔSKL and YEplac112SpMAE1 to produce MY2264. MY2223, MY2243, MY2222, MY2242, and MY2246 were mated with MY2264 to create the diploid strains MY2300, MY2301, MY2299, MY2294, and MY2302, respectively.

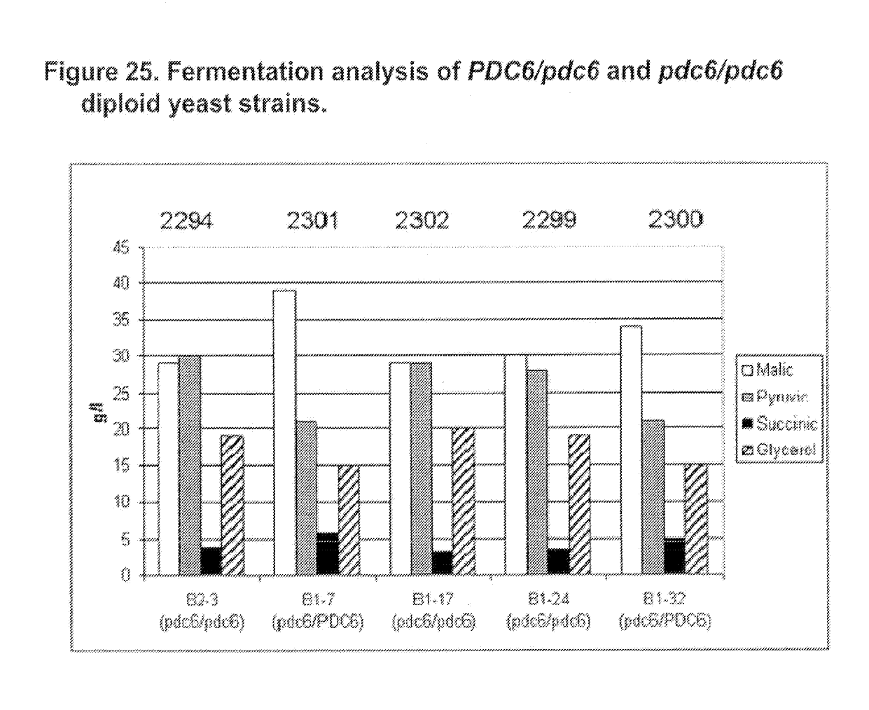

FIG. 25 shows fermentation results for these five diploids. It can be observed that the two PDC6/pdc6 strains produced higher malate to pyruvate ratios than that seen with the three pdc6/pdc6 strains. Ethanol levels were below detection.

MY2300 was sporulated and plated on minimal ammonia media supplemented with casamino acids (2 g/L), glycerol (10 g/L), and glucose (10 g/L), and prototrophic MATα segregants, including MY2433, were identified, and their pdc6 genotype determined by PCR analysis.

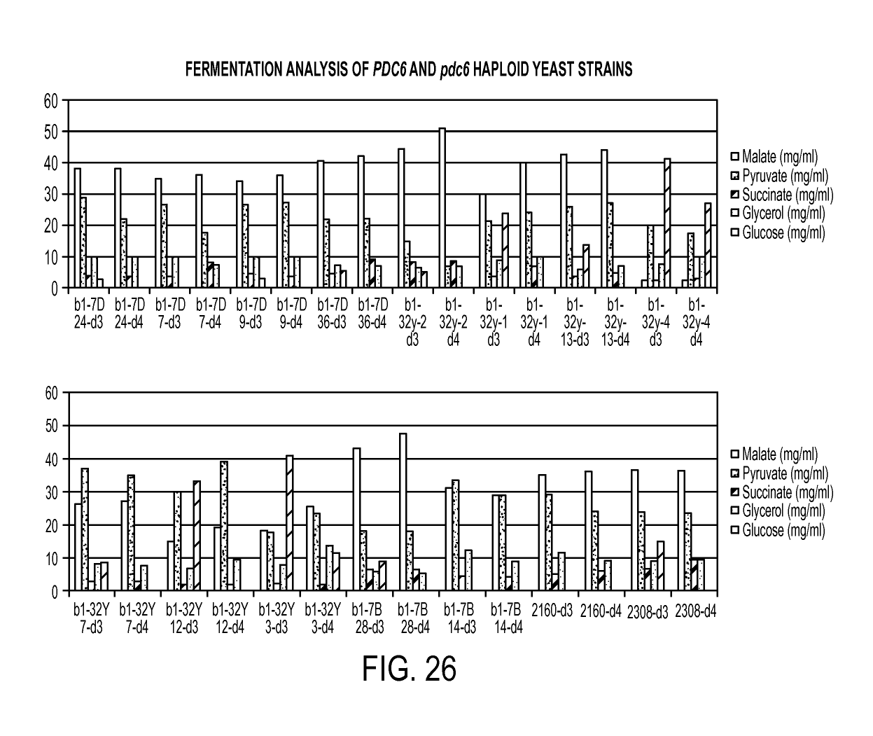

FIG. 26 shows fermentation results for 13 progeny from this cross. It can be seen that the on average pdc6 progeny produced a lower ratio of malate to pyruvate than did the PDC6⁺ progeny.

RWB961, MY2264, MY2271, MY2277, MY2279, MY2308, MY2433, MY2518, and MY2524 were compared in multiple fermentations. It was found that MY2433 and MY2518 were capable of producing in excess of 50 g/L malic acid (from 100 g/L glucose), and that MY2308 could produce up to 35 g/L. MY2271, MY2277, and MY2279 produced malic acid at a level not quite approaching that seen with the TAM derivatives RWB961 and MY2264 (20-30 g/L).

### Example 17

### Malate Dehydrogenase Variant

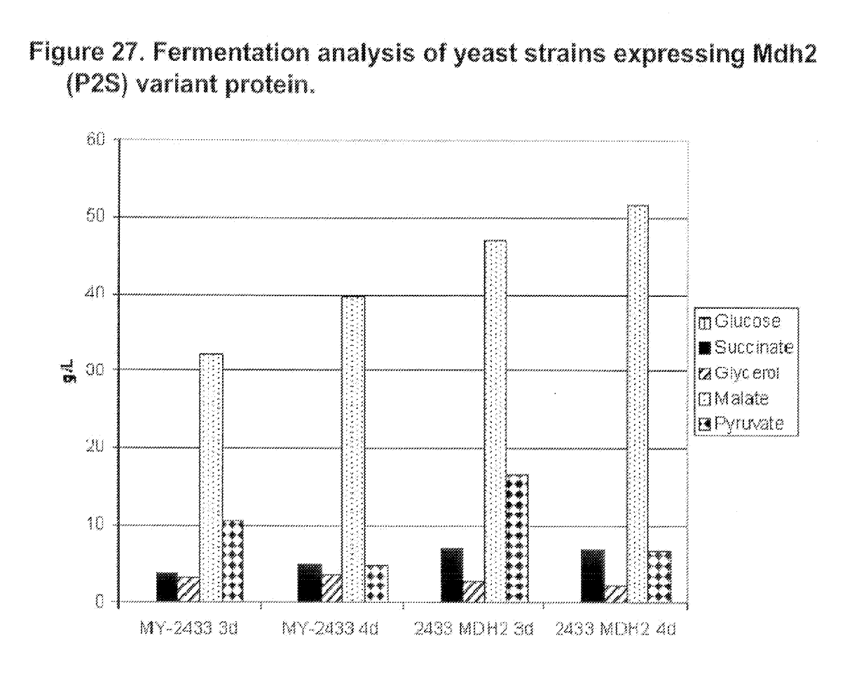

In order to create a variant of Mdh2 (MDH2-P2S) not subject to catabolite inactivation, we engineered a mutation in the coding sequence that encodes a serine rather than a proline at the second position after the start codon. M05448 (5′-CACACACTAGTAGTAACATGTCTCACTCAGTTACACCATCC (SEQ ID NO: 180)) and M05449 (5′-CACACCTCGAGTTAAGATGATGCAGATCTCGATGCA (SEQ ID NO: 181)) were used to amplify a 1.0 kb fragment from S. cerevisiae genomic DNA by PCR, which was subsequently cleaved with Xhol and Spel, and ligated to M/ui-Xbal-cleaved pRS2MDH3L1SKL along with the 178 bp M/ui-Xhol CYC1t fragment from pRS413TEF, to create pMB4978. When strains carrying pMB4978 were compared with isogenic strains carrying pRS2MDH3L1SKL in shake flask fermentations, a consistent improvement was seen. For example, when MY2433 was cured of pRS2MDH3L1SKL and transformed with pMB4978, the resulting strain produced >25% more malic acid in a four-day fermentation (FIG. 27); other experiments and strain backgrounds gave similar results.

### Example 18

### Sequence of PYC2-ext

In order to create a variant of pRS2MDH3L1SKL in which the encoded Pyc2 protein (PYC2-ext) possesses the five amino acid carboxy terminal extension that is found in other common wild type yeast strain backgrounds, we engineered a frameshift mutation in PYC2 by inserting an additional cytosine residue into a consecutive series of four cytosine residues found near the 3′ end of the coding strand ( . . . ATCCCCAAAAA . . . (SEQ ID NO: 182)). M05265 (5′-CACACCGTCTCAGGGGATGGGGGTAGGGTTTC-3′ (SEQ ID NO: 183)) and M05183 (5′-GCCAAGGATAATGGTGTTGA-3′ (SEQ ID NO: 184)) were used to amplify a 1.3 kb fragment from pRS2MDH3L1SKL DNA that was subsequently cleaved with Eag1 and BsmBI. M05266 (5′-CACCGTCTCACCCCAAAAAAAAAGTAATTTTTACTCGTT-3′ (SEQ ID NO: 185)) and M05186 (5′-GCAGCAATTAGTTGGCGACA-3′ (SEQ ID NO: 186)) were used to amplify a 300 bp fragment from pRS2MDH3L1SKL that was subsequently cleaved with BsmBI and M1u1. These fragments were ligated to the large fragment of Eag1- and M/u1-cleaved pRS2MDH3L1SKL to create pMB4968. The PYC2-ext allele in pMB4968 encodes a protein with the carboxy terminal sequence . . . EETLPPSPKKVIFTR(stop) (SEQ ID NO: 187), instead of the sequence . . . EETLPPSQKK(stop) (SEQ ID NO: 188) encoded by the PYC2 gene of pRS2MDH3L1SKL. When strains carrying pMB4968 were compared with isogenic strains carrying pRS2MDH3L1SKL in shake flask fermentations, slightly higher amounts of malic acid were detected with pMB4968 (PYC2-ext). Other factors such as increasing biotinylation capacity or supplemental C02 could increase the utility of this allele.

### Example 19

### Phosphoenolpyruvate Carboxykinase (PEPck) DNA and Strain Construction

A gene encoding the phosphoenolpyruvate carboxykinase (PEPck) protein corresponding to that encoded by Actinobacillus succinogenes was constructed by de novo gene synthesis as follows. The sequence

(SEQ ID NO: 189)
ttctagaaacaaaatgactgatttgaataaactggttaaggaattaaacgatttgggactgacagatgtaaaagaaatcgta
tataatccaagttacgagcaattattcgaagaagaaactaagccaggattagaaggatttgacaagggtacattgacgacactaggg
gccgtagcggtagatacaggaatttttactggcagatctcccaaagataaatatatagtgtgcgatgaaactacgaaagataccgtatg
gtggaatagcgaagcagcaaaaaatgataacaaaccaatgactcaagaaacatggaaaagcttaagagaattagttgctaaaca
attatccggtaaaagactatttgttgttgaaggttattgtggtgcgagcgagaaacatagaatcggtgtgagaatggtgacggaggtag
cttggcaagctcattttgttaaaaatatgtttataagaccaacagatgaagaattgaaaaactttaaagccgactttacagtcctaaacgg
tgctaaatgtactaatccaaattggaaggagcaaggtttaaattctgaaaatttcgtagcgtttaatattacagaaggaattcaattaatag
gaggtacatggtacggaggtgaaatgaaaaagggtatgttttcgatgatgaattatttcctaccgttaaaaggtgtagcatctatgcattg
cagtgctaacgttggaaaggacggtgatgttgctattttttttggtttatccggcacaggaaaaacaaccctatcaactgacccaaaaag
acagctaattggtgatgatgaacatggatgggacgaatcaggtgtctttaatttcgaaggtgggtgttatgcaaagacaattaatctatcc
caagaaaatgaaccagatatttacggtgctattagaagagatgctttgttagagaacgttgtagtaagagctgacggttccgttgattttg
atgatggctccaaaaccgaaaatactagagtatcttatccaatctaccacatagataacattgttagaccagtatctaaggccggacat
gctactaaggtcatattcctaaccgcagatgctttcggtgttcttcccccagtaagtaagttaactcccgaacaaaccgaatactatttcct
aagtggatttacagctaaattagccggcacagaaagaggcgtcacagaaccaactccaaccttctccgcttgttttggggccgcattttt
gtcattgcatccaattcaatatgcagatgtattagtagaacgtatgaaagctagcggcgcagaggcttatttggttaacacaggatgga
atggaacaggtaaaagaatttctattaaagacacaagaggaataattgacgctatattagatggaagtattgaaaaggctgaaatgg
gcgaactaccaatattcaacttagctatacctaaagctttaccaggggtagatccagcaattttagacccaagagacacgtatgcagat
aaagctcaatggcaagtaaaggcagaagacttagctaacagattcgtaaaaaatttcgtaaagtacaccgctaacccagaagctgc
taaattggttggagctggacccaaggcctaactcgag-3′

was synthesized, cleaved with XbaI and XhoI, and ligated to pRS416TDH3, pRS416ADH1, and pRS416TEF1 to produce pMB4917, pMB4920, and pMB4919, respectively.

Primers M05198 (5′-CACACTCT AGAAACAACATGCAGA TCAACGGTA TTACCCCG-3′_ (SEQ ID NO: 190)) and M05199 (5′-CACACCTCGAGTTACCGCTTCGGTCCTGCTTTCAC-3_ (SEQ ID NO: 191)) were used to amplify Erwinia carotovora DNA (ATCC 33260), and the resultant 1.6 kb fragment was cleaved with Xbal and Xhol and ligated to pRS416TDH3, pRS416ADH1, and pRS416TEF1 to produce pMB4922, pMB4926, and pMB4925, respectively.

Primers M05196 (5′-CACACTCT AGAAACAACATGTTAAGTCGTATTGAACAAGAAC-3′_{—} (SEQ ID NO: 192)) and M05197 (5′-CACACCTCGAGTTAAAGTTTCGGACCTGCCGCAACT-3′_{—} (SEQ ID NO: 193)) were used to amplify Actinobacillus pleuropneumoniae DNA (ATCC 27088), and the resultant 1.6 kb fragment was cleaved with Xbal and Xhol and ligated to pRS416TDH3, pRS416ADH1, and pRS416TEF1 to produce pMB4914, pMB4916, and pMB4915, respectively.

These nine pck plasmids were found to suppress the gluconeogenic defect of a S. cerevisiae pck1 mutant devoid of PEPck activity. Moreover, a pck1 strain harboring pMB4915 was found to contain PEPck activity when grown on glucose as well as on ethanol, suggesting that the catabolite inactivation of native S. cerevisiae PEPck is not observed with A. pleuropneumoniae PEPck.

None of the plasmids complemented the anaplerotic defect of S. cerevisiae pyc1 pyc2 double mutants. Neither the insertion of the SacI-XhoI pck expression cassette from pMB4915 (in both orientations) or from pMB4919 (in one orientation) into pRS2MDH3ΔSKL by blunt end ligation into the unique M/uI site, nor the replacement of the resident pRS2MDH3ΔSKL PYC2 gene by blunt end ligation of these cassettes into PstI- and BsiWI-cleaved pRS2MDH3ΔSKL, yielded significant malate yield improvement over that seen with pRS2MDH3ΔSKL in shake flask fermentations. Further experimentation such as one or more of manipulation of strain backgrounds, introduction of novel pck alleles, CO₂ supplementation, and anaerobic conditions can be tested to improve the anaplerotic capacity of these enzymes.

### Example 20

### Organic Acid Transporters

Genes encoding putative aluminum-activate organic acid transporters (Oat_{Mal}) proteins corresponding to those encoded by Brassica napus and Triticum secale were constructed by de novo gene synthesis as follows. Two sequences,

(SEQ ID NO: 194)
ttctagaaacaaaatggaaaaattgcgtgaaatagttagagagggaagaagagttggcgaagaggatcccagaagaat
tgtacactcatttaaagttggagtcgcgttggttttagttagctcattttactactatcaaccatttggtccatttactgactactttggtataaatg
cgatgtgggccgtaatgaccgtcgttgttgtttttgaattttctgtcggagctactttaagtaaaggattaaatagaggtgtcgcaactttagt
cgcaggaggcctagcgttaggagcacatcaattggcttcattatcaggaaggactatagaacccattctattggctacttttgtatttgtta
cagcagcacttgctacctttgttcgttttttcccgagagttaaggctacatttgattatggaatgctaattttcattctaacttttagcttaatttcctt
atcccagtttagagacgaagaaatattagacttagctgaatcgagattatcaactgtattagttggcggggttagttgtattttaatttccata
tttgtttgtccagtttgggccggtcaggacttacattcactattagtttcaaaccttgatactctaagccactttttacaagaattcggtgatga
atatttcgaagcgagaacatatggtaatattaaagttgttgaaaagagaagaagaaaccttgagagatacaaatcagtgctaaactca
aaatccgatgaagattccctagcaaatttcgcaaaatgggaaccaccacatggcaaattcggttttagacatccatggaaacaatattt
agtcgtcgcagctttagttagacagtgcgctcatagaatagatgctttaaactcttatattaattcaaattttcaaatcccaatcgatataaa
aaagaaattggaagaaccattcaggagaatgtcattagaatctggaaaagcaatgaaagaagcttcaattagtctgaaaaaaatga
ccaaatccagcagttacgatatccatataattaatagccaatctgcatgcaaagccttatctaccttgttaaaatctggtatattaaacgac
gttgagccattacaaatggtgagtttactaactacagtttctttattaaatgacatagttaacataacagaaaaaataagtgaatctgtgag
agaattggcttccgctgctagattcaggaataaaatgaaacctactgaaccaagtgtttccctaaaaaagttagattcaggttctacagg
atgtgcaatgccaataaattcaagggatggtgatcatgttgtaaccatattacttagtgacgatgataaagatgatatagatgatgacgat
acttcaaatatagtactagacgatgacactattaatgaaaagtctgaagatggtgaaatacatgtacaaaccagttgtgtaagagaggt
gggaatgatgcctgaacattcacttggtgtaagaatattgcaaatttaactcgag-3′ (B. napus (B.n.))
(SEQ ID NO: 195)
ttctagaaacaaaatggatattgatcatggaagagaaatagatggagaaatggtttctactattgcgtcatgcggcttgttattg
cattccttattagcaggtttcgcaagaaaggtcggtggtgctgccagagaagatcccagaagagttgctcattcattaaaagttggtcta
gcattggctctagtttcagctgtttactttgtaacaccattattcaacgggttaggcgttagtgcaatttgggctgttcttaccgtagtcgtcgtt
atggagtttaccgtcggtgcaactttaagtaaaggtttaaatagagctttggcaactttagtcgcaggatgtattgctgtcggagcccatca
attagcagaattaacagaacgttgttcagatcaaggggaaccagttatgttgacagtattagttttttttgtcgcatcagcagcaacatttctt
agattcattcccgaaatcaaagcaaaatatgactatggcgtaactatttttatactaactttcggtttagttgctgtttcgtcttacagagtgga
agaacttattcaattagctcatcaaagattttacacaattgtcgtcggagtatttatatgtctatgcacaacggtatttttatttcctgtttgggcc
ggagaggacgtccataaattagcttcatcaaatttagggaaattagcgcaatttattgaaggtatggaaacaaactgttttggcgaaaa
caacatagctatcaatttagaaggaaaagattttttacaagtatacaaatcggttctgaattcaaaggccactgaagattctttatgcacttt
tgcaagatgggaaccaagacatggtcagtttagatttagacacccctggtctcaatatcaaaaattaggtacactgtgtagacaatgcg
catcatcaatggaagctttagctagttacgttattaccaccacaaagactcaataccccgcagctgcaaatccggaactttcttttaaagt
cagaaaaacatgtcacgaaatgtctactcatagtgctaaagttttaagaggtttagaaatggcaatacgtacaatgacagtcccatactt
agccaacaatacagtcgtagttgcaatgaaggccgccgagagattaagatcagaattagaagataacgctgcacttttacaggtaat
gcatatggctgttactgctacgttacttgccgatttagtcgatagagtcaaagaaatcacagaatgtgttgatgttttagcaagattagccc
attttaaaaatcctgaagatgcaaaatacgcaatcgttggtgctttaactagaggaatagatgatcctttgcctgatgtagttatattataac
tcgag-3′ (T. secale (T.s))

were synthesized, cleaved with XbaI and XhoI, and ligated to pRS416TDH3, pRS416TEF1, and pRS416ADH1 to produce pMB4943 (TDH3-B.n.), pMB4944 (TEF1-B.n.), pMB4945 (ADH1-B.n.), pMB4946 (TDH3-T.s.), pMB4947 (TEF1-T.s.), and pMB4948 (ADH1-T.s.); all are URA3-marked plasmids. In addition, analogous constructs were made in a TRP1-marked series of plasmids: pMB4950 (TDH3-B.n.), pMB4952 (TEF1-B.n.), pMB4954 (ADH1-B.n.), pMB4949 (TDH3-T.s.), pMB4951 (TEF1-T.s.), and pMB4953 (ADH1-T.s.).

Although no evidence for malic transport was observed when compared with the isogenic controls MY2308 (Lp4f [pRS2MDH3ΔSKL][YEplac112SpMAE1]) and MY2306 (Lp4f [pRS2MDH3ΔSKL][pRS424]) when tested in shake flask fermentations in the Lp4f background, further analysis including addition of aluminum cations, alleviation of possible cellular mislocalization, altered growth conditions or strain backgrounds can be tested.

### Example 21

### Overexpression of Biotin Protein Ligase

Addition of excess biotin (550 ng/ml vs. 50 ng/ml) in the fermentation medium consistently improved malic production in shake flask assays. In order to exploit the presumably high intracellular biotin pools under these conditions, enzymes responsible for biotinylating Pyc were overexpressed.

Primers M05442 (5′-CACACTCT AGAAACAAAA TGAATGTATTAGTCTA TAATGGCCC-3′ (SEQ ID NO: 196)) and M05443 (5′-CACACCTCGAGGGTAGACTCTTAACTCTGAACC-3′_{—}

(SEQ ID NO: 197)) were used to PCR amplify a 2.1 kb fragment (comprising the BPL 1 gene which encodes biotin protein ligase) from S. cerevisiae genomic DNA. The 2.1 kb fragment was subsequently cleaved with Xhol and Xbal and ligated to Xhoi-Xbal-cleaved pRS413TEF to create pMB4976. When introduced into MY2520, MY2522, MY2527, MY2528, and MY2486, no improvement of malate production in shake flask fermentation was observed under the conditions tested. Further experimentation including one or more of altering the Pyc substrate, increasing the intracellular biotin concentration, mutating the biotinylation site of the Arc1 protein (for example as described in example 26 herein), or deregulating the Bp11 enzyme by specific alleles can be tested.

Primers M05442 (5′-CACACTCTAGAAACAAAATGAATGTATTAGTCTATAATGGCCC-3′) and M05443 (5′-CACACCTCGAGGGTAGACTCTTAACTCTGAACC-3′) were used to PCR amplify a 2.1 kb fragment (comprising the BPL1 gene which encodes biotin protein ligase) from S. cerevisiae genomic DNA. The 2.1 kb fragment was subsequently cleaved with XhoI and XbaI and ligated to XhoI-XbaI-cleaved pRS413TEF to create pMB4976. When introduced into MY2520, MY2522, MY2527, MY2528, and MY2486, no improvement of malate production in shake flask fermentation was observed under the conditions tested. Further experimentation including one or more of altering the Pyc substrate, increasing the intracellular biotin concentration, mutating the biotinylation site of the Arc1 protein (for example as described in example 26 herein), or deregulating the Bpl1 enzyme by specific alleles can be tested.

### Example 22

### Overexpression of S. cerevisiae Carbonic Anhydrase

In order to increase the flux of C02-HC03-, the S. cerevisiae carbonic anhydrase (product of the NCE103 gene) was overexpressed. Primers M05257 (5′-CACACTCT AGAA TCAGAATGAGCGCTACCGAA TCTTC-3′ (SEQ ID NO: 198)) and M05258 (5′-CACACCTCGAGCTATTTTGGGGTAACTTTTG-3′ (SEQ ID NO: 199)) were used to PCR amplify a 700 bp fragment from S. cerevisiae genomic DNA. The fragment was subsequently cleaved with Xhol and Xbal, and ligated to Xhoi-Xbal-cleaved pRS413TEF to create pMB4958. When introduced into MY2520, MY2522, MY2526, and MY2486, no improvement of malate production in shake flask fermentation was observed under the conditions tested. Further experimentation including culturing NCE 103 overexpressing strains under conditions of lower pH or higher intracellular C02 can be tested.

### Example 23

### MTH1 Variant Construction and Analysis

Both TAM and m850/Lp4f strains were obtained as strains evolved to overcome phenotypes associated with substantial elimination of PDC polypeptide activity, failure to grow on glucose minimal medium without a C2 carbon source and intolerance of high level glucose. Evolution was by extensive continuous culturing, either by chemostat or by extensive serial passages. Genetic analysis of these strains revealed that a) the TAM Glu+ phenotype is dominant; b) the TAM Glu+ phenotype segregates as a single gene in a cross; and c) the Lp4f Glu+ phenotype segregates as two or more genes; and d) one of the Lp4f Glu+-conferring alleles is tightly linked to the TAM Glu+-conferring allele. Since dominant mutations in the MTH1 gene have been shown to suppress the Glu-phenotypes seen in several mutants, DNA was amplified from several strains with the primers M05522 (5′-CAATAGCGAAACCACAAGCAGC-3′ (SEQ ID NO: 200)) and M05523 (5′-GTCTCATCGCTAGAATATAGTGG-3′ (SEQ ID NO: 201)) to amplify a 848 bp 5′ fragment (from −72 to nt749) of MTH1, and the primers M05524 (5′-CTGTAACGGGCGTCCCAAG (SEQ ID NO: 202)) and M05525 (5′-CCTTGGGAATTTGGAGCTCC (SEQ ID NO: 203)), to amplify a 821 bp 3′ fragment (from nt561 to +107) of MTH1. Sequence analysis of the fragment generated with M05524 and M05525 from TAM, Lp4f, m850, and wild type CEN.PK revealed no polymorphisms or deviations from the published MTH1 sequence. However, the PCR fragment generated with M05522 and M05523 from TAM revealed a 225 bp deletion of an internal segment of the coding region, from nt169 to nt393, resulting in the in-frame deletion of aa57 to aa131. Sequence analysis of the 5′ fragment generated from Lp4f revealed a mutation at nt218 (AATGCTCCT----*AATGATCCT) corresponding to a lesion at aa73 (VNAPP----*VNDPP (SEQ ID NOS 204 and 205,respectively)). Restriction analysis of the 5′ fragment generated from the parent of Lp4f, m850, confirmed that it also harbors the A73D allele (loss of a Bsm1 site; gain of a Sau3A site). A CEN.PK wild type strain was shown to carry the published MTH1 sequence throughout.

Transformation of the Glu⁻ pdc1 pdc5 strains MY2218 and MY2219 with the 5′ fragment from TAM, carrying the 225 bp deletion (denoted MTH1ΔT), resulted in the appearance of colonies on YPD that could be shown by PCR to harbor MTH1ΔT, confirming that the deletion alone is sufficient to confer the ability to grow on glucose to Pdc-defective strains.

### Example 24

### Construction and Expression of Pyc and Bpl Polypeptides

Genes encoding Pyc and Bpl from Aspergillus niger and Yarrowia lipolytica, and a gene encoding Pyc from Nocardioides sp. are synthesized as follows:

(SEQ ID NO: 206)
actagtaaatatgtctaatgttccagaaactaaagtagatccttcattgtccacaccagaggtccctagtcaaggtttacatag
cagattggacaagatgagagctgattcatccatattgggaagtatgaacaaaatattagtggcaaatagaggtgaaatcccaattaga
atctttagaaccgcccacgagttatctatgcagactgttgctatctatgcacatgaggacagattgtcaatgcacagattcaaggccgat
gaggcttacgtaattggagacagaggaaaatatacacctgtccaagcatacttacaggtggacgagataatcgaaattgccaaggctcatg
gtgttaacatggtacacccaggatatggtttcttgtccgaaaatagtgagttcgcaagaaaagtcgaagaagctggaatggcctg
gattggtcctccacataacgttatagacagtgtcggtgacaaggtttcagcaagaaacttagctatcaagaacaatgtacctgtcgtgcc
aggaaccgatggtcctgttgaggacccaaaggatgccttgaaatttgtagaaaagtacggttatcctgtcattataaaagcagctttcgg
aggtggaggtagaggtatgagagttgtgagagagggagatgacatcgttgatgcctttaacagagcatccagtgaagctaagactgc
cttcggtaatggtacatgtttcattgaaagattcttagacaaaccaaaacatatagaggtacaattgttagcagatggacaaggtaatgt
cgtgcacttgtttgaaagagattgctctgttcagaggagacatcaaaaggtagtcgaaatcgctccagccaaagacttacctgtcgag
gtgagagatgcaattttggacgatgctgttagattagctgaagatgccaagtacagaaacgcaggaaccgctgagttcttggtagacg
agcaaaatagacactacttcattgagataaacccaagaatccaggtcgaacatactattacagaggaaataaccggtatcgatattgt
tgccgcacaaatacagattgctgccggtgcaactttagagcaattgggattaacacaagacaaaatctcaactagaggttttgctattc
agtgtagaataaccacagaagatcctgcaaagcaattccaaccagatactggaaaaatcgaagtctacagatctgctggaggtaat
ggagtaagattggacggtggtaacggatttgccggtgcaattatatcccctcactatgatagtatgttagtcaagtgctcatgttctggcac
cacattcgagatagccagaagaaagatgattagagccttggttgagtttagaataagaggagtcaagactaatattccattcttattggc
attattgacacatcctacctttatcgaaggaaaatgctggactacattcattgacgatactccatccttatttgacttgatgaccagtcagaa
cagggctcaaaagttattggcctacttagcagatttatgtgttaatggaacaagtataaaaggtcaggtaggtaaccctaagttaaagtc
tgaggtcgttatcccagtgttgaagaactccgaaggaaagattgtagattgtagtaaacctgacccagtcggttggagaaatatattagt
tgaacaaggtcctgaggctttcgccaaggcagtgagaaagaacgatggagttttggtaatggacactacctggagagatgctcatca
atcattattggctacaagagtcagaactaccgacttattggcaattgcaaatgaaacatctcacgctatgtccggtgcctttgcattagagt
gctggggaggtgctacttttgacgttgcaatgagattcttgtatgaagatccatgggacagattaagaaagatgagaaaagcagtgcc
aaatatcccttttcagatgttgttaagaggtgctaatggagtagcctactcatctttgccagataacgcaatagatcatttcgtcaagcaag
ctaaagacaatggtgttgatatctttagagtgttcgacgccttaaacgatttggatcaattaaaggtaggtgttgacgcagtcaagaaag
ctggaggtgttgtggaagcaaccgtatgttatagtggagatatgttgaatcctaagaagaagtacaacttagagtattacttggactttgtc
gatagagttgtagaaatgggcacccacatcttaggtattaaagatatggcaggaactttgaagccagctgccgcaaccaaattaatag
gtgctatcagagaaaagtatcctaatttgccaattcatgttcatacacacgactccgccggtactggagtggcatcaatggctgccgca
gctgaggccggtgcagatgtcgttgacgtggcttctaatagtatgtctggaatgacctcccagccttcaataagtgccttaatggcaacat
tggaaggaaaattatctactggtttggacccagctttagtaagagaattggatgcctattgggcacaaatgagattattgtactcatgcttc
gaggctgacttaaagggacctgatccagaagtctttcaacatgaaattcctggtggtcagttgacaaacttattgttccaagcccagcaa
gttggattaggtgagcaatggaaagaaactaagcaggcatatatcgctgccaatcaattgttaggagacattgtaaaagttaccccaa
catctaaggtggtcggtgatttggcacagtttatggtttccaacaaattaagttacgacgatgtgataaaacaggctggttcattggattttc
ctggatctgtattagacttctttgagggtttgatgggtcaaccatatggaggtttcccagaacctttaagaactgaagcattaagaggaca
gagaaagaaattaaccgagaggcctggaaaatccttgcctccagtcgattttgcagctgttagaaaagacttagaagaaagattcggt
cacatcacagagtgtgatattgccagttactgcatgtatcctaaggtatttgaagattacagaaagatagttgacaagtatggagatttgt
caattgtgccaactagattattcttggaagcacctaaaaccgacgaggaattttctgtcgaaatcgagcaaggtaagacattaatattgg
ctttaagagctattggtgatttgtccatgcaaactggattaagagaagtttacttcgagttgaatggtgaaatgagaaagatcagtgtgga
agataagaaagccgcagtagaaaccgtgtcaagaccaaaagccgaccctggaaacccaaatgaagttggtgcccctatggccgg
tgtagttgtggaagtcagagttcatgagggaacagaagtgaagaaaggtgatccagtagctgtcttatctgccatgaagatggaaatg
gttatttccgccccagtctcaggtaaagtaggagaggtcccagttaaggaaggtgactctgttgatggaagtgatttgatatgcaaaatc
gtgagagcttaactcgagctagcgaagacaaccag-3′ (Y. lipolytica Pyc)
(SEQ ID NO: 207)
actagtaaatatggctgcaccaagacaacctgaagaggccgttgatgacactgagttcattgatgaccatcacgatcagca
tagagacagcgtacacaccagattgagagctaattcagcaataatgcaattccagaaaatcttagtcgccaacagaggtgagattcc
aataagaatctttagaaccgctcatgaattgtccttacaaactgtggcagtttatagtcacgaagatcatttgtctatgcatagacaaaag
gccgatgaggcttacatgattggaaagagaggtcagtatacacctgtaggagcatacttagctatagacgaaatcgtcaagattgcctt
ggaacac ggtgtgcacttaattcacccaggttatggattcttgtcagagaatgcagaatttgctagaaaagttgaacaatccggtatggt
attcgtcgga cctacccca caaactata gagagtttaggtgataaggtttctgcca gacagttggcaatca gatgtga
cgtgcctgttgtaccaggtacacctggaccagtcgaaagatacgaggaagtgaaggcttttaccgatacttatggtttccctattataatcaag
gccgcatttggtggaggtggaagaggtatgagagttgtaagagatcaagctgaattaagagactcattcgagagagccacatccgaagca
agaagtgcttttggtaacggaaccgtgttcgttgaaagattcttggatagaccaaaacatattgaggtgcagttattgggtgacaatcacggta
acgtggtacacttatttgaaagagattgtagtgtgcaaaggagacatcaaaaggtggttgaaatagcccctgcaaaagatttgccagc
tgacgtaagagatagaatcttagctgacgccgtcaagttggcaaaatcagttaattacagaaacgctggaactgccgagttcttagtgg
atcagcaaaatagatattacttcattgaaattaacccaagaatacaagttgaacacaccatcactgaggaaattaccggtatagatatc
gtagcagctcagattcaaatagccgcaggagctacattggagcagttaggtttgactcaagacagaatttccaccagaggtttcgcaa
tccaatgtagaattacaactgaagatcctagtaagggattttctccagacacaggaaaaatagaagtctatagatcagctggtggaaa
tggtgttagattagatggaggtaatggtttcgccggagcaatcattacccctcattacgattctatgttggtgaaatgcacttgtagaggttc
cacatatgagatcgccagaagaaaggtagtcagagccttagttgagtttagaatcagaggtgtgaaaactaacattccattcttgacct
ccttattgtcacaccctgtgtttgtggatggaacatgctggactaccttcatagatgacacaccagaattatttgcattggtcggttctcaga
atagggctcaaaagttattggcctacttaggagatgttgcagtgaacggttccagtattaaaggtcaaatcggagagcctaagttgaaa
ggtgacattataaagccagtattacatgatgctgccggtaaacctttggatgtctcagttccagcaactaagggatggaaacagatctta
gactctgaaggtcctgaggcttttgctagagccgtgagagcaaataagggatgtttgattatggataccacatggagggacgctcatca
atccttattggccactagagttagaaccatagacttattgaacattgcacacgagacaagtcatgctttagccaatgcatattcattggaa
tgttggggtggtgctactttcgatgtagcaatgagattcttatacgaggacccatgggatagattgagaaaattaagaaaagcagtccct
aatatcccattccaaatgttgttaagaggagctaatggtgttgcctattcttccttgccagacaacgcaatataccacttttgcaagcaggc
taagaagtgtggtgtggatattttcagagtatttgatgccttaaacgacgtcgatcaattggaagttggaatcaaagcagtgcatgctgcc
gaaggtgtagttgaggcaacaatttgctattcaggagatatgttaaacccttctaagaaatacaacttgccatactacttagatttggtcga
taaggttgtgcagttcaaacctcacgtattaggtataaaggatatggctggtgtcttgaaaccacaagccgcaagattattgatcggaag
tattagagaaagataccctgacttgcctatacatgttcatacacacgactccgctggtactggtgtagcttcaatgattgcatgtgctcaag
ccggagcagatgctgttgatgccgcaaccgactctttgagtggtatgacatctcagcctagtatcggagctatcttagcctcattggaag
gtactgagcatgatccaggtttaaacagtgcacaagtgagagctttggacacatattgggcccaattaagattgttatactctccttttgaa
gcaggattgactggtccagatcctgaagtctatgagcacgaaataccaggtggacagttaaccaacttgatcttccaggcttcacagtt
aggtttgggacaacaatgggccgaaacaaagaaagcatacgagtctgctaatgacttattgggtgacgttgtgaaagtaactcctacc
tccaaggtcgttggtgacttagcccagtttatggtaagtaacaaattgacagcagaggacgttattgctagagccggagagttagattttc
caggttcagtgttggagttcttagaaggtttgatgggacaaccatatggtggatttcctgagccattaagaagtagagcattgagagaca
gaagaaagttagataaaagacctggtttgtacttagaaccattggacttagctaagatcaaatcccaaattagagaaaattatggtgct
gccactgagtacgacgtcgcaagttatgctatgtaccctaaggttttcgaagattataagaagtttgtggccaaattcggagacttgtcag
tattaccaaccagatacttcttggcaaagcctgaaatcggtgaggagttccatgtcgaattagagaaaggtaaggttttgatattaaagtt
gttagctattggaccattgtctgaacagacaggtcaaagagaggtgttttatgaagttaacggagaagtgagacaggtgtccgttgatg
ataagaaggccagtgtggagaatactgcaagacctaaagctgaattaggtgactcatctcaggtgggagccccaatgtccggagtc
gttgtagaaatcagagttcatgatggtttggaggtgaagaaaggtgaccctattgcagtcttatcagctatgaagatggaaatggttatat
ctgcacctcacagtggaaaagtgtcctcattgttagtaaaggaaggtgattctgtcgatggacaagacttggtttgcaaaatcgtgaagg
cttaactcgagctagcgaagacaaccag-3′ (A. niger Pyc)
(SEQ ID NO: 208)
actagtaaatatgttttccaaagttttggtagctaatagaggtgagattgccataagagccttcagagctgcatatgaattaggagcc
ag aactgtcgctgtctttccatacgaagatagatggtcagagcatagattgaaagccgacgaggcttacgagatcggagaaagaggac
accctgttagagcttacttggacccagaagcaattgtagcagtcgccataagagccggtgccgatgcagtgtatcctggttacggtttctt
gtccgaaaacccagcattggccgaggcctgtgcaaacgctggtatcacatttgtaggtcctaccgccgatgtattgactttaacaggta
acaaagcaagagcaattgccgcagctaccgctgccggtgtccctactttagcaagtgttgaaccttctactgacgtggacgccttggtg
gaatcagccggagagttgccatacccattattcgtaaaggcagtggctggtggaggtggtagaggaatgagaagagttgatgcacc
aggtcaattgagagaagcagttgagacatgtatgagagaagctgaaggtgcatttggcgaccctactgtattcatagagcaggctgtc
gttgatccaagacatatcgaagtgcaagtattggcagacggtgaaggtcacgtaatgcatttgtttgagagagattgttccgtccagag
gagacaccagaaagtgattgaaatcgcccctgctccaaacttagacccagagttgagagacagaatatgcgcagacgccgttagat
tcgctaaggaaatcggatacagaaatgccggtactgtcgagttcttattggacgcaaaaggaacctatcatttcattgaaatgaatccta
gaatacaagtcgagcatacagtgactgaagaggtgacagatgtagacttagtacagagtcaattgagaatcgcttctggtgaaacctt
agccgacttgggattatcacaagaaactgtaaccttgagaggagctgcattgcagtgtagaattactacagaggacccagctaacaa
ctttagacctgacactggtgttatcacaacttacagatccccaggaggtggaggagtgagattggatggtggtactgtgtatactggtgc
cgaagtcagtgcccactttgattctatgttagctaagttgacttgcagaggtagaaccttcgagaaagccgttgagaaggcaagaaga
gctgtggccgagtttagaatcagaggtgtttcaacaaacattcctttcttgcaagccgtattggtggacccagacttttccagtggacatgtt
actacctctttcattgaaacacacccacaattattgcaagccagatcatctggtgacagaggaagcagattgttgcattacttagccgat
gtgactgtgaatcaaccacacggtcctgcacctgtttccatcgaccctgttaccaaattgccagaggtgaacttagacgttcctgctcca
gatggtacaagacagttgttgttagatgttggaccagaagagtttgccagaagattaagagcacaaactggtgttgctgtaaccgatac
aactttcagggacgcccatcaatcattgttagctaccagagtgagaacaagagatttgttagctgtagccggtcatgtcgcaagaacta
cccctcagttgtggtctttagaggcttggggaggtgccacatatgatgtagccttaagattcttagctgaggacccatgggagagattgg
cagccttaagacaagcagtgcctaacatctgtttgcagatgttattgagaggaagaaatactgtaggttacacaccttatccagccgat
gttactcaagcattcgtcgaagaagctgccgcaaccggtattgacgtgtttagaatatttgatgctttaaacgatgtggagcaaatgagg
ccagccatagaggctgtaagagctacaggaactgccgtcgcagaagttgcattgtgttacacaggagacttatccgatcctgacgag
acattgtatactttagattactatttggaattagccgatagaattgtagacgccggagcacacgtcttagctataaaggatatggcaggat
tattgagagtgccagctgccagaaccttagtcacagcattgagagacagattcgacttgccagttcatttgcacactcatgatacccca
ggtggacagttagctacattattggcagccattgacgccggtgtggatgctgtagacgccgcaactgctagtatggcaggaacaacat
cacaacctccattgtctgcattagtttccgctactgatcatggacctagagaaaccggtttgagtttaggtgccgtgtcagcattggagcc
atattgggaagctacaagaagagtatacgcacctttcgagtctggattaccttccccaactggtagagtttatagacacgaaatccctgg
aggtcaattgtcaaacttaagacagcaagctatcgccttaggtttgggagagaaattcgagcaaatagaagatatgtacgcagctgcc
aacgacatattaggtaatgtggtcaaggttaccccatctagtaaggtagtaggtgacttagcattgcacttagtcgctgttggagccgac
cctacagaatttgcagatgagccaggaaaattcgatattcctgactccgtaataggattcttaaatggagaattgggtgacccacctgg
aggttggccagaacctttcagaactaaggccttagctggtagaactcacaagcctcctgttgaggaattagacgatgaacagagaga
gggattggccggttcatctccaacaagaagaagaactttaaacgaattgttatttccaggtccaacaaaggagttcacagaaagtaga
ttaagatatggtgacacttctgtgttaccaacattggattacttatatggtttgagaagaggagaagagcatgcagtcgaaatcgaagag
ggtaaaacattaatcttgggagttcaagccataactgaacctgatgaaagaggattcagaaccgtgatgacaactattaacggtcagtt
aagaccagtgagtgtcagagacagatcagttgccgctgaggttgctgccgcagaaaaggcagataccagtaaacctggacacgtt
gcagccccatttcaaggtgtggtgtctatcgttgtggaggaaggtcaacaggtagccgctggagacacagtagcaactatcgaagcc
atgaagatggaggcctcaataaccgcacctgttgccggaacagttgagagattggccttatctggtactcaagcagtagaaggaggt
gatttggtcttagttttgtcctaactcgagctagcgaagacaaccag-3′(Nocardioides sp. Pyc)
(SEQ ID NO: 209)
ctggtgagacctctagaaaacatgaatgttttggtatacaacggtccaggttctacacctgaatcagtcaaacatgctacag
agtccttaagaaagttgttaagtccatactattctgtgcacaatgttgatgcagaagtaattaagaacgagccttggaccgaatcaactg
ccttgttagtcatgccaggtggagctgacttgccttactgttccgacttaggtggtccaggaaataagttgataagaaactggatcagag
ccggtggaaaatacttaggattttgcgccggtggatactatggtgctcaaagagtggagttcgaggaaggtacagacttggaagttatt
ggagatagagagttaagtttgtacggtggaaagtgtgtaggttctgcatataaaggttttgtctacgactcacatgccggagctagagca
gttggtgtgaattggaagggttcccctttcaaatgctactttaacggaggtggagttttcgtaccaggtaaggatatggataccgaaaata
ctgaagtcgtggctgagtacagtcaggacacagaagttcctaactctggtagatcagccgtagtcaaaatgaatgttggagagggtag
agcagtgttatccggtatacacccagagtttaacccatccatgatgaagaaaggagatcaacatatcgacgctgttattgaagagttgg
aaaacttcgaaaaggagagattagccttcttgagacacttaatgaccttgttaggtttgaaaactaatccagatacaaccgatatgacttt
aacatctttgtatgtaactggaaacggtgtcgcaaaattattgaaggacttagatgtgtcagaagaaaatagagttttctctgctcctaac
gacaccttcttctttggtgagaaaccatccggagatagtaatcatacacatgtaataccaatggtcggtgatgttcctgcctcagaattga
ctccacactttgaccataagttatactatcagtctttgagagcacctgaattaggatcaactttgttatacggagaggtgttgacaagtactt
caacattattggataagaactacaacttattgagacacttaccaaacggtttcaccgctgttggaacggtacaattgtctggtagaggta
gaggaaataatgtctgggttaaccctatcggtgtgttagccgtatccacagtcttgagaattaactttaacccattcggtcaaaatacgag
tattatatttgttcagtacttagcatcattggctatggtgcaagcaatcaagaactatggacctggttattctgaagttccagtgaaattaaa
gtggcctaatgacatctacgcagctaacccaggctccgagatggtcggtagtaccgatgcttatttgaaaataggtggagttatcgtga
actctaacgtattcgatggtcaatacatgttagtcgttggttgtggagtgaatgttacaaatagtgcccctactacctccttgaacatgttaat
taattcaatgaacgaaaagaatggtacaactttggaacattatagaaccgaggtattattggcaaaattcttagaaacattcgaagctat
gatggacgcctttaagaaccacggattctctatatttgagccattgtactatagttcttggttacatcaggatgcacaagtcagattggaac
attacggtaatgttaaagctactgtgaaaggtatctccatggaccagggaatgttattggtacaagaagagggttcaggtagagtcattg
aattacaacctgatggaaacagttttgatatgatgagaggtttgttaaagagaaaagagtaagtcgacggtctcagtcg-3′ (Y.
lipolytica Bpi)
(SEQ ID NO: 210)
ctggtgagacctctagaaaacatggctactccaaatatgacaggtaagaaggttaacgtattggtctattctggagcaccttt
atcaccattgctgcctgcccaacatcagagatacccatccttgtgtacccaaagaattataagaaatggtactacagtggaaagtgtta
gacacaccttatattctttgagaagattattggctcctcattacgcagtaatcccagtcacttcagatgccttattgcacgagccttggaca
gctacgtgcgcattattggtgattccaggaggtgccgacttaggttatggaagagttttgaacggaccaggtaatagaagaatagaac
agtttgtgaaaagaggtggtgcttacttaggattctgtgccggtggttattacggctcccaaagatgcgagtttgaagtcggtgataagac
tttgcaagttatcggagaaagagagttagctttctatccaggtacatgtagaggtggagcctttgccggtttcgtgtaccatagtgaagctg
gtgccagagcagctgaaatttctgttaacaaagacatattgaatgccggaatcgtacctgagagattcagatgctattacaacggtgga
ggtgtgttcgtggatgcaccaaccttagctgacaagggtgttgaagtattggcctcatttgaagaggaattaaacgtggacccaggtga
gggtaaagcagctgttgtgttttgcagagttggtgaaggaagagtagtcttgactggtccacacccagaatttgccgcagctaacttaga
taagaaagctggaggtcctgagtatacaaaggtgattgaagccttggaagcagacgataaagctagaactgacttcttaaaggcctg
tttggtgaaattaggtttgcaagtaacccaatccacaactaccgtcccaagtttatcttcattgcacttatccagtcaagagcccgcagaa
acagctgatttggttgcctcttggcaggaaatcatcaccaaagatggaaatgaggaaatcattaaagacgaaaatgatacctttagaa
tagagaggccaggtgcatggaacttatcacaattggaggactctttacctgagtccagtcagtcaaccgaaggtatcgtggattacaat
gctattgttaaaagattggtagtccatgatgacgttccatcttccaagttaactccttactttaatcaccatgcattctacagtaatttgcacca
atatcaatcacagagcagagaaggagcttccgagtttggtgcccacttagtgtacggagaagtagtcacaagtactaacaccatattg
gaaaagaatccaaagttattgagaaaattacctaacggtttcacagcatcagctactacccaagttgccggtagaggaagaggttcta
acgtgtgggtttccccagccggtgctttgatcttttcaacagtattaagacaccctttggagaagattcaatctgccccagtcgttttcattca
gtacttagcagctatggccgtggtacaaggaatcaagaactatgatgccggttacagtgaattgcctgtcaagttaaagtggccaaatg
acgtttatgctttggacccagaacatcctgagaagaaacagtactccaagatctgcggaatcttagtgaactcacattattgtgctaacg
aatacatatctgttgtaggtatcggtattaatgccactaacgcaagtccaaccacatctttgactgctttagccgcaagattcttgggacct
agagctgccccaataaccttagagaaattgttagcaagaattttgacaactttcgaagaattatacaccagattcttgagatccggtttcg
atagatcatttgaggaaatgtactatgctgactggttacacatgcatcaagtcgttacattggaagaggaaggtggagtgagagccag
aatcaaaggtattactagagattacggattattgttagcagaagagttgggttggaatgacagacctaccggtagagtatggcaattac
agagtgattctaactcatttgatttctttagaggattggtcagaagaaaggtttaagtcgacggtctcagtcg-3′ (A. niger
Bpi)_

Plasmids harboring the Pyc-encoding genes are constructed by treating the synthetic DNA with SpeI and XhoI and ligating to XbaI-XhoI-cleaved pRS426TEF, pRS426ADH1, or pRS426TDH3. These are introduced into strains in which overexpressed Mdh-encoding constructs have been integrated at the can1 locus, and which also express OAT_{Mal}.

Plasmids harboring the Bpl-encoding genes are constructed by treating the synthetic DNA with XbaI and SalI and ligating to XbaI-XhoI-cleaved pRS413TEF, pRS413ADH1, or pRS413TDH3. These are introduced into his3 strains as described in Example 21 herein for S. cerevisiae BPL1.

In order to co-express these Pyc- and Bpi-encoding genes with one another in various combinations, the PYC2 promoter present in pRS2MDH3L1SKL is replaced with the TP/1 promoter by amplifying a 450 bp fragment from S. cerevisiae DNA with M05314 (5′-CACACCTGCAGCCGCGGGATTTAAACTGTGAGGAC (SEQ ID NO: 211)) and M05315 (5′-CTCTCACTAGTTTATGTATGTGTTTTTTGTAGTTATAG (SEQ ID NO: 212)), and cleaving it with Pstl and Spel; amplifying pRS2MDH3L1SKL DNA with M05316 (5′-CACACACTAGTAAAATATGAGCAGTAGCAAGAAATTG (SEQ ID NO: 213)) and M05317 (5′-GACGTTCCCATGGATCCTCAT (SEQ ID NO: 214)), and cleaving the resultant 1.8 kb fragment with BamHI and Spel; and ligating both fragments to Pstl- and BamHI-cleaved pRS2MDH3U1SKL to yield pMB4972.

In addition, a sequence corresponding to a PYC2/CYC1 hybrid terminator for convergently transcribed genes is synthesized:

(SEQ ID NO: 215)
5′-ggtctcaccagtttttactcgttaattatattttatgacatctgaaa
atactagctgtactatatatggcgtatattttatctagttatgttcccat
gtatatttaaatgccaaatagaaagtaatcaaacactttcgatgaaatac
gtgctaactgtgtttcttccttaatgctttcacttaccatgtctccattc
tccattttcttcttgagtgaaaatgtgagtttataacgctcaagtacgtt
aactactctatttaatatcgtacgggatttttgatcgactgtaggttttc
ttcttagaccattccagcggccgcaaattaaagccttcgagcgtcccaaa
accttctcaagcaaggttttcagtataatgttacatgcgtacacgcgtct
gtacagaaaaaaaagaaaaatttgaaatataaataacgttcttaatacta
acataactataaaaaaataaatagggacctagacttcaggttgtctaact
ccttccttttcggttagagcggatgtggggggagggcgtgaatgtaagcg
tgacataactaattacatgactcgactgagacc-3′ (PYC2/CYC1
terminator)

Any combination of synthetic Pyc/Bpl-encoding genes may be made by linearizing any pUC19-based plasmid harboring a Pyc-encoding gene with BbsI, and inserting compatible BsaI fragments from the terminator sequence and from either Bpl-encoding gene. The PYC-term-BPL cassette may then be moved to XbaI- and SpeI-treated pMB4972 as a XbaI-SpeI fragment in either orientation. The resultant plasmids can be introduced into strains expressing OAT_{Mal} and Mdh as described in other examples herein.

### Example 25

### Expression of Heterologous Phosphoenolpyruvate Carboxylase (Ppc) Polypeptides

The gene encoding Ppc is amplified from Erwinia chrysanthemi and is used in a manner similar to that described for PEPck in example 19 herein, to supplement or replace the PYC2 gene resident in pRS2MDH3L′1SKL. M03764 (5′-ATGAATGAACAATATTCCGCCA-3′ (SEQ ID NO: 216)) and M03765 (5′-TTAGCCGGTATTGCGCATCC-3 (SEQ ID NO: 217)) were used to amplify a 2.6 kb fragment that was subsequently ligated to Smal-cleaved pBiuescriptiiSK- to create pMB4077. This plasmid may be cleaved with Pstl and BamHI, the ends made blunt with the Klenow enzyme, and ligated to the URA3 vectors (likewise made blunt, for example by cutting with Xbal and Xhol and made blunt with Klenow) outlined for PEPck in example 19 herein. The resultant expression cassettes may be moved as Saci-Xhol blunted fragments to pRS2MDH3L′1SKL as described for PEPck in example 19 herein.

The resultant plasmids are used in place of pRS2MDH3ΔSKL in the Pdc⁻ strains described above containing YEplac112SpMAE1, and assayed for malic production. Alternatively, they may be used in pyk1 or pyk1 pyk2 strains deficient in pyruvate kinase activity in conjunction with YEplac112SpMAE1.

### Example 26

### Additional Strategies to Increase Malic Acid Production

Several enzymatic and transport activities present in yeast cells have the potential to reduce malic production: the permease encoded by JEN1, which possibly mediates the unwanted export of pyruvate; phosphoenolpyruvate carboxykinase (PCK1 gene product) and the malic enzyme (MAE1 gene product), which can reverse Pyc-mediated carboxylation; fumarase (FUM1 gene product), which can catalyze the dehydration of malate to produce fumarate; and RTG3, which positively regulates TCA enzyme genes that operate in the oxidative direction and as an aggregate deplete oxaloacetate pools.

These genes may be deleted, using PCR amplification of DNA from the appropriate strain in the yeast deletion set (American Type Culture Collection (ATCC) Catalog # GSA-4 (MATa haploid); GSA-5 (MATalpha haploid); GSA-6 (heterozygous diploid)), each of which contain a G418^{R} cassette replacing the coding region of the gene in question. Primers are chosen from a region approximately 500 bp upstream and 500 bp downstream of the gene in question, a 2.5 kb geneΔ::G418^{R} fragment is amplified and used to transform any of the above strains to resistance to 100 mg/L G418. These strains, when harboring the appropriate Pyc/Mdh and OAT_{Mal} expression plasmids, may be tested for malate production in shake flask fermentations.

In another approach, a mutation in FUM1 can be engineered that prevents the appearance of fumarase in the cytosol, but permits its expression in mitochondria.

In order to increase biotinylation of Pyc, the biotinylation site of Arc1, a known biotinylated protein and potential substrate competitor for or regulator of biotin protein ligase enzymes, is mutated. Primers M05663 (5′-CACACCGTCTCTCTAGACGCCTCTAGCTTGACGC-3′ (SEQ ID NO: 218)) and M05664 (5′-CACACCGTCTCACTCTGGAGCTTGCCACTAAATCCTTAATC-3′ (SEQ ID NO: 219)); and M05665 (5′-CACACCGTCTCAAGAGATGTCAAGTCAACTTATACCACAT-3′ (SEQ ID NO: 220)) and M05666 (5′-CACACCGTCTCGGTACCATTTGCAATTGGGTAGG-3′ (SEQ ID NO: 221)); are used to amplify portions of the ARC1 gene (0.97 and 1.2 kb, respectively), that upon digestion with BsmBI and ligation together into Acc651- and Xba-cleaved pRS406, yields a construct containing the ARC1-K86R allele, abolishing biotinylation of the encoded Arc1 protein. Digestion of the resulting plasmid with BsrGI or Bg/11 targets integration to the ARC1locus, and excisants selected on 5-fluoroorotic acid include ura3-strains harboring an ARC1-K69R allele. The manipulation is performed in a variety of malate production hosts, and such strains, when harboring the appropriate Pyc/Mdh and OATMal expression plasmids, are tested for malate production in shake flask fermentations.

Succinate and Fumarate Production:

In order to convert malate to fumarate, a plasmid overexpressing the cytosolic form of Fuml is constructed. The primers FumF (CACACTCTAGAAACAAAATGAACTCCTCGTTCAGAACTG) and FumR (CACACCTCGAGCTCGTTTATTTAGGACCTAGC) amplify a 1.4 kb FUM1Δss fragment that is missing the sequences (nucleotides 1-69) that encode a mitochondrial targeting signal. The fragment is cleaved with XbaI and XhoI and ligated to XbaI-XhoI-cleaved pRS413TEF or pRS413TDH3. The resultant plasmids are introduced into the his3 hosts described for BPL1 and NCE103 in other examples herein, which also contain Pyc- and Mdh-expressing plasmids, as well as the appropriate OAT_{Fum} expression cassette.

In order to convert malate to succinate, a strain containing or lacking the FUM1Llss expression construct is further modified by the introduction of a plasmid overexpressing one of the two genes (FRDS1) that encode fumarate reductase. The primers FrdsF (5′-CACACTCTAGAAACAAAA TGTCTCTCTCTCCCGTTGTTG-3′ (SEQ ID NO: 224)) and FrdsR (5′-CACACCTCGAGCGTTACTTGCGGTCA TTGGCAATAG-3′ (SEQ ID NO: 225)) amplify a 1.4 kb FRDS1 fragment that is cleaved with Xbal and Xhol and ligated to Xbai-Xhol-cleaved pRS413TEF or pRS413TDH3. The resultant plasmids are introduced into the his3 hosts described for BPL 1 and NCE103 in other examples herein, which also contain Pyc- and Mdh-expressing plasmids, as well as the appropriate OATsue expression cassette.

If the expression of both FUM1Δss and FRDS1 is desirable, any of the expression cassettes described above may be excised with SacI and XhoI, made blunt with T4 DNA polymerase, and ligated to pRS2MDH3ΔSKL treated with MluI and the Klenow enzyme. This permits the construction of a strain bearing three plasmids: Pyc-Mdh-Frds (URA3), Fum (HIS3), and OAT_{Suc} (TRP1); or Pyc-Mdh-Fum (URA3), Frds (HIS3), and OAT_{Suc} (TRP1).

### Example 27

### Expression of an Acid Transporter to Increase C4 Acid Production

Production of organic acids, e.g., malic acid can be increased in a fungal cells by modifying the fungal cell to express a protein (e.g., a dicarboxylic acid transporter or exporter/importer) that allows export of an organic acid such a as C4 organic acid. This permits export of organic acids that might otherwise suppress additional organic acid synthesis.

A sequence encoding a putative dicarboxylic acid transporter from Aspergillus oryzae (GenBank Accession No. XP_{—}001820881; DCAT) was synthesized. The sequence used, optimized for S. cerevisiae codon bias, follows.

(SEQ ID NO: 226)
TTCTAGAAACAAAATGTTT AATAACGAGCA TCATA
TACCTCCTGGATCTAGCCACTCGGATATTGAAATGTTAACTCCTCCTAAA
TTTGAAGATGAAAAACAACTTGGACCTGTCGGTATAAGAGAAAGACTTAG
ACACTTTACTTGGGCTTGGTATACACTAACTATGAGTGGGGGCGGCTTAG
CTGTTTTAATAATTTCACAACCTTTTGGTTTCAGAGGTCTTAGGGAAATC
GGAATCGCTGTTTATATTCTAAATCTTATACTTTTTGCTTTAGTTTGTTC
CACTATGGCTATTAGGTTTATACTACATGGTAATTTATTAGAAAGTTTGC
GTCATGATAGAGAAGGTTTGTTCTTTCCCACATT
CTGGCTTTCAGTTGCAACAATTATATGTGGTTTATCAAGGTATTTCGGTG
AAGAATCAAATGAAAGTTTTCAGCTAGCTTTAGAAGCTCTGTTCTGGATT
TATTGCGTTTGTACACTATTAGTAGCTATTATACAATATTCATTCGTTTT
CTCCTCTCATAAATATGGTCTACAAACTATGATGCCATCTTGGATACTAC
CAGCTTTTCCTATAATGTTGTCAGGTACTATTGCGTCTGTTATTGGCGAG
CAACAACCAGCTAGAGCAGCTTTACCTATAATCGGAGCAGGTGTAACTTT
TCAAGGATTAGGTTTTTCAATTTCTTTTATGATGTATGCACACTATATTG
GTCGTCTAATGGAATCTGGTTTACCACACTCAGATCATAGACCTGGTATG
TTTATATGTGTTGGTCCACCGGCCTTTACAGCACTAGCCTTAGTCGGTAT
GTCTAAGGGTTTGCCTGAAGATTTTAAGTTATTACATGATGCACACGCCC
TGGAAGATGGAAGAATTATAGAACTATTAGCAATCTCTGCAGGTGTTTTC
TTATGGGCTT
TAAGTTTATGGTTTTTTTGTATTGCAATTGTCGCCGTTATCAGATCACCT
CCCAAAGCCTTTCATTTAAACTGGTGGGCTATGGTTTTCCCAAACACTGG
TTTCACTTTAGCAACAATAACCCTAGGTAAAGCATTAAACTCTAACGGTG
TAAAAGGTGTTGGTTCAGCTATGAGTATTTGTATTGTATGTATGTATATA
TTCGTTTTCGTAAATAATGTTAGAGCTGTGATACGTAAAGATATAATGTA
CCCTGGTAAAGACGAAGATGTCTCTGATTAGTCTTCTCGAG

The amino acid sequence of the encoded transporter follows (GenBank ______).

(SEQ ID NO: 227)
MFNNEHHIPPGSSHSDIEML TPPKFEDEKQLGPVGIRERLRHFTWAWYT
LTMSGGGLAVLIISQPFGFRGLREIGIAVYILNLILFALVCSTMAIRFIL
HGNLLESLRHDREGLFFPTFWLSVATIICGLSRYFGEESNESFQLALEAL
FWIYCVCTLLVAIIQYSFVFSSHKYGLQTMMPSWILPAFPIMLSGTIASV
IGEQQPARAALPIIGAGVTFQGLGFSISFMMYAHYIGRLMESGLPHSDHR
PGMFICVGPPAFTALALVGMSKGLPEDFKLLHDAHALEDGRIIELLAISA
GVFLWALSLWFFCIAIVAVIRSPPKAFHLNWWAMVFPNTGFTLATITLGK
ALNSNGVKGVGSAMSICIVCMYIFVFVNNVRAVIRKDIMYPGKDEDVSD

The transporter-encoding nucleic fragment was liberated from its vector using XbaI and XhoI, and ligated to XbaI-XhoI-cleaved pRS416GPD to create pMB5210 (CEN URA3). The TDH3p-DCAT1-CYC1t cassette was moved to pRS404 using KpnI and SacI to create pMB5238 (integrating TRP1). Spontaneous Trp⁻ revertants were obtained from MY2888 and MY2907 as fluoro-anthranilate-resistant clones, and MY3229 (Pyc⁻) and MY3230 (Pyc⁺) were identified as having simultaneously lost TRP1 and TDH3-Spmae1 by homologous excision. Next, pMB5238 was used to transform MY3230 to prototrophy (via integration at the trp1 locus), creating MY3523, MY3524, and MY3525. Alternatively, pMB5238 was used to transform MY3229 to tryptophan prototrophy (via integration at one of two resident CYC1 terminators), creating MY3300, which was subsequently transformed to uracil prototrophy with pMB5165 (directed to integrate at the pyc2 locus), creating MY3522. These four Pyc⁺ Dcat⁺ strains are predicted to be virtually genetically identical, and they behave similarly in fermentations. On average the four strains were capable of producing greater than 16 g/L malic acid in 96 hr when cultured with 100 g/L glucose and 0.5% CaCO₃. In comparison, strain MY2907, containing the S. pombe mae1 transporter instead of DCAT1, typically produces 12 to 15 g/L malic acid under these poorly buffered conditions (final pH<3).

Additional useful organic acid transporters are listed in FIGS. 35 and 62.

### Example 28

### Production of Malic Acid in Low pH Cultures

Fungal strains used for production of malic acid are generally culture at around pH 4.5. However, maintaining this pH during malic acid production can require the use of significant quantities of CaSO₄, which can be costly both in terms of cost of materials and disposal of waste products. Thus, the ability to produce malic acid in lower pH cultures (e.g., pH 2.5) would have significant benefits with respect to the economics and environmental impact of the downstream processing and purification steps. For instance, a hundred-fold reduction in the amount of CaSO₄ would be possible if the final pH could be reduced from pH 4.5 to pH 2.5.

Reduced buffering and/or low pH culturing is difficult during the production of malic acid because the production of pyruvate by pdc1 pdc5 strains leads, at low pH, to the generation of protonated pyruvic acid, which is toxic. To address this issue, a pyc1 pyc2 strain, MY2888, whose malic production could be increased upon introduction of a Pyc-encoding plasmid, whose flux to ethanol is reduced but not eliminated, and which secretes undetectable levels of pyruvate. Described below are strains derived from MY2888 which produce high levels of malic acid even when cultured at low pH.

TAM was cured of an episomal URA3 plasmid, rendered trp1ΔhisG using a hisGURA3-hisG cassette, and a TDH3p-MDH3ΔSKL cassette was integrated at the can1 locus by URA3-mediated integration and excision to create MY2421. Subsequent integration of a TDH3p-Spmae1 TRP1 plasmid (pMB4957) at the same locus yielded MY2542.

A pyc1 pyc2 strain (CMJ238) of the W303 background was obtained from Carlos Gancedo (University of Madrid). After HO-mediated mating type switching to MATa to create MY2682, this strain was mated to MY2542, sporulated, and a glucose-ammonia-negative antimycin-sensitive spore was identified, MY2888. Its genotype was determined to be pyc1 pyc2 PDC1 pdc5 PDC6 can1::TDH3p-MDH3ΔSKL::TRP1::TDH3p-Spmae1 MTH1ΔT. Introduction of TDH3p-PYC2 in a single copy at the pyc2 locus (pMB5165) of MY2888 results in a strain, MY2907, capable of substantial malic production (see Table below). Introduction of multiple episomal copies of TDH3p-YlPYC (pMB5094) into MY2888 results in a strain, MY2928, with even higher productivity (Table XX). Moreover, the lack of pyruvate secretion allows for the malic production under poorly buffered conditions (Table XX, Column 4).

Typical malic acid productivity in shake flasks (g/L)

| | 100 g/L Glu 50 g/L | 200 g/L Glu 100 g/L | 100 g/L Glu Ca- |
|---|---|---|---|
| Strain | CaCO₃ | CaCO₃ | MES 20% CO₂ |
| MY2907 | >55 | >100 | ~20 |
| MY2928 | >65 | ~140 | ~30 |
| final pH | ~5 | ~5 | ~2.5 |

Media conditions with CaCO₃ were according to Verduyn, lacking ammonium sulfate and containing 1 g/L urea. In the third column, the same medium was used with 13 mM Ca-MES (2 mM Ca⁺⁺) pH 5.7 in a 20% CO₂ atmosphere.

Plasmid pMB5165 (TDH3p-PYC2 URA3) was prepared as follows. Oligo M05316 (CACACACTAGTAAAATATGAGCAGTAGCAAGAAATTG (SEQ ID NO: 228)) was used to insert a Spel site upstream of the PYC2 open reading frame in pRS2MDH3b.SKL by PCR amplification (pMB4972; also contains the TP/1 promoter in place of native PYC2 promoter). A fragment comprising the PYC2 open reading frame and the PYC2 terminator was subsequently ligated as a 3.5 kb Spei-Bs1WI fragment to Spei-Acc651-cleaved pRS414GPD to create pMB5099. The TDH3p-PYC2 cassette was then moved as a Bg/1 fragment to Bg/1-cleaved pRS406 to create pMB5165.

Plasmid pMB5094 (TDH3p-YIPYC URA3 2m) was prepared as follows. A nucleic acid molecule having the sequence below, encoding the Y. lipolytica pyruvate carboxylase using S. cerevisiae codon bias, was synthesized:

(SEQ ID NO: 229)
actagtaaatatgtctaatgttccagaaactaaagtagatccttcattgtccacaccagaggtccctagtcaaggtttacatagcagattg
gacaagatgagagctgattcatccatattgggaagtatgaacaaaatattagtggcaaatagaggtgaaatcccaattagaatctttag
aaccgcccacgagttatctatgcagactgttgctatctatgcacatgaggacagattgtcaatgcacagattcaaggccgatgaggctt
acgtaattggagacagaggaaaatatacacctgtccaagcatacttacaggtggacgagataatcgaaattgccaaggctcatggtg
ttaacatggtacacccaggatatggtttcttgtccgaaaatagtgagttcgcaagaaaagtcgaagaagctggaatggcctggattggt
cctccacataacgttatagaca gtgtcggtga caaggtttcagcaa gaaaettagctatcaa gaacaatgta cctgtcgtgcca ggaa
ccgatggtcctgttgaggacccaaaggatgccttgaaatttgtagaaaagtacggttatcctgtcattataaaagcagctttcggaggtg
gaggtagaggtatgagagttgtgagagagggagatgacatcgttgatgcctttaacagagcatccagtgaagctaagactgccttcg
gtaatggtacatgtttcattgaaagattcttagacaaaccaaaacatatagaggtacaattgttagcagatggacaaggtaatgtcgtgc
acttgtttgaaagagattgctctgttcagaggagacatcaaaaggtagtcgaaatcgctccagccaaagacttacctgtcgaggtgag
agatgcaattttggacgatgctgttagattagctgaagatgccaagtacagaaacgcaggaaccgctgagttcttggtagacgagcaa
aatagacactacttcattgagataaacccaagaatccaggtcgaacatactattacagaggaaataaccggtatcgatattgttgccg
cacaaatacagattgctgccggtgcaactttagagcaattgggattaacacaagacaaaatctcaactagaggttttgctattcagtgta
gaataaccacagaagatcctgcaaagcaattccaaccagatactggaaaaatcgaagtctacagatctgctggaggtaatggagta
agattggacggtggtaacggatttgccggtgcaattatatcccctcactatgatagtatgttagtcaagtgctcatgttctggcaccacattc
gagatagccagaagaaagatgattagagccttggttgagtttagaataagaggagtcaagactaatattccattcttattggcattattga
cacatcctacctttatcgaaggaaaatgctggactacattcattgacgatactccatccttatttgacttgatgaccagtcagaacagggc
tcaaaagttattggcctacttagcagatttatgtgttaatggaacaagtataaaaggtcaggtaggtaaccctaagttaaagtctgaggtc
gttatcccagtgttgaagaactccgaaggaaagattgtagattgtagtaaacctgacccagtcggttggagaaatatattagttgaaca
aggtcctgaggctttcgccaaggcagtgagaaagaacgatggagttttggtaatggacactacctggagagatgctcatcaatcattat
tggctacaagagtcagaactaccgacttattggcaattgcaaatgaaacatctcacgctatgtccggtgcctttgcattagagtgctggg
gaggtgctacttttgacgttgcaatgagattcttgtatgaagatccatgggacagattaagaaagatgagaaaagcagtgccaaatatc
ccttttcagatgttgttaagaggtgctaatggagtagcctactcatctttgccagataacgcaatagatcatttcgtcaagcaagctaaag
acaatggtgttgatatctttagagtgttcgacgccttaaacgatttggatcaattaaaggtaggtgttgacgcagtcaagaaagctggag
gtgttgtggaagcaaccgtatgttatagtggagatatgttgaatcctaagaagaagtacaacttagagtattacttggactttgtcgataga
gttgtagaaatgggcacccacatcttaggtattaaagatatggcaggaactttgaagccagctgccgcaaccaaattaataggtgctat
cagagaaaagtatcctaatttgccaattcatgttcatacacacgactccgccggtactggagtggcatcaatggctgccgcagctgag
gccggtgcagatgtcgttgacgtggcttctaatagtatgtctggaatgacctcccagccttcaataagtgccttaatggcaacattggaag
gaaaattatctactggtttggacccagctttagtaagagaattggatgcctattgggcacaaatgagattattgtactcatgcttcgaggct
gacttaaagggacctgatccagaagtctttcaacatgaaattcctggtggtcagttgacaaacttattgttccaagcccagcaagttgga
ttaggtgagcaatggaaagaaactaagcaggcatatatcgctgccaatcaattgttaggagacattgtaaaagttaccccaacatcta
aggtggtcggtgatttggcacagtttatggtttccaacaaattaagttacgacgatgtgataaaacaggctggttcattggattttcctggat
ctgtattagacttctttgagggtttgatgggtcaaccatatggaggtttcccagaacctttaagaactgaagcattaagaggacagagaa
agaaattaaccgagaggcctggaaaatccttgcctccagtcgattttgcagctgttagaaaagacttagaagaaagattcggtcacat
cacagagtgtgatattgccagttactgcatgtatcctaaggtatttgaagattacagaaagatagttgacaagtatggagatttgtcaattg
tgccaactagattattcttggaagcacctaaaaccgacgaggaattttctgtcgaaatcgagcaaggtaagacattaatattggctttaa
gagctattggtgatttgtccatgcaaactggattaagagaagtttacttcgagttgaatggtgaaatgagaaagatcagtgtggaagata
agaaagccgcagtagaaaccgtgtcaagaccaaaagccgaccctggaaacccaaatgaagttggtgcccctatggccggtgtagt
tgtggaagtcagagttcatgagggaacagaagtgaagaaaggtgatccagtagctgtcttatctgccatgaagatggaaatggttattt
ccgccccagtctcaggtaaagtaggagaggtcccagttaaggaaggtgactctgttgatggaagtgatttgatatgcaaaatcgtgag
agcttaactcgag

This sequence was moved as a SpeI-XhoI fragment to SpeI-XhoI-cleaved pRS426GPD to create pMB5094.

Plasmid pMB4957 (TDH3p-Spmae1 TRP1) was prepared as follows. The KpnI-SacI fragment comprising TDH3p-Spmae1 from YEplac112SpMAE1 was ligated to KpnI-Saci-cleaved pRS404 to create pMB4957.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A recombinant fungal cell having a genetic modification that decreases pyruvate decarboxylase (PDC) activity and a modification that increases or decreases fumarate reductase activity, wherein the recombinant fungal cell, when cultured under conditions that produce a C4 dicarboxylic acid, produces more of at least one C4 dicarboxylic acid than an otherwise identical fungal cell not having the genetic modification.

2. The recombinant fungal cell of claim 1 having a genetic modification that increases malate dehydrogenase (MDH) activity.

3. The recombinant fungal cell of claim 1 having modification selected from the group consisting of a modification that:
a. increases anaplerotic activity;
b. increases or decreases organic acid transport activity;
c. increases or decreases glucose sensing and regulatory polypeptide activity;
d. increases or decreases hexose transporter (HXT) activity; and
e. increases or decreases C4 dicarboxylic acid biosynthetic activity.

4. The recombinant fungal cell of claim 3, wherein the modification to increase anaplerotic activity comprises at least one modification selected from the group consisting of a modification that:
f. increases pyruvate carboxylase (PYC) activity;
g. increases phosphoenolpyruvate carboxylase (PPC) activity;
h. increases or decreases phosphoenolpyruvate carboxykinase (PCK) activity;
i. increases or decreases pyruvate kinase (PYK) activity;
j. increases biotin protein ligase (BPL) activity;
k. increases biotin transport protein (VHT) activity;
l. increases or decreases bicarbonate transport activity;
m. increases carbonic anhydrase activity.

5. The recombinant fungal cell claim 3, wherein the modification to increase or decrease C4 dicarboxylic acid biosynthetic activity comprises at least one modification selected from the group consisting of a modification that:
n. increases malate dehydrogenase (MDH) activity;
o. increases or decreases fumarase activity;
p. increases or decreases malate synthase activity;
q. increases or decreases malic enzyme activity;
r. increases or decreases isocitrate lyase activity;
s. increases or decreases ATP-citrate lyase activity;
t. increases or decreases succinate dehydrogenase activity.

6. The recombinant fungal cell of claim 1, wherein the modification to decrease pyruvate decarboxylase (PDC) activity comprises at least one modification selected from the group consisting of a modification to decrease PDC1, PDC2, PDC5, or PDC6 activity.

7. The recombinant fungal cell claim 1, wherein the modification to increase or decrease glucose sensing and regulatory polypeptide activity comprises at least one modification selected from the group consisting of modifications to increase or decrease SNF1, MIG1, MIG2, HXK2, RGT1, SNF3, RGT2, STD1, MTH1, GRR1, YCK1, HXK1, and GLK1 polypeptide activity

8. The recombinant fungal cell of claim 1, wherein the genetic modification that increases or decreases glucose sensing and regulatory polypeptide activity does so by increasing or decreasing expression of a glucose sensing and regulatory polypeptide.

9. The recombinant fungal cell of claim 8, wherein the genetic modification that increases expression is the addition of a gene encoding a glucose sensing and regulatory polypeptide.

10. The recombinant fungal cell of claim 8, wherein the genetic modification that increases expression is a genetic modification that increases the transcription or translation of a gene encoding a glucose sensing and regulatory polypeptide.

11. The recombinant fungal cell of claim 8, wherein the genetic modification that decreases expression is the deletion of all or part of a gene encoding a glucose sensing and regulatory polypeptide or the disruption of a gene encoding a glucose sensing and regulatory polypeptide.

12. The recombinant fungal cell claim 3, wherein the modification to increase hexose transporter (HXT) activity comprises at least one modification selected from the group consisting of modifications to increase or decrease HXT1, HXT2, HXT3, HXT3, HXT4, HXT5, HXT6, or HXT7 polypeptide activity.

13. The recombinant fungal cell of any of claim 1, wherein the fungal cell comprises more than one modification selected from the group consisting of modifications to:
v. increase anaplerotic activity;
w. decrease PDC activity;
x. increase or decrease organic acid transport activity;
y. increase or decrease glucose sensing and regulatory polypeptide activity;
z. increase hexose transporter (HXT) activity; and
aa. increase or decrease C4 dicarboxylic acid biosynthetic activity.

14. The recombinant fungal cell of claim 13, wherein the more than one modifications are selected from the group consisting of modifications to:
bb. increase anaplerotic activity;
cc. decrease PDC activity;
dd. increase organic acid transport activity;
ee. increase glucose sensing and regulatory polypeptide activity; and
ee′ increase or decrease C4 dicarboxylic acid biosynthetic activity.

15. The recombinant fungal cell of claim 1, wherein the fungal cell comprises more than two modifications selected from the group consisting of modifications to:
ff. increase anaplerotic activity;
gg. decrease PDC activity;
hh. increase or decrease organic acid transport activity;
ii. increase or decrease glucose sensing and regulatory polypeptide activity;
jj. increase HXT activity; and
kk. increase or decrease C4 dicarboxylic acid biosynthetic activity.

16. A method of producing a C4-dicarboxylic acid, comprising: culturing a recombinant fungal cell of claim 1 under conditions that achieve C4-dicarboxylic acid production.

17. The method of claim 16, further comprising a step of: isolating a produced C4-dicarboxylic acid.

18. The method of claim 17, wherein the C4-dicarboxylic acid is selected from the group consisting of malic acid, fumaric acid, tartaric acid, and succinic acid.

19. The method of claim 16, wherein the step of culturing under conditions that achieve C4-dicarboxylic acid production comprises culturing at a pH within the range of 1.5 to 7.

20. The method of any claim 16, wherein the step of culturing under conditions that achieve C4-dicarboxylic acid production comprises culturing under conditions and for a time sufficient for C4-dicarboxylic acid to accumulate to a level within the range of 10 to 200 g/L.

21. A method of preparing a food or feed additive containing a C4-dicarboxylic acid, the method comprising steps of:
ll. cultivating the recombinant fungal cell of claim 1 under conditions that allow production of the C4-dicarboxylic acid;
mm. isolating the C4-dicarboxylic acid; and
nn. combining the isolated C4-dicarboxylic acid with one or more other food or feed additive components.

22. The method of claim 21, wherein the C4-dicarboxylic acid is selected from the group consisting of malic acid, fumaric acid, tartaric acid, and succinic acid.

23. A method of preparing a polymer containing a C4-dicarboxylic acid, the method comprising steps of:
oo. cultivating the recombinant fungal cell of claim 1 under conditions that allow production of the C4-dicarboxylic acid;
pp. isolating the C4-dicarboxylic acid; and
qq. combining the isolated C4-dicarboxylic acid with one or more polymer components.

24. A method of preparing a C4-dicarboxylic acid derivative, the method comprising steps of:
a) cultivating the recombinant fungal cell of claim 1 under conditions that allow production of a C4-dicarboxylic acid;
b) isolating the C4-dicarboxylic acid; and
c) converting the isolated C4-dicarboxylic acid into a C4-dicarboxylic acid derivative.

25. The method of claim 24 wherein the C4-dicarboxylic acid is chosen from one or more of malic acid, fumaric acid, tartaric acid, and succinic acid.
